(19) United States
(12) Patent Application Publication
(10) Pub. No.: US 2004/0068096 A1
(43) Pub. Date: Apr. 8, 2004

(54) Human single nucleotide polymorphisms in organic anion transport and multi-drug resistant proteins

(76) Inventors: Zenta Tsuchihashi, Pennington, NJ (US); Lester Hui, Fairfax, VA (US); Todd Kirchgessner, North Wales, PA (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/252,155

(22) Filed: Sep. 20, 2002

Related U.S. Application Data
(60) Provisional application No. 60/324,172, filed on Sep. 21, 2001.
(60) Provisional application No. 60/333,700, filed on Nov. 27, 2001.

(51) Int. Cl. C07K 14/47; C12Q 1/68; C07H 21/04; C12P 21/02; C12N 5/06

(52) U.S. Cl. 530/350; 435/6; 435/69.1; 435/320.1; 435/325; 536/23.5

(57) ABSTRACT

The invention provides polynucleotides and polypeptides corresponding to novel gene sequences associated with the incidence of liver disease, and resistance to statin drugs, particularly pravastatin. The invention also provides polynucleotide fragments corresponding to the genomic and/or coding regions of these genes which comprise at least one polymorphic site per fragment. Allele-specific primers and probes which hybridize to these regions, and/or which comprise at least one polymorphic site are also provided. The polynucleotides, primers, and probes of the present invention are useful in phenotype correlations, paternity testing, medicine, and genetic analysis. Also provided are vectors, host cells, antibodies, and recombinant and synthetic methods for producing said polypeptides. The invention further relates to diagnostic and therapeutic methods for applying these novel polypeptides to the diagnosis, treatment, and/or prevention of various, diseases and/or disorders, particularly hepatic and cardiovascular diseases related to these polypeptides, such as liver disease and high cholesterol. The invention further relates to screening methods for identifying agonists and antagonists of the polynucleotides and polypeptides of the present invention.

This application claims benefit to provisional application U.S. Serial No. 60/324,172 filed Sep. 21, 2001; and to provisional application U.S. Serial No. 60/333,700 filed Nov. 27, 2001. The entire teachings of the referenced applications are incorporated herein by reference.

### FIELD OF THE INVENTION

The invention provides polynucleotides and polypeptides corresponding to novel gene sequences associated with the incidence of liver disease, and resistance to statin drugs, particularly pravastatin. The invention also provides polynucleotide fragments corresponding to the genomic and/or coding regions of these genes which comprise at least one polymorphic site per fragment. Allele-specific primers and probes which hybridize to these regions, and/or which comprise at least one polymorphic site are also provided. The polynucleotides, primers, and probes of the present invention are useful in phenotype correlations, paternity testing, medicine, and genetic analysis. Also provided are vectors, host cells, antibodies, and recombinant and synthetic methods for producing said polypeptides. The invention further relates to diagnostic and therapeutic methods for applying these novel polypeptides to the diagnosis, treatment, and/or prevention of various diseases and/or disorders, particularly hepatic and cardiovascular diseases related to these polypeptides, such as liver disease and high cholesterol. The invention further relates to screening methods for identifying agonists and antagonists of the polynucleotides and polypeptides of the present invention.

### BACKGROUND OF THE INVENTION

The genomes of all organisms undergo spontaneous mutation in the course of their continuing evolution, generating variant forms of progenitor nucleic acid sequences (Guselia, Ann. Rev. Biochem., 55:831-854 (1986). The variant form may confer an evolutionary advantage or disadvantage relative to a progenitor form, or may be neutral. In some instances, a variant form confers a lethal disadvantage and is not transmitted to subsequent generations of the organism. In other instances, a variant form confers an evolutionary advantage to the species and is eventually incorporated into the DNA of many or most members of the species and effectively becomes the progenitor form. In many instances, both progenitor and variant form(s) survive and co-exist in a species population. The coexistence of multiple forms of a sequence gives rise to polymorphisms.

Several different types of polymorphism have been reported. A restriction fragment length polymorphism (RFLP) is a variation in DNA sequence that alters the length of a restriction fragment (Botstein et al., Am. J. Hum. Genet, 32:314-331 (1980). The restriction fragment length polymorphism may create or delete a restriction site, thus changing the length of the restriction fragment. RFLPs have been widely used in human and animal genetic analyses (see WO 90/13668; W090/11369; Donis-Keller, Cell , 51:319-337 (1987); Lander et al., Genetics 121,85-99 (1989)). When a heritable trait can be linked to a particular RFLP, the presence of the RFLP in an individual can be used to predict the likelihood that the animal will also exhibit the trait.

Other polymorphisms take the form of short tandem repeats (STRs) that include tandem di-, tri- and tetra-nucleotide repeated motifs. These tandem repeats are also referred to as variable number tandem repeat (VNTR) polymorphisms. VNTRs have been used in identity and paternity analysis (U.S. Pat. No. 5,075,217; Annour et al., FEBSLett. 307, 113-115 (1992); Horn et al., WO 91/14003; Jeffreys, EP 370,719), and in a large number of genetic mapping studies.

Other polymorphisms take the form of single nucleotide variations between individuals of the same species. Such polymorphisms are far more frequent than RFLPs, STRs and VNTRs. Some single nucleotide polymorphisms (SNP) occur in protein-coding nucleic acid sequences (coding sequence SNP (cSNP)), in which case, one of the polymorphic forms may give rise to the expression of a defective or otherwise variant protein and, potentially, a genetic disease. Examples of genes in which polymorphisms within coding sequences give rise to genetic disease include˜globin (sickle cell anemia), apoE4 (Alzheimer's Disease), Factor V Leiden (thrombosis), and CFTR (cystic fibrosis). cSNPs can alter the codon sequence of the gene and therefore specify an alternative amino acid. Such changes are called “missense” when another amino acid is substituted, and “nonsense” when the alternative codon specifies a stop signal in protein translation. When the cSNP does not alter the amino acid specified the cSNP is called “silent”.

Other single nucleotide polymorphisms occur in noncoding regions. Some of these polymorphisms may also result in defective protein expression (e.g., as a result of defective splicing). Other single nucleotide polymorphisms have no phenotypic effects. Single nucleotide polymorphisms can be used in the same manner as RFLPs and VNTRs, but offer several advantages.

Single nucleotide polymorphisms occur with greater frequency and are spaced more uniformly throughout the genome than other forms of polymorphism. The greater frequency and uniformity of single nucleotide polymorphisms means that there is a greater probability that such a polymorphism will be found in close proximity to a genetic locus of interest than would be the case for other polymorphisms. The different forms of characterized single nucleotide polymorphisms are often easier to distinguish than other types of polymorphism (e.g., by use of assays employing allele-specific hybridization probes or primers).

Only a small percentage of the total repository of polymorphisms in humans and other organisms has been identified. The limited number of polymorphisms identified to date is due to the large amount of work required for their detection by conventional methods. For example, a conventional approach to identifying polymorphisms might be to sequence the same stretch of DNA in a population of individuals by dideoxy sequencing. In this type of approach, the amount of work increases in proportion to both the length of sequence and the number of individuals in a population and becomes impractical for large stretches of DNA or large numbers of persons.

The liver functions in the clearance of a large variety of metabolic products, drugs and other xenobiotics by transporting them from the circulation into hepatocytes and then from the hepatocyte into the bile. Compounds must first cross the sinusoidal or basolateral membrane and, on the opposite side of the cell, must cross the canilicular membrane into the bile. Several classes of transport systems have been described that mediate the sinusoidal transport processes including the Na+/taurocholate cotransporter polypeptide, NTCP, in rat and human liver (Hagenbuch, B., et al. (1991) Proc. Natl. Acad. Sci. USA 88:10629-33; Hagenbuch, B. et al., (1994) J. Clin. Invest. 93:1326-31) and a family of organic anion transporting polypeptides (OATPs) that are principally expressed in liver, kidney and brain, and transport a broad spectrum of substrates in a sodium-independent manner (Meier, P. J., et al., (1997) Hepatology 26:1667-77; Wolkoff, A. W., (1996) Semin. Liver Dis. 16:121-127). The distribution of this latter family of transporters in liver, kidney and choroid plexus in the brain is thought to reflect common physiological requirements of these organs for the clearance of a multitide of organic anions. OATP isoforms identified in the rat include roatp1 (Jacquemin, E., et al., (1994) Proc. Natl. Acad. Sci. USA 91:133-37); roatp2 (Noe, B. A., et al., (1997) Proc. Natl. Acad. Sci. USA 94:10346-50; and roatp3 (Abe, T., et al., (1998) J. Biol. Chem. 273:11395-401). A total of 5 human OATPs, with documented activity in transfection experiments, have been described. Three of these have been characterized with respect to substrate specificities, tissue distribution and cellular localization. They include OATP1 (Kullak-Ublick, G. A., et al., (1995) Gastroenterology, 109:1274-1282), OATP2/LST1 (Hsiang, B. et al., (1999) J. Biol. Chem., 274:37161-37168; Abe, T. et al., (1999) J. Biol. Chem., 274:17159-17163), and OATP8/LST-2 (Konig, J. et al. (2000) J. Biol. Chem., 275: 23161-23168; Abe, T. et al., (2001) Gastroenterology, 120:1689-1699).

In addition to bile acids, OATPs are known to transport a variety of other endogenous substances. The known endogenous substrates of OATP2 are cholate, taurocholate, thyroid hormones T3 and T4, DHEAS, estradiol-17beta-glucuronide, estrone-3-sulfate, prostaglandin E2, thromboxane B2, leukotriene C4, leukotriene E4, and bilirubin and its conjugates. Known drugs that are transported by OATP2 include pravastatin, simvastatin, atorvastatin, and lovastatin (Hsiang, B. et al., (1999) J. Biol. Chem., 274:37161-37168; Abe, T. et al., (1999) J. Biol. Chem., 274:17159-17163; Konig, J. et al., (2000) Am. J. Physiol., 278:G156-G164; Cui, Y. et al., (2001) J. Biol. Chem. 276:9626-9630)

A number of transporters of the multi-drug resistant protein, or MRP, class have been characterized that mediate the transport of endogenous substances and drugs out of cells. Their principal substrates are lipophillic substances conjugated to glutathione, glucuronate, or sulfate, although non-conjugated compounds are also known to be transported. This is a therapeutically important protein family since MRPs confer resistance to drugs due to their ability to rid cells of xenobiotics. In the case of chemotherapeutics, they limit the efficacy of anti-cancer drugs. In other cases they provide a route of drug elimination from the body. An example of the latter is the cMOAT (also known as MRP2, cMRP and ABCC2) mediated transport of pravastatin across the canilicular membrane of the hepatocyte and out of the liver (Masayo, Y. et al., (1997) Drug Metab Dispos., 25:1123-1129). Many other substrates of cMOAT are known including leukotriene C4, leukotriene D4, leukotriene E4, conjugated bilirubin, 17β-glucuronosyl estradiol, ochratoxin A, glucuronosyl nafenopin, glucuronosyl grepafloxacin, and temocaprilat (for reviews of the MRP family and subsrate specificities see Koenig, J. et al.,(1999) Biochimica et Biophysica Acta, 1461:377-394; Borst, P. et al., (1999) Biochimica et Biophysica Acta, 1461:347-357). Chemotherapeutic drugs that have been identified as substrates for cMOAT include methotrexate, doxirubicin, cisplatin, CPT-11, SN-38, vincristine, and etoposide (Kool, M., et al., (1997) Cancer Res. 57:3537-3547; Koike, K. et al., (1997 Cancer Res. 57: 5475-5479; Cui, Y. et al., (1999) Mol. Pharmacol. 55:929-937).

Lipid-lowering drugs, in particular statin treatments, have been shown to reduce the incidence of initial and recurrent coronary heart disease (CHD) events within several years of initiating therapy. This effect can be clinically detected within the first 1 to 2 years in randomized trials.

Recent observational and clinical trial data suggest that lipid-lowering therapy initiated at the time of an acute coronary event can reduce recurrent events, and possibly all-cause mortality, in a much shorter period of time. The possible mechanisms by which this benefit occurs include the effect of reduced lipoprotein levels, as well as an independent effect of statins on endothelial function. Statins improve endothelial-dependent flow-mediated vasodilation by increasing the bioavailability of nitric oxide. Statins have also been shown to stabilize and decrease the formation of arterial plaques by modulating the inflammatory response within the vessel wall.

Polymorphisms in the human OATP2 gene may cause alterations in OATP2 expression and/or activity and, consequently, affect the rate of transport of its substrates into the liver. Known substrates for OATP2 are (1) the bile acid taurocholate, (2) thyroid hormones (thyroxine & triiodothyronine), (3) DHEAS, (4) estradiol-17β-glucuronide, (5) estrone-3-sulfate, (6) prostaglandin E2, (7) thromboxane B2, (8) leukotriene C4, (9) leukotiene E4, (10) bilirubin and its glucuronate conjugates, and (11) HMG-CoA reductase inhibitors including pravastatin, simvastatin, lovastatin, and atorvastatin. Thus, such polymorphisms may genetically predispose certain individuals to an increased risk of adverse consequences from the enhanced or impaired hepatic uptake of these substances. This could include, for example, drug or xenobiotic induced cholestasis due to decreased bile acid uptake, and hyperbilirubinemia due to the decreased uptake of conjugated and unconjugated bilirubin,. In addition, polymorphisms that result in decreased OATP2 levels could also pre-dispose patients to decreased responses to cholesterol lowering drugs (statins) such as pravastatin, simvastatin, lovastatin, pitivastatin, cerivastatin, and rousuvastatin. Such polymorphisms are expected to show a significant difference in allele frequency between healthy individuals and diseased (e.g. cholestatic) subjects or between drug (e.g. pravastatin) responsive and non-responsive subjects.

Polymorphisms in the human cMOAT gene may cause alterations in MRP2 expression and/or activity and, consequently, affect the rate of transport of its substrates out of cells. The cells or tissues that cMOAT resides include the liver, kidney, and intestine and in a variety of tumor cells. The latter develop so-called MDR drug resistance to cytotoxic anti-cancer drugs by virture of upregulating the expression of cMOAT. Such polymorphisms may genetically predispose certain individuals to an increased risk of adverse consequences from the enhanced or impaired uptake of cMOAT substrates. This could include, for example, drug or xenobiotic induced cholestasis due to decreased bile acid transport out of the liver, and hyperbilirubinemia due to the decreased hepatic transport of conjugated and unconjugated bilirubin. In addition, polymorphisms that result in increased or decreased cMOAT levels could also pre-dispose patients to atypical responses to cholesterol lowering drugs (statins) such as pravastatin. For example, patients with cMOAT polymorphisms may have enhanced or diminished export of statins from the liver and, thus have decreased or increased plasma cholesterol lowering responses to these drugs. Similarly, patients with polymorphisms in the human cMOAT gene may also exhibit atypical responses to cytotoxic anti-cancer therapy. For example, polymorphisms that enhance its activity may respond less well to drugs that are substrates for cMOAT.

### SUMMARY OF THE INVENTION

The present invention relates to the identification of polymorphisms which can predispose individuals to disease, by resequencing large numbers of genes in a large number of individuals. Various genes from a number of individuals have been resequenced as described herein, and SNPs in these genes have been discovered (see Tables I, IV, V, or VI). Some of these SNPs are cSNPs which specify a different amino acid sequence (described as “missense” under the ‘Mutation Type’ column of Tables IV, V, or VI); some of the SNPs are silent cSNPs (shown as mutation type “silent” under the ‘Mutation Type’ column of Tables IV, V, or VI), and some of these cSNPs may specify a stop signal in protein translation. Some of the identified SNPs were located in non-coding regions (described as “non-CDS” in the ‘Mutation Type’ column of Tables IV, V, or VI).

The invention relates to a nucleic acid molecule which comprises a single nucleotide polymorphism at a specific location. In a particular embodiment the invention relates to the variant allele of a gene having a single nucleotide polymorphism, which variant allele differs from a reference allele by one nucleotide at the site(s) identified in Tables I, IV, V, or VI. Complements of these nucleic acid segments are also provided. The segments can be DNA or RNA, and can be double- or single-stranded. Segments can be, for example, 5-10,5-15, 10-20,5-25,10-30, 10-50 or 10-100 bases long.

The invention further provides allele-specific oligonucleotides that hybridize to a nucleic acid molecule comprising a single nucleotide polymorphism or to the complement of the nucleic acid molecule. These oligonucleotides can be probes or primers.

The invention further provides oligonucleotides that may be used to amplify across a single nucleotide polymorphic site of the present invention. The invention further provides oligonucleotides that may be used to sequence said amplified sequence. The invention further provides a method of analyzing a nucleic acid from a DNA sample using said amplification and sequencing primers to assess whether said sample contains the reference or variant base (allele) at the polymorphic site, comprising the steps of amplifying a sequence using appropriate PCR primers for amplifying across a polymorphic site, sequencing the resulting amplified product using appropriate sequencing primers to sequence said product, and determining whether the variant or reference base is present at the polymorphic site.

The invention further provides a method of analyzing a nucleic acid from DNA sample(s) from various ethnic populations using said amplification and sequencing primers to assess whether said sample(s) contain the reference or variant base (allele) at the polymorphic site in an effort to identify individuals with low hepatic statin uptake comprising the steps of amplifying a sequence using appropriate PCR primers for amplifying across a polymorphic site, sequencing the resulting amplified product using appropriate sequencing primers to sequence said product, and determining whether the variant or reference base is present at the polymorphic site, and optionally determining the statistical association between either the reference or variant allele at the polymorphic site(s) to the incidence low hepatic statin uptake.

The invention further provides a method of analyzing a nucleic acid from DNA sample(s) from various ethnic populations using said amplification and sequencing primers to assess whether said sample(s) contain the reference or variant base (allele) at the polymorphic site in an effort to identify individuals most likely to be non-responsive, or less responsive, to statin administration comprising the steps of amplifying a sequence using appropriate PCR primers for amplifying across a polymorphic site, sequencing the resulting amplified product using appropriate sequencing primers to sequence said product, and determining whether the variant or reference base is present at the polymorphic site, and optionally determining the statistical association between either the reference or variant allele at the polymorphic site(s) to the incidence of non-responsive, or less responsive statin responses.

The invention further provides oligonucleotides that may be used to genotype DNA sample(s) to assess whether said sample(s) contain the reference or variant base (allele) at the polymorphic site(s). The invention provide a method of using oligonucleotides that may be used to genotype a DNA sample to assess whether said sample contains the reference or variant base (allele) at the polymorphic site comprising the steps of amplifying a sequence using appropriate PCR primers for amplifying across a polymorphic site, subjecting the product of said amplification to a genetic bit analysis (GBA) reaction, and analyzing the result.

The invention provides a method of using oligonucleotides that may be used to genotype DNA sample(s) to identify individual(s) that may be at risk of developing drug interactions upon administration of at least one statin, or other drug, to assess whether said sample(s) contains the reference or variant base (allele) at the polymorphic site(s) comprising the steps of amplifying a sequence using appropriate PCR primers for amplifying across a polymorphic site, subjecting the product of said amplification to a genetic bit analysis (GBA) reaction, analyzing the result, and optionally determining the statistical association between either the reference or variant allele at the polymorphic site(s) to the incidence of said drug intereaction.

The invention provides a method of using oligonucleotides that may be used to genotype DNA sample(s) to identify ethnic population(s) that may be at risk of developing drug interactions upon administration of at least one statin, or other drug, to assess whether said sample(s) contain the reference or variant base (allele) at the polymorphic site comprising the steps of amplifying a sequence using appropriate PCR primers for amplifying across a polymorphic site, subjecting the product of said amplification to a genetic bit analysis (GBA) reaction, analyzing the result, and optionally determining the statistical association between either the reference or variant allele at the polymorphic site(s) to the incidence of said drug interaction.

The invention further provides a method of analyzing a nucleic acid from an individual. The method allows the determination of whether the reference or variant base is present at any one, or more, of the polymorphic sites shown in Tables I, IV, V, or VI. Optionally, a set of bases occupying a set of the polymorphic sites shown in Tables I, IV, V, or VI is determined. This type of analysis can be performed on a number of individuals, who are also tested (previously, concurrently or subsequently) for the presence of a disease phenotype. The presence or absence of disease phenotype is then correlated with a base or set of bases present at the polymorphic site or sites in the individuals tested.

Thus, the invention further relates to a method of predicting the presence, absence, likelihood of the presence or absence, or severity of a particular phenotype or disorder associated with a particular genotype. The method comprises obtaining a nucleic acid sample from an individual and determining the identity of one or more bases (nucleotides) at specific (e.g., polymorphic) sites of nucleic acid molecules described herein, wherein the presence of a particular base at that site is correlated with a specified phenotype or disorder, thereby predicting the presence, absence, likelihood of the presence: or absence, or severity of the phenotype or disorder in the individual.

The present invention also relates to recombinant vectors, which include the isolated nucleic acid molecules of the present invention, and to host cells containing the recombinant vectors, as well as to methods of making such vectors and host cells, in addition to their use in the production of polypeptides or peptides provided herein using recombinant techniques. Synthetic methods for producing the polypeptides and polynucleotides of the present invention are provided. Also provided are diagnostic methods for detecting diseases, disorders, and/or conditions related to the polypeptides and polynucleotides provided herein, and therapeutic methods for treating such diseases, disorders, and/or conditions. The invention further relates to screening methods for identifying binding partners of the polypeptides.

The invention further provides an isolated polypeptide having an amino acid sequence encoded by a polynucleotide described herein.

The invention further relates to a method for genotyping an individual comprising the steps of (a) obtaining a nucleic acid sample(s) from said individual; (b) determining the nucleotide present at least one polymorphic position, and (c) comparing said at least one polymorphic position with a known data set.

The invention further relates to a method for genotyping an individual comprising the steps of (a) obtaining a nucleic acid sample(s) from said individual; and (b) determining the nucleotide present at least one polymorphic position, wherein the nucleotide present at the at least one polmorphic position is associated with a specific disease, disorder, and/or condition as described herein.

### BRIEF DESCRIPTION OF THE FIGURES/DRAWINGS

FIGS. 1A-C show the polynucleotide sequence (SEQ ID NO:1) and deduced amino acid sequence (SEQ ID NO:2) of the wild type human organic anion transport protein, OATP2, also referred to as solute carrier family 21 member 6 (HGNC NO:SLC21A6; Genbank Accession No: gi|6636521). The standard one-letter abbreviation for amino acids is used to illustrate the deduced amino acid sequence. The polynucleotide sequence contains a sequence of 2830 nucleotides (SEQ ID NO:1), encoding a polypeptide of 691 amino acids (SEQ ID NO:2).

FIGS. 2A-F show the polynucleotide sequence (SEQ ID NO:3) and deduced amino acid sequence (SEQ ID NO:2) of the wild type human organic anion transport protein, cMOAT, also referred to as ATP-binding cassette sub-family C member 2 (HGNC NO: ABCC2; Genbank Accession No: gi|1574997). The standard one-letter abbreviation for amino acids is used to illustrate the deduced amino acid sequence. The polynucleotide sequence contains a sequence of 5300 nucleotides (SEQ ID NO:3), encoding a polypeptide of 1545 amino acids (SEQ ID NO:4).

FIGS. 3A-C show the polynucleotide sequence (SEQ ID NO:5) and deduced amino acid sequence (SEQ ID NO:6) of the human organic anion transport OATP2 protein variant, SLC21A6-S137S (SNP_ID: PS100s1) of the present invention. The standard one-letter abbreviation for amino acids is used to illustrate the deduced amino acid sequence. The polynucleotide sequence contains a sequence of 2830 nucleotides (SEQ ID NO:5), encoding a polypeptide of 691 amino acids (SEQ ID NO:6). The predicted ‘G’ to ‘A’ polynucleotide polymorphism is located at nucleic acid position 545 of SEQ ID NO:5 and is represented in bold. The polymorphism is a silent mutation and does not change the amino acid sequence of the encoded polypeptide.

FIGS. 4A-C show the polynucleotide sequence (SEQ ID NO:7) and deduced amino acid sequence (SEQ ID NO:8) of the human organic anion transport OATP2 protein variant, SLC21A6-P155T (SNP_ID: PS100s2) of the present invention. The standard one-letter abbreviation for amino acids is used to illustrate the deduced amino acid sequence. The polynucleotide sequence contains a sequence of 2830 nucleotides (SEQ ID NO:7), encoding a polypeptide of 691 amino acids (SEQ ID NO:8). The predicted ‘C’ to ‘A’ polynucleotide polymorphism is located at nucleic acid position 597 of SEQ ID NO:7 and is represented in bold. The polymorphism is a missense mutation resulting in a change in an encoding amino acid from ‘P’ to ‘T’ at amino acid position 155 of SEQ ID NO:8 and is represented by underlining.

FIGS. 5A-C show the polynucleotide sequence (SEQ ID NO:9) and deduced amino acid sequence (SEQ ID NO: 10) of the human organic anion transport OATP2 protein variant, SLC21A6-D130Y (SNP_ID: PS100s9) of the present invention. The standard one-letter abbreviation for amino acids is used to illustrate the deduced amino acid sequence. The polynucleotide sequence contains a sequence of 2830 nucleotides (SEQ ID NO:9), encoding a polypeptide of 691 amino acids (SEQ ID NO:10). The predicted ‘G’ to ‘T’ polynucleotide polymorphism is located at nucleic acid position 522 of SEQ ID NO:9 and is represented in bold. The polymorphism is a missense mutation resulting in a change in an encoding amino acid from ‘D’ to ‘Y’ at amino acid position 130 of SEQ ID NO:10 and is represented by underlining.

FIGS. 6A-C show the polynucleotide sequence (SEQ ID NO:11) and deduced amino acid sequence (SEQ ID NO:12) of the human organic anion transport OATP2 protein variant, SLC21A6-G488A (SNP_ID: PS100s23) of the present invention. The standard one-letter abbreviation for amino acids is used to illustrate the deduced amino acid sequence. The polynucleotide sequence contains a sequence of 2830 nucleotides (SEQ ID NO:11), encoding a polypeptide of 691 amino acids (SEQ ID NO:12). The predicted ‘G’ to ‘C’ polynucleotide polymorphism is located at nucleic acid position 1597 of SEQ ID NO:11 and is represented in bold. The polymorphism is a missense mutation resulting in a change in an encoding amino acid from ‘G’ to ‘A’ at amino acid position 488 of SEQ ID NO:12 and is represented by underlining.

FIGS. 7A-C show the polynucleotide sequence (SEQ ID NO:13) and deduced amino acid sequence (SEQ ID NO:14) of the human organic anion transport OATP2 protein variant, SLC21A6-V416V (SNP_ID: PS100s25) of the present invention. The standard one-letter abbreviation for amino acids is used to illustrate the deduced amino acid sequence. The polynucleotide sequence contains a sequence of 2830 nucleotides (SEQ ID NO:13), encoding a polypeptide of 691 amino acids (SEQ ID NO:14). The predicted ‘G’ to ‘A’ polynucleotide polymorphism is located at nucleic acid position 1382 of SEQ ID NO:13 and is represented in bold. The polymorphism is a silent mutation and does not change the amino acid sequence of the encoded polypeptide.

FIGS. 8A-C show the polynucleotide sequence (SEQ ID NO:15) and deduced amino acid sequence (SEQ ID NO:16) of the human organic anion transport OATP2 protein variant, SLC21A6-F400K (SNP_ID: PS100s26) of the present invention. The standard one-letter abbreviation for amino acids is used to illustrate the deduced amino, acid sequence. The polynucleotide sequence contains a sequence of 2830 nucleotides (SEQ ID NO:15), encoding a polypeptide of 691 amino acids (SEQ ID NO:16). The predicted ‘C’ to ‘G’ polynucleotide polymorphism is located at nucleic acid position 1334 of SEQ ID NO:15 and is represented in bold. The polymorphism is a missense mutation resulting in a change in an encoding amino acid from ‘F’ to ‘K’ at amino acid position 400 of SEQ ID NO:16 and is represented by underlining.

FIGS. 9A-C show the polynucleotide sequence (SEQ ID NO:17) and deduced amino acid sequence (SEQ ID NO:18) of the human organic anion transport OATP2 protein variant, SLC21A6-V174A (SNP_ID: PS100s29) of the present invention. The standard one-letter abbreviation for amino acids is used to illustrate the deduced amino acid sequence. The polynucleotide sequence contains a sequence of 2830 nucleotides (SEQ ID NO:17), encoding a polypeptide of 691 amino acids (SEQ ID NO:18). The predicted ‘T’ to ‘C’ polynucleotide polymorphism is located at nucleic acid position 655 of SEQ ID NO:17 and is represented in bold. The polymorphism is a missense mutation resulting in a change in an encoding amino acid from ‘V’ to ‘A’ at amino acid position 174 of SEQ ID NO:18 and is represented by underlining.

FIGS. 10A-C show the polynucleotide sequence (SEQ ID NO:19) and deduced amino acid sequence (SEQ ID NO:20) of the human organic anion transport OATP2 protein variant, SLC21A6-K191L (SNP_ID: PS100s30) of the present invention. The standard one-letter abbreviation for amino acids is used to illustrate the deduced amino acid sequence. The polynucleotide sequence contains a sequence of 2830 nucleotides (SEQ ID NO:19), encoding a polypeptide of 691 amino acids (SEQ ID NO:20). The predicted ‘T’ to ‘C’ polynucleotide polymorphism is located at nucleic acid position 705 of SEQ ID NO:19 and is represented in bold. The polymorphism is a missense mutation resulting in a change in an encoding amino acid from ‘K’ to ‘L’ at amino acid position 191 of SEQ ID NO:20 and is represented by underlining.

FIGS. 11A-C show the polynucleotide sequence (SEQ ID NO:21) and deduced amino acid sequence (SEQ ID NO:22) of the human organic anion transport OATP2 protein variant, SLC21A6-F199F (SNP_ID: PS100s31) of the present invention. The standard one-letter abbreviation for amino acids is used to illustrate the deduced amino acid sequence. The polynucleotide sequence contains a sequence of 2830 nucleotides (SEQ ID NO:21), encoding a polypeptide of 691 amino acids (SEQ ID NO:22). The predicted ‘C’ to ‘T’ polynucleotide polymorphism is located at nucleic acid position 731 of SEQ ID NO:21 and is represented in bold. The polymorphism is a silent mutation and does not change the amino acid sequence of the encoded polypeptide.

FIGS. 12A-F show the polynucleotide sequence (SEQ ID NO:23) and deduced amino acid sequence (SEQ ID NO:24) of the human organic anion transport cMOAT protein variant, ABCC2-E1188V (SNP_ID: PS101s1) of the present invention. The standard one-letter abbreviation for amino acids is used to illustrate the deduced amino acid sequence. The polynucleotide sequence contains a sequence of 5300 nucleotides (SEQ ID NO:23), encoding a polypeptide of 1545 amino acids (SEQ ID NO:24). The predicted ‘A’ to ‘T’ polynucleotide polymorphism is located at nucleic acid position 3664 of SEQ ID NO:23 and is represented in bold. The polymorphism is a missense mutation resulting in a change in an encoding amino acid from ‘E’ to ‘V’ at amino acid position 1188 of SEQ ID NO:24 and is represented by underlining.

FIGS. 13A-F show the polynucleotide sequence (SEQ ID NO:25) and deduced amino acid sequence (SEQ ID NO:26) of the human organic anion transport cMOAT protein variant, ABCC2-I13241 (SNP_ID: PS101s2) of the present invention. The standard one-letter abbreviation for amino acids is used to illustrate the deduced amino acid sequence. The polynucleotide sequence contains a sequence of 5300 nucleotides (SEQ ID NO:25), encoding a polypeptide of 1545 amino acids (SEQ ID NO:26). The predicted ‘C’ to ‘T’ polynucleotide polymorphism is located at nucleic acid position 4073 of SEQ ID NO:25 and is represented in bold. The polymorphism is a silent mutation and does not change the amino acid sequence of the encoded polypeptide.

FIGS. 14A-F show the polynucleotide sequence (SEQ ID NO:27) and deduced amino acid sequence (SEQ ID NO:28) of the human organic anion transport cMOAT protein variant, ABCC2-L1370L (SNP_ID: PS101s4) of the present invention. The standard one-letter abbreviation for amino acids is used to illustrate the deduced amino acid sequence. The polynucleotide sequence contains a sequence of 5300 nucleotides (SEQ ID NO:27), encoding a polypeptide of 1545 amino acids (SEQ ID NO:28). The predicted ‘C’ to ‘T’ polynucleotide polymorphism is located at nucleic acid position 4211 of SEQ ID NO:27 and is represented in bold. The polymorphism is a silent mutation and does not change the amino acid sequence of the encoded polypeptide.

FIGS. 15A-F show the polynucleotide sequence (SEQ ID NO:29) and deduced amino acid sequence (SEQ ID NO:30) of the human organic anion transport cMOAT protein variant, ABCC2-A1354A (SNP_ID: PS101s5) of the present invention. The standard one-letter abbreviation for amino acids is used to illustrate the deduced amino acid sequence. The polynucleotide sequence contains a sequence of 5300 nucleotides (SEQ ID NO:29), encoding a polypeptide of 1545 amino acids (SEQ ID NO:30). The predicted ‘C’ to ‘T’ polynucleotide polymorphism is located at nucleic acid position 4163 of SEQ ID NO:29 and is represented in bold. The polymorphism is a silent mutation and does not change the amino acid sequence of the encoded polypeptide.

FIG. 16A-F show the polynucleotide sequence (SEQ ID NO:31) and deduced amino acid sequence (SEQ ID NO:32) of the human organic anion transport cMOAT protein variant, ABCC2-E1470E (SNP_ID: PS101s6) of the present invention. The standard one-letter abbreviation for amino acids is used to illustrate the deduced amino acid sequence. The polynucleotide sequence contains a sequence of 5300 nucleotides (SEQ ID NO:31), encoding a polypeptide of 1545 amino acids (SEQ ID NO:32). The predicted ‘G’ to ‘A’ polynucleotide polymorphism is located at nucleic acid position 4511 of SEQ ID NO:31 and is represented in bold. The polymorphism is a silent mutation and does not change the amino acid sequence of the encoded polypeptide.

FIG. 17A-F show the polynucleotide sequence (SEQ ID NO:33) and deduced amino acid sequence (SEQ ID NO:34) of the human organic anion transport cMOAT protein variant, ABCC2-H1496H (SNP_ID: PS101s7) of the present invention. The standard one-letter abbreviation for amino acids is used to illustrate the deduced amino acid sequence. The polynucleotide sequence contains a sequence of 5300 nucleotides (SEQ ID NO:33), encoding a polypeptide of 1545 amino acids (SEQ ID NO:34). The predicted ‘T’ to ‘C’ polynucleotide polymorphism is located at nucleic acid position 4589 of SEQ ID NO:33 and is represented in bold. The polymorphism is a silent mutation and does not change the amino acid sequence of the encoded polypeptide.

FIG. 18A-F show the polynucleotide sequence (SEQ ID NO:35) and deduced amino acid sequence (SEQ ID NO:36) of the human organic anion transport cMOAT protein variant, ABCC2-R1181L (SNP_ID: PS101s10) of the present invention. The standard one-letter abbreviation for amino acids is used to illustrate the deduced amino acid sequence. The polynucleotide sequence contains a sequence of 5300 nucleotides (SEQ ID NO:35), encoding a polypeptide of 1545 amino acids (SEQ ID NO:36). The predicted ‘G’ to ‘T’ polynucleotide polymorphism is located at nucleic acid position 3643 of SEQ ID NO:35 and is represented in bold. The polymorphism is a missense mutation resulting in a change in an encoding amino acid from ‘R’ to ‘L’ at amino acid position 1181 of SEQ ID NO:36 and is represented by underlining.

FIG. 19A-F show the polynucleotide sequence (SEQ ID NO:37) and deduced amino acid sequence (SEQ ID NO:38) of the human organic anion transport cMOAT protein variant, ABCC2-K961R (SNP_ID: PS101s11) of the present invention. The standard one-letter abbreviation for amino acids is used to illustrate the deduced amino acid sequence. The polynucleotide sequence contains a sequence of 5300 nucleotides (SEQ ID NO:37), encoding a polypeptide of 1545 amino acids (SEQ ID NO:38). The predicted ‘A’ to ‘G’ polynucleotide polymorphism is located at nucleic acid position 2983 of SEQ ID NO:37 and is represented in bold. The polymorphism is a missense mutation resulting in a change in an encoding amino acid from ‘K’ to ‘R’ at amino acid position 961 of SEQ ID NO:38 and is represented by underlining.

FIG. 20A-F show the polynucleotide sequence (SEQ ID NO:39) and deduced amino acid sequence (SEQ ID NO:40) of the human organic anion transport cMOAT protein variant, ABCC2-V86V (SNP_ID: PS101s13) of the present invention. The standard one-letter abbreviation for amino acids is used to illustrate the deduced amino acid sequence. The polynucleotide sequence contains a sequence of 5300 nucleotides (SEQ ID NO:39), encoding a polypeptide of 1545 amino acids (SEQ ID NO:40). The predicted ‘A’ to ‘G’ polynucleotide polymorphism is located at nucleic acid position 359 of SEQ ID NO:39 and is represented in bold. The polymorphism is a silent mutation and does not change the amino acid sequence of the encoded polypeptide.

FIGS. 21A-F show the polynucleotide sequence (SEQ ID NO:41) and deduced amino acid sequence (SEQ ID NO:42) of the human organic anion transport cMOAT protein variant, ABCC2-1670T (SNP_ID: PS101s22) of the present invention. The standard one-letter abbreviation for amino acids is used to illustrate the deduced amino acid sequence. The polynucleotide sequence contains a sequence of 5300 nucleotides (SEQ ID NO:41), encoding a polypeptide of 1545 amino acids (SEQ ID NO:42). The predicted ‘T’ to ‘C’ polynucleotide polymorphism is located at nucleic acid position 2110 of SEQ ID NO:41 and is represented in bold. The polymorphism is a missense mutation resulting in a change in an encoding amino acid from ‘I’ to ‘T’ at amino acid position 670 of SEQ ID NO:42 and is represented by underlining.

FIGS. 22A-F show the polynucleotide sequence (SEQ ID NO:43) and deduced amino acid sequence (SEQ ID NO:44) of the human organic anion transport cMOAT protein variant, ABCC2-V4171 (SNP_ID: PS101s23) of the present invention. The standard one-letter abbreviation for amino acids is used to illustrate the deduced amino acid sequence. The polynucleotide sequence contains a sequence of 5300 nucleotides (SEQ ID NO:43), encoding a polypeptide of 1545 amino acids (SEQ ID NO:44). The predicted ‘G’ to ‘A’ polynucleotide polymorphism is located at nucleic acid position 1350 of SEQ ID NO:43 and is represented in bold. The polymorphism is a missense mutation resulting in a change in an encoding amino acid from ‘V’ to ‘I’ at amino acid position 417 of SEQ ID NO:44 and is represented by underlining.

FIGS. 23A-F show the polynucleotide sequence (SEQ ID NO:45) and deduced amino acid sequence (SEQ ID NO:46) of the human organic anion transport cMOAT protein variant, ABCC2-L407K (SNP_ID: PS101s24) of the present invention. The standard one-letter abbreviation for amino acids is used to illustrate the deduced amino acid sequence. The polynucleotide sequence contains a sequence of 5300 nucleotides (SEQ ID NO:45), encoding a polypeptide of 1545 amino acids (SEQ ID NO:46). The predicted ‘C’ to ‘T’ polynucleotide polymorphism is located at nucleic acid position 1320 of SEQ ID NO:45 and is represented in bold. The polymorphism is a missense mutation resulting in a change in an encoding amino acid from ‘L’ to ‘K’ at amino acid position 407 of SEQ ID NO:46 and is represented by underlining.

FIG. 24A-F show the polynucleotide sequence (SEQ ID NO:47) and deduced amino acid sequence (SEQ ID NO:48) of the human organic anion transport cMOAT protein variant, ABCC2-S978S (SNP_ID: PS101s32) of the present invention. The standard one-letter abbreviation for amino acids is used to illustrate the deduced amino acid sequence. The polynucleotide sequence contains a sequence of 5300 nucleotides (SEQ ID NO:47), encoding a polypeptide of 1545 amino acids (SEQ ID NO:48). The predicted ‘G’ to ‘A’ polynucleotide polymorphism is located at nucleic acid position 3035 of SEQ ID NO:47 and is represented in bold. The polymorphism is a silent mutation and does not change the amino acid sequence of the encoded polypeptide.

FIGS. 25A-C show the polynucleotide sequence (SEQ ID NO:289) and deduced amino acid sequence (SEQ ID NO:290) of the human organic anion transport OATP2 protein comprising, or alternatively consisting of, one or more of the predicted polynucleotide polymorphic loci, in addition to, the encoded polypeptide polymorphic loci of the present invention for this particular protein, which include but are not limited to the following polynucleotide polymorphisms: SLC21A6-G545A (SNP_ID: PS100s1), SLC21A6-C597A (SNP_ID: PS100s2), SLC21A6-T522C (SNP_ID: PS100s9), SLC21A6-C1597G (SNP_ID: PS100s23), SLC21A6- G1382A (SNP_ID: PS100s25), SLC21A6- C1334G (SNP_ID: PS100s26), SLC21A6- T655C (SNP_ID: PS100s29), SLC21A6- T705C (SNP_ID: PS100s30) and/or SLC21A6- C731T (SNP_ID: PS100s31); and polypeptide polymorphisms—SLC21A6-P155T (SNP_ID: PS100s2), SLC21A6-D130Y (SNP_ID: PS100s9), SLC21A6-G488A (SNP_ID: PS100s23), SLC21A6-F400K (SNP_ID: PS100s26), and/or SLC21A6-V174A (SNP_ID: PS100s29). The standard one-letter abbreviation for amino acids is used to illustrate the deduced amino acid sequence. The polynucleotide sequence contains a sequence of 2830 nucleotides (SEQ ID NO:49), encoding a polypeptide of 691 amino acids (SEQ ID NO:50). The polynucleotide polymorphic sites are represented by an “N”, in bold. The polypeptide polymorphic sites are represented by an “X”, in bold. The present invention encompasses the polynucleotide at nucleotide position 545 as being either a “G” or an “A”, the polynucleotide at nucleotide position 597 as being either a “C” or an “A”, the polynucleotide at nucleotide position 522 as being either a “G” or a “T”, the polynucleotide at nucleotide position 1597 as being either a “G” or a “C”, the polynucleotide at nucleotide position 1382 as being either a “G” or an “A”, the polynucleotide at nucleotide position 1334 as being either a “C” or a “G”, the polynucleotide at nucleotide position 665 as being either a “T” or a “C”, the polynucleotide at nucleotide position 705 as being either a “T” or a “C”, and the polynucleotide at nucleotide position 731 as being either a “C” or a “T” of FIGS. 25A-C (SEQ ID NO:49), in addition to any combination thereof. The present invention also encompasses the polypeptide at amino acid position 155 as being either an “Pro” or an “Thr”, the polypeptide at amino acid position 130 as being either an “Asp” or a “Tyr”, the polypeptide at amino acid position 488 as being either an “Gly” or a “Ala”, the polypeptide at amino acid position 400 as being either an “Phe” or a “Lys”, and the polypeptide at amino acid position 174 as being either a “Val” or a “Ala” of FIGS. 25A-C (SEQ ID NO:50).

FIGS. 26A-F show the polynucleotide sequence (SEQ ID NO:289) and deduced amino acid sequence (SEQ ID NO:290) of the human organic anion transport cMOAT protein comprising, or alternatively consisting of, one or more of the predicted polynucleotide polymorphic loci, in addition to, the encoded polypeptide polymorphic loci of the present invention for this particular protein, which include but are not limited to the following polynucleotide polymorphisms: ABCC2- A3664T (SNP_ID: PS101s1), ABCC2- C4073T (SNP_ID: PS101s2), ABCC2- C4211T (SNP_ID: PS101s4), ABCC2- C4163T (SNP_ID: PS101s5), ABCC2- G4511A (SNP_ID: PS101s6), ABCC2- T4589C (SNP_ID: PS101s7), ABCC2- G3643T (SNP_ID: PS101s10), ABCC2- A2983G (SNP_ID: PS101s11), ABCC2- A359G (SNP_ID: PS101s13), ABCC2- T2110C (SNP_ID: PS101s22), ABCC2- G1350A (SNP_ID: PS101s23), ABCC2- C1320T (SNP_ID: PS101s24), and/or ABCC2- G3035A (SNP_ID: PS101s32); and polypeptide polymorphisms—ABCC2-E1188V (SNP_ID: PS101s1), ABCC2-R1181L (SNP_ID: PS101s10), ABCC2-K961R (SNP_ID: PS101s11), ABCC2-I670T (SNP_ID: PS101s22), ABCC2-V417I (SNP_ID: PS101s23), and/or ABCC2-L407K (SNP_ID: PS101s24). The standard one-letter abbreviation for amino acids is used to illustrate the deduced amino acid sequence. The polynucleotide sequence contains a sequence of 5300 nucleotides (SEQ ID NO:603), encoding a polypeptide of 1545 amino acids (SEQ ID NO:604). The polynucleotide polymorphic sites are represented by an “N”, in bold. The polypeptide polymorphic sites are represented by an “X”, in bold. The present invention encompasses the polynucleotide at nucleotide position 3664 as being either an “A” or a “T”, the polynucleotide at nucleotide position 4073 as being either a “C” or a “T”, the polynucleotide at nucleotide position 4211 as being either a “C” or a “T”, the polynucleotide at nucleotide position 4163 as being either a “C” or a “T”, the polynucleotide at nucleotide position 4511 as being either a “G” or an “A”, the polynucleotide at nucleotide position 4589 as being either a “T” or a “C”, the polynucleotide at nucleotide position 3643 as being either a “G” or a “T”, the polynucleotide at nucleotide position 2983 as being either an “A” or a “G”, the polynucleotide at nucleotide position 359 as being either an “A” or a “G”, the polynucleotide at nucleotide position 2110 as being either a “T” or a “C”, the polynucleotide at nucleotide position 1350 as being either a “G” or an “A”, the polynucleotide at nucleotide position 1320 as being either a “C” or a “T”, and the polynucleotide at nucleotide position 3035 as being either a “G” or an “A” of FIGS. 26A-F (SEQ ED NO:603), in addition to any combination thereof. The present invention also encompasses the polypeptide at amino acid position 1188 as being either an “Glu” or an “Val”, the polypeptide at amino acid position 1181 as being either an “Arg” or a “Leu”, the polypeptide at amino acid position 961 as being either an “Lys” or a “Arg”, the polypeptide at amino acid position 670 as being either an “Ile” or a “Thr”, the polypeptide at amino acid position 417 as being either an “Val” or a “Ile”, and the polypeptide at amino acid position 407 as being either a “Leu” or a “Lys” of FIGS. 26A-F (SEQ ID NO:604).

FIG. 27 illustrates an example of the possible haplotypes (A, B, C, and D) for an individual that has the following genotype at a particular genomic locus: A/G heterozygote at SNP1, G/C heterozygote at SNP2, and A/C heterozygote at SNP3.

FIG. 28 illustrates an example of how the haplotype of an individual at a particular genomic locus can be determined using a combination of the individuals genotype with the genotypes of the individuals parents genotypes at the same locus. The example is based upon one parent having an A/A genotype at SNP1, a G/C genotype at SNP2, and an A/A genotype at SNP3, and the other parent having an A/G genotype at SNP1, C/C genotype at SNP2, and C/C genotype at SNP3, and the child being heterozygote at all three SNPs. As shown, there is only one possible haplotype combination. The later is based upon the absence of a crossing over event at this locus during meiosis.

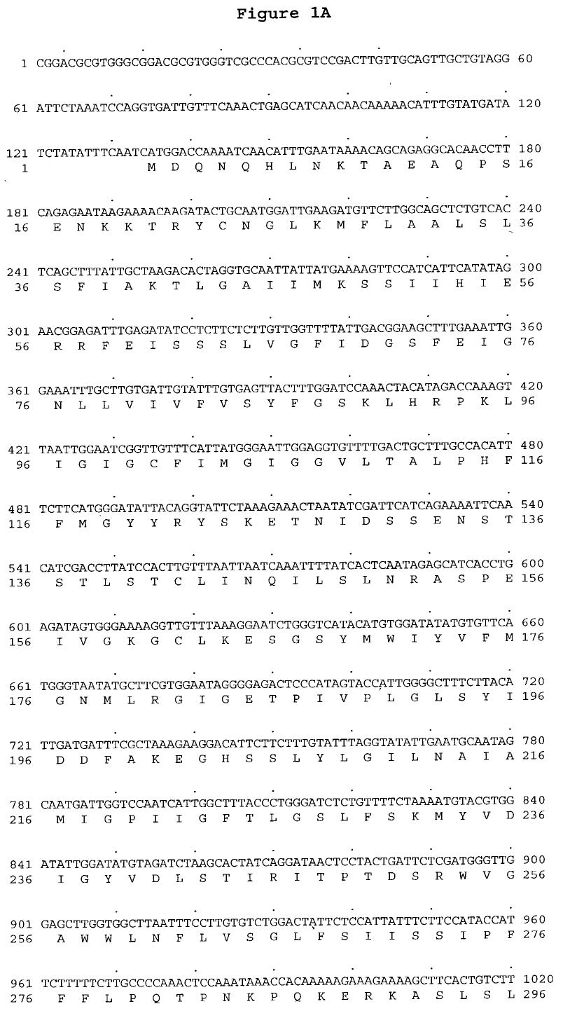

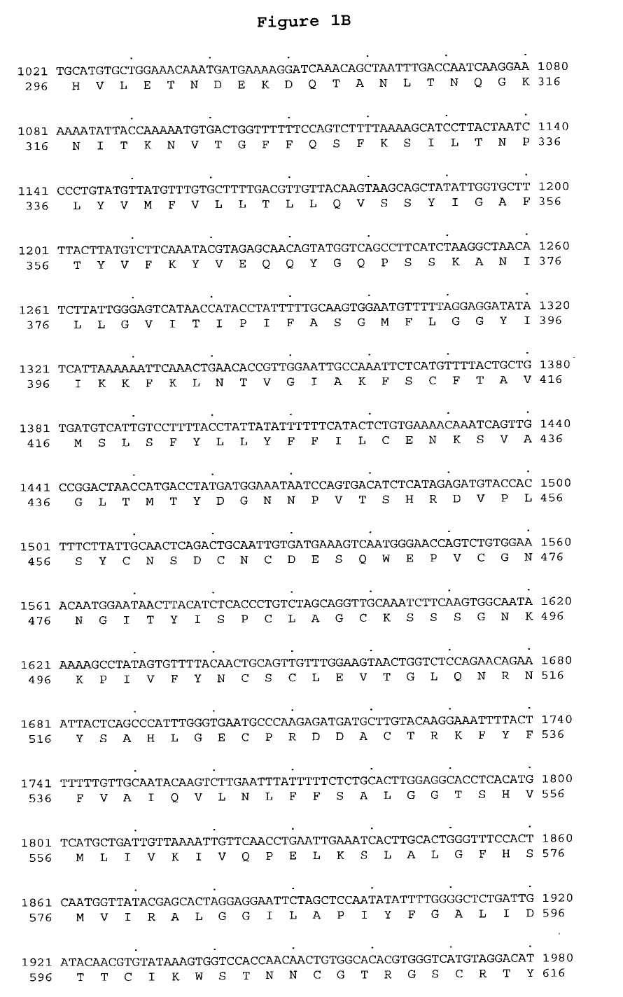

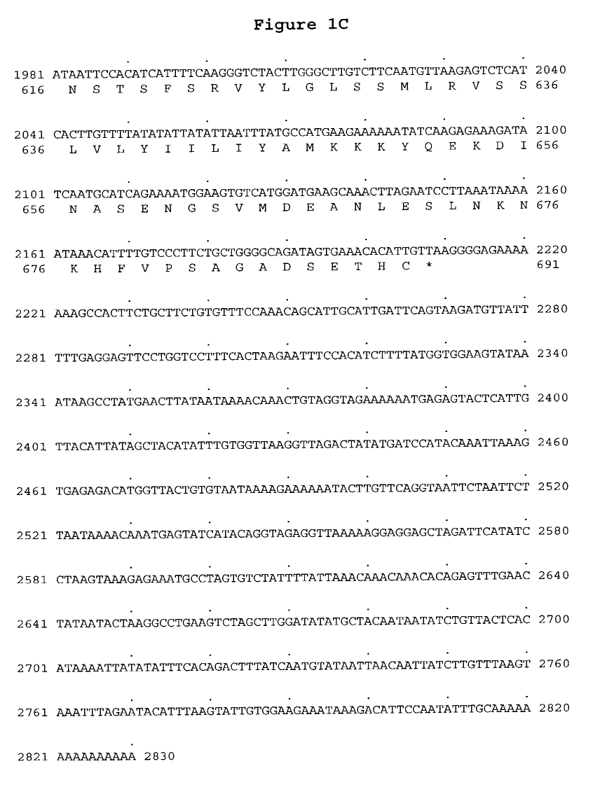

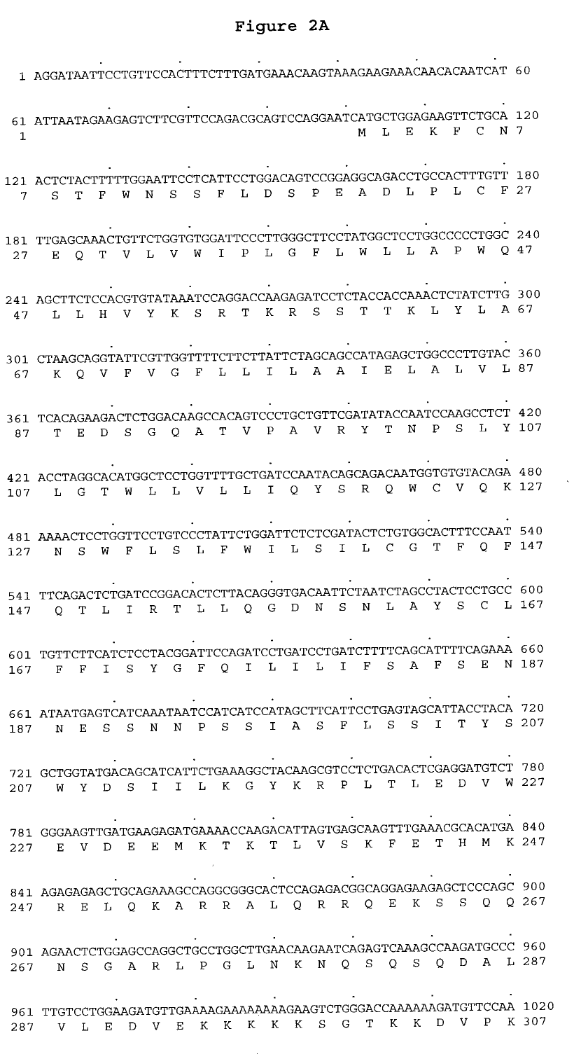

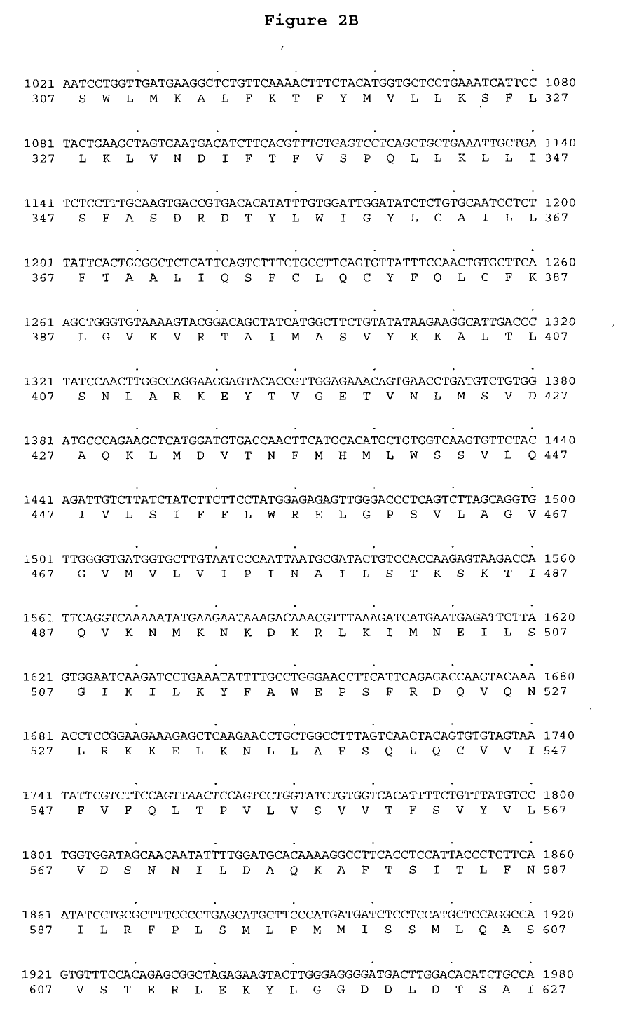

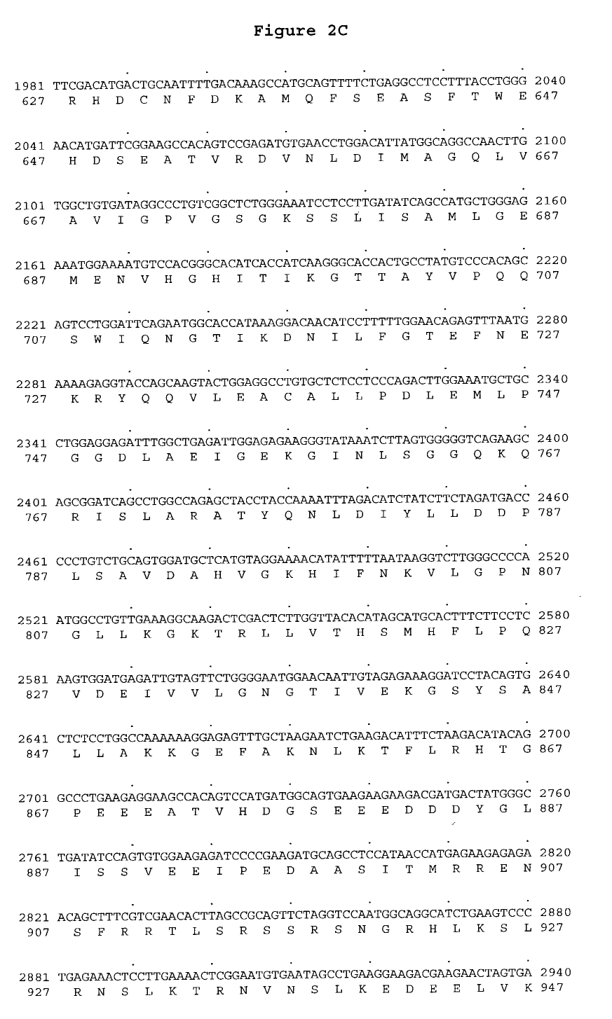

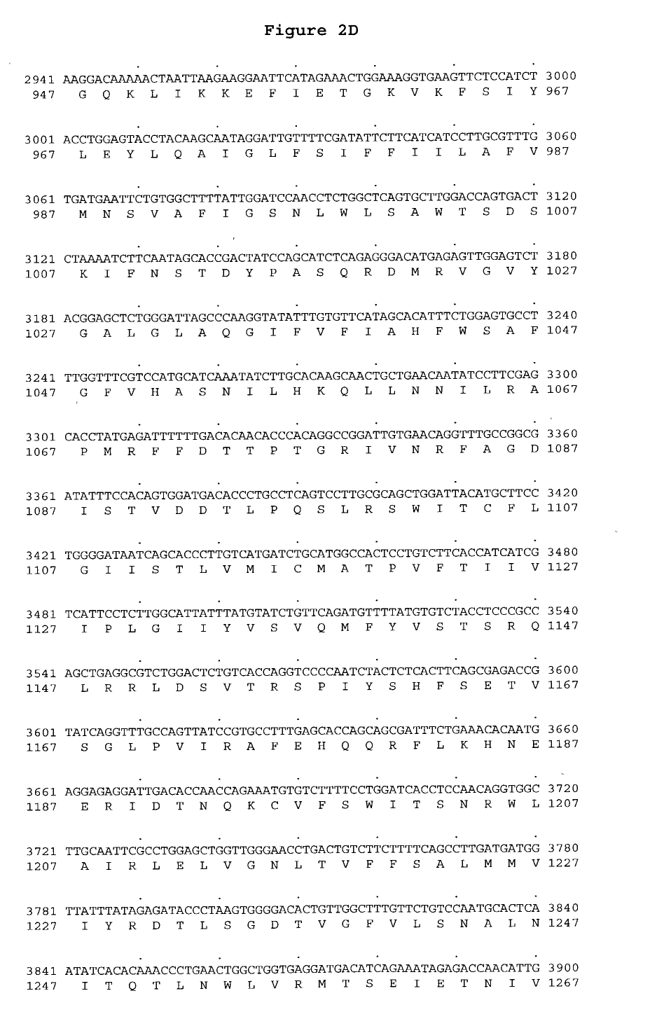

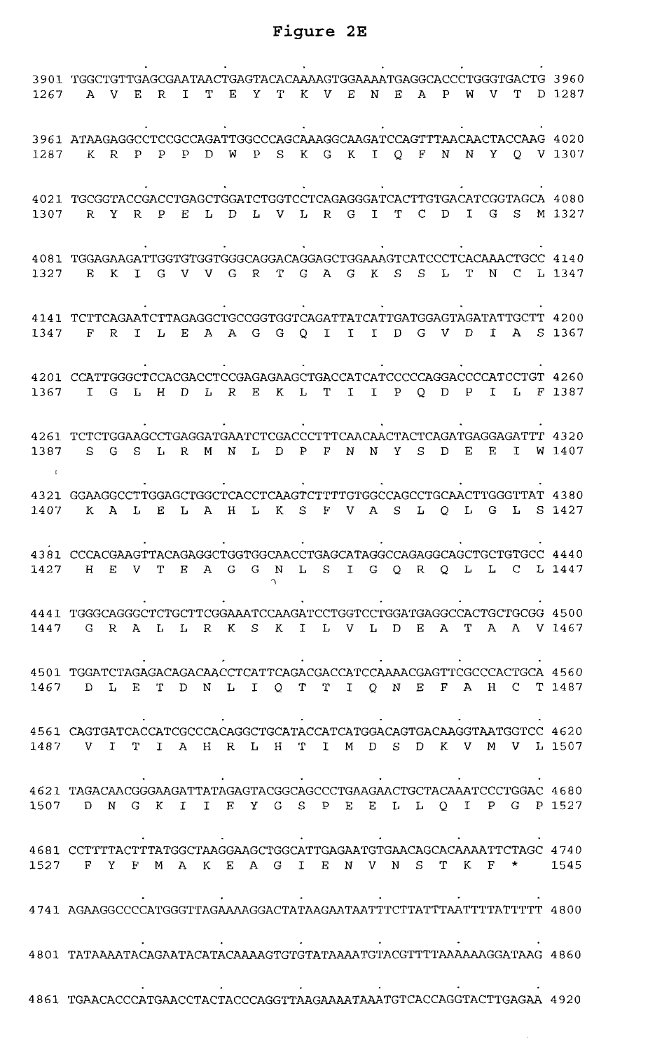

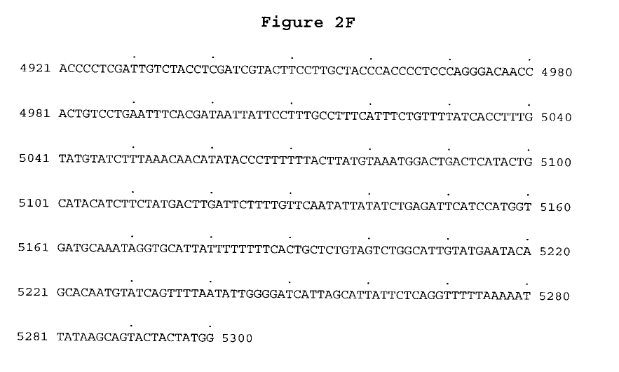

Table I provides a summary of the novel polypeptides and their encoding polynucleotides of the present invention.

Table II illustrates the preferred hybridization conditions for the polynucleotides of the present invention. Other hybridization conditions may be known in the art or described elsewhere herein.

Table III summarizes the single nucleotide polymorphisms (SNPs) of the present invention. ‘Gene Name’ refers to the gene in which the SNP resides; ° Coriell DNA Panel’ represents the number of DNA samples from the Coriell Institute, Collingswood, N.J. which were analyzed for each gene (see Table VII); ‘Total SNPs’ refers to the number of SNPs identified within each of the analyzed genes; ‘Misense’ and ‘Silent’ refer to the number of SNPs that either changed or did not change the amino acid sequence of the encoded polypeptide for each gene, respectfully; and ‘UTR’ and ‘Non-CDS’ refer to the number of SNPs which were found either within the “untranslated region” or “non-coding” region of the polynucleotide sequences of each gene, respectfully.

Table IV provides a detailed summary of the SNPs of the present invention (SEQ ID NO:51-119, and 120-188). ‘GENE_DESCRIPTION’ refers to the gene in which the SNP resides; ‘HGNC_ID’ refers to the gene symbol as designated by the HUGO Gene Nomenclature Committee; ‘SNP_ID’ refers to the unique name identifier associated with the SNP of the present invention; ‘CONTIG_NUM’ refers to the experimental sequence information of the contig in which the SNP was identified; ‘CONTIG_POS’ refers to the polynucleotide position within the experimental sequence contig at which the SNP resides; ‘FLANK_SEQ’ provides the genomic polynucleotide sequence of the gene immediately flanking the SNP—each sequence provides the reference (REF) and variable (ALT) nucleic acid residue at the polymorphic site according to the following format: 5′ Flanking polynucleotide sequence [REF/ALT] 3′ flanking polynucleotide sequence; ‘FLANK_SEQ REF (SEQ ID NO: )’ refers to the SEQ ID NO of the genomic polynucleotide sequence comprising the reference nucleic acid sequence within the Sequence Listing of the present invention; ‘FLANK_SEQ ALT (SEQ ID NO: )’ refers to the SEQ ID NO of the genomic polynucleotide sequence comprising the variable nucleic acid sequence within the Sequence Listing of the present invention; ‘REF_SEQ_ID’ refers to the Genbank Accession number of the reference genomic polynucleotide sequence in which the SNP resides, and which was used to design resequencing assays; ‘REF_SEQ_POS’ refers to the nucleotide position within the reference genomic polynucleotide sequence (REF_SEQ_ID) in which the polymorphism (SNP) resides; ‘REF_NT’ refers to the reference polymorphic nucleotide (SNP) allele within the reference genomic polynucleotide sequence; ‘ALT_NT’ refers to the variant polymorphic nucleotide (SNP) allele within the reference genomic polynucleotide sequence; ‘EXON’ refers to the location of the polymorphic nucleotide allele (SNP) within the gene structure of the referenced genomic polynucleotide sequence (putative exon/intron number) as determined using software programs well known in the art (e.g., BLAST2, Sim4, and/or GRAIL, etc.); ‘MUTATION_TYPE’ refers to the type of polymorphism according to the following classification: Missense—an SNP within the coding region of a gene resulting in a change in the encoded amino acid sequence, Silent—an SNP within the coding region of a gene but does not result in a change in the encoded amino acid sequence, and Non-CDS: an SNP that is located within the non-coding region (e.g., intron, untranslated region) of a gene; ‘REV_COMP’ refers to the relative 5′ to 3′ orientation of the reference genomic polynucleotide sequence compared to the cDNA polynucleotide sequence of the gene wherein ‘0’ indicates the genomic and cDNA sequences are in the same orientation, whereas ‘1’ indicates the genomic and cDNA sequences are in an opposing orientation; ‘REF_CODON’ refers to the reference nucleotide sequence of the codon in which the encoding SNPs reside; ‘ALT_CODON’ refers to the variable nucleotide sequence of the codon in which the encoding SNPs reside; ‘cDNA_SEQ_ID’ refers to the Genbank Accession Number for the cDNA gene sequence in which the SNP resides; and ‘cDNA_SEQ_POS’ refers to the nucleotide position of the SNP within the polynucleotide sequence of the cDNA.

Table V provides a detailed summary of the SNPs of the present invention comprising additional 5′ and 3′ flanking genomic sequence (SEQ ID NO:189-257, and 258-326). The Table headings are the same as in Table IV with the following exceptions: ‘REFSEQ_FLANK’ provides the genomic polynucleotide sequence of the gene flanking the SNP—each sequence provides the reference (REF) and variable (ALT) nucleic acid residue at the polymorphic site according to the following format: 5′ Flanking polynucleotide sequence [REF/ALT] 3′ flanking polynucleotide sequence; ‘REFSEQ_FLANK_ORIENT’ refers to the relative orientation (sense or antisense, 5′ to 3′ or 3′ to 5′) of the REFSEQ_FLANK polynucletide sequence with respect to the FLANK_SEQ polynucletide orientation wherein a “0” refers to the FLANK_SEQ and REFSEQ_FLANK polynucleotide sequences having the same orientation, as opposed to a “1” wherein the FLANK_SEQ and REFSEQ_FLANK polynucleotide sequences have an opposing orientation; ‘REFSEQ_FLANK REF (SEQ ID NO: )’ refers to the SEQ ID NO of the genomic polynucleotide sequence comprising the reference nucleic acid sequence within the Sequence Listing of the present invention; and ‘REFSEQ_FLANK ALT (SEQ ID NO: )’ refers to the SEQ ID NO of the genomic polynucleotide sequence comprising the variable nucleic acid sequence within the Sequence Listing of the present invention.

Table VI provides a detailed summary of the SNPs of the present invention which fall within the coding region of the captioned genes. The Table headings are the same as in Table IV and V with the following exceptions: ‘REF_AA’ refers to the reference amino acid within the reference protein sequence within which an encoding SNP of the present invention resides; ‘ALT_AA’ refers to the variant amino acid within the reference protein sequence affected by an encoding SNP of the present invention; ‘PROTEIN_ID’ refers to the Genbank Accession Number of the reference protein sequence; ‘PROTEIN_POS’ refers to the amino acid location affected by the encoding SNP within the reference protein sequence.

Table VII provides the ethnicity and sample ID for each of the Coriell DNA samples (Coriell Institute, Collingswood, N.J.) used in identifying the SNPs of the present invention. The table also identifies the plate number of the relevant samples used in the assays, as described elsewhere herein.

Table VIII provides a detailed summary of the various PCR primers that were used in amplifying relevant regions of the organic anion transport genes for single nucleotide polymorphism analysis. The Table headings are the same as in Table IV and V above with the following exceptions: ‘PCR Amplicon_Name’ refers to the name given to product of the PCR amplified DNA; ‘Target_Name’ refers to the name of the region of genomic DNA for each gene which was targeted for PCR amplification; ‘PCR Left primer’ refers to the 5′ primer used to amplify the target; ‘PCR Left primer (SEQ ID NO:)’ refers to the SEQ ID NO for this particular sequence within the Sequence Listing of the present invention; ‘PCR Right primer’ refers to the 3′ primer used to amplify the target; and ‘PCR Right primer (SEQ ID NO:)’ refers to the SEQ ID NO for this particular sequence within the Sequence Listing of the present invention.

Table IX provides a detailed summary of the various sequencing primers that were used in sequencing relevant regions of the organic anion transport genes (e.g., PCR Amplicons of Table VIII) for single nucleotide polymorphism analysis. The Table headings are the same as in Table IV, V, and VIII above with the following exceptions: ‘Forward Sequencing Primer’ refers to the 3′ (forward) primer used for sequencing across the PCR amplicon; ‘Forward_Seq_Name’ refers to the name given to the resulting forward sequence for a particular PCR amplicon; ‘Forward Sequencing Primer (SEQ ID NO:)’ refers to the SEQ ID NO for this particular sequence within the Sequence Listing of the present invention; ‘Reverse Sequencing Primer’ refers to the 5′ (reverse) primer used for sequencing across the PCR amplicon; ‘Reverse_Seq_Name’ refers to the name given to the resulting reverse sequence for a particular PCR amplicon; and ‘Reverse Sequencing Primer (SEQ ID NO:)’ refers to the SEQ ID NO for this particular sequence within the Sequence Listing of the present invention.

Table X provides a detailed summary of the various primers that were used in genotyping the single nucleotide polymorphisms of the organic anion transport gene SNPs of the present invention. The Table headings are the same as in Table IV, V, and VIII above with the following exceptions: ‘ORCHID_LEFT’ refers to the 3′ (forward) primer used for sequencing across the SNP loci of each respective SNP; ‘ORCHID_LEFT’ (SEQ ID NO:)’ refers to the SEQ ID NO for this particular sequence within the Sequence Listing of the present invention; ‘ORCHID_RIGHT’ refers to the 5′ (reverse) primer used for sequencing across the SNP loci of each respective SNP; ‘ORCHID_RIGHT’ (SEQ ID NO:)’ refers to the SEQ ID NO for this particular sequence within the Sequence Listing of the present invention; ‘ORCHID_SNPIT’ refers to the hybridization oligonucleotide used for single base extension; ‘ORCHID_SNPIT (SEQ ID NO:)’ refers to the SEQ ID NO for this particular sequence within the Sequence Listing of the present invention.

Table XI provides a detailed summary of the various primers that may be used in genotyping the single nucleotide polymorphisms of the organic anion transport gene SNPs of the present invention using the alternative GBS method described herein. The Table headings are the same as in Table IV, V, and VIII above with the following exceptions: ‘GBS_LEFT’ refers to the 3′ (forward) primer that may be used for sequencing across the SNP loci of each respective SNP; ‘GBS_LEFT (SEQ ID NO:)’ refers to the SEQ ID NO for this particular sequence within the Sequence Listing of the present invention; ‘GBS_RIGHT’ refers to the 5′ (reverse) primer that may be used for sequencing across the SNP loci of each respective SNP; and ‘GBS_RIGHT (SEQ ID NO:)’ refers to the SEQ ID NO for this particular sequence within the Sequence Listing of the present invention.

### DETAILED DESCRIPTION OF THE INVENTION

The present invention relates to a nucleic acid molecule which comprises a single nucleotide polymorphism (SNP) at a specific location. The nucleic acid molecule, e.g., a gene, which includes the SNP has at least two alleles, referred to herein as the reference allele and the variant allele. The reference allele (prototypical or wild type allele) has been designated arbitrarily and typically corresponds to the nucleotide sequence of the native form of the nucleic acid molecule. The variant allele differs from the reference allele by one nucleotide at the site(s) identified in the Table IV, V, and/or VI. The present invention also relates to variant alleles of the described genes and to complements of the variant alleles. The invention further relates to portions of the variant alleles and portions of complements of the variant alleles which comprise (encompass) the site of the SNP and are at least nucleotides in length. Portions can be, for example, 5-10,5-15, 10-20,5-25, 10-30, 10- or 10-100 bases long. F or example, a portion of a variant allele which is nucleotides in length includes the single nucleotide polymorphism (the nucleotide which differs from the reference allele at that site) and twenty additional nucleotides which flank the site in the variant allele. These additional nucleotides can be on one or both sides of the polymorphism. Polymorphisms which are the subject of this invention are defined in Table IV, V, or VI herein.

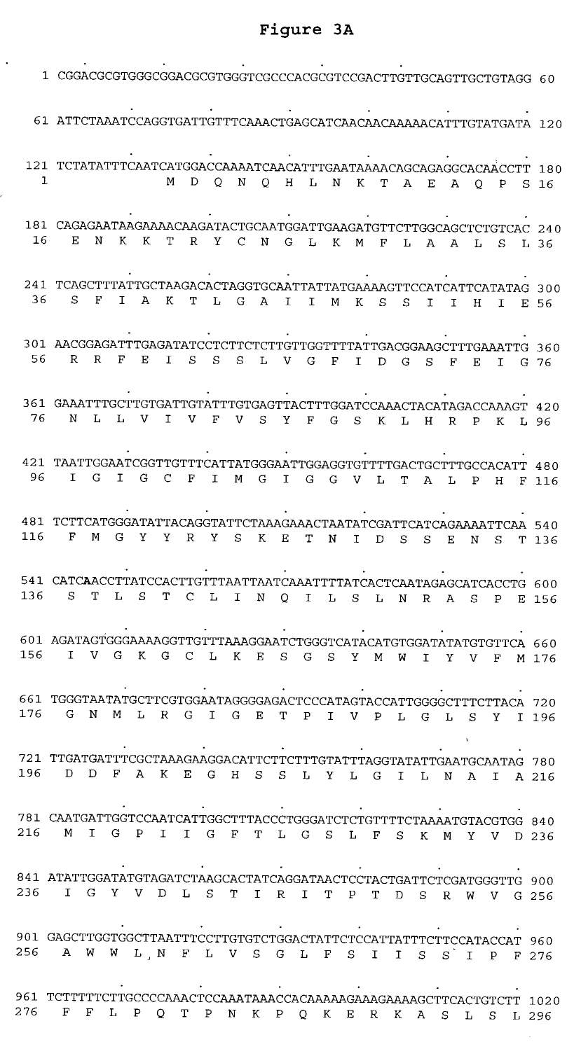

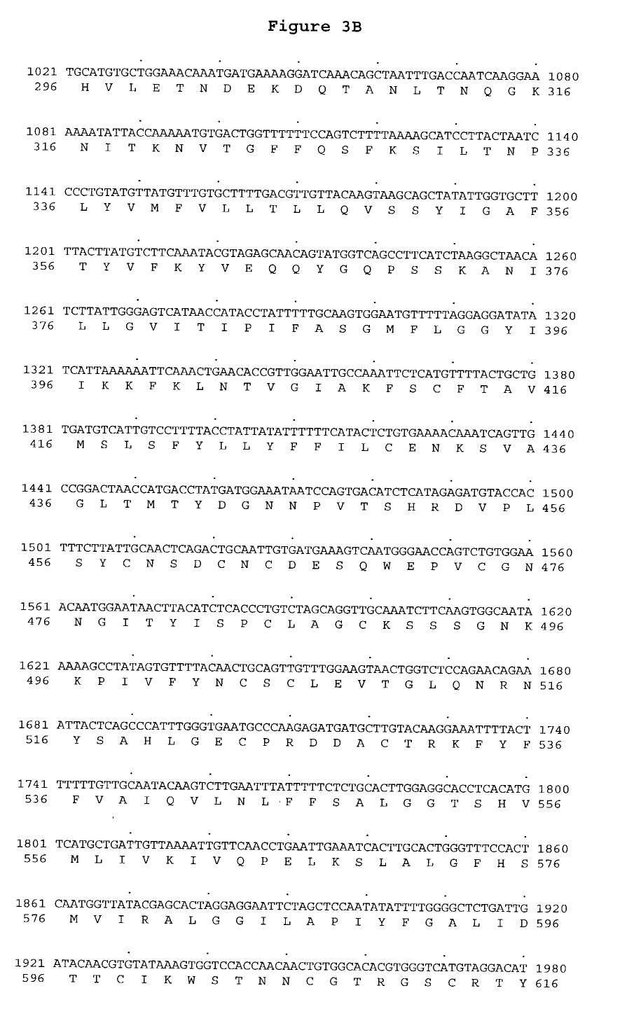

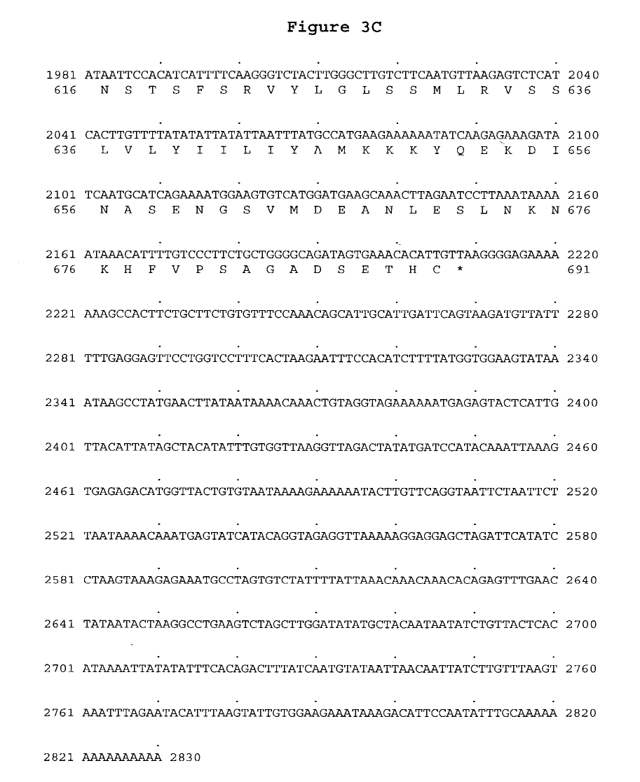

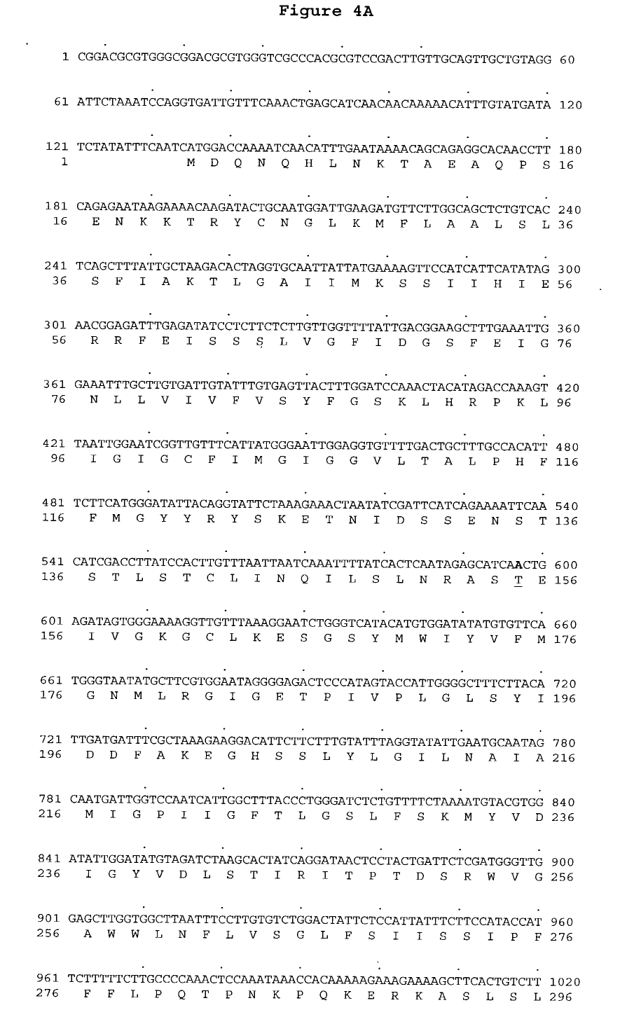

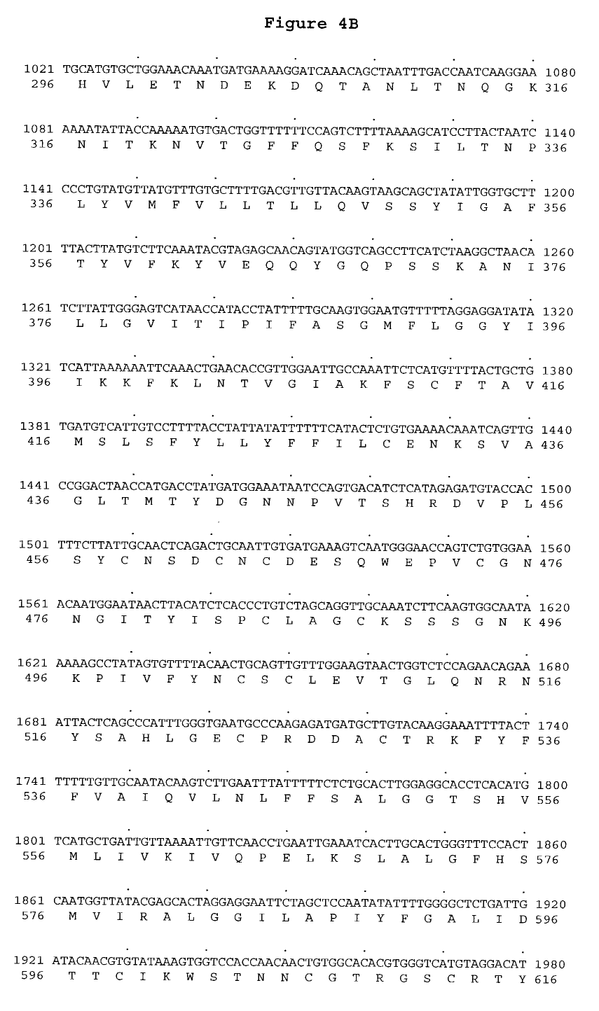

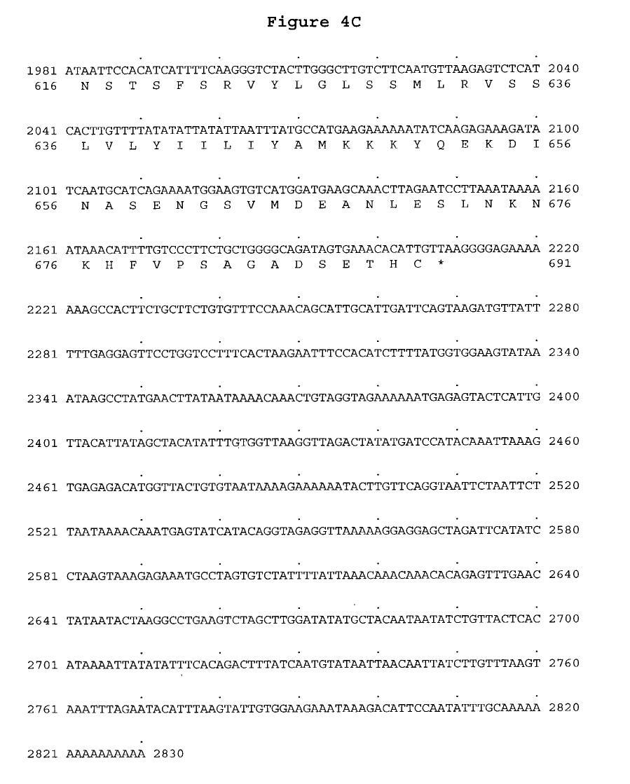

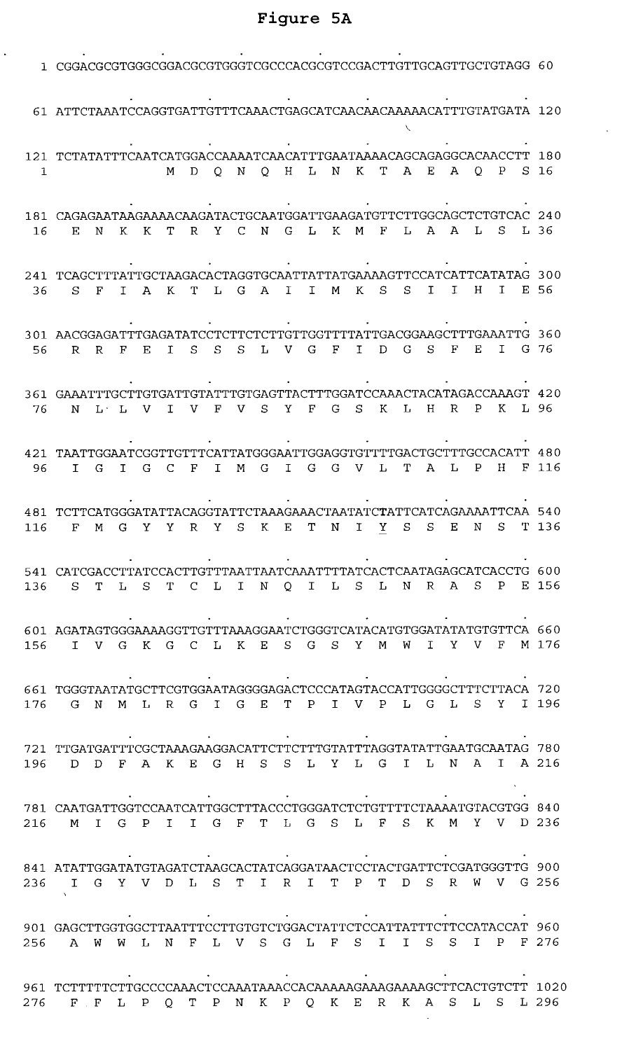

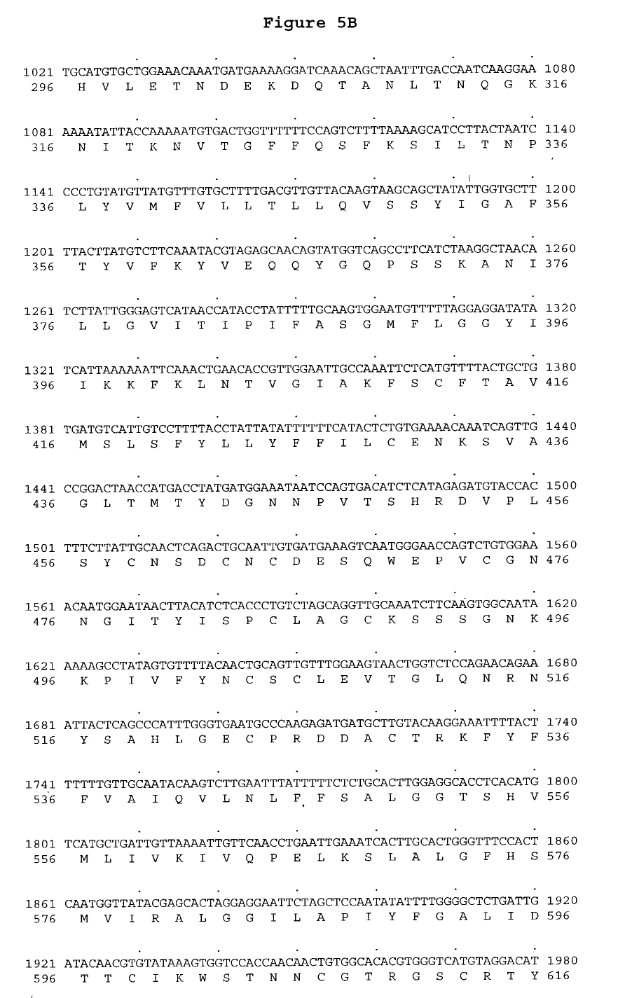

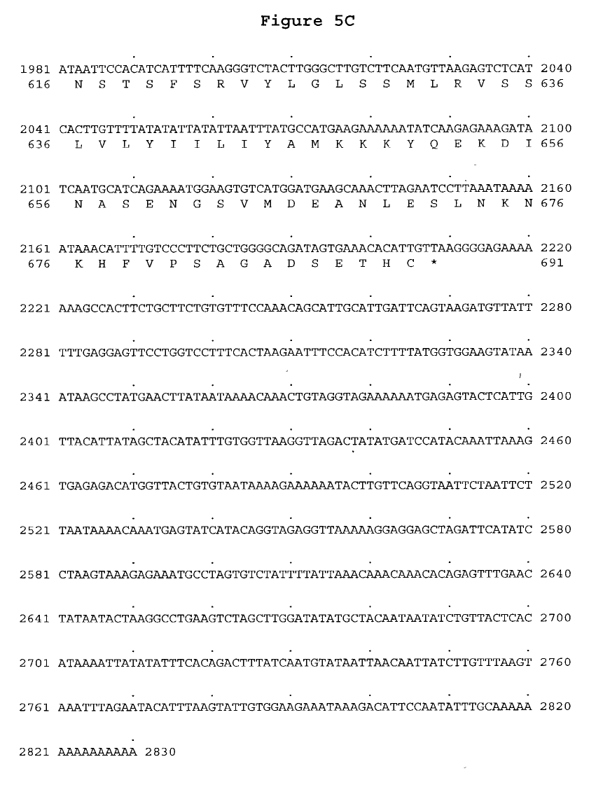

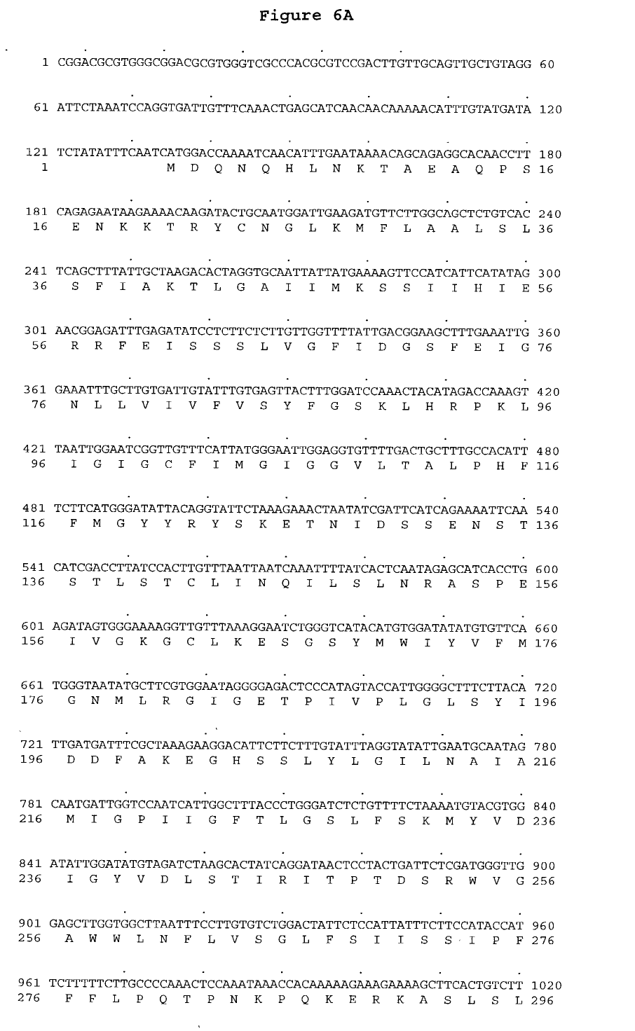

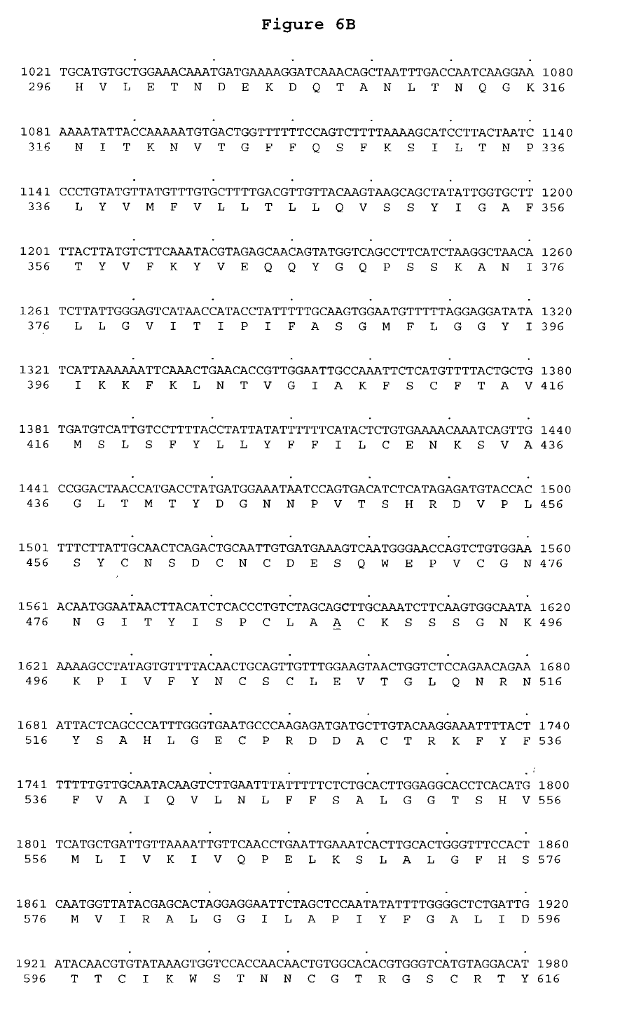

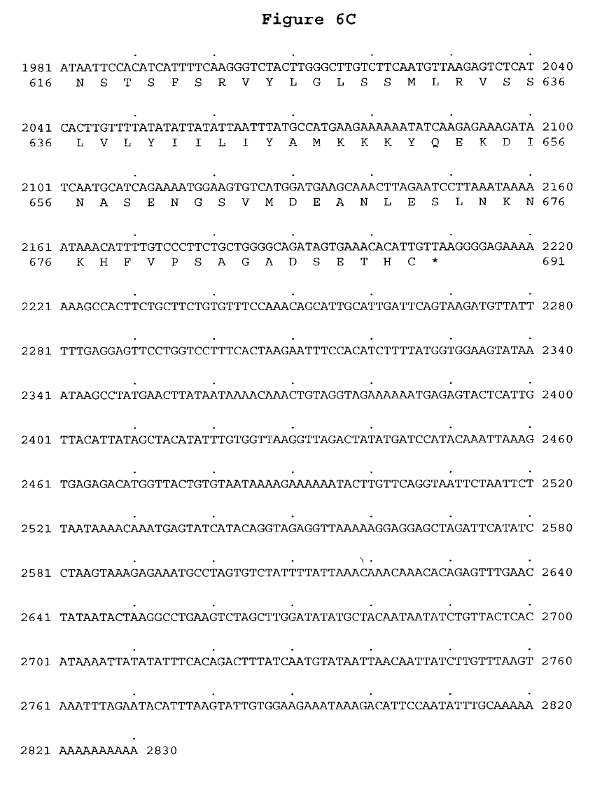

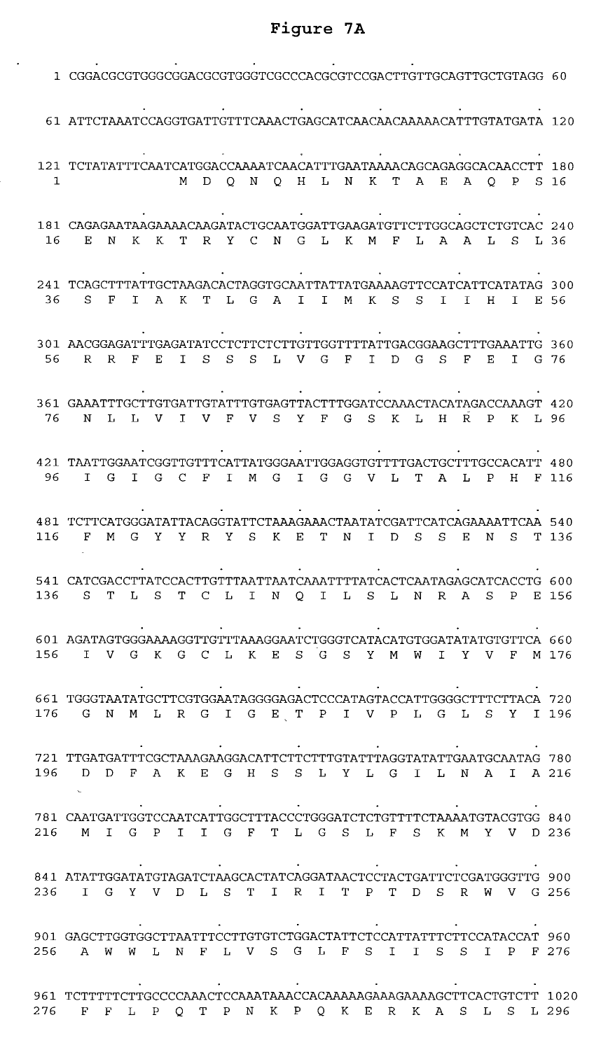

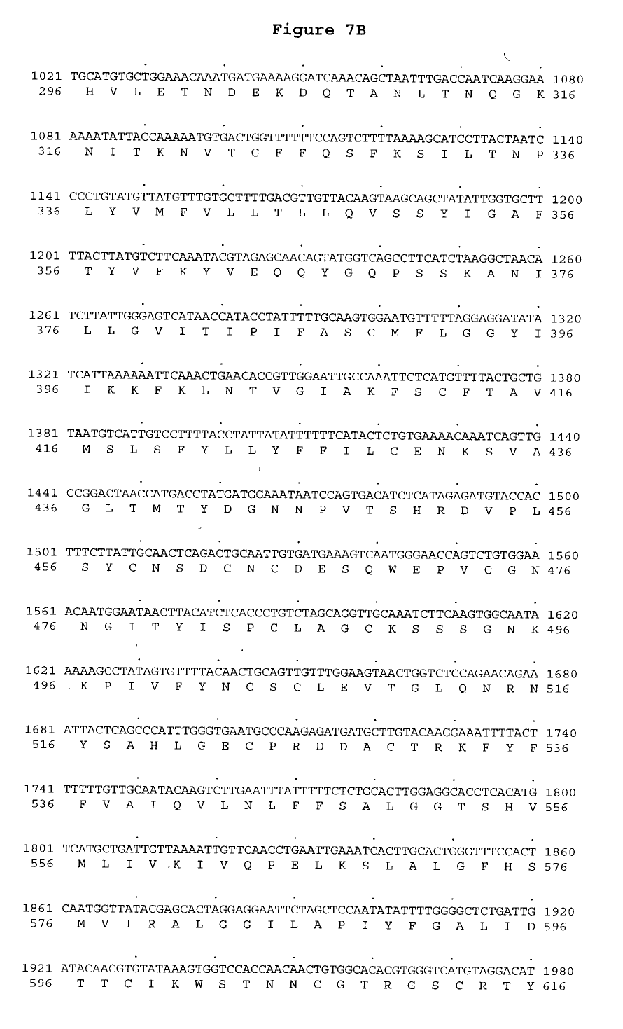

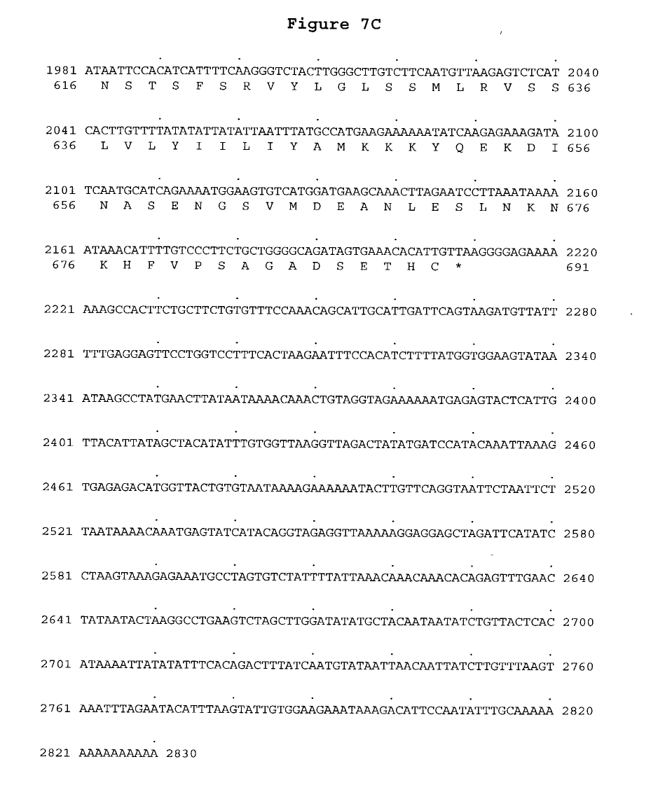

For example, the invention relates to a portion of a gene (e.g., OATP2, solute carrier family 21 member 6 (SLC21A6)) having a nucleotide sequence according to FIGS. 3A-C (SEQ ID NO:5) comprising a single nucleotide polymorphism at a specific position (e.g., nucleotide 545). The reference nucleotide for this polymorphic form of OATP2 is shown in the ‘FLANK_SEQ (REF/ALT)’ column as the “REF” nucleotide (in this case, the “REF” nucletide is “G”) of Table IV, and the variant nucleotide is shown in the ‘FLANK_SEQ (REF/ALT)’ column as the “ALT” nucleotide of Table IV (in this case, the “ALT” nucleotide is an “A”). In a preferred embodiment, the nucleic acid molecule of the invention comprises the variant (alternate) nucleotide at the polymorphic position. For example, the invention relates to a nucleic acid molecule which comprises the nucleic acid sequence shown in the ‘FLANK_SEQ (REF/ALT)’ as the “ALT” nucleotide in Table IV having an “A” at nucleotide position 545 of FIGS. 3A-C (SEQ ID NO:5). The nucleotide sequences of the invention can be double- or single-stranded.

The invention further relates to other portions of a gene as described herein containing a polymorphic locus, preferably comprising the polymorphic allele (i.e., the ‘ALT’ allele, or variant allele).

The invention further provides allele-specific oligonucleotides that hybridize to a gene comprising a single nucleotide polymorphism or to the complement of the gene. Such oligonucleotides will hybridize to one polymorphic form of the nucleic acid molecules described herein but not to the other polymorphic form(s) of the sequence. Thus, such oligonucleotides can be used to determine the presence or absence of particular alleles of the polymorphic sequences described herein. These oligonucleotides can be probes or primers.

The invention further provides a method of analyzing a nucleic acid from an individual. The method determines which base is present at any one of the polymorphic sites shown in Tables I, IV, V, or VI. Optionally, a set of bases occupying a set of the polymorphic sites shown in Tables I, IV, V, or VI is determined. This type of analysis can be performed on a number of individuals, who are also tested (previously, concurrently or subsequently) for the presence of a disease phenotype. The presence or absence of disease phenotype is then correlated with a base or set of bases present at the polymorphic site or sites in the individuals tested.

Thus, the invention further relates to a method of predicting the presence, absence, likelihood of the presence or absence, or severity of a particular phenotype or disorder associated with a particular genotype. The method comprises obtaining a nucleic acid sample from an individual and determining the identity of one or more bases (nucleotides) at polymorphic sites of nucleic acid molecules described herein, wherein the presence of a particular base is correlated with a specified phenotype or disorder, thereby predicting the presence, absence, likelihood of the presence or absence, or severity of the phenotype or disorder in the individual. The correlation between a particular polymorphic form of agene and a phenotype can thus be used in methods of diagnosis of that phenotype, as well as in the development of treatments for the phenotype.

The invention further relates to a polynucleotide encoding a polypeptide fragment of SEQ ID NO:6, 8, 10, 12, 14, 16, 18, 20, 22, 24, 26, 28, 30, 32, 34, 36, 38, 40, 42, 44, 46, 48, 50, and/or 604, or a polypeptide fragment encoded by the cDNA sequence included in the deposited clone, which is hybridizable to SEQ ID NO:5, 7, 9, 11, 13, 15, 17, 19, 21, 23, 25, 27, 29, 31, 33, 35, 37, 39, 41, 43, 45, 47, 49, and/or 603.

The invention further relates to a polynucleotide encoding a polypeptide domain of SEQ ID NO:6, 8, 10, 12, 14, 16, 18, 20, 22, 24, 26, 28, 30, 32, 34, 36, 38, 40, 42, 44, 46, 48, 50, and/or 604 or a polypeptide domain encoded by the cDNA sequence included in the deposited clone, which is hybridizable to SEQ ID NO:5, 7, 9, 11, 13, 15, 17, 19, 21, 23, 25, 27, 29, 31, 33, 35, 37, 39, 41, 43, 45, 47, 49, and/or 603.

The invention further relates to a polynucleotide encoding a polypeptide epitope of SEQ ID NO:6, 8, 10, 12, 14, 16, 18, 20, 22, 24, 26, 28, 30, 32, 34, 36, 38, 40, 42, 44, 46, 48, 50, and/or 604 or a polypeptide epitope encoded by the cDNA sequence included in the deposited clone, which is hybridizable to SEQ ID NO:5, 7, 9, 11, 13, 15, 17, 19, 21, 23, 25, 27, 29, 31, 33, 35, 37, 39, 41, 43, 45, 47, 49, and/or 603.

The invention further relates to a polynucleotide encoding a polypeptide of SEQ ID NO:6, 8, 10, 12, 14, 16, 18, 20, 22, 24, 26, 28, 30, 32, 34, 36, 38, 40, 42, 44, 46, 48, 50, and/or 604 or the cDNA sequence included in the deposited clone, which is hybridizable to SEQ ID NO:5, 7, 9, 11, 13, 15, 17, 19, 21, 23, 25, 27, 29, 31, 33, 35, 37, 39, 41, 43, 45, 47, 49, and/or 603, having biological activity.

The invention further relates to a polynucleotide which is a variant of SEQ ID NO:5, 7, 9, 11, 13, 15, 17, 19, 21, 23, 25, 27, 29, 31, 33, 35, 37, 39, 41, 43, 45, 47, 49, and/or 603.

The invention further relates to a polynucleotide which is an allelic variant of SEQ ID NO:5, 7, 9, 11, 13, 15, 17, 19, 21, 23, 25, 27, 29, 31, 33, 35, 37, 39, 41, 43, 45, 47, 49, and/or 603.

The invention further relates to a polynucleotide which encodes a species homologue of the SEQ ID NO:6, 8, 10, 12, 14, 16, 18, 20, 22, 24, 26, 28, 30, 32, 34, 36, 38, 40, 42, 44, 46, 48, 50, and/or 604.

The invention further relates to a polynucleotide which represents the complimentary sequence (antisense) of SEQ ID NO:5, 7, 9, 11, 13, 15, 17, 19, 21, 23, 25, 27, 29, 31, 33, 35, 37, 39, 41, 43, 45, 47, 49, and/or 603.

The invention further relates to a polynucleotide capable of hybridizing under stringent conditions to any one of the polynucleotides specified herein, wherein said polynucleotide does not hybridize under stringent conditions to a nucleic acid molecule having a nucleotide sequence of only A residues or of only T residues.

The invention further relates to an isolated nucleic acid molecule of SEQ ID NO:6, 8, 10, 12, 14, 16, 18, 20, 22, 24, 26, 28, 30, 32, 34, 36, 38, 40, 42, 44, 46, 48, 50, and/or 604, wherein the polynucleotide fragment comprises a nucleotide sequence encoding an organic anion transport protein.

The invention further relates to an isolated nucleic acid molecule of SEQ ID NO:5, 7, 9, 11, 13, 15, 17, 19, 21, 23, 25, 27, 29, 31, 33, 35, 37, 39, 41, 43, 45, 47, 49, and/or 603, wherein the polynucleotide fragment comprises a nucleotide sequence encoding the sequence identified as SEQ ID NO:6, 8, 10, 12, 14, 16, 18, 20, 22, 24, 26, 28, 30, 32, 34, 36, 38, 40, 42, 44, 46, 48, 50, and/or 604 or the polypeptide encoded by the cDNA sequence included in the deposited clone, which is hybridizable to SEQ ID NO:5, 7, 9, 11, 13, 15, 17, 19, 21, 23, 25, 27, 29, 31, 33, 35, 37, 39, 41, 43, 45, 47, 49, and/or 603.

The invention further relates to an isolated nucleic acid molecule of of SEQ ID NO:5, 7, 9, 11, 13, 15, 17, 19, 21, 23, 25, 27, 29, 31, 33, 35, 37, 39, 41, 43, 45, 47, 49, and/or 603, wherein the polynucleotide fragment comprises the entire nucleotide sequence of SEQ ID NO:5, 7, 9, 11, 13, 15, 17, 19, 21, 23, 25, 27, 29, 31, 33, 35, 37, 39, 41, 43, 45, 47, 49, and/or 603 or the cDNA sequence included in the deposited clone, which is hybridizable to SEQ ID NO:5, 7, 9, 11, 13, 15, 17, 19, 21, 23, 25, 27, 29, 31, 33, 35, 37, 39, 41, 43, 45, 47, 49, and/or 603.

The invention further relates to an isolated nucleic acid molecule of SEQ ID NO:5, 7, 9, 11, 13, 15, 17, 19, 21, 23, 25, 27, 29, 31, 33, 35, 37, 39, 41, 43, 45, 47, 49, and/or 603, wherein the nucleotide sequence comprises sequential nucleotide deletions from either the C-terminus or the N-terminus.

The invention further relates to an isolated polypeptide comprising an amino acid sequence that comprises a polypeptide fragment of SEQ ID NO:6, 8, 10, 12, 14, 16, 18, 20, 22, 24, 26, 28, 30, 32, 34, 36, 38, 40, 42, 44, 46, 48, 50, and/or 604 or the encoded sequence included in the deposited clone.

The invention further relates to a polypeptide fragment of SEQ ID NO:6, 8, 10, 12, 14, 16, 18, 20, 22, 24, 26, 28, 30, 32, 34, 36, 38, 40, 42, 44, 46, 48, 50, and/or 604 or the encoded sequence included in the deposited clone, having biological activity.

The invention further relates to a polypeptide domain of SEQ ID NO:6, 8, 10, 12, 14, 16, 18, 20, 22, 24, 26, 28, 30, 32, 34, 36, 38, 40, 42, 44, 46, 48, 50, and/or 604 or the encoded sequence included in the deposited clone.

The invention further relates to a polypeptide epitope of SEQ ID NO:6, 8, 10, 12, 14, 16, 18, 20, 22, 24, 26, 28, 30, 32, 34, 36, 38, 40, 42, 44, 46, 48, 50, and/or 604 or the encoded sequence included in the deposited clone.

The invention further relates to a full length protein of SEQ ID NO:6, 8, 10, 12, 14, 16, 18, 20, 22, 24, 26, 28, 30, 32, 34, 36, 38, 40, 42, 44, 46, 48, 50, and/or 604 or the encoded sequence included in the deposited clone.

The invention further relates to a variant of SEQ ID NO:6, 8, 10, 12, 14, 16, 18, 20, 22, 24, 26, 28, 30, 32, 34, 36, 38, 40, 42, 44, 46, 48, 50, and/or 604.

The invention further relates to an allelic variant of SEQ ID NO:6, 8, 10, 12, 14, 16, 18, 20, 22, 24, 26, 28, 30, 32, 34, 36, 38, 40, 42, 44, 46, 48, 50, and/or 604.

The invention further relates to a species homologue of SEQ ID NO:6, 8, 10, 12, 14, 16, 18, 20, 22, 24, 26, 28, 30, 32, 34, 36, 38, 40, 42, 44, 46, 48, 50, and/or 604.

The invention further relates to the isolated polypeptide of of SEQ ID NO:6, 8, 10, 12, 14, 16, 18, 20, 22, 24, 26, 28, 30, 32, 34, 36, 38, 40, 42, 44, 46, 48, 50, and/or 604, wherein the full length protein comprises sequential amino acid deletions from either the C-terminus or the N-terminus.

The invention further relates to an isolated antibody that binds specifically to the isolated polypeptide of SEQ ID NO:6, 8, 10, 12, 14, 16, 18, 20, 22, 24, 26, 28, 30, 32, 34, 36, 38, 40, 42, 44, 46, 48, 50, and/or 604.

The invention further relates to a method for preventing, treating, or ameliorating a medical condition, comprising administering to a mammalian subject a therapeutically effective amount of the polypeptide of SEQ ID NO:6, 8, 10, 12, 14, 16, 18, 20, 22, 24, 26, 28, 30, 32, 34, 36, 38, 40, 42, 44, 46, 48, 50, and/or 604 or the polynucleotide of SEQ ID NO:5, 7, 9,11, 13, 15, 17, 19, 21, 23, 25, 27, 29, 31, 33, 35, 37, 39, 41, 43, 45, 47, 49, and/or 603.

The invention further relates to a method of diagnosing a pathological condition or a susceptibility to a pathological condition in a subject comprising the steps of (a) determining the presence or absence of a mutation in the polynucleotide of SEQ ID NO:5, 7, 9, 11, 13, 15, 17, 19, 21, 23, 25, 27, 29, 31, 33, 35, 37, 39, 41, 43, 45, 47, 49, and/or 603; and (b) diagnosing a pathological condition or a susceptibility to a pathological condition based on the presence or absence of said mutation.

The invention further relates to a method of diagnosing a pathological condition or a susceptibility to a pathological condition in a subject comprising the steps of (a) determining the presence or amount of expression of the polypeptide of of SEQ ID NO:6, 8, 10, 12, 14, 16, 18, 20, 22, 24, 26, 28, 30, 32, 34, 36, 38, 40, 42, 44, 46, 48, 50, and/or 604 in a biological sample; and diagnosing a pathological condition or a susceptibility to a pathological condition based on the presence or amount of expression of the polypeptide.

The invention further relates to a method for identifying a binding partner to the polypeptide of SEQ ID NO:6, 8, 10, 12, 14, 16, 18, 20, 22, 24, 26, 28, 30, 32, 34, 36, 38, 40, 42, 44, 46, 48, 50, and/or 604 comprising the steps of (a) contacting the polypeptide of SEQ ID NO:6, 8, 10, 12, 14, 16, 18, 20, 22, 24, 26, 28, 30, 32, 34, 36, 38, 40, 42, 44, 46, 48, 50, and/or 604 with a binding partner; and (b) determining whether the binding partner effects an activity of the polypeptide.

The invention further relates to a gene corresponding to the cDNA sequence of SEQ ID NO:5, 7, 9, 11, 13, 15, 17, 19, 21, 23, 25, 27, 29, 31, 33, 35, 37, 39, 41, 43, 45, 47, 49, and/or 603.

The invention further relates to a method of identifying an activity in a biological assay, wherein the method comprises the steps of expressing SEQ ID NO:5, 7, 9, 11, 13, 15, 17, 19, 21, 23, 25, 27, 29, 31, 33, 35, 37, 39, 41, 43, 45, 47, 49, and/or 603 in a cell, (b) isolating the supernatant; (c) detecting an activity in a biological assay; and (d) identifying the protein in the supernatant having the activity.

The invention further relates to a process for making polynucleotide sequences encoding gene products having altered activity selected from the group consisting of SEQ ID NO:6, 8, 10, 12, 14, 16, 18, 20, 22, 24, 26, 28, 30, 32, 34, 36, 38, 40, 42, 44, 46, 48, 50, and/or 604 activity comprising the steps of (a) shuffling a nucleotide sequence of SEQ ID NO:5, 7, 9, 11, 13, 15, 17, 19, 21, 23, 25, 27, 29, 31, 33, 35, 37, 39, 41, 43, 45, 47, 49, and/or 603, (b) expressing the resulting shuffled nucleotide sequences and, (c) selecting for altered activity selected from the group consisting of SEQ ID NO:6, 8, 10, 12, 14, 16, 18, 20, 22, 24, 26, 28, 30, 32, 34, 36, 38, 40, 42, 44, 46, 48, 50, and/or 604 activity as compared to the activity selected from the group consisting of SEQ ID NO:6, 8, 10, 12, 14, 16, 18, 20, 22, 24, 26, 28, 30, 32, 34, 36, 38, 40, 42, 44, 46, 48, 50, and/or 604 activity of the gene product of said unmodified nucleotide sequence.

The invention further relates to a shuffled polynucleotide sequence produced by a shuffling process, wherein said shuffled DNA molecule encodes a gene product having enhanced tolerance to an inhibitor of any one of the activities selected from the group consisting of SEQ ID NO:6, 8, 10, 12, 14, 16, 18, 20, 22, 24, 26, 28, 30, 32, 34, 36, 38, 40, 42, 44, 46, 48, 50, and/or 604 activity.

The invention further relates to a method for preventing, treating, or ameliorating a medical condition with the polypeptide provided as SEQ ID NO:6, 8, 10, 12, 14, 16, 18, 20, 22, 24, 26, 28, 30, 32, 34, 36, 38, 40, 42, 44, 46, 48, 50, and/or 604, in addition to, its encoding nucleic acid, wherein the medical condition is a hepatic disorder

The invention further relates to a method for preventing, treating, or ameliorating a medical condition with the polypeptide provided as SEQ ID NO:6, 8, 10, 12, 14, 16, 18, 20, 22, 24, 26, 28, 30, 32, 34, 36, 38, 40, 42, 44, 46, 48, 50, and/or 604, in addition to, its encoding nucleic acid, wherein the medical condition is a metablic disorder.

The invention further relates to a method for preventing, treating, or ameliorating a medical condition with the polypeptide provided as SEQ ID NO:6, 8, 10, 12, 14, 16, 18, 20, 22, 24, 26, 28, 30, 32, 34, 36, 38, 40, 42, 44, 46, 48, 50, and/or 604, in addition to, its encoding nucleic acid, wherein the medical condition is a disorder that is treatable with pravastatin.

The invention further relates to a method for preventing, treating, or ameliorating a medical condition with the polypeptide provided as SEQ ID NO:6, 8, 10, 12, 14, 16, 18, 20, 22, 24, 26, 28, 30, 32, 34, 36, 38, 40, 42, 44, 46, 48, 50, and/or 604, in addition to, its encoding nucleic acid, wherein the medical condition is a disorder that is treatable with any of the known statins.

The invention further relates to a method for preventing, treating, or ameliorating a medical condition with the polypeptide provided as SEQ ID NO:6, 8, 10, 12, 14, 16, 18, 20, 22, 24, 26, 28, 30, 32, 34, 36, 38, 40, 42, 44, 46, 48, 50, and/or 604, in addition to, its encoding nucleic acid, wherein the medical condition is a hormonal disorder.

The invention relates to a nucleic acid molecule comprising a nucleic acid sequence selected from the group consisting of the nucleic acid sequences listed in Table I, Table IV, and Table V, wherein said nucleic acid sequence is at least 15 nucleotides in length and comprises a polymorphic site identified in Table I, IV, and V and wherein the nucleotide at the polymorphic site is different from a nucleotide at the polymorphic site in a corresponding reference allele.

The invention relates to a nucleic acid molecule with a nucleic acid sequence that is at least 20, 30, or 40 nucleotides in length.

The invention relates to a nucleic acid molecule wherein the nucleotide at the polymorphic site is the variant nucleotide for the nucleic acid sequence.

The invention relates to a nucleic acid molecule according wherein the nucleotide at the polymorphic site is the reference nucleotide for the nucleic acid sequence.

The invention further relates to an allele-specific oligonucleotide that hybridizes to a portion of a nucleic acid sequence selected from the group consisting of the nucleic acid sequences listed in Table I, Table IV, and Table V, wherein said portion is at least 15 nucleotides in length and comprises a polymorphic site identified in Table I, IV, or Table V, and wherein the nucleotide at the polymorphic site is different from a nucleotide at the polymorphic site in a corresponding reference allele.

The invention relates to an allele-specific oligonucleotide that is a probe and/or primer.

The invention relates to an allele-specific oligonucleotide wherein a central position of the probe aligns with the polymorphic site of the portion.

The invention further relates to an allele-specific oligonucleotide wherein the 3′ end of the primer aligns with the polymorphic site of the portion.

The invention relates to a method of analyzing a nucleic acid sample, comprising obtaining the nucleic acid sample from an individual; and determining a base occupying any one of the polymorphic sites shown in Table I, IV, V, or VI.

The invention relates to a method wherein the nucleic acid sample is obtained from a plurality of individuals, and a base occupying one of the polymorphic positions is determined in each of the individuals, and wherein the method further comprises testing each individual for the presence of a disease phenotype, and correlating the presence of the disease phenotype with the base.

The invention relates to a method of constructing haplotypes using the isolated nucleic acids provided herein comprising the step of grouping said nucleic acids. The invention also encompasses such a method further comprising the step of using said haplotypes to identify an individual for the presence of a disease phenotype, and correlating the presence of the disease phenotype with said haplotype. The invention relates to a nucleic acid molecule which represents the complementary sequence of the nucleic acid molecules provided herein. The invention relates to such a method further comprising the step of quantifying the nucleic acid sample comprising the polymorphic base.

### Definitions

An oligonucleotide can be DNA or RNA, and single- or double-stranded. Oligonucleotides can be naturally occurring or synthetic, but are typically prepared by synthetic means. Preferred oligonucleotides of the invention include segments of DNA, or their complements, which include any one of the polymorphic sites shown or described in Tables I, IV, V, or VI. The segments can be between 20 and 250 bases, and, in specific embodiments, are between 5-10, 5-20, 10-20, 10-50, 20-50 or 10-100 bases. For example, the segment can be about 20 bases. The polymorphic site can occur within any position of the segment. The segments can be from any of the allelic forms of DNA shown or described in Tables I, IV, V, or VI.

As used herein, the terms “nucleotide”, “base” and “nucleic acid” are intended to be equivalent. The terms “nucleotide sequence”, “nucleic acid sequence”, “nucleic acid molecule” and “segment” are intended to be equivalent.

Hybridization probes are oligonucleotides which bind in a base-specific manner to a complementary strand of nucleic acid. Such probes include peptide nucleic acids, as described in Nielsen et al., Science 254, 1497-1500 (1991). Probes can be any length suitable for specific hybridization to the target nucleic acid sequence. The most appropriate length of the probe may vary depending upon the hybridization method in which it is being used; for example, particular lengths may be more appropriate for use in microfabricated arrays, while other lengths may be more suitable for use in classical hybridization methods. Such optimizations are known to the skilled artisan. Suitable probes and primers can range from about nucleotidesto about nucleotides in length. For example, probes and primers can be 5, 6, 8, 10, 12, 14, 16, 18, 20, 22, 24, 25, 26, or 40 nucleotides in length. The probe or primer preferably overlaps at least one polymorphic site occupied by any of the possible variant nucleotides. The nucleotide sequence can correspond to the coding seqllence of the allele or to the complement of the coding sequence of the allele.

As used herein, the term “primer” refers to a single-stranded oligonucleotide which acts as a point of initiation of template-directed DNA synthesis under appropriate conditions (e.g., in the presence of four different nucleoside triphosphates and an agent for polymerization, such as DNA or RNA polymerase or reverse transcriptase) in an appropriate buffer and at a suitable temperature. The appropriate length of a primer depends on the intended use of the primer, but typically ranges from to nucleotides. Short primer molecules generally require cooler temperatures to form sufficiently stable hybrid complexes with the template. A primer need not reflect the exact sequence of the template, but must be sufficiently complementary to hybridize with a template. The term primer site refers to the area of the target DNA to which a primer hybridizes. The term primer pair refers to a set of primers including a 5′ (upstream) primer that hybridizes with the 5′ end of the DNA sequence to be amplified and a 3′ (downstream) primer that hybridizes with the complement of the 3′ end of the sequence to be amplified.

As used herein, linkage describes the tendency of genes, alleles, loci or genetic markers to be inherited together as a result of their location on the same chromosome. It can be measured by percent recombination between the two genes, alleles, loci or genetic markers.

As used herein, polymorphism refers to the occurance of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wild type form. Diploid organisms may be homozygous or heterozygous for allelic forms. A diallelic or biallelic polymorphism has two forms. A triallelic polymorphism has three forms.

Work described herein pertains to the resequencing of large numbers of genes in a large number of individuals to identify polymorphisms which may predispose individuals to disease. For example, polymorphisms in genes which are expressed in liver may predispose individuals to disorders of the liver. Likewise, polymorphisms in genes which are expressed in cardiovascular tissue may predispose individuals to disorders of the heart and/or circulatory system.

By altering amino acid sequence, SNPs may alter the function of the encoded proteins. The discovery of the SNP facilitates biochemical analysis of the variants and the development of assays to characterize the variants and to screen for pharmaceutical that would interact directly with on or another form of the protein. SNPs (including silent SNPs) may also alter the regulation of the gene at the transcriptional or post-transcriptional level. SNPs (including silent SNPs) also enable the development of specific DNA, RNA, or protein-based diagnostics that detect the presence or absence of the polymorphism in particular conditions.

A single nucleotide polymorphism occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations).

A single nucleotide polymorphism usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele. Typically the polymorphic site is occupied by a base other than the reference base. For example, where the reference allele contains the base “T” at the polymorphic site, the altered allele can contain a “C”, “G” or “A” at the polymorphic site.

For the purposes of the present invention the terms “polymorphic position”, “polymorphic site”, “polymorphic locus”, and “polymorphic allele” shall be construed to be equivalent and are defined as the location of a sequence identified as having more than one nucleotide represented at that location in a population comprising at least one or more individuals, and/or chromosomes.

Hybridizations are usually performed under stringent conditions, for example, at a salt concentration of no more than 1 M and a temperature of at least 25° C. For example, conditions of 5×SSPE (750 mM NaCl, mM NaPhosphate, mM EDT A, pH 7.4) and a temperature of 25-30° C., or equivalent conditions, are suitable for allele-specific probe hybridizations. Equivalent conditions can be determined by varying one or more of the parameters given as an example, as known in the art, while maintaining a similar degree of identity or similarity between the target nucleotide sequence and the primer or probe used.

The term “isolated” is used herein to indicate that the material in question exists in a physical milieu distinct from that in which it occurs in nature, and thus is altered “by the hand of man” from its natural state. For example, an isolated nucleic acid of the invention may be substantially isolated with respect to the complex cellular milieu in which it naturally occurs. In some instances, the isolated material will form part of a composition (for example, a crude extract containing other substances), buffer system or reagent mix. In other circumstance, the material may be purified to essential homogeneity, for example as determined by PAGE or column chromatography such as HPLC. Preferably, an isolated nucleic acid comprises at least about 50, 80, or 90 percent (on a molar basis) of all macromolecular species present. For example, an isolated polynucleotide could be part of a vector or a composition of matter, or could be contained within a cell, and still be “isolated” because that vector, composition of matter, or particular cell is not the original environment of the polynucleotide. On one hand, the term “isolated” does not refer to genomic or cDNA libraries, whole cell total or mRNA preparations, genomic DNA preparations (including those separated by electrophoresis and transferred onto blots), sheared whole cell genomic DNA preparations or other compositions where the art demonstrates no distinguishing features of the polynucleotide/sequences of the present invention. On the other hand, in consideration of other embodiments of the present invention, specifically the single nucleotide polymorphisms of the present invention, the term “isolated” may refer to genomic or cDNA libraries, whole cell total or mRNA preparations, genomic DNA preparations (including those separated by electrophoresis and transferred onto blots), sheared whole cell genomic DNA preparations. However, the present invention is meant to encompass those compositions where the art demonstrates no distinguishing features of the polynucleotide/sequences of the present invention (e.g., the knowledge that a particular nucleotide position represents a polymorphic site, the knowledge of which allele represents the reference and/or variant nucleotide base, the association of a particular polymorphism with a disease or disorder, wherein such association was not appreciated heretofor, etc.).

On one hand, and in specific embodiments, the polynucleotides of the invention are at least 15, at least 30, at least 50, at least 100, at least 125, at least 500, or at least 1000 continuous nucleotides but are less than or equal to 300 kb, 200 kb, 100 kb, 50 kb, 15 kb, 10 kb, 7.5 kb, 5 kb, 2.5 kb, 2.0 kb, or 1 kb, in length. In a further embodiment, polynucleotides of the invention comprise a portion of the coding sequences, as disclosed herein, but do not comprise all or a portion of any intron. In another embodiment, the polynucleotides comprising coding sequences do not contain coding sequences of a genomic flanking gene (i.e., 5′ or 3′ to the gene of interest in the genome). In other embodiments, the polynucleotides of the invention do not contain the coding sequence of more than 1000, 500, 250, 100, 50, 25, 20, 15, 10, 5, 4, 3, 2, or 1 genomic flanking gene(s).

On the other hand, and in specific embodiments, the polynucleotides of the invention are at least 15, at least 30, at least 50, at least 100, at least 125, at least 500, or at least 1000 continuous nucleotides but are less than or equal to 300 kb, 200 kb, 100 kb, 50 kb, 15 kb, 10 kb, 7.5 kb, 5 kb, 2.5 kb, 2.0 kb, or 1 kb, in length. In a further embodiment, polynucleotides of the invention comprise a portion of the coding sequences, comprise a portion of non-coding sequences, comprise a portion of an intron sequence, etc., as disclosed herein. In another embodiment, the polynucleotides comprising coding sequences may correspond to a genomic sequence flanking a gene (i.e., 5′ or 3′ to the gene of interest in the genome). In other embodiments, the polynucleotides of the invention may contain the non-coding sequence of more than 1000, 500, 250, 100, 50, 25, 20, 15, 10, 5, 4, 3, 2, or 1 genomic flanking gene(s).

As used herein, a “polynucleotide” refers to a molecule having a nucleic acid sequence contained in SEQ ID NO:5, 7, 9, 11, 13, 15, 17, 19, 21, 23, 25, 27, 29, 31, 33, 35, 37, 39, 41, 43, 45, 47, 49, and/or 603. For example, the polynucleotide can contain the nucleotide sequence of the full length cDNA sequence, including the 5′ and 3′ untranslated sequences, the coding region, with or without a signal sequence, the secreted protein coding region, as well as fragments, epitopes, domains, and variants of the nucleic acid sequence. Moreover, as used herein, a “polypeptide” refers to a molecule having the translated amino acid sequence generated from the polynucleotide as broadly defined.

Unless otherwise indicated, all nucleotide sequences determined by sequencing a DNA molecule herein were determined using an automated DNA sequencer (such as the Model 373, preferably a Model 3700, from Applied Biosystems, Inc.), and all amino acid sequences of polypeptides encoded by DNA molecules determined herein were predicted by translation of a DNA sequence determined above. Therefore, as is known in the art for any DNA sequence determined by this automated approach, any nucleotide sequence determined herein may contain some errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion.

Using the information provided herein, such as the nucleotide sequence provided as SEQ ID NO:5, 7, 9, 11, 13, 15, 17, 19, 21, 23, 25, 27, 29, 31, 33, 35, 37, 39, 41, 43, 45, 47, 49, and/or 603, a nucleic acid molecule of the present invention encoding a polypeptide of the present invention may be obtained using standard cloning and screening procedures, such as those for cloning cDNAs using mRNA as starting material.

A “polynucleotide” of the present invention also includes those polynucleotides capable of hybridizing, under stringent hybridization conditions, to sequences contained in SEQ ID NO:5, 7, 9, 11, 13, 15, 17, 19, 21, 23, 25, 27, 29, 31, 33, 35, 37, 39, 41, 43, 45, 47, 49, and/or 603, or the complement thereof. “Stringent hybridization conditions” refers to an overnight incubation at 42 degree C in a solution comprising 50% formamide, 5×SSC (750 mM NaCl, 75 mM trisodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 65 degree C.

Also contemplated are nucleic acid molecules that hybridize to the polynucleotides of the present invention at lower stringency hybridization conditions. Changes in the stringency of hybridization and signal detection are primarily accomplished through the manipulation of formamide concentration (lower percentages of formamide result in lowered stringency); salt conditions, or temperature. For example, lower stringency conditions include an overnight incubation at 37 degree C. in a solution comprising 6×SSPE (20×SSPE=3M NaCl; 0.2M NaH2PO4; 0.02M EDTA, pH 7.4), 0.5% SDS, 30% formamide, 100 ug/ml salmon sperm blocking DNA; followed by washes at 50 degree C. with 1×SSPE, 0.1% SDS. In addition, to achieve even lower stringency, washes performed following stringent hybridization can be done at higher salt concentrations (e.g. 5×SSC).

Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

Of course, a polynucleotide which hybridizes only to polyA+ sequences (such as any 3′ terminal polyA+ tract of a cDNA shown in the sequence listing), or to a complementary stretch of T (or U) residues, would not be included in the definition of “polynucleotide,” since such a polynucleotide would hybridize to any nucleic acid molecule containing a poly (A) stretch or the complement thereof (e.g., practically any double-stranded cDNA clone generated using oligo dT as a primer).

The polynucleotide of the present invention can be composed of any polyribonucleotide or polydeoxribonucleotide, which may be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the polynucleotide can be composed of triple-stranded regions comprising RNA or DNA or both RNA and DNA. A polynucleotide may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

The polypeptide of the present invention can be composed of amino acids joined to each other by peptide bonds or modified peptide bonds, i.e., peptide isosteres, and may contain amino acids other than the 20 gene-encoded amino acids. The polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins—Structure and Molecular Properties, 2nd Ed., T. E. Creighton, W. H. Freeman and Company, New York (1993); Posttranslational Covalent Modification of Proteins, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992).)

“SEQ ID NO:5, 7, 9, 11, 13, 15, 17, 19, 21, 23, 25, 27, 29, 31, 33, 35, 37, 39, 41, 43, 45, 47, 49, and/or 603” refers to a polynucleotide sequence while “SEQ ID NO:6, 8, 10, 12, 14, 16, 18, 20, 22, 24, 26, 28, 30, 32, 34, 36, 38, 40, 42, 44, 46, 48, 50, and/or 604” refers to a polypeptide sequence, both sequences identified by an integer specified in Table I, and/or in Table IV, V, or VI.

“A polypeptide having biological activity” refers to polypeptides exhibiting activity similar, but not necessarily identical to, an activity of a polypeptide of the present invention, including mature forms, as measured in a particular biological assay, with or without dose dependency. In the case where dose dependency does exist, it need not be identical to that of the polypeptide, but rather substantially similar to the dose-dependence in a given activity as compared to the polypeptide of the present invention (i.e., the candidate polypeptide will exhibit greater activity or not more than about 25-fold less and, preferably, not more than about tenfold less activity, and most preferably, not more than about three-fold less activity relative to the polypeptide of the present invention.)

The term “organism” as referred to herein is meant to encompass any organism referenced herein, though preferably to eukaryotic organisms, more preferably to manunals, and most preferably to humans.

The present invention encompasses the identification of proteins, nucleic acids, or other molecules, that bind to polypeptides and polynucleotides of the present invention (for example, in a receptor-ligand interaction). The polynucleotides of the present invention can also be used in interaction trap assays (such as, for example, that described by Ozenberger and Young (Mol Endocrinol., 9(10): 1321-9, (1995); and Ann. N. Y. Acad. Sci., 7;766:279-81, (1995)).

The polynucleotide and polypeptides of the present invention are useful as probes for the identification and isolation of full-length cDNAs and/or genomic DNA which correspond to the polynucleotides of the present invention, as probes to hybridize and discover novel, related DNA sequences, as probes for positional cloning of this or a related sequence, as probe to “subtract-out” known sequences in the process of discovering other novel polynucleotides, as probes to quantify gene expression, and as probes for microarrays.

In addition, polynucleotides and polypeptides of the present invention may comprise one, two, three, four, five, six, seven, eight, or more membrane domains.

Also, in preferred embodiments the present invention provides methods for further refining the biological function of the polynucleotides and/or polypeptides of the present invention.

Specifically, the invention provides methods for using the polynucleotides and polypeptides of the invention to identify orthologs, homologs, paralogs, variants, and/or allelic variants of the invention. Also provided are methods of using the polynucleotides and polypeptides of the invention to identify the entire coding region of the invention, non-coding regions of the invention, regulatory sequences of the invention, and secreted, mature, pro-, prepro-, forms of the invention (as applicable).

In preferred embodiments, the invention provides methods for identifying the glycosylation sites inherent in the polynucleotides and polypeptides of the invention, and the subsequent alteration, deletion, and/or addition of said sites for a number of desirable characteristics which include, but are not limited to, augmentation of protein folding, inhibition of protein aggregation, regulation of intracellular trafficking to organelles, increasing resistance to proteolysis, modulation of protein antigenicity, and mediation of intercellular adhesion.

In further preferred embodiments, methods are provided for evolving the polynucleotides and polypeptides of the present invention using molecular evolution techniques in an effort to create and identify novel variants with desired structural, functional, and/or physical characteristics.

As used herein the terms “modulate” or “modulates” refer to an increase or decrease in the amount, quality or effect of a particular activity, DNA, RNA, or protein. The definition of “modulate” or “modulates” as used herein is meant to encompass agonists and/or antagonists of a particular activity, DNA, RNA, or protein.

The present invention further provides for other experimental methods and procedures currently available to derive functional assignments. These procedures include but are not limited to spotting of clones on arrays, micro-array technology, PCR based methods (e.g., quantitative PCR), anti-sense methodology, gene knockout experiments, and other procedures that could use sequence information from clones to build a primer or a hybrid partner.

### Polynucleotides and Polypeptides of the Invention

### Features of the Polypeptide Encoded by Gene No:1

The present invention relates to isolated nucleic acid molecules comprising, or alternatively, consisting of all or a portion of the variant allele of the human OATP2, solute carrier family 21 member 6 gene (SNP_ID: PS100s1) (e.g., wherein reference or wildtype OATP2 gene is exemplified by SEQ ID NO:1). Preferred portions are at least 10, preferably at least 20, preferably at least 40, preferably at least 100, contiguous polynucleotides and comprise an “A” at the nucleotide position corresponding to nucleotide 545 of the OATP2 gene, or a portion of SEQ ID NO:5. Alternatively, preferred portions are at least 10, preferably at least 20, preferably at least 40, preferably at least 100, contiguous polynucleotides and comprise a “G” at the nucleotide position corresponding to nucleotide 545 of the OATP2 gene, or a portion of SEQ ID NO:5. The invention further relates to isolated gene products, e.g., polypeptides and/or proteins, which are encoded by a nucleic acid molecule comprising all or a portion of the variant allele of the OATP2 gene.

In one embodiment, the invention relates to a method for predicting the likelihood that an individual will have a disorder associated with an “A” at the nucleotide position corresponding to nucleotide position 545 of SEQ ID NO:5 (or diagnosing or aiding in the diagnosis of such a disorder) comprising the steps of obtaining a DNA sample from an individual to be assessed and determining the nucleotide present at position 545 of SEQ ID NO:5. The presence of an “A” at this position indicates that the individual has a greater likelihood of having a disorder associated therewith than an individual having a “G” at that position, or a greater likelihood of having more severe symptoms.

Conversely, the invention relates to a method for predicting the likelihood that an individual will have a disorder associated with a “G” at the nucleotide position corresponding to nucleotide position 545 of SEQ ID NO:5 (or diagnosing or aiding in the diagnosis of such a disorder) comprising the steps of obtaining a DNA sample from an individual to be assessed and determining the nucleotide present at position 545 of SEQ ID NO:5. The presence of a “G” at this position indicates that the individual has a greater likelihood of having a disorder associated therewith than an individual having an “A” at that position, or a greater likelihood of having more severe symptoms.

Representative disorders which may be detected, diagnosed, identified, treated, prevented, and/or ameliorated by the present invention include, the following, non-limiting diseases and disorders: decreased heptic uptake of administered statins, resistance to beneficial responses of administered statins; resistance to LDL lowering responses to administered statins; resistance to TG lowering responses to administered statins; resistance to HDL elevating responses to administered statins; increased incidence of drug interactions between one or more administered statins; increased incidence of drug and endogenous substance interactions between a statin drug and endogenous substrates of OATP2 including cholate, taurocholate, thyroid hormones T3 and T4, DHEAS, estradiol-17beta-glucuronide, estrone-3-sulfate, prostaglandin E2, thromboxane B2, leukotriene C4, leukotriene E4, and bilirubin and its conjugates; decreased response to HMG-CoA reductase inhibitors; in addition to other disorders referenced herein, which include, for example, any disorder related to high levels of circulating LDL, high levels of TG, and/or low levels of circularing HDL, which include, but are not limited to, cardiovascular diseases, hepatic diseases, angina pectoris, hypertension, heart failure, myocardial infarction, ventricular hypertrophy, vascular diseases, miscrovascular disease, vascular leak syndrome, aneurysm, stroke, embolism, thrombosis, endothelial dysfunction, coronary artery disease, arteriosclerosis, and/or atherosclerosis.

### Features of the Polypeptide Encoded by Gene No:2

The present invention relates to isolated nucleic acid molecules comprising, or alternatively, consisting of all or a portion of the variant allele of the human OATP2, solute carrier family 21 member 6 gene (SNP_ID: PS100s2) (e.g., wherein reference or wildtype OATP2 gene is exemplified by SEQ ID NO:1). Preferred portions are at least 10, preferably at least 20, preferably at least 40, preferably at least 100, contiguous polynucleotides and comprise an “A” at the nucleotide position corresponding to nucleotide 597 of the OATP2 gene, or a portion of SEQ ID NO:7. Alternatively, preferred portions are at least 10, preferably at least 20, preferably at least 40, preferably at least 100, contiguous polynucleotides and comprise a “C” at the nucleotide position corresponding to nucleotide 597 of the OATP2 gene, or a portion of SEQ ID NO:7. The invention further relates to isolated gene products, e.g., polypeptides and/or proteins, which are encoded by a nucleic acid molecule comprising all or a portion of the variant allele of the OATP2 gene.

In one embodiment, the invention relates to a method for predicting the likelihood that an individual will have a disorder associated with an “A” at the nucleotide position corresponding to nucleotide position 597 of SEQ ID NO:7 (or diagnosing or aiding in the diagnosis of such a disorder) comprising the steps of obtaining a DNA sample from an individual to be assessed and determining the nucleotide present at position 597 of SEQ ID NO:7. The presence of an “A” at this position indicates that the individual has a greater likelihood of having a disorder associated therewith than an individual having a “C” at that position, or a greater likelihood of having more severe symptoms.

Conversely, the invention relates to a method for predicting the likelihood that an individual will have a disorder associated with a “C” at the nucleotide position corresponding to nucleotide position 597 of SEQ ID NO:7 (or diagnosing or aiding in the diagnosis of such a disorder) comprising the steps of obtaining a DNA sample from an individual to be assessed and determining the nucleotide present at position 597 of SEQ ID NO:7. The presence of a “C” at this position indicates that the individual has a greater likelihood of having a disorder associated therewith than an individual having an “A” at that position, or a greater likelihood of having more severe symptoms.

The present invention further relates to isolated proteins or polypeptides comprising, or alternatively, consisting of all or a portion of the encoded variant amino acid sequence of the human human OATP2, solute carrier family 21 member 6 polypeptide (e.g., wherein reference or wildtype human OATP2, solute carrier family 21 member 6 polypeptide is exemplified by SEQ ID NO:2). Preferred portions are at least 10, preferably at least 20, preferably at least 40, preferably at least 100, contiguous polypeptides and comprises an “A” at the amino acid position corresponding to amino acid 155 of the OATP2 polypeptide, or a portion of SEQ ID NO:8. Alternatively, preferred portions are at least 10, preferably at least 20, preferably at least 40, preferably at least 100, contiguous polypeptides and comprises a “P” at the amino acid position corresponding to amino acid 155 of the OATP2 protein, or a portion of SEQ ID NO:8. The invention further relates to isolated nucleic acid molecules encoding such polypeptides or proteins, as well as to antibodies that bind to such proteins or polypeptides.

Representative disorders which may be detected, diagnosed, identified, treated, prevented, and/or ameliorated by the present invention include, the following, non-limiting diseases and disorders: decreased heptic uptake of administered statins, resistance to beneficial responses of administered statins; resistance to LDL lowering responses to administered statins; resistance to TG lowering responses to administered statins; resistance to HDL elevating responses to administered statins; increased incidence of drug interactions between one or more administered statins; increased incidence of drug and endogenous substance interactions between a statin drug and endogenous substrates of OATP2 including cholate, taurocholate, thyroid hormones T3 and T4, DHEAS, estradiol-17beta-glucuronide, estrone-3-sulfate, prostaglandin E2, thromboxane B2, leukotriene C4, leukotriene E4, and bilirubin and its conjugates; decreased response to HMG-CoA reductase inhibitors; in addition to other disorders referenced herein, which include, for example, any disorder related to high levels of circulating LDL, high levels of TG, and/or low levels of circularing HDL, which include, but are not limited to, cardiovascular diseases, hepatic diseases, angina pectoris, hypertension, heart failure, myocardial infarction, ventricular hypertrophy, vascular diseases, miscrovascular disease, vascular leak syndrome, aneurysm, stroke, embolism, thrombosis, endothelial dysfunction, coronary artery disease, arteriosclerosis, and/or atherosclerosis.

In preferred embodiments, the following N-terminal OATP2 (SNP_ID: PS100s2) deletion polypeptides are encompassed by the present invention: M1-C691, D2-C691, Q3-C691, N4-C691, Q5-C691, H6-C691, L7-C691, N8-C691, K9-C691, T10-C691, A11-C691, E12-C691, A13-C691, Q14-C691, P15-C691, S16-C691, E17-C691, N18-C691, K19-C691, K20-C691, T21-C691, R22-C691, Y23-C691, C24-C691, N25-C691, G26-C691, L27-C691, K28-C691, M29-C691, F30-C691, L31-C691, A32-C691, A33-C691, L34-C691, S35-C691, L36-C691, S37-C691, F38-C691, I39-C691, A40-C691, K41-C691, T42-C691, L43-C691, G44-C691, A45-C691, I46-C691, I47-C691, M48-C691, K49-C691, S50-C691, S51-C691, I52-C691, I53-C691, H54-C691, I55-C691, E56-C691, R57-C691, R58-C691, F59-C691, E60-C691, I61-C691, S62-C691, S63-C691, S64-C691, L65-C691, V66-C691, G67-C691, F68-C691, 169-C691, D70-C691, G71-C691, S72-C691, F73-C691, E74-C691, I75-C691, G76-C691, N77-C691, L78-C691, L79-C691, V80-C691, I81-C691, V82-C691, F83-C691, V84-C691, S85-C691, Y86-C691, F87-C691, G88-C691, S89-C691, K90-C691, L91-C691, H92-C691, R93-C691, P94-C691, K95-C691, L96-C691, I97-C691, G98-C691, I99-C691, G100-C691, C101-C691, F102-C691, I103-C691, M104-C691, G105-C691, I106-C691, G107-C691, G108-C691, V109-C691, L110-C691, T111-C691, A112-C691, L113-C691, P114-C691, H115-C691, F116-C691, F117-C691, M118-C691, G119-C691, Y120-C691, Y121-C691, R122-C601, Y123-C691, S124-C691, K125-C691, E126-C691, T127-C691, N128-C691, I129-C691, D130-C691, S131-C691, S132-C691, E133-C691, N134-C691, S135-C691, T136-C691, S137-C691, T138-C691, L139-C691, S140-C691, T141-C691, C142-C691, L143-C691, I144-C691, N145-C691, Q146-C691, I147-C691, L148-C691, S149-C691, L150-C691, N151-C691, R152-C691, A153-C691, S154-C691, T155-C691, E156-C691, I157-C691, V158-C691, G159-C691, K160-C691, G161-C691, C162-C691, L163-C691, K164-C691, E165-C691, S166-C691, G167-C691, S168-C691, Y169-C691, M170-C691, W171-C691, I172-C691, Y173-C691, V174-C691, F175-C691, M176-C691, G177-C691, N178-C691, M179-C691, L180-C691, R181-C691, G182-C691, I183-C691, G184-C691, E185-C691, T186-C691, P187-C691, I188-C691, V189-C691, P190-C691, L191-C691, G192-C691, L193-C691, S194-C691, Y195-C691, I196-C691, D197-C691, D198-C691, F199-C691, A200-C691, K201-C691, E202-C691, G203-C691, H204-C691, S205-C691, S206-C691, L207-C691, Y208-C691, L209-C691, G210-C691, I211-C691, L212-C691, N213-C691, A214-C691, I215-C691, A216-C691, M217-C691, I218-C691, G219-C691, P220-C691, I221-C691, I222-C691, G223-C691, F224-C691, T225-C691, L226-C691, G227-C691, S228-C691, L229-C691, F230-C691, S231-C691, K232-C691, M233-C691, Y234-C691, V235-C691, D236-C691, I237-C691, G238-C691, Y239-C691, V240-C691, D241-C691, L242-C691, S243-C691, T244-C691, I245-C691, R246-C691, I247-C691, T248-C691, P249-C691, T250-C691, D251 -C691, S252-C691, R253-C691, W254-C691, V255-C691, G256-C691, A257-C691, W258-C691, W259-C691, L260-C691, N261 -C691, F262-C691, L263-C691, V264-C691, S265-C691, G266-C691, L267-C691, F268-C691, S269-C691, 1270-C691, I271-C691, S272-C691, S273-C691, I274-C691, P275-C691, F276-C691, F277-C691, F278-C691, L279-C691, P280-C691, Q281-C691, T282-C691, P283-C691, N284-C691, K285-C691, P286-C691, Q287-C691, K288-C691, E289-C691, R290-C691, K291 -C691, A292-C691, S293-C691, L294-C691, S295-C691, L296-C691, H297-C691, V298-C691, L299-C691, E300-C691, T301-C691, N302-C691, D303-C691, E304-C691, K305-C691, D306-C691, Q307-C691, T308-C691, A309-C691, N310-C691, L311-C691, T312-C691, N313-C691, Q314-C691, G315-C691, K316-C691, N317-C691, I318-C691, T319-C691, K320-C691, N321-C691, V322-C691, T323-C691, G324-C691, F325-C691, F326-C691, Q327-C691, S328-C691, F329-C691, K330-C691, S331-C691, I332-C691, L333-C691, T334-C691, N335-C691, P336-C691, L337-C691, Y338-C691, V339-C691, M340-C691, F341-C691, V342-C691, L343-C691, L344-C691, T345-C691, L346-C691, L347-C691, Q348-C691, V349-C691, S350-C691. S351-C691, Y352-C691, I353-C691, G354-C691, A355-C691, F356-C691, T357-C691, Y358-C691, V359-C691, F360-C691, K361-C691, Y362-C691, V362-C691, E364-C691, Q365-C691, Q366-C691, Y367-C691, G368-C691, Q369-C691, P370-C691, S371-C691, S372-C691, K373-C691, A374-C691, N375-C691, I376-C691, L377-C691, L378-C691, G379-C691, V380-C691, I381-C691, T382-C691, I383-C691, P384-C691, I385-C691, F386-C691, A387-C691, S388-C691, G389-C691, M390-C691, F391-C691, L392-C691, G393-C691, G394-C691, Y395-C691, I396-C691, I397-C691, K398-C691, K399-C691, F400-C691, K401-C691, L402-C691, N403-C691, T404-C691, V405-C691, G406-C691, I407-C691, A408-C691, K409-C691, F410-C691, S411-C691, C412-C691, F413-C691, T414-C691, A414-C691, V416-C691, M417-C691, S418-C691, L419-C691, S420-C691, F421-C691, Y422-C691, L423-C691, L424-C691, Y425-C691, F426-C691, F427-C691, I428-C691, L429-C691, C430-C691, E431-C691, N432-C691, K433-C691, S434-C691, V435-C691, A436-C691, G437-C691, L438-C691, T439-C691, M440-C691, T441-C691, Y442-C691, D443-C691, G444-C691, N445-C691, N446-C691, P447-C691, V448-C691, T449-C691, S450-C691, H451-C691, R452-C691, D453-C691, V454-C691, P455-C691, L456-C691, S457-C691, Y458-C691, C459-C691, N460-C691, S461-C691, D462-C691, C463-C691, N464-C691, C465-C691, D466-C691, E467-C691, S468-C691, Q469-C691, W470-C691, E471-C691, P472-C691, V473-C691, C474-C691, G475-C691, N476-C691, N477-C691, G478-C691, 1479-C691, T480-C691, Y481-C691, I482-C691, S483-C691, P484-C691, C485-C691, L486-C691, A487-C691, G488-C691, C489-C691, K490-C691, S491-C691, S492-C691, S493-C691, G494-C691, N495-C691, K496-C691, K497-C691, P498-C691, I499-C691, V500-C691, F501-C691, Y502-C691, N503-C691, C504-C691, S505-C691, C506-C691, L507-C691, E508-C691, V509-C691, T510-C691, G511-C691, L512-C691, Q513-C691, N514-C691, R515-C691, N516-C691, Y517-C691, S518-C691, A519-C691, H520-C691, L521-C691, G522-C691, E523-C691, C524-C691, P525-C691, R526-C691, D527-C691, D528-C691, A529-C691, C530-C691, T531-C691, R532-C691, K533-C691, F534-C691, Y535-C691, F536-C691, F537-C691, V538-C691, A539-C691, I540-C691, Q541-C691, V542-C691, L543-C691, N544-C691, L545-C691, F546-C691, F547-C691, S548-C691, A549-C691, L550-C691, G551-C691, G552-C691, T553-C691, S554-C691, H555-C691, V556-C691, M557-C691, L558-C691, I559-C691, V560-C691, K561-C691, I562-C691, V563-C691, Q564-C691, P565-C691, E566-C691, L567-C691, K568-C691, S569-C691, L570-C691, A571-C691, L572-C691, G573-C691, F574-C691, H575-C691, S576-C691, M577-C691, V578-C691, I579-C691, R580-C691, A581-C691, L582-C691, G583-C691, G584-C691, I585-C691, L586-C691, A587-C691, P588-C691, I589-C691, Y590-C691, F591-C691, G592-C691, A593-C691, L594-C691, I595-C691, D596-C691, T597-C691, T598-C691, C599-C691, I600-C691, K601 -C691, W602-C691, S603-C691, T604-C691, N605-C691, N606-C691, C607-C691, G608-C691, T609-C691, R610-C691, G611-C691, S612-C691, C613-C691, R614-C691, T615-C691, Y616-C691, N617-C691, S618-C691, T619-C691, S620-C691, F621-C691, S622-C691, R623-C691, V624-C691, Y625-C691, L626-C691, G627-C691, L628-C691, S629-C691, S630-C691, M631-C691, L632-C691, R633-C691, V634-C691, S635-C691, S636-C691, L637-C691, V638-C691, L639-C691, Y640-C691, I641-C691, I642-C691, L643-C691, I644-C691, Y645-C691, A646-C691, M647-C691, K648-C691, K649-C691, K650-C691, Y651-C691, Q652-C691, E653-C691, K654-C691, D655-C691, I656-C691, N657-C691, A658-C691, S659-C691, E660-C691, N661-C691, G662-C691, S663-C691, V664-C691, M665-C691, D666-C691, E667-C691, A668-C691, N669-C691, L670-C691, E671 -C691, S672-C691, L673-C691, N674-C691, K675-C691, N676-C691, K677-C691, H678-C691, F679-C691, V680-C691, P681 -C691, S682-C691, A683-C691, G684-C691, and/or A685-C691 of SEQ ID NO:8. Polynucleotide sequences encoding these polypeptides are also provided. The present invention also encompasses the use of these N-terminal OATP2 (SNP_ID: PS100s2) deletion polypeptides as immunogenic and/or antigenic epitopes as described elsewhere herein.

In preferred embodiments, the following C-terminal OATP2 (SNP_ID: PS100s2) deletion polypeptides are encompassed by the present invention: M1-C691, M1-H690, M1-T689, M1-E688, M1-S687, M1-D686, M1-A685, M1-G684, M1-A683, M1-S682, M1-P681, M1-V680, M1-F679, M1-H678, M1-K677, M1-N676, M1-K675, M1-N674, M1-L673, M1-S672, M1-E671, M1-L670, M1-N669, M1-A668, M1-E667, M1-D666, M1-M665, M1-V664, M1-S663, M1-G662, M1-N661, M1-E660, M1-S659, M1-A658, M1-N657, M1-1656, M1-D655, M1-K654, M1-E653, M1-Q652, M1-Y651, M1-K650, M1-K649, M1-K648, M1-M647, M1-A646, M1-Y645, M1-1644, M1-L643, M1-1642, M1-1641, M1-Y640, M1-L639, M1-V638, M1-L637, M1-S636, M1-S635, M1-V634, M1-R633, M1-L632, M1-M631, M1-S630, M1-S629, M1-L628, M1-G627, M1-L626, M1-Y625, M1-V624, M1-R623, M1-S622, M1-F621, M1-S620, M1-T619, M1-S618, M1-N617, M1-Y616, M1-T615, M1-R614, M1-C613, M1-S612, M1-G611, M1-R610, M1-T609, M1-G608, M1-C607, M1-N606, M1-N605, M1-T604, M1-S603, M1-W602, M1-K601, M1-I600, M1-C599, M1-T598, M1-T597, M1-D596, M1-1595, M1-L594, M1-A593, M1-G592, M1-F591, M1-Y590, M1-1589, M1-P588, M1-A587, M1-L586, M1-I585, M1-G584, M1-G583, M1-L582, M1-A581, M1-R580, M1-I579, M1-V578, M1-M577, M1-S576, M1-H575, M1-F574, M1-G573, M1-L572, M1-A571, M1-L570, M1-S569, M1-K568, M1-L567, M1-E566, M1-P565, M1-Q564, M1-V563, M1-I562, M1-K561, M1-V560, M1-I559, M1-L558, M1-M557, M1-V556, M1-H555, M1-S554, M1-T553, M1-G552, M1-G551, M1-L550, M1-A549, M1-S548, M1-F547, M1-F546, M1-L545, M1-N544, M1-L543, M1-V542, M1-Q541, M1-I540, M1-A539, M1-V538, M1-F537, M1-F536, M1-Y535, M1-F534, M1-K533, M1-R532, M1-T531, M1-C530, M1-A529, M1-D528, M1-D527, M1-R526, M1-P525, M1-C524, M1-E523, M1-G522, M1-L521, M1-H520, M1-A519, M1-S518, M1-Y517, M1-N516, M1-R515, M1-N514, M1-Q513, M1-L512, M1-G511, M1-T510, M1-V509, M1-E508, M1-L507, M1-C506, M1-S505, M1-C504, M1-N503, M1-Y502, M1-F501, M1-V500, M1-I499, M1-P498, M1-K497, M1-K496, M1-N495, M1-G494, M1-S493, M1-S492, M1-S491, M1-K490, M1-C489, M1-G488, M1-A487, M1-L486, M1-C485, M1-P484, M1-S483, M1-I482, M1-Y481, M1-T480, M1-I479, M1-G478, M1-N477, M1-N476, M1-G475, M1-C474, M1-V473, M1-P472, M1-E471, M1-W470, M1-Q469, M1-S468, M1-E467, M1-D466, M1-C465, M1-N464, M1-C463, M1-D462, M1-S461, M1-N460, M1-C459, M1-Y458, M1-S457, M1-L456, M1-P455, M1-V454, M1-D453, M1-R452, M1-H451, M1-S450, M1-T449, M1-V448, M1-P447, M1-N446, M1-N445, M1-G444, M1-D443, M1-Y442, M1-T441, M1-M440, M1-T439, M1-L438, M1-G437, M1-A436, M1-V435, M1-S434, M1-K433, M1-N432, M1-E431, M1-C430, M1-L429, M1-I428, M1-F427, M1-F426, M1-Y425, M1-L424, M1-L423, M1-Y422, M1-F421, M1-S420, M1-L419, M1-S418, M1-M417, M1-V416, M1-A415, M1-T414, M1-F413, M1-C412, M1-S411, M1-F410, M1-K409, M1-A408, M1-I407, M1-G406, M1-V405, M1-T404, M1-N403, M1-L402, M1-K401, M1-F400, M1-K399, M1-K398, M1-I397, M1-I396, M1-Y395, M1-G394, M1-G393, M1-L392, M1-F391, M1-M390, M1-G389, M1-S388, M1-A387, M1-F386, M1-I385, M1-P384, M1-I383, M1-T382, M1-I381, M1-V380, M1-G379, M1-L378, M1-L377, M1-I376, M1-N375, M1-A374, M1-K373, M1-S372, M1-S371, M1-P370, M1-Q369, M1-G368, M1-Y367, M1-Q366, M1-Q365, M1-E364, M1-V363, M1-Y362, M1-K361, M1-F360, M1-V359, M1-Y358, M1-T357, M1-F356, M1-A355, M1-G354, M1-I353, M1-Y352, M1-S351, M1-S350, M1-V349, M1-Q348, M1-L347, M1-L346, M1-T345, M1-L344, M1-L343, M1-V342, M1-F341, M1-M340, M1-V339, M1-Y338, M1-L337, M1-P336, M1-N335, M1-T334, M1-L333, M1-I332, M1-S331, M1-K330, M1-F329, M1-S328, M1-Q327, M1-F326, M1-F325, M1-G324, M1-T323, M1-V322, M1-N321, M1-K320, M1-T319, M1-I318, M1-N317, M1-K316, M1-G315, M1-Q314, M1-N313, M1-T312, M1-L311, M1-N310, M1-A309, M1-T308, M1-Q307, M1-D306, M1-K305, M1-E304, M1-D303, M1-N302, M1-T301, M1-E300, M1-L299, M1-V298, M1-H297, M1-L296, M1-S295, M1-L294, M1-S293, M1-A292, M1-K291, M1-R290, M1-E289, M1-K288, M1-Q287, M1-P286, M1-K285, M1-N284, M1-P283, M1-T282, M1-Q281, M1-P280, M1-L279, M1-F278, M1-F277, M1-F276, M1-P275, M1-I274, M1-S273, M1-S272, M1-I271, M1-I270, M1-S269, M1-F268, M1-L267, M1-G266, M1-S265, M1-V264, M1-L263, M1-F262, M1-N261, M1-L260, M1-W259, M1-W258, M1-A257, M1-G256, M1-V255, M1-W254, M1-R253, M1-S252, M1-D251, M1-T250, M1-P249, M1-T248, M1-I247, M1-R246, M1-I245, M1-T244, M1-S243, M1-L242, M1-D241, M1-V240, M1-Y239, M1-G238, M1-I237, M1-D236, M1-V235, M1-Y234, M1-M233, M1-K232, M1-S231, M1-F230, M1-L229, M1-S228, M1-G227, M1-L226, M1-T225, M1-F224, M1-G223, M1-I222, M1-I221, M1-P220, M1-G219, M1-I218, M1-M217, M1-A216, M1-I215, M1-A214, M1-N213, M1-L212, M1-I211, M1-G210, M1-L209, M1-Y208, M1-L207, M1-S206, M1-S205, M1-H204, M1-G203, M1-E202, M1-K201, M1-A200, M1-F199, M1-D198, M1-D197, M1-I196, M1-Y195, M1-S194, M1-L193, M1-G192, M1-L191, M1-P190, M1-V189, M1-I188, M1-P187, M1-T186, M1-E185, M1-G184, M1-I183, M1-G182, M1-R181, M1-L180, M1-M179, M1-N178, M1-G177, M1-M176, M1-F175, M1-V174, M1-Y173, M1-I172, M1-W171, M1-M170, M1-Y169, M1-S168, M1-G167, M1-S166, M1-E165, M1-K164, M1-L163, M1-C162, M1-G161, M1-K160, M1-G159, M1-V158, M1-I157, M1-E156, M1-T155, M1-S154, M1-A153, M1-R152, M1-N151, M1-L150, M1-S149, M1-L148, M1-I147, M1-Q146, M1-N145, M1-I144, M1-L143, M1-C142, M1-T141, M1-S140, M1-L139, M1-T138, M1-S137, M1-T136, M1-S135, M1-N134, M1-E133, M1-S132, M1-S131, M1-D130, M1-I129, M1-N128, M1-T127, M1-E126, M1-K125, M1-S124, M1-Y123, M1-R122, M1-Y121, M1-Y120, M1-G119, M1-M118, M1-F117, M1-F116, M1-H115, M1-P114, M1-L113, M1-A112, M1-T111, M1-L110, M1-V109, M1-G108, M1-G107, M1-1106, M1-G105, M1-M104, M1-I103, M1-F102, M1-C101, M1-G100, M1-I99, M1-G98, M1-I97, M1-L96, M1-K95, M1-P94, M1-R93, M1-H92, M1-L91, M1-K90, M1-S89, M1-G88, M1-F87, M1-Y86, M1-S85, M1-V84, M1-F83, M1-V82, M1-I81, M1-V80, M1-L79, M1-L78, M1-N77, M1-G76, M1-175, M1-E74, M1-F73, M1-S72, M1-G71, M1-D70, M1-I69, M1-F68, M1-G67, M1-V66, M1-L65, M1-S64, M1-S63, M1-S62, M1-I61, M1-E60, M1-F59, M1-R58, M1-R57, M1-E56, M1-I55, M1-H54, M1-I53, M1-I52, M1-S51, M1-S50, M1-K49, M1-M48, M1-I47, M1-I46, M1-A45, M1-G44, M1-L43, M1-T42, M1-K41, M1-A40, M1-I39, M1-F38, M1-S37, M1-L36, M1-S35, M1-L34, M1-A33, M1-A32, M1-L31, M1-F30, M1-M29, M1-K28, M1-L27, M1-G26, M1-N25, M1-C24, M1-Y23, M1-R22, M1-T21, M1-K20, M1-K19, M1-N18, M1-E17, M1-S16, M1-P15, M1-Q14, M1-A13, M1-E12, M1-A11, M1-T10, M1-K9, M1-N8, and/or M1-L7 of SEQ ID NO:8. Polynucleotide sequences encoding these polypeptides are also provided. The present invention also encompasses the use of these C-terminal OATP2 (SNP_ID: PS100s2) deletion polypeptides as immunogenic and/or antigenic epitopes as described elsewhere herein.

Alternatively, preferred polypeptides of the present invention may comprise polypeptide sequences corresponding to, for example, internal regions of the OATP2 (SNP_ID: PS100s2) polypeptide (e.g., any combination of both N- and C-terminal OATP2 (SNP_ID: PS100s2) polypeptide deletions) of SEQ ID NO:8. For example, internal regions could be defined by the equation: amino acid NX to amino acid CX, wherein NX refers to any N-terminal deletion polypeptide amino acid of OATP2 (SNP_ID: PS100s2) (SEQ ID NO:8), and where CX refers to any C-terminal deletion polypeptide amino acid of OATP2 (SNP_ID: PS100s2) (SEQ ID NO:8). Polynucleotides encoding these polypeptides are also provided. The present invention also encompasses the use of these polypeptides as an immunogenic and/or antigenic epitope as described elsewhere herein. Preferably, the resulting deletion polypeptide comprises the polypeptide polymorphic loci identified elsewhere herein for OATP2 (SNP_ID: PS100s2), and more preferably comprises the polypeptide polymorphic allele identified elsewhere herein for OATP2 (SNP_ID: PS100s2).

### Features of the Polypeptide Encoded by Gene No:3

The present invention relates to isolated nucleic acid molecules comprising, or alternatively, consisting of all or a portion of the variant allele of the human OATP2, solute carrier family 21 member 6 gene (SNP_ID: PS100s9) (e.g., wherein reference or wildtype OATP2 gene is exemplified by SEQ ID NO:1). Preferred portions are at least 10, preferably at least 20, preferably at least 40, preferably at least 100, contiguous polynucleotides and comprise a “T at the nucleotide position corresponding to nucleotide 522 of the OATP2 gene, or a portion of SEQ ID NO:9. Alternatively, preferred portions are at least 10, preferably at least 20, preferably at least 40, preferably at least 100, contiguous polynucleotides and comprise a “G” at the nucleotide position corresponding to nucleotide 522 of the OATP2 gene, or a portion of SEQ ID NO:9. The invention further relates to isolated gene products, e.g., polypeptides and/or proteins, which are encoded by a nucleic acid molecule comprising all or a portion of the variant allele of the OATP2 gene.

In one embodiment, the invention relates to a method for predicting the likelihood that an individual will have a disorder associated with a “T” at the nucleotide position corresponding to nucleotide position 522 of SEQ ID NO:9 (or diagnosing or aiding in the diagnosis of such a disorder) comprising the steps of obtaining a DNA sample from an individual to be assessed and determining the nucleotide present at position 522 of SEQ ID NO:9. The presence of a “T” at this position indicates that the individual has a greater likelihood of having a disorder associated therewith than an individual having a “G” at that position, or a greater likelihood of having more severe symptoms.

Conversely, the invention relates to a method for predicting the likelihood that an individual will have a disorder associated with a “G” at the nucleotide position corresponding to nucleotide position 522 of SEQ ID NO:9 (or diagnosing or aiding in the diagnosis of such a disorder) comprising the steps of obtaining a DNA sample from an individual to be assessed and determining the nucleotide present at position 522 of SEQ ID NO:9. The presence of a “G” at this position indicates that the individual has a greater likelihood of having a disorder associated therewith than an individual having a “T” at that position, or a greater likelihood of having more severe symptoms.

The present invention further relates to isolated proteins or polypeptides comprising, or alternatively, consisting of all or a portion of the encoded variant amino acid sequence of the human human OATP2, solute carrier family 21 member 6 polypeptide (e.g., wherein reference or wildtype human OATP2, solute carrier family 21 member 6 polypeptide is exemplified by SEQ ID NO:2). Preferred portions are at least 10, preferably at least 20, preferably at least 40, preferably at least 100, contiguous polypeptides and comprises a “T” at the amino acid position corresponding to amino acid 130 of the OATP2 polypeptide, or a portion of SEQ ID NO:10. Alternatively, preferred portions are at least 10, preferably at least 20, preferably at least 40, preferably at least 100, contiguous polypeptides and comprises a “P” at the amino acid position corresponding to amino acid 130 of the OATP2 protein, or a portion of SEQ ID NO:10. The invention further relates to isolated nucleic acid molecules encoding such polypeptides or proteins, as well as to antibodies that bind to such proteins or polypeptides.

Representative disorders which may be detected, diagnosed, identified, treated, prevented, and/or ameliorated by the present invention include, the following, non-limiting diseases and disorders: decreased heptic uptake of administered statins, resistance to beneficial responses of administered statins; resistance to LDL lowering responses to administered statins; resistance to TG lowering responses to administered statins; resistance to HDL elevating responses to administered statins; increased incidence of drug interactions between one or more administered statins; increased incidence of drug and endogenous substance interactions between a statin drug and endogenous substrates of OATP2 including cholate, taurocholate, thyroid hormones T3 and T4, DHEAS, estradiol-17beta-glucuronide, estrone-3-sulfate, prostaglandin E2, thromboxane B2, leukotriene C4, leukotriene E4, and bilirubin and its conjugates; decreased response to HMG-CoA reductase inhibitors; in addition to other disorders referenced herein, which include, for example, any disorder related to high levels of circulating LDL, high levels of TG, and/or low levels of circularing HDL, which include, but are not limited to, cardiovascular diseases, hepatic diseases, angina pectoris, hypertension, heart failure, myocardial infarction, ventricular hypertrophy, vascular diseases, miscrovascular disease, vascular leak syndrome, aneurysm, stroke, embolism, thrombosis, endothelial dysfunction, coronary artery disease, arteriosclerosis, and/or atherosclerosis.

In preferred embodiments, the following N-terminal OATP2 (SNP_ID: PS100s9) deletion polypeptides are encompassed by the present invention: M1-C691, D2-C691, Q3-C691, N4-C691, Q5-C691, H6-C691, L7-C691, N8-C691, K9-C691, T10-C691, A11-C691, E12-C691, A13-C691, Q14-C691, P15-C691, S16-C691, E17-C691, N18-C691, K19-C691, K20-C691, T21-C691, R22-C691, Y23-C691, C24-C691, N25-C691, G26-C691, L27-C691, K28-C691, M29-C691, F30-C691, L31-C691, A32-C691, A33-C691, L34-C691, S35-C691, L36-C691, S37-C691, F38-C691, I39-C691, A40-C691, K41-C691, T42-C691, L43-C691, G44-C691, A45-C691, I46-C691, I47-C691, M48-C691, K49-C691, S50-C691, S51-C691, I52-C691, I53-C691, H54-C691, I55-C691, E56-C691, R57-C691, R58-C691, F59-C691, E60-C691, I61-C691, S62-C691, S63-C691, S64-C691, L65-C691, V66-C691, G67-C691, F68-C691, I69-C691, D70-C691, G71-C691, S72-C691, F73-C691, E74-C691, I75-C691, G76-C691, N77-C691, L78-C691, L79-C691, V80-C691, I81-C691, V82-C691, F83-C691, V84-C691, S85-C691, Y86-C691, F87-C691, G88-C691, S89-C691, K90-C691, L91-C691, H92-C691, R93-C691, P94-C691, K95-C691, L96-C691, I97-C691, G98-C691, I99-C691, G100-C691, C101-C691, F102-C691, I103-C691, M104-C691, G105-C691, I106-C691, G107-C691, G108-C691, V109-C691, L110-C691, T111-C691, A112-C691, L113-C691, P114-C691, H115-C691, F116-C691, F117-C691, M118-C691, G119-C691, Y120-C691, Y121-C691, R122-C691, Y123-C691, S124-C691, K125-C691, E126-C691, T127-C691, N128-C691, I129-C691, and/or Y130-C691 of SEQ ID NO:10. Polynucleotide sequences encoding these polypeptides are also provided. The present invention also encompasses the use of these N-terminal OATP2 (SNP_ID: PS100s9) deletion polypeptides as immunogenic and/or antigenic epitopes as described elsewhere herein.

In preferred embodiments, the following C-terminal OATP2 (SNP_ID: PS100s9) deletion polypeptides are encompassed by the present invention: M1-C691, M1-H690, M1-T689, M1-E688, M1-S687, M1-D686, M1-A685, M1-G684, M1-A683, M1-S682, M1-P681, M1-V680, M1-F679, M1-H678, M1-K677, M1-N676, M1-K675, M1-N674, M1-L673, M1-S672, M1-E671, M1-L670, M1-N669, M1-A668, M1-E667, M1-D666, M1-M665, M1-V664, M1-S663, M1-G662, M1-N661, M1-E660, M1-S659, M1-A658, M1-N657, M1-1656, M1-D655, M1-K654, M1-E653, M1-Q652, M1-Y651, M1-K650, M1-K649, M1-K648, M1-M647, M1-A646, M1-Y645, M1-I644, M1-L643, M1-I642, M1-I641, M1-Y640, M1-L639, M1-V638, M1-L637, M1-S636, M1-S635, M1-V634, M1-R633, M1-L632, M1-M631, M1-S630, M1-S629, M1-L628, M1-G627, M1-L626, M1-Y625, M1-V624, M1-R623, M1-S622, M1-F621, M1-S620, M1-T619, M1-S618, M1-N617, M1-Y616, M1-T615, M1-R614, M1-C613, M1-S612, M1-G611, M1-R610, M1-T609, M1-G608, M1-C607, M1-N606, M1-N605, M1-T604, M1-S603, M1-W602, M1-K601, M1-I600, M1-C599, M1-T598, M1-T597, M1-D596, M1-I595, M1-L594, M1-A593, M1-G592, M1-F591, M1-Y590, M1-I589, M1-P588, M1-A587, M1-L586, M1-I585, M1-G584, M1-G583, M1-L582, M1-A581, M1-R580, M1-I579, M1-V578, M1-M577, M1-S576, M1-H575, M1-F574, M1-G573, M1-L572, M1-A571, M1-L570, M1-S569, M1-K568, M1-L567, M1-E566, M1-P565, M1-Q564, M1-V563, M1-I562, M1-K561, M1-V560, M1-I559, M1-L558, M1-M557, M1-V556, M1-H555, M1-S554, M1-T553, M1-G552, M1-G551, M1-L550, M1-A549, M1-S548, M1-F547, M1-F546, M1-L545, M1-N544, M1-L543, M1-V542, M1-Q541, M1-I540, M1-A539, M1-V538, M1-F537, M1-F536, M1-Y535, M1-F534, M1-K533, M1-R532, M1-T531, M1-C530, M1-A529, M1-D528, M1-D527, M1-R526, M1-P525, M1-C524, M1-E523, M1-G522, M1-L521, M1-H520, M1-A519, M1-S518, M1-Y517, M1-N516, M1-R515, M1-N514, M1-Q513, M1-L512, M1-G511, M1-T510, M1-V509, M1-E508, M1-L507, M1-C506, M1-S505, M1-C504, M1-N503, M1-Y502, M1-F501, M1-V500, M1-I499, M1-P498, M1-K497, M1-K496, M1-N495, M1-G494, M1-S493, M1-S492, M1-S491, M1-K490, M1-C489, M1-G488, M1-A487, M1-L486, M1-C485, M1-P484, M1-S483, M1-I482, M1-Y481, M1-T480, M1-I479, M1-G478, M1-N477, M1-N476, M1-G475, M1-C474; M1-V473, M1-P472, M1-E471, M1-W470, M1-Q469, M1-S468, M1-E467, M1-D466, M1-C465, M1-N464, M1-C463, M1-D462, M1-S461, M1-N460, M1-C459, M1-Y458, M1-S457, M1-L456, M1-P455, M1-V454, M1-D453, M1-R452, M1-H451, M1-S450, M1-T449, M1-V448, M1-p447, M1-N446, M1-N445, M1-G444, M1-D443, M1-Y442, M1-T441, M1-M440, M1-T439, M1-L438, M1-G437, M1-A436, M1-V435, M1-S434, M1-K433, M1-N432, M1-E431, M1-C430, M1-L429, M1-I428, M1-F427, M1-F426, M1-Y425, M1-L424, M1-L423, M1-Y422, M1-F421, M1-S420, M1-L419, M1-S418, M1-M417, M1-V416, M1-A415, M1-T414, M1-F413, M1-C412, M1-S411, M1-F410, M1-K409, M1-A408, M1-I407, M1-G406, M1-V405, M1-T404, M1-N403, M1-L402, M1-K401, M1-F400, M1-K399, M1-K398, M1-I397, M1-I396, M1-Y395, M1-G394, M1-G393, M1-L392, M1-F391, M1-M390, M1-G389, M1-S388, M1-A387, M1-F386, M1-I385, M1-P384, M1-I383, M1-T382, M1-I381, M1-V380, M1-G379, M1-L378, M1-L377, M1-I376, M1-N375, M1-A374, M1-K373, M1-S372, M1-S371, M1-P370, M1-Q369, M1-G368, M1-Y367, M1-Q366, M1-Q365, M1-E364, M1-V363, M1-Y362, M1-K361, M1-F360, M1-V359, M1-Y358, M1-T357, M1-F356, M1-A355, M1-G354, M1-I353, M1-Y352, M1-S351, M1-S350, M1-V349, M1-Q348, M1-L347, M1-L346, M1-T345, M1-L344, M1-L343, M1-V342, M1-F341, M1-M340, M1-V339, M1-Y338, M1-L337, M1-P336, M1-N335, M1-T334, M1-L333, M1-I332, M1-S331, M1-K330, M1-F329, M1-S328, M1-Q327, M1-F326, M1-F325, M1-G324, M1-T323, M1-V322, M1-N321, M1-K320, M1-T319, M1-I318, M1-N317, M1-K316, M1-G315, M1-Q314, M1-N313, M1-T312, M1-L311, M1-N310, M1-A309, M1-T308, M1-Q307, M1-D306, M1-K305, M1-E304, M1-D303, M1-N302, M1-T301, M1-E300, M1-L299, M1-V298, M1-H297, M1-L296, M1-S295, M1-L294, M1-S293, M1-A292, M1-K291, M1-R290, M1-E289, M1-K288, M1-Q287, M1-P286, M1-K285, M1-N284, M1-P283, M1-T282, M1-Q281, M1-P280, M1-L279, M1-F278, M1-F277, M1-F276, M1-P275, M1-I274, M1-S273, M1-S272, M1-I271, M1-I270, M1-S269, M1-F268, M1-L267, M1-G266, M1-S265, M1-V264, M1-L263, M1-F262, M1-N261, M1-L260, M1-W259, M1-W258, M1-A257, M1-G256, M1-V255, M1-W254, M1-R253, M1-S252, M1-D251, M1-T250, M1-P249, M1-T248, M1-I247, M1-R246, M1-I245, M1-T244, M1-S243, M1-L242, M1-D241, M1-V240, M1-Y239, M1-G238, M1-I237, M1-D236, M1-V235, M1-Y234, M1-M233, M1-K232, M1-S231, M1-F230, M1-L229, M1-S228, M1-G227, M1-L226, M1-T225, M1-F224, M1-G223, M1-I222, M1-I221, M1-P220, M1-G219, M1-I218, M1-M217, M1-A216, M1-I215, M1-A214, M1-N213, M1-L212, M1-I211, M1-G210, M1-L209, M1-Y208, M1-L207, M1-S206, M1-S205, M1-H204, M1-G203, M1-E202, M1-K201, M1-A200, M1-F199, M1-D198, M1-D197, M1-I196, M1-Y195, M1-S194, M1-L193, M1-G192, M1-L191, M1-P190, M1-V189, M1-I188, M1-P187, M1-T186, M1-E185, M1-G184, M1-I183, M1-G182, M1-R181, M1-L180, M1-M179, M1-N178, M1-G177, M1-M176, M1-F175, M1-V174, M1-Y173, M1-I172, M1-W171, M1-M170, M1-Y169, M1-S168, M1-G167, M1-S166, M1-E165, M1-L164, M1-L163, M1-C162, M1-G161, M1-K160, M1-G159, M1-V158, M1-I157, M1-E156, M1-P155, M1-S154, M1-A153, M1-R152, M1-N151, M1-L150, M1-S149, M1-L148, M1-I147, M1-Q146, M1-N145, M1-I144, M1-L143, M1-C142, M1-T141, M1-S140, M1-L139, M1-T138, M1-S137, M1-T136, M1-S135, M1-N134, M1-E133, M1-S132, M1-S131, and/or M1-Y130 of SEQ ID NO:10. Polynucleotide sequences encoding these polypeptides are also provided. The present invention also encompasses the use of these C-terminal OATP2 (SNP_ID: PS100s9) deletion polypeptides as immunogenic and/or antigenic epitopes as described elsewhere herein.

Alternatively, preferred polypeptides of the present invention may comprise polypeptide sequences corresponding to, for example, internal regions of the OATP2 (SNP_ID: PS100s9) polypeptide (e.g., any combination of both N- and C-terminal OATP2 (SNP_ID: PS100s9) polypeptide deletions) of SEQ ID NO:10. For example, internal regions could be defined by the equation: amino acid NX to amino acid CX, wherein NX refers to any N-terminal deletion polypeptide amino acid of OATP2 (SNP_ID: PS100s9) (SEQ ID NO:10), and where CX refers to any C-terminal deletion polypeptide amino acid of OATP2 (SNP_ID: PS100s9) (SEQ ID NO:10). Polynucleotides encoding these polypeptides are also provided. The present invention also encompasses the use of these polypeptides as an immunogenic and/or antigenic epitope as described elsewhere herein. Preferably, the resulting deletion polypeptide comprises the polypeptide polymorphic loci identified elsewhere herein for OATP2 (SNP_ID: PS100s9), and more preferably comprises the polypeptide polymorphic allele identified elsewhere herein for OATP2 (SNP_ID: PS100s9).

### Features of the Polypeptide Encoded by Gene No:4

The present invention relates to isolated nucleic acid molecules comprising, or alternatively, consisting of all or a portion of the variant allele of the human OATP2, solute carrier family 21 member 6 gene (SNP_ID: PS100s23) (e.g., wherein reference or wildtype OATP2 gene is exemplified by SEQ ID NO:1). Preferred portions are at least 10, preferably at least 20, preferably at least 40, preferably at least 100, contiguous polynucleotides and comprise a “C” at the nucleotide position corresponding to nucleotide 1597 of the OATP2 gene, or a portion of SEQ ID NO:11. Alternatively, preferred portions are at least 10, preferably at least 20, preferably at least 40, preferably at least 100, contiguous polynucleotides and comprise a “G” at the nucleotide position corresponding to nucleotide 1597 of the OATP2 gene, or a portion of SEQ ID NO:11. The invention further relates to isolated gene products, e.g., polypeptides and/or proteins, which are encoded by a nucleic acid molecule comprising all or a portion of the variant allele of the OATP2 gene.

In one embodiment, the invention relates to a method for predicting the likelihood that an individual will have a disorder associated with a “C” at the nucleotide position corresponding to nucleotide position 1597 of SEQ ID NO:11 (or diagnosing or aiding in the diagnosis of such a disorder) comprising the steps of obtaining a DNA sample from an individual to be assessed and determining the nucleotide present at position 1597 of SEQ ID NO:11. The presence of a “C” at this position indicates that the individual has a greater likelihood of having a disorder associated therewith than an individual having a “G” at that position, or a greater likelihood of having more severe symptoms.

Conversely, the invention relates to a method for predicting the likelihood that an individual will have a disorder associated with a “G” at the nucleotide position corresponding to nucleotide position 1597 of SEQ ID NO:11 (or diagnosing or aiding in the diagnosis of such a disorder) comprising the steps of obtaining a DNA sample from an individual to be assessed and determining the nucleotide present at position 1597 of SEQ ID NO:11. The presence of a “G” at this position indicates that the individual has a greater likelihood of having a disorder associated therewith than an individual having a “C” at that position, or a greater likelihood of having more severe symptoms.

The present invention further relates to isolated proteins or polypeptides comprising, or alternatively, consisting of all or a portion of the encoded variant amino acid sequence of the human human OATP2, solute carrier family 21 member 6 polypeptide (e.g., wherein reference or wildtype human OATP2, solute carrier family 21 member 6 polypeptide is exemplified by SEQ ID NO:2). Preferred portions are at least 10, preferably at least 20, preferably at least 40, preferably at least 100, contiguous polypeptides and comprises an “A” at the amino acid position corresponding to amino acid 488 of the OATP2 polypeptide, or a portion of SEQ ID NO:12. Alternatively, preferred portions are at least 10, preferably at least 20, preferably at least 40, preferably at least 100, contiguous polypeptides and comprises a “G” at the amino acid position corresponding to amino acid 488 of the OATP2 protein, or a portion of SEQ ID NO:12. The invention further relates to isolated nucleic acid molecules encoding such polypeptides or proteins, as well as to antibodies that bind to such proteins or polypeptides.

Representative disorders which may be detected, diagnosed, identified, treated, prevented, and/or ameliorated by the present invention include, the following, non-limiting diseases and disorders: decreased heptic uptake of administered statins, resistance to beneficial responses of administered statins; resistance to LDL lowering responses to administered statins; resistance to TG lowering responses to administered statins; resistance to HDL elevating responses to administered statins; increased incidence of drug interactions between one or more administered statins; increased incidence of drug and endogenous substance interactions between a statin drug and endogenous substrates of OATP2 including cholate, taurocholate, thyroid hormones T3 and T4, DHEAS, estradiol-17beta-glucuronide, estrone-3-sulfate, prostaglandin E2, thromboxane B2, leukotriene C4, leukotriene E4, and bilirubin and its conjugates; decreased response to HMG-CoA reductase inhibitors; in addition to other disorders referenced herein, which include, for example, any disorder related to high levels of circulating LDL, high levels of TG, and/or low levels of circularing HDL, which include, but are not limited to, cardiovascular diseases, hepatic diseases, angina pectoris, hypertension, heart failure, myocardial infarction, ventricular hypertrophy, vascular diseases, miscrovascular disease, vascular leak syndrome, aneurysm, stroke, embolism, thrombosis, endothelial dysfunction, coronary artery disease, arteriosclerosis, and/or atherosclerosis.

In preferred embodiments, the following N-terminal OATP2 (SNP_ID: PS100s23) deletion polypeptides are encompassed by the present invention: M1-C691, D2-C691, Q3-C691, N4-C691, Q5-C691, H6-C691, L7-C691, N8-C691, K9-C691, T10-C691, A11-C691, E12-C691, A13-C691, Q14-C691, P15-C691, S16-C691, E17-C691, N18-C691, K19-C691, K20-C691, T21-C691, R22-C691, Y23-C691, C24-C691, N25-C691, G26-C691, L27-C691, K28-C691, M29-C691, F30-C691, L31-C691, A32-C691, A33-C691, L34-C691, S35-C691, L36-C691, S37-C691, F38-C691, I39-C691, A40-C691, K41-C691, T42-C691, L43-C691, G44-C691, A45-C691, I46-C691, I47-C691, M48-C691, K49-C691, S50-C691, S51-C691, I52-C691, I53-C691, H54-C691, I55-C691, E56-C691, R57-C691, R58-C691, F59C691, E60-C691, I61-C691, S62-C691, S63-C691, S64-C691, L65-C691, V66-C691, G67-C691, F68-C691, I69-C691, D70-C691, G71-C691, S72-C691, F73-C691, E74-C691, I75-C691, G76-C691, N77-C691, L78-C691, L79-C691, V80-C691, I81-C691, V82-C691, F83-C691, V84-C691, S85-C691, Y86-C691, F87-C691, G88-C691, S89-C691, K90-C691, L91-C691, H92-C691, R93-C691, P94-C691, K95-C691, L96-C691, I97-C691, G98-C691, I99-C691, G100-C691, C101-C691, F102-C691, I103-C691, M104-C691, G105-C691, I106-C691, G107-C691, G108-C691, V109-C691, L110-C691, T111-C691, A112-C691, L113-C691, P114-C691, H115-C691, F116-C691, F117-C691, M118-C691, G119-C691, Y120-C691, Y121-C691, R122-C691, Y123-C691, S124-C691, K125-C691, E126-C691, T127-C691, N128-C691, I129-C691, and/or Y488-C691 of SEQ ID NO:12. Polynucleotide sequences encoding these polypeptides are also provided. The present invention also encompasses the use of these N-terminal OATP2 (SNP_ID: PS100s23) deletion polypeptides as immunogenic and/or antigenic epitopes as described elsewhere herein.

In preferred embodiments, the following C-terminal OATP2 (SNP_ID: PS100s23) deletion polypeptides are encompassed by the present invention: M1-C691, M1-H690, M1-T689, M1-E688, M1-S687, M1-D686, M1-A685, M1-G684, M1-A683, M1-S682, M1-P681, M1-V680, M1-F679, M1-H678, M1-K677, M1-N676, M1-K675, M1-N674, M1-L673, M1-S672, M1-E671, M1-L670, M1-N669, M1-A668, M1-E667, M1-D666, M1-M665, M1-V664, M1-S663, M1-G662, M1-N661, M1-E660, M1-S659, M1-A658, M1-N657, M1-I656, M1-D655, M1-K654, M1-E653, M1-Q652, M1-Y651, M1-K650, M1-K649, M1-K648, M1-M647, M1-A646, M1-Y645, M1-I644, M1-L643, M1-I642, M1-I641, M1-Y640, M1-L639, M1-V638, M1-L637, M1-S636, M1-S635, M1-V634, M1-R633, M1-L632, M1-M631, M1-S630, M1-S629, M1-L628, M1-G627, M1-L626, M1-Y625, M1-V624, M1-R623, M1-S622, M1-F621, M1-S620, M1-T619, M1-S618, M1-N617, M1-Y616, M1-T615, M1-R614, M1-C613, M1-S612, M1-G611, M1-R610, M1-T609, M1-G608, M1-C607, M1-N606, M1-N605, M1-T604, M1-S603, M1-W602, M1-K601, M1-I600, M1-C599, M1-T598, M1-T597, M1-D596, M1-I595, M1-L594, M1-A593, M1-G592, M1-F591, M1-Y590, M1-I589, M1-P588, M1-A587, M1-L586, M1-I585, M1-G584, M1-G583, M1-L582, M1-A581, M1-R580, M1-I579, M1-V578, M1-M577, M1-S576, M1-H575, M1-F574, M1-G573, M1-L572, M1-A571, M1-L570, M1-S569, M1-K568, M1-L567, M1-E566, M1-P565, M1-Q564, M1-V563, M1-I562, M1-K561, M1-V560, M1-I559, M1-L558, M1-M557, M1-V556, M1-H555, M1-S554, M1-T553, M1-G552, M1-G551, M1-L550, M1-A549, M1-S548, M1-F547, M1-F546, M1-L545, M1-N544, M1-L543, M1-V542, M1-Q541, M1-I540, M1-A539, M1-V538, M1-F537, M1-F536, M1-Y535, M1-F534, M1-K533, M1-R532, M1-T531, M1-C530, M1-A529, M1-D528, M1-D527, M1-R526, M1-P525, M1-C524, M1-E523, M1-G1597, M1-L521, M1-H520, M1-A519, M1-S518, M1-Y517, M1-N516, M1-R515, M1-N514, M1-Q513, M1-L512, M1-G511, M1-T510, M1-V509, M1-E508, M1-L507, M1-C506, M1-S505, M1-C504, M1-N503, M1-Y502, M1-F501, M1-V500, M1-I499, M1-P498, M1-K497, M1-K496, M1-N495, M1-G494, M1-S493, M1-S492, M1-S491, M1-K490, M1-C489, M1-G488, M1-A487, M1-L486, M1-C485, M1-P484, M1-S483, M1-482, M1-Y481, M1-T480, M1-I479, M1-G478, M1-N477, M1-N476, M1-G475, M1-C474, M1-V473, M1-P472, M1-E471, M1-W470, M1-Q469, M1-S468, M1-E467, M1-D466, M1-C465, M1-N464, M1-C463, M1-D462, M1-S461, M1-N460, M1-C459, M1-Y458, M1-S457, M1-L456, M1-P455, M1-V454, M1-D453, M1-R452, M1-H451, M1-S450, M1-T449, M1-V448, M1-P447, M1-N446, M1-N445, M1-G444, M1-D443, M1-Y442, M1-T441, M1-M440, M1-T439, M1-L438, M1-G437, M1-A436, M1-V435, M1-S434, M1-K433, M1-N432, M1-E431, M1-C430, M1-L429, M1-I428, M1-F427, M1-F426, M1-Y425, M1-L424, M1-L423, M1-Y422, M1-F421, M1-S420, M1-L419, M1-S418, M1-M417, M1-V416, M1-A415, M1-T414, M1-F413, M1-C412, M1-S411, M1-F410, M1-K409, M1-A408, M1-I407, M1-G406, M1-V405, M1-T404, M1-N403, M1-L402, M1-K401, M1-F400, M1-K399, M1-K398, M1-I397, M1-I396, M1-Y396, M1-G394, M1-G393, M1-L392, M1-F391, M1-M390, M1-G389, M1-S388, M1-A387, M1-F386, M1-I385, M1-P384, M1-I383, M1-T382, M1-I381, M1-V380, M1-G379, M1-L378, M1-L377, M1-I376, M1-N375, M1-A374, M1-K373, M1-S372, M1-S371, M1-P370, M1-Q369, M1-G368, M1-Y367, M1-Q366, M1-Q365, M1-E364, M1-V363, M1-Y362, M1-K361, M1-F360, M1-V359, M1-Y358, M1-T357, M1-F356, M1-A355, M1-G354, M1-I353, M1-Y352, M1-S351, M1-S350, M1-V349, M1-Q348, M1-L347, M1-L346, M1-T345, M1-L344, M1-L343, M1-V342, M1-F341, M1-M340, M1-V339, M1-Y338, M1-L337, M1-P336, M1-N335, M1-T334, M1-L333, M1-I332, M1-S331, M1-K330, M1-F329, M1-S328, M1-Q327, M1-F326, M1-F325, M1-G324, M1-T323, M1-V322, M1-N321, M1-K320, M1-T319, M1-I318, M1-N317, M1-K316, M1-G315, M1-Q314, M1-N313, M1-T312, M1-L311, M1-N310, M1-A309, M1-T308, M1-Q307, M1-D306, M1-K305, M1-E304, M1-D303, M1-N302, M1-T301, M1-E300, M1-L299, M1-V298, M1-H297, M1-L296, M1-S295, M1-L294, M1-S293, M1-A292, M1-K291, M1-R290, M1-E289, M1-K288, M1-Q287, M1-P286, M1-K285, M1-N284, M1-P283, M1-T282, M1-Q281, M1-P280, M1-L279, M1-F278, M1-F277, M1-F276, M1-P275, M1-I274, M1-S273, M1-S272, M1-I271, M1-I270, M1-S269, M1-F268, M1-L267, M1-G266, M1-S265, M1-V264, M1-L263, M1-F262, M1-N261, M1-L260, M1-W259, M1-W258, M1-A257, M1-G256, M1-V255, M1-W254, M1-R253, M1-S252, M1-D251, M1-T250, M1-P249, M1-T248, M1-I247, M1-R246, M1-I245, M1-T244, M1-S243, M1-L242, M1-D241, M1-V240, M1-Y239, M1-G238, M1-I237, M1-D236, M1-V235, M1-Y234, M1-M233, M1-K232, M1-S231, M1-F230, M1-L229, M1-S228, M1-G227, M1-L226, M1-T225, M1-F224, M1-G223, M1-I222, M1-I221, M1-P220, M1-G219, M1-I218, M1-M217, M1-A216, M1-I215, M1-A214, M1-N213, M1-L212, M1-I211, M1-G210, M1-L209, M1-Y208, M1-L207, M1-S206, M1-S205, M1-H204, M1-G203, M1-E202, M1-K201, M1-A200, M1-F199, M1-D198, M1-D197, M1-I196, M1-Y195, M1-S194, M1-L193, M1-G192, M1-L191, M1-P190, M1-V189, M1-I188, M1-P187, M1-T186, M1-E185, M1-G184, M1-I183, M1-G182, M1-R181, M1-L180, M1-M179, M1-N178, M1-G177, M1-M176, M1-F175, M1-V174, M1-Y173, M1-I172, M1-W171, M1-M170, M1-Y169, M1-S168, M1-G167, M1-S166, M1-E165, M1-K164, M1-L163, M1-C162, M1-G161, M1-K160, M1-G159, M1-V158, M1-I157, M1-E156, M1-P155, M1-S154, M1-A153, M1-R152, M1-N151, M1-L150, M1-S149, M1-L148, M1-I147, M1-Q146, M1-N145, M1-I144, M1-L143, M1-C142, M1-T141, M1-S140, M1-L139, M1-T138, M1-S137, M1-T136, M1-S135, M1-N134, M1-E133, M1-S132, M1-S131, and/or M1-Y488 of SEQ ID NO:12. Polynucleotide sequences encoding these polypeptides are also provided. The present invention also encompasses the use of these C-terminal OATP2 (SNP_ID: PS100s23) deletion polypeptides as immunogenic and/or antigenic epitopes as described elsewhere herein.

Alternatively, preferred polypeptides of the present invention may comprise polypeptide sequences corresponding to, for example, internal regions of the OATP2 (SNP_ID: PS100s23) polypeptide (e.g., any combination of both N- and C-terminal OATP2 (SNP_ID: PS100s23) polypeptide deletions) of SEQ ID NO:12. For example, internal regions could be defined by the equation: amino acid NX to amino acid CX, wherein NX refers to any N-terminal deletion polypeptide amino acid of OATP2 (SNP_ID: PS100s23) (SEQ ID NO:12), and where CX refers to any C-terminal deletion polypeptide amino acid of OATP2 (SNP_ID: PS100s23) (SEQ ID NO:12). Polynucleotides encoding these polypeptides are also provided. The present invention also encompasses the use of these polypeptides as an immunogenic and/or antigenic epitope as described elsewhere herein. Preferably, the resulting deletion polypeptide comprises the polypeptide polymorphic loci identified elsewhere herein for OATP2 (SNP_ID: PS100s23), and more preferably comprises the polypeptide polymorphic allele identified elsewhere herein for OATP2 (SNP_ID: PS100s23).

### Features of the Polypeptide Encoded by Gene No:5

The present invention relates to isolated nucleic acid molecules comprising, or alternatively, consisting of all or a portion of the variant allele of the human OATP2, solute carrier family 21 member 6 gene (SNP_ID: PS100s25) (e.g., wherein reference or wildtype OATP2 gene is exemplified by SEQ ID NO:1). Preferred portions are at least 10, preferably at least 20, preferably at least 40, preferably at least 100, contiguous polynucleotides and comprise an “A” at the nucleotide position corresponding to nucleotide 1382 of the OATP2 gene, or a portion of SEQ ID NO:13. Alternatively, preferred portions are at least 10, preferably at least 20, preferably at least 40, preferably at least 100, contiguous polynucleotides and comprise a “G” at the nucleotide position corresponding to nucleotide 1382 of the OATP2 gene, or a portion of SEQ ID NO:13. The invention further relates to isolated gene products, e.g., polypeptides and/or proteins, which are encoded by a nucleic acid molecule comprising all or a portion of the variant allele of the OATP2 gene.

In one embodiment, the invention relates to a method for predicting the likelihood that an individual will have a disorder associated with an “A” at the nucleotide position corresponding to nucleotide position 1382 of SEQ ID NO:13 (or diagnosing or aiding in the diagnosis of such a disorder) comprising the steps of obtaining a DNA sample from an individual to be assessed and determining the nucleotide present at position 1382 of SEQ ID NO:13. The presence of an “A” at this position indicates that the individual has a greater likelihood of having a disorder associated therewith than an individual having a “G” at that position, or a greater likelihood of having more severe symptoms.

Conversely, the invention relates to a method for predicting the likelihood that an individual will have a disorder associated with a “G” at the nucleotide position corresponding to nucleotide position 1382 of SEQ ID NO:13 (or diagnosing or aiding in the diagnosis of such a disorder) comprising the steps of obtaining a DNA sample from an individual to be assessed and determining the nucleotide present at position 1382 of SEQ ID NO:13. The presence of a “G” at this position indicates that the individual has a greater likelihood of having a disorder associated therewith than an individual having an “A” at that position, or a greater likelihood of having more severe symptoms.

Representative disorders which may be detected, diagnosed, identified, treated, prevented, and/or ameliorated by the present invention include, the following, non-limiting diseases and disorders: decreased heptic uptake of administered statins, resistance to beneficial responses of administered statins; resistance to LDL lowering responses to administered statins; resistance to TG lowering responses to administered statins; resistance to HDL elevating responses to administered statins; increased incidence of drug interactions between one or more administered statins; increased incidence of drug and endogenous substance interactions between a statin drug and endogenous substrates of OATP2 including cholate, taurocholate, thyroid hormones T3 and T4, DHEAS, estradiol-17beta-glucuronide, estrone-3-sulfate, prostaglandin E2, thromboxane B2, leukotriene C4, leukotriene E4, and bilirubin and its conjugates; decreased response to HMG-CoA reductase inhibitors; in addition to other disorders referenced herein, which include, for example, any disorder related to high levels of circulating LDL, high levels of TG, and/or low levels of circularing HDL, which include, but are not limited to, cardiovascular diseases, hepatic diseases, angina pectoris, hypertension, heart failure, myocardial infarction, ventricular hypertrophy, vascular diseases, miscrovascular disease, vascular leak syndrome, aneurysm, stroke, embolism, thrombosis, endothelial dysfunction, coronary artery disease, arteriosclerosis, and/or atherosclerosis.

### Features of the Polypeptide Encoded by Gene No:6

The present invention relates to isolated nucleic acid molecules comprising, or alternatively, consisting of all or a portion of the variant allele of the human OATP2, solute carrier family 21 member 6 gene (SNP_ID: PS100s26) (e.g., wherein reference or wildtype OATP2 gene is exemplified by SEQ ID NO:1). Preferred portions are at least 10, preferably at least 20, preferably at least 40, preferably at least 100, contiguous polynucleotides and comprise a “C” at the nucleotide position corresponding to nucleotide 1334 of the OATP2 gene, or a portion of SEQ ID NO:15. Alternatively, preferred portions are at least 10, preferably at least 20, preferably at least 40, preferably at least 100, contiguous polynucleotides and comprise a “G” at the nucleotide position corresponding to nucleotide 1334 of the OATP2 gene, or a portion of SEQ ID NO:15. The invention further relates to isolated gene products, e.g., polypeptides and/or proteins, which are encoded by a nucleic acid molecule comprising all or a portion of the variant allele of the OATP2 gene.

In one embodiment, the invention relates to a method for predicting the likelihood that an individual will have a disorder associated with a “C” at the nucleotide position corresponding to nucleotide position 1334 of SEQ ID NO:15 (or diagnosing or aiding in the diagnosis of such a disorder) comprising the steps of obtaining a DNA sample from an individual to be assessed and determining the nucleotide present at position 1334 of SEQ ID NO:15. The presence of a “C” at this position indicates that the individual has a greater likelihood of having a disorder associated therewith than an individual having a “G” at that position, or a greater likelihood of having more severe symptoms.

Conversely, the invention relates to a method for predicting the likelihood that an individual will have a disorder associated with a “G” at the nucleotide position corresponding to nucleotide position 1334 of SEQ ID NO:15 (or diagnosing or aiding in the diagnosis of such a disorder) comprising the steps of obtaining a DNA sample from an individual to be assessed and determining the nucleotide present at position 1334 of SEQ ID NO:15. The presence of a “G” at this position indicates that the individual has a greater likelihood of having a disorder associated therewith than an individual having a “C” at that position, or a greater likelihood of having more severe symptoms.

The present invention further relates to isolated proteins or polypeptides comprising, or alternatively, consisting of all or a portion of the encoded variant amino acid sequence of the human human OATP2, solute carrier family 21 member 6 polypeptide (e.g., wherein reference or wildtype human OATP2, solute carrier family 21 member 6 polypeptide is exemplified by SEQ ID NO:2). Preferred portions are at least 10, preferably at least 20, preferably at least 40, preferably at least 100, contiguous polypeptides and comprises a “K” at the amino acid position corresponding to amino acid 400 of the OATP2 polypeptide, or a portion of SEQ ID NO:16. Alternatively, preferred portions are at least 10, preferably at least 20, preferably at least 40, preferably at least 100, contiguous polypeptides and comprises a “F” at the amino acid position corresponding to amino acid 400 of the OATP2 protein, or a portion of SEQ ID NO:16. The invention further relates to isolated nucleic acid molecules encoding such polypeptides or proteins, as well as to antibodies that bind to such proteins or polypeptides.

Representative disorders which may be detected, diagnosed, identified, treated, prevented, and/or ameliorated by the present invention include, the following, non-limiting diseases and disorders: decreased heptic uptake of administered statins, resistance to beneficial responses of administered statins; resistance to LDL lowering responses to administered statins; resistance to TG lowering responses to administered statins; resistance to HDL elevating responses to administered statins; increased incidence of drug interactions between one or more administered statins; increased incidence of drug and endogenous substance interactions between a statin drug and endogenous substrates of OATP2 including cholate, taurocholate, thyroid hormones T3 and T4, DHEAS, estradiol-17beta-glucuronide, estrone-3-sulfate, prostaglandin E2, thromboxane B2, leukotriene C4, leukotriene E4, and bilirubin and its conjugates; decreased response to HMG-CoA reductase inhibitors; in addition to other disorders referenced herein, which include, for example, any disorder related to high levels of circulating LDL, high levels of TG, and/or low levels of circularing HDL, which include, but are not limited to, cardiovascular diseases, hepatic diseases, angina pectoris, hypertension, heart failure, myocardial infarction, ventricular hypertrophy, vascular diseases, miscrovascular disease, vascular leak syndrome, aneurysm, stroke, embolism, thrombosis, endothelial dysfunction, coronary artery disease, arteriosclerosis, and/or atherosclerosis.

In preferred embodiments, the following N-terminal OATP2 (SNP_ID: PS100s26) deletion polypeptides are encompassed by the present invention: M1-C691, D2-C691, Q3-C691, N4-C691, Q5-C691, H6-C691, L7-C691, N8-C691, K9-C691, T10-C691, A11-C691, E12-C691, A13-C691, Q14-C691, P15-C691, S16-C691, E17-C691, N18-C691, K19-C691, K20-C691, T21-C691, R22-C691, Y23-C691, C24-C691, N25-C691, G26-C691, L27-C691, K28-C691, M29-C691, F30-C691, L31-C691, A32-C691, A33-C691, L34-C691, S35-C691, L36-C691, S37-C691, F38-C691, I39-C691, A40-C691, K41-C691, T42-C691, L43-C691, G44-C691, A45-C691, I46-C691, I47-C691, M48-C691, K49-C691, S50-C691, S51-C691, I52-C691, I53-C691, H54-C691, I55-C691, E56-C691, R57-C691, R58-C691, F59-C691, E60-C691, I61-C691, S62-C691, S63-C691, S64-C691, L65-C691, V66-C691, G67-C691, F68-C691, I69-C691, D70-C691, G71-C691, S72-C691, F73-C691, E74-C691, I75-C691, G76-C691, N77-C691, L78-C691, L79-C691, V80-C691, I81-C691, V82-C691, F83-C691, V84-C691, S85-C691, Y86-C691, F87-C691, G88-C691, S89-C691, K90-C691, L91-C691, H92-C691, R93-C691, P94-C691, K95-C691, L96-C691, I97-C691, G98-C691, I99-C691, G100-C691, C101-C691, F102-C691, I103-C691, M104-C691, G105-C691, I106-C691, G107-C691, G108-C691, V109-C691, L110-C691, T111-C691, A112-C691, L113-C691, P114-C691, H115-C691, F116-C691, F117-C691, M118-C691, G119-C691, Y120-C691, Y121-C691, R122-C691, Y123-C691, S124-C691, K125-C691, E126-C691, T127-C691, N128-C691, I129-C691, D130-C691, S131-C691, S132-C691, E133-C691, N134-C691, S135-C691, T136-C691, S137-C691, T138-C691, L139-C691, S140-C691, T141-C691, C142-C691, L143-C691, I144-C691, N145-C691, Q146-C691, I147-C691, L148-C691, S149-C691, L150-C691, N151-C691, R152-C691, A153-C691, S154-C691, P155-C691, E156-C691, I157-C691, V158-C691, G159-C691, K160-C691, G161-C691, C162-C691, L163-C691, K164-C691, E165-C691, S166-C691, G167-C691, S168-C691, Y169-C691, M170-C691, W171-C691, I172-C691, Y173-C691, V174-C691, F175-C691, M176-C691, G177-C691, N178-C691, M179-C691, L180-C691, R181-C691, G182-C691, I183-C691, G184-C691, E185-C691, T186-C691, P187-C691, I188-C691, V189-C691, P190-C691, L191-C691, G192-C691, L193-C691, S194-C691, Y195-C691, I196-C691, D197-C691, D198-C691, F199-C691, A200-C691, K201-C691, E202-C691, G203-C691, H204-C691, S205-C691, S206-C691, L207-C691, Y208-C691, L209-C691, G210-C691, I211-C691, L212-C691, N213-C691, A214-C691, I215-C691, A216-C691, M217-C691, I218-C691, G219-C691, P220-C691, I221 -C691, I222-C691, G223-C691, F224-C691, T225-C691, L226-C691, G227-C691, S228-C691, L229-C691, F230-C691, S231 -C691, K232-C691, M233-C691, Y234-C691, V235-C691, D236-C691, I237-C691, G238-C691, Y239-C691, V240-C691, D241 -C691, L242-C691, S243-C691, T244-C691, I245-C691, R246-C691, I247-C691, T248-C691, P249-C691, T250-C691, D251 -C691, S252-C691, R253-C691, W254-C691, V255-C691, G256-C691, A257-C691, W258-C691, W259-C691, L260-C691, N261 -C691, F262-C691, L263-C691, V264-C691, S265-C691, G266-C691, L267-C691, F268-C691, S269-C691, 1270-C691, I271-C691, S272-C691, S273-C691, I274-C691, P275-C691, F276-C691, F277-C691, F278-C691, L279-C691, P280-C691, Q281-C691, T282-C691, P283-C691, N284-C691, K285-C691, P286-C691, Q287-C691, K288-C691, E289-C691, R290-C691, K291-C691, A292-C691, S293-C691, L294-C691, S295-C691, L296-C691, H297-C691, V298-C691, L299-C691, E300-C691, T301 -C691, N302-C691, D303-C691, E304-C691, K305-C691, D306-C691, Q307-C691, T308-C691, A309-C691, N310-C691, L311-C691, T312-C691, N313-C691, Q314-C691, G315-C691, K316-C691, N317-C691, I318-C691, T319-C691, K320-C691, N321-C691, V322-C691, T323-C691, G324-C691, F325-C691, F326-C691, Q327-C691, S328-C691, F329-C691, K330-C691, S331-C691, I332-C691, L333-C691, T334-C691, N335-C691, P336-C691, L337-C691, Y338-C691, V339-C691, M340-C691, F341-C691, V342-C691, L343-C691, L344-C691, T345-C691, L346-C691, L347-C691, Q348-C691, V349-C691, S350-C691, S351-C691, Y352-C691, I353-C691, G354-C691, A355-C691, F356-C691, T357-C691, Y358-C691, V359-C691, F360-C691, K361-C691, Y362-C691, V363-C691, E364-C691, Q365-C691, Q366-C691, Y367-C691, G368-C691, Q369-C691, P370-C691, S371-C691, S372-C691, K373-C691, A374-C691, N375-C691, I376-C691, L377-C691, L378-C691, G379-C691, V380-C691, I381-C691, T382-C691, I383-C691, P384-C691, I385-C691, F386-C691, A387-C691, S388-C691, G389-C691, M390-C691, F391-C691, L392-C691, G393-C691, G394-C691, Y395-C691, I396-C691, I397-C691, K398-C691, K399-C691, and/or K400-C691 of SEQ ID NO:16. Polynucleotide sequences encoding these polypeptides are also provided. The present invention also encompasses the use of these N-terminal OATP2 (SNP_ID: PS100s26) deletion polypeptides as immunogenic and/or antigenic epitopes as described elsewhere herein.

In preferred embodiments, the following C-terminal OATP2 (SNP_ID: PS100s26) deletion polypeptides are encompassed by the present invention: M1-C691, M1-H690, M1-T689, M1-E688, M1-S687, M1-D686, M1-A685, M1-G684, M1-A683, M1-S682, M1-P681, M1-V680, M1-F679, M1-H678, M1-K677, M1-N676, M1-K675, M1-N674, M1-L673, M1-S672, M1-E671, M1-L670, M1-N669, M1-A668, M1-E667, M1-D666, M1-M665, M1-V664; M1-S663, M1-G662, M1-N661, M1-E660, M1-S659, M1-A658, M1-N657, M1-I656, M1-D655, M1-K654, M1-E653, M1-Q652, M1-Y651, M1-K650, M1-K649, M1-K648, M1-M647, M1-A646, M1-Y645, M1-I644, M1-L643, M1-I642, M1-I641, M1-Y640, M1-V638, M1-L637, M1-S636, M1-S635, M1-V634, M1-R633, M1-L632, M1-M631, M1-S630, M1-S629, M1-L628, M1-G627, M1-L626, M1-Y625, M1-V624, M1-R623, M1-S622, M1-F621, M1-S620, M1-T619, M1-S618, M1-N617, M1-Y616, M1-T615, M1-R614, M1-C613, M1-S612, M1-G611, M1-R610, M1-T609, M1-G608, M1-C607, M1-N606, M1-N605, M1-T604, M1-S603, M1-W602, M1-K601, M1-I600, M1-C599, M1-T598, M1-T597, M1-D596, M1-I595, M1-L594, M1-A593, M1-G592, M1-F591, M1-Y590, M1-I589, M1-P588, M1-A587, M1-L586, M1-I585, M1-G584, M1-G583, M1-L582, M1-A581, M1-R580, M1-I579, M1-V578, M1-M577, M1-S576, M1-H575, M1-F574, M1-G573, M1-L572, M1-A571, M1-L570, M1-S569, M1-K568, M1-L567, M1-E566, M1-P565, M1-Q564, M1-V563, M1-I562, M1-K561, M1-V560, M1-I559, M1-L558, M1-M557, M1-V556, M1-H555, M1-S554, M1-T553, M1-G552, M1-G551, M1-L550, M1-A549, M1-S548, M1-F547, M1-F546, M1-L545, M1-N544, M1-L543, M1-V542, M1-Q541, M1-I540, M1-A539, M1-V538, M1-F537, M1-F536, M1-Y535, M1-F534, M1-K533, M1-R532, M1-T531, M1-C530, M1-A529, M1-D528, M1-D527, M1-R526, M1-P525, M1-C524, M1-E523, M1-G522, M1-L521, M1-H520, M1-A519, M1-S518, M1-Y517, M1-N516, M1-R515, M1-N514, M1-Q513, M1-L512, M1-G511, M1-T510, M1-V509, M1-E508, M1-L507, M1-C506, M1-S505, M1-C504, M1-N503, M1-Y502, M1-F501, M1-V500, M1-I499, M1-P498, M1-K497, M1-K496, M1-N495, M1-G494, M1-S493, M1-S492, M1-S491, M1-K490, M1-C489, M1-G488, M1-A487, M1-L486, M1-C485, M1-P484, M1-S483, M1-I482, M1-Y481, M1-T480, M1-I479, M1-G478, M1-N477, M1-N476, M1-G475, M1-C474, M1-V473, M1-P472, M1-E471, M1-W470, M1-Q469, M1-S468, M1-E467, M1-D466, M1-C465, M1-N464, M1-C463, M1-D462, M1-S461, M1-N460, M1-C459, M1-Y458, M1-S457, M1-L456, M1-P455, M1-V454, M1-D453, M1-R452, M1-H451, M1-S450, M1-T449, M1-V448, M1-P447, M1-N446, M1-N445, M1-G444, M1-D443, M1-Y442, M1-T441, M1-M440, M1-T439, M1-L438, M1-G437, M1-A436, M1-V435, M1-S434, M1-K433, M1-N432, M1-E431, M1-C430, M1-L429, M1-I428, M1-F427, M1-426, M1-Y425, M1-L424, M1-L423, M1-Y422, M1-F421, M1-S420, M1-L419, M1-S418, M1-M417, M1-V416, M1-A415, M1-T414, M1-F413, M1-C412, M1-S411, M1-F410, M1-K409, M1-A408, M1-I407, M1-G406, M1-V405, M1-T404, M1-N403, M1-L402, M1-K401, and/or M1-K400 of SEQ ID NO:16. Polynucleotide sequences encoding these polypeptides are also provided. The present invention also encompasses the use of these C-terminal OATP2 (SNP_ID: PS100s26) deletion polypeptides as immunogenic and/or antigenic epitopes as described elsewhere herein.

Alternatively, preferred polypeptides of the present invention may comprise polypeptide sequences corresponding to, for example, internal regions of the OATP2 (SNP_ID: PS100s26) polypeptide (e.g., any combination of both N- and C-terminal OATP2 (SNP_ID: PS100s26) polypeptide deletions) of SEQ ID NO:16. For example, internal regions could be defined by the equation: amino acid NX to amino acid CX, wherein NX refers to any N-terminal deletion polypeptide amino acid of OATP2 (SNP_ID: PS100s26) (SEQ ID NO:16), and where CX refers to any C-terminal deletion polypeptide amino acid of OATP2 (SNP_ID: PS100s26) (SEQ ID NO:16). Polynucleotides encoding these polypeptides are also provided. The present invention also encompasses the use of these polypeptides as an immunogenic and/or antigenic epitope as described elsewhere herein. Preferably, the resulting deletion polypeptide comprises the polypeptide polymorphic loci identified elsewhere herein for OATP2 (SNP_ID: PS100s26), and more preferably comprises the polypeptide polymorphic allele identified elsewhere herein for OATP2 (SNP_ID: PS100s26).

### Features of the Polypeptide Encoded by Gene No:7

The present invention relates to isolated nucleic acid molecules comprising, or alternatively, consisting of all or a portion of the variant allele of the human OATP2, solute carrier family 21 member 6 gene (SNP_ID: PS100s29) (e.g., wherein reference or wildtype OATP2 gene is exemplified by SEQ ID NO:1). Preferred portions are at least 10, preferably at least 20, preferably at least 40, preferably at least 100, contiguous polynucleotides and comprise a “C” at the nucleotide position corresponding to nucleotide 655 of the OATP2 gene, or a portion of SEQ ID NO:17. Alternatively, preferred portions are at least 10, preferably at least 20, preferably at least 40, preferably at least 100, contiguous polynucleotides and comprise a “T” at the nucleotide position corresponding to nucleotide 655 of the OATP2 gene, or a portion of SEQ ID NO:17. The invention further relates to isolated gene products, e.g., polypeptides and/or proteins, which are encoded by a nucleic acid molecule comprising all or a portion of the variant allele of the OATP2 gene.

In one embodiment, the invention relates to a method for predicting the likelihood that an individual will have a disorder associated with a “C” at the nucleotide position corresponding to nucleotide position 655 of SEQ ID NO:17 (or diagnosing or aiding in the diagnosis of such a disorder) comprising the steps of obtaining a DNA sample from an individual to be assessed and determining the nucleotide present at position 655 of SEQ ID NO:17. The presence of a “C” at this position indicates that the individual has a greater likelihood of having a disorder associated therewith than an individual having a “T” at that position, or a greater likelihood of having more severe symptoms.

Conversely, the invention relates to a method for predicting the likelihood that an individual will have a disorder associated with a “T” at the nucleotide position corresponding to nucleotide position 655 of SEQ ID NO:17 (or diagnosing or aiding in the diagnosis of such a disorder) comprising the steps of obtaining a DNA sample from an individual to be assessed and determining the nucleotide present at position 655 of SEQ ID NO:17. The presence of a “T” at this position indicates that the individual has a greater likelihood of having a disorder associated therewith than an individual having a “C” at that position, or a greater likelihood of having more severe symptoms.

The present invention further relates to isolated proteins or polypeptides comprising, or alternatively, consisting of all or a portion of the encoded variant amino acid sequence of the human human OATP2, solute carrier family 21 member 6 polypeptide (e.g., wherein reference or wildtype human OATP2, solute carrier family 21 member 6 polypeptide is exemplified by SEQ ID NO:2). Preferred portions are at least 10, preferably at least 20, preferably at least 40, preferably at least 100, contiguous polypeptides and comprises a “T” at the amino acid position corresponding to amino acid 174 of the OATP2 polypeptide, or a portion of SEQ ID NO:18. Alternatively, preferred portions are at least 10, preferably at least 20, preferably at least 40, preferably at least 100, contiguous polypeptides and comprises a “V” at the amino acid position corresponding to amino acid 174 of the OATP2 protein, or a portion of SEQ ID NO:18. The invention further relates to isolated nucleic acid molecules encoding such polypeptides or proteins, as well as to antibodies that bind to such proteins or polypeptides.

Representative disorders which may be detected, diagnosed, identified, treated, prevented, and/or ameliorated by the present invention include, the following, non-limiting diseases and disorders: decreased heptic uptake of administered statins, resistance to beneficial responses of administered statins; resistance to LDL lowering responses to administered statins; resistance to TG lowering responses to administered statins; resistance to HDL elevating responses to administered statins; increased incidence of drug interactions between one or more administered statins; increased incidence of drug and endogenous substance interactions between a statin drug and endogenous substrates of OATP2 including cholate, taurocholate, thyroid hormones T3 and T4, DHEAS, estradiol-17beta-glucuronide, estrone-3-sulfate, prostaglandin E2, thromboxane B2, leukotriene C4, leukotriene E4, and bilirubin and its conjugates; decreased response to HMG-CoA reductase inhibitors; in addition to other disorders referenced herein, which include, for example, any disorder related to high levels of circulating LDL, high levels of TG, and/or low levels of circularing HDL, which include, but are not limited to, cardiovascular diseases, hepatic diseases, angina pectoris, hypertension, heart failure, myocardial infarction, ventricular hypertrophy, vascular diseases, miscrovascular disease, vascular leak syndrome, aneurysm, stroke, embolism, thrombosis, endothelial dysfunction, coronary artery disease, arteriosclerosis, and/or atherosclerosis.

In preferred embodiments, the following N-terminal OATP2 (SNP_ID: PS100s29) deletion polypeptides are encompassed by the present invention: M1-C691, D2-C691, Q3-C691, N4-C691, Q5-C691, H6-C691, L7-C691, N8-C691, K9-C691, T10-C691, A11-C691, E12-C691, A13-C691, Q14-C691, P15-C691, S16-C691, E17-C691, N18-C691, K19-C691, K20-C691, T21-C691, R22-C691, Y23-C691, C24-C691, N25-C691, G26-C691, L27-C691, K28-C691, M29-C691, F30-C691, L31-C691, A32-C691, A33-C691, L34-C691, S35-C691, L36-C691, S37-C691, F38-C691, I39-C691, A40-C691, K41-C691, T42-C691, L43-C691, G44-C691, A45-C691, I46-C691, I47-C691, M48-C691, K49-C691, S50-C691, S51-C691, I52-C691, I53-C691, H54-C691, I55-C691, E56-C691, R57-C691, R58-C691, F59-C691, E60-C691, I61-C691, S62-C691, S63-C691, S64-C691, L65-C691, V66-C691, G67-C691, F68-C691, I69-C691, D70-C691, G71-C691, S72-C691, F73-C691, E74-C691, I75-C691, G76-C691, N77-C691, L78-C691, L79-C691, V80-C691, I81-C691, V82-C691, F83-C691, V84-C691, S85-C691, Y86-C691, F87-C691, G88-C691, S89-C691, K90-C691, L91-C691, H92-C691, R93-C691, P94-C691, K95-C691, K95-C691, L96-C691, I97-C691, G98-C691, I99-C691, G100-C691, C101-C691, F102-C691, I103-C691, M104-C691, G105-C691, I106-C691, G107-C691, G108-C691, V109-C691, L110-C691, T111-C691, A112-C691, L113-C691, P114-C691, H115-C691, F116-C691, F117-C691, M118-C691, G119-C691, Y120-C691, Y121-C691, R122-C691, Y123-C691, S124-C691, K125-C691, E126-C691, T127-C691, N128-C691, I129-C691, D130-C691, S131-C691, S132-C691, E133-C691, N134-C691, S135-C691, T136-C691, S137-C691, T138-C691, L139-C691, S140-C691, T141-C691, C142-C691, L143-C691, I144-C691, N145-C691, Q146-C691, I147-C691, L148-C691, S149-C691, L150-C691, N151-C691, R152-C691, A153-C691, S154-C691, P155-C691, E156-C691, I157-C691, V158-C691, G159-C691, K160-C691, G161-C691, C162-C691, L163-C691, K164-C691, E165-C691, S166-C691, G167-C691, S168-C691, Y169-C691, M170-C691, W171-C691, I172-C691, Y173-C691, and/or A174-C691 of SEQ ID NO:18. Polynucleotide sequences encoding these polypeptides are also provided. The present invention also encompasses the use of these N-terminal OATP2 (SNP_ID: PS100s29) deletion polypeptides as immunogenic and/or antigenic epitopes as described elsewhere herein.

In preferred embodiments, the following C-terminal OATP2 (SNP_ID: PS100s29) deletion polypeptides are encompassed by the present invention: M1-C691, M1-H690, M1-T689, M1-E688, M1-S687, M1-D686, M1-A685, M1-G684, M1-A683, M1-S682, M1-P681, M1-V680, M1-F679, M1-H678, M1-K677, M1-N676, M1-K675, M1-N674, M1-L673, M1-S672, M1-E671, M1-L670, M1-N669, M1-A668, M1-E667, M1-D666, M1-M665, M1-V664, M1-S663, M1-G662, M1-N661, M1-E660, M1-S659, M1-A658, M1-N657, M1-I656, M1-D655, M1-K654, M1-E653, M1-Q652, M1-Y651, M1-K650, M1-K649, M1-K648, M1-M647, M1-A646, M1-Y645, M1-I644, M1-L643, M1-I642, M1-I641, M1-Y640, M1-L639, M1-V638, M1-L637, M1-S636, M1-S635, M1-V634, M1-R633, M1-L632, M1-M631, M1-S630, M1-S629, M1-L628, M1-G627, M1-L626, M1-Y625, M1-V624, M1-R623, M1-S622, M1-F621, M1-S620, M1-T619, M1-S618, M1-N617, M1-Y616, M1-T615, M1-R614, M1-C613, M1-S612, M1-G611, M1-R610, M1-T609, M1-G608, M1-C607, M1-N606, M1-N605, M1-T604, M1-S603, M1-W602, M1-K601, M1-I600, M1-C599, M1-T598, M1-T597, M1-D596, M1-I595, M1-L594, M1-A593, M1-G592, M1-F591, M1-Y590, M1-I589, M1-P588, M1-A587, M1-L586, M1-I585, M1-G584, M1-G583, M1-L582, M1-A581, M1-R580, M1-I579, M1-V578, M1-M577, M1-S576, M1-H575, M1-F574, M1-G573, M1-L572, M1-A571, M1-L570, M1-S569, M1-K568, M1-L567, M1-E566, M1-P565, M1-Q564, M1-V563, M1-I562, M1-K561, M1-V560, M1-I559, M1-L558; M1-M557, M1-V556, M1-H555, M1-S554, M1-T553, M1-G552, M1-G551, M1-L550, M1-A549, M1-S548, M1-F547, M1-F546, M1-L545, M1-N544, M1-L543, M1-V542, M1-Q541, M1-I540, M1-A539, M1-V538, M1-F537, M1-F536, M1-Y535, M1-F534, M1-K533, M1-R532, M1-T531, M1-C530, M1-A529, M1-D528, M1-D527, M1-R526, M1-P525, M1-C524, M1-E523, M1-G522, M1-L521, M1-H520, M1-A519, M1-S518, M1-Y517, M1-N516, M1-R515, M1-N514, M1-Q513, M1-L512, M1-G511, M1-T510, M1-V509, M1-E508, M1-L507, M1-C506, M1-S505, M1-C504, M1-N503, M1-Y502, M1-F501, M1-V500, M1-I499, M1-P498, M1-K497, M1-K496, M1-N495, M1-G494, M1-S493, M1-S492, M1-S491, M1-K490, M1-C489, M1-G488, M1-A487, M1-L486, M1-C485, M1-P484, M1-S483, M1-I482, M1-Y481, M1-T480, M1-I479, M1-G478, M1-N477, M1-N476, M1-G475, M1-C474, M1-V473, M1-P472, M1-E471, M1-W470, M1-Q469, M1-S468, M1-E467, M1-D466, M1-C465, M1-N464, M1-C463, M1-D462, M1-S461, M1-N460, M1-C459, M1-Y458, M1-S457, M1-L456, M1-P455, M1-V454, M1-D453, M1-R452, M1-H451, M1-S450, M1-T449, M1-V448, M1-P447, M1-N446, M1-N445, M1-G444, M1-D443, M1-Y442, M1-T441, M1-M440, M1-T439, M1-L438, M1-G437, M1-A436, M1-V435, M1-S434, M1-K433, M1-N432, M1-E431, M1-C430, M1-L429, M1-I428, M1-F427, M1-F426, M1-Y425, M1-L424, M1-L423, M1-Y422, M1-F421, M1-S420, M1-L419, M1-S418, M1-M417, M1-V416, M1-A415, M1-T414, M1-F413, M1-C412, M1-S411, M1-F410, M1-K409, M1-A408, M1-I407, M1-G406, M1-V405, M1-T404, M1-N403, M1-L402, M1-K401, M1-F400, M1-K399, M1-K398, M1-I397, M1-I396, M1-Y395, M1-G394, M1-G393, M1-L392, M1-F391, M1-M390, M1-G389, M1-S388, M1-A387, M1-F386, M1-I385, M1-P384, M1-I383, M1-T382, M1-I381,, M1-V380, M1-G379, M1-L378, M1-L377, M1-I376, M1-N375, M1-A374, M1-K373, M1-S372, M1-S371, M1-P370, M1-Q369, M1-G368, M1-Y367, M1-Q366, M1-Q365, M1-E364, M1-V363, M1-Y362, M1-K361, M1-F360, M1-V359, M1-Y358, M1-T357, M1-F356, M1-A355, M1-G354, M1-I353, M1-Y352, M1-S351, M1-S350, M1-V349, M1-Q348, M1-L347, M1-L346, M1-T345, M1-L344, M1-L343, M1-V342, M1-F341, M1-M340, M1-V339, M1-Y338, M1-L337, M1-P336, M1-N335, M1-T334, M1-L333, M1-I332, M1-S331, M1-K330, M1-F329, M1-S328, M1-Q327, M1-F326, M1-F325, M1-G324, M1-T323, M1-V322, M1-N321, M1-K320, M1-T319, M1-I318, M1-N317, M1-K316, M1-G315, M1-Q314, M1-N313, M1-T312, M1-L311, M1-N310, M1-A309, M1-T308, M1-Q307, M1-D306, M1-K305, M1-E304, M1-D303, M1-N302, M1-T301, M1-E300, M1-L299, M1-V298, M1-H297, M1-L296, M1-S295, M1-L294, M1-S293, M1-A292, M1-K291, M1-R290, M1-E289, M1-K288, M1-Q287, M1-P286, M1-K285, M1-N284, M1-P283, M1-T282, M1-Q281, M1-P280, M1-L279, M1-F278, M1-F277, M1-F276, M1-P275, M1-I274, M1-S273, M1-S272, M1-I271, M1-I270, M1-S269, M1-F268, M1-L267, M1-G266, M1-S265, M1-V264, M1-L263, M1-F262, M1-N261, M1-L260, M1-W259, M1-W258, M1-A257, M1-G256, M1-V255, M1-W254, M1-R253, M1-S252, M1-D251, M1-T250, M1-P249, M1-T248, M1-I247, M1-R246, M1-I245, M1-T244, M1-S243, M1-L242, M1-D241, M1-V240, M1-Y239, M1-G238, M1-I237, M1-D236, M1-V235, M1-Y234, M1-M233, M1-K232, M1-S231, M1-F230, M1-L229, M1-S228, M1-G227, M1-L226, M1-T225, M1-F224, M1-G223, M1-I222, M1-I221, M1-P220, M1-G219, M1-I218, M1-M217, M1-A216, M1-I215, M1-A214, M1-N213, M1-L212, M1-I211, M1-G210, M1-L209, M1-Y208, M1-L207, M1-S206, M1-S205, M1-H204, M1-G203, M1-E202, M1-K201, M1-A200, M1-F199, M1-D198, M1-D197, M1-I196, M1-Y195, M1-S194, M1-L193, M1-G192, M1-L191, M1-P190, M1-V189, M1-I188, M1-P187, M1-T186, M1-E185, M1-G184, M1-I183, M1-G182, M1-R181, M1-L180, M1-M179, M1-N178, M1-G177, M1-M176, M1-F175, and/or M1-A174 of SEQ ID NO:18. Polynucleotide sequences encoding these polypeptides are also provided. The present invention also encompasses the use of these C-terminal OATP2 (SNP_ID: PS100s29) deletion polypeptides as immunogenic and/or antigenic epitopes as described elsewhere herein.

Alternatively, preferred polypeptides of the present invention may comprise polypeptide sequences corresponding to, for example, internal regions of the OATP2 (SNP_ID: PS100s29) polypeptide (e.g., any combination of both N- and C-terminal OATP2 (SNP_ID: PS100s29) polypeptide deletions) of SEQ ID NO:18. For example, internal regions could be defined by the equation: amino acid NX to amino acid CX, wherein NX refers to any N-terminal deletion polypeptide amino acid of OATP2 (SNP_ID: PS100s29) (SEQ ID NO:18), and where CX refers to any C-terminal deletion polypeptide amino acid of OATP2 (SNP_ID: PS100s29) (SEQ ID NO:18). Polynucleotides encoding these polypeptides are also provided. The present invention also encompasses the use of these polypeptides as an immunogenic and/or antigenic epitope as described elsewhere herein. Preferably, the resulting deletion polypeptide comprises the polypeptide polymorphic loci identified elsewhere herein for OATP2 (SNP_ID: PS100s29), and more preferably comprises the polypeptide polymorphic allele identified elsewhere herein for OATP2 (SNP_ID: PS100s29).

### Features of the Polypeptide Encoded by Gene No: 8

The present invention relates to isolated nucleic acid molecules comprising, or alternatively, consisting of all or a portion of the variant allele of the human OATP2, solute carrier family 21 member 6 gene (SNP_ID: PS100s30) (e.g., wherein reference or wildtype OATP2 gene is exemplified by SEQ ID NO:1). Preferred portions are at least 10, preferably at least 20, preferably at least 40, preferably at least 100, contiguous polynucleotides and comprise a “C” at the nucleotide position corresponding to nucleotide 705 of the OATP2 gene, or a portion of SEQ ID NO:19. Alternatively, preferred portions are at least 10, preferably at least 20, preferably at least 40, preferably at least 100, contiguous polynucleotides and comprise a “T” at the nucleotide position corresponding to nucleotide 705 of the OATP2 gene, or a portion of SEQ ID NO:19. The invention further relates to isolated gene products, e.g., polypeptides and/or proteins, which are encoded by a nucleic acid molecule comprising all or a portion of the variant allele of the OATP2 gene.

In one embodiment, the invention relates to a method for predicting the likelihood that an individual will have a disorder associated with a “C” at the nucleotide position corresponding to nucleotide position 705 of SEQ ID NO:19 (or diagnosing or aiding in the diagnosis of such a disorder) comprising the steps of obtaining a DNA sample from an individual to be assessed and determining the nucleotide present at position 705 of SEQ ID NO:19. The presence of a “C” at this position indicates that the individual has a greater likelihood of having a disorder associated therewith than an individual having a “T” at that position, or a greater likelihood of having more severe symptoms.

Conversely, the invention relates to a method for predicting the likelihood that an individual will have a disorder associated with a “T” at the nucleotide position corresponding to nucleotide position 705 of SEQ ID NO:19 (or diagnosing or aiding in the diagnosis of such a disorder) comprising the steps of obtaining a DNA sample from an individual to be assessed and determining the nucleotide present at position 705 of SEQ ID NO:19. The presence of a “T” at this position indicates that the individual has a greater likelihood of having a disorder associated therewith than an individual having a “C” at that position, or a greater likelihood of having more severe symptoms.

The present invention further relates to isolated proteins or polypeptides comprising, or alternatively, consisting of all or a portion of the encoded variant amino acid sequence of the human human OATP2, solute carrier family 21 member 6 polypeptide (e.g., wherein reference or wildtype human OATP2, solute carrier family 21 member 6 polypeptide is exemplified by SEQ ID NO:2). Preferred portions are at least 10, preferably at least 20, preferably at least 40, preferably at least 100, contiguous polypeptides and comprises a “K” at the amino acid position corresponding to amino acid 191 of the OATP2 polypeptide, or a portion of SEQ ID NO:20. Alternatively, preferred portions are at least 10, preferably at least 20, preferably at least 40, preferably at least 100, contiguous polypeptides and comprises a “L” at the amino acid position corresponding to amino acid 191 of the OATP2 protein, or a portion of SEQ ID NO:20. The invention further relates to isolated nucleic acid molecules encoding such polypeptides or proteins, as well as to antibodies that bind to such proteins or polypeptides.

Representative disorders which may be detected, diagnosed, identified, treated, prevented, and/or ameliorated by the present invention include, the following, non-limiting diseases and disorders: decreased heptic uptake of administered statins, resistance to beneficial responses of administered statins; resistance to LDL lowering responses to administered statins; resistance to TG lowering responses to administered statins; resistance to HDL elevating responses to administered statins; increased incidence of drug interactions between one or more administered statins; increased incidence of drug and endogenous substance interactions between a statin drug and endogenous substrates of OATP2 including cholate, taurocholate, thyroid hormones T3 and T4, DHEAS, estradiol-17beta-glucuronide, estrone-3-sulfate, prostaglandin E2, thromboxane B2, leukotriene C4, leukotriene E4, and bilirubin and its conjugates; decreased response to HMG-CoA reductase inhibitors; in addition to other disorders referenced herein, which include, for example, any disorder related to high levels of circulating LDL, high levels of TG, and/or low levels of circularing HDL, which include, but are not limited to, cardiovascular diseases, hepatic diseases, angina pectoris, hypertension, heart failure, myocardial infarction, ventricular hypertrophy, vascular diseases, miscrovascular disease, vascular leak syndrome, aneurysm, stroke, embolism, thrombosis, endothelial dysfunction, coronary artery disease, arteriosclerosis, and/or atherosclerosis.

In preferred embodiments, the following N-terminal OATP2 (SNP-ID: PS100s30) deletion polypeptides are encompassed by the present invention: M1-C691, D2-C691, Q3-C691, N4-C691, Q5-C691, H6-C691, L7-C691, N8-C691, K9-C691, T10-C691, A11-C691, E12-C691, A13-C691, Q14-C691, P15-C691, S16-C691, E17-C691, N18-C691, K19-C691, K20-C691, T21-C691, R22-C691, Y23-C691, C24-C691, N25-C691, G26-C691, L27-C691, K28-C691, M29-C691, F30-C691, L31-C691, A32-C691, A33-C691, L34-C691, S35-C691, L36-C691, S37-C691, F38-C691, I39-C691, A40-C691, K41-C691, T42-C691, L43-C691, G44-C691, A45-C691, I46-C691, I47-C691, M48-C691, K49-C691, S50-C691, S51-C691, I52-C691, I53-C691, H54-C691, I55-C691, E56-C691, R57-C691, R58-C691, F59-C691, E60-C691, I61-C691, S62-C691, S63-C691, S64-C691, L65-C691, V691, G67-C691, F68-C691, I69-C691, D70-C691, G71-C691, S72-C691, F73-C691, E74-C691, I75-C691, G76-C691, N77-C691, L78-C691, L79-C691, V80-C691, I81-C691, V82-C691, F83-C691, V84-C691, S85-C691, Y86-C691, F87-C691, G88-C691, S89-C691, K90-C691, L91-C691, H92-C691, R93-C691, P94-C691, K95-C691, L96-C691, I97-C691, G98-C691, I99-C691, G100-C691, C101-C691, F102-C691, I103-C691, M104-C691, G105-C691, I106-C691, G107-C691, G108-C691, V109-C691, L110-C691, T111-C691, A112-C691, L113-C691, P114-C691, H115-C691, F116-C691, F117-C691, M118-C691, G119-C691, Y120-C691, Y121-C691, R122-C691, Y123-C691, S124-C691, K125-C691, E126-C691, T127-C691, N128-C691, I129-C691, D130-C691, S131-C691, S132-C691, E133-C691, N134-C691, S135-C691, T136-C691, S137-C691, T138-C691, L139-C691, S140-C691, T141-C691, C142-C691, L143-C691, I144-C691, N145-C691, Q146-C691, I147-C691, L148-C691, S149-C691, L150-C691, N151-C691, R152-C691, A153-C691, S154-C691, P155-C691, E156-C691, I157-C691, V158-C691, G159-C691, K160-C691, G161-C691, C162-C691, L163-C691, K164-C691, E165-C691, S166-C691, G167-C691, S168-C691, Y169-C691, M170-C691, W171-C691, I172-C691, Y173-C691, V174-C691, F175-C691, M176-C691, G177-C691, N178-C691, M179-C691, L180-C691, R181-C691, G182-C691, I183-C691, G184-C691, E185-C691, T186-C691, P187-C691, I188-C691, V189-C691, P190-C691, and/or L191-C691 of SEQ ID NO:20. Polynucleotide sequences encoding these polypeptides are also provided. The present invention also encompasses the use of these N-terminal OATP2 (SNP-ID: PS100s30) deletion polypeptides as immunogenic and/or antigenic epitopes as described elsewhere herein.

In preferred embodiments, the following C-terminal OATP2 (SNP-ID: PS100s30) deletion polypeptides are encompassed by the present invention: M1-C691, M1-H690, M1-T689, M1-E688, M1-S687, M1-D686, M1-A685, M1-G684, M1-A683, M1-S682, M1-P681, M1-V680, M1-F679, M1-H678, M1-K677, M1-N676, M1-K675, M1-N674, M1-L673, M1-S672, M1-E671, M1-L670, M1-N669, M1-A668, M1-E667, M1-D666, M1-M665, M1-V664, M1-S663, M1-G662, M1-N661, M1-E660, M1-S659, M1-A658, M1-N657, M1-I656, M1-D655, M1-K654, M1-E653, M1-Q652, M1-Y651, M1-K650, M1-K649, M1-K648, M1-M647, M1-A646, M1-Y645, M1-I644, M1-L643, M1-I642, M1-I641, M1-Y640, M1-L639, M1-V638, M1-L637, M1-S636, M1-S635, M1-V634, M1-R633, M1-L632, M1-M631, M1-S630, M1-S629, M1-L628, M1-G627, M1-L626, M1-Y625, M1-V624, M1-R623, M1-S622, M1-F621, M1-S620, M1-T619, M1-S618, M1-N617, M1-Y616, M1-T615, M1-R614, M1-C613, M1-S612, M1-G611, M1-R610, M1-T609, M1-G608, M1-C607, M1-N606, M1-N605, M1-T604, M1-S603, M1-W602, M1-K601, M1-I600, M1-C599, M1-T598, M1-T597, M1-D596, M1-I595, M1-L594, M1-A593, M1-G592, M1-F591, M1-Y590, M1-I589, M1-P588, M1-A587, M1-L586, M1-I585, M1-G584, M1-G583, M1-L582, M1-A581, M1-R580, M1-I579, M1-V578, M1-M577, M1-S576, M1-H575, M1-F574, M1-G573, M1-L572, M1-A571, M1-L570, M1-S569, M1-K568, M1-L567, M1-E566, M1-P565, M1-Q564, M1-V563, M1-I562, M1-K561, M1-V560, M1-I559, M1-L558, M1-M557, M1-V556, M1-H555, M1-S554, M1-T553, M1-G552, M1-G551, M1-L550, M1-A549, M1-S548, M1-F547, M1-F546, M1-L545, M1-N544, M1-L543, M1-V542, M1-Q541, M1-I540, M1-A539, M1-V538, M1-F537, M1-F536, M1-Y535, M1-F534, M1-K533, M1-R532, M1-T531, M1-C530, M1-A529, M1-D528, M1-D527, M1-R526, M1-P525, M1-C524, M1-E523, M1-G522, M1-L521, M1-H520, M1-A519, M1-S518, M1-Y517, M1-N516, M1-R515, M1-N514, M1-Q513, M1-L512, M1-G511, M1-T510, M1-V509, M1-E508, M1-L507, M1-C506, M1-S505, M1-C504, M1-N503, M1-Y502, M1-F501, M1-V500, M1-I499, M1-P498, M1-K497, M1-K496, M1-N495, M1-G494, M1-S493, M1-S492, M1-S491, M1-K490, M1-C489, M1-G488, M1-A487, M1-L486, M1-C485, M1-P484, M1-S483, M1-I482, M1-Y481, M1-T480, M1-I479, M1-G478, M1-N477, M1-N476, M1-G475, M1-C474, M1-V473, M1-P472, M1-E471, M1-W470, M1-Q469, M1-S468, M1-E467, M1-D466, M1-C465, M1-N464, M1-C463, M1-D462, M1-S461, M1-N460, M1-C459, M1-Y458, M1-S457, M1-L456, M1-P455, M1-V454, M1-D453, M1-R452, M1-H451, M1-S450, M1-T449, M1-V448, M1-P447, M1-N446, M1-N445, M1-G444, M1-D443, M1-Y442, M1-T441, M1-M440, M1-T439, M1-L438, M1-G437, M1-A436, M1-V435, M1-S434, M1-K433, M1-N432, M1-E431, M1-C430, M1-L429, M1-I428, M1-F427, M1-F426, M1-Y425, M1-L424, M1-L423, M1-Y422, M1-F421, M1-S420, M1-L419, M1-S418, M1-M417, M1-V416, M1-A415, M1-T414, M1-F413, M1-C412, M1-S411, M1-F410, M1-K409, M1-A408, M1-I407, M1-G406, M1-V405, M1-T404, M1-N403, M1-L402, M1-K401, M1-F400, M1-K399, M1-K398, M1-I397, M1-I396, M1-Y395, M1-G394, M1-G393, M1-L392, M1-F391, M1-M390, M1-G389, M1-S388, M1-A387, M1-F386, M1-I385, M1-P384, M1-I383, M1-T382, M1-I381, M1-V380, M1-G379, M1-L378, M1-L377, M1-I376, M1-N375, M1-A374, M1-K373, M1-S372, M1-S371, M1-P370, M1-Q369, M1-G368, M1-Y367, M1-Q366, M1-Q365, M1-E364, M1-V363, M1-Y362, M1-K361, M1-F360, M1-V359, M1-Y358, M1-T357, M1-F356, M1-A355, M1-G354, M1-I353, M1-Y352, M1-S351, M1-S350, M1-V349, M1-Q348, M1-L347, M1-L346, M1-T345, M1-L344, M1-L343, M1-V342, M1-F341, M1-M340, M1-V339, M1-Y338, M1-L337, M1-P336, M1-N335, M1-T334, M1-L333, M1-I332, M1-S331, M1-K330, M1-F329, M1-S328, M1-Q327, M1-F326, M1-F325, M1-G324, M1-T323, M1-V322, M1-N321, M1-K320, M1-T319, M1-I318, M1-N317, M1-K316, M1-G315, M1-Q314, M1-N313, M1-T312, M1-L311, M1-N310, M1-A309, M1-T308, M1-Q307, M1-D306, M1-K305, M1-E304, M1-D303, M1-N302, M1-T301, M1-E300, M1-L299, M1-V298, M1-H297, M1-L296, M1-S295, M1-L294, M1-S293, M1-A292, M1-K291, M1-R290, M1-E289, M1-K288, M1-Q287, M1-P286, M1-K285, M1-N284, M1-P283, M1-T282, M1-Q281, M1-P280, M1-L279, M1-F278, M1-F277, M1-F276, M1-P275, M1-I274, M1-S273, M-1-S272, M1-I271, M1-I270, M1-S269, M1-F268, M1-L267, M1-G266, M1-S265, M1-V264, M1-L263, M1-F262, M1-N261, M1-L260, M1-W259, M1-W258, M1-A257, M1-G256, M1-V255, M1-W254, M1-R253, M1-S252, M1-D251, M1-T250, M1-P249, M1-T248, M1-I247, M1-R246, M1-I245, M1-T244, M1-S243, M1-L242, M1-D241, M1-V240, M1-Y239, M1-G238, M1-I237, M1-D236, M1-V235, M1-Y234, M1-M233, M1-K232, M1-S231, M1-F230, M1-L229, M1-S228, M1-G227, M1-L226, M1-T225, M1-F224, M1 -G223, M1-I222, M1-I221, M1-P220, M1-G219, M1-I218, M1-M217, M1-A216, M1-I215, M1-A214, M1-N213, M1-L212, M1-I211, M-1-G210, M1-L209, M1-Y208, M1-L207, M1-S206, M1-S205, M1-H204, M1-G203, M1-E202, M1-K201, M1-A200, M1-F199, M1-D198, M1-D197, M1-I196, M1-Y195, M1-S194, M1-L193, M1-G192, and/or M1-L191 of SEQ ID NO:20. Polynucleotide sequences encoding these polypeptides are also provided. The present invention also encompasses the use of these C-terminal OATP2 (SNP-ID: PS100s30) deletion polypeptides as immunogenic and/or antigenic epitopes as described elsewhere herein.

Alternatively, preferred polypeptides of the present invention may comprise polypeptide sequences corresponding to, for example, internal regions of the OATP2 (SNP_ID: PS100s30) polypeptide (e.g., any combination of both N- and C-terminal OATP2 (SNP_ID: PS100s30) polypeptide deletions) of SEQ ID NO:20. For example, internal regions could be defined by the equation: amino acid NX to amino acid CX, wherein NX refers to any N-terminal deletion polypeptide amino acid of OATP2 (SNP_ID: PS100s30) (SEQ ID NO:20), and where CX refers to any C-terminal deletion polypeptide amino acid of OATP2 (SNP_ID: PS100s30) (SEQ ID NO:20). Polynucleotides encoding these polypeptides are also provided. The present invention also encompasses the use of these polypeptides as an immunogenic and/or antigenic epitope as described elsewhere herein. Preferably, the resulting deletion polypeptide comprises the polypeptide polymorphic loci identified elsewhere herein for OATP2 (SNP_ID: PS100s30), and more preferably comprises the polypeptide polymorphic allele identified elsewhere herein for OATP2 (SNP_ID: PS100s30).

### Features of the Polypeptide Encoded by Gene No: 9

The present invention relates to isolated nucleic acid molecules comprising, or alternatively, consisting of all or a portion of the variant allele of the human OATP2, solute carrier family 21 member 6 gene (SNP_ID: PS100s31) (e.g., wherein reference or wildtype OATP2 gene is exemplified by SEQ ID NO:1). Preferred portions are at least 10, preferably at least 20, preferably at least 40, preferably at least 100, contiguous polynucleotides and comprise a “T at the nucleotide position corresponding to nucleotide 731 of the OATP2 gene, or a portion of SEQ ID NO:21. Alternatively, preferred portions are at least 10, preferably at least 20, preferably at least 40, preferably at least 100, contiguous polynucleotides and comprise a “C” at the nucleotide position corresponding to nucleotide 731 of the OATP2 gene, or a portion of SEQ ID NO:21. The invention further relates to isolated gene products, e.g., polypeptides and/or proteins, which are encoded by a nucleic acid molecule comprising all or a portion of the variant allele of the OATP2 gene.

In one embodiment, the invention relates to a method for predicting the likelihood that an individual will have a disorder associated with a “T” at the nucleotide position corresponding to nucleotide position 731 of SEQ ID NO:21 (or diagnosing or aiding in the diagnosis of such a disorder) comprising the steps of obtaining a DNA sample from an individual to be assessed and determining the nucleotide present at position 731 of SEQ ID NO:21. The presence of a “T” at this position indicates that the individual has a greater likelihood of having a disorder associated therewith than an individual having a “C” at that position, or a greater likelihood of having more severe symptoms.

Conversely, the invention relates to a method for predicting the likelihood that an individual will have a disorder associated with a “C” at the nucleotide position corresponding to nucleotide position 731 of SEQ ID NO:21 (or diagnosing or aiding in the diagnosis of such a disorder) comprising the steps of obtaining a DNA sample from an individual to be assessed and determining the nucleotide present at position 731 of SEQ ID NO:21. The presence of a “C” at this position indicates that the individual has a greater likelihood of having a disorder associated therewith than an individual having a “T” at that position, or a greater likelihood of having more severe symptoms.

Representative disorders which may be detected, diagnosed, identified, treated, prevented, and/or ameliorated by the present invention include, the following, non-limiting diseases and disorders: decreased heptic uptake of administered statins, resistance to beneficial responses of administered statins; resistance to LDL lowering responses to administered statins; resistance to TG lowering responses to administered statins; resistance to HDL elevating responses to administered statins; increased incidence of drug interactions between one or more administered statins; increased incidence of drug and endogenous substance interactions between a statin drug and endogenous substrates of OATP2 including cholate, taurocholate, thyroid hormones T3 and T4, DHEAS, estradiol-17beta-glucuronide, estrone-3-sulfate, prostaglandin E2, thromboxane B2, leukotriene C4, leukotriene E4, and bilirubin and its conjugates; decreased response to HMG-CoA reductase inhibitors; in addition to other disorders referenced herein, which include, for example, any disorder related to high levels of circulating LDL, high levels of TG, and/or low levels of circularing HDL, which include, but are not limited to, cardiovascular diseases, hepatic diseases, angina pectoris, hypertension, heart failure, myocardial infarction, ventricular hypertrophy, vascular diseases, miscrovascular disease, vascular leak syndrome, aneurysm, stroke, embolism, thrombosis, endothelial dysfunction, coronary artery disease, arteriosclerosis, and/or atherosclerosis.

### Features of the Polypeptide Encoded by Gene No: 10

The present invention relates to isolated nucleic acid molecules comprising, or alternatively, consisting of all or a portion of the variant allele of the human cMOAT, ATP-binding cassette sub-family C member 2 gene (SNP_ID: PS101s1) (e.g., wherein reference or wildtype cMOAT gene is exemplified by SEQ ID NO:1). Preferred portions are at least 10, preferably at least 20, preferably at least 40, preferably at least 100, contiguous polynucleotides and comprise a “G” at the nucleotide position corresponding to nucleotide 3664 of the cMOAT gene, or a portion of SEQ ID NO:23. Alternatively, preferred portions are at least 10, preferably at least 20, preferably at least 40, preferably at least 100, contiguous polynucleotides and comprise a “T” at the nucleotide position corresponding to nucleotide 3664 of the cMOAT gene, or a portion of SEQ ID NO:23. The invention further relates to isolated gene products, e.g., polypeptides and/or proteins, which are encoded by a nucleic acid molecule comprising all or a portion of the variant allele of the cMOAT gene.

In one embodiment, the invention relates to a method for predicting the likelihood that an individual will have a disorder associated with a “G” at the nucleotide position corresponding to nucleotide position 3664 of SEQ ID NO:23 (or diagnosing or aiding in the diagnosis of such a disorder) comprising the steps of obtaining a DNA sample from an individual to be assessed and determining the nucleotide present at position 3664 of SEQ ID NO:23. The presence of a “G” at this position indicates that the individual has a greater likelihood of having a disorder associated therewith than an individual having a “T” at that position, or a greater likelihood of having more severe symptoms.

Conversely, the invention relates to a method for predicting the likelihood that an individual will have a disorder associated with a “T” at the nucleotide position corresponding to nucleotide position 3664 of SEQ ID NO:23 (or diagnosing or aiding in the diagnosis of such a disorder) comprising the steps of obtaining a DNA sample from an individual to be assessed and determining the nucleotide present at position 3664 of SEQ ID NO:23. The presence of a “T” at this position indicates that the individual has a greater likelihood of having a disorder associated therewith than an individual having a “G” at that position, or a greater likelihood of having more severe symptoms.

The present invention further relates to isolated proteins or polypeptides comprising, or alternatively, consisting of all or a portion of the encoded variant amino acid sequence of the human human cMOAT, solute carrier family 21 member 6 polypeptide (e.g., wherein reference or wildtype human cMOAT, solute carrier family 21 member 6 polypeptide is exemplified by SEQ ID NO:2). Preferred portions are at least 10, preferably at least 20, preferably at least 40, preferably at least 100, contiguous polypeptides and comprises a “V” at the amino acid position corresponding to amino acid 1188 of the cMOAT polypeptide, or a portion of SEQ ID NO:24. Alternatively, preferred portions are at least 10, preferably at least 20, preferably at least 40, preferably at least 100, contiguous polypeptides and comprises a “E” at the amino acid position corresponding to amino acid 1188 of the cMOAT protein, or a portion of SEQ ID NO:24. The invention further relates to isolated nucleic acid molecules encoding such polypeptides or proteins, as well as to antibodies that bind to such proteins or polypeptides.

Representative disorders which may be detected, diagnosed, identified, treated, prevented, and/or ameliorated by the present invention include, the following, non-limiting diseases and disorders: decreased heptic uptake of administered statins, resistance to beneficial responses of administered statins; resistance to LDL lowering responses to administered statins; resistance to TG lowering responses to administered statins; resistance to HDL elevating responses to administered statins; increased incidence of drug interactions between one or more administered statins; increased incidence of drug and endogenous substance interactions between a statin drug and endogenous substrates of cMOAT including cholate, taurocholate, thyroid hormones T3 and T4, DHEAS, estradiol-17beta-glucuronide, estrone-3-sulfate, prostaglandin E2, thromboxane B2, leukotriene C4, leukotriene E4, and bilirubin and its conjugates; decreased response to HMG-CoA reductase inhibitors; in addition to other disorders referenced herein, which include, for example, any disorder related to high levels of circulating LDL, high levels of TG, and/or low levels of circularing HDL, which include, but are not limited to, cardiovascular diseases, hepatic diseases, angina pectoris, hypertension, heart failure, myocardial infarction, ventricular hypertrophy, vascular diseases, miscrovascular disease, vascular leak syndrome, aneurysm, stroke, embolism, thrombosis, endothelial dysfunction, coronary artery disease, arteriosclerosis, and/or atherosclerosis.

In preferred embodiments, the following N-terminal cMOAT (SNP_ID: PS101s1) deletion polypeptides are encompassed by the present invention: M1-F1545, L2-F1545, E3-F1545, K4-F1545, F5-F1545, C6-F1545, N7-F1545, S8-F1545, T9-F1545, F10-F1545, W11-F1545, N12-F1545, S13-F1545, S14-F1545, F15-F1545, L16-F1545, D17-F1545, S18-F1545, P19-F1545, E20-F1545, A21-F1545, D22-F1545, L23-F1545, P24-F1545, L25-F1545, C26-F1545, F27-F1545, E28-F1545, Q29-F1545, T30-F1545, V31-F1545, L32-F1545, V33-F1545, W34-F1545, I35-F1545, P36-F1545, L37-F1545, G38-F1545, F39-F1545, L40-F1545, W41-F1545, L42-F1545, L43-F1545, A44-F1545, P45-F1545, W46-F1545, Q47-F1545, L48-F1545, L49-F1545, H50-F1545, V51-F1545, Y52-F1545, K53-F1545, S54-F1545, R55-F1545, T56-F1545, K57-F1545, R58-F1545, S59-F1545, S60-F1545, T61-F1545, T62-F1545, K63-F1545, L64-F1545, Y65-F1545, L66-F1545, A67-F1545, K68-F1545, Q69-F1545, V70-F1545, F71-F1545, V72-F1545, G73-F1545, F74-F1545, L75-F1545, L76-F1545, I77-F1545, L78-F1545, A79-F1545, A80-F1545, I81-F1545, E82-F1545, L83-F1545, A84-F1545, L85-F1545, V86-F1545, L87-F1545, T88-F1545, E89-F1545, D90-F1545, S91-F1545, G92-F1545, Q93-F1545, A94-F1545, T95-F1545, V96-F1545, P97-F1545, A98-F1545, V99-F1545, R100-F1545, Y101-F1545, T102-F1545, N103-F1545, P104-F1545, S105-F1545, L106-F1545, Y107-F1545, L108-F1545, G109-F1545, T110-F1545, W111-F1545, L112-F1545, L113-F1545, V114-F1545, L115-F1545, L116-F1545, I117-F1545, Q118-F1545, Y119-F1545, S120-F1545, R121-F1545, Q122-F1545, W123-F1545, C124-F1545, V125-F1545, Q126-F1545, K127-F1545, N128-F1545, S129-F1545, W130-F1545, F131-F1545, L132-F1545, S133-F1545, L134-F1545, F135-F1545, W136-F1545, I137-F1545, L138-F1545, S139-F1545, I140-F1545, L141-F1545, C142-F1545, G143-F1545, T144-F1545, F145-F1545, Q146-F1545, F147-F1545, Q148-F1545, T149-F1545, L150-F1545, I151-F1545, R152-F1545, T153-F1545, L154-F1545, L155-F1545, Q156-F1545, G157-F1545, D158-F1545, N159-F1545, S160-F1545, N161-F1545, L162-F1545, A163-F1545, Y164-F1545, S165-F1545, C166-F1545, L167-F1545, F168-F1545, F169-F1545, I170-F1545, S171-F1545, Y172-F1545, G173-F1545, F174-F1545, Q175-F1545, I176-F1545, L177-F1545, I178-F1545, L179-F1545, I180-F1545, F181-F1545, S182-F1545, A183-F1545, F184-F1545, S185-F1545, E186-F1545, N187-F1545, N188-F1545, E189-F1545, S190-F1545, S191-F1545, N192-F1545, N193-F1545, P194-F1545, S195-F1545, S196-F1545, I197-F1545, A198-F1545, S199-F1545, F200-F1545, L201-F1545, S202-F1545, S203-F1545, I204-F1545, T205-F1545, Y206-F1545, S207-F1545, W208-F1545, Y209-F1545, D210-F1545, S211-F1545, I212-F1545, I213-F1545, L214-F1545, K215-F1545, G216-F1545, Y217-F1545, K218-F1545, R219-F1545, P220-F1545, L221-F1545, T222-F1545, L223-F1545, E224-F1545, D225-F1545, V226-F1545, W227-F1545, E228-F1545, V229-F1545, D230-F1545, E231-F1545, E232-F1545, M233-F1545, K234-F1545, T235-F1545, K236-F1545, T237-F1545, L238-F1545, V239-F1545, S240-F1545, K241-F1545, F242-F1545, E243-F1545, T244-F1545, H245-F1545, M246-F1545, K247-F1545, R248-F1545, E249-F1545, L250-F1545, Q251-F1545, K252-F1545, A253-F1545, R254-F1545, R255-F1545, A256-F1545, L257-F1545, Q258-F1545, R259-F1545, R260-F1545, Q261-F1545, E262-F1545, K263-F1545, S264-F1545, S265-F1545, Q266-F1545, Q267-F1545, N268-F1545, S269-F1545, G270-F1545, A271-F1545, R272-F1545, L273-F1545, P274-F1545, G275-F1545, L276-F1545, N277-F1545, K278-F1545, N279-F1545, Q280-F1545, S281-F1545, Q282-F1545, S283-F1545, Q284-F1545, D285-F1545, A286-F1545, L287-F1545, V288-F1545, L289-F1545, E290-F1545, D291-F1545, V292-F1545, E293-F1545, K294-F1545, K295-F1545, K296-F1545, K297-F1545, K298-F1545, S299-F1545, G300-F1545, T301-F1545, K302-F1545, K303-F1545, D304-F1545, V305-F1545, P306-F1545, K307-F1545, S308-F1545, W309-F1545, L310-F1545, M311-F1545, K312-F1545, A313-F1545, L314-F1545, F315-F1545, K316-F1545, T317-F1545, F318-F1545, Y319-F1545, M320-F1545, V321-F1545, L322-F1545, L323-F1545, K324-F1545, S325-F1545, F326-F1545, L327-F1545, L328-F1545, K329-F1545, L330-F1545, V331-F1545, N332-F1545, D333-F1545, I334-F1545, F335-F1545, T336-F1545, F337-F1545, V338-F1545, S339-F1545, P340-F1545, Q341-F1545, L342-F1545, L343-F1545, K344-F1545, L345-F1545, L346-F1545, I347-F1545, S348-F1545, F349-F1545, A350-F1545, S351-F1545, D352-F1545, R353-F1545, D354-F1545, T355-F1545, Y356-F1545, L357-F1545, W358-F1545, I359-F1545, G360-F1545, Y361-F1545, L362-F1545, C363-F1545, A364-F1545, I365-F1545, L366-F1545, L367-F1545, F368-F1545, T369-F1545, A370-F1545, A371-F1545, L372-F1545, I373-F1545, Q374-F1545, S375-F1545, F376-F1545, C377-F1545, L378-F1545, Q379-F1545, C380-F1545, Y381-F1545, F382-F1545, Q383-F1545, L384-F1545, C385-F1545, F386-F1545, K387-F1545, L388-F1545, G389-F1545, V390-F1545, K391-F1545, V392-F1545, R393-F1545, T394-F1545, A395-F1545, I396-F1545, M397-F1545, A398-F1545, S399-F1545, V400-F1545, Y401-F1545, K402-F1545, K403-F1545, A404-F1545, L405-F1545, T406-F1545, I407-F1545, S408-F1545, N409-F1545, L410-F1545, A411-F1545, R412-F1545, K413-F1545, E414-F1545, Y415-F1545, T416-F1545, V417-F1545, G418-F1545, E419-F1545, T420-F1545, V421-F1545, N422-F1545, L423-F1545, M424-F1545, S425-F1545, V426-F1545, D427-F1545, A428-F1545, Q429-F1545, K430-F1545, L431-F1545, M432-F1545, D433-F1545, V434-F1545, T435-F1545, N436-F1545, F437-F1545, M438-F1545, H439-F1545, M440-F1545, L441-F1545, W442-F1545, S443-F1545, S444-F1545, V445-F1545, L446-F1545, Q447-F1545, I448-F1545, V449-F1545, L450-F1545, S451-F1545, I452-F1545, F453-F1545, F454-F1545, L455-F1545, W456-F1545, R457-F1545, E458-F1545, L459-F1545, G460-F1545, P461-F1545, S462-F1545, V463-F1545, L464-F1545, A465-F1545, G466-F1545, V467-F1545, G468-F1545, V469-F1545, M470-F1545, V471-F1545, L472-F1545, V473-F1545, I474-F1545, P475-F1545, I476-F1545, N477-F1545, A478-F1545, I479-F1545, L480-F1545, S481-F1545, T482-F1545, K483-F1545, S484-F1545, K485-F1545, T486-F1545, I487-F1545, Q488-F1545, V489-F1545, K490-F1545, N491-F1545, M492-F1545, K493-F1545, N494-F1545, K495-F1545, D496-F1545, K497-F1545, R498-F1545, L499-F1545, K500-F1545, I501-F1545, M502-F1545, N503-F1545, E504-F1545, I505-F1545, L506-F1545, S507-F1545, G508-F1545, I509-F1545, K510-F1545, I511-F1545, L512-F1545, K513-F1545, Y514-F1545, F515-F1545, A516-F1545, W517-F1545, E518-F1545, P519-F1545, S520-F1545, F521-F1545, R522-F1545, D523-F1545, Q524-F1545, V525-F1545, Q526-F1545, N527-F1545, L528-F1545, R529-F1545, K530-F1545, K531-F1545, E532-F1545, L533-F1545, K534-F1545, N535-F1545, L536-F1545, L537-F1545, A538-F1545, F539-F1545, S540-F1545, Q541-F1545, L542-F1545, Q543-F1545, C544-F1545, V545-F1545, V546-F1545, I547-F1545, F548-F1545, V549-F1545, F550-F1545, Q551-F1545, L552-F1545, T553-F1545, P554-F1545, V555-F1545, L556-F1545, V557-F1545, S558-F1545, V559-F1545, V560-F1545, T561-F1545, F562-F1545, S563-F1545, V564-F1545, Y565-F1545, V566-F1545, L567-F1545, V568-F1545, D569-F1545, S570-F1545, N571-F1545, N572-F1545, I573-F1545, L574-F1545, D575-F1545, A576-F1545, Q577-F1545, K578-F1545, A579-F1545, F580-F1545, T581-F1545, S582-F1545, I583-F1545, T584-F1545, L585-F1545, F586-F1545, N587-F1545, I588-F1545, L589-F1545, R590-F1545, F591-F1545, P592-F1545, L593-F1545, S594-F1545, M595-F1545, L596-F1545, P597-F1545, M598-F1545, M599-F1545, I600-F1545, S601-F1545, S602-F1545, M603-F1545, L604-F1545, Q605-F1545, A606-F1545, S607-F1545, V608-F1545, S609-F1545, T610-F1545, E611-F1545, R612-F1545, L613-F1545, E614-F1545, K615-F1545, Y616-F1545, L617-F1545, G618-F1545, G619-F1545, D620-F1545, D621-F1545, L622-F1545, D623-F1545, T624-F1545, S625-F1545, A626-F1545, I627-F1545, R628-F1545, H629-F1545, D630-F1545, C631-F1545, N632-F1545, F633-F1545, D634-F1545, K635-F1545, A636-F1545, M637-F1545, Q638-F1545, F639-F1545, S640-F1545, E641-F1545, A642-F1545, S643-F1545, F644-F1545, T645-F1545, W646-F1545, E647-F1545, H648-F1545, D649-F1545, S650-F1545, E651-F1545, A652-F1545, T653-F1545, V654-F1545, R655-F1545, D656-F1545, V657-F1545, N658-F1545, L659-F1545, D660-F1545, I661-F1545, M662-F1545, A663-F1545, G664-F1545, Q665-F1545, L666-F1545, V667-F1545, A668-F1545, V669-F1545, I670-F1545, G671-F1545, P672-F1545, V673-F1545, G674-F1545, S675-F1545, G676-F1545, K677-F1545, S678-F1545, S679-F1545, L680-F1545, I681-F1545, S682-F1545, A683-F1545, M684-F1545, L685-F1545, G686-F1545, E687-F1545, M688-F1545, E689-F1545, N690-F1545, V691-F1545, H692-F1545, G693-F1545, H694-F1545, I695-F1545, T696-F1545, I697-F1545, K698-F1545, G699-F1545, T700-F1545, T701-F1545, A702-F1545, Y703-F1545, V704-F1545, P705-F1545, Q706-F1545, Q707-F1545, S708-F1545, W709-F1545, I710-F1545, Q711-F1545, N712-F1545, G713-F1545, T714-F1545, I715-F1545, K716-F1545, D717-F1545, N718-F1545, I719-F1545, L720-F1545, F721-F1545, G722-F1545, T723-F1545, E724-F1545, F725-F1545, N726-F1545, E727-F1545, K728-F1545, R729-F1545, Y730-F1545, Q731-F1545, Q732-F1545, V733-F1545, L734-F1545, E735-F1545, A736-F1545, C737-F1545, A738-F1545, L739-F1545, L740-F1545, P741-F1545, D742-F1545, L743-F1545, E744-F1545, M745-F1545, L746-F1545, P747-F1545, G748-F1545, G749-F1545, D750-F1545, L751-F1545, A752-F1545, E753-F1545, I754-F1545, G755-F1545, E756-F1545, K757-F1545, G758-F1545, I759-F1545, N760-F1545, L761-F1545, S762-F1545, G763-F1545, G764-F1545, Q765-F1545, K766-F1545, Q767-F1545, R768-F1545, I769-F1545, S770-F1545, L771-F1545, A772-F1545, R773-F1545, A774-F1545, T775-F1545, Y776-F1545, Q777-F1545, N778-F1545, L779-F1545, D780-F1545, I781-F1545, Y782-F1545, L783-F1545, L784-F1545, D785-F1545, D786-F1545, P787-F1545, L788-F1545, S789-F1545, A790-F1545, V791-F1545, D792-F1545, A793-F1545, H794-F1545, V795-F1545, G796-F1545, K797-F1545, H798-F1545, I799-F1545, F800-F1545, N801-F1545, K802-F1545, V803-F1545, L804-F1545, G805-F1545, P806-F1545, N807-F1545, G808-F1545, L809-F1545, L810-F1545, K811-F1545, G812-F1545, K813-F1545, T814-F1545, R815-F1545, L816-F1545, L817-F1545, V818-F1545, T819-F1545, H820-F1545, S821-F1545, M822-F1545, H823-F1545, F824-F1545, L825-F1545, P826-F1545, Q827-F1545, V828-F1545, D829-F1545, E830-F1545, I831-F1545, V832-F1545, V833-F1545, L834-F1545, G835-F1545, N836-F1545, G837-F1545, T838-F1545, I839-F1545, V840-F1545, E841-F1545, K842-F1545, G843-F1545, S844-F1545, Y845-F1545, S846-F1545, A847-F1545, L848-F1545, L849-F1545, A850-F1545, K851-F1545, K852-F1545, G853-F1545, E854-F1545, F855-F1545, A856-F1545, K857-F1545, N858-F1545, L859-F1545, K860-F1545, T861-F1545, F862-F1545, L863-F1545, R864-F1545, H865-F1545, T866-F1545, G867-F1545, P868-F1545, E869-F1545, E870-F1545, E871-F1545, A872-F1545, T873-F1545, V874-F1545, H875-F1545, D876-F1545, G877-F1545, S878-F1545, E879-F1545, E880-F1545, E881-F1545, D882-F1545, D883-F1545, D884-F1545, Y885-F1545, G886-F1545, L887-F1545, I888-F1545, S889-F1545, S890-F1545, V891-F1545, E892-F1545, E893-F1545, I894-F1545, P895-F1545, E896-F1545, D897-F1545, A898-F1545, A899-F1545, S900-F1545, I901-F1545, T902-F1545, M903-F1545, R904-F1545, R905-F1545, E906-F1545, N907-F1545, S908-F1545, F909-F1545, R910-F1545, R911-F1545, T912-F1545, L913-F1545, S914-F1545, R915-F1545, S916-F1545, S917-F1545, R918-F1545, S919-F1545, N920-F1545, G921-F1545, R922-F1545, H923-F1545, L924-F1545, K925-F1545, S926-F1545, L927-F1545, R928-F1545, N929-F1545, S930-F1545, L931-F1545, K932-F1545, T933-F1545, R934-F1545, N935-F1545, V936-F1545, N937-F1545, S938-F1545, L939-F1545, K940-F1545, E941-F1545, D942-F1545, E943-F1545, E944-F1545, L945-F1545, V946-F1545, K947-F1545, G948-F1545, Q949-F1545, K950-F1545, L951-F1545, I952-F1545, K953-F1545, K954-F1545, E955-F1545, F956-F1545, I957-F1545, E958-F1545, T959-F1545, G960-F1545, K961-F1545, V962-F1545, K963-F1545, F964-F1545, S965-F1545, I966-F1545, Y967-F1545, L968-F1545, E969-F1545, Y970-F1545, L971-F1545, Q972-F1545, A973-F1545, I974-F1545, G975-F1545, L976-F1545, F977-F1545, S978-F1545, I979-F1545, F980-F1545, F981-F1545, I982-F1545, I983-F1545, L984-F1545, A985-F1545, F986-F1545, V987-F1545, M988-F1545, N989-F1545, S990-F1545, V991-F1545, A992-F1545, F993-F1545, I994-F1545, G995-F1545, S996-F1545, N997-F1545, L998-F1545, W999-F1545, L1000-F1545, S1001-F1545, A1002-F1545, W1003-F1545, T1004-F1545, S1005-F1545, D1006-F1545, S1007-F1545, K1008-F1545, I1009-F1545, F1010-F1545, N1011-F1545, S1012-F1545, T1013-F1545, D1014-F1545, Y1015-F1545, P1016-F1545, A1017-F1545, S1018-F1545, Q1019-F1545, R1020-F1545, D1021-F1545, M1022-F1545, R1023-F1545, V1024-F1545, G1025-F1545, V1026-F1545, Y1027-F1545, G1028-F1545, A1029-F1545, L1030-F1545, G1031-F1545, L1032-F1545, A1033-F1545, Q1034-F1545, G1035-F1545, I1036-F1545, F1037-F1545, V1038-F1545, F1039-F1545, I1040-F1545, A1041-F1545, H1042-F1545, F1043-F1545, W1044-F1545, S1045-F1545, A1046-F1545, F1047-F1545, G1048-F1545, F1049-F1545, V1050-F1545, H1051-F1545, A1052-F1545, S1053-F1545, N1054-F1545, I1055-F1545, L1056-F1545, H1057-F1545, K1058-F1545, Q1059-F1545, L1060-F1545, L1061-F1545, N1062-F1545, N1063-F1545, I1064-F1545, L1065-F1545, R1066-F1545, A1067-F1545, P1068-F1545, M1069-F1545, R1070-F1545, F1071-F1545, F1072-F1545, D1073-F1545, T1074-F1545, T1075-F1545, P1076-F1545, T1077-F1545, G1078-F1545, R1079-F1545, I1080-F1545, V1081-F1545, N1082-F1545, R1083-F1545, F1084-F1545, A1085-F1545, G1086-F1545, D1087-F1545, I1088-F1545, S1089-F1545, T1090-F1545, V1091-F1545, D1092-F1545, D1093-F1545, T1094-F1545, L1095-F1545, P1096-F1545, Q1097-F1545, S1098-F1545, L1099-F1545, R1100-F1545, S1101-F1545, W1102-F1545, I1103-F1545, T1104-F1545, C1105-F1545, F1106-F1545, L1107-F1545, G1108-F1545, I1109-F1545, I1110-F1545, S1111-F1545, T1112-F1545, L1113-F1545, V1114-F1545, M1115-F1545, I1116-F1545, C1117-F1545, M1118-F1545, A1119-F1545, T1120-F1545, P1121-F1545, V1122-F1545, F1123-F1545, T1124-F1545, I1125-F1545, I1126-F1545, V1127-F1545, I1128-F1545, P1129-F1545, L1130-F1545, G1131-F1545, I1132-F1545, I1133-F1545, Y1134-F1545, V1135-F1545, S1136-F1545, V1137-F1545, Q1138-F1545, M1139-F1545, F1140-F1545, Y1141-F1545, V1142-F1545, S1143-F1545, T1144-F1545, S1145-F1545, R1146-F1545, Q1147-F1545, L1148-F1545, R1149-F1545, R1150-F1545, L1151-F1545, D1152-F1545, S1153-F1545, V1154-F1545, T1155-F1545, R1156-F1545, S1157-F1545, P1158-F1545, I1159-F1545, Y1160-F1545, S1161-F1545, H1162-F1545, F1163-F1545, S1164-F1545, E1165-F1545, T1166-F1545, V1167-F1545, S1168-F1545, G1169-F1545, L1170-F1545, P1171-F1545, V1172-F1545, I1173-F1545, R1174-F1545, A1175-F1545, F1176-F1545, E1177-F1545, H1178-F1545, Q1179-F1545, Q1180-F1545, R1181-F1545, F1182-F1545, L1183-F1545, K1184-F1545, H1185-F1545, N1186-F1545, E1187-F1545, and/or V2188-F1545 of SEQ ID NO:24. Polynucleotide sequences encoding these polypeptides are also provided. The present invention also encompasses the use of these N-terminal cMOAT (SNP_ID: PS101s1) deletion polypeptides as immunogenic and/or antigenic epitopes as described elsewhere herein.

In preferred embodiments, the following C-terminal cMOAT (SNP_ID: PS101s1) deletion polypeptides are encompassed by the present invention: M1-F1545, M1-K1544, M1-T1543, M1-S1542, M1-N1541, M1-V1540, M1-N1539, M1-E1538, M1-I1537, M1-G1536, M1-A1535, M1-E1534, M1-K1533, M1-A1532, M1-M1531, M1-F1530, M1-Y1529, M1-F1528, M1-P1527, M1-G1526, M1-P1525, M1-I1524, M1-Q1523, M1-L1522, M1-L1521, M1-E1520, M1-E1519, M1-P1518, M1-S1517, M1-G1516, M1-Y1515, M1-E1514, M1-I1513, M1-I1512, M1-K1511, M1-G1510, M1-N1509, M1-D1508, M1-L1507, M1-V1506, M1-M1505, M1-V1504, M1-K1503, M1-D1502, M1-S1501, M1-D1500, M1-M1499, M1-I1498, M1-T1497, M1-H1496, M1-L1495, M1-R1494, M1-H1493, M1-A1492, M1-I1491, M1-T1490, M1-I1489, M1-V1488, M1-T1487, M1-C1486, M1-H1485, M1-A1484, M1-F1483, M1-E1482, M1-N1481, M1-Q1480, M1-I1479, M1-T1478, M1-T1477, M1-Q1476, M1-I1475, M1-L1474, M1-N1473, M1-D1472, M1-T1471, M1-E1470, M1-L1469, M1-D1468, M1-V1467, M1-A1466, M1-A1465, M1-T1464, M1-A1463, M1-E1462, M1-D1461, M1-L1460, M1-V1459, M1-L1458, M1-I1457, M1-K1456, M1-S1455, M1-K1454, M1-R1453, M1-L1452, M1-L1451, M1-A1450, M1-R1449, M1-G1448, M1-L1447, M1-C1446, M1-L1445, M1-L1444, M1-Q1443, M1-R1442, M1-Q1441, M1-G1440, M1-I1439, M1-S1438, M1-L1437, M1-N1436, M1-G1435, M1-G1434, M1-A1433, M1-E1432, M1-T1431, M1-V1430, M1-E1429, M1-H1428, M1-S1427, M1-L1426, M1-G1425, M1-L1424, M1-Q1423, M1-L1422, M1-S1421, M1-A1420, M1-V1419, M1-F1418, M1-S1417, M1-K1416, M1-L1415, M1-H1414, M1-A1413, M1-L1412, M1-E1411, M1-L1410, M1-A1409, M1-K1408, M1-W1407, M1-I1406, M1-E1405, M1-E1404, M1-D1403, M1-S1402, M1-Y1401, M1-N1400, M1-N1399, M1-F1398, M1-P1397, M1-D1396, M1-L1395, M1-N1394, M1-M1393, M1-R1392, M1-L1391, M1-S1390, M1-G1389, M1-S1388, M1-F1387, M1-L1386, M1-I1385, M1-P1384, M1-D1383, M1-Q1382, M1-P1381, M1-I1380, M1-I1379, M1-T1378, M1-L1377, M1-K1376, M1-E1375, M1-R1374, M1-L1373, M1-D1372, M1-H1371, M1-L1370, M1-G1369, M1-I1368, M1-S1367, M1-A1366, M1-I1365, M1-D1364, M1-V1363, M1-G1362, M1-D1361, M1-I1360, M1-I1359, M1-I1358, M1-Q1357, M1-G1356, M1-G1355, M1-A1354, M1-A1353, M1-E1352, M1-L1351, M1-I1350, M1-R1349, M1-F1348, M1-L1347, M1-C1346, M1-N1345, M1-T1344, M1-L1343, M1-S1342, M1-S1341, M1-K1340, M1-G1339, M1-A1338, M1-G1337, M1-T1336, M1-R1335, M1-G1334, M1-V1333, M1-V1332, M1-G1331, M1-I1330, M1-K1329, M1-E1328, M1-M1327, M1-S1326, M1-G1325, M1-I1324, M1-D1323, M1-C1322, M1-T1321, M1-I1320, M1-G1319, M1-R1318, M1-L1317, M1-V1316, M1-L1315, M1-D1314, M1-L1313, M1-E1312, M1-P1311, M1-R1310, M1-Y1309, M1-R1308, M1-V1307, M1-Q1306, M1-Y1305, M1-N1304, M1-N1303, M1-F1302, M1-Q1301, M1-I1300, M1-K1299, M1-G1298, M1-K1297, M1-S1296, M1-P1295, M1-W1294, M1-D1293, M1-P1292, M1-P1291, M1-P1290, M1-R1289, M1-K1288, M1-D1287, M1-T1286, M1-V1285, M1-W1284, M1-P1283, M1-A1282, M1-E1281, M1-N1280, M1-E1279, M1-V1278, M1-K1277, M1-T1276, M1-Y1275, M1-E1274, M1-T1273, M1-I1272, M1-R1271, M1-E1270, M1-V1269, M1-A1268, M1-V1267, M1-I1266, M1-N1265, M1-T1264, M1-E1263, M1-I1262, M1-E1261, M1-S1260, M1-T1259, M1-M1258, M1-R1257, M1-V1256, M1-L1255, M1-W1254, M1-N1253, M1-L1252, M1-T1251, M1-Q1250, M1-T1249, M1-I1248, M1-N1247, M1-L1246, M1-A1245, M1-N1244, M1-S1243, M1-L1242, M1-V1241, M1-F1240, M1-G1239, M1-V1238, M1-T1237, M1-D1236, M1-G1235, M1-S1234, M1-L1233, M1-T1232, M1-D1231, M1-R1230, M1-Y1229, M1-I1228, M1-V1227, M1-M1226, M1-M1225, M1-L1224, M1-A1223, M1-S1222, M1-F1221, M1-F1220, M1-V1219, M1-T1218, M1-L1217, M1-N1216, M1-G1215, M1-V1214, M1-L1213, M1-E1212, M1-L1211, M1-R1210, M1-I1209, M1-A1208, M1-L1207, M1-W1206, M1-R1205, M1-N1204, M1-S1203, M1-T1202, M1-I1201, M1-W1200, M1-S1199, M1-F1198, M1-V1197, M1-C1196, M1-K1195, M1-Q1194, M1-N1193, M1-T1192, M1-D1191, M1-I1190, M1-R1189, and/or M1-V1188 of SEQ ID NO:24. Polynucleotide sequences encoding these polypeptides are also provided. The present invention also encompasses the use of these C-terminal cMOAT (SNP_ID: PS101s1) deletion polypeptides as immunogenic and/or antigenic epitopes as described elsewhere herein.

Alternatively, preferred polypeptides of the present invention may comprise polypeptide sequences corresponding to, for example, internal regions of the cMOAT (SNP_ID: PS101s1) polypeptide (e.g., any combination of both N- and C-terminal cMOAT (SNP_ID: PS101s1) polypeptide deletions) of SEQ ID NO:24. For example, internal regions could be defined by the equation: amino acid NX to amino acid CX, wherein NX refers to any N-terminal deletion polypeptide amino acid of cMOAT (SNP_ID: PS101s1) (SEQ ID NO:24), and where CX refers to any C-terminal deletion polypeptide amino acid of cMOAT (SNP_ID: PS101s1) (SEQ ID NO:24). Polynucleotides encoding these polypeptides are also provided. The present invention also encompasses the use of these polypeptides as an immunogenic and/or antigenic epitope as described elsewhere herein. Preferably, the resulting deletion polypeptide comprises the polypeptide polymorphic loci identified elsewhere herein for cMOAT (SNP_ID: PS101s1), and more preferably comprises the polypeptide polymorphic allele identified elsewhere herein for cMOAT (SNP_ID: PS101s1).

### Features of the Polypeptide Encoded by Gene No: 11

The present invention relates to isolated nucleic acid molecules comprising, or alternatively, consisting of all or a portion of the variant allele of the human cMOAT, ATP-binding cassette sub-family C member 2 gene (SNP_ID: PS101s2) (e.g., wherein reference or wildtype cMOAT gene is exemplified by SEQ ID NO:1). Preferred portions are at least 10, preferably at least 20, preferably at least 40, preferably at least 100, contiguous polynucleotides and comprise a “T at the nucleotide position corresponding to nucleotide 4073 of the cMOAT gene, or a portion of SEQ ID NO:25. Alternatively, preferred portions are at least 10, preferably at least 20, preferably at least 40, preferably at least 100, contiguous polynucleotides and comprise a “C” at the nucleotide position corresponding to nucleotide 4073 of the cMOAT gene, or a portion of SEQ ID NO:25. The invention further relates to isolated gene products, e.g., polypeptides and/or proteins, which are encoded by a nucleic acid molecule comprising all or a portion of the variant allele of the cMOAT gene.

In one embodiment, the invention relates to a method for predicting the likelihood that an individual will have a disorder associated with a “T” at the nucleotide position corresponding to nucleotide position 4073 of SEQ ID NO:25 (or diagnosing or aiding in the diagnosis of such a disorder) comprising the steps of obtaining a DNA sample from an individual to be assessed and determining the nucleotide present at position 4073 of SEQ ID NO:25. The presence of a “T” at this position indicates that the individual has a greater likelihood of having a disorder associated therewith than an individual having a “C” at that position, or a greater likelihood of having more severe symptoms.

Conversely, the invention relates to a method for predicting the likelihood that an individual will have a disorder associated with a “C” at the nucleotide position corresponding to nucleotide position 4073 of SEQ ID NO:25 (or diagnosing or aiding in the diagnosis of such a disorder) comprising the steps of obtaining a DNA sample from an individual to be assessed and determining the nucleotide present at position 4073 of SEQ ID NO:25. The presence of a “C” at this position indicates that the individual has a greater likelihood of having a disorder associated therewith than an individual having a “T” at that position, or a greater likelihood of having more severe symptoms.

Representative disorders which may be detected, diagnosed, identified, treated, prevented, and/or ameliorated by the present invention include, the following, non-limiting diseases and disorders: decreased heptic uptake of administered statins, resistance to beneficial responses of administered statins; resistance to LDL lowering responses to administered statins; resistance to TG lowering responses to administered statins; resistance to HDL elevating responses to administered statins; increased incidence of drug interactions between one or more administered statins; increased incidence of drug and endogenous substance interactions between a statin drug and endogenous substrates of cMOAT including cholate, taurocholate, thyroid hormones T3 and T4, DHEAS, estradiol-17beta-glucuronide, estrone-3-sulfate, prostaglandin E2, thromboxane B2, leukotriene C4, leukotriene E4, and bilirubin and its conjugates; decreased response to HMG-CoA reductase inhibitors; in addition to other disorders referenced herein, which include, for example, any disorder related to high levels of circulating LDL, high levels of TG, and/or low levels of circularing HDL, which include, but are not limited to, cardiovascular diseases, hepatic diseases, angina pectoris, hypertension, heart failure, myocardial infarction, ventricular hypertrophy, vascular diseases, miscrovascular disease, vascular leak syndrome, aneurysm, stroke, embolism, thrombosis, endothelial dysfunction, coronary artery disease, arteriosclerosis, and/or atherosclerosis.

### Features of the Polypeptide Encoded by Gene No: 12

The present invention relates to isolated nucleic acid molecules comprising, or alternatively, consisting of all or a portion of the variant allele of the human cMOAT, ATP-binding cassette sub-family C member 2 -gene (SNP_ID: PS101s4) (e.g., wherein reference or wildtype cMOAT gene is exemplified by SEQ ID NO:1). Preferred portions are at least 10, preferably at least 20, preferably at least 40, preferably at least 100, contiguous polynucleotides and comprise a “T at the nucleotide position corresponding to nucleotide 4211 of the cMOAT gene, or a portion of SEQ ID NO:27. Alternatively, preferred portions are at least 10, preferably at least 20, preferably at least 40, preferably at least 100, contiguous polynucleotides and comprise a “C” at the nucleotide position corresponding to nucleotide 4211 of the cMOAT gene, or a portion of SEQ ID NO:27. The invention further relates to isolated gene products, e.g., polypeptides and/or proteins, which are encoded by a nucleic acid molecule comprising all or a portion of the variant allele of the cMOAT gene.

In one embodiment, the invention relates to a method for predicting the likelihood that an individual will have a disorder associated with a “T” at the nucleotide position corresponding to nucleotide position 4211 of SEQ ID NO:27 (or diagnosing or aiding in the diagnosis of such a disorder) comprising the steps of obtaining a DNA sample from an individual to be assessed and determining the nucleotide present at position 4211 of SEQ ID NO:27. The presence of a “T” at this position indicates that the individual has a greater likelihood of having a disorder associated therewith than an individual having a “C” at that position, or a greater likelihood of having more severe symptoms.

Conversely, the invention relates to a method for predicting the likelihood that an individual will have a disorder associated with a “C” at the nucleotide position corresponding to nucleotide position 4211 of SEQ ID NO:27 (or diagnosing or aiding in the diagnosis of such a disorder) comprising the steps of obtaining a DNA sample from an individual to be assessed and determining the nucleotide present at position 4211 of SEQ ID NO:27. The presence of a “C” at this position indicates that the individual has a greater likelihood of having a disorder associated therewith than an individual having a “T” at that position, or a greater likelihood of having more severe symptoms.

Representative disorders which may be detected, diagnosed, identified, treated, prevented, and/or ameliorated by the present invention include, the following, non-limiting diseases and disorders: decreased heptic uptake of administered statins, resistance to beneficial responses of administered statins; resistance to LDL lowering responses to administered statins; resistance to TG lowering responses to administered statins; resistance to HDL elevating responses to administered statins; increased incidence of drug interactions between one or more administered statins; increased incidence of drug and endogenous substance interactions between a statin drug and endogenous substrates of cMOAT including cholate, taurocholate, thyroid hormones T3 and T4, DHEAS, estradiol-17beta-glucuronide, estrone-3-sulfate, prostaglandin E2, thromboxane B2, leukotriene C4, leukotriene E4, and bilirubin and its conjugates; decreased response to HMG-CoA reductase inhibitors; in addition to other disorders referenced herein, which include, for example, any disorder related to high levels of circulating LDL, high levels of TG, and/or low levels of circularing HDL, which include, but are not limited to, cardiovascular diseases, hepatic diseases, angina pectoris, hypertension, heart failure, myocardial infarction, ventricular hypertrophy, vascular diseases, miscrovascular disease, vascular leak syndrome, aneurysm, stroke, embolism, thrombosis, endothelial dysfunction, coronary artery disease, arteriosclerosis, and/or atherosclerosis.

### Features of the Polypeptide Encoded by Gene No: 13

The present invention relates to isolated nucleic acid molecules comprising, or alternatively, consisting of all or a portion of the variant allele of the human cMOAT, ATP-binding cassette sub-family C member 2 gene (SNP_ID: PS101s5) (e.g., wherein reference or wildtype cMOAT gene is exemplified by SEQ ID NO:1). Preferred portions are at least 10, preferably at least 20, preferably at least 40, preferably at least 100, contiguous polynucleotides and comprise a “T at the nucleotide position corresponding to nucleotide 4163 of the cMOAT gene, or a portion of SEQ ID NO:29. Alternatively, preferred portions are at least 10, preferably at least 20, preferably at least 40, preferably at least 100, contiguous polynucleotides and comprise a “C” at the nucleotide position corresponding to nucleotide 4163 of the cMOAT gene, or a portion of SEQ ID NO:29. The invention further relates to isolated gene products, e.g., polypeptides and/or proteins, which are encoded by a nucleic acid molecule comprising all or a portion of the variant allele of the cMOAT gene.

In one embodiment, the invention relates to a method for predicting the likelihood that an individual will have a disorder associated with a “T” at the nucleotide position corresponding to nucleotide position 4163 of SEQ ID NO:29 (or diagnosing or aiding in the diagnosis of such a disorder) comprising the steps of obtaining a DNA sample from an individual to be assessed and determining the nucleotide present at position 4163 of SEQ ID NO:29. The presence of a “T” at this position indicates that the individual has a greater likelihood of having a disorder associated therewith than an individual having a “C” at that position, or a greater likelihood of having more severe symptoms.

Conversely, the invention relates to a method for predicting the likelihood that an individual will have a disorder associated with a “C” at the nucleotide position corresponding to nucleotide position 4163 of SEQ ID NO:29 (or diagnosing or aiding in the diagnosis of such a disorder) comprising the steps of obtaining a DNA sample from an individual to be assessed and determining the nucleotide present at position 4163 of SEQ ID NO:29. The presence of a “C” at this position indicates that the individual has a greater likelihood of having a disorder associated therewith than an individual having a “T” at that position, or a greater likelihood of having more severe symptoms.

Representative disorders which may be detected, diagnosed, identified, treated, prevented, and/or ameliorated by the present invention include, the following, non-limiting diseases and disorders: decreased heptic uptake of administered statins, resistance to beneficial responses of administered statins; resistance to LDL lowering responses to administered statins; resistance to TG lowering responses to administered statins; resistance to HDL elevating responses to administered statins; increased incidence of drug interactions between one or more administered statins; increased incidence of drug and endogenous substance interactions between a statin drug and endogenous substrates of cMOAT including cholate, taurocholate, thyroid hormones T3 and T4, DHEAS, estradiol-17beta-glucuronide, estrone-3-sulfate, prostaglandin E2, thromboxane B2, leukotriene C4, leukotriene E4, and bilirubin and its conjugates; decreased response to HMG-CoA reductase inhibitors; in addition to other disorders referenced herein, which include, for example, any disorder related to high levels of circulating LDL, high levels of TG, and/or low levels of circulating HDL, which include, but are not limited to, cardiovascular diseases, hepatic diseases, angina pectoris, hypertension, heart failure, myocardial infarction, ventricular hypertrophy, vascular diseases, miscrovascular disease, vascular leak syndrome, aneurysm, stroke, embolism, thrombosis, endothelial dysfunction, coronary artery disease, arteriosclerosis, and/or atherosclerosis.

### Features of the Polypeptide Encoded by Gene No: 14

The present invention relates to isolated nucleic acid molecules comprising, or alternatively, consisting of all or a portion of the variant allele of the human cMOAT, ATP-binding cassette sub-family C member 2 gene (SNP_ID: PS101s6) (e.g., wherein reference or wildtype cMOAT gene is exemplified by SEQ ID NO:1). Preferred portions are at least 10, preferably at least 20, preferably at least 40, preferably at least 100, contiguous polynucleotides and comprise an “A” at the nucleotide position corresponding to nucleotide 4511 of the cMOAT gene, or a portion of SEQ ID NO:31. Alternatively, preferred portions are at least 10, preferably at least 20, preferably at least 40, preferably at least 100, contiguous polynucleotides and comprise a “G” at the nucleotide position corresponding to nucleotide 4511 of the cMOAT gene, or a portion of SEQ ID NO:31. The invention further relates to isolated gene products, e.g., polypeptides and/or proteins, which are encoded by a nucleic acid molecule comprising all or a portion of the variant allele of the cMOAT gene.

In one embodiment, the invention relates to a method for predicting the likelihood that an individual will have a disorder associated with an “A” at the nucleotide position corresponding to nucleotide position 4511 of SEQ ID NO:31 (or diagnosing or aiding in the diagnosis of such a disorder) comprising the steps of obtaining a DNA sample from an individual to be assessed and determining the nucleotide present at position 4511 of SEQ ID NO:31. The presence of an “A” at this position indicates that the individual has a greater likelihood of having a disorder associated therewith than an individual having a “G” at that position, or a greater likelihood of having more severe symptoms.

Conversely, the invention relates to a method for predicting the likelihood that an individual will have a disorder associated with a “G” at the nucleotide position corresponding to nucleotide position 4511 of SEQ ID NO:31 (or diagnosing or aiding in the diagnosis of such a disorder) comprising the steps of obtaining a DNA sample from an individual to be assessed and determining the nucleotide present at position 4511 of SEQ ID NO:31. The presence of a “G” at this position indicates that the individual has a greater likelihood of having a disorder associated therewith than an individual having an “A” at that position, or a greater likelihood of having more severe symptoms.

Representative disorders which may be detected, diagnosed, identified, treated, prevented, and/or ameliorated by the present invention include, the following, non-limiting diseases and disorders: decreased heptic uptake of administered statins, resistance to beneficial responses of administered statins; resistance to LDL lowering responses to administered statins; resistance to TG lowering responses to administered statins; resistance to HDL elevating responses to administered statins; increased incidence of drug interactions between one or more administered statins; increased incidence of drug and endogenous substance interactions between a statin drug and endogenous substrates of cMOAT including cholate, taurocholate, thyroid hormones T3 and T4, DHEAS, estradiol-17beta-glucuronide, estrone-3-sulfate, prostaglandin E2, thromboxane B2, leukotriene C4, leukotriene E4, and bilirubin and its conjugates; decreased response to HMG-CoA reductase inhibitors; in addition to other disorders referenced herein, which include, for example, any disorder related to high levels of circulating LDL, high levels of TG, and/or low levels of circularing HDL, which include, but are not limited to, cardiovascular diseases, hepatic diseases, angina pectoris, hypertension, heart failure, myocardial infarction, ventricular hypertrophy, vascular diseases, miscrovascular disease, vascular leak syndrome, aneurysm, stroke, embolism, thrombosis, endothelial dysfunction, coronary artery disease, arteriosclerosis, and/or atherosclerosis.

### Features of the Polypeptide Encoded by Gene No: 15

The present invention relates to isolated nucleic acid molecules comprising, or alternatively, consisting of all or a portion of the variant allele of the human cMOAT, ATP-binding cassette sub-family C member 2 gene (SNP_ID: PS101s7) (e.g., wherein reference or wildtype cMOAT gene is exemplified by SEQ ID NO:1). Preferred portions are at least 10, preferably at least 20, preferably at least 40, preferably at least 100, contiguous polynucleotides and comprise a “C” at the nucleotide position corresponding to nucleotide 4589 of the cMOAT gene, or a portion of SEQ ID NO:33. Alternatively, preferred portions are at least 10, preferably at least 20, preferably at least 40, preferably at least 100, contiguous polynucleotides and comprise a “T” at the nucleotide position corresponding to nucleotide 4589 of the cMOAT gene, or a portion of SEQ ID NO:33. The invention further relates to isolated gene products, e.g., polypeptides and/or proteins, which are encoded by a nucleic acid molecule comprising all or a portion of the variant allele of the cMOAT gene.

In one embodiment, the invention relates to a method for predicting the likelihood that an individual will have a disorder associated with a “C” at the nucleotide position corresponding to nucleotide position 4589 of SEQ ID NO:33 (or diagnosing or aiding in the diagnosis of such a disorder) comprising the steps of obtaining a DNA sample from an individual to be assessed and determining the nucleotide present at position 4589 of SEQ ID NO:33. The presence of a “C” at this position indicates that the individual has a greater likelihood of having a disorder associated therewith than an individual having a “T” at that position, or a greater likelihood of having more severe symptoms.

Conversely, the invention relates to a method for predicting the likelihood that an individual will have a disorder associated with a “T” at the nucleotide position corresponding to nucleotide position 4589 of SEQ ID NO:33 (or diagnosing or aiding in the diagnosis of such a disorder) comprising the steps of obtaining a DNA sample from an individual to be assessed and determining the nucleotide present at position 4589 of SEQ ID NO:33. The presence of a “T” at this position indicates that the individual has a greater likelihood of having a disorder associated therewith than an individual having a “C” at that position, or a greater likelihood of having more severe symptoms.

Representative disorders which may be detected, diagnosed, identified, treated, prevented, and/or ameliorated by the present invention include, the following, non-limiting diseases and disorders: decreased heptic uptake of administered statins, resistance to beneficial responses of administered statins; resistance to LDL lowering responses to administered statins; resistance to TG lowering responses to administered statins; resistance to HDL elevating responses to administered statins; increased incidence of drug interactions between one or more administered statins; increased incidence of drug and endogenous substance interactions between a statin drug and endogenous substrates of cMOAT including cholate, taurocholate, thyroid hormones T3 and T4, DHEAS, estradiol-17beta-glucuronide, estrone-3-sulfate, prostaglandin E2, thromboxane B2, leukotriene C4, leukotriene E4, and bilirubin and its conjugates; decreased response to HMG-CoA reductase inhibitors; in addition to other disorders referenced herein, which include, for example, any disorder related to high levels of circulating LDL, high levels of TG, and/or low levels of circularing HDL, which include, but are not limited to, cardiovascular diseases, hepatic diseases, angina pectoris, hypertension, heart failure, myocardial infarction, ventricular hypertrophy, vascular diseases, miscrovascular disease, vascular leak syndrome, aneurysm, stroke, embolism, thrombosis, endothelial dysfunction, coronary artery disease, arteriosclerosis, and/or atherosclerosis.

### Features of the Polypeptide Encoded by Gene No: 16

The present invention relates to isolated nucleic acid molecules comprising, or alternatively, consisting of all or a portion of the variant allele of the human cMOAT, ATP-binding cassette sub-family C member 2 gene (SNP_ID: PS101s100) (e.g., wherein reference or wildtype cMOAT gene is exemplified by SEQ ID NO:1). Preferred portions are at least 10, preferably at least 20, preferably at least 40, preferably at least 100, contiguous polynucleotides and comprise a “T at the nucleotide position corresponding to nucleotide 3643 of the cMOAT gene, or a portion of SEQ ID NO:35. Alternatively, preferred portions are at least 10, preferably at least 20, preferably at least 40, preferably at least 100, contiguous polynucleotides and comprise a “G” at the nucleotide position corresponding to nucleotide 3643 of the cMOAT gene, or a portion of SEQ ID NO:35. The invention further relates to isolated gene products, e.g., polypeptides and/or proteins, which are encoded by a nucleic acid molecule comprising all or a portion of the variant allele of the cMOAT gene.

In one embodiment, the invention relates to a method for predicting the likelihood that an individual will have a disorder associated with a “T” at the nucleotide position corresponding to nucleotide position 3643 of SEQ ID NO:35 (or diagnosing or aiding in the diagnosis of such a disorder) comprising the steps of obtaining a DNA sample from an individual to be assessed and determining the nucleotide present at position 3643 of SEQ ID NO:35. The presence of a “T” at this position indicates that the individual has a greater likelihood of having a disorder associated therewith than an individual having a “G” at that position, or a greater likelihood of having more severe symptoms.

Conversely, the invention relates to a method for predicting the likelihood that an individual will have a disorder associated with a “G” at the nucleotide position corresponding to nucleotide position 3643 of SEQ ID NO:35 (or diagnosing or aiding in the diagnosis of such a disorder) comprising the steps of obtaining a DNA sample from an individual to be assessed and determining the nucleotide present at position 3643 of SEQ ID NO:35. The presence of a “G” at this position indicates that the individual has a greater likelihood of having a disorder associated therewith than an individual having a “T” at that position, or a greater likelihood of having more severe symptoms.

The present invention further relates to isolated proteins or polypeptides comprising, or alternatively, consisting of all or a portion of the encoded variant amino acid sequence of the human human cMOAT, solute carrier family 21 member 6 polypeptide (e.g., wherein reference or wildtype human cMOAT, solute carrier family 21 member 6 polypeptide is exemplified by SEQ ID NO:2). Preferred portions are at least 10, preferably at least 20, preferably at least 40, preferably at least 100, contiguous polypeptides and comprises a “L” at the amino acid position corresponding to amino acid 1181 of the cMOAT polypeptide, or a portion of SEQ ID NO:36. Alternatively, preferred portions are at least 10, preferably at least 20, preferably at least 40, preferably at least 100, contiguous polypeptides and comprises a “R” at the amino acid position corresponding to amino acid 1181 of the cMOAT protein, or a portion of SEQ ID NO:36. The invention further relates to isolated nucleic acid molecules encoding such polypeptides or proteins, as well as to antibodies that bind to such proteins or polypeptides.

Representative disorders which may be detected, diagnosed, identified, treated, prevented, and/or ameliorated by the present invention include, the following, non-limiting diseases and disorders: decreased heptic uptake of administered statins, resistance to beneficial responses of administered statins; resistance to LDL lowering responses to administered statins; resistance to TG lowering responses to administered statins; resistance to HDL elevating responses to administered statins; increased incidence of drug interactions between one or more administered statins; increased incidence of drug and endogenous substance interactions between a statin drug and endogenous substrates of cMOAT including cholate, taurocholate, thyroid hormones T3 and T4, DHEAS, estradiol-17beta-glucuronide, estrone-3-sulfate, prostaglandin E2, thromboxane B2, leukotriene C4, leukotriene E4, and bilirubin and its conjugates; decreased response to HMG-CoA reductase inhibitors; in addition to other disorders referenced herein, which include, for example, any disorder related to high levels of circulating LDL, high levels of TG, and/or low levels of circularing HDL, which include, but are not limited to, cardiovascular diseases, hepatic diseases, angina pectoris, hypertension, heart failure, myocardial infarction, ventricular hypertrophy, vascular diseases, miscrovascular disease, vascular leak syndrome, aneurysm, stroke, embolism, thrombosis, endothelial dysfunction, coronary artery disease, arteriosclerosis, and/or atherosclerosis.

In preferred embodiments, the following N-terminal cMOAT (SNP_ID:PS101s10) deletion polypeptides are encompassed by the present invention: M1-F1545, L2-F1545, E3-F1545, K4-F1545, F5-F1545, C6-F1545, N7-F1545, S8-F1545, T9-F1545, F10-F1545, W11-F1545, N12-F1545, S13-F1545, S14-F1545, F15-F1545, L16-F1545, D17-F1545, S18-F1545, P19-F1545, E20-F1545, A21-F1545, D22-F1545, L23-F1545, P24-F1545, L25-F1545, C26-F1545, F27-F1545, E28-F1545, Q29-F1545, T30-F1545, V31-F1545, L32-F1545, V33-F1545, W34-F1545, I35-F1545, P36-F1545, L37-F1545, G38-F1545, F39-F1545, L40-F1545, W41-F1545, L42-F1545, L43-F1545, A44-F1545, P45-F1545, W46-F1545, Q47-F1545, L48-F1545, L49-F1545, H50-F1545, V51-F1545, Y52-F1545, K53-F1545, S54-F1545, R55-F1545, T56-F1545, K57-F1545, R58-F1545, S59-F1545, S60-F1545, T61-F1545, T62-F1545, K63-F1545, L64-F1545, Y65-F1545, L66-F1545, A67-F1545, K68-F1545, Q69-F1545, V70-F1545, F71-F1545, V72-F1545, G73-F1545, F74-F1545, L75-F1545, L76-F1545, I77-F1545, L78-F1545, A79-F1545, A80-F1545, I81-F1545, E82-F1545, L83-F1545, A84-F1545, L85-F1545, V86-F1545, L87-F1545, T88-F1545, E89-F1545, D90-F1545, S91-F1545, G92-F1545, Q93-F1545, A94-F1545, T95-F1545, V96-F1545, P97-F1545, A98-F1545, V99-F1545, R100-F1545, Y101-F1545, T102-F1545, N103-F1545, P104-F1545, S105-F1545, L106-F1545, Y107-F1545, L108-F1545, G109-F1545, T110-F1545, W111-F1545, L112-F1545, L113-F1545, V114-F1545, L115-F1545, L116-F1545, I117-F1545, Q118-F1545, Y119-F1545, S120-F1545, R121-F1545, Q122-F1545, W123-F1545, C124-F1545, V125-F1545, Q126-F1545, K127-F1545, N128-F1545, S129-F1545, W130-F1545, F131-F1545, L132-F1545, S133-F1545, L134-F1545, F135-F1545, W136-F1545, I137-F1545, L138-F1545, S139-F1545, I140-F1545, L141-F1545, C142-F1545, G143-F1545, T144-F1545, F145-F1545, Q146-F1545, F147-F1545, Q148-F1545, T149-F1545, L150-F1545, I151-F1545, R152-F1545, T153-F1545, L154-F1545, L155-F1545, Q156-F1545, G157-F1545, D158-F1545, N159-F1545, S160-F1545, N161-F1545, L162-F1545, A163-F1545, Y164-F1545, S165-F1545, C166-F1545, L167-F1545, F168-F1545, F169-F1545, I170-F1545, S171-F1545, Y172-F1545, G173-F1545, F174-F1545, Q175-F1545, I176-F1545, L177-F1545, I178-F1545, L179-F1545, I180-F1545, F181-F1545, S182-F1545, A183-F1545, F184-F1545, S185-F1545, E186-F1545, N187-F1545, N188-F1545, E189-F1545, S190-F1545, S191-F1545, N192-F1545, N193-F1545, P194-F1545, S195-F1545, S196-F1545, I197-F1545, A198-F1545, S199-F1545, F200-F1545, L201-F1545, S202-F1545, S203-F1545, I204-F1545, T205-F1545, Y206-F1545, S207-F1545, W208-F1545, Y209-F1545, D210-F1545, S211-F1545, I212-F1545, I213-F1545, L214-F1545, K215-F1545, G216-F1545, Y217-F1545, K218-F1545, R219-F1545, P220-F1545, L221-F1545, T222-F1545, L223-F1545, E224-F1545, D225-F1545, V226-F1545, W227-F1545, E228-F1545, V229-F1545, D230-F1545, E231-F1545, E232-F1545, M233-F1545, K234-F1545, T235-F1545, K236-F1545, T237-F1545, L238-F1545, V239-F1545, S240-F1545, K241-F1545, F242-F1545, E243-F1545, T244-F1545, H245-F1545, M246-F1545, K247-F1545, R248-F1545, E249-F1545, L250-F1545, Q251-F1545, K252-F1545, A253-F1545, R254-F1545, R255-F1545, A256-F1545, L257-F1545, Q258-F1545, R259-F1545, R260-F1545, Q261-F1545, E262-F1545, K263-F1545, S264-F1545, S265-F1545, Q266-F1545, Q267-F1545, N268-F1545, S269-F1545, G270-F1545, A271-F1545, R272-F1545, L273-F1545, P274-F1545, G275-F1545, L276-F1545, N277-F1545, K278-F1545, N279-F1545, Q280-F1545, S281-F1545, Q282-F1545, S283-F1545, Q284-F1545, D285-F1545, A286-F1545, L287-F1545, V288-F1545, L289-F1545, E290-F1545, D291-F1545, V292-F1545, E293-F1545, K294-F1545, K295-F1545, K296-F1545, K297-F1545, K298-F1545, S299-F1545, G300-F1545, T301-F1545, K302-F1545, K303-F1545, D304-F1545, V305-F1545, P306-F1545, K307-F1545, S308-F1545, W309-F1545, L310-F1545, M311-F1545, K312-F1545, A313-F1545, L314-F1545, F315-F1545, K316-F1545, T317-F1545, F318-F1545, Y319-F1545, M320-F1545, V321-F1545, L322-F1545, L323-F1545, K324-F1545, S325-F1545, F326-F1545, L327-F1545, L328-F1545, K329-F1545, L330-F1545, V331-F1545, N332-F1545, D333-F1545, I334-F1545, F335-F1545, T336-F1545, F337-F1545, V338-F1545, S339-F1545, P340-F1545, Q341-F1545, L342-F1545, L343-F1545, K344-F1545, L345-F1545, L346-F1545, I347-F1545, S348-F1545, F349-F1545, A350-F1545, S351-F1545, D352-F1545, R353-F1545, D354-F1545, T355-F1545, Y356-F1545, L357-F1545, W358-F1545, I359-F1545, G360-F1545, Y361-F1545, L362-F1545, C363-F1545, A364-F1545, I365-F1545, L366-F1545, L367-F1545, F368-F1545, T369-F1545, A370-F1545, A371-F1545, L372-F1545, I373-F1545, Q374-F1545, S375-F1545, F376-F1545, C377-F1545, L378-F1545, Q379-F1545, C380-F1545, Y381-F1545, F382-F1545, Q383-F1545, L384-F1545, C385-F1545, F386-F1545, K387-F1545, L388-F1545, G389-F1545, V390-F1545, K391-F1545, V392-F1545, R393-F1545, T394-F1545, A395-F1545, I396-F1545, M397-F1545, A398-F1545, S399-F1545, V400-F1545, Y401-F1545, K402-F1545, K403-F1545, A404-F1545, L405-F1545, T406-F1545, L407-F1545, S408-F1545, N409-F1545, L410-F1545, A411-F1545, R412-F1545, K413-F1545, E414-F1545, Y415-F1545, T416-F1545, V417-F1545, G418-F1545, E419-F1545, T420-F1545, V421-F1545, N422-F1545, L423-F1545, M424-F1545, S425-F1545, V426-F1545, D427-F1545, A428-F1545, Q429-F1545, K430-F1545, L431-F1545, M432-F1545, D433-F1545, V434-F1545, T435-F1545, N436-F1545, F437-F1545, M438-F1545, H439-F1545, M440-F1545, L441-F1545, W442-F1545, S443-F1545, S444-F1545, V445-F1545, L446-F1545, Q447-F1545, I448-F1545, V449-F1545, L450-F1545, S451-F1545, I452-F1545, F453-F1545, F454-F1545, L455-F1545, W456-F1545, R457-F1545, E458-F1545, L459-F1545, G460-F1545, P461-F1545, S462-F1545, V463-F1545, L464-F1545, A465-F1545, G466-F1545, V467-F1545, G468-F1545, V469-F1545, M470-F1545, V471-F1545, I472-F1545, V473-F1545, I474-F1545, P475-F1545, I476-F1545, N477-F1545, A478-F1545, I479-F1545, L480-F1545, S481-F1545, T482-F1545, K483-F1545, S484-F1545, K485-F1545, T486-F1545, I487-F1545, Q488-F1545, V489-F1545, K490-F1545, N491-F1545, M492-F1545, K493-F1545, N494-F1545, K495-F1545, D496-F1545, K497-F1545, R498-F1545, L499-F1545, K500-F1545, I501-F1545, M502-F1545, N503-F1545, E504-F1545, I505-F1545, L506-F1545, S507-F1545, G508-F1545, I509-F1545, K510-F1545, I511-F1545, L512-F1545, K513-F1545, Y514-F1545, F515-F1545, A516-F1545, W517-F1545, E518-F1545, P519-F1545, S520-F1545, F521-F1545, R522-F1545, D523-F1545, Q524-F1545, V525-F1545, Q526-F1545, N527-F1545, L528-F1545, R529-F1545, K530-F1545, K531-F1545, E532-F1545, L533-F1545, K534-F1545, N535-F1545, L536-F1545, L537-F1545, A538-F1545, F539-F1545, S540-F1545, Q541-F1545, L542-F1545, Q543-F1545, C544-F1545, V545-F1545, V546-F1545, I547-F1545, F548-F1545, V549-F1545, F550-F1545, Q551-F1545, L552-F1545, T553-F1545, P554-F1545, V555-F1545, L556-F1545, V557-F1545, S558-F1545, V559-F1545, V560-F1545, T561-F1545, F562-F1545, S563-F1545, V564-F1545, Y565-F1545, V566-F1545, L567-F1545, V568-F1545, D569-F1545, S570-F1545, N571-F1545, N572-F1545, I573-F1545, L574-F1545, D575-F1545, A576-F1545, Q577-F1545, K578-F1545, A579-F1545, F580-F1545, T581-F1545, S582-F1545, I583-F1545, T584-F1545, L585-F1545, F586-F1545, N587-F1545, I588-F1545, L589-F1545, R590-F1545, F591-F1545, P592-F1545, L593-F1545, S594-F1545, M595-F1545, L596-F1545, P597-F1545, M598-F1545, M599-F1545, I600-F1545, S601-F1545, S602-F1545, M603-F1545, L604-F1545, Q605-F1545, A606-F1545, S607-F1545, V608-F1545, S609-F1545, T610-F1545, E611-F1545, R612-F1545, L613-F1545, E614-F1545, K615-F1545, Y616-F1545, L617-F1545, G618-F1545, G619-F1545, D620-F1545, D621-F1545, L622-F1545, D623-F1545, T624-F1545, S625-F1545, A626-F1545, I627-F1545, R628-F1545, H629-F1545, D630-F1545, C631-F1545, N632-F1545, F633-F1545, D634-F1545, K635-F1545, A636-F1545, M637-F1545, Q638-F1545, F639-F1545, S640-F1545, E641-F1545, A642-F1545, S643-F1545, F644-F1545, T645-F1545, W646-F1545, E647-F1545, H648-F1545, D649-F1545, S650-F1545, E651-F1545, A652-F1545, T653-F1545, V654-F1545, R655-F1545, D656-F1545, V657-F1545, N658-F1545, L659-F1545, D660-F1545, I661-F1545, M662-F1545, A663-F1545, G664-F1545, Q665-F1545, L666-F1545, V667-F1545, A668-F1545, V669-F1545, I670-F1545, G671-F1545, P672-F1545, V673-F1545, G674-F1545, S675-F1545, G676-F1545, K677-F1545, S678-F1545, S679-F1545, L680-F1545, I681-F1545, S682-F1545, A683-F1545, M684-F1545, L685-F1545, G686-F1545, E687-F1545, M688-F1545, E689-F1545, N690-F1545, V691-F1545, H692-F1545, G693-F1545, H694-F1545, I695-F1545, T696-F1545, I697-F1545, K698-F1545, G699-F1545, T700-F1545, T701-F1545, A702-F1545, Y703-F1545, V704-F1545, P705-F1545, Q706-F1545, Q707-F1545, S708-F1545, W709-F1545, I710-F1545, Q711-F1545, N712-F1545, G713-F1545, T714-F1545, I715-F1545, K716-F1545, D717-F1545, N718-F1545, I719-F1545, L720-F1545, F721-F1545, G722-F1545, T723-F1545, E724-F1545, F725-F1545, N726-F1545, E727-F1545, K728-F1545, R729-F1545, Y730-F1545, Q731-F1545, Q732-F1545, V733-F1545, L734-F1545, E735-F1545, A736-F1545, C737-F1545, A738-F1545, L739-F1545, L740-F1545, P741-F1545, D742-F1545, L743-F1545, E744-F1545, M745-F1545, L746-F1545, P747-F1545, G748-F1545, G749-F1545, D750-F1545, L751-F1545, A752-F1545, E753-F1545, I754-F1545, G755-F1545, E756-F1545, K757-F1545, G758-F1545, I759-F1545, N760-F1545, L761-F1545, S762-F1545, G763-F1545, G764-F1545, Q765-F1545, K766-F1545, Q767-F1545, R768-F1545, I769-F1545, S770-F1545, L771-F1545, A772-F1545, R773-F1545, A774-F1545, T775-F1545, Y776-F1545, Q777-F1545, N778-F1545, L779-F1545, D780-F1545, I781-F1545, Y782-F1545, L783-F1545, L784-F1545, D785-F1545, D786-F1545, P787-F1545, L788-F1545, S789-F1545, A790-F1545, V791-F1545, D792-F1545, A793-F1545, H794-F1545, V795-F1545, G796-F1545, K797-F1545, H798-F1545, I799-F1545, F800-F1545, N801-F1545, K802-F1545, V803-F1545, L804-F1545, G805-F1545, P806-F1545, N807-F1545, G808-F1545, L809-F1545, L810-F1545, K811-F1545, G812-F1545, K813-F1545, T814-F1545, R815-F1545, L816-F1545, L817-F1545, V818-F1545, T819-F1545, H820-F1545, S821-F1545, M822-F1545, H823-F1545, F824-F1545, L825-F1545, P826-F1545, Q827-F1545, V828-F1545, D829-F1545, E830-F1545, I831-F1545, V832-F1545, V833-F1545, L834-F1545, G835-F1545, N836-F1545, G837-F1545, T838-F1545, I839-F1545, V840-F1545, E841-F1545, K842-F1545, G843-F1545, S844-F1545, Y845-F1545, S846-F1545, A847-F1545, L848-F1545, L849-F1545, A850-F1545, K851-F1545, K852-F1545, G853-F1545, E854-F1545, F855-F1545, A856-F1545, K857-F1545, N858-F1545, L859-F1545, K860-F1545, T861-F1545, F862-F1545, L863-F1545, R864-F1545, H865-F1545, T866-F1545, G867-F1545, P868-F1545, E869-F1545, E870-F1545, E871-F1545, A872-F1545, T873-F1545, V874-F1545, H875-F1545, D876-F1545, G877-F1545, S878-F1545, E879-F1545, E880-F1545, E881-F1545, D882-F1545, D883-F1545, D884-F1545, Y885-F1545, G886-F1545, L887-F1545, I888-F1545, S889-F1545, S890-F1545, V891-F1545, E892-F1545, E893-F1545, I894-F1545, P895-F1545, E896-F1545, D897-F1545, A898-F1545, A899-F1545, S900-F1545, I901-F1545, T902-F1545, M903-F1545, R904-F1545, R905-F1545, E906-F1545, N907-F1545, S908-F1545, F909-F1545, R910-F1545, R911-F1545, T912-F1545, L913-F1545, S914-F1545, R915-F1545, S916-F1545, S917-F1545, R918-F1545, S919-F1545, N920-F1545, G921-F1545, R922-F1545, H923-F1545, L924-F1545, K925-F1545, S926-F1545, L927-F1545, R928-F1545, N929-F1545, S930-F1545, L931-F1545, K932-F1545, T933-F1545, R934-F1545, N935-F1545, V936-F1545, N937-F1545, S938-F1545, L939-F1545, K940-F1545, E941-F1545, D942-F1545, E943-F1545, E944-F1545, L945-F1545, V946-F1545, K947-F1545, G948-F1545, Q949-F1545, K950-F1545, L951-F1545, I952-F1545, K953-F1545, K954-F1545, E955-F1545, F956-F1545, I957-F1545, E958-F1545, T959-F1545, G960-F1545, K961-F1545, V962-F1545, K963-F1545, F964-F1545, S965-F1545, I966-F1545, Y967-F1545, L968-F1545, E969-F1545, Y970-F1545, L971-F1545, Q972-F1545, A973-F1545, I974-F1545, G975-F1545, L976-F1545, F977-F1545, S978-F1545, I979-F1545, F980-F1545, F981-F1545, I982-F1545, I983-F1545, L984-F1545, A985-F1545, F986-F1545, V987-F1545, M988-F1545, N989-F1545, S990-F1545, V991-F1545, A992-F1545, F993-F1545, I994-F1545, G995-F1545, S996-F1545, N997-F1545, L998-F1545, W999-F1545, L1000-F1545, S1001-F1545, A1002-F1545, W1003-F1545, T1004-F1545, S1005-F1545, D1006-F1545, S1007-F1545, K1008-F1545, I1009-F1545, F1010-F1545, N1011-F1545, S1012-F1545, T1013-F1545, D1014-F1545, Y1015-F1545, P1016-F1545, A1017-F1545, S1018-F1545, Q1019-F1545, R1020-F1545, D1021-F1545, M1022-F1545, R1023-F1545, V1024-F1545, G1025-F1545, V1026-F1545, Y1027-F1545, G1028-F1545, A1029-F1545, L1030-F1545, G1031-F1545, L1032-F1545, A1033-F1545, Q1034-F1545, G1035-F1545, I1036-F1545, F1037-F1545, V1038-F1545, F1039-F1545, I1040-F1545, A1041-F1545, H1042-F1545, F1043-F1545, W1044-F1545, S1045-F1545, A1046-F1545, F1047-F1545, G1048-F1545, F1049-F1545, V1050-F1545, H1051-F1545, A1052-F1545, S1053-F1545, N1054-F1545, I1055-F1545, L1056-F1545, H1057-F1545, K1058-F1545, Q1059-F1545, L1060-F1545, L1061-F1545, N1062-F1545, N1063-F1545, I1064-F1545, L1065-F1545, R1066-F1545, A1067-F1545, P1068-F1545, M1069-F1545, R1070-F1545, F1071-F1545, F1072-F1545, D1073-F1545, T1074-F1545, T1075-F1545, P1076-F1545, T1077-F1545, G1078-F1545, R1079-F1545, I1080-F1545, V1081-F1545, N1082-F1545, R1083-F1545, F1084-F1545, A1085-F1545, G1086-F1545, D1087-F1545, I1088-F1545, S1089-F1545, T1090-F1545, V1091-F1545, D1092-F1545, D1093-F1545, T1094-F1545, L1095-F1545, P1096-F1545, Q1097-F1545, S1098-F1545, L1099-F1545, R1100-F1545, S1101-F1545, W1102-F1545, I1103-F1545, T1104-F1545, C1105-F1545, F1106-F1545, L1107-F1545, G1108-F1545, I1109-F1545, I1110-F1545, S111-F1545, T1112-F1545, L1113-F1545, V1114-F1545, M1115-F1545, I1116-F1545, C1117-F1545, M1118-F1545, A1119-F1545, T1120-F1545, P1121-F1545, V1122-F1545, F1123-F1545, T1124-F1545, I1125-F1545, I1126-F1545, V1127-F1545, I1128-F1545, P1129-F1545, L1130-F1545, G1131-F1545, I1132-F1545, I1133-F1545, Y1134-F1545, V1135-F1545, S1136-F1545, V1137-F1545, Q1138-F1545, M1139-F1545, F1140-F1545, Y1141-F1545, V1142-F1545, S1143-F1545, T1144-F1545, S1145-F1545, R1146-F1545, Q1147-F1545, L1148-F1545, R1149-F1545, R1150-F1545, L1151-F1545, D1152-F1545, S1153-F1545, V1154-F1545, T1155-F1545, R1156-F1545, S1157-F1545, P1158-F1545, I1159-F1545, Y1160-F1545, S1161-F1545, H1162-F1545, F1163-F1545, S1164-F1545, E1165-F1545, T1166-F1545, V1167-F1545, S1168-F1545, G1169-F1545, L1170-F1545, P1171-F1545, V1172-F1545, I1173-F1545, R1174-F1545, A1175-F1545, F1176-F1545, E1177-F1545, H1178-F1545, Q1179-F1545, Q1180-F1545, and/or L1181-F1545 of SEQ ID NO:36. Polynucleotide sequences encoding these polypeptides are also provided. The present invention also encompasses the use of these N-terminal cMOAT (SNP_ID:PS101s10) deletion polypeptides as immunogenic and/or antigenic epitopes as described elsewhere herein.

In preferred embodiments, the following C-terminal cMOAT (SNP_ID:PS101s10) deletion polypeptides are encompassed by the present invention: M1-F1545, M1-K1544, M1-T1543, M1-S1542, M1-N1541, M1-V1540, M1-N1539, M1-E1538, M1-I1537, M1-G1536, M1-A1535, M1-E1534, M1-K1533, M1-A1532, M1-M1531, M1-F1530, M1-Y1529, M1-F1528, M1-P1527, M1-G1526, M1-P1525, M1-I1524, M1-Q1523, M1-L1522, M1-L1521, M1-E1520, M1-E1519, M1-P1518, M1-S1517, M1-G1516, M1-Y1515, M1-E1514, M1-I1513, M1-I1512, M1-K1511, M1-G1510, M1-N1509, M1-D1508, M1-L1507, M1-V1506, M1-M1505, M1-V1504, M1-K1503, M1-D1502, M1-S1501, M1-D1500, M1-M1499, M1-I1498, M1-T1497, M1-H1496, M1-L1495, M1-R1494, M1-H1493, M1-A1492, M1-I1491, M1-T1490, M1-I1489, M1-V1488, M1-T1487, M1-C1486, M1-H1485, M1-A1484, M1-F1483, M1-E1482, M1-N1481, M1-Q1480, M1-I1479, M1-T1478, M1-T1477, M1-Q1476, M1-I1475, M1-L1474, M1-N1473, M1-D1472, M1-T1471, M1-E1470, M1-L1469, M1-D1468, M1-V1467, M1-A1466, M1-A1465, M1-T1464, M1-A1463, M1-E1462, M1-D1461, M1-L1460, M1-V1459, M1-L1458, M1-I1457, M1-K1456, M1-S1455, M1-K1454, M1-R1453, M1-L1452, M1-L1451, M1-A1450, M1-R1449, M1-G1448, M1-L1447, M1-C1446, M1-L1445, M1-L1444, M1-Q1443, M1-R1442, M1-Q1441, M1-G1440, M1-I1439, M1-S1438, M1-L1437, M1-N1436, M1-G1435, M1-G1434, M1-A1433, M1-E1432, M1-T1431, M1-V1430, M1-E1429, M1-H1428, M1-S1427, M1-L1426, M1-G1425, M1-L1424, M1-Q1423, M1-L1422, M1-S1421, M1-A1420, M1-V1419, M1-F1418, M1-S1417, M1-K1416, M1-L1415, M1-H1414, M1-A1413, M1-L1412, M1-E1411, M1-L1410, M1-A1409, M1-K1408, M1-W1407, M1-I1406, M1-E1405, M1-E1404, M1-D1403, M1-S1402, M1-Y1401, M1-N1400, M1-N1399, M1-F1398, M1-P1397, M1-D1396, M1-L1395, M1-N1394, M1-M1393, M1-R1392, M1-L1391, M1-S1390, M1-G1389, M1-S1388, M1-F1387, M1-L1386, M1-I1385, M1-P1384, M1-D1383, M1-Q1382, M1-P1381, M1-I1380, M1-I1379, M1-T1378, M1-L1377, M1-K1376, M1-E1375, M1-R1374, M1-L1373, M1-D1372, M1-H1371, M1-L1370, M1-G1369, M1-I1368, M1-S1367, M1-A1366, M1-I1365, M1-D1364, M1-V1363, M1-G1362, M1-D1361, M1-I1360, M1-I1359, M1-I1358, M1-Q1357, M1-G1356, M1-G1355, M1-A1354, M1-A1353, M1-E1352, M1-L1351, M1-I1350, M1-R1349, M1-F1348, M1-L1347, M1-C1346, M1-N1345, M1-T1344, M1-L1343, M1-S1342, M1-S1341, M1-K1340, M1-G1339, M1-A1338, M1-G1337, M1-T1336, M1-R1335, M1-G1334, M1-V1333, M1-V1332, M1-G1331, M1-I1330, M1-K1329, M1-E1328, M1-M1327, M1-S1326, M1-G1325, M1-I1324, M1-D1323, M1-C1322, M1-T1321, M1-I1320, M1-G1319, M1-R1318, M1-L1317, M1-V1316, M1-L1315, M1-D1314, M1-L1313, M1-E1312, M1-P1311, M1-R1310, M1-Y1309, M1-R1308, M1-V1307, M1-Q1306, M1-Y1305, M1-N1304, M1-N1303, M1-F1302, M1-Q1301, M1-I1300, M1-K1299, M1-G1298, M1-K1297, M1-S1296, M1-P1295, M1-W1294, M1-D1293, M1-P1292, M1-P1291, M1-P1290, M1-R1289, M1-K1288, M1-D1287, M1-T1286, M1-V1285, M1-W1284, M1-P1283, M1-A1282, M1-E1281, M1-N1280, M1-E1279, M1-V1278, M1-K1277, M1-T1276, M1-Y1275, M1-E1274, M1-T1273, M1-I1272, M1-R1271, M1-E1270, M1-V1269, M1-A1268, M1-V1267, M1-I1266, M1-N1265, M1-T1264, M1-E1263, M1-I1262, M1-E1261, M1-S1260, M1-T1259, M1-M1258, M1-R1257, M1-V1256, M1-L1255, M1-W1254, M1-N1253, M1-L1252, M1-T1251, M1-Q1250, M1-T1249, M1-I1248, M1-N1247, M1-L1246, M1-A1245, M1-N1244, M1-S1243, M1-L1242, M1-V1241, M1-F1240, M1-G1239, M1-V1238, M1-T1237, M1-D1236, M1-G1235, M1-S1234, M1-L1233, M1-T1232, M1-D1231, M1-R1230, M1-Y1229, M1-I1228, M1-V1227, M1-M1226, M1-M1225, M1-L1224, M1-A1223, M1-S1222, M1-F1221, M1-F1220, M1-V1219, M1-T1218, M1-L1217, M1-N1216, M1-G1215, M1-V1214, M1-L1213, M1-E1212, M1-L1211, M1-R1210, M1-I1209, M1-A1208, M1-L1207, M1-W1206, M1-R1205, M1-N1204, M1-S1203, M1-T1202, M1-I1201, M1-W1200, M1-S1199, M1-F1198, M1-V1197, M1-C1196, M1-K1195, M1-Q1194, M1-N1193, M1-T1192, M1-D1191, M1-I1190, M1-R1189, M1-E1188, M1-E1187, M1-N1186, M1-H1185, M1-K1184, M1-L1183, M1-F1182, and/or M1-L1181 of SEQ ID NO:36. Polynucleotide sequences encoding these polypeptides are also provided. The present invention also encompasses the use of these C-terminal cMOAT (SNP_ID:PS101s10) deletion polypeptides as immunogenic and/or antigenic epitopes as described elsewhere herein.

Alternatively, preferred polypeptides of the present invention may comprise polypeptide sequences corresponding to, for example, internal regions of the cMOAT (SNP_ID: PS101s10) polypeptide (e.g., any combination of both N- and C-terminal cMOAT (SNP_ID: PS101s10) polypeptide deletions) of SEQ ID NO:36. For example, internal regions could be defined by the equation: amino acid NX to amino acid CX, wherein NX refers to any N-terminal deletion polypeptide amino acid of cMOAT (SNP_ID: PS101s10) (SEQ ID NO:36), and where CX refers to any C-terminal deletion polypeptide amino acid of cMOAT (SNP_ID: PS101s10) (SEQ ID NO:36). Polynucleotides encoding these polypeptides are also provided. The present invention also encompasses the use of these polypeptides as an immunogenic and/or antigenic epitope as described elsewhere herein. Preferably, the resulting deletion polypeptide comprises the polypeptide polymorphic loci identified elsewhere herein for cMOAT (SNP_ID: PS101s10), and more preferably comprises the polypeptide polymorphic allele identified elsewhere herein for cMOAT (SNP_ID: PS101s10).

### Features of the Polypeptide Encoded by Gene No: 17

The present invention relates to isolated nucleic acid molecules comprising, or alternatively, consisting of all or a portion of the variant allele of the human cMOAT, ATP-binding cassette sub-family C member 2 gene (SNP_ID: PS101s110) (e.g., wherein reference or wildtype cMOAT gene is exemplified by SEQ ID NO:1). Preferred portions are at least 10, preferably at least 20, preferably at least 40, preferably at least 100, contiguous polynucleotides and comprise a “G” at the nucleotide position corresponding to nucleotide 2983 of the cMOAT gene, or a portion of SEQ ID NO:37. Alternatively, preferred portions are at least 10, preferably at least 20, preferably at least 40, preferably at least 100, contiguous polynucleotides and comprise an “A” at the nucleotide position corresponding to nucleotide 2983 of the cMOAT gene, or a portion of SEQ ID NO:37. The invention further relates to isolated gene products, e.g., polypeptides and/or proteins, which are encoded by a nucleic acid molecule comprising all or a portion of the variant allele of the cMOAT gene.

In one embodiment, the invention relates to a method for predicting the likelihood that an individual will have a disorder associated with a “G” at the nucleotide position corresponding to nucleotide position 2983 of SEQ ID NO:37 (or diagnosing or aiding in the diagnosis of such a disorder) comprising the steps of obtaining a DNA sample from an individual to be assessed and determining the nucleotide present at position 2983 of SEQ ID NO:37. The presence of a “G” at this position indicates that the individual has a greater likelihood of having a disorder associated therewith than an individual having an “A” at that position, or a greater likelihood of having more severe symptoms.

Conversely, the invention relates to a method for predicting the likelihood that an individual will have a disorder associated with an “A” at the nucleotide position corresponding to nucleotide position 2983 of SEQ ID NO:37 (or diagnosing or aiding in the diagnosis of such a disorder) comprising the steps of obtaining a DNA sample from an individual to be assessed and determining the nucleotide present at position 2983 of SEQ ID NO:37. The presence of an “A” at this position indicates that the individual has a greater likelihood of having a disorder associated therewith than an individual having a “G” at that position, or a greater likelihood of having more severe symptoms.

The present invention further relates to isolated proteins or polypeptides comprising, or alternatively, consisting of all or a portion of the encoded variant amino acid sequence of the human human cMOAT, solute carrier family 21 member 6 polypeptide (e.g., wherein reference or wildtype human cMOAT, solute carrier family 21 member 6 polypeptide is exemplified by SEQ ID NO:2). Preferred portions are at least 10, preferably at least 20, preferably at least 40, preferably at least 100, contiguous polypeptides and comprises a “R” at the amino acid position corresponding to amino acid 961 of the cMOAT polypeptide, or a portion of SEQ ID NO:38. Alternatively, preferred portions are at least 10, preferably at least 20, preferably at least 40, preferably at least 100, contiguous polypeptides and comprises a “K” at the amino acid position corresponding to amino acid 961 of the cMOAT protein, or a portion of SEQ ID NO:38. The invention further relates to isolated nucleic acid molecules encoding such polypeptides or proteins, as well as to antibodies that bind to such proteins or polypeptides.

Representative disorders which may be detected, diagnosed, identified, treated, prevented, and/or ameliorated by the present invention include, the following, non-limiting diseases and disorders: decreased heptic uptake of administered statins, resistance to beneficial responses of administered statins; resistance to LDL lowering responses to administered statins; resistance to TG lowering responses to administered statins; resistance to HDL elevating responses to administered statins; increased incidence of drug interactions between one or more administered statins; increased incidence of drug and endogenous substance interactions between a statin drug and endogenous substrates of cMOAT including cholate, taurocholate, thyroid hormones T3 and T4, DHEAS, estradiol-17beta-glucuronide, estrone-3-sulfate, prostaglandin E2, thromboxane B2, leukotriene C4, leukotriene E4, and bilirubin and its conjugates; decreased response to HMG-CoA reductase inhibitors; in addition to other disorders referenced herein, which include, for example, any disorder related to high levels of circulating LDL, high levels of TG, and/or low levels of circularing HDL, which include, but are not limited to, cardiovascular diseases, hepatic diseases, angina pectoris, hypertension, heart failure, myocardial infarction, ventricular hypertrophy, vascular diseases, miscrovascular disease, vascular leak syndrome, aneurysm, stroke, embolism, thrombosis, endothelial dysfunction, coronary artery disease, arteriosclerosis, and/or atherosclerosis.

In preferred embodiments, the following N-terminal cMOAT (SNP_ID:PS101s11) deletion polypeptides are encompassed by the present invention: M1-F1545, L2-F1545, E3-F1545, K4-F1545, F5-F1545, C6-F1545, N7-F1545, S8-F1545, T9-F1545, F10-F1545, W11-F1545, N12-F1545, S13-F1545, S14-F1545, F15-F1545, L16-F1545, D17-F1545, S18-F1545, P19-F1545, E20-F1545, A21-F1545, D22-F1545, L23-F1545, P24-F1545, L25-F1545, C26-F1545, F27-F1545, E28-F1545, Q29-F1545, T30-F1545, V31-F1545, L32-F1545, V33-F1545, W34-F1545, I35-F1545, P36-F1545, L37-F1545, G38-F1545, F39-F1545, L40-F1545, W41-F1545, LA2-F1545, L43-F1545, A44-F1545, P45-F1545, W46-F1545, Q47-F1545, L48-F1545, LA9-F1545, H50-F1545, V51-F1545, Y52-F1545, K53-F1545, S54-F1545, R55-F1545, T56-F1545, K57-F1545, R58-F1545, S59-F1545, S60-F1545, T61-F1545, T62-F1545, K63-F1545, L64-F1545, Y65-F1545, L66-F1545, A67-F1545, K68-F1545, Q69-F1545, V70-F1545, F71-F1545, V72-F1545, G73-F1545, F74-F1545, L75-F1545, L76-F1545, I77-F1545, L78-F1545, A79-F1545, A80-F1545, I81-F1545, E82-F1545, L83-F1545, A84-F1545, L85-F1545, V86-F1545, L87-F1545, T88-F1545, E89-F1545, D90-F1545, S91-F1545, G92-F1545, Q93-F1545, A94-F1545, T95-F1545, V96-F1545, P97-F1545, A98-F1545, V99-F1545, R100-F1545, Y101-F1545, T102-F1545, N103-F1545, P104-F1545, S105-F1545, L106-F1545, Y107-F1545, L108-F1545, G109-F1545, T110-F1545, W111-F1545, L112-F1545, L113-F1545, V114-F1545, L115-F1545, L116-F1545, I117-F1545, Q118-F1545, Y119-F1545, S120-F1545, R121-F1545, Q122-F1545, W123-F1545, C124-F1545, V125-F1545, Q126-F1545, K127-F1545, N128-F1545, S129-F1545, W130-F1545, F131-F1545, L132-F1545, S133-F1545, L134-F1545, F135-F1545, W136-F1545, I137-F1545, L138-F1545, S139-F1545, I140-F1545, L141-F1545, C142-F1545, G143-F1545, T144-F1545, F145-F1545, Q146-F1545, F147-F1545, Q148-F1545, T149-F1545, L150-F1545, I151-F1545, R152-F1545, T153-F1545, L154-F1545, L155-F1545, Q156-F1545, G157-F1545, D158-F1545, N159-F1545, S160-F1545, N161-F1545, L162-F1545, A163-F1545, Y164-F1545, S165-F1545, C166-F1545, L167-F1545, F168-F1545, F169-F1545, I170-F1545, S171-F1545, Y172-F1545, G173-F1545, F174-F1545, Q175-F1545, I176-F1545, L177-F1545, I178-F1545, L179-F1545, I180-F1545, F181-F1545, S182-F1545, A183-F1545, F184-F1545, S185-F1545, E186-F1545, N187-F1545, N188-F1545, E189-F1545, S190-F1545, S191-F1545, N192-F1545, N193-F1545, P194-F1545, S195-F1545, S196-F1545, I197-F1545, A198-F1545, S199-F1545, F200-F1545, L201-F1545, S202-F1545, S203-F1545, I204-F1545, T205-F1545, Y206-F1545, S207-F1545, W208-F1545, Y209-F1545, D210-F1545, S211-F1545, I212-F1545, I213-F1545, L214-F1545, K215-F1545, G216-F1545, Y217-F1545, K218-F1545, R219-F1545, P220-F1545, L221-F1545, T222-F1545, L223-F1545, E224-F1545, D225-F1545, V226-F1545, W227-F1545, E228-F1545, V229-F1545, D230-F1545, E231-F1545, E232-F1545, M233-F1545, K234-F1545, T235-F1545, K236-F1545, T237-F1545, L238-F1545, V239-F1545, S240-F1545, K241-F1545, F242-F1545, E243-F1545, T244-F1545, H245-F1545, M246-F1545, K247-F1545, R248-F1545, E249-F1545, L250-F1545, Q251-F1545, K252-F1545, A253-F1545, R254-F1545, R255-F1545, A256-F1545, L257-F1545, Q258-F1545, R259-F1545, R260-F1545, Q261-F1545, E262-F1545, K263-F1545, S264-F1545, S265-F1545, Q266-F1545, Q267-F1545, N268-F1545, S269-F1545, G270-F1545, A271-F1545, R272-F1545, L273-F1545, P274-F1545, G275-F1545, L276-F1545, N277-F1545, K278-F1545, N279-F1545, Q280-F1545, S281-F1545, Q282-F1545, S283-F1545, Q284-F1545, D285-F1545, A286-F1545, L287-F1545, V288-F1545, L289-F1545, E290-F1545, D291-F1545, V292-F1545, E293-F1545, K294-F1545, K295-F1545, K296-F1545, K297-F1545, K298-F1545, S299-F1545, G300-F1545, T301-F1545, K302-F1545, K303-F1545, D304-F1545, V305-F1545, P306-F1545, K307-F1545, S308-F1545, W309-F1545, L310-F1545, M311-F1545, K312-F1545, A313-F1545, L314-F1545, F315-F1545, K316-F1545, T317-F1545, F318-F1545, Y319-F1545, M320-F1545, V321-F1545, L322-F1545, L323-F1545, K324-F1545, S325-F1545, F326-F1545, L327-F1545, L328-F1545, K329-F1545, L330-F1545, V331-F1545, N332-F1545, D333-F1545, I334-F1545, F335-F1545, T336-F1545, F337-F1545, V338-F1545, S339-F1545, P340-F1545, Q341-F1545, L342-F1545, L343-F1545, K344-F1545, L345-F1545, L346-F1545, I347-F1545, S348-F1545, F349-F1545, A350-F1545, S351-F1545, D352-F1545, R353-F1545, D354-F1545, T355-F1545, Y356-F1545, L357-F1545, W358-F1545, I359-F1545, G360-F1545, Y361-F1545, L362-F1545, C363-F1545, A364-F1545, I365-F1545, L366-F1545, L367-F1545, F368-F1545, T369-F1545, A370-F1545, A371-F1545, L372-F1545, I373-F1545, Q374-F1545, S375-F1545, F376-F1545, C377-F1545, L378-F1545, Q379-F1545, C380-F1545, Y381-F1545, F382-F1545, Q383-F1545, L384-F1545, C385-F1545, F386-F1545, K387-F1545, L388-F1545, G389-F1545, V390-F1545, K391-F1545, V392-F1545, R393-F1545, T394-F1545, A395-F1545, I396-F1545, M397-F1545, A398-F1545, S399-F1545, V400-F1545, Y401-F1545, K402-F1545, K403-F1545, A404-F1545, L405-F1545, T406-F1545, L407-F1545, S408-F1545, N409-F1545, L410-F1545, A411-F1545, R412-F1545, K413-F1545, E414-F1545, Y415-F1545, T416-F1545, V417-F1545, G418-F1545, E419-F1545, T420-F1545, V421-F1545, N422-F1545, L423-F1545, M424-F1545, S425-F1545, V426-F1545, D427-F1545, A428-F1545, Q429-F1545, K430-F1545, L431-F1545, M432-F1545, D433-F1545, V434-F1545, T435-F1545, N436-F1545, F437-F1545, M438-F1545, H439-F1545, M440-F1545, L441-F1545, W442-F1545, S443-F1545, S444-F1545, V445-F1545, L446-F1545, Q447-F1545, I448-F1545, V449-F1545, L450-F1545, S451-F1545, I452-F1545, F453-F1545, F454-F1545, L455-F1545, W456-F1545, R457-F1545, E458-F1545, L459-F1545, G460-F1545, P461-F1545, S462-F1545, V463-F1545, L464-F1545, A465-F1545, G466-F1545, V467-F1545, G468-F1545, V469-F1545, M470-F1545, V471-F1545, L472-F1545, V473-F1545, I474-F1545, P475-F1545, I476-F1545, N477-F1545, A478-F1545, I479-F1545, L480-F1545, S481-F1545, T482-F1545, K483-F1545, S484-F1545, K485-F1545, T486-F1545, I487-F1545, Q488-F1545, V489-F1545, K490-F1545, N491-F1545, M492-F1545, K493-F1545, N494-F1545, K495-F1545, D496-F1545, K497-F1545, R498-F1545, L499-F1545, K500-F1545, I501-F1545, M502-F1545, N503-F1545, E504-F1545, I505-F1545, L506-F1545, S507-F1545, G508-F1545, I509-F1545, K510-F1545, I511-F1545, L512-F1545, K513-F1545, Y514-F1545, F515-F1545, A516-F1545, W517-F1545, E518-F1545, P519-F1545, S520-F1545, F521-F1545, R522-F1545, D523-F1545, Q524-F1545, V525-F1545, Q526-F1545, N527-F1545, L528-F1545, R529-F1545, K530-F1545, K531-F1545, E532-F1545, L533-F1545, K534-F1545, N535-F1545, L536-F1545, L537-F1545, A538-F1545, F539-F1545, S540-F1545, Q541-F1545, L542-F1545, Q543-F1545, C544-F1545, V545-F1545, V546-F1545, I547-F1545, F548-F1545, V549-F1545, F550-F1545, Q551-F1545, L552-F1545, T553-F1545, P554-F1545, V555-F1545, L556-F1545, V557-F1545, S558-F1545, V559-F1545, V560-F1545, T561-F1545, F562-F1545, S563-F1545, V564-F1545, Y565-F1545, V566-F1545, L567-F1545, V568-F1545, D569-F1545, S570-F1545, N571-F1545, N572-F1545, I573-F1545, L574-F1545, D575-F1545, A576-F1545, Q577-F1545, K578-F1545, A579-F1545, F580-F1545, T581-F1545, S582-F1545, I583-F1545, T584-F1545, L585-F1545, F586-F1545, N587-F1545, I588-F1545, L589-F1545, R590-F1545, F591-F1545, P592-F1545, L593-F1545, S594-F1545, M595-F1545, L596-F1545, P597-F1545, M598-F1545, M599-F1545, I600-F1545, S601-F1545, S602-F1545, M603-F1545, L604-F1545, Q605-F1545, A606-F1545, S607-F1545, V608-F1545, S609-F1545, T610-F1545, E611-F1545, R612-F1545, L613-F1545, E614-F1545, K615-F1545, Y616-F1545, L617-F1545, G618-F1545, G619-F1545, D620-F1545, D621-F1545, L622-F1545, D623-F1545, T624-F1545, S625-F1545, A626-F1545, I627-F1545, R628-F1545, H629-F1545, D630-F1545, C631-F1545, N632-F1545, F633-F1545, D634-F1545, K635-F1545, A636-F1545, M637-F1545, Q638-F1545, F639-F1545, S640-F1545, E641-F1545, A642-F1545, S643-F1545, F644-F1545, T645-F1545, W646-F1545, E647-F1545, H648-F1545, D649-F1545, S650-F1545, E651-F1545, A652-F1545, T653-F1545, V654-F1545, R655-F1545, D656-F1545, V657-F1545, N658-F1545, L659-F1545, D660-F1545, I661-F1545, M662-F1545, A663-F1545, G664-F1545, Q665-F1545, L666-F1545, V667-F1545, A668-F1545, V669-F1545, I670-F1545, G671-F1545, P672-F1545, V673-F1545, G674-F1545, S675-F1545, G676-F1545, K677-F1545, S678-F1545, S679-F1545, L680-F1545, I681-F1545, S682-F1545, A683-F1545, M684-F1545, L685-F1545, G686-F1545, E687-F1545, M688-F1545, E689-F1545, N690-F1545, V691-F1545, H692-F1545, G693-F1545, H694-F1545, I695-F1545, T696-F1545, I697-F1545, K698-F1545, G699-F1545, T700-F1545, T701-F1545, A702-F1545, Y703-F1545, V704-F1545, P705-F1545, Q706-F1545, Q707-F1545, S708-F1545, W709-F1545, I710-F1545, Q711-F1545, N712-F1545, G713-F1545, T714-F1545, I715-F1545, K716-F1545, D717-F1545, N718-F1545, I719-F1545, L720-F1545, F721-F1545, G722-F1545, T723-F1545, E724-F1545, F725-F1545, N726-F1545, E727-F1545, K728-F1545, R729-F1545, Y730-F1545, Q731-F1545, Q732-F1545, V733-F1545, L734-F1545, E735-F1545, A736-F1545, C737-F1545, A738-F1545, L739-F1545, L740-F1545, P741-F1545, D742-F1545, L743-F1545, E744-F1545, M745-F1545, L746-F1545, P747-F1545, G748-F1545, G749-F1545, D750-F1545, L751-F1545, A752-F1545, E753-F1545, I754-F1545, G755-F1545, E756-F1545, K757-F1545, G758-F1545, I759-F1545, N760-F1545, L761-F1545, S762-F1545, G763-F1545, G764-F1545, Q765-F1545, K766-F1545, Q767-F1545, R768-F1545, I769-F1545, S770-F1545, L771-F1545, A772-F1545, R773-F1545, A774-F1545, T775-F1545, Y776-F1545, Q777-F1545, N778-F1545, L779-F1545, D780-F1545, I781-F1545, Y782-F1545, L783-F1545, L784-F1545, D785-F1545, D786-F1545, P787-F1545, L788-F1545, S789-F1545, A790-F1545, V791-F1545, D792-F1545, A793-F1545, H794-F1545, V795-F1545, G796-F1545, K797-F1545, H798-F1545, I799-F1545, F800-F1545, N801-F1545, K802-F1545, V803-F1545, L804-F1545, G805-F1545, P806-F1545, N807-F1545, G808-F1545, L809-F1545, L810-F1545, K811-F1545, G812-F1545, K813-F1545, T814-F1545, R815-F1545, L816-F1545, L817-F1545, V818-F1545, T819-F1545, H820-F1545, S821-F1545, M822-F1545, H823-F1545, F824-F1545, L825-F1545, P826-F1545, Q827-F1545, V828-F1545, D829-F1545, E830-F1545, I831-F1545, V832-F1545, V833-F1545, L834-F1545, G835-F1545, N836-F1545, G837-F1545, T838-F1545, I839-F1545, V840-F1545, E841-F1545, K842-F1545, G843-F1545, S844-F1545, Y845-F1545, S846-F1545, A847-F1545, L848-F1545, L849-F1545, A850-F1545, K851-F1545, K852-F1545, G853-F1545, E854-F1545, F855-F1545, A856-F1545, K857-F1545, N858-F1545, L859-F1545, K860-F1545, T861-F1545, F862-F1545, L863-F1545, R864-F1545, H865-F1545, T866-F1545, G867-F1545, P868-F1545, E869-F1545, E870-F1545, E871-F1545, A872-F1545, T873-F1545, V874-F1545, H875-F1545, D876-F1545, G877-F1545, S878-F1545, E879-F1545, E880-F1545, E881-F1545, D882-F1545, D883-F1545, D884-F1545, Y885-F1545, G886-F1545, L887-F1545, I888-F1545, S889-F1545, S890-F1545, V891-F1545, E892-F1545, E893-F1545, I894-F1545, P895-F1545, E896-F1545, D897-F1545, A898-F1545, A899-F1545, S900-F1545, I901-F1545, T902-F1545, M903-F1545, R904-F1545, R905-F1545, E906-F1545, N907-F1545, S908-F1545, F909-F1545, R910-F1545, R911-F1545, T912-F1545, L913-F1545, S914-F1545, R915-F1545, S916-F1545, S917-F1545, R918-F1545, S919-F1545, N920-F1545, G921-F1545, R922-F1545, H923-F1545, L924-F1545, K925-F1545, S926-F1545, L927-F1545, R928-F1545, N929-F1545, S930-F1545, L931-F1545, K932-F1545, T933-F1545, R934-F1545, N935-F1545, V936-F1545, N937-F1545, S938-F1545, L939-F1545, K940-F1545, E941-F1545, D942-F1545, E943-F1545, E944-F1545, L945-F1545, V946-F1545, K947-F1545, G948-F1545, Q949-F1545, K950-F1545, L951-F1545, I952-F1545, K953-F1545, K954-F1545, E955-F1545, F956-F1545, I957-F1545, E958-F1545, T959-F1545, G960-F1545, and/or R961-F1545 of SEQ ID NO:38. Polynucleotide sequences encoding these polypeptides are also provided. The present invention also encompasses the use of these N-terminal cMOAT (SNP_ID:PS 101s11) deletion polypeptides as immunogenic and/or antigenic epitopes as described elsewhere herein.

In preferred embodiments, the following C-terminal cMOAT (SNP_ID:PS110s11) deletion polypeptides are encompassed by the present invention: M1-F1545, M1-K1544, M1-T1543, M1-S1542, M1-N1541, M1-V1540, M1-N1539, M1-E1538, M1-I1537, M1-G1536, M1-A1535, M1-E1534, M1-K1533, M1-A1532, M1-M1531, M1-F1530, M1-Y1529, M1-F1528, M1-P1527, M1-G1526, M1-P1525, M1-I1524, M1-Q1523, M1-L1522, M1-L1521, M1-E1520, M1-E1519, M1-P1518, M1-S1517, M1-G1516, M1-Y1515, M1-E1514, M1-I1513, M1-I1512, M1-K1511, M1-G1510, M1-N1509, M1-D1508, M1-L1507, M1-V1506, M1-M1505, M1-V1504, M1-K1503, M1-D1502, M1-S1501, M1-D1500, M1-M1499, M1-I1498, M1-T1497, M1-H1496, M1-L1495, M1-R1494, M1-H1493, M1-A1492, M1-I1491, M1-T1490, M1-I1489, M1-V1488, M1-T1487, M1-C1486, M1-H1485, M1-A1484, M1-F1483, M1-E1482, M1-N1481, M1-Q1480, M1-I1479, M1-T1478, M1-T1477, M1-Q1476, M1-I1475, M1-L1474, M1-N1473, M1-D1472, M1-T1471, M1-E1470, M1-L1469, M1-D1468, M1-V1467, M1-A1466, M1-A1465, M1-T1464, M1-A1463, M1-E1462, M1-D1461, M1-L1460, M1-V1459, M1-L1458, M1-I1457, M1-K1456, M1-S1455, M1-K1454, M1-R1453, M1-L1452, M1-L1451, M1-A1450, M1-R1449, M1-G1448, M1-L1447, M1-C1446, M1-L1445, M1-L1444, M1-Q1443, M1-R1442, M1-Q1441, M1-G1440, M1-I1439, M1-S1438, M1-L1437, M1-N1436, M1-G1435, M1-G1434, M1-A1433, M1-E1432, M1-T1431, M1-V1430, M1-E1429, M1-H1428, M1-S1427, M1-L1426, M1-G1425, M1-L1424, M1-Q1423, M1-L1422, M1-S1421, M1-A1420, M1-V1419, M1-F1418, M1-S1417, M1-K1416, M1-L1415, M1-H1414, M1-A1413, M1-L1412, M1-E1411, M1-L1410, M1-A1409, M1-K1408, M1-W1407, M1-I1406, M1-E1405, M1-E1404, M1-D1403, M1-S1402, M1-Y1401, M1-N1400, M1-N1399, M1-F1398, M1-P1397, M1-D1396, M1-L1395, M1-N1394, M1-M1393, M1-R1392, M1-L1391, M1-S1390, M1-G1389, M1-S1388, M1-F1387, M1-L1386, M1-I1385, M1-P1384, M1-D1383, M1-Q1382, M1-P1381, M1-I1380, M1-I1379, M1-T1378, M1-L1377, M1-K1376, M1-E1375, M1-R1374, M1-L1373, M1-D1372, M1-H1371, M1-L1370, M1-G1369, M1-I1368, M1-S1367, M1-A1366, M1-I1365, M1-D1364, M1-V1363, M1-G1362, M1-D1361, M1-I1360, M1-I1359, M1-I1358, M1-Q1357, M1-G1356, M1-G1355, M1-A1354, M1-A1353, M1-E1352, M1-L1351, M1-I1350, M1-R1349, M1-F1348, M1-L1347, M1-C1346, M1-N1345, M1-T1344, M1-L1343, M1-S1342, M1-S1341, M1-K1340, M1-G1339, M1-A1338, M1-G1337, M1-T1336, M1-R1335, M1-G1334, M1-V1333, M1-V1332, M1-G1331, M1-I1330, M1-K1329, M1-E1328, M1-M1327, M1-S1326, M1-G1325, M1-I1324, M1-D1323, M1-C1322, M1-T1321, M1-I1320, M1-G1319, M1-R1318, M1-L1317, M1-V1316, M1-L1315, M1-D1314, M1-L1313, M1-E1312, M1-P1311, M1-R1310, M1-Y1309, M1-R1308, M1-V1307, M1-Q1306, M1-Y1305, M1-N1304, M1-N1303, M1-F1302, M1-Q1301, M1-I1300, M1-K1299, M1-G1298, M1-K1297, M1-S1296, M1-P1295, M1-W1294, M1-D1293, M1-P1292, M1-P1291, M1-P1290, M1-R1289, M1-K1288, M1-D1287, M1-T1286, M1-V1285, M1-W1284, M1-P1283, M1-A1282, M1-E1281, M1-N1280, M1-E1279, M1-V1278, M1-K1277, M1-T1276, M1-Y1275, M1-E1274, M1-T1273, M1-I1272, M1-R1271, M1-E1270, M1-V1269, M1-A1268, M1-V1267, M1-I1266, M1-N1265, M1-T1264, M1-E1263, M1-I1262, M1-E1261, M1-S1260, M1-T1259, M1-M1258, M1-R1257, M1-V1256, M1-L1255, M1-W1254, M1-N1253, M1-L1252, M1-T1251, M1-Q1250, M1-T1249, M1-I1248, M1-N1247, M1-L1246, M1-A1245, M1-N1244, M1-S1243, M1-L1242, M1-V1241, M1-F1240, M1-G1239, M1-V1238, M1-T1237, M1-D1236, M1-G1235, M1-S1234, M1-L1233, M1-T1232, M1-D1231, M1-R1230, M1-Y1229, M1-I1228, M1-V1227, M1-M1226, M1-M1225, M1-L1224, M1-A1223, M1-S1222, M1-F1221, M1-F1220, M1-V1219, M1-T1218, M1-L1217, M1-N1216, M1-G1215, M1-V1214, M1-L1213, M1-E1212, M1-L1211, M1-R1210, M1-I1209, M1-A1208, M1-L1207, M1-W1206, M1-R1205, M1-N1204, M1-S1203, M1-T1202, M1-I1201, M1-W1200, M1-S1199, M1-F1198, M1-V1197, M1-C1196, M1-K1195, M1-Q1194, M1-N1193, M1-T1192, M1-D1191, M1-I1190, M1-R1189, M1-E1188, M1-E1187, M1-N1186, M1-H1185, M1-K1184, M1-L1183, M1-F1182, M1-R1181, M1-Q1180, M1-Q1179, M1-H1178, M1-E1177, M1-F1176, M1-A1175, M1-R1174, M1-I1173, M1-V1172, M1-P1171, M1-L1170, M1-G1169, M1-S1168, M1-V1167, M1-T1166, M1-E1165, M1-S1164, M1-F1163, M1-H1162, M1-S1161, M1-Y1160, M1-I1159, M1-P1158, M1-S1157, M1-R1156, M1-T1155, M1-V1154, M1-S1153, M1-D1152, M1-L1151, M1-R1150, M1-R1149, M1-L1148, M1-Q1147, M1-R1146, M1-S1145, M1-T1144, M1-S1143, M1-V1142, M1-Y1141, M1-F1140, M1-M1139, M1-Q1138, M1-V1137, M1-S1136, M1-V1135, M1-Y1134, M1-I1133, M1-I1132, M1-G1131, M1-L1130, M1-P1129, M1-I1128, M1-V1127, M1-I1126, M1-I1125, M1-T1124, M1-F1123, M1-V1122, M1-P1121, M1-T1120, M1-A1119, M1-M1118, M1-C1117, M1-I1116, M1-M1115, M1-V1114, M1-L1113, M1-T1112, M1-S1111, M1-I1110, M1-I1109, M1-G1108, M1-L1107, M1-F1106, M1-C1105, M1-T1104, M1-I1103, M1-W1102, M1-S1101, M1-R1100, M1-L1099, M1-S1098, M1-Q1097, M1-P1096, M1-L1095, M1-T1094, M1-D1093, M1-D1092, M1-V1091, M1-T1090, M1-S1089, M1-I1088, M1-D1087, M1-G1086, M1-A1085, M1-F1084, M1-R1083, M1-N1082, M1-V1081, M1-I1080, M1-R1079, M1-G1078, M1-T1077, M1-P1076, M1-T1075, M1-T1074, M1-D1073, M1-F1072, M1-F1071, M1-R1070, M1-M1069, M1-P1068, M1-A1067, M1-R1066, M1-L1065, M1-I1064, M1-N1063, M1-N1062, M1-L1061, M1-L1060, M1-Q1059, M1-K1058, M1-H1057, M1-L1056, M1-I1055, M1-N1054, M1-S1053, M1-A1052, M1-H1051, M1-V1050, M1-F1049, M1-G1048, M1-F1047, M1-A1046, M1-S1045, M1-W1044, M1-F1043, M1-H1042, M1-A1041, M1-I1040, M1-F1039, M1-V1038, M1-F1037, M1-I1036, M1-G1035, M1-Q1034, M1-A1033, M1-L1032, M1-G1031, M1-L1030, M1-A1029, M1-G1028, M1-Y1027, M1-V1026, M1-G1025, M1-V1024, M1-R1023, M1-M1022, M1-D1021, M1-R1020, M1-Q1019, M1-S1018, M1-A1017, M1-P1016, M1-Y1015, M1-D1014, M1-T1013, M1-S1012, M1-N1011, M1-F1010, M1-I1009, M1-K1008, M1-S1007, M1-D1006, M1-S1005, M1-T1004, M1-W1003, M1-A1002, M1-S1001, M1-L1000, M1-W999, M1-L998, M1-N997, M1-S996, M1-G995, M1-I994, M1-F993, M1-A992, M1-V991, M1-S990, M1-N989, M1-M988, M1-V987, M1-F986, M1-A985, M1-L984, M1-I983, M1-I982, M1-F981, M1-F980, M1-I979, M1-S978, M1-F977, M1-L976, M1-G975, M1-I974, M1-A973, M1-Q972, M1-L971, M1-Y970, M1-E969, M1-L968, M1-Y967, M1-I966, M1-S965, M1-F964, M1-K963, M1-V962, and/or M1-R961 of SEQ ID NO:38. Polynucleotide sequences encoding these polypeptides are also provided. The present invention also encompasses the use of these C-terminal cMOAT (SNP_ID:PS101s1) deletion polypeptides as immunogenic and/or antigenic epitopes as described elsewhere herein.

Alternatively, preferred polypeptides of the present invention may comprise polypeptide sequences corresponding to, for example, internal regions of the cMOAT (SNP_ID: PS101s11) polypeptide (e.g., any combination of both N- and C-terminal cMOAT (SNP_ID: PS101s11) polypeptide deletions) of SEQ ID NO:38. For example, internal regions could be defined by the equation: amino acid NX to amino acid CX, wherein NX refers to any N-terminal deletion polypeptide amino acid of cMOAT (SNP_ID: PS101s11) (SEQ ID NO:38), and where CX refers to any C-terminal deletion polypeptide amino acid of cMOAT (SNP_ID: PS101s11) (SEQ ID NO:38). Polynucleotides encoding these polypeptides are also provided. The present invention also encompasses the use of these polypeptides as an immunogenic and/or antigenic epitope as described elsewhere herein. Preferably, the resulting deletion polypeptide comprises the polypeptide polymorphic loci identified elsewhere herein for cMOAT (SNP_ID: PS101s11), and more preferably comprises the polypeptide polymorphic allele identified elsewhere herein for cMOAT (SNP_ID: PS 101s11).

### Features of the Polypeptide Encoded by Gene No: 18

The present invention relates to isolated nucleic acid molecules comprising, or alternatively, consisting of all or a portion of the variant allele of the human cMOAT, ATP-binding cassette sub-family C member 2 gene (SNP_ID: PS101s130) (e.g., wherein reference or wildtype cMOAT gene is exemplified by SEQ ID NO:1). Preferred portions are at least 10, preferably at least 20, preferably at least 40, preferably at least 100, contiguous polynucleotides and comprise a “G” at the nucleotide position corresponding to nucleotide 359 of the cMOAT gene, or a portion of SEQ ID NO:39. Alternatively, preferred portions are at least 10, preferably at least 20, preferably at least 40, preferably at least 100, contiguous polynucleotides and comprise an “A” at the nucleotide position corresponding to nucleotide 359 of the cMOAT gene, or a portion of SEQ ID NO:39. The invention further relates to isolated gene products, e.g., polypeptides and/or proteins, which are encoded by a nucleic acid molecule comprising all or a portion of the variant allele of the cMOAT gene.

In one embodiment, the invention relates to a method for predicting the likelihood that an individual will have a disorder associated with a “G” at the nucleotide position corresponding to nucleotide position 359 of SEQ ID NO:39 (or diagnosing or aiding in the diagnosis of such a disorder) comprising the steps of obtaining a DNA sample from an individual to be assessed and determining the nucleotide present at position 359 of SEQ ID NO:39. The presence of a “G” at this position indicates that the individual has a greater likelihood of having a disorder associated therewith than an individual having an “A” at that position, or a greater likelihood of having more severe symptoms.

Conversely, the invention relates to a method for predicting the likelihood that an individual will have a disorder associated with an “A” at the nucleotide position corresponding to nucleotide position 359 of SEQ ID NO:39 (or diagnosing or aiding in the diagnosis of such a disorder) comprising the steps of obtaining a DNA sample from an individual to be assessed and determining the nucleotide present at position 359 of SEQ ID NO:39. The presence of an “A” at this position indicates that the individual has a greater likelihood of having a disorder associated therewith than an individual having a “G” at that position, or a greater likelihood of having more severe symptoms.

Representative disorders which may be detected, diagnosed, identified, treated, prevented, and/or ameliorated by the present invention include, the following, non-limiting diseases and disorders: decreased heptic uptake of administered statins, resistance to beneficial responses of administered statins; resistance to LDL lowering responses to administered statins; resistance to TG lowering responses to administered statins; resistance to HDL elevating responses to administered statins; increased incidence of drug interactions between one or more administered statins; increased incidence of drug and endogenous substance interactions between a statin drug and endogenous substrates of cMOAT including cholate, taurocholate, thyroid hormones T3 and T4, DHEAS, estradiol-17beta-glucuronide, estrone-3-sulfate, prostaglandin E2, thromboxane B2, leukotriene C4, leukotriene E4, and bilirubin and its conjugates; decreased response to HMG-CoA reductase inhibitors; in addition to other disorders referenced herein, which include, for example, any disorder related to high levels of circulating LDL, high levels of TG, and/or low levels of circularing HDL, which include, but are not limited to, cardiovascular diseases, hepatic diseases, angina pectoris, hypertension, heart failure, myocardial infarction, ventricular hypertrophy, vascular diseases, miscrovascular disease, vascular leak syndrome, aneurysm, stroke, embolism, thrombosis, endothelial dysfunction, coronary artery disease, arteriosclerosis, and/or atherosclerosis.

### Features of the Polypeptide Encoded by Gene No:19

The present invention relates to isolated nucleic acid molecules comprising, or alternatively, consisting of all or a portion of the variant allele of the human cMOAT, ATP-binding cassette sub-family C member 2 gene (SNP_ID: PS101s220) (e.g., wherein reference or wildtype cMOAT gene is exemplified by SEQ ID NO:1). Preferred portions are at least 10, preferably at least 20, preferably at least 40, preferably at least 100, contiguous polynucleotides and comprise a “C” at the nucleotide position corresponding to nucleotide 2110 of the cMOAT gene, or a portion of SEQ ID NO:41. Alternatively, preferred portions are at least 10, preferably at least 20, preferably at least 40, preferably at least 100, contiguous polynucleotides and comprise a “T” at the nucleotide position corresponding to nucleotide 2110 of the cMOAT gene, or a portion of SEQ ID NO:41. The invention further relates to isolated gene products, e.g., polypeptides and/or proteins, which are encoded by a nucleic acid molecule comprising all or a portion of the variant allele of the cMOAT gene.

In one embodiment, the invention relates to a method for predicting the likelihood that an individual will have a disorder associated with a “C” at the nucleotide position corresponding to nucleotide position 2110 of SEQ ID NO:41 (or diagnosing or aiding in the diagnosis of such a disorder) comprising the steps of obtaining a DNA sample from an individual to be assessed and determining the nucleotide present at position 2110 of SEQ ID NO:41. The presence of a “C” at this position indicates that the individual has a greater likelihood of having a disorder associated therewith than an individual having a “T” at that position, or a greater likelihood of having more severe symptoms.

Conversely, the invention relates to a method for predicting the likelihood that an individual will have a disorder associated with a “T” at the nucleotide position corresponding to nucleotide position 2110 of SEQ ID NO:41 (or diagnosing or aiding in the diagnosis of such a disorder) comprising the steps of obtaining a DNA sample from an individual to be assessed and determining the nucleotide present at position 2110 of SEQ ID NO:41. The presence of a “T” at this position indicates that the individual has a greater likelihood of having a disorder associated therewith than an individual having a “C” at that position, or a greater likelihood of having more severe symptoms.

The present invention further relates to isolated proteins or polypeptides comprising, or alternatively, consisting of all or a portion of the encoded variant amino acid sequence of the human human cMOAT, solute carrier family 21 member 6 polypeptide (e.g., wherein reference or wildtype human cMOAT, solute carrier family 21 member 6 polypeptide is exemplified by SEQ ID NO:2). Preferred portions are at least 10, preferably at least 20, preferably at least 40, preferably at least 100, contiguous polypeptides and comprises a “T” at the amino acid position corresponding to amino acid 670 of the cMOAT polypeptide, or a portion of SEQ ID NO:42. Alternatively, preferred portions are at least 10, preferably at least 20, preferably at least 40, preferably at least 100, contiguous polypeptides and comprises a “I” at the amino acid position corresponding to amino acid 670 of the cMOAT protein, or a portion of SEQ ID NO:42. The invention further relates to isolated nucleic acid molecules encoding such polypeptides or proteins, as well as to antibodies that bind to such proteins or polypeptides.

Representative disorders which may be detected, diagnosed, identified, treated, prevented, and/or ameliorated by the present invention include, the following, non-limiting diseases and disorders: decreased heptic uptake of administered statins, resistance to beneficial responses of administered statins; resistance to LDL lowering responses to administered statins; resistance to TG lowering responses to administered statins; resistance to HDL elevating responses to administered statins; increased incidence of drug interactions between one or more administered statins; increased incidence of drug and endogenous substance interactions between a statin drug and endogenous substrates of cMOAT including cholate, taurocholate, thyroid hormones T3 and T4, DHEAS, estradiol-17beta-glucuronide, estrone-3-sulfate, prostaglandin E2, thromboxane B2, leukotriene C4, leukotriene E4, and bilirubin and its conjugates; decreased response to HMG-CoA reductase inhibitors; in addition to other disorders referenced herein, which include, for example, any disorder related to high levels of circulating LDL, high levels of TG, and/or low levels of circularing HDL, which include, but are not limited to, cardiovascular diseases, hepatic diseases, angina pectoris, hypertension, heart failure, myocardial infarction, ventricular hypertrophy, vascular diseases, miscrovascular disease, vascular leak syndrome, aneurysm, stroke, embolism, thrombosis, endothelial dysfunction, coronary artery disease, arteriosclerosis, and/or atherosclerosis.

In preferred embodiments, the following N-terminal cMOAT (SNP_ID:PS101s22) deletion polypeptides are encompassed by the present invention: M1-F1545, L2-F1545, E3-F1545, K4-F1545, F5-F1545, C6-F1545, N7-F1545, S8-F1545, T9-F1545, F10-F1545, W11-F1545, N12-F1545, S13-F1545, S14-F1545, F15-F1545, L16-F1545, D17-F1545, S18-F1545, P19-F1545, E20-F1545, A21-F1545, D22-F1545, L23-F1545, P24-F1545, L25-F1545, C26-F1545, F27-F1545, E28-F1545, Q29-F1545, T30-F1545, V31-F1545, L32-F1545, V33-F1545, W34-F1545, I35-F1545, P36-F1545, L37-F1545, G38-F1545, F39-F1545, L40-F1545, W41-F1545, LA2-F1545, L43-F1545, A44-F1545, P45-F1545, W46-F1545, Q47-F1545, L48-F1545, L49-F1545, H50-F1545, V51-F1545, Y52-F1545, K53-F1545, S54-F1545, R55-F1545, T56-F1545, K57-F1545, R58-F1545, S59-F1545, S60-F1545, T61-F1545, T62-F1545, K63-F1545, L64-F1545, Y65-F1545, L66-F1545, A67-F1545, K68-F1545, Q69-F1545, V70-F1545, F71-F1545, V72-F1545, G73-F1545, F74-F1545, L75-F1545, L76-F1545, I77-F1545, L78-F1545, A79-F1545, A80-F1545, I81-F1545, E82-F1545, L83-F1545, A84-F1545, L85-F1545, V86-F1545, L87-F1545, T88-F1545, E89-F1545, D90-F1545, S91-F1545, G92-F1545, Q93-F1545, A94-F1545, T95-F1545, V96-F1545, P97-F1545, A98-F1545, V99-F1545, R100-F1545, Y101-F1545, T102-F1545, N103-F1545, P104-F1545, S105-F1545, L106-F1545, Y107-F1545, L108-F1545, G109-F1545, T110-F1545, W111-F1545, L112-F1545, L113-F1545, V114-F1545, L115-F1545, L116-F1545, I117-F1545, Q118-F1545, Y119-F1545, S120-F1545, R121-F1545, Q122-F1545, W123-F1545, C124-F1545, V125-F1545, Q126-F1545, K127-F1545, N128-F1545, S129-F1545, W130-F1545, F131-F1545, L132-F1545, S133-F1545, L134-F1545, F135-F1545, W136-F1545, I137-F1545, L138-F1545, S139-F1545, I140-F1545, L141-F1545, C142-F1545, G143-F1545, T144-F1545, F145-F1545, Q146-F1545, F147-F1545, Q148-F1545, T149-F1545, L150-F1545, I151-F1545, R152-F1545, T153-F1545, L154-F1545, L155-F1545, Q156-F1545, G157-F1545, D158-F1545, N159-F1545, S160-F1545, N161-F1545, L162-F1545, A163-F1545, Y164-F1545, S165-F1545, C166-F1545, L167-F1545, F168-F1545, F169-F1545, I170-F1545, S171-F1545, Y172-F1545, G173-F1545, F174-F1545, Q175-F1545, I176-F1545, L177-F1545, I178-F1545, L179-F1545, I180-F1545, F181-F1545, S182-F1545, A183-F1545, F184-F1545, S185-F1545, E186-F1545, N187-F1545, N188-F1545, E189-F1545, S190-F1545, S191-F1545, N192-F1545, N193-F1545, P194-F1545, S195-F1545, S196-F1545, I197-F1545, A198-F1545, S199-F1545, F200-F1545, L201-F1545, S202-F1545, S203-F1545, I204-F1545, T205-F1545, Y206-F1545, S207-F1545, W208-F1545, Y209-F1545, D210-F1545, S211-F1545, I212-F1545, I213-F1545, L214-F1545, K215-F1545, G216-F1545, Y217-F1545, K218-F1545, R219-F1545, P220-F1545, L221-F1545, T222-F1545, L223-F1545, E224-F1545, D225-F1545, V226-F1545, W227-F1545, E228-F1545, V229-F1545, D230-F1545, E231-F1545, E232-F1545, M233-F1545, K234-F1545, T235-F1545, K236-F1545, T237-F1545, L238-F1545, V239-F1545, S240-F1545, K241-F1545, F242-F1545, E243-F1545, T244-F1545, H245-F1545, M246-F1545, K247-F1545, R248-F1545, E249-F1545, L250-F1545, Q251-F1545, K252-F1545, A253-F1545, R254-F1545, R255-F1545, A256-F1545, L257-F1545, Q258-F1545, R259-F1545, R260-F1545, Q261-F1545, E262-F1545, K263-F1545, S264-F1545, S265-F1545, Q266-F1545, Q267-F1545, N268-F1545, S269-F1545, G270-F1545, A271-F1545, R272-F1545, L273-F1545, P274-F1545, G275-F1545, L276-F1545, N277-F1545, K278-F1545, N279-F1545, Q280-F1545, S281-F1545, Q282-F1545, S283-F1545, Q284-F1545, D285-F1545, A286-F1545, L287-F1545, V288-F1545, L289-F1545, E290-F1545, D291-F1545, V292-F1545, E293-F1545, K294-F1545, K295-F1545, K296-F1545, K297-F1545, K298-F1545, S299-F1545, G300-F1545, T301-F1545, K302-F1545, K303-F1545, D304-F1545, V305-F1545, P306-F1545, K307-F1545, S308-F1545, W309-F1545, L310-F1545, M311-F1545, K312-F1545, A313-F1545, L314-F1545, F315-F1545, K316-F1545, T317-F1545, F318-F1545, Y319-F1545, M320-F1545, V321-F1545, L322-F1545, L323-F1545, K324-F1545, S325-F1545, F326-F1545, L327-F1545, L328-F1545, K329-F1545, L330-F1545, V331-F1545, N332-F1545, D333-F1545, I334-F1545, F335-F1545, T336-F1545, F337-F1545, V338-F1545, S339-F1545, P340-F1545, Q341-F1545, L342-F1545, L343-F1545, K344-F1545, L345-F1545, L346-F1545, I347-F1545, S348-F1545, F349-F1545, A350-F1545, S351-F1545, D352-F1545, R353-F1545, D354-F1545, T355-F1545, Y356-F1545, L357-F1545, W358-F1545, I359-F1545, G360-F1545, Y361-F1545, L362-F1545, C363-F1545, A364-F1545, I365-F1545, L366-F1545, L367-F1545, F368-F1545, T369-F1545, A370-F1545, A371-F1545, L372-F1545, I373-F1545, Q374-F1545, S375-F1545, F376-F1545, C377-F1545, L378-F1545, Q379-F1545, C380-F1545, Y381-F1545, F382-F1545, Q383-F1545, L384-F1545, C385-F1545, F386-F1545, K387-F1545, L388-F1545, G389-F1545, V390-F1545, K391-F1545, V392-F1545, R393-F1545, T394-F1545, A395-F1545, I396-F1545, M397-F1545, A398-F1545, S399-F1545, V400-F1545, Y401-F1545, K402-F1545, K403-F1545, A404-F1545, L405-F1545, T406-F1545, L407-F1545, S408-F1545, N409-F1545, L410-F1545, A411-F1545, R412-F1545, K413-F1545, E414-F1545, Y415-F1545, T416-F1545, V417-F1545, G418-F1545, E419-F1545, T420-F1545, V421-F1545, N422-F1545, L423-F1545, M424-F1545, S425-F1545, V426-F1545, D427-F1545, A428-F1545, Q429-F1545, K430-F1545, L431-F1545, M432-F1545, D433-F1545, V434-F1545, T435-F1545, N436-F1545, F437-F1545, M438-F1545, H439-F1545, M440-F1545, L441-F1545, W442-F1545, S443-F1545, S444-F1545, V445-F1545, L446-F1545, Q447-F1545, I448-F1545, V449-F1545, L450-F1545, S451-F1545, I452-F1545, F453-F1545, F454-F1545, L455-F1545, W456-F1545, R457-F1545, E458-F1545, L459-F1545, G460-F1545, P461-F1545, S462-F1545, V463-F1545, L464-F1545, A465-F1545, G466-F1545, V467-F1545, G468-F1545, V469-F1545, M470-F1545, V471-F1545, L472-F1545, V473-F1545, I474-F1545, P475-F1545, I476-F1545, N477-F1545, A478-F1545, I479-F1545, L480-F1545, S481-F1545, T482-F1545, K483-F1545, S484-F1545, K485-F1545, T486-F1545, I487-F1545, Q488-F1545, V489-F1545, K490-F1545, N491-F1545, M492-F1545, K493-F1545, N494-F1545, K495-F1545, D496-F1545, K497-F1545, R498-F1545, L499-F1545, K500-F1545, I501-F1545, M502-F1545, N503-F1545, E504-F1545, I505-F1545, L506-F1545, S507-F1545, G508-F1545, I509-F1545, K510-F1545, I511-F1545, L512-F1545, K513-F1545, Y514-F1545, F515-F1545, A516-F1545, W517-F1545, E518-F1545, P519-F1545, S520-F1545, F521-F1545, R522-F1545, D523-F1545, Q524-F1545, V525-F1545, Q526-F1545, N527-F1545, L528-F1545, R529-F1545, K530-F1545, K531-F1545, E532-F1545, L533-F1545, K534-F1545, N535-F1545, L536-F1545, L537-F1545, A538-F1545, F539-F1545, S540-F1545, Q541-F1545, L542-F1545, Q543-F1545, C544-F1545, V545-F1545, V546-F1545, I547-F1545, F548-F1545, V549-F1545, F550-F1545, Q551-F1545, L552-F1545, T553-F1545, P554-F1545, V555-F1545, L556-F1545, V557-F1545, S558-F1545, V559-F1545, V560-F1545, T561-F1545, F562-F1545, S563-F1545, V564-F1545, Y565-F1545, V566-F1545, L567-F1545, V568-F1545, D569-F1545, S570-F1545, N571-F1545, N572-F1545, I573-F1545, L574-F1545, D575-F1545, A576-F1545, Q577-F1545, K578-F1545, A579-F1545, F580-F1545, T581-F1545, S582-F1545, I583-F1545, T584-F1545, L585-F1545, F586-F1545, N587-F1545, I588-F1545, L589-F1545, R590-F1545, F591-F1545, P592-F1545, L593-F1545, S594-F1545, M595-F1545, L596-F1545, P597-F1545, M598-F1545, M599-F1545, I600-F1545, S601-F1545, S602-F1545, M603-F1545, L604-F1545, Q605-F1545, A606-F1545, S607-F1545, V608-F1545, S609-F1545, T610-F1545, E611-F1545, R612-F1545, L613-F1545, E614-F1545, K615-F1545, Y616-F1545, L617-F1545, G618-F1545, G619-F1545, D620-F1545, D621-F1545, L622-F1545, D623-F1545, T624-F1545, S625-F1545, A626-F1545, I627-F1545, R628-F1545, H629-F1545, D630-F1545, C631-F1545, N632-F1545, F633-F1545, D634-F1545, K635-F1545, A636-F1545, M637-F1545, Q638-F1545, F639-F1545, S640-F1545, E641-F1545, A642-F1545, S643-F1545, F644-F1545, T645-F1545, W646-F1545, E647-F1545, H648-F1545, D649-F1545, S650-F1545, E651-F1545, A652-F1545, T653-F1545, V654-F1545, R655-F1545, D656-F1545, V657-F1545, N658-F1545, L659-F1545, D660-F1545, I661-F1545, M662-F1545, A663-F1545, G664-F1545, Q665-F1545, L666-F1545, V667-F1545, A668-F1545, V669-F1545, and/or T670-F1545 of SEQ ID NO:42. Polynucleotide sequences encoding these polypeptides are also provided. The present invention also encompasses the use of these N-terminal cMOAT (SNP_ID:PS101s22) deletion polypeptides as immunogenic and/or antigenic epitopes as described elsewhere herein.

In preferred embodiments, the following C-terminal cMOAT (SNP_ID:PS101s22) deletion polypeptides are encompassed by the present invention: M1-F1545, M1-K1544, M1-T1543, M1-S1542, M1-N1541, M1-V1540, M1-N1539, M1-E1538, M1-I1537, M1-G1536, M1-A1535, M1-E1534, M1-K1533, M1-A1532, M1-M1531, M1-F1530, M1-Y1529, M1-F1528, M1-P1527, M1-G1526, M1-P1525, M1-I1524, M1-Q1523, M1-L1522, M1-L1521, M1-E1520, M1-E1519, M1-P1518, M1-S1517, M1-G1516, M1-Y1515, M1-E1514, M1-I1513, M1-I1512, M1-K1511, M1-G1510, M1-N1509, M1-D1508, M1-L1507, M1-V1506, M1-M1505, M1-V1504, M1-K1503, M1-D1502, M1-S1501, M1-D1500, M1-M1499, M1-I1498, M1-T1497, M1-H1496, M1-L1495, M1-R1494, M1-H1493, M1-A1492, M1-I1491, M1-T1490, M1-I1489, M1-V1488, M1-T1487, M1-C1486, M1-H1485, M1-A1484, M1-F1483, M1-E1482, M1-N1481, M1-Q1480, M1-I1479, M1-T1478, M1-T1477, M1-Q1476, M1-I1475, M1-L1474, M1-N1473, M1-D1472, M1-T1471, M1-E1470, M1-L1469, M1-D1468, M1-V1467, M1-A1466, M1-A1465, M1-T1464, M1-A1463, M1-E1462, M1-D1461, M1-L1460, M1-V1459, M1-L1458, M1-I1457, M1-K1456, M1-S1455, M1-K1454, M1-R1453, M1-L1452, M1-L1451, M1-A1450, M1-R1449, M1-G1448, M1-L1447, M1-C1446, M1-L1445, M1-L1444, M1-Q1443, M1-R1442, M1-Q1441, M1-G1440, M1-I1439, M1-S1438, M1-L1437, M1-N1436, M1-G1435, M1-G1434, M1-A1433, M1-E1432, M1-T1431, M1-V1430, M1-E1429, M1-H1428, M1-S1427, M1-L1426, M1-G1425, M1-L1424, M1-Q1423, M1-L1422, M1-S1421, M1-A1420, M1-V1419, M1-F1418, M1-S1417, M1-K1416, M1-L1415, M1-H1414, M1-A1413, M1-L1412, M1-E1411, M1-L1410, M1-A1409, M1-K1408, M1-W1407, M1-I1406, M1-E1405, M1-E1404, M1-D1403, M1-S1402, M1-Y1401, M1-N1400, M1-N1399, M1-F1398, M1-P1397, M1-D1396, M1-L1395, M1-N1394, M1-M1393, M1-R1392, M1-L1391, M1-S1390, M1-G1389, M1-S1388, M1-F1387, M1-L1386, M1-I1385, M1-P1384, M1-D1383, M1-Q1382, M1-P1381, M1-I1380, M1-I1379, M1-T1378, M1-L1377, M1-K1376, M1-E1375, M1-R1374, M1-L1373, M1-D1372, M1-H1371, M1-L1370, M1-G1369, M1-I1368, M1-S1367, M1-A1366, M1-I1365, M1-D1364, M1-V1363, M1-G1362, M1-D1361, M1-I1360, M1-I1359, M1-I1358, M1-Q1357, M1-G1356, M1-G1355, M1-A1354, M1-A1353, M1-E1352, M1-L1351, M1-I1350, M1-R1349, M1-F1348, M1-L1347, M1-C1346, M1-N1345, M1-T1344, M1-L1343, M1-S1342, M1-S1341, M1-K1340, M1-G1339, M1-A1338, M1-G1337, M1-T1336, M1-R1335, M1-G1334, M1-V1333, M1-V1332, M1-G1331, M1-I1330, M1-K1329, M1-E1328, M1-M1327, M1-S1326, M1-G1325, M1-I1324, M1-D1323, M1-C1322, M1-T1321, M1-I1320, M1-G1319, M1-R1318, M1-L1317, M1-V1316, M1-L1315, M1-D1314, M1-L1313, M1-E1312, M1-P1311, M1-R1310, M1-Y1309, M1-R1308, M1-V1307, M1-Q1306, M1-Y1305, M1-N1304, M1-N1303, M1-F1302, M1-Q1301, M1-I1300, M1-K1299, M1-G1298, M1-K1297, M1-S1296, M1-P1295, M1-W1294, M1-D1293, M1-P1292, M1-P1291, M1-P1290, M1-R1289, M1-K1288, M1-D1287, M1-T1286, M1-V1285, M1-W1284, M1-P1283, M1-A1282, M1-E1281, M1-N1280, M1-E1279, M1-V1278, M1-K1277, M1-T1276, M1-Y1275, M1-E1274, M1-T1273, M1-I1272, M1-R1271, M1-E1270, M1-V1269, M1-A1268, M1-V1267, M1-I1266, M1-N1265, M1-T1264, M1-E1263, M1-I1262, M1-E1261, M1-S1260, M1-T1259, M1-M1258, M1-R1257, M1-V1256, M1-L1255, M1-W1254, M1-N1253, M1-L1252, M1-T1251, M1-Q1250, M1-T1249, M1-I1248, M1-N1247, M1-L1246, M1-A1245, M1-N1244, M1-S1243, M1-L1242, M1-V1241, M1-F1240, M1-G1239, M1-V1238, M1-T1237, M1-D1236, M1-G1235, M1-S1234, M1-L1233, M1-T1232, M1-D1231, M1-R1230, M1-Y1229, M1-I1228, M1-V1227, M1-M1226, M1-M1225, M1-L1224, M1-A1223, M1-S1222, M1-F1221, M1-F1220, M1-V1219, M1-T1218, M1-L1217, M1-N1216, M1-G1215, M1-V1214, M1-L1213, M1-E1212, M1-L1211, M1-R1210, M1-I1209, M1-A1208, M1-L1207, M1-W1206, M1-R1205, M1-N1204, M1-S1203, M1-T1202, M1-I1201, M1-W1200, M1-S1199, M1-F1198, M1-V1197, M1-C1196, M1-K1195, M1-Q1194, M1-N1193, M1-T1192, M1-D1191, M1-I1190, M1-R1189, M1-E1188, M1-E1187, M1-N1186, M1-H1185, M1-K1184, M1-L1183, M1-F1182, M1-R1181, M1-Q1180, M1-Q1179, M1-H1178, M1-E1177, M1-F1176, M1-A1175, M1-R1174, M1-I1173, M1-V1172, M1-P1171, M1-L1170, M1-G1169, M1-S1168, M1-V1167, M1-T1166, M1-E1165, M1-S1164, M1-F1163, M1-H1162, M1-S1161, M1-Y1160, M1-I1159, M1-P1158, M1-S1157, M1-R1156, M1-T1155, M1-V1154, M1-S1153, M1-D1152, M1-L1151, M1-R1150, M1-R1149, M1-L1148, M1-Q1147, M1-R1146, M1-S1145, M1-T1144, M1-S1143, M1-V1142, M1-Y1141, M1-F1140, M1-M1139, M1-Q1138, M1-V1137, M1-S1136, M1-V1135, M1-Y1134, M1-I1133, M1-I1132, M1-G1131, M1-L1130, M1-P1129, M1-I1128, M1-V1127, M1-I1126, M1-I1125, M1-T1124, M1-F1123, M1-V1122, M1-P1121, M1-T1120, M1-A1119, M1-M1118, M1-C1117, M1-I1116, M1-M1115, M1-V1114, M1-L1113, M1-T1112, M1-S1111, M1-I1110, M1-I1109, M1-G1108, M1-L1107, M1-F1106, M1-C1105, M1-T1104, M1-I1103, M1-W1102, M1-S1101, M1-R1100, M1-L1099, M1-S1098, M1-Q1097, M1-P1096, M1-L1095, M1-T1094, M1-D1093, M1-D1092, M1-V1091, M1-T1090, M1-S1089, M1-I1088, M1-D1087, M1-G1086, M1-A1085, M1-F1084, M1-R1083, M1-N1082, M1-V1081, M1-I1080, M1-R1079, M1-G1078, M1-T1077, M1-P1076, M1-T1075, M1-T1074, M1-D1073, M1-F1072, M1-F1071, M1-R1070, M1-M1069, M1-P1068, M1-A1067, M1-R1066, M1-L1065, M1-I1064, M1-N1063, M1-N1062, M1-L1061, M1-L1060, M1-Q1059, M1-K1058, M1-H1057, M1-L1056, M1-I1055, M1-N1054, M1-S1053, M1-A1052, M1-H1051, M1-V1050, M1-F1049, M1-G1048, M1-F1047, M1-A1046, M1-S1045, M1-W1044, M1-F1043, M1-H1042, M1-A1041, M1-I1040, M1-F1039, M1-V1038, M1-F1037, M1-I1036, M1-G1035, M1-Q1034, M1-A1033, M1-L1032, M1-G1031, M1-L1030, M1-A1029, M1-G1028, M1-Y1027, M1-V1026, M1-G1025, M1-V1024, M1-R1023, M1-M1022, M1-D1021, M1-R1020, M1-Q1019, M1-S1018, M1-A1017, M1-P1016, M1-Y1015, M1-D1014, M1-T1013, M1-S1012, M1-N1011, M1-F1010, M1-I1009, M1-K1008, M1-S1007, M1-D1006, M1-S1005, M1-T1004, M1-W1003, M1-A1002, M1-S1001, M1-L1000, M1-W999, M1-L998, M1-N997, M1-S996, M1-G995, M1-I994, M1-F993, M1-A992, M1-V991, M1-S990, M1-N989, M1-M988, M1-V987, M1-F986, M1-A985, M1-L984, M1-I983, M1-I982, M1-F981, M1-F980, M1-I979, M1-S978, M1-F977, M1-L976, M1-G975, M1-I974, M1-A973, M1-Q972, M1-L971, M1-Y970, M1-E969, M1-L968, M1-Y967, M1-I966, M1-S965, M1-F964, M1-K963, M1-V962, M1-K961, M1-G960, M1-T959, M1-E958, M1-I957, M1-F956, M1-E955, M1-K954, M1-K953, M1-I952, M1-L951, M1-K950, M1-Q949, M1-G948, M1-K947, M1-V946, M1-L945, M1-E944, M1-E943, M1-D942, M1-E941, M1-K940, M1-L939, M1-S938, M1-N937, M1-V936, M1-N935, M1-R934, M1-T933, M1-K932, M1-L931, M1-S930, M1-N929, M1-R928, M1-L927, M1-S926, M1-K925, M1-L924, M1-H923, M1-R922, M1-G921, M1-N920, M1-S919, M1-R918, M1-S917, M1-S916, M1-R915, M1-S914, M1-L913, M1-T912, M1-R911, M1-R910, M1-F909, M1-S908, M1-N907, M1-E906, M1-R905, M1-R904, M1-M903, M1-T902, M1-I901, M1-S900, M1-A899, M1-A898, M1-D897, M1-E896, M1-P895, M1-I894, M1-E893, M1-E892, M1-V891, M1-S890, M1-S889, M1-I888, M1-L887, M1-G886, M1-Y885, M1-D884, M1-D883, M1-D882, M1-E881, M1-E880, M1-E879, M1-S878, M1-G877, M1-D876, M1-H875, M1-V874, M1-T873, M1-A872, M1-E871, M1-E870, M1-E869, M1-P868, M1-G867, M1-T866, M1-H865, M1-R864, M1-L863, M1-F862, M1-T861, M1-K860, M1-L859, M1-N858, M1-K857, M1-A856, M1-F855, M1-E854, M1-G853, M1-K852, M1-K851, M1-A850, M1-L849, M1-L848, M1-A847, M1-S846, M1-Y845, M1-S844, M1-G843, M1-K842, M1-E841, M1-V840, M1-I839, M1-T838, M1-G837, M1-N836, M1-G835, M1-L834, M1-V833, M1-V832, M1-I831, M1-E830, M1-D829, M1-V828, M1-Q827, M1-P826, M1-L825, M1-F824, M1-H823, M1-M822, M1-S821, M1-H820, M1-T819, M1-V818, M1-L817, M1-L816, M1-R815, M1-T814, M1-K813, M1-G812, M1-K811, M1-L810, M1-L809, M1-G808, M1-N807, M1-P806, M1-G805, M1-L804, M1-V803, M1-K802, M1-N801, M1-F800, M1-I799, M1-H798, M1-K797, M1-G796, M1-V795, M1-H794, M1-A793, M1-D792, M1-V791, M1-A790, M1-S789, M1-L788, M1-P787, M1-D786, M1-D785, M1-L784, M1-L783, M1-Y782, M1-I781, M1-D780, M1-L779, M1-N778, M1-Q777, M1-Y776, M1-T775, M1-A774, M1-R773, M1-A772, M1-L771, M1-S770, M1-I769, M1-R768, M1-Q767, M1-K766, M1-Q765, M1-G764, M1-G763, M1-S762, M1-L761, M1-N760, M1-I759, M1-G758, M1-K757, M1-E756, M1-G755, M1-I754, M1-E753, M1-A752, M1-L751, M1-D750, M1-G749, M1-G748, M1-P747, M1-L746, M1-M745, M1-E744, M1-L743, M1-D742, M1-P741, M1-L740, M1-L739, M1-A738, M1-C737, M1-A736, M1-E735, M1-L734, M1-V733, M1-Q732, M1-Q731, M1-Y730, M1-R729, M1-K728, M1-E727, M1-N726, M1-F725, M1-E724, M1-T723, M1-G722, M1-F721, M1-L720, M1-I719, M1-N718, M1-D717, M1-K716, M1-I715, M1-T714, M1-G713, M1-N712, M1-Q711, M1-I710, M1-W709, M1-S708, M1-Q707, M1-Q706, M1-P705, M1-V704, M1-Y703, M1-A702, M1-T701, M1-T700, M1-G699, M1-K698, M1-I697, M1-T696, M1-I695, M1-H694, M1-G693, M1-H692, M1-V691, M1-N690, M1-E689, M1-M688, M1-E687, M1-G686, M1-L685, M1-M684, M1-A683, M1-S682, M1-I681, M1-L680, M1-S679, M1-S678, M1-K677, M1-G676, M1-S675, M1-G674, M1-V673, M1-P672, M1-G671, and/or M1-T670 of SEQ ID NO:42. Polynucleotide sequences encoding these polypeptides are also provided. The present invention also encompasses the use of these C-terminal cMOAT (SNP_ID:PS101s22) deletion polypeptides as immunogenic and/or antigenic epitopes as described elsewhere herein.

Alternatively, preferred polypeptides of the present invention may comprise polypeptide sequences corresponding to, for example, internal regions of the cMOAT (SNP_ID: PS101s22) polypeptide (e.g., any combination of both N- and C-terminal cMOAT (SNP_ID: PS101s22) polypeptide deletions) of SEQ ID NO:42. For example, internal regions could be defined by the equation: amino acid NX to amino acid CX, wherein NX refers to any N-terminal deletion polypeptide amino acid of cMOAT (SNP_ID: PS101s22) (SEQ ID NO:42), and where CX refers to any C-terminal deletion polypeptide amino acid of cMOAT (SNP_ID: PS101s22) (SEQ ID NO:42). Polynucleotides encoding these polypeptides are also provided. The present invention also encompasses the use of these polypeptides as an immunogenic and/or antigenic epitope as described elsewhere herein. Preferably, the resulting deletion polypeptide comprises the polypeptide polymorphic loci identified elsewhere herein for cMOAT (SNP_ID: PS101s22), and more preferably comprises the polypeptide polymorphic allele identified elsewhere herein for cMOAT (SNP_ID: PS101s22).

### Features of the Polypeptide Encoded by Gene No:20

The present invention relates to isolated nucleic acid molecules comprising, or alternatively, consisting of all or a portion of the variant allele of the human cMOAT, ATP-binding cassette sub-family C member 2 gene (SNP_ID: PS101s230) (e.g., wherein reference or wildtype cMOAT gene is exemplified by SEQ ID NO:1). Preferred portions are at least 10, preferably at least 20, preferably at least 40, preferably at least 100, contiguous polynucleotides and comprise an “A” at the nucleotide position corresponding to nucleotide 1350 of the cMOAT gene, or a portion of SEQ ID NO:43. Alternatively, preferred portions are at least 10, preferably at least 20, preferably at least 40, preferably at least 100, contiguous polynucleotides and comprise a “G” at the nucleotide position corresponding to nucleotide 1350 of the cMOAT gene, or a portion of SEQ ID NO:43. The invention further relates to isolated gene products, e.g., polypeptides and/or proteins, which are encoded by a nucleic acid molecule comprising all or a portion of the variant allele of the cMOAT gene.

In one embodiment, the invention relates to a method for predicting the likelihood that an individual will have a disorder associated with an “A” at the nucleotide position corresponding to nucleotide position 1350 of SEQ ID NO:43 (or diagnosing or aiding in the diagnosis of such a disorder) comprising the steps of obtaining a DNA sample from an individual to be assessed and determining the nucleotide present at position 1350 of SEQ ID NO:43. The presence of an “A” at this position indicates that the individual has a greater likelihood of having a disorder associated therewith than an individual having a “G” at that position, or a greater likelihood of having more severe symptoms.

Conversely, the invention relates to a method for predicting the likelihood that an individual will have a disorder associated with a “G” at the nucleotide position corresponding to nucleotide position 1350 of SEQ ID NO:43 (or diagnosing or aiding in the diagnosis of such a disorder) comprising the steps of obtaining a DNA sample from an individual to be assessed and determining the nucleotide present at position 1350 of SEQ ID NO:43. The presence of a “G” at this position indicates that the individual has a greater likelihood of having a disorder associated therewith than an individual having an “A” at that position, or a greater likelihood of having more severe symptoms.

The present invention further relates to isolated proteins or polypeptides comprising, or alternatively, consisting of all or a portion of the encoded variant amino acid sequence of the human human cMOAT, solute carrier family 21 member 6 polypeptide (e.g., wherein reference or wildtype human cMOAT, solute carrier family 21 member 6 polypeptide is exemplified by SEQ ID NO:2). Preferred portions are at least 10, preferably at least 20, preferably at least 40, preferably at least 100, contiguous polypeptides and comprises a “I” at the amino acid position corresponding to amino acid 417 of the cMOAT polypeptide, or a portion of SEQ ID NO:44. Alternatively, preferred portions are at least 10, preferably at least 20, preferably at least 40, preferably at least 100, contiguous polypeptides and comprises a “V” at the amino acid position corresponding to amino acid 417 of the cMOAT protein, or a portion of SEQ ID NO:44. The invention further relates to isolated nucleic acid molecules encoding such polypeptides or proteins, as well as to antibodies that bind to such proteins or polypeptides.

Representative disorders which may be detected, diagnosed, identified, treated, prevented, and/or ameliorated by the present invention include, the following, non-limiting diseases and disorders: decreased heptic uptake of administered statins, resistance to beneficial responses of administered statins; resistance to LDL lowering responses to administered statins; resistance to TG lowering responses to administered statins; resistance to HDL elevating responses to administered statins; increased incidence of drug interactions between one or more administered statins; increased incidence of drug and endogenous substance interactions between a statin drug and endogenous substrates of cMOAT including cholate, taurocholate, thyroid hormones T3 and T4, DHEAS, estradiol-17beta-glucuronide, estrone-3-sulfate, prostaglandin E2, thromboxane B2, leukotriene C4, leukotriene E4, and bilirubin and its conjugates; decreased response to HMG-CoA reductase inhibitors; in addition to other disorders referenced herein, which include, for example, any disorder related to high levels of circulating LDL, high levels of TG, and/or low levels of circularing HDL, which include, but are not limited to, cardiovascular diseases, hepatic diseases, angina pectoris, hypertension, heart failure, myocardial infarction, ventricular hypertrophy, vascular diseases, miscrovascular disease, vascular leak syndrome, aneurysm, stroke, embolism, thrombosis, endothelial dysfunction, coronary artery disease, arteriosclerosis, and/or atherosclerosis.

In preferred embodiments, the following N-terminal cMOAT (SNP_ID:PS101s23) deletion polypeptides are encompassed by the present invention: M1-F1545, L2-F1545, E3-F1545, K4-F1545, F5-F1545, C6-F1545, N7-F1545, S8-F1545, T9-F1545, F10-F1545, W11-F1545, N12-F1545, S13-F1545, S14-F1545, F15-F1545, L16-F1545, D17-F1545, S18-F1545, P19-F1545, E20-F1545, A21-F1545, D22-F1545, L23-F1545, P24-F1545, L25-F1545, C26-F1545, F27-F1545, E28-F1545, Q29-F1545, T30-F1545, V31-F1545, L32-F1545, V33-F1545, W34-F1545, I35-F1545, P36-F1545, L37-F1545, G38-F1545, F39-F1545, L40-F1545, W41-F1545, L42-F1545, L43-F1545, A44-F1545, P45-F1545, W46-F1545, Q47-F1545, L48-F1545, L49-F1545, H50-F1545, V51-F1545, Y52-F1545, K53-F1545, S54-F1545, R55-F1545, T56-F1545, K57-F1545, R58-F1545, S59-F1545, S60-F1545, T61-F1545, T62-F1545, K63-F1545, L64-F1545, Y65-F1545, L66-F1545, A67-F1545, K68-F1545, Q69-F1545, V70-F1545, F71-F1545, V72-F1545, G73-F1545, F74-F1545, L75-F1545, L76-F1545, I77-F1545, L78-F1545, A79-F1545, A80-F1545, I81-F1545, E82-F1545, L83-F1545, A84-F1545, L85-F1545, V86-F1545, L87-F1545, T88-F1545, E89-F1545, D90-F1545, S91-F1545, G92-F1545, Q93-F1545, A94-F1545, T95-F1545, V96-F1545, P97-F1545, A98-F1545, V99-F1545, R100-F1545, Y101-F1545, T102-F1545, N103-F1545, P104-F1545, S105-F1545, L106-F1545, Y107-F1545, L108-F1545, G109-F1545, T110-F1545, W111-F1545, L112-F1545, L113-F1545, V114-F1545, L115-F1545, L116-F1545, I117-F1545, Q118-F1545, Y119-F1545, S120-F1545, R121-F1545, Q122-F1545, W123-F1545, C124-F1545, V125-F1545, Q126-F1545, K127-F1545, N128-F1545, S129-F1545, W130-F1545, F131-F1545, L132-F1545, S133-F1545, L134-F1545, F135-F1545, W136-F1545, I137-F1545, L138-F1545, S139-F1545, I140-F1545, L141-F1545, C142-F1545, G143-F1545, T144-F1545, F145-F1545, Q146-F1545, F147-F1545, Q148-F1545, T149-F1545, L150-F1545, I151-F1545, R152-F1545, T153-F1545, L154-F1545, L155-F1545, Q156-F1545, G157-F1545, D158-F1545, N159-F1545, S160-F1545, N161-F1545, L162-F1545, A163-F1545, Y164-F1545, S165-F1545, C166-F1545, L167-F1545, F168-F1545, F169-F1545, I170-F1545, S171-F1545, Y172-F1545, G173-F1545, F174-F1545, Q175-F1545, I176-F1545, L177-F1545, I178-F1545, L179-F1545, I180-F1545, F181-F1545, S182-F1545, A183-F1545, F184-F1545, S185-F1545, E186-F1545, N187-F1545, N188-F1545, E189-F1545, S190-F1545, S191-F1545, N192-F1545, N193-F1545, P194-F1545, S195-F1545, S196-F1545, I197-F1545, A198-F1545, S199-F1545, F200-F1545, L201-F1545, S202-F1545, S203-F1545, I204-F1545, T205-F1545, Y206-F1545, S207-F1545, W208-F1545, Y209-F1545, D210-F1545, S211-F1545, I212-F1545, I213-F1545, L214-F1545, K215-F1545, G216-F1545, Y217-F1545, K218-F1545, R219-F1545, P220-F1545, L221-F1545, T222-F1545, L223-F1545, E224-F1545, D225-F1545, V226-F1545, W227-F1545, E228-F1545, V229-F1545, D230-F1545, E231-F1545, E232-F1545, M233-F1545, K234-F1545, T235-F1545, K236-F1545, T237-F1545, L238-F1545, V239-F1545, S240-F1545, K241-F1545, F242-F1545, E243-F1545, T244-F1545, H245-F1545, M246-F1545, K247-F1545, R248-F1545, E249-F1545, L250-F1545, Q251-F1545, K252-F1545, A253-F1545, R254-F1545, R255-F1545, A256-F1545, L257-F1545, Q258-F1545, R259-F1545, R260-F1545, Q261-F1545, E262-F1545, K263-F1545, S264-F1545, S265-F1545, Q266-F1545, Q267-F1545, N268-F1545, S269-F1545, G270-F1545, A271-F1545, R272-F1545, L273-F1545, P274-F1545, G275-F1545, L276-F1545, N277-F1545, K278-F1545, N279-F1545, Q280-F1545, S281-F1545, Q282-F1545, S283-F1545, Q284-F1545, D285-F1545, A286-F1545, L287-F1545, V288-F1545, L289-F1545, E290-F1545, D291-F1545, V292-F1545, E293-F1545, K294-F1545, K295-F1545, K296-F1545, K297-F1545, K298-F1545, S299-F1545, G300-F1545, T301-F1545, K302-F1545, K303-F1545, D304-F1545, V305-F1545, P306-F1545, K307-F1545, S308-F1545, W309-F1545, L310-F1545, M311-F1545, K312-F1545, A313-F1545, L314-F1545, F315-F1545, K316-F1545, T317-F1545, F318-F1545, Y319-F1545, M320-F1545, V321-F1545, L322-F1545, L323-F1545, K324-F1545, S325-F1545, F326-F1545, L327-F1545, L328-F1545, K329-F1545, L330-F1545, V331-F1545, N332-F1545, D333-F1545, I334-F1545, F335-F1545, T336-F1545, F337-F1545, V338-F1545, S339-F1545, P340-F1545, Q341-F1545, L342-F1545, L343-F1545, K344-F1545, L345-F1545, L346-F1545, I347-F1545, S348-F1545, F349-F1545, A350-F1545, S351-F1545, D352-F1545, R353-F1545, D354-F1545, T355-F1545, Y356-F1545, L357-F1545, W358-F1545, I359-F1545, G360-F1545, Y361-F1545, L362-F1545, C363-F1545, A364-F1545, I365-F1545, L366-F1545, L367-F1545, F368-F1545, T369-F1545, A370-F1545, A371-F1545, L372-F1545, I373-F1545, Q374-F1545, S375-F1545, F376-F1545, C377-F1545, L378-F1545, Q379-F1545, C380-F1545, Y381-F1545, F382-F1545, Q383-F1545, L384-F1545, C385-F1545, F386-F1545, K387-F1545, L388-F1545, G389-F1545, V390-F1545, K391-F1545, V392-F1545, R393-F1545, T394-F1545, A395-F1545, I396-F1545, M397-F1545, A398-F1545, S399-F1545, V400-F1545, Y401-F1545, K402-F1545, K403-F1545, A404-F1545, L405-F1545, T406-F1545, L407-F1545, S408-F1545, N409-F1545, L410-F1545, A411-F1545, R412-F1545, K413-F1545, E414-F1545, Y415-F1545, T416-F1545, and/or 1417-F1545 of SEQ ID NO:44. Polynucleotide sequences encoding these polypeptides are also provided. The present invention also encompasses the use of these N-terminal cMOAT (SNP_ID:PS101s23) deletion polypeptides as immunogenic and/or antigenic epitopes as described elsewhere herein.

In preferred embodiments, the following C-terminal cMOAT (SNP_ID:PS101s23) deletion polypeptides are encompassed by the present invention: M1-F1545, M1-K1544, M1-T1543, M1-S1542, M1-N1541, M1-V1540, M1-N1539, M1-E1538, M1-I1537, M1-G1536, M1-A1535, M1-E1534, M1-K1533, M1-A1532, M1-M1531, M1-F1530, M1-Y1529, M1-F1528, M1-P1527, M1-G1526, M1-P1525, M1-I1524, M1-Q1523, M1-L1522, M1-L1521, M1-E1520, M1-E1519, M1-P1518, M1-S1517, M1-G1516, M1-Y1515, M1-E1514, M1-I1513, M1-I1512, M1-K1511, M1-G1510, M1-N1509, M1-D1508, M1-L1507, M1-V1506, M1-M1505, M1-V1504, M1-K1503, M1-D1502, M1-S1501, M1-D1500, M1-M1499, M1-I1498, M1-T1497, M1-H1496, M1-L1495, M1-R1494, M1-H1493, M1-A1492, M1-I1491, M1-T1490, M1-I1489, M1-V1488, M1-T1487, M1-C1486, M1-H1485, M1-A1484, M1-F1483, M1-E1482, M1-N1481, M1-Q1480, M1-I1479, M1-T1478, M1-T1477, M1-Q1476, M1-I1475, M1-L1474, M1-N1473, M1-D1472, M1-T1471, M1-E1470, M1-L1469, M1-D1468, M1-V1467, M1-A1466, M1-A1465, M1-T1464, M1-A1463, M1-E1462, M1-D1461, M1-L1460, M1-V1459, M1-L1458, M1-I1457, M1-K1456, M1-S1455, M1-K1454, M1-R1453, M1-L1452, M1-L1451, M1-A1450, M1-R1449, M1-G1448, M1-L1447, M1-C1446, M1-L1445, M1-L1444, M1-Q1443, M1-R1442, M1-Q1441, M1-G1440, M1-I1439, M1-S1438, M1-L1437, M1-N1436, M1-G1435, M1-G1434, M1-A1433, M1-E1432, M1-T1431, M1-V1430, M1-E1429, M1-H1428, M1-S1427, M1-L1426, M1-G1425, M1-L1424, M1-Q1423, M1-L1422, M1-S1421, M1-A1420, M1-V1419, M1-F1418, M1-S1417, M1-K1416, M1-L1415, M1-H1414, M1-A1413, M1-L1412, M1-E1411, M1-L1410, M1-A1409, M1-K1408, M1-W1407, M1-I1406, M1-E1405, M1-E1404, M1-D1403, M1-S1402, M1-Y1401, M1-N1400, M1-N1399, M1-F1398, M1-P1397, M1-D1396, M1-L1395, M1-N1394, M1-M1393, M1-R1392, M1-L1391, M1-S1390, M1-G1389, M1-S1388, M1-F1387, M1-L1386, M1-I1385, M1-P1384, M1-D1383, M1-Q1382, M1-P1381, M1-I1380, M1-I1379, M1-T1378, M1-L1377, M1-K1376, M1-E1375, M1-R1374, M1-L1373, M1-D1372, M1-H1371, M1-L1370, M1-G1369, M1-I1368, M1-S1367, M1-A1366, M1-I1365, M1-D1364, M1-V1363, M1-G1362, M1-D1361, M1-I1360, M1-I1359, M1-I1358, M1-Q1357, M1-G1356, M1-G1355, M1-A1354, M1-A1353, M1-E1352, M1-L1351, M1-I1350, M1-R1349, M1-F1348, M1-L1347, M1-C1346, M1-N1345, M1-T1344, M1-L1343, M1-S1342, M1-S1341, M1-K1340, M1-G1339, M1-A1338, M1-G1337, M1-T1336, M1-R1335, M1-G1334, M1-V1333, M1-V1332, M1-G1331, M1-I1330, M1-K1329, M1-E1328, M1-M1327, M1-S1326, M1-G1325, M1-I1324, M1-D1323, M1-C1322, M1-T1321, M1-I1320, M1-G1319, M1-R1318, M1-L1317, M1-V1316, M1-L1315, M1-D1314, M1-L1313, M1-E1312, M1-P1311, M1-R1310, M1-Y1309, M1-R1308, M1-V1307, M1-Q1306, M1-Y1305, M1-N1304, M1-N1303, M1-F1302, M1-Q1301, M1-I1300, M1-K1299, M1-G1298, M1-K1297, M1-S1296, M1-P1295, M1-W1294, M1-D1293, M1-P1292, M1-P1291, M1-P1290, M1-R1289, M1-K1288, M1-D1287, M1-T1286, M1-V1285, M1-W1284, M1-P1283, M1-A1282, M1-E1281, M1-N1280, M1-E1279, M1-V1278, M1-K1277, M1-T1276, M1-Y1275, M1-E1274, M1-T1273, M1-I1272, M1-R1271, M1-E1270, M1-V1269, M1-A1268, M1-V1267, M1-I1266, M1-N1265, M1-T1264, M1-E1263, M1-I1262, M1-E1261, M1-S1260, M1-T1259, M1-M1258, M1-R1257, M1-V1256, M1-L1255, M1-W1254, M1-N1253, M1-L1252, M1-T1251, M1-Q1250, M1-T1249, M1-I1248, M1-N1247, M1-L1246, M1-A1245, M1-N1244, M1-S1243, M1-L1242, M1-V1241, M1-F1240, M1-G1239, M1-V1238, M1-T1237, M1-D1236, M1-G1235, M1-S1234, M1-L1233, M1-T1232, M1-D1231, M1-R1230, M1-Y1229, M1-I1228, M1-V1227, M1-M1226, M1-M1225, M1-L1224, M1-A1223, M1-S1222, M1-F1221, M1-F1220, M1-V1219, M1-T1218, M1-L1217, M1-N1216, M1-G1215, M1-V1214, M1-L1213, M1-E1212, M1-L1211, M1-R1210, M1-I1209, M1-A1208, M1-L1207, M1-W1206, M1-R1205, M1-N1204, M1-S1203, M1-T1202, M1-I1201, M1-W1200, M1-S1199, M1-F1198, M1-V1197, M1-C1196, M1-K1195, M1-Q1194, M1-N1193, M1-T1192, M1-D1191, M1-I1190, M1-R1189, M1-E1188, M1-E1187, M1-N1186, M1-H1185, M1-K1184, M1-L1183, M1-F1182, M1-R1181, M1-Q1180, M1-Q1179, M1-H1178, M1-E1177, M1-F1176, M1-A1175, M1-R1174, M1-I1173, M1-V1172, M1-P1171, M1-L1170, M1-G1169, M1-S1168, M1-V1167, M1-T1166, M1-E1165, M1-S1164, M1-F1163, M1-H1162, M1-S1161, M1-Y1160, M1-I1159, M1-P1158, M1-S1157, M1-R1156, M1-T1155, M1-V1154, M1-S1153, M1-D1152, M1-L1151, M1-R1150, M1-R1149, M1-L1148, M1-Q1147, M1-R1146, M1-S1145, M1-T1144, M1-S1143, M1-V1142, M1-Y1141, M1-F1140, M1-M1139, M1-Q1138, M1-V1137, M1-S1136, M1-V1135, M1-Y1134, M1-I1133, M1-I1132, M1-G1131, M1-L1130, M1-P1129, M1-I1128, M1-V1127, M1-I1126, M1-I1125, M1-T1124, M1-F1123, M1-V1122, M1-P1121, M1-T1120, M1-A1119, M1-M1118, M1-C1117, M1-I1116, M1-M1115, M1-V1114, M1-L1113, M1-T1112, M1-S1111, M1-I1110, M1-I1109, M1-G1108, M1-L1107, M1-F1106, M1-C1105, M1-T1104, M1-I1103, M1-W1102, M1-S1101, M1-R1100, M1-L1099, M1-S1098, M1-Q1097, M1-P1096, M1-L1095, M1-T1094, M1-D1093, M1-D1092, M1-V1091, M1-T1090, M1-S1089, M1-I1088, M1-D1087, M1-G1086, M1-A1085, M1-F1084, M1-R1083, M1-N1082, M1-V1081, M1-I1080, M1-R1079, M1-G1078, M1-T1077, M1-P1076, M1-T1075, M1-T1074, M1-D1073, M1-F1072, M1-F1071, M1-R1070, M1-M1069, M1-P1068, M1-A1067, M1-R1066, M1-L1065, M1-I1064, M1-N1063, M1-N1062, M1-L1061, M1-L1060, M1-Q1059, M1-K1058, M1-H1057, M1-L1056, M1-I1055, M1-N1054, M1-S1053, M1-A1052, M1-H1051, M1-V1050, M1-F1049, M1-G1048, M1-F1047, M1-A1046, M1-S1045, M1-W1044, M1-F1043, M1-H1042, M1-A1041, M1-I1040, M1-F1039, M1-V1038, M1-F1037, M1-I1036, M1-G1035, M1-Q1034, M1-A1033, M1-L1032, M1-G1031, M1-L1030, M1-A1029, M1-G1028, M1-Y1027, M1-V1026, M1-G1025, M1-V1024, M1-R1023, M1-M1022, M1-D1021, M1-R1020, M1-Q1019, M1-S1018, M1-A1017, M1-P1016, M1-Y1015, M1-D1014, M1-T1013, M1-S1012, M1-N1011, M1-F1010, M1-I1009, M1-K1008, M1-S1007, M1-D1006, M1-S1005, M1-T1004, M1-W1003, M1-A1002, M1-S1001, M1-L1000, M1-W999, M1-L998, M1-N997, M1-S996, M1-G995, M1-I994, M1-F993, M1-A992, M1-V991, M1-S990, M1-N989, M1-M988, M1-V987, M1-F986, M1-A985, M1-L984, M1-I983, M1-I982, M1-F981, M1-F980, M1-I979, M1-S978, M1-F977, M1-L976, M1-G975, M1-I974, M1-A973, M1-Q972, M1-L971, M1-Y970, M1-E969, M1-L968, M1-Y967, M1-I966, M1-S965, M1-F964, M1-K963, M1-V962, M1-K961, M1-G960, M1-T959, M1-E958, M1-I957, M1-F956, M1-E955, M1-K954, M1-K953, M1-I952, M1-L951, M1-K950, M1-Q949, M1-G948, M1-K947, M1-V946, M1-L945, M1-E944, M1-E943, M1-D942, M1-E941, M1-K940, M1-L939, M1-S938, M1-N937, M1-V936, M1-N935, M1-R934, M1-T933, M1-K932, M1-L931, M1-S930, M1-N929, M1-R928, M1-L927, M1-S926, M1-K925, M1-L924, M1-H923, M1-R922, M1-G921, M1-N920, M1-S919, M1-R918, M1-S917, M1-S916, M1-R915, M1-S914, M1-L913, M1-T912, M1-R911, M1-R910, M1-F909, M1-S908, M1-N907, M1-E906, M1-R905, M1-R904, M1-M903, M1-T902, M1-I901, M1-S900, M1-A899, M1-A898, M1-D897, M1-E896, M1-P895, M1-I894, M1-E893, M1-E892, M1-V891, M1-S890, M1-S889, M1-I888, M1-L887, M1-G886, M1-Y885, M1-D884, M1-D883, M1-D882, M1-E881, M1-E880, M1-E879, M1-S878, M1-G877, M1-D876, M1-H875, M1-V874, M1-T873, M1-A872, M1-E871, M1-E870, M1-E869, M1-P868, M1-G867, M1-T866, M1-H865, M1-R864, M1-L863, M1-F862, M1-T861, M1-K860, M1-L859, M1-N858, M1-K857, M1-A856, M1-F855, M1-E854, M1-G853, M1-K852, M1-K851, M1-A850, M1-L849, M1-L848, M1-A847, M1-S846, M1-Y845, M1-S844, M1-G843, M1-K842, M1-E841, M1-V840, M1-I839, M1-T838, M1-G837, M1-N836, M1-G835, M1-L834, M1-V833, M1-V832, M1-I831, M1-E830, M1-D829, M1-V828, M1-Q827, M1-P826, M1-L825, M1-F824, M1-H823, M1-M822, M1-S821, M1-H820, M1-T819, M1-V818, M1-L817, M1-L816, M1-R815, M1-T814, M1-K813, M1-G812, M1-K811, M1-L810, M1-L809, M1-G808, M1-N807, M1-P806, M1-G805, M1-L804, M1-V803, M1-K802, M1-N801, M1-F800, M1-I799, M1-H798, M1-K797, M1-G796, M1-V795, M1-H794, M1-A793, M1-D792, M1-V791, M1-A790, M1-S789, M1-L788, M1-P787, M1-D786, M1-D785, M1-L784, M1-L783, M1-Y782, M1-I781, M1-D780, M1-L779, M1-N778, M1-Q777, M1-Y776, M1-T775, M1-A774, M1-R773, M1-A772, M1-L771, M1-S770, M1-I769, M1-R768, M1-Q767, M1-K766, M1-Q765, M1-G764, M1-G763, M1-S762, M1-L761, M1-N760, M1-I759, M1-G758, M1-K757, M1-E756, M1-G755, M1-I754, M1-E753, M1-A752, M1-L751, M1-D750, M1-G749, M1-G748, M1-P747, M1-L746, M1-M745, M1-E744, M1-L743, M1-D742, M1-P741, M1-L740, M1-L739, M1-A738, M1-C737, M1-A736, M1-E735, M1-L734, M1-V733, M1-Q732, M1-Q731, M1-Y730, M1-R729, M1-K728, M1-E727, M1-N726, M1-F725, M1-E724, M1-T723, M1-G722, M1-F721, M1-L720, M1-I719, M1-N718, M1-D717, M1-K716, M1-I715, M1-T714, M1-G713, M1-N712, M1-Q711, M1-I710, M1-W709, M1-S708, M1-Q707, M1-Q706, M1-P705, M1-V704, M1-Y703, M1-A702, M1-T701, M1-T700, M1-G699, M1-K698, M1-I697, M1-T696, M1-I695, M1-H694, M1-G693, M1-H692, M1-V691, M1-N690, M1-E689, M1-M688, M1-E687, M1-G686, M1-L685, M1-M684, M1-A683, M1-S682, M1-I681, M1-L680, M1-S679, M1-S678, M1-K677, M1-G676, M1-S675, M1-G674, M1-V673, M1-P672, M1-G671, M1-I670, M1-V669, M1-A668, M1-V667, M1-L666, M1-Q665, M1-G664, M1-A663, M1-M662, M1-I661, M1-D660, M1-L659, M1-N658, M1-V657, M1-D656, M1-R655, M1-V654, M1-T653, M1-A652, M1-E651, M1-S650, M1-D649, M1-H648, M1-E647, M1-W646, M1-T645, M1-F644, M1-S643, M1-A642, M1-E641, M1-S640, M1-F639, M1-Q638, M1-M637, M1-A636, M1-K635, M1-D634, M1-F633, M1-N632, M1-C631, M1-D630, M1-H629, M1-R628, M1-I627, M1-A626, M1-S625, M1-T624, M1-D623, M1-L622, M1-D621, M1-D620, M1-G619, M1-G618, M1-L617, M1-Y616, M1-K615, M1-E614, M1-L613, M1-R612, M1-E611, M1-T610, M1-S609, M1-V608, M1-S607, M1-A606, M1-Q605, M1-L604, M1-M603, M1-S602, M1-S601, M1-I600, M1-M599, M1-M598, M1-P597, M1-L596, M1-M595, M1-S594, M1-L593, M1-P592, M1-F591, M1-R590, M1-L589, M1-I588, M1-N587, M1-F586, M1-L585, M1-T584, M1-I583, M1-S582, M1-T581, M1-F580, M1-A579, M1-K578, M1-Q577, M1-A576, M1-D575, M1-L574, M1-I573, M1-N572, M1-N571, M1-S570, M1-D569, M1-V568, M1-L567, M1-V566, M1-Y565, M1-V564, M1-S563, M1-F562, M1-T561, M1-V560, M1-V559, M1-S558, M1-V557, M1-L556, M1-V555, M1-P554, M1-T553, M1-L552, M1-Q551, M1-F550, M1-V549, M1-F548, M1-I547, M1-V546, M1-V545, M1-C544, M1-Q551, M1-L542, M1-Q541, M1-S540, M1-F539, M1-A538, M1-L537, M1-L536, M1-N535, M1-K534, M1-L533, M1-E532, M1-K531, M1-K530, M1-R529, M1-L528, M1-N527, M1-Q526, M1-V525, M1-Q524, M1-D523, M1-R522, M1-F521, M1-S520, M1-P519, M1-E518, M1-W517, M1-A516, M1-F515, M1-Y514, M1-K513, M1-L512, M1-I511, M1-K510, M1-I509, M1-G508, M1-S507, M1-L506, M1-I505, M1-E504, M1-N503, M1-M502, M1-I501, M1-K500, M1-L499, M1-R498, M1-K497, M1-D496, M1-K495, M1-N494, M1-K493, M1-M492, M1-N491, M1-K490, M1-V489, M1-Q488, M1-I487, M1-T486, M1-K485, M1-S484, M1-K483, M1-T482, M1-S481, M1-L480, M1-I479, M1-A478, M1-N477, M1-I476, M1-P475, M1-I474, M1-V473, M1-L472, M1-V471, M1-M470, M1-V469, M1-G468, M1-V467, M1-G466, M1-A465, M1-L464, M1-V463, M1-S462, M1-P461, M1-G460, M1-L459, M1-E458, M1-R457, M1-W456, M1-L455, M1-F454, M1-F453, M1-I452, M1-S451, M1-L450, M1-V449, M1-I448, M1-Q447, M1-L446, M1-V445, M1-S444, M1-S443, M1-W442, M1-L441, M1-M440, M1-H439, M1-M438, M1-F437, M1-N436, M1-T435, M1-V434, M1-D433, M1-M432, M1-L431, M1-K430, M1-Q429, M1-A428, M1-D427, M1-V426, M1-S425, M1-M424, M1-L423, M1-N422, M1-V421, M1-T420, M1-E419, M1-G418, and/or M1-I417 of SEQ ID NO:44. Polynucleotide sequences encoding these polypeptides are also provided. The present invention also encompasses the use of these C-terminal cMOAT (SNP_ID:PS101s23) deletion polypeptides as immunogenic and/or antigenic epitopes as described elsewhere herein.

Alternatively, preferred polypeptides of the present invention may comprise polypeptide sequences corresponding to, for example, internal regions of the cMOAT (SNP_ID: PS101s23) polypeptide (e.g., any combination of both N- and C-terminal cMOAT (SNP_ID: PS101s23) polypeptide deletions) of SEQ ID NO:44. For example, internal regions could be defined by the equation: amino acid NX to amino acid CX, wherein NX refers to any N-terminal deletion polypeptide amino acid of cMOAT (SNP_ID: PS101s23) (SEQ ID NO:44), and where CX refers to any C-terminal deletion polypeptide amino acid of cMOAT (SNP_ID: PS101s23) (SEQ ID NO:44). Polynucleotides encoding these polypeptides are also provided. The present invention also encompasses the use of these polypeptides as an immunogenic and/or antigenic epitope as described elsewhere herein. Preferably, the resulting deletion polypeptide comprises the polypeptide polymorphic loci identified elsewhere herein for cMOAT (SNP_ID: PS101s23), and more preferably comprises the polypeptide polymorphic allele identified elsewhere herein for cMOAT (SNP_ID: PS 101s23).

### Features of the Polypeptide Encoded by Gene No:21

The present invention relates to isolated nucleic acid molecules comprising, or alternatively, consisting of all or a portion of the variant allele of the human cMOAT, ATP-binding cassette sub-family C member 2 gene (SNP_ID: PS101s24) (e.g., wherein reference or wildtype cMOAT gene is exemplified by SEQ ID NO:1). Preferred portions are at least 10, preferably at least 20, preferably at least 40, preferably at least 100, contiguous polynucleotides and comprise a “T at the nucleotide position corresponding to nucleotide 1320 of the cMOAT gene, or a portion of SEQ ID NO:45. Alternatively, preferred portions are at least 10, preferably at least 20, preferably at least 40, preferably at least 100, contiguous polynucleotides and comprise a “C” at the nucleotide position corresponding to nucleotide 1320 of the cMOAT gene, or a portion of SEQ ID NO:45. The invention further relates to isolated gene products, e.g., polypeptides and/or proteins, which are encoded by a nucleic acid molecule comprising all or a portion of the variant allele of the cMOAT gene.

In one embodiment, the invention relates to a method for predicting the likelihood that an individual will have a disorder associated with a “T” at the nucleotide position corresponding to nucleotide position 1320 of SEQ ID NO:45 (or diagnosing or aiding in the diagnosis of such a disorder) comprising the steps of obtaining a DNA sample from an individual to be assessed and determining the nucleotide present at position 1320 of SEQ ID NO:45. The presence of a “T” at this position indicates that the individual has a greater likelihood of having a disorder associated therewith than an individual having a “C” at that position, or a greater likelihood of having more severe symptoms.

Conversely, the invention relates to a method for predicting the likelihood that an individual will have a disorder associated with a “C” at the nucleotide position corresponding to nucleotide position 1320 of SEQ ID NO:45 (or diagnosing or aiding in the diagnosis of such a disorder) comprising the steps of obtaining a DNA sample from an individual to be assessed and determining the nucleotide present at position 1320 of SEQ ID NO:45. The presence of a “C” at this position indicates that the individual has a greater likelihood of having a disorder associated therewith than an individual having a “T” at that position, or a greater likelihood of having more severe symptoms.

The present invention further relates to isolated proteins or polypeptides comprising, or alternatively, consisting of all or a portion of the encoded variant amino acid sequence of the human human cMOAT, solute carrier family 21 member 6 polypeptide (e.g., wherein reference or wildtype human cMOAT, solute carrier family 21 member 6 polypeptide is exemplified by SEQ ID NO:2). Preferred portions are at least 10, preferably at least 20, preferably at least 40, preferably at least 100, contiguous polypeptides and comprises a “K” at the amino acid position corresponding to amino acid 407 of the cMOAT polypeptide, or a portion of SEQ ID NO:46. Alternatively, preferred portions are at least 10, preferably at least 20, preferably at least 40, preferably at least 100, contiguous polypeptides and comprises a “L” at the amino acid position corresponding to amino acid 407 of the cMOAT protein, or a portion of SEQ ID NO:46. The invention further relates to isolated nucleic acid molecules encoding such polypeptides or proteins, as well as to antibodies that bind to such proteins or polypeptides.

Representative disorders which may be detected, diagnosed, identified, treated, prevented, and/or ameliorated by the present invention include, the following, non-limiting diseases and disorders: decreased heptic uptake of administered statins, resistance to beneficial responses of administered statins; resistance to LDL lowering responses to administered statins; resistance to TG lowering responses to administered statins; resistance to HDL elevating responses to administered statins; increased incidence of drug interactions between one or more administered statins; increased incidence of drug and endogenous substance interactions between a statin drug and endogenous substrates of cMOAT including cholate, taurocholate, thyroid hormones T3 and T4, DHEAS, estradiol-17beta-glucuronide, estrone-3-sulfate, prostaglandin E2, thromboxane B2, leukotriene C4, leukotriene E4, and bilirubin and its conjugates; decreased response to HMG-CoA reductase inhibitors; in addition to other disorders referenced herein, which include, for example, any disorder related to high levels of circulating LDL, high levels of TG, and/or low levels of circularing HDL, which include, but are not limited to, cardiovascular diseases, hepatic diseases, angina pectoris, hypertension, heart failure, myocardial infarction, ventricular hypertrophy, vascular diseases, miscrovascular disease, vascular leak syndrome, aneurysm, stroke, embolism, thrombosis, endothelial dysfunction, coronary artery disease, arteriosclerosis, and/or atherosclerosis.

In preferred embodiments, the following N-terminal cMOAT (SNP_ID:PS101s24) deletion polypeptides are encompassed by the present invention: M1-F1545, L2-F1545, E3-F1545, K4-F1545, F5-F1545, C6-F1545, N7-F1545, S8-F1545, T9-F1545, F10-F1545, W11-F1545, N12-F1545, S13-F1545, S14-F1545, F15-F1545, L16-F1545, D17-F1545, S18-F1545, P19-F1545, E20-F1545, A21-F1545, D22-F1545, L23-F1545, P24-F1545, L25-F1545, C26-F1545, F27-F1545, E28-F1545, Q29-F1545, T30-F1545, V31-F1545, L32-F1545, V33-F1545, W34-F1545, I35-F1545, P36-F1545, L37-F1545, G38-F1545, F39-F1545, L40-F1545, W41-F1545, L42-F1545, L43-F1545, A44-F1545, P45-F1545, W46-F1545, Q47-F1545, L48-F1545, L49-F1545, H50-F1545, V51-F1545, Y52-F1545, K53-F1545, S54-F1545, R55-F1545, T56-F1545, K57-F1545, R58-F1545, S59-F1545, S60-F1545, T61-F1545, T62-F1545, K63-F1545, L64-F1545, Y65-F1545, L66-F1545, A67-F1545, K68-F1545, Q69-F1545, V70-F1545, F71-F1545, V72-F1545, G73-F1545, F74-F1545, L75-F1545, L76-F1545, I77-F1545, L78-F1545, A79-F1545, A80-F1545, I81-F.545, E82-F1545, L83-F1545, A84-F1545, L85-F1545, V86-F1545, L87-F1545, T88-F1545, E89-F1545, D90-F1545, S91-F1545, G92-F1545, Q93-F1545, A94-F1545, T95-F1545, V96-F1545, P97-F1545, A98-F1545, V99-F1545, R100-F1545, Y10-F1545, T102-F1545, N103-F1545, P104-F1545, S105-F1545, L106-F1545, Y107-F1545, L108-F1545, G109-F1545, T110-F1545, W111-F1545, L112-F1545, L113-F1545, V114-F1545, L115-F1545, L116-F1545, I117-F1545, Q118-F1545, Y119-F1545, S120-F1545, R121-F1545, Q122-F1545, W123-F1545, C124-F1545, V125-F1545, Q126-F1545, K127-F1545, N128-F1545, S129-F1545, W130-F1545, F131-F1545, L132-F1545, S133-F1545, L134-F1545, F135-F1545, W136-F1545, I137-F1545, L138-F1545, S139-F1545, I140-F1545, L141-F1545, C142-F1545, G143-F1545, T144-F1545, F145-F1545, Q146-F1545, F147-F1545, Q148-F1545, T149-F1545, L150-F1545, I151-F1545, R152-F1545, T153-F1545, L154-F1545, L155-F1545, Q156-F1545, G157-F1545, D158-F1545, N159-F1545, S160-F1545, N161-F1545, L162-F1545, A163-F1545, Y164-F1545, S165-F1545, C166-F1545, L167-F1545, F168-F1545, F169-F1545, I170-F1545, S171-F1545, Y172-F1545, G173-F1545, F174-F1545, Q175-F1545, I176-F1545, L177-F1545, I178-F1545, L179-F1545, I180-F1545, F181-F1545, S182-F1545, A183-F1545, F184-F1545, S185-F1545, E186-F1545, N187-F1545, N188-F1545, E189-F1545, S196-F1545, I197-F1545, A198-F1545, S199-F1545, F200-F1545, L201-F1545, S202-F1545, S203-F1545, I204-F1545, T205-F1545, Y206-F1545, S207-F1545, W208-F1545, Y209-F1545, D210-F1545, S211-F1545, I212-F1545, I213-F1545, L214-F1545, K215-F1545, G216-F1545, Y217-F1545, K218-F1545, R219-F1545, P220-F1545, L221-F1545, T222-F1545, L223-F1545, E224-F1545, D225-F1545, V226-F1545, W227-F1545, E228-F1545, V229-F1545, D230-F1545, E231-F1545, E232-F1545, M233-F1545, K234-F1545, T235-F1545, K236-F1545, T237-F1545, L238-F1545, V239-F1545, S240-F1545, K241-F1545, F242-F1545, E243-F1545, T244-F1545, H245-F1545, M246-F1545, K247-F1545, R248-F1545, E249-F1545, L250-F1545, Q251-F1545, K252-F1545, A253-F1545, R254-F1545, R255-F1545, A256-F1545, L257-F1545, Q258-F1545, R259-F1545, R260-F1545, Q261-F1545, E262-F1545, K263-F1545, S264-F1545, S265-F1545, Q266-F1545, Q267-F1545, N268-F1545, S269-F1545, G270-F1545, A271-F1545, R272-F1545, L273-F1545, P274-F1545, G275-F1545, L276-F1545, N277-F1545, K278-F1545, N279-F1545, Q280-F1545, S281-F1545, Q282-F1545, S283-F1545, Q284-F1545, D285-F1545, A286-F1545, L287-F1545, V288-F1545, L289-F1545, E290-F1545, D291-F1545, V292-F1545, E293-F1545, K294-F1545, K295-F1545, K296-F1545, K297-F1545, K298-F1545, S299-F1545, G300-F1545, T301-F1545, K302-F1545, K303-F1545, D304-F1545, V305-F1545, P306-F1545, K307-F1545, S308-F1545, W309-F1545, L310-F1545, M311-F1545, K312-F1545, A313-F1545, L314-F1545, F315-F1545, K316-F1545, T317-F1545, F318-F1545, Y319-F1545, M320-F1545, V321-F1545, L322-F1545, L323-F1545, K324-F1545, S325-F1545, F326-F1545, L327-F1545, L328-F1545, K329-F1545, L330-F1545, V331-F1545, N332-F1545, D333-F1545, I334-F1545, F335-F1545, T336-F1545, F337-F1545, V338-F1545, S339-F1545, P340-F1545, Q341-F1545, L342-F1545, L343-F1545, K344-F1545, L345-F1545, L346-F1545, I347-F1545, S348-F1545, F349-F1545, A350-F1545, S351-F1545, D352-F1545, R353-F1545, D354-F1545, T355-F1545, Y356-F1545, L357-F1545, W358-F1545, I359-F1545, G360-F1545, Y361-F1545, L362-F1545, C363-F1545, A370-F1545, A371-F1545, L372-F1545, I373-F1545, Q374-F1545, S375-F1545, F376-F1545, C377-F1545, L378-F1545, Q379-F1545, C380-F1545, Y381-F1545, F382-F1545, Q383-F1545, L384-F1545, C385-F1545, F386-F1545, K387-F1545, L388-F1545, G389-F1545, V390-F1545, K391-F1545, V392-F1545, R393-F1545, T394-F1545, A395-F1545, I396-F1545, M397-F1545, A398-F1545, S399-F1545, V400-F1545, Y401-F1545, K402-F1545, K403-F1545, A404-F1545, L405-F1545, T406-F1545, and/or K407-F1545 of SEQ ID NO:46. Polynucleotide sequences encoding these polypeptides are also provided. The present invention also encompasses the use of these N-terminal cMOAT (SNP_ID:PS101s24) deletion polypeptides as immunogenic and/or antigenic epitopes as described elsewhere herein.

In preferred embodiments, the following C-terminal cMOAT (SNP_ID:PS101s24) deletion polypeptides are encompassed by the present invention: M1-F1545, M1-K1544, M1-T1543, M1-S1542, M1-N1541, M1-V1540, M1-N1539, M1-E1538, M1-I1537, M1-G1536, M1-A1535, M1-E1534, M1-K1533, M1-A1532, M1-M1531, M1-F1530, M1-Y1529, M1-F1528, M1-P1527, M1-G1526, M1-P1525, M1-I1524, M1-Q1523, M1-L1522, M1-L1521, M1-E1520, M1-E1519, M1-P1518, M1-S1517, M1-G1516, M1-Y1515, M1-E1514, M1-I1513, M1-I1512, M1-K1511, M1-G1510, M1-N1509, M1-D1508, M1-L1507, M1-V1506, M1-M1505, M1-V1504, M1-K1503, M1-D1502, M1-S1501, M1-D1500, M1-M1499, M1-I1498, M1-T1497, M1-H1496, M1-L1495, M1-R1494, M1-H1493, M1-A1492, M1-I1491, M1-T1490, M1-I1489, M1-V1488, M1-T1487, M1-C1486, M1-H1485, M1-A1484, M1-F1483, M1-E1482, M1-N1481, M1-Q1480, M1-I1479, M1-T1478, M1-T1477, M1-Q1476, M1-I1475, M1-L1474, M1-N1473, M1-D1472, M1-T1471, M1-E1470, M1-L1469, M1-D1468, M1-V1467, M1-A1466, M1-A1465, M1-T1464, M1-A1463, M1-E1462, M1-D1461, M1-L1460, M1-V1459, M1-L1458, M1-I1457, M1-K1456, M1-S1455, M1-K1454, M1-R1453, M1-L1452, M1-L1451, M1-A1450, M1-R1449, M1-G1448, M1-L1447, M1-C1446, M1-L1445, M1-L1444, M1-Q1443, M1-R1442, M1-Q1441, M1-G1440, M1-I1439, M1-S1438, M1-L1437, M1-N1436, M1-G1435, M1-G1434, M1-A1433, M1-E1432, M1-T1431, M1-V1430, M1-E1429, M1-H1428, M1-S1427, M1-L1426, M1-G1425, M1-L1424, M1-Q1423, M1-L1422, M1-S1421, M1-A1420, M1-V1419, M1-F1418, M1-S1417, M1-K1416, M1-L1415, M1-H1414, M1-A1413, M1-L1412, M1-E1411, M1-L1410, M1-A1409, M1-K1408, M1-W1407, M1-I1406, M1-E1405, M1-E1404, M1-D1403, M1-S1402, M1-Y1401, M1-N1400, M1-N1399, M1-F1398, M1-P1397, M1-D1396, M1-L1395, M1-N1394, M1-M1393, M1-R1392, M1-L1391, M1-S1390, M1-G1389, M1-S1388, M1-F1387, M1-L1386, M1-I1385, M1-P1384, M1-D1383, M1-Q1382, M1-P1381, M1-I1380, M1-I1379, M1-T1378, M1-L1377, M1-K1376, M1-E1375, M1-R1374, M1-L1373, M1-D1372, M1-H1371, M1-L1370, M1-G1369, M1-I1368, M1-S1367, M1-A1366, M1-I1365, M1-D1364, M1-V1363, M1-G1362, M1-D1361, M1-I1360, M1-I1359, M1-I1358, M1-Q1357, M1-G1356, M1-G1355, M1-A1354, M1-A1353, M1-E1352, M1-L1351, M1-I1350, M1-R1349, M1-F1348, M1-L1347, M1-C1346, M1-N1345, M1-T1344, M1-L1343, M1-S1342, M1-S1341, M1-K1340, M1-G1339, M1-A1338, M1-G1337, M1-T1336, M1-R1335, M1-G1334, M1-V1333, M1-V1332, M1-G1331, M1-I1330, M1-K1329, M1-E1328, M1-M1327, M1-S1326, M1-G1325, M1-I1324, M1-D1323, M1-C1322, M1-T1321, M1-I1320, M1-G1319, M1-R1318, M1-L1317, M1-V1316, M1-L1315, M1-D1314, M1-L1313, M1-E1312, M1-P1311, M1-R1310, M1-Y1309, M1-R1308, M1-V1307, M1-Q1306, M1-Y1305, M1-N1304, M1-N1303, M1-F1302, M1-Q1301, M1-I1300, M1-K1299, M1-G1298, M1-K1297, M1-S1296, M1-P1295, M1-W1294, M1-D1293, M1-P1292, M1-P1291, M1-P1290, M1-R1289, M1-K1288, M1-D1287, M1-T1286, M1-V1285, M1-W1284, M1-P1283, M1-A1282, M1-E1281, M1-N1280, M1-E1279, M1-V1278, M1-K1277, M1-T1276, M1-Y1275, M1-E1274, M1-T1273, M1-I1272, M1-R1271, M1-E1270, M1-V1269, M1-A1268, M1-V1267, M1-I1266, M1-N1265, M1-T1264, M1-E1263, M1-I1262, M1-E1261, M1-S1260, M1-T1259, M1-M1258, M1-R1257, M1-V1256, M1-L1255, M1-W1254, M1-N1253, M1-L1252, M1-T1251, M1-Q1250, M1-T1249, M1-I1248, M1-N1247, M1-L1246, M1-A1245, M1-N1244, M1-S1243, M1-L1242, M1-V1241, M1-F1240, M1-G1239, M1-V1238, M1-T1237, M1-D1236, M1-G1235, M1-S1234, M1-L1233, M1-T1232, M1-D1231, M1-R1230, M1-Y1229, M1-I1228, M1-V1227, M1-M1226, M1-M1225, M1-L1224, M1-A1223, M1-S1222, M1-F1221, M1-F1220, M1-V1219, M1-T1218, M1-L1217, M1-N1216, M1-G1215, M1-V1214, M1-L1213, M1-E1212, M1-L1211, M1-R1210, M1-I1209, M1-A1208, M1-L1207, M1-W1206, M1-R1205, M1-N1204, M1-S1203, M1-T1202, M1-I1201, M1-W1200, M1-S1199, M1-F1198, M1-V1197, M1-C1196, M1-K1195, M1-Q1194, M1-N1193, M1-T1192, M1-D1191, M1-I1190, M1-R1189, M1-E1188, M1-E1187, M1-N1186, M1-H1185, M1-K1184, M1-L1183, M1-F1182, M1-R1181, M1-Q1180, M1-Q1179, M1-H1178, M1-E1177, M1-F1176, M1-A1175, M1-R1174, M1-I1173, M1-V1172, M1-P1171, M1-L1170, M1-G1169, M1-S1168, M1-V1167, M1-T1166, M1-E1165, M1-S1164, M1-F1163, M1-H1162, M1-S1161, M1-Y1160, M1-I1159, M1-P1158, M1-S1157, M1-R1156, M1-T1155, M1-V1154, M1-S1153, M1-D1152, M1-L1151, M1-R1150, M1-R1149, M1-L1148, M1-Q1147, M1-R1146, M1-S1145, M1-T1144, M1-S1143, M1-V1142, M1-Y1141, M1-F1140, M1-M1139, M1-Q1138, M1-V1137, M1-S1136, M1-V1135, M1-Y1134, M1-I1133, M1-I1132, M1-G1131, M1-L1130, M1-P1129, M1-I1128, M1-V1127, M1-I1126, M1-I1125, M1-T1124, M1-F1123, M1-V1122, M1-P1121, M1-T1120, M1-A1119, M1-M1118, M1-C1117, M1-I1116, M1-M1115, M1-V1114, M1-L1113, M1-T1112, M1-S1111, M1-I1110, M1-I1109, M1-G1108, M1-L1107, M1-F1106, M1-C1105, M1-T1104, M1-I1103, M1-W1102, M1-S1101, M1-R1110, M1-L1099, M1-S1098, M1-Q1097, M1-P1096, M1-L1095, M1-T1094, M1-D1093, M1-D1092, M1-V1091, M1-T1090, M1-S1089, M1-I1088, M1-D1087, M1-G1086, M1-A1085, M1-F1084, M1-R1083, M1-N1082, M1-V1081, M1-I1080, M1-R1079, M1-G1078, M1-T1077, M1-P1076, M1-T1075, M1-T1074, M1-D1073, M1-F1072, M1-F1071, M1-R1070, M1-M1069, M1-P1068, M1-A1067, M1-R1066, M1-L1065, M1-I1064, M1-N1063, M1-N1062, M1-L1061, M1-L1060, M1-Q1059, M1-K1058, M1-H1057, M1-L1056, M1-I1055, M1-N1054, M1-S1053, M1-A1052, M1-H1051, M1-V1050, M1-F1049, M1-G1048, M1-F1047, M1-A1046, M1-S1045, M1-W1044, M1-F1043, M1-H1042, M1-A1041, M1-I1040, M1-F1039, M1-V1038, M1-F1037, M1-I1036, M1-G1035, M1-Q1034, M1-A1033, M1-L1032, M1-G1031, M1-L1030, M1-A1029, M1-G1028, M1-Y1027, M1-V1026, M1-G1025, M1-V1024, M1-R1023, M1-M1022, M1-D1021, M1-R1020, M1-Q1019, M1-S1018, M1-A1017, M1-P1016, M1-Y1015, M1-D1014, M1-T1013, M1-S1012, M1-N1011, M1-F1010, M1-I1009, M1-K1008, M1-S1007, M1-D1006, M1-S1005, M1-T1004, M1-W1003, M1-A1002, M1-S1001, M1-L1000, M1-W999, M1-L998, M1-N997, M1-S996, M1-G995, M1-I994, M1-F993, M1-A992, M1-V991, M1-S990, M1-N989, M1-M988, M1-V987, M1-F986, M1-A985, M1-A985, M1-L984, M1-I983, M1-I982, M1-F981, M1-F980, M1-I979, M1-S978-F977, M1-L976, M1-G975, M1-I974, M1-A973, M1-Q972, M1-L971, M1-Y970, M1-E969, M1-L968, M1-Y967, M1-I966, M1-S965, M1-F964, M1-K963, M1-V962, M1-K961, M1-G960, M1-T959, M1-E958, M1-I957, M1-F956, M1-E955, M1-K954, M1-K953, M1-I952, M1-L951, M1-K950, M1-Q949, M1-G948, M1-K947, M1-V946, M1-L945, M1-E944, M1-E943, M1-D942, M1-E941, M1-K940, M1-L939, M1-S938, M1-N937, M1-V936, M1-N935, M1-R934, M1-T933, M1-K932, M1-L931, M1-S930, M1-N929, M1-R928, M1-L927, M1-S926, M1-K925, M1-L924, M1-H923, M1-R922, M1-G921, M1-N920, M1-S919, M1-R918, M1-S917, M1-S916, M1-R915, M1-S914, M1-L913, M1-T912, M1-R911, M1-R910, M1-F909, M1-S908, M1-N907, M1-E906, M1-R905, M1-R904, M1-M903, M1-T902, M1-I901-S900, M1-A899, M1-A898, M1-D897, M1-E896, M1-P895, M1-I894, M1-E893, M892, M1-V891, M1-S890, M1-S889, M1-I888, M1-L887, M1-G886, M1-Y885, D884, M1-D883, M1-D882, M1-E881, M1-E880, M1-E879, M1-S878, M1-G877, M1-D876, M1-H875, M1-V874, M1-T873, M1-A872, M1-E871, M1-E870, M1-E869, M1-P868, M1-G867, M1-T866, M1-H865, M1-R864, M1-L863, M1-F862, M1-T861, M1-K860, M1-L859, M1-N858, M1-K857, M1-A856, M1-F855, M1-E854, M1-G853, M1-K852, M1-K851, M1-A850, M1-L849, M1-L848, M1-A847, M1-S846, M1-Y845, M1-S844, M1-G843, M1-K842, M1-E841, M1-V840, M1-I839, M1-T838, M1-G837, M1-N836, M1-G835, M1-L834, M1-V833, M1-V832, M1-I831, M1-E830, M1-D829, M1-V828, M1-Q827, M1-P826, M1-L825, M1-F824, M1-H823, M1-M822, M1-S821, M1-H820, M1-T819, M1-V818, M1-L817, M1-L816, M1-R815, M1-T814, M1-K813, M1-G812, M1-K811, M1-L810, M1-L809, M1-G808, M1-N807, M1-P806, M1-G805, M1-L804, M1-V803, M1-K802, M1-N801, M1-F800, M1-I799, M1-H798, M1-K797, M1-G796, M1-V795, M1-H794, M1-A793, M1-D792, M1-V791, M1-A790, M1-S789, M1-L788, M1-P787, M1-D786, M1-D785, M1-L784, M1-L783, M1-Y782, M1-I781, M1-D780, M1-L779, M1-N778, M1-Q777, M1-Y776, M1-T775, M1-A774, M1-R773, M1-A772, M1-L771, M1-S770, M1-I769, M1-R768, M1-Q767, M1-K766, M1-Q765, M1-G764, M1-G763, M1-S762, M1-L761, M1-N760, M1-I759, M1-G758, M1-K757, M1-E756, M1-G755, M1-I754, M1-E753, M1-A752, M1-L751, M1-D750, M1-G749, M1-G748, M1-P747, M1-L746, M1-M745, M1-E744, M1-L743, M1-D742, M1-P741, M1-L740, M1-L739, M1-A738, M1-C737, M1-A736, M1-E735, M1-L734, M1-V733, M1-Q732, M1-Q731, M1-Y730, M1-R729, M1-K728, M1-E727, M1-N726, M1-F725, M1-E724, M1-T723, M1-G722, M1-F721, M1-L720, M1-I719, M1-N718, M1-D717, M1-K716, M1-I715, M1-T714, M1-G713, M1-N712, M1-Q711, M1-I710, M1-W709, M1-S708, M1-Q707, M1-Q706, M1-P705, M1-V704, M1-Y703, M1-A702, M1-T701, M1-T700, M1-G699, M1-K698, M1-I697, M1-T696, M1-I695, M1-H694, M1-G693, M1-H692, M1-V691, M1-N690, M1-E689, M1-M688, M1-E687, M1-G686, M1-L685, M1-M684, M1-A683, M1-S682, M1-I681, M1-L680, M1-S679, M1-S678, M1-K677, M1-G676, M1-S675, M1-G674, M1-V673, M1-P672, M1-G671, M1-I670, M1-V669, M1-A668, M1-V667, M1-L666, M1-Q665, M1-G664, M1-A663, M1-M662, M1-I661, M1-D660, M1-L659, M1-N658, M1-V657, M1-D656, M1-R655, M1-V654, M1-T653, M1-A652, M1-E651, M1-S650, M1-D649, M1-H648, M1-E647, M1-W646, M1-T645, M1-F644, M1-S643, M1-A642, M1-E641, M1-S640, M1-F639, M1-Q638, M1-M637, M1-A636, M1-K635, M1-D634, M1-F633, M1-N632, M1-C631, M1-D630, M1-H629, M1-R628, M1-I627, M1-A626, M1-S625, M1-T624, M1-D623, M1-L622, M1-D621, M1-D620, M1-G619, M1-G618, M1-L617, M1-Y616, M1-K615, M1-E614, M1-L613, M1-R612, M1-E611, M1-T610, M1-S609, M1-V608, M1-S607, M1-A606, M1-Q605, M1-L604, M1-M603, M1-S602, M1-S601, M1-I600, M1-M599, M1-M598, M1-P597, M1-L596, M1-M595, M1-S594, M1-L593, M1-P592, M1-F591, M1-R590, M1-L589, M1-I588, M1-N587, M1-F586, M1-L585, M1-T584, M1-I583, M1-S582, M1-T581, M1-F580, M1-A579, M1-K578, M1-Q577, M1-A576, M1-D575, M1-L574, M1-I573, M1-N572, M1-N571, M1-S570, M1-D569, M1-V568, M1-L567, M1-V566, M1-Y565, M1-V564, M1-S563, M1-F562, M1-T561, M1-V560, M1-V559, M1-S558, M1-V557, M1-L556, M1-V555, M1-P554, M1-T553, M1-L552, M1-Q551, M1-F550, M1-V549, M1-F548, M1-I547, M 1 -V546, M1-V545, M1-C544, M1-Q543, M1-L542, M1-Q541, M1-S540, M1-F539, M1-A538, M1-L537, M1-L536, M1-N535, M1-K534, M1-L533, M1-E532, M1-K531, M1-K530, M1-R529, M1-L528, M1-N527, M1-Q526, M1-V525, M1-Q524, M1-D523, M1-R522, M1-F521, M1-S520, M1-P519, M1-E518, M1-W517, M1-A516, M1-F515, M1-Y514, M1-K513, M1-L512, M1-I511, M1-K510, M1-I509, M1-G508, M1-S507, M1-L506, M1-I505, M1-E504, M1-N503, M1-M502, M1-I501, M1-K500, M1-L499, M1-R498, M1-K497, M1-D496, M1-K495, M1-N494, M1-K493, M1-M492, M1-N491, M1-K490, M1-V489, M1-Q488, M1-I487, M1-T486, M1-K485, M1-S484, M1-K483, M1-T482, M1-S481, M1-L480, M1-I479, M1-A478, M1-N477, M1-I476, M1-P475, M1-I474, M1-V473, M1-L472, M1-V471, M1-M470, M1-V469, M1-G468, M1-V467, M1-G466, M1-A465, M1-L464, M1-V463, M1-S462, M1-P461, M1-G460, M1-L459, M1-E458, M1-R457, M1-W456, M1-L455, M1-F454, M1-F453, M1-I452, M1-S451, M1-LA450, M1-V449, M1-I448, M1-Q447, M1-L446, M1-V445, M1-S444, M1-S443, M1-W442, M1-L441, M1-M440, M1-H439, M1-M438, M1-F437, M1-N436, M1-T435, M1-V434, M1-D433, M1-M432, M1-L431, M1-K430, M1-Q429, M1-A428, M1-D427, M1-V426, M1-S425, M1-M424, M1-L423, M1-N422, M1-V421, M1-T420, M1-E419, M1-G418, M1-V417, M1-T416, M1-Y415, M1-E414, M1-K413, M1-R412, M1-A411, M1-L410, M1-N409, M1-S408, and/or M1-K407 of SEQ ID NO:46. Polynucleotide sequences encoding these polypeptides are also provided. The present invention also encompasses the use of these C-terminal cMOAT (SNP_ID:PS101s24) deletion polypeptides as immunogenic and/or antigenic epitopes as described elsewhere herein.

Alternatively, preferred polypeptides of the present invention may comprise polypeptide sequences corresponding to, for example, internal regions of the cMOAT (SNP_ID: PS101s24) polypeptide (e.g., any combination of both N- and C-terminal cMOAT (SNP_ID: PS101s24) polypeptide deletions) of SEQ ID NO:46. For example, internal regions could be defined by the equation: amino acid NX to amino acid CX, wherein NX refers to any N-terminal deletion polypeptide amino acid of cMOAT (SNP_ID: PS101s24) (SEQ ID NO:46), and where CX refers to any C-terminal deletion polypeptide amino acid of cMOAT (SNP_ID: PS101s24) (SEQ ID NO:46). Polynucleotides encoding these polypeptides are also provided. The present invention also encompasses the use of these polypeptides as an immunogenic and/or antigenic epitope as described elsewhere herein. Preferably, the resulting deletion polypeptide comprises the polypeptide polymorphic loci identified elsewhere herein for cMOAT (SNP_ID: PS101s24), and more preferably comprises the polypeptide polymorphic allele identified elsewhere herein for cMOAT (SNP_ID: PS101s24).

### Features of the Polypeptide Encoded by Gene No:22

The present invention relates to isolated nucleic acid molecules comprising, or alternatively, consisting of all or a portion of the variant allele of the human cMOAT, ATP-binding cassette sub-family C member 2 gene (SNP_ID: PS101s32) (e.g., wherein reference or wildtype cMOAT gene is exemplified by SEQ ID NO:1). Preferred portions are at least 10, preferably at least 20, preferably at least 40, preferably at least 100, contiguous polynucleotides and comprise an “A” at the nucleotide position corresponding to nucleotide 3035 of the cMOAT gene, or a portion of SEQ ID NO:47. Alternatively, preferred portions are at least 10, preferably at least 20, preferably at least 40, preferably at least 100, contiguous polynucleotides and comprise a “G” at the nucleotide position corresponding to nucleotide 3035 of the cMOAT gene, or a portion of SEQ ID NO:47. The invention further relates to isolated gene products, e.g., polypeptides and/or proteins, which are encoded by a nucleic acid molecule comprising all or a portion of the variant allele of the cMOAT gene.

In one embodiment, the invention relates to a method for predicting the likelihood that an individual will have a disorder associated with an “A” at the nucleotide position corresponding to nucleotide position 3035 of SEQ ID NO:47 (or diagnosing or aiding in the diagnosis of such a disorder) comprising the steps of obtaining a DNA sample from an individual to be assessed and determining the nucleotide present at position 3035 of SEQ ID NO:47. The presence of an “A” at this position indicates that the individual has a greater likelihood of having a disorder associated therewith than an individual having a “G” at that position, or a greater likelihood of having more severe symptoms.

Conversely, the invention relates to a method for predicting the likelihood that an individual will have a disorder associated with a “G” at the nucleotide position corresponding to nucleotide position 3035 of SEQ ID NO:47 (or diagnosing or aiding in the diagnosis of such a disorder) comprising the steps of obtaining a DNA sample from an individual to be assessed and determining the nucleotide present at position 3035 of SEQ ID NO:47. The presence of a “G” at this position indicates that the individual has a greater likelihood of having a disorder associated therewith than an individual having an “A” at that position, or a greater likelihood of having more severe symptoms.

Representative disorders which may be detected, diagnosed, identified, treated, prevented, and/or ameliorated by the present invention include, the following, non-limiting diseases and disorders: decreased heptic uptake of administered statins, resistance to beneficial responses of administered statins; resistance to LDL lowering responses to administered statins; resistance to TG lowering responses to administered statins; resistance to HDL elevating responses to administered statins; increased incidence of drug interactions between one or more administered statins; increased incidence of drug and endogenous substance interactions between a statin drug and endogenous substrates of cMOAT including cholate, taurocholate, thyroid hormones T3 and T4, DHEAS, estradiol-17beta-glucuronide, estrone-3-sulfate, prostaglandin E2, thromboxane B2, leukotriene C4, leukotriene E4, and bilirubin and its conjugates; decreased response to HMG-CoA reductase inhibitors; in addition to other disorders referenced herein, which include, for example, any disorder related to high levels of circulating LDL, high levels of TG, and/or low levels of circularing HDL, which include, but are not limited to, cardiovascular diseases, hepatic diseases, angina pectoris, hypertension, heart failure, myocardial infarction, ventricular hypertrophy, vascular diseases, miscrovascular disease, vascular leak syndrome, aneurysm, stroke, embolism, thrombosis, endothelial dysfunction, coronary artery disease, arteriosclerosis, and/or atherosclerosis.

TABLE I

| | | | | NT | Total | | | AA | |
|---|---|---|---|---|---|---|---|---|---|
| | | | | SEQ | NT | 5′ NT | | Seq | Total |
| | CDNA | NT | AA | ID. | Seq | of Start | 3′ NT | ID | AA |
| Gene | Name/ | Poly- | Poly- | No. | of | Codon | of | No. | of |
| No. | SNP_ID | morphism | morphism | X | Clone | of ORF | of ORF | Y | ORF |
| 1. | OATP2/ | G545A | N/A | 5 | 2830 | 135 | 2210 | 6 | 691 |
| | PS100s1 | | | | | | | | |
| 2. | OATP2/ | C597A | P155T | 7 | 2830 | 135 | 2210 | 8 | 691 |
| | PS100s2 | | | | | | | | |
| 3. | OATP2/ | G522T | D130Y | 9 | 2830 | 135 | 2210 | 10 | 691 |
| | PS100s9 | | | | | | | | |
| 4. | OATP2/ | G1597C | G488A | 11 | 2830 | 135 | 2210 | 12 | 691 |
| | PS100s23 | | | | | | | | |
| 5. | OATP2/ | G1382A | N/A | 13 | 2830 | 135 | 2210 | 14 | 691 |
| | PS100s25 | | | | | | | | |
| 6. | OATP2/ | C1334G | F400K | 15 | 2830 | 135 | 2210 | 16 | 691 |
| | PS100s26 | | | | | | | | |
| 7. | OATP2/ | T655C | V174A | 17 | 2830 | 135 | 2210 | 18 | 691 |
| | PS100s29 | | | | | | | | |
| 8. | OATP2/ | T705C | K191L | 19 | 2830 | 135 | 2210 | 20 | 691 |
| | PS100s30 | | | | | | | | |
| 9. | OATP2/ | C731T | N/A | 21 | 2830 | 135 | 2210 | 22 | 691 |
| | PS100s31 | | | | | | | | |
| 10. | CMOAT/ | A3664T | E1188V | 23 | 5300 | 102 | 4739 | 24 | 1545 |
| | PS101s1 | | | | | | | | |
| 11. | CMOAT/ | C4073T | N/A | 25 | 5300 | 102 | 4739 | 26 | 1545 |
| | PS101s2 | | | | | | | | |
| 12. | CMOAT/ | C4211T | N/A | 27 | 5300 | 102 | 4739 | 28 | 1545 |
| | PS101s4 | | | | | | | | |
| 13. | CMOAT/ | C4163T | N/A | 29 | 5300 | 102 | 4739 | 30 | 1545 |
| | PS101s5 | | | | | | | | |
| 14. | CMOAT/ | G4511A | N/A | 31 | 5300 | 102 | 4739 | 32 | 1545 |
| | PS101s6 | | | | | | | | |
| 15. | CMOAT/ | T4589C | N/A | 33 | 5300 | 102 | 4739 | 34 | 1545 |
| | PS101s7 | | | | | | | | |
| 16. | CMOAT/ | G3643T | R1181L | 35 | 5300 | 102 | 4739 | 36 | 1545 |
| | PS101s10 | | | | | | | | |
| 17. | CMOAT/ | A2983G | K961R | 37 | 5300 | 102 | 4739 | 38 | 1545 |
| | PS101S11 | | | | | | | | |
| 18. | CMOAT/ | A359G | N/A | 39 | 5300 | 102 | 4739 | 40 | 1545 |
| | PS101s13 | | | | | | | | |
| 19. | CMOAT/ | T2110C | 1670T | 41 | 5300 | 102 | 4739 | 42 | 1545 |
| | PS101s22 | | | | | | | | |
| 20. | CMOAT/ | G1350A | V4171 | 43 | 5300 | 102 | 4739 | 44 | 1545 |
| | PS101s23 | | | | | | | | |
| 21. | CMOAT/ | C1320T | L407K | 45 | 5300 | 102 | 4739 | 46 | 1545 |
| | PS101s24 | | | | | | | | |
| 22. | CMOAT/ | G3035A | N/A | 47 | 5300 | 102 | 4739 | 48 | 1545 |
| | PS101s32 | | | | | | | | |

Table I summarizes the information corresponding to each “Gene No.” described above. The nucleotide sequence identified as “NT SEQ ID NO:X” refers to the complete cDNA of the nucleotide comprising at least one polymorphism of the present invention and was identified using the methods described elsewhere herein, resulting in a final sequence identified as SEQ ID NO:X.

“cDNA Name/SNP_ID” refers to the accepted name of the wild type gene according to the HUGO Gene Nomenclature Committee, while the “SNP_ID” identifies the novel polymorphism provided as described in Tables IV, V, and VI, and the Examples herein. The SNP_ID uniquely identifies the novel SNPs of the present invention, and likewise the novel polynucleotide and polypeptides of the present invention which comprise these SNPs. The inclusion of the cDNA Name is provided for reference.

“NT Polymorphism” describes the specific nucleotide location within the coding region of each polynucleotide sequence of the present invention, in addition to the reference and variable nucleotides at that position. The format of this designation is as follows: R—N-A, where “N” refers to the nucleotide position of the polymorphism as shown in the Sequence Listing and/or Figures herein, the nucleotide provided in the “R” position refers to the reference nucleotide at the “N” position, while the nucleotide provided in the “A” position refers to the variable nucleotide at the “N” position.

“AA Polymorphism” describes the specific amino acid location within the encoded polypeptide sequence of the present invention, in addition to the reference and variable amino acids at that position. The format of this designation is as follows: R—N-A, where “N” refers to the amino acid position of the encoded polymorphism as shown in the Sequence Listing and/or Figures herein, the amino acid provided in the “R” position refers to the reference amino acid at the “N” position, while the amino acid provided in the “A” position refers to the variable amino acid at the “N” position.

“Total NT Seq. Of Clone” refers to the total number of nucleotides in the clone identified by “Gene No.” The nucleotide position of SEQ ID NO:X of the putative start codon (methionine) is identified as “5+ NT of Start Codon of ORF.”

The translated amino acid sequence, beginning with the methionine, is identified as “AA SEQ ID NO:Y” although other reading frames can also be easily translated using known molecular biology techniques. The polypeptides produced by these alternative open reading frames are specifically contemplated by the present invention.

The total number of amino acids within the open reading frame of SEQ ID NO:6, 8, 10, 12, 14, 16, 18, 20, 22, 24, 26, 28, 30, 32, 34, 36, 38, 40, 42, 44, 46, 48, 50, and/or 604 is identified as “Total AA of ORF”.

SEQ ID NO:X (where X may be any of the polynucleotide sequences disclosed in the sequence listing) and the translated SEQ ID NO:Y (where Y may be any of the polypeptide sequences disclosed in the sequence listing) are sufficiently accurate and otherwise suitable for a variety of uses well known in the art and described further herein. For instance, SEQ ID NO:5, 7, 9, 11, 13, 15, 17, 19, 21, 23, 25, 27, 29, 31, 33, 35, 37, 39, 41, 43, 45, 47, 49, and/or 603 is useful for designing nucleic acid hybridization probes that will detect nucleic acid sequences contained in SEQ ID NO:5, 7, 9, 11, 13, 15, 17, 19, 21, 23, 25, 27, 29, 31, 33, 35, 37, 39, 41, 43, 45, 47, 49, and/or 603. These probes will also hybridize to nucleic acid molecules in biological samples, thereby enabling a variety of forensic and diagnostic methods of the invention. Similarly, polypeptides identified from SEQ ID NO:6, 8, 10, 12, 14, 16, 18, 20, 22, 24, 26, 28, 30, 32, 34, 36, 38, 40, 42, 44, 46, 48, 50, and/or 604 may be used, for example, to generate antibodies which bind specifically to proteins containing the polypeptides and the proteins encoded by the cDNA clones identified in Table I.

Nevertheless, DNA sequences generated by sequencing reactions can contain sequencing errors. The errors exist as misidentified nucleotides, or as insertions or deletions of nucleotides in the generated DNA sequence. The erroneously inserted or deleted nucleotides may cause frame shifts in the reading frames of the predicted amino acid sequence. In these cases, the predicted amino acid sequence diverges from the actual amino acid sequence, even though the generated DNA sequence may be greater than 99.9% identical to the actual DNA sequence (for example, one base insertion or deletion in an open reading frame of over 1000 bases).

Accordingly, for those applications requiring precision in the nucleotide sequence or the amino acid sequence, the present invention provides the generated nucleotide sequence identified as SEQ ID NO:5, 7, 9, 11, 13, 15, 17, 19, 21, 23, 25, 27, 29, 31, 33, 35, 37, 39, 41, 43, 45, 47, 49, and/or 603 and the predicted translated amino acid sequence identified as SEQ ID NO:6, 8, 10, 12, 14, 16, 18, 20, 22, 24, 26, 28, 30, 32, 34, 36, 38, 40, 42, 44, 46, 48, 50, and/or 604, as set forth in Table I. Moreover, the amino acid sequence of the protein encoded by a particular clone can also be directly determined by peptide sequencing or by collecting the protein, and determining its sequence.

The present invention also relates to the genes corresponding to SEQ ID NO:5, 7, 9, 11, 13, 15, 17, 19, 21, 23, 25, 27, 29, 31, 33, 35, 37, 39, 41, 43, 45, 47, 49, and/or 603, SEQ ID NO:6, 8, 10, 12, 14, 16, 18, 20, 22, 24, 26, 28, 30, 32, 34, 36, 38, 40, 42, 44, 46, 48, 50, and/or 604. The corresponding gene can be isolated in accordance with known methods using the sequence information disclosed herein. Such methods include preparing probes or primers from the disclosed sequence and identifying or amplifying the corresponding gene from appropriate sources of genomic material.

Also provided in the present invention are species homologs, allelic variants, and/or orthologs. The skilled artisan could, using procedures well-known in the art, obtain the polynucleotide sequence corresponding to full-length genes (including, but not limited to the full-length coding region), allelic variants, splice variants, orthologs, and/or species homologues of genes corresponding to SEQ ID NO:5, 7, 9, 11, 13, 15, 17, 19, 21, 23, 25, 27, 29, 31, 33, 35, 37, 39, 41, 43, 45, 47, 49, and/or 603, SEQ ID NO:6, 8, 10, 12, 14, 16, 18, 20, 22, 24, 26, 28, 30, 32, 34, 36, 38, 40, 42, 44, 46, 48, 50, and/or 604. For example, allelic variants and/or species homologues may be isolated and identified by making suitable probes or primers which correspond to the 5′, 3′, or internal regions of the sequences provided herein and screening a suitable nucleic acid source for allelic variants and/or the desired homologue.

The polypeptides of the invention can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

The polypeptides may be in the form of the protein, or may be a part of a larger protein, such as a fusion protein (see below). It is often advantageous to include an additional amino acid sequence which contains secretory or leader sequences, pro-sequences, sequences which aid in purification, such as multiple histidine residues, or an additional sequence for stability during recombinant production.

The polypeptides of the present invention are preferably provided in an isolated form, and preferably are substantially purified. A recombinantly produced version of a polypeptide, can be substantially purified using techniques described herein or otherwise known in the art, such as, for example, by the one-step method described in Smith and Johnson, Gene 67:31-40 (1988). Polypeptides of the invention also can be purified from natural, synthetic or recombinant sources using protocols described herein or otherwise known in the art, such as, for example, antibodies of the invention raised against the full-length form of the protein.

The present invention provides a polynucleotide comprising, or alternatively consisting of, the sequence identified as SEQ ID NO:5, 7, 9, 11, 13, 15, 17, 19, 21, 23, 25, 27, 29, 31, 33, 35, 37, 39, 41, 43, 45, 47, 49, and/or 603. The present invention also provides a polypeptide comprising, or alternatively consisting of, the sequence identified as SEQ ID NO:6, 8, 10, 12, 14, 16, 18, 20, 22, 24, 26, 28, 30, 32, 34, 36, 38, 40, 42, 44, 46, 48, 50, and/or 604. The present invention also provides polynucleotides encoding a polypeptide comprising, or alternatively consisting of the polypeptide sequence of SEQ ID NO:6, 8, 10, 12, 14, 16, 18, 20, 22, 24, 26, 28, 30, 32, 34, 36, 38, 40, 42, 44, 46, 48, 50, and/or 604.

Preferably, the present invention is directed to a polynucleotide comprising, or alternatively consisting of, the sequence identified as SEQ ID NO:5, 7, 9, 11, 13, 15, 17, 19, 21, 23, 25, 27, 29, 31, 33, 35, 37, 39, 41, 43, 45, 47, 49, and/or 603, that is less than, or equal to, a polynucleotide sequence that is 5 mega basepairs, 1 mega basepairs, 0.5 mega basepairs, 0.1 mega basepairs, 50,000 basepairs, 20,000 basepairs, or 10,000 basepairs in length.

The present invention encompasses polynucleotides with sequences complementary to those of the polynucleotides of the present invention disclosed herein. Such sequences may be complementary to the sequence disclosed as SEQ ID NO:5, 7,9, 11, 13, 15, 17, 19, 21, 23, 25, 27, 29, 31, 33, 35, 37, 39, 41, 43, 45, 47, 49, and/or 603, and/or the nucleic acid sequence encoding the sequence disclosed as SEQ ID NO:6, 8, 10, 12, 14, 16, 18, 20, 22, 24, 26, 28, 30, 32, 34, 36, 38, 40, 42, 44, 46, 48, 50, and/or 604.

The present invention also encompasses polynucleotides capable of hybridizing, preferably under reduced stringency conditions, more preferably under stringent conditions, and most preferably under highly stringent conditions, to polynucleotides described herein. Examples of stringency conditions are shown in Table II below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE II

| | | | Hybridization | Wash |
|---|---|---|---|---|
| | Polynucleotide | Hybrid | Temperature | Temperature |
| Stringency Condition | Hybrid± | Length (bp)‡ | and Buffer† | and Buffer† |
| A | DNA:DNA | > or equal to | 65° C.; 1 × SSC - | 65° C.; |
| | | 50 | or- 42° C.; | 0.3 × SSC |
| | | | 1 × SSC, 50% | |
| | | | formamide | |
| B | DNA:DNA | <50 | Tb*; 1 × SSC | Tb*; 1 × SSC |
| C | DNA:RNA | > or equal to | 67° C.; 1 × SSC - | 67° C.; |
| | | 50 | or- 45° C.; | 0.3 × SSC |
| | | | 1 × SSC, 50% | |
| | | | formamide | |
| D | DNA:RNA | <50 | Td*; 1 × SSC | Td*; 1 × SSC |
| E | RNA:RNA | > or equal to | 70° C.; 1 × SSC - | 70° C.; |
| | | 50 | or- 50° C.; | 0.3 × SSC |
| | | | 1 × SSC, 50% | |
| | | | formamide | |
| F | RNA:RNA | <50 | Tf*; 1 × SSC | Tf*; 1 × SSC |
| G | DNA:DNA | > or equal to | 65° C.; 4 × SSC - | 65° C.; 1 × SSC |
| | | 50 | or- 45° C.; | |
| | | | 4 × SSC, 50% | |
| | | | formamide | |
| H | DNA:DNA | <50 | Th*; 4 × SSC | Th*; 4 × SSC |
| I | DNA:RNA | > or equal to | 67° C.; 4 × SSC - | 67° C.; 1 × SSC |
| | | 50 | or- 45° C.; | |
| | | | 4 × SSC, 50% | |
| | | | formamide | |
| J | DNA:RNA | <50 | Tj*; 4 × SSC | Tj*; 4 × SSC |
| K | RNA:RNA | > or equal to | 70° C.; 4 × SSC - | 67° C.; 1 × SSC |
| | | 50 | or- 40° C.; | |
| | | | 6 × SSC, 50% | |
| | | | formamide | |
| L | RNA:RNA | <50 | Tl*; 2 × SSC | Tl*; 2 × SSC |
| M | DNA:DNA | > or equal to | 50° C.; 4 × SSC - | 50° C.; 2 × SSC |
| | | 50 | or- 40° C. | |
| | | | 6 × SSC, 50% | |
| | | | formamide | |
| N | DNA:DNA | <50 | Tn*; 6 × SSC | Tn*; 6 × SSC |
| O | DNA:RNA | > or equal to | 55° C.; 4 × SSC - | 55° C.; 2 × SSC |
| | | 50 | or- 42° C.; | |
| | | | 6 × SSC, 50% | |
| | | | formamide | |
| P | DNA:RNA | <50 | Tp*; 6 × SSC | Tp*; 6 × SSC |
| Q | RNA:RNA | > or equal to | 60° C.; 4 × SSC - | 60° C.; 2 × SSC |
| | | 50 | or- 45° C.; | |
| | | | 6 × SSC, 50% | |
| | | | formamide | |
| R | RNA:RNA | <50 | Tr*; 4 × SSC | Tr*; 4 × SSC |

‡—The “hybrid length” is the anticipated length for the hybridized region(s) of the hybridizing polynucleotides. When hybridizing a polynucleotide of unknown sequence, the hybrid is assumed to be that of the hybridizing polynucleotide of the present invention. When polynucleotides of known sequence are hybridized, the hybrid length can be determined by aligning the sequences of the polynucleotides and identifying the region of regions of optimal sequence complementarity. Methods of aligning two or more polynucleotide sequences and/or determining the percent identity between two polynucleotide sequences are well known in the art (e.g., MegAlign program of the DNA*Star suite of programs, etc).

†—SSPE (1×SSPE is 0.15M NaCl, 10 mM NaH2PO4, and 1.25 mM EDTA, pH 7.4) can be substituted for SSC (1×SSC is 0.15M NaCl and 15 mM sodium citrate) in the hybridization and wash buffers; washes are performed for 15 minutes after hybridization is complete. The hydridizations and washes may additionally include 5× Denhardt's reagent, 0.5-1.0% SDS, 100 ug/ml denatured, fragmented salmon sperm DNA, 0.5% sodium pyrophosphate, and up to 50% formamide.

*Tb-Tr: The hybridization temperature for hybrids anticipated to be less than 50 base pairs in length should be 5-10° C. less than the melting temperature Tm of the hybrids there Tm is determined according to the following equations. For hybrids less than 18 base pairs in length, Tm(° C.)=2(# of A+T bases)+4(# of G+C bases). For hybrids between 18 and 49 base pairs in length, Tm(° C.)=81.5±16.6(log₁₀[Na+])+0.41(%G+C)−(600/N), where N is the number of bases in the hybrid, and [Na+] is the concentration of sodium ions in the hybridization buffer ([NA+] for 1×SSC=0.165 M).

±—The present invention encompasses the substitution of any one, or more DNA or RNA hybrid partners with either a PNA, or a modified polynucleotide. Such modified polynucleotides are known in the art and are more particularly described elsewhere herein.

Additional examples of stringency conditions for polynucleotide hybridization are provided, for example, in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M., Ausubel et al., eds, John Wiley and Sons, Inc., sections 2.10 and 6.3-6.4, which are hereby incorporated by reference herein.

Preferably, such hybridizing polynucleotides have at least 70% sequence identity (more preferably, at least 80% identity; and most preferably at least 90% or 95% identity) with the polynucleotide of the present invention to which they hybridize, where sequence identity is determined by comparing the sequences of the hybridizing polynucleotides when aligned so as to maximize overlap and identity while minimizing sequence gaps. The determination of identity is well known in the art, and discussed more specifically elsewhere herein.

The invention encompasses the application of PCR methodology to the polynucleotide sequences of the present invention, and/or the cDNA encoding the polypeptides of the present invention. PCR techniques for the amplification of nucleic acids are described in U.S. Pat. No. 4,683,195 and Saiki et al., Science, 239:487-491 (1988). PCR, for example, may include the following steps, of denaturation of template nucleic acid (if double-stranded), annealing of primer to target, and polymerization. The nucleic acid probed or used as a template in the amplification reaction may be genomic DNA, cDNA, RNA, or a PNA. PCR may be used to amplify specific sequences from genomic DNA, specific RNA sequence, and/or cDNA transcribed from mRNA. References for the general use of PCR techniques, including specific method parameters, include Mullis et al., Cold Spring Harbor Symp. Quant. Biol., 51:263, (1987), Ehrlich (ed), PCR Technology, Stockton Press, NY, 1989; Ehrlich et al., Science, 252:1643-1650, (1991); and “PCR Protocols, A Guide to Methods and Applications”, Eds., Innis et al., Academic Press, New York, (1990).

### Polynucleotide and Polypeptide Variants

The present invention also encompasses variants (e.g., allelic variants, orthologs, etc.) of the polynucleotide sequence disclosed herein in SEQ ID NO:5, 7, 9, 11, 13, 15, 17, 19, 21, 23, 25, 27, 29, 31, 33, 35, 37, 39, 41, 43, 45, 47, 49, and/or 603, the complementary strand thereto.

The present invention also encompasses variants of the polypeptide sequence, and/or fragments therein, disclosed in SEQ ID NO:6, 8, 10, 12, 14, 16, 18, 20, 22, 24, 26, 28, 30, 32, 34, 36, 38, 40, 42, 44, 46, 48, 50, and/or 604, a polypeptide encoded by the polynucleotide sequence in SEQ ID NO:5, 7, 9, 11, 13, 15, 17, 19, 21, 23, 25, 27, 29, 31, 33, 35, 37, 39, 41, 43, 45, 47, 49, and/or 603.

“Variant” refers to a polynucleotide or polypeptide differing from the polynucleotide or polypeptide of the present invention, but retaining essential properties thereof. Generally, variants are overall closely similar, and, in many regions, identical to the polynucleotide or polypeptide of the present invention.

Thus, one aspect of the invention provides an isolated nucleic acid molecule comprising, or alternatively consisting of, a polynucleotide having a nucleotide sequence selected from the group consisting of: (a) a nucleotide sequence encoding a related polypeptide of the present invention having an amino acid sequence as shown in the sequence listing and described in SEQ ID NO:5, 7, 9, 11, 13, 15, 17, 19, 21, 23, 25, 27, 29, 31, 33, 35, 37, 39, 41, 43, 45, 47, 49, and/or 603 ; (b) a nucleotide sequence encoding a mature related polypeptide of the present invention having the amino acid sequence as shown in the sequence listing and described in SEQ ID NO:5, 7, 9, 11, 13, 15, 17, 19, 21, 23, 25, 27, 29, 31, 33, 35, 37, 39, 41, 43, 45, 47, 49, and/or 603; (c) a nucleotide sequence encoding a biologically active fragment of a related polypeptide of the present invention having an amino acid sequence shown in the sequence listing and described in SEQ ID NO:5, 7, 9, 11, 13, 15, 17, 19, 21, 23, 25, 27, 29, 31, 33, 35, 37, 39, 41, 43, 45, 47, 49, and/or 603 ; (d) a nucleotide sequence encoding an antigenic fragment of a related polypeptide of the present invention having an amino acid sequence sown in the sequence listing and described in SEQ ID NO:5, 7,9, 11, 13, 15, 17, 19, 21, 23, 25, 27, 29, 31, 33, 35, 37, 39, 41, 43, 45, 47, 49, and/or 603; (e) a nucleotide sequence encoding a related polypeptide of the present invention comprising the complete amino acid sequence encoded by a human cDNA plasmid contained in SEQ ID NO:5, 7, 9, 11, 13, 15, 17, 19, 21, 23, 25, 27, 29, 31, 33, 35, 37, 39, 41, 43, 45, 47, 49, and/or 603 ; (f) a nucleotide sequence encoding a mature related polypeptide,of the present invention having an amino acid sequence encoded by a human cDNA plasmid contained in SEQ ID NO:5, 7, 9, 11, 13, 15, 17, 19, 21, 23, 25, 27, 29, 31, 33, 35, 37, 39, 41, 43, 45, 47, 49, and/or 603 ; (g) a nucleotide sequence encoding a biologically active fragment of a related polypeptide of the present invention having an amino acid sequence encoded by a human cDNA plasmid contained in SEQ ID NO:5, 7, 9, 11, 13, 15, 17, 19, 21, 23, 25, 27, 29, 31, 33, 35, 37, 39, 41, 43, 45, 47, 49, and/or 603 ; (h) a nucleotide sequence encoding an antigenic fragment of a related polypeptide of the present invention having an amino acid sequence encoded by a human cDNA plasmid contained in SEQ ID NO:5, 7, 9, 11, 13, 15, 17, 19, 21, 23, 25, 27, 29, 31, 33, 35, 37, 39, 41, 43, 45, 47, 49, and/or 603; (I) a nucleotide sequence complimentary to any of the nucleotide sequences in (a), (b), (c), (d), (e), (f), (g), or (h), above.

The present invention is also directed to polynucleotide sequences which comprise, or alternatively consist of, a polynucleotide sequence which is at least about 80%, 85%, 90%, 91%, 92%, 93%, 94%, 95%, 96%, 97%, 98%, 99%, 99.1% 99.2% 99.3%, 99.4%, 99.5%, 99.6%, 99.7%, 99.8%, or 99.9% identical to, for example, any of the nucleotide sequences in (a), (b), (c), (d), (e), (f), (g), or (h), above. Polynucleotides encoded by these nucleic acid molecules are also encompassed by the invention. In another embodiment, the invention encompasses nucleic acid molecules which comprise, or alternatively, consist of a polynucleotide which hybridizes under stringent conditions, or alternatively, under lower stringency conditions, to a polynucleotide in (a), (b), (c), (d), (e), (f), (g), or (h), above. Polynucleotides which hybridize to the complement of these nucleic acid molecules under stringent hybridization conditions or alternatively, under lower stringency conditions, are also encompassed by the invention, as are polypeptides encoded by these polypeptides.

Another aspect of the invention provides an isolated nucleic acid molecule comprising, or alternatively, consisting of, a polynucleotide having a nucleotide sequence selected from the group consisting of: (a) a nucleotide sequence encoding a related polypeptide of the present invention having an amino acid sequence as shown in the sequence listing and described in Table I, IV, V, or VI; (b) a nucleotide sequence encoding a mature related polypeptide of the present invention having the amino acid sequence as shown in the sequence listing and described in Table I, IV, V, or VI; (c) a nucleotide sequence encoding a biologically active fragment of a related polypeptide of the present invention having an amino acid sequence as shown in the sequence listing and described in Table I, VI, V, or VI; (d) a nucleotide sequence encoding an antigenic fragment of a related polypeptide of the present invention having an amino acid sequence as shown in the sequence listing and described in Table I, IV, V, or VI; (e) a nucleotide sequence complimentary to any of the nucleotide sequences in (a), (b), (c), (d), or (e) above.

The present invention is also directed to nucleic acid molecules which comprise, or alternatively, consist of, a nucleotide sequence which is at least about 80%, 85%, 90%, 91%, 92%, 93%, 94%, 95%, 96%, 97%, 98%, 99%, 99.1% 99.2%, 99.3%, 99.4%, 99.5%, 99.6%, 99.7%, 99.8%, or 99.9% identical to, for example, any of the nucleotide sequences in (a), (b), (c), (d), or (e) above.

The present invention encompasses polypeptide sequences which comprise, or alternatively consist of, an amino acid sequence which is at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to, the following non-limited examples, the polypeptide sequence identified as SEQ ID NO:6, 8, 10, 12, 14, 16, 18, 20, 22, 24, 26, 28, 30, 32, 34, 36, 38, 40, 42, 44, 46, 48, 50, and/or 604, and/or polypeptide fragments of any of the polypeptides provided herein. Polynucleotides encoded by these nucleic acid molecules are also encompassed by the invention. In another embodiment, the invention encompasses nucleic acid molecules which comprise, or alternatively, consist of a polynucleotide which hybridizes under stringent conditions, or alternatively, under lower stringency conditions, to a polynucleotide in (a), (b), (c), (d), or (e) above. Polynucleotides which hybridize to the complement of these nucleic acid molecules under stringent hybridization conditions or alternatively, under lower stringency conditions, are also encompassed by the invention, as are polypeptides encoded by these polypeptides.

The present invention is also directed to polypeptides which comprise, or alternatively consist of, an amino acid sequence which is at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to, for example, the polypeptide sequence shown in SEQ ID NO:6, 8, 10, 12, 14, 16, 18, 20, 22, 24, 26, 28, 30, 32, 34, 36, 38, 40, 42, 44, 46, 48, 50, and/or 604, a polypeptide sequence encoded by the nucleotide sequence in SEQ ID NO:5, 7, 9, 11, 13, 15, 17, 19, 21, 23, 25, 27, 29, 31, 33, 35, 37, 39, 41, 43, 45, 47, 49, and/or 603, a polypeptide sequence encoded by the cDNA provided in Table I, and/or polypeptide fragments of any of these polypeptides (e.g., those fragments described herein). Polynucleotides which hybridize to the complement of the nucleic acid molecules encoding these polypeptides under stringent hybridization conditions or alternatively, under lower stringency conditions, are also encompasses by the present invention, as are the polypeptides encoded by these polynucleotides.

By a nucleic acid having a nucleotide sequence at least, for example, 95% “identical” to a reference nucleotide sequence of the present invention, it is intended that the nucleotide sequence of the nucleic acid is identical to the reference sequence except that the nucleotide sequence may include up to five point mutations per each 100 nucleotides of the reference nucleotide sequence encoding the polypeptide. In other words, to obtain a nucleic acid having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. The query sequence may be an entire sequence referenced in Table I, IV, V, or VI, the ORF (open reading frame), or any fragment specified as described herein.

As a practical matter, whether any particular nucleic acid molecule or polypeptide is at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to a nucleotide sequence of the present invention can be determined conventionally using known computer programs. A preferred method for determining the best overall match between a query sequence (a sequence of the present invention) and a subject sequence, also referred to as a global sequence alignment, can be determined using the CLUSTALW computer program (Thompson, J. D., et al., Nucleic Acids Research, 2(22):4673-4680, (1994)), which is based on the algorithm of Higgins, D. G., et al., Computer Applications in the Biosciences (CABIOS), 8(2):189-191, (1992). In a sequence alignment the query and subject sequences are both DNA sequences. An RNA sequence can be compared by converting U's to T's. The result of said global sequence alignment is in percent identity. Preferred parameters used in a CLUSTALW alignment of DNA sequences to calculate percent identify are: Matrix=BLOSUM, k-tuple=1, Number of Top Diagonals=5, Gap Penalty=3, Gap Open Penalty 10, Gap Extension Penalty=0, Scoring Method=Percent, Window Size=5 or the length of the subject nucleotide sequence, whichever is shorter.

If the subject sequence is shorter than the query sequence because of 5′ or 3′ deletions, not because of internal deletions, a manual correction must be made to the results. This is because the CLUSTALW program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the CLUSTALW sequence alignment. This percentage is then subtracted from the percent identity, calculated by the above CLUSTALW program using the specified parameters, to arrive at a final percent identity score. This corrected score is what may be used for the purposes of the present invention. Only bases outside the 5′ and 3′ bases of the subject sequence, as displayed by the CLUSTALW alignment, which are not matched/aligned with the query sequence, are calculated for the purposes of manually adjusting the percent identity score.

For example, a 90 base subject sequence is aligned to a 100 base query sequence to determine percent identity. The deletions occur at the 5′ end of the subject sequence and therefore, the CLUSTALW alignment does not show a matched/alignment of the first 10 bases at 5′ end. The 10 unpaired bases represent 10% of the sequence (number of bases at the 5′ and 3′ ends not matched/total number of bases in the query sequence) so 10% is subtracted from the percent identity score calculated by the CLUSTALW program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by CLUSTALW is not manually corrected. Once again, only bases 5′ and 3′ of the subject sequence which are not matched/aligned with the query sequence are manually corrected for. No other manual corrections are required for the purposes of the present invention.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

As a practical matter, whether any particular polypeptide is at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to, for instance, an amino acid sequence referenced in Table I or Table VI (SEQ ID NO:6, 8, 10, 12, 14, 16, 18, 20, 22, 24, 26, 28, 30, 32, 34, 36, 38, 40, 42, 44, 46, 48, 50, and/or 604) can be determined conventionally using known computer programs. A preferred method for determining the best overall match between a query sequence (a sequence of the present invention) and a subject sequence, also referred to as a global sequence alignment, can be determined using the CLUSTALW computer program (Thompson, J. D., et al., Nucleic Acids Research, 2(22):4673-4680, (1994)), which is based on the algorithm of Higgins, D. G., et al., Computer Applications in the Biosciences (CABIOS), 8(2):189-191, (1992). In a sequence alignment the query and subject sequences are both DNA sequences. An RNA sequence can be compared by converting U's to T's. The result of said global sequence alignment is in percent identity. Preferred parameters used in a CLUSTALW amino acid alignment are: Matrix=BLOSUM, k-tuple=1, Number of Top Diagonals=5, Gap Penalty=3, Gap Open Penalty 10, Gap Extension Penalty=0, Scoring Method=Percent, Window Size=5 or the length of the subject nucleotide sequence, whichever is shorter.

If the subject sequence is shorter than the query sequence due to N- or C-terminal deletions, not because of internal deletions, a manual correction must be made to the results. This is because the CLUSTALW program does not account for N- and C-terminal truncations of the subject sequence when calculating global percent identity. For subject sequences truncated at the N- and C-terminal, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned,is determined by results of the CLUSTALW sequence alignment. This percentage is then subtracted from the percent identity, calculated by the above CLUSTALW program using the specified parameters, to arrive at a final percent identity score. This final percent identity score is what may be used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

For example, a 90 amino acid residue subject sequence is aligned with a 100 residue query sequence to determine percent identity. The deletion occurs at the N-terminus of the subject sequence and therefore, the CLUSTALW alignment does not show a matching/alignment of the first 10 residues at the N-terminus. The 10 unpaired residues represent 10% of the sequence (number of residues at the N- and C-terminl not matched/total number of residues in the query sequence) so 10% is subtracted from the percent identity score calculated by the CLUSTALW program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence, which are not matched/aligned with the query. In this case the percent identity calculated by CLUSTALW is not manually corrected. Once again, only residue positions outside the N- and C-terminal ends of the subject sequence, as displayed in the CLUSTALW alignment, which are not matched/aligned with the query sequence are manually corrected for. No other manual corrections are required for the purposes of the present invention.

The variants may contain alterations in the coding regions, non-coding regions, or both. Especially preferred are polynucleotide variants containing alterations which produce silent substitutions, additions, or deletions, but do not alter the properties or activities of the encoded polypeptide. Nucleotide variants produced by silent substitutions due to the degeneracy of the genetic code are preferred. Moreover, variants in which 5-10, 1-5, or 1-2 amino acids are substituted, deleted, or added in any combination are also preferred. Polynucleotide variants can be produced for a variety of reasons, e.g., to optimize codon expression for a particular host (change codons in the mRNA to those preferred by a bacterial host such as E. coli).

Naturally occurring variants are called “allelic variants,” and refer to one of several alternate forms of a gene occupying a given locus on a chromosome of an organism. (Genes II, Lewin, B., ed., John Wiley & Sons, New York (1985).) These allelic variants can vary at either the polynucleotide and/or polypeptide level and are included in the present invention. Alternatively, non-naturally occurring variants may be produced by mutagenesis techniques or by direct synthesis.

Using known methods of protein engineering and recombinant DNA technology, variants may be generated to improve or alter the characteristics of the polypeptides of the present invention. For instance, one or more amino acids can be deleted from the N-terminus or C-terminus of the protein without substantial loss of biological function. The authors of Ron et al., J. Biol. Chem. 268: 2984-2988 (1993), reported variant KGF proteins having heparin binding activity even after deleting 3, 8, or 27 amino-terminal amino acid residues. Similarly, Interferon gamma exhibited up to ten times higher activity after deleting 8-10 amino acid residues from the carboxy terminus of this protein (Dobeli et al., J. Biotechnology 7:199-216 (1988)).

Moreover, ample evidence demonstrates that variants often retain a biological activity similar to that of the naturally occurring protein. For example, Gayle and coworkers (J. Biol. Chem. 268:22105-22111 (1993)) conducted extensive mutational analysis of human cytokine IL-la. They used random mutagenesis to generate over 3,500 individual IL-1a mutants that averaged 2.5 amino acid changes per variant over the entire length of the molecule. Multiple mutations were examined at every possible amino acid position. The investigators found that “[m]ost of the molecule could be altered with little effect on either [binding or biological activity].” In fact, only 23 unique amino acid sequences, out of more than 3,500 nucleotide sequences examined, produced a protein that significantly differed in activity from wild-type.

Furthermore, even if deleting one or more amino acids from the N-terminus or C-terminus of a polypeptide results in modification or loss of one or more biological functions, other biological activities may still be retained. For example, the ability of a deletion variant to induce and/or to bind antibodies which recognize the protein will likely be retained when less than the majority of the residues of the protein are removed from the N-terminus or C-terminus. Whether a particular polypeptide lacking N- or C-terminal residues of a protein retains such immunogenic activities can readily be determined by routine methods described herein and otherwise known in the art.

Alternatively, such N-terminus or C-terminus deletions of a polypeptide of the present invention may, in fact, result in a significant increase in one or more of the biological activities of the polypeptide(s). For example, biological activity of many polypeptides are governed by the presence of regulatory domains at either one or both termini. Such regulatory domains effectively inhibit the biological activity of such polypeptides in lieu of an activation event (e.g., binding to a cognate ligand or receptor, phosphorylation, proteolytic processing, etc.). Thus, by eliminating the regulatory domain of a polypeptide, the polypeptide may effectively be rendered biologically active in the absence of an activation event.

Thus, the invention further includes polypeptide variants that show substantial biological activity. Such variants include deletions, insertions, inversions, repeats, and substitutions selected according to general rules known in the art so as have little effect on activity. For example, guidance concerning how to make phenotypically silent amino acid substitutions is provided in Bowie et al., Science 247:1306-1310 (1990), wherein the authors indicate that there are two main strategies for studying the tolerance of an amino acid sequence to change.

The first strategy exploits the tolerance of amino acid substitutions by natural selection during the process of evolution. By comparing amino acid sequences in different species, conserved amino acids can be identified. These conserved amino acids are likely important for protein function. In contrast, the amino acid positions where substitutions have been tolerated by natural selection indicates that these positions are not critical for protein function. Thus, positions tolerating amino acid substitution could be modified while still maintaining biological activity of the protein.

The second strategy uses genetic engineering to introduce amino acid changes at specific positions of a cloned gene to identify regions critical for protein function. For example, site directed mutagenesis or alanine-scanning mutagenesis (introduction of single alanine mutations at every residue in the molecule) can be used. (Cunningham and Wells, Science 244:1081-1085 (1989).) The resulting mutant molecules can then be tested for biological activity.

As the authors state, these two strategies have revealed that proteins are surprisingly tolerant of amino acid substitutions. The authors further indicate which amino acid changes are likely to be permissive at certain amino acid positions in the protein. For example, most buried (within the tertiary structure of the protein) amino acid residues require nonpolar side chains, whereas few features of surface side chains are generally conserved. Moreover, tolerated conservative amino acid substitutions involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

Besides conservative amino acid substitution, variants of the present invention include, but are not limited to, the following: (i) substitutions with one or more of the non-conserved amino acid residues, where the substituted amino acid residues may or may not be one encoded by the genetic code, or (ii) substitution with one or more of amino acid residues having a substituent group, or (iii) fusion of the mature polypeptide with another compound, such as a compound to increase the stability and/or solubility of the polypeptide (for example, polyethylene glycol), or (iv) fusion of the polypeptide with additional amino acids, such as, for example, an IgG Fc fusion region peptide, or leader or secretory sequence, or a sequence facilitating purification. Such variant polypeptides are deemed to be within the scope of those skilled in the art from the teachings herein.

For example, polypeptide variants containing amino acid substitutions of charged amino acids with other charged or neutral amino acids may produce proteins with improved characteristics, such as less aggregation. Aggregation of pharmaceutical formulations both reduces activity and increases clearance due to the aggregate's immunogenic activity. (Pinckard et al., Clin. Exp. Immunol. 2:331-340 (1967); Robbins et al., Diabetes 36: 838-845 (1987); Cleland et al., Crit. Rev. Therapeutic Drug Carrier Systems 10:307-377 (1993).)

Moreover, the invention further includes polypeptide variants created through the application of molecular evolution (“DNA Shuffling”) methodology to the polynucleotide disclosed as SEQ ID NO:5, 7, 9, 11, 13, 15, 17, 19, 21, 23, 25, 27, 29, 31, 33, 35, 37, 39, 41, 43, 45, 47, 49, and/or 603, and/or the cDNA encoding the polypeptide disclosed as SEQ ID NO:6, 8, 10, 12, 14, 16, 18, 20, 22, 24, 26, 28, 30, 32, 34, 36, 38, 40, 42, 44, 46, 48, 50, and/or 604. Such DNA Shuffling technology is known in the art and more particularly described elsewhere herein (e.g., WPC, Stemmer, PNAS, 91:10747, (1994)), and in the Examples provided herein).

A further embodiment of the invention relates to a polypeptide which comprises the amino acid sequence of the present invention having an amino acid sequence which contains at least one amino acid substitution, but not more than 50 amino acid substitutions, even more preferably, not more than 40 amino acid substitutions, still more preferably, not more than 30 amino acid substitutions, and still even more preferably, not more than 20 amino acid substitutions. Of course, in order of ever-increasing preference, it is highly preferable for a peptide or polypeptide to have an amino acid sequence which comprises the amino acid sequence of the present invention, which contains at least one, but not more than 10, 9, 8, 7, 6, 5, 4, 3, 2 or 1 amino acid substitutions. In specific embodiments, the number of additions, substitutions, and/or deletions in the amino acid sequence of the present invention or fragments thereof (e.g., the mature form and/or other fragments described herein), is 1-5, 5-10, 5-25, 5-50, 10-50 or 50-150, conservative amino acid substitutions are preferable.

### Polynucleotide and Polypeptide Fragments

The present invention is directed to polynucleotide fragments of the polynucleotides of the invention, in addition to polypeptides encoded therein by said polynucleotides and/or fragments.

In the present invention, a “polynucleotide fragment” refers to a short polynucleotide having a nucleic acid sequence which: is a portion of that shown in SEQ ID NO:5, 7, 9, 11, 13, 15, 17, 19, 21, 23, 25, 27, 29, 31, 33, 35, 37, 39, 41, 43, 45, 47, 49, and/or 603 or the complementary strand thereto, or is a portion of a polynucleotide sequence encoding the polypeptide of SEQ ID NO:6, 8, 10, 12, 14, 16, 18, 20, 22, 24, 26, 28, 30, 32, 34, 36, 38, 40, 42, 44, 46, 48, 50, and/or 604. The nucleotide fragments of the invention are preferably at least about 15 nt, and more preferably at least about 20 nt, still more preferably at least about 30 nt, and even more preferably, at least about 40 nt, at least about 50 nt, at least about 75 nt, or at least about 150 nt in length. A fragment “at least 20 nt in length,” for example, is intended to include 20 or more contiguous bases from the CDNA sequence shown in SEQ ID NO:5, 7, 9, 11, 13, 15, 17, 19, 21, 23, 25, 27, 29, 31, 33, 35, 37, 39, 41, 43, 45, 47, 49, and/or 603. In this context “about” includes the particularly recited value, a value larger or smaller by several (5, 4, 3, 2, or 1) nucleotides, at either terminus, or at both termini. These nucleotide fragments have uses that include, but are not limited to, as diagnostic probes and primers as discussed herein. Of course, larger fragments (e.g., 50, 150, 500, 600, 2000 nucleotides) are preferred.

Moreover, representative examples of polynucleotide fragments of the invention, include, for example, fragments comprising, or alternatively consisting of, a sequence from about nucleotide number 1-50, 51-100, 101-150, 151-200, 201-250, 251-300, 301-350, 351-400, 401-450, 451-500, 501-550, 551-600, 651-700, 701-750, 751-800, 800-850, 851-900, 901-950, 951-1000, 1001-1050, 1051-1100, 1101-1150, 1151-1200, 1201-1250, 1251-1300, 1301-1350, 1351-1400, 1401-1450, 1451-1500, 1501-1550, 1551-1600, 1601-1650, 1651-1700, 1701-1750, 1751-1800, 1801-1850, 1851-1900, 1901-1950, 1951-2000, or 2001 to the end of SEQ ID NO:5, 7, 9, 11, 13, 15, 17, 19, 21, 23, 25, 27, 29, 31, 33, 35, 37, 39, 41, 43, 45, 47, 49, and/or 603, or the complementary strand thereto. In this context “about” includes the particularly recited ranges, and ranges larger or smaller by several (5, 4, 3, 2, or 1) nucleotides, at either terminus or at both termini. Preferably, these fragments encode a polypeptide which has biological activity. More preferably, these polynucleotides can be used as probes or primers as discussed herein. Also encompassed by the present invention are polynucleotides which hybridize to these nucleic acid molecules under stringent hybridization conditions or lower stringency conditions, as are the polypeptides encoded by these polynucleotides.

In the present invention, a “polypeptide fragment” refers to an amino acid sequence which is a portion of that contained in SEQ ID NO:6, 8, 10, 12, 14, 16, 18, 20, 22, 24, 26, 28, 30, 32, 34, 36, 38, 40, 42, 44, 46, 48, 50, and/or 604. Protein (polypeptide) fragments may be “free-standing,” or comprised within a larger polypeptide of which the fragment forms a part or region, most preferably as a single continuous region. Representative examples of polypeptide fragments of the invention, include, for example, fragments comprising, or alternatively consisting of, from about amino acid number 1-20, 21-40, 41-60, 61-80, 81-100, 102-120, 121-140, 141-160, or 161 to the end of the coding region. Moreover, polypeptide fragments can be about 20, 30, 40, 50, 60, 70, 80, 90, 100, 110, 120, 130, 140, or 150 amino acids in length. In this context “about” includes the particularly recited ranges or values, and ranges or values larger or smaller by several (5, 4, 3, 2, or 1) amino acids, at either extreme or at both extremes. Polynucleotides encoding these polypeptides are also encompassed by the invention.

Preferred polypeptide fragments include the full-length protein. Further preferred polypeptide fragments include the full-length protein having a continuous series of deleted residues from the amino or the carboxy terminus, or both. For example, any number of amino acids, ranging from 1-60, can be deleted from the amino terminus of the full-length polypeptide. Similarly, any number of amino acids, ranging from 1-30, can be deleted from the carboxy terminus of the full-length protein. Furthermore, any combination of the above amino and carboxy terminus deletions are preferred. Similarly, polynucleotides encoding these polypeptide fragments are also preferred.

Also preferred are polypeptide and polynucleotide fragments characterized by structural or functional domains, such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions. Polypeptide fragments of SEQ ID NO:6, 8, 10, 12, 14, 16, 18, 20, 22, 24, 26, 28, 30, 32, 34, 36, 38, 40, 42, 44, 46, 48, 50, and/or 604 falling within conserved domains are specifically contemplated by the present invention. Moreover, polynucleotides encoding these domains are also contemplated.

Other preferred polypeptide fragments are biologically active fragments. Biologically active fragments are those exhibiting activity similar, but not necessarily identical, to an activity of the polypeptide of the present invention. The biological activity of the fragments may include an improved desired activity, or a decreased undesirable activity. Polynucleotides encoding these polypeptide fragments are also encompassed by the invention.

In a preferred embodiment, the functional activity displayed by a polypeptide encoded by a polynucleotide fragment of the invention may be one or more biological activities typically associated with the full-length polypeptide of the invention. Illustrative of these biological activities includes the fragments ability to bind to at least one of the same antibodies which bind to the full-length protein, the fragments ability to interact with at lease one of the same proteins which bind to the full-length, the fragments ability to elicit at least one of the same immune responses as the full-length protein (i.e., to cause the immune system to create antibodies specific to the same epitope, etc.), the fragments ability to bind to at least one of the same polynucleotides as the full-length protein, the fragments ability to bind to a receptor of the full-length protein, the fragments ability to bind to a ligand of the full-length protein, and the fragments ability to multimerize with the full-length protein. However, the skilled artisan would appreciate that some fragments may have biological activities which are desirable and directly inapposite to the biological activity of the full-length protein. The functional activity of polypeptides of the invention, including fragments, variants, derivatives, and analogs thereof can be determined by numerous methods available to the skilled artisan, some of which are described elsewhere herein.

The present invention encompasses polypeptides comprising, or alternatively consisting of, an epitope of the polypeptide having an amino acid sequence of SEQ ID NO:6, 8, 10, 12, 14, 16, 18, 20, 22, 24, 26, 28, 30, 32, 34, 36, 38, 40, 42, 44, 46, 48, 50, and/or 604, or encoded by a polynucleotide that hybridizes to the complement of the sequence of SEQ ID NO:5, 7, 9, 11, 13, 15, 17, 19, 21, 23, 25, 27, 29, 31, 33, 35, 37, 39, 41, 43, 45, 47, 49, and/or 603 under stringent hybridization conditions or lower stringency hybridization conditions as defined supra. The present invention further encompasses polynucleotide sequences encoding an epitope of a polypeptide sequence of the invention (such as, for example, the sequence disclosed in SEQ ID NO:1), polynucleotide sequences of the complementary strand of a polynucleotide sequence encoding an epitope of the invention, and polynucleotide sequences which hybridize to the complementary strand under stringent hybridization conditions or lower stringency hybridization conditions defined supra.

The term “epitopes,” as used herein, refers to portions of a polypeptide having antigenic or immunogenic activity in an animal, preferably a mammal, and most preferably in a human. In a preferred embodiment, the present invention encompasses a polypeptide comprising an epitope, as well as the polynucleotide encoding this polypeptide. An “immunogenic epitope,” as used herein, is defined as a portion of a protein that elicits an antibody response in an animal, as determined by any method known in the art, for example, by the methods for generating antibodies described infra. (See, for example, Geysen et al., Proc. Natl. Acad. Sci. USA 81:3998-4002 (1983)). The term “antigenic epitope,” as used herein, is defined as a portion of a protein to which an antibody can immunospecifically bind its antigen as determined by any method well known in the art, for example, by the immunoassays described herein. Immunospecific binding excludes non-specific binding but does not necessarily exclude cross-reactivity with other antigens. Antigenic epitopes need not necessarily be immunogenic.

Fragments which function as epitopes may be produced by any conventional means. (See, e.g., Houghten, Proc. Natl. Acad. Sci. USA 82:5131-5135 (1985), further described in U.S. Pat. No. 4,631,211).

In the present invention, antigenic epitopes preferably contain a sequence of at least 4, at least 5, at least 6, at least 7, more preferably at least 8, at least 9, at least 10, at least 11, at least 12, at least 13, at least 14, at least 15, at least 20, at least 25, at least 30, at least 40, at least 50, and, most preferably, between about 15 to about 30 amino acids. Preferred polypeptides comprising immunogenic or antigenic epitopes are at least 10, 15, 20, 25, 30, 35, 40, 45, 50, 55, 60, 65, 70, 75, 80, 85, 90, 95, or 100 amino acid residues in length, or longer. Additional non-exclusive preferred antigenic epitopes include the antigenic epitopes disclosed herein, as well as portions thereof. Antigenic epitopes are useful, for example, to raise antibodies, including monoclonal antibodies, that specifically bind the epitope. Preferred antigenic epitopes include the antigenic epitopes disclosed herein, as well as any combination of two, three, four, five or more of these antigenic epitopes. Antigenic epitopes can be used as the target molecules in immunoassays. (See, for instance, Wilson et al., Cell 37:767-778 (1984); Sutcliffe et al., Science 219:660-666 (1983)).

Similarly, immunogenic epitopes can be used, for example, to induce antibodies according to methods well known in the art. (See, for instance, Sutcliffe et al., supra; Wilson et al., supra; Chow et al., Proc. Natl. Acad. Sci. USA 82:910-914; and Bittle et al., J. Gen. Virol. 66:2347-2354 (1985). Preferred immunogenic epitopes include the immunogenic epitopes disclosed herein, as well as any combination of two, three, four, five or more of these immunogenic epitopes. The polypeptides comprising one or more immunogenic epitopes may be presented for eliciting an antibody response together with a carrier protein, such as an albumin, to an animal system (such as rabbit or mouse), or, if the polypeptide is of sufficient length (at least about 25 amino acids), the polypeptide may be presented without a carrier. However, immunogenic epitopes comprising as few as 8 to 10 amino acids have been shown to be sufficient to raise antibodies capable of binding to, at the very least, linear epitopes in a denatured polypeptide (e.g., in Western blotting).

Epitope-bearing polypeptides of the present invention may be used to induce antibodies according to methods well known in the art including, but not limited to, in vivo immunization, in vitro immunization, and phage display methods. See, e.g., Sutcliffe et al., supra; Wilson et al., supra, and Bittle et al., J. Gen. Virol., 66:2347-2354 (1985). If in vivo immunization is used, animals may be immunized with free peptide; however, anti-peptide antibody titer may be boosted by coupling the peptide to a macromolecular carrier, such as keyhole limpet hemacyanin (KLH) or tetanus toxoid. For instance, peptides containing cysteine residues may be coupled to a carrier using a linker such as maleimidobenzoyl-N-hydroxysuccinimide ester (MBS), while other peptides may be coupled to carriers using a more general linking agent such as glutaraldehyde. Animals such as rabbits, rats and mice are immunized with either free or carrier-coupled peptides, for instance, by intraperitoneal and/or intradermal injection of emulsions containing about 100 μg of peptide or carrier protein and Freund's adjuvant or any other adjuvant known for stimulating an immune response. Several booster injections may be needed, for instance, at intervals of about two weeks, to provide a useful titer of anti-peptide antibody which can be detected, for example, by ELISA assay using free peptide adsorbed to a solid surface. The titer of anti-peptide antibodies in serum from an immunized animal may be increased by selection of anti-peptide antibodies, for instance, by adsorption to the peptide on a solid support and elution of the selected antibodies according to methods well known in the art.

As one of skill in the art will appreciate, and as discussed above, the polypeptides of the present invention comprising an immunogenic or antigenic epitope can be fused to other polypeptide sequences. For example, the polypeptides of the present invention may be fused with the constant domain of immunoglobulins (IgA, IgE, IgG, IgM), or portions thereof (CH1, CH2, CH3, or any combination thereof and portions thereof) resulting in chimeric polypeptides. Such fusion proteins may facilitate purification and may increase half-life in vivo. This has been shown for chimeric proteins consisting of the first two domains of the human CD4-polypeptide and various domains of the constant regions of the heavy or light chains of mammalian immunoglobulins. See, e.g., EP 394,827; Traunecker et al., Nature, 331:84-86 (1988). Enhanced delivery of an antigen across the epithelial barrier to the immune system has been demonstrated for antigens (e.g., insulin) conjugated to an FcRn binding partner such as IgG or Fc fragments (see, e.g., PCT Publications WO 96/22024 and WO 99/04813). IgG Fusion proteins that have a disulfide-linked dimeric structure due to the IgG portion disulfide bonds have also been found to be more efficient in binding and neutralizing other molecules than monomeric polypeptides or fragments thereof alone. See, e.g., Fountoulakis et al., J. Biochem., 270:3958-3964 (1995). Nucleic acids encoding the above epitopes can also be recombined with a gene of interest as an epitope tag (e.g., the hemagglutinin (“HA”) tag or flag tag) to aid in detection and purification of the expressed polypeptide. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with the recombinant vaccinia virus are loaded onto Ni2+ nitriloacetic acid-agarose column and histidine-tagged proteins can be selectively eluted with imidazole-containing buffers.

Additional fusion proteins of the invention may be generated through the techniques of gene-shuffling, motif-shuffling, exon-shuffling, and/or codon-shuffling (collectively referred to as “DNA shuffling”). DNA shuffling may be employed to modulate the activities of polypeptides of the invention, such methods can be used to generate polypeptides with altered activity, as well as agonists and antagonists of the polypeptides. See, generally, U.S. Pat. Nos. 5,605,793; 5,811,238; 5,830,721; 5,834,252; and 5,837,458, and Patten et al., Curr. Opinion Biotechnol. 8:724-33 (1997); Harayama, Trends Biotechnol. 16(2):76-82 (1998); Hansson, et al., J. Mol. Biol. 287:265-76 (1999); and Lorenzo and Blasco, Biotechniques 24(2):308-13 (1998) (each of these patents and publications are hereby incorporated by reference in its entirety). In one embodiment, alteration of polynucleotides corresponding to SEQ ID NO:5, 7, 9, 11, 13, 15, 17, 19, 21, 23, 25, 27, 29, 31, 33, 35, 37, 39, 41, 43, 45, 47, 49, and/or 603 and the polypeptides encoded by these polynucleotides may be achieved by DNA shuffling. DNA shuffling involves the assembly of two or more DNA segments by homologous or site-specific recombination to generate variation in the polynucleotide sequence. In another embodiment, polynucleotides of the invention, or the encoded polypeptides, may be altered by being subjected to random mutagenesis by error-prone PCR, random nucleotide insertion or other methods prior to recombination. In another embodiment, one or more components, motifs, sections, parts, domains, fragments, etc., of a polynucleotide encoding a polypeptide of the invention may be recombined with one or more components, motifs, sections, parts, domains, fragments, etc. of one or more heterologous molecules.

### Antibodies

Further polypeptides of the invention relate to antibodies and T-cell antigen receptors (TCR) which immunospecifically bind a polypeptide, polypeptide fragment, or variant of SEQ ID NO:6, 8, 10, 12, 14, 16, 18, 20, 22, 24, 26, 28, 30, 32, 34, 36, 38, 40, 42, 44, 46, 48, 50, and/or 604, and/or an epitope, of the present invention (as determined by immunoassays well known in the art for assaying specific antibody-antigen binding). Antibodies of the invention include, but are not limited to, polyclonal, monoclonal, monovalent, bispecific, heteroconjugate, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule. Moreover, the term “antibody” (Ab) or “monoclonal antibody” (Mab) is meant to include intact molecules, as well as, antibody fragments (such as, for example, Fab and F(ab′)2 fragments) which are capable of specifically binding to protein. Fab and F(ab′)2 fragments lack the Fc fragment of intact antibody, clear more rapidly from the circulation of the animal or plant, and may have less non-specific tissue binding than an intact antibody (Wahl et al., J. Nucl. Med. 24:316-325 (1983)). Thus, these fragments are preferred, as well as the products of a FAB or other immunoglobulin expression library. Moreover, antibodies of the present invention include chimeric, single chain, and humanized antibodies.

Most preferably the antibodies are human antigen-binding antibody fragments of the present invention and include, but are not limited to, Fab, Fab′ and F(ab′)2, Fd, single-chain Fvs (scFv), single-chain antibodies, disulfide-linked Fvs (sdFv) and fragments comprising either a VL or VH domain. Antigen-binding antibody fragments, including single-chain antibodies, may comprise the variable region(s) alone or in combination with the entirety or a portion of the following: hinge region, CH1, CH2, and CH3 domains. Also included in the invention are antigen-binding fragments also comprising any combination of variable region(s) with a hinge region, CH1, CH2, and CH3 domains. The antibodies of the invention may be from any animal origin including birds and mammals. Preferably, the antibodies are human, murine (e.g., mouse and rat), donkey, ship rabbit, goat, guinea pig, camel, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described infra and, for example in, U.S. Pat. No. 5,939,598 by Kucherlapati et al.

The antibodies of the present invention may be monospecific, bispecific, trispecific or of greater multispecificity. Multispecific antibodies may be specific for different epitopes of a polypeptide of the present invention or may be specific for both a polypeptide of the present invention as well as for a heterologous epitope, such as a heterologous polypeptide or solid support material. See, e.g., PCT publications WO 93/17715; WO 92/08802; WO 91/00360; WO 92/05793; Tutt, et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992).

Antibodies of the present invention may be described or specified in terms of the epitope(s) or portion(s) of a polypeptide of the present invention which they recognize or specifically bind. The epitope(s) or polypeptide portion(s) may be specified as described herein, e.g., by N-terminal and C-terminal positions, by size in contiguous amino acid residues, or listed in the Tables and Figures. Antibodies which specifically bind any epitope or polypeptide of the present invention may also be excluded. Therefore, the present invention includes antibodies that specifically bind polypeptides of the present invention, and allows for the exclusion of the same.

Antibodies of the present invention may also be described or specified in terms of their cross-reactivity. Antibodies that do not bind any other analog, ortholog, or homologue of a polypeptide of the present invention are included. Antibodies that bind polypeptides with at least 95%, at least 90%, at least 85%, at least 80%, at least 75%, at least 70%, at least 65%, at least 60%, at least 55%, and at least 50% identity (as calculated using methods known in the art and described herein) to a polypeptide of the present invention are also included in the present invention. In specific embodiments, antibodies of the present invention cross-react with murine, rat and/or rabbit homologues of human proteins and the corresponding epitopes thereof. Antibodies that do not bind polypeptides with less than 95%, less than 90%, less than 85%, less than 80%, less than 75%, less than 70%, less than 65%, less than 60%, less than 55%, and less than 50% identity (as calculated using methods known in the art and described herein) to a polypeptide of the present invention are also included in the present invention. In a specific embodiment, the above-described cross-reactivity is with respect to any single specific antigenic or immunogenic polypeptide, or combination(s) of 2, 3, 4, 5, or more of the specific antigenic and/or immunogenic polypeptides disclosed herein. Further included in the present invention are antibodies which bind polypeptides encoded by polynucleotides which hybridize to a polynucleotide of the present invention under stringent hybridization conditions (as described herein). Antibodies of the present invention may also be described or specified in terms of their binding affinity to a polypeptide of the invention. Preferred binding affinities include those with a dissociation constant or Kd less than 5×10-2 M, 10-2 M, 5×10-3 M, 10-3 M, 5×10-4 M, 10-4 M, 5×10-5 M, 10-5 M, 5×10-6 M, 10-6M, 5×10-7 M, 107 M, 5×10-8 M, 10-8 M, 5×10-9 M, 10-9 M, 5×10-10 M, 10-10 M, 5×10-11 M, 10-11 M, 5×10-12 M, 10-12 M, 5×10-13 M, 10-13 M, 5×10-14 M, 10-14 M, 5×10-15 M, or 10-15 M.

The invention also provides antibodies that competitively inhibit binding of an antibody to an epitope of the invention as determined by any method known in the art for determining competitive binding, for example, the immunoassays described herein. In preferred embodiments, the antibody competitively inhibits binding to the epitope by at least 95%, at least 90%, at least 85%, at least 80%, at least 75%, at least 70%, at least 60%, or at least 50%.

Antibodies of the present invention may act as agonists or antagonists of the polypeptides of the present invention. For example, the present invention includes antibodies which disrupt the receptor/ligand interactions with the polypeptides of the invention either partially or fully. Preferably, antibodies of the present invention bind an antigenic epitope disclosed herein, or a portion thereof. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or its substrate by immunoprecipitation followed by western blot analysis (for example, as described supra). In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex, and, preferably, do not specifically recognize the unbound receptor or the unbound ligand. Likewise, included in the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides of the invention disclosed herein. The above antibody agonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. 111(Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996) (which are all incorporated by reference herein in their entireties).

Antibodies of the present invention may be used, for example, but not limited to, to purify, detect, and target the polypeptides of the present invention, including both in vitro and in vivo diagnostic and therapeutic methods. For example, the antibodies have use in immunoassays for qualitatively and quantitatively measuring levels of the polypeptides of the present invention in biological samples. See, e.g., Harlow et al., Antibodies: A Laboratory Manual, (Cold Spring Harbor Laboratory Press, 2nd ed. 1988) (incorporated by reference herein in its entirety).

As discussed in more detail below, the antibodies of the present invention may be used either alone or in combination with other compositions. The antibodies may further be recombinantly fused to a heterologous polypeptide at the N- or C-terminus or chemically conjugated (including covalently and non-covalently conjugations) to polypeptides or other compositions. For example, antibodies of the present invention may be recombinantly fused or conjugated to molecules useful as labels in detection assays and effector molecules such as heterologous polypeptides, drugs, radionucleotides, or toxins. See, e.g., PCT publications WO 92/08495; WO 91/14438; WO 89/12624; U.S. Pat. No. 5,314,995; and EP 396,387.

The antibodies of the invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

The antibodies of the present invention may be generated by any suitable method known in the art.

The antibodies of the present invention may comprise polyclonal antibodies. Methods of preparing polyclonal antibodies are known to the skilled artisan (Harlow, et al., Antibodies: A Laboratory Manual, (Cold spring Harbor Laboratory Press, 2^{nd} ed. (1988); and Current Protocols, Chapter 2; which are hereby incorporated herein by reference in its entirety). In a preferred method, a preparation of the polymorphic protein is prepared and purified to render it substantially free of natural contaminants. Such a preparation is then introduced into an animal in order to produce polyclonal antisera of greater specific activity. For example, a polypeptide of the invention can be administered to various host animals including, but not limited to, rabbits, mice, rats, etc. to induce the production of sera containing polyclonal antibodies specific for the antigen. The administration of the polypeptides of the present invention may entail one or more injections of an immunizing agent and, if desired, an adjuvant. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art. For the purposes of the invention, “immunizing agent” may be defined as a polypeptide of the invention, including fragments, variants, and/or derivatives thereof, in addition to fusions with heterologous polypeptides and other forms of the polypeptides described herein.

Typically, the immunizing agent and/or adjuvant will be injected in the mammal by multiple subcutaneous or intraperitoneal injections, though they may also be given intramuscularly, and/or through IV). The immunizing agent may include polypeptides of the present invention or a fusion protein or variants thereof. Depending upon the nature of the polypeptides (i.e., percent hydrophobicity, percent hydrophilicity, stability, net charge, isoelectric point etc.), it may be useful to conjugate the immunizing agent to a protein known to be immunogenic in the mammal being immunized. Such conjugation includes either chemical conjugation by derivitizing active chemical functional groups to both the polypeptide of the present invention and the immunogenic protein such that a covalent bond is formed, or through fusion-protein based methodology, or other methods known to the skilled artisan. Examples of such immunogenic proteins include, but are not limited to keyhole limpet hemocyanin, serum albumin, bovine thyroglobulin, and soybean trypsin inhibitor. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum. Additional examples of adjuvants which may be employed includes the MPL-TDM adjuvant (monophosphoryl lipid A, synthetic trehalose dicorynomycolate). The immunization protocol may be selected by one skilled in the art without undue experimentation.

The antibodies of the present invention may comprise monoclonal antibodies. Monoclonal antibodies may be prepared using hybridoma methods, such as those described by Kohler and Milstein, Nature, 256:495 (1975) and U.S. Pat. No. 4,376,110, by Harlow, et al., Antibodies: A Laboratory Manual, (Cold spring Harbor Laboratory Press, 2^{nd} ed. (1988), by Hammerling, et al., Monoclonal Antibodies and T-Cell Hybridomas (Elsevier, N.Y., pp. 563-681 (1981); Köhler et al., Eur. J. Immunol. 6:511 (1976); Köhler et al., Eur. J. Immunol. 6:292 (1976), or other methods known to the artisan. Other examples of methods which may be employed for producing monoclonal antibodies includes, but are not limited to, the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In a hybridoma method, a mouse, a humanized mouse, a mouse with a human immune system, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include polypeptides of the present invention or a fusion protein thereof. Preferably, the immunizing agent consists of an polymorphic polypeptide or, more preferably, with a polymorphic polypeptide-expressing cell. Such cells may be cultured in any suitable tissue culture medium; however, it is preferable to culture cells in Earle's modified Eagle's medium supplemented with 10% fetal bovine serum (inactivated at about 56 degrees C), and supplemented with about 10 g/l of nonessential amino acids, about 1,000 U/ml of penicillin, and about 100 ug/ml of streptomycin. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986), pp. 59-103). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. More preferred are the parent myeloma cell line (SP2O) as provided by the ATCC. As inferred throughout the specification, human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the polypeptides of the present invention. Preferably, the binding specificity of monoclonal antibodies produced by the hybridoma cells is determined by immunoprecipitation or by an in vitro binding assay, such as radioimmunoassay (RIA) or enzyme-linked immunoabsorbant assay (ELISA). Such techniques are known in the art and within the skill of the artisan. The binding affinity of the monoclonal antibody can, for example, be determined by the Scatchard analysis of Munson and Pollart, Anal. Biochem., 107:220 (1980).

After the desired hybridoma cells are identified, the clones may be subcloned by limiting dilution procedures and grown by standard methods (Goding, supra, and/or according to Wands et al. (Gastroenterology 80:225-232 (1981)). Suitable culture media for this purpose include, for example, Dulbecco's Modified Eagle's Medium and RPMI-1640. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-sepharose, hydroxyapatite chromatography, gel exclusion chromatography, gel electrophoresis, dialysis, or affinity chromatography.

The skilled artisan would acknowledge that a variety of methods exist in the art for the production of monoclonal antibodies and thus, the invention is not limited to their sole production in hydridomas. For example, the monoclonal antibodies may be made by recombinant DNA methods, such as those described in U.S. Pat. No. 4,816,567. In this context, the term “monoclonal antibody” refers to an antibody derived from a single eukaryotic, phage, or prokaryotic clone. The DNA encoding the monoclonal antibodies of the invention can be readily isolated and sequenced using conventional procedures (e.g., by using oligonucleotide probes that are capable of binding specifically to genes encoding the heavy and light chains of murine antibodies, or such chains from human, humanized, or other sources). The hydridoma cells of the invention serve as a preferred source of such DNA. Once isolated, the DNA may be placed into expression vectors, which are then transformed into host cells such as Simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567; Morrison et al, supra) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art. Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988) Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art and are discussed in detail in the Examples described herein. In a non-limiting example, mice can be immunized with a polypeptide of the invention or a cell expressing such peptide. Once an immune response is detected, e.g., antibodies specific for the antigen are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, the present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with an antigen of the invention with myeloma cells and then screening the hybridomas resulting from the fusion for hybridoma clones that secrete an antibody able to bind a polypeptide of the invention.

Antibody fragments which recognize specific epitopes may be generated by known techniques. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain.

For example, the antibodies of the present invention can also be generated using various phage display methods known in the art. In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties). Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988).

For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science 229:1202 (1985); Oi et al., BioTechniques 4:214 (1986); Gillies et al., (1989) J. Immunol. Methods 125:191-202; Cabilly et al., Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985); U.S. Pat. Nos. 5,807,715; 4,816,567; and 4,816397, which are incorporated herein by reference in their entirety. Humanized antibodies are antibody molecules from non-human species antibody that binds the desired antigen having one or more complementarity determining regions (CDRs) from the non-human species and a framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332). Generally, a humanized antibody has one or more amino acid residues introduced into it from a source that is non-human. These non-human amino acid residues are often referred to as “import” residues, which are typically taken from an “import” variable domain. Humanization can be essentially performed following the methods of Winter and co-workers (Jones et al., Nature, 321:522-525 (1986); Reichmann et al., Nature, 332:323-327 (1988); Verhoeyen et al., Science, 239:1534-1536 (1988), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possible some FR residues are substituted from analogous sites in rodent antibodies.

In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature 332:323-329 (1988)1 and Presta, Curr. Op. Struct. Biol., 2:593-596 (1992).

Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety. The techniques of cole et al., and Boerder et al., are also available for the preparation of human monoclonal antibodies (cole et al., Monoclonal Antibodies and Cancer Therapy, Alan R. Riss, (1985); and Boerner et al., J. Immunol., 147(1):86-95, (1991)).

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, Int. Rev. Immunol. 13:65-93 (1995). For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference herein in their entirety. In addition, companies such as Abgenix, Inc. (Freemont, Calif.), Genpharm (San Jose, Calif.), and Medarex, Inc. (Princeton, N.J.) can be engaged to provide human antibodies directed against a selected antigen using technology similar to that described above.

Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and creation of an antibody repertoire. This approach is described, for example, in U.S. Pat. Nos. 5,545,807; 5,545,806; 5,569,825; 5,625,126; 5,633,425; 5,661,106, and in the following scientific publications: Marks et al., Biotechnol., 10:779-783 (1992); Lonberg et al., Nature 368:856-859 (1994); Fishwild et al., Nature Biotechnol., 14:845-51 (1996); Neuberger, Nature Biotechnol., 14:826 (1996); Lonberg and Huszer, Intern. Rev. Immunol., 13:65-93 (1995).

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/technology 12:899-903 (1988)).

Further, antibodies to the polypeptides of the invention can, in turn, be utilized to generate anti-idiotype antibodies that “mimic” polypeptides of the invention using techniques well known to those skilled in the art. (See, e.g., Greenspan & Bona, FASEB J. 7(5):437-444; (1989) and Nissinoff, J. Immunol. 147(8):2429-2438 (1991)). For example, antibodies which bind to and competitively inhibit polypeptide multimerization and/or binding of a polypeptide of the invention to a ligand can be used to generate anti-idiotypes that “mimic” the polypeptide multimerization and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti-idiotypic antibodies can be used to bind a polypeptide of the invention and/or to bind its ligands/receptors, and thereby block its biological activity.

Such anti-idiotypic antibodies capable of binding to the polymorphic polypeptide can be produced in a two-step procedure. Such a method makes use of the fact that antibodies are themselves antigens, and therefore, it is possible to obtain an antibody that binds to a second antibody. In accordance with this method, protein specific antibodies are used to immunize an animal, preferably a mouse. The splenocytes of such an animal are then used to produce hybridoma cells, and the hybridoma cells are screened to identify clones that produce an antibody whose ability to bind to the protein-specific antibody can be blocked by the polypeptide. Such antibodies comprise anti-idiotypic antibodies to the protein-specific antibody and can be used to immunize an animal to induce formation of further protein-specific antibodies.

The antibodies of the present invention may be bispecific antibodies. Bispecific antibodies are monoclonal, Preferably human or humanized, antibodies that have binding specificities for at least two different antigens. In the present invention, one of the binding specificities may be directed towards a polypeptide of the present invention, the other may be for any other antigen, and preferably for a cell-surface protein, receptor, receptor subunit, tissue-specific antigen, virally derived protein, virally encoded envelope protein, bacterially derived protein, or bacterial surface protein, etc.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography, steps. Similar procedures are disclosed in WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transformed into a suitable host organism. For further details of generating bispecific antibodies see, for example Suresh et al., Meth. In Enzym., 121:210 (1986).

Heteroconjugate antibodies are also contemplated by the present invention. Heteroconjugate antibodies are composed of two covalently joined antibodies. Such antibodies have, for example, been proposed to target immune system cells to unwanted cells (U.S. Pat. No. 4,676,980), and for the treatment of HIV infection (WO 91/00360; WO 92/20373; and EP03089). It is contemplated that the antibodies may be prepared in vitro using known methods in synthetic protein chemistry, including those involving crosslinking agents. For example, immunotoxins may be constructed using a disulfide exchange reaction or by forming a thioester bond. Examples of suitable reagents for this purpose include iminothiolate and methyl-4-mercaptobutyrimidate and those disclosed, for example, in U.S. Pat. No. 4,676,980.

### Polynucleotides Encoding Antibodies

The invention further provides polynucleotides comprising a nucleotide sequence encoding an antibody of the invention and fragments thereof. The invention also encompasses polynucleotides that hybridize under stringent or lower stringency hybridization conditions, e.g., as defined supra, to polynucleotides that encode an antibody, preferably, that specifically binds to a polypeptide of the invention, preferably, an antibody that binds to a polypeptide having the amino acid sequence of SEQ ID NO:6, 8, 10, 12, 14, 16, 18, 20, 22, 24, 26, 28, 30, 32, 34, 36, 38, 40, 42, 44, 46, 48, 50, and/or 604.

The polynucleotides may be obtained, and the nucleotide sequence of the polynucleotides determined, by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A+RNA, isolated from, any tissue or cells expressing the antibody, such as hybridoma cells selected to express an antibody of the invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence and corresponding amino acid sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties ), to generate antibodies having a different amino acid sequence, for example to create amino acid substitutions, deletions, and/or insertions.

In a specific embodiment, the amino acid sequence of the heavy and/or light chain variable domains may be inspected to identify the sequences of the complementarity determining regions (CDRs) by methods that are well know in the art, e.g., by comparison to known amino acid sequences of other heavy and light chain variable regions to determine the regions of sequence hypervariability. Using routine recombinant DNA techniques, one or more of the CDRs may be inserted within framework regions, e.g., into human framework regions to humanize a non-human antibody, as described supra. The framework regions may be naturally occurring or consensus framework regions, and preferably human framework regions (see, e.g., Chothia et al., J. Mol. Biol. 278: 457-479 (1998) for a listing of human framework regions). Preferably, the polynucleotide generated by the combination of the framework regions and CDRs encodes an antibody that specifically binds a polypeptide of the invention. Preferably, as discussed supra, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

In addition, techniques developed for the production of “chimeric antibodies” (Morrison et al., Proc. Natl. Acad. Sci. 81:851-855 (1984); Neuberger et al., Nature 312:604-608 (1984); Takeda et al., Nature 314:452-454 (1985)) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human immunoglobulin constant region, e.g., humanized antibodies.

Alternatively, techniques described for the production of single chain antibodies (U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989)) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli may also be used (Skerra et al., Science 242:1038-1041 (1988)).

More preferably, a clone encoding an antibody of the present invention may be obtained according to the method described in the Example section herein.

### Methods of Producing Antibodies

The antibodies of the invention can be produced by any method known in the , art for the synthesis of antibodies, in particular, by chemical synthesis or preferably, by recombinant expression techniques.

Recombinant expression of an antibody of the invention, or fragment, derivative or analog thereof, (e.g., a heavy or light chain of an antibody of the invention or a single chain antibody of the invention), requires construction of an expression vector containing a polynucleotide that encodes the antibody. Once a polynucleotide encoding an antibody molecule or a heavy or light chain of an antibody, or portion thereof (preferably containing the heavy or light chain variable domain), of the invention has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, or a heavy or light chain thereof, or a heavy or light chain variable domain, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy or light chain.

The expression vector is transferred to a host cell by conventional techniques and the transfected cells are then cultured by conventional techniques to produce an antibody of the invention. Thus, the invention includes host cells containing a polynucleotide encoding an antibody of the invention, or a heavy or light chain thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (1990)).

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited, to the E. coli expression vector pUR278 (Ruther et al., EMBO J. 2:1791 (1983)), in which the antibody coding sequence may be ligated individually into the vector in frame with the lac Z coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione S-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. (e.g., see Logan & Shenk, Proc. Natl. Acad. Sci. USA 81:355-359 (1984)). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 153:51-544 (1987)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, WT38, and in particular, breast cancer cell lines such as, for example, BT483, Hs578T, HTB2, BT20 and T47D, and normal mammary gland cell line such as, for example, CRL7030 and Hs578Bst.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compounds that interact directly or indirectly with the antibody molecule.

A number of selection systems may be used, including but not limited to the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:202 (1992)), and adenine phosphoribosyltransferase (Lowy et al., Cell 22:817 (1980)) genes can be employed in tk-, hgprt- or aprt- cells, respectively. Also, antimetabolite resistance can be used as the basis of selection for the following genes: dhfr, which confers resistance to methotrexate (Wigler et al., Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 Clinical Pharmacy 12:488-505; Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, 1993, TIB TECH 11(5):155-215); and hygro, which confers resistance to hygromycin (Santerre et al., Gene 30:147 (1984)). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., J. Mol. Biol. 150:1 (1981), which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol.3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., Mol. Cell. Biol. 3:257 (1983)).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, Nature 322:52 (1986); Köhler, Proc. Natl. Acad. Sci. USA 77:2197 (1980)). The coding sequences for the heavy and light chains may comprise cDNA or genomic DNA.

Once an antibody molecule of the invention has been produced by an animal, chemically synthesized, or recombinantly expressed, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. In addition, the antibodies of the present invention or fragments thereof can be fused to heterologous polypeptide sequences described herein or otherwise known in the art, to facilitate purification.

The present invention encompasses antibodies recombinantly fused or chemically conjugated (including both covalently and non-covalently conjugations) to a polypeptide (or portion thereof, preferably at least 10, 20, 30, 40, 50, 60, 70, 80, 90 or 100 amino acids of the polypeptide) of the present invention to generate fusion proteins. The fusion does not necessarily need to be direct, but may occur through linker sequences. The antibodies may be specific for antigens other than polypeptides (or portion thereof, preferably at least 10, 20, 30, 40, 50, 60, 70, 80, 90 or 100 amino acids of the polypeptide) of the present invention. For example, antibodies may be used to target the polypeptides of the present invention to particular cell types, either in vitro or in vivo, by fusing or conjugating the polypeptides of the present invention to antibodies specific for particular cell surface receptors. Antibodies fused or conjugated to the polypeptides of the present invention may also be used in in vitro immunoassays and purification methods using methods known in the art. See e.g., Harbor et al., supra, and PCT publication WO 93/21232; EP 439,095; Naramura et al., Immunol. Lett. 39:91-99 (1994); U.S. Pat. No. 5,474,981; Gillies et al., PNAS 89:1428-1432 (1992); Fell et al., J. Immunol. 146:2446-2452(1991), which are incorporated by reference in their entireties.

The present invention further includes compositions comprising the polypeptides of the present invention fused or conjugated to antibody domains other than the variable regions. For example, the polypeptides of the present invention may be fused or conjugated to an antibody Fc region, or portion thereof. The antibody portion fused to a polypeptide of the present invention may comprise the constant region, hinge region, CH1 domain, CH2 domain, and CH3 domain or any combination of whole domains or portions thereof. The polypeptides may also be fused or conjugated to the above antibody portions to form multimers. For example, Fc portions fused to the polypeptides of the present invention can form dimers through disulfide bonding between the Fc portions. Higher multimeric forms can be made by fusing the polypeptides to portions of IgA and IgM. Methods for fusing or conjugating the polypeptides of the present invention to antibody portions are known in the art. See, e.g., U.S. Pat. Nos. 5,336,603; 5,622,929; 5,359,046; 5,349,053; 5,447,851; 5,112,946; EP 307,434; EP 367,166; PCT publications WO 96/04388; WO 91/06570; Ashkenazi et al., Proc. Natl. Acad. Sci. USA 88:10535-10539 (1991); Zheng et al., J. Inmunol. 154:5590-5600 (1995); and Vil et al., Proc. Natl. Acad. Sci. USA 89:11337-11341(1992) (said references incorporated by reference in their entireties).

As discussed, supra, the polypeptides corresponding to a polypeptide, polypeptide fragment, or a variant of SEQ ID NO:6, 8, 10, 12, 14, 16, 18, 20, 22, 24, 26, 28, 30, 32, 34, 36, 38, 40, 42, 44, 46, 48, 50, and/or 604 may be fused or conjugated to the above antibody portions to increase the in vivo half life of the polypeptides or for use in immunoassays using methods known in the art. Further, the polypeptides corresponding to SEQ ID NO:6, 8, 10, 12, 14, 16, 18, 20, 22, 24, 26, 28, 30, 32, 34, 36, 38, 40, 42, 44, 46, 48, 50, and/or 604 may be fused or conjugated to the above antibody portions to facilitate purification. One reported example describes chimeric proteins consisting of the first two domains of the human CD4-polypeptide and various domains of the constant regions of the heavy or light chains of mammalian immunoglobulins. (EP 394,827; Traunecker et al., Nature 331:84-86 (1988). The polypeptides of the present invention fused or conjugated to an antibody having disulfide-linked dimeric structures (due to the IgG) may also be more efficient in binding and neutralizing other molecules, than the monomeric secreted protein or protein fragment alone. (Fountoulakis et al., J. Biochem. 270:3958-3964 (1995)). In many cases, the Fc part in a fusion protein is beneficial in therapy and diagnosis, and thus can result in, for example, improved pharmacokinetic properties. (EP A 232,262). Alternatively, deleting the Fc part after the fusion protein has been expressed, detected, and purified, would be desired. For example, the Fc portion may hinder therapy and diagnosis if the fusion protein is used as an antigen for immunizations. In drug discovery, for example, human proteins, such as hIL-5, have been fused with Fc portions for the purpose of high-throughput screening assays to identify antagonists of hIL-5. (See, Bennett et al., J. Molecular Recognition 8:52-58 (1995); Johanson et al., J. Biol. Chem. 270:9459-9471 (1995).

Moreover, the antibodies or fragments thereof of the present invention can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., Cell 37:767 (1984)) and the “flag” tag.

The present invention further encompasses antibodies or fragments thereof conjugated to a diagnostic or therapeutic agent. The antibodies can be used diagnostically to, for example, monitor the development or progression of a tumor as part of a clinical testing procedure to, e.g., determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. The detectable substance may be coupled or conjugated either directly to the antibody (or fragment thereof) or indirectly, through an intermediate (such as, for example, a linker known in the art) using techniques known in the art. See, for example, U.S. Pat. No. 4,741,900 for metal ions which can be conjugated to antibodies for use as diagnostics according to the present invention. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylaamine fluorescein, dansyl chloride or phycoerythrin; an example of a luminescent material includes luminol; examples of bioluminescent materials include luciferase, luciferin, and aequorin; and examples of suitable radioactive material include 125I, 131I, 111In or 99Tc.

Further, an antibody or fragment thereof may be conjugated to a therapeutic moiety such as a cytotoxin, e.g., a cytostatic or cytocidal agent, a therapeutic agent or a radioactive metal ion, e.g., alpha-emitters such as, for example, 213Bi. A cytotoxin or cytotoxic agent includes any agent that is detrimental to cells. Examples include paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologues thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (TI) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, a-interferon, β-interferon, nerve growth factor, platelet derived growth factor, tissue plasminogen activator, an apoptotic agent, e.g., TNF-alpha, TNF-beta, AIM I (See, International Publication No. WO 97/33899), AIM II (See, International Publication No. WO 97/34911), Fas Ligand (Takahashi et al., Int. Immunol., 6:1567-1574 (1994)), VEGI (See, International Publication No. WO 99/23105), a thrombotic agent or an anti-angiogenic agent, e.g., angiostatin or endostatin; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev. 62:119-58 (1982).

Alternatively, an antibody can be conjugated to a second antibody to form an antibody heteroconjugate as described by Segal in U.S. Pat. No. 4,676,980, which is incorporated herein by reference in its entirety.

An antibody, with or without a therapeutic moiety conjugated to it, administered alone or in combination with cytotoxic factor(s) and/or cytokine(s) can be used as a therapeutic.

The present invention also encompasses the creation of synthetic antibodies directed against the polypeptides of the present invention. One example of synthetic antibodies is described in Radrizzani, M., et al., Medicina, (Aires), 59(6):753-8, (1999)). Recently, a new class of synthetic antibodies has been described and are referred to as molecularly imprinted polymers (MIPs) (Semorex, Inc.). Antibodies, peptides, and enzymes are often used as molecular recognition elements in chemical and biological sensors. However, their lack of stability and signal transduction mechanisms limits their use as sensing devices. Molecularly imprinted polymers (MIPs) are capable of mimicking the function of biological receptors but with less stability constraints. Such polymers provide high sensitivity and selectivity while maintaining excellent thermal and mechanical stability. MIPs have the ability to bind to small molecules and to target molecules such as organics and proteins' with equal or greater potency than that of natural antibodies. These “super” MIPs have higher affinities for their target and thus require lower concentrations for efficacious binding.

During synthesis, the MIPs are imprinted so as to have complementary size, shape, charge and functional groups of the selected target by using the target molecule itself (such as a polypeptide, antibody, etc.), or a substance having a very similar structure, as its “print” or “template.” MIPs can be derivatized with the same reagents afforded to antibodies. For example, fluorescent ‘super’ MIPs can be coated onto beads or wells for use in highly sensitive separations or assays, or for use in high throughput screening of proteins.

Moreover, MIPs based upon the structure of the polypeptide(s) of the present invention may be useful in screening for compounds that bind to the polypeptide(s) of the invention. Such a MIP would serve the role of a synthetic “receptor” by minimicking the native architecture of the polypeptide. In fact, the ability of a MIP to serve the role of a synthetic receptor has already been demonstrated for the estrogen receptor (Ye, L., Yu, Y., Mosbach, K, Analyst., 126(6):760-5, (2001); Dickert, F, L., Hayden, O., Halikias, K, P, Analyst., 126(6):766-71, (2001)). A synthetic receptor may either be mimicked in its entirety (e.g., as the entire protein), or mimicked as a series of short peptides corresponding to the protein (Rachkov, A., Minoura, N, Biochim, Biophys, Acta., 1544(1-2):255-66, (2001)). Such a synthetic receptor MIPs may be employed in any one or more of the screening methods described elsewhere herein.

MIPs have also been shown to be useful in “sensing” the presence of its mimicked molecule (Cheng, Z., Wang, E., Yang, X, Biosens, Bioelectron., 16(3):179-85, (2001); Jenkins, A, L., Yin, R., Jensen, J. L, Analyst., 126(6):798-802, (2001); Jenkins, A, L., Yin, R., Jensen, J. L, Analyst., 126(6):798-802, (2001)). For example, a MIP designed using a polypeptide of the present invention may be used in assays designed to identify, and potentially quantitate, the level of said polypeptide in a sample. Such a MIP may be used as a substitute for any component described in the assays, or kits, provided herein (e.g., ELISA, etc.).

A number of methods may be employed to create MIPs to a specific receptor, ligand, polypeptide, peptide, organic molecule. Several preferred methods are described by Esteban et al in J. Anal, Chem., 370(7):795-802, (2001), which is hereby incorporated herein by reference in its entirety in addition to any references cited therein. Additional methods are known in the art and are encompassed by the present invention, such as for example, Hart, B, R., Shea, K, J. J. Am. Chem, Soc., 123(9):2072-3, (2001); and Quaglia, M., Chenon, K., Hall, A, J., De, Lorenzi, E., Sellergren, B, J. Am. Chem, Soc., 123(10):2146-54, (2001); which are hereby incorporated by reference in their entirety herein.

### Uses for Antibodies Directed Against Polypeptides of the Invention

The antibodies of the present invention have various utilities. For example, such antibodies may be used in diagnostic assays to detect the presence or quantification of the polypeptides of the invention in a sample. Such a diagnostic assay may be comprised of at least two steps. The first, subjecting a sample with the antibody, wherein the sample is a tissue (e.g., human, animal, etc.), biological fluid (e.g., blood, urine, sputum, semen, amniotic fluid, saliva, etc.), biological extract (e.g., tissue or cellular homogenate, etc.), a protein microchip (e.g., See Arenkov P, et al., Anal Biochem., 278(2):123-131 (2000)), or a chromatography column, etc. And a second step involving the quantification of antibody bound to the substrate. Alternatively, the method may additionally involve a first step of attaching the antibody, either covalently, electrostatically, or reversibly, to a solid support, and a second step of subjecting the bound antibody to the sample, as defined above and elsewhere herein.

Various diagnostic assay techniques are known in the art, such as competitive binding assays, direct or indirect sandwich assays and immunoprecipitation assays conducted in either heterogeneous or homogenous phases (Zola, Monoclonal Antibodies: A Manual of Techniques, CRC Press, Inc., (1987), pp147-158). The antibodies used in the diagnostic assays can be labeled with a detectable moiety. The detectable moiety should be capable of producing, either directly or indirectly, a detectable signal. For example, the detectable moiety may be a radioisotope, such as 2H, 14C, 32P, or 125I, a florescent or chemiluminescent compound, such as fluorescein isothiocyanate, rhodamine, or luciferin, or an enzyme, such as alkaline phosphatase, beta-galactosidase, green fluorescent protein, or horseradish peroxidase. Any method known in the art for conjugating the antibody to the detectable moiety may be employed, including those methods described by Hunter et al., Nature, 144:945 (1962); Dafvid et al., Biochem., 13:1014 (1974); Pain et al., J. Immunol. Metho., 40:219(1981); and Nygren, J. Histochem. And Cytochem., 30:407 (1982).

Antibodies directed against the polypeptides of the present invention are useful for the affinity purification of such polypeptides from recombinant cell culture or natural sources. In this process, the antibodies against a particular polypeptide are immobilized on a suitable support, such as a Sephadex resin or filter paper, using methods well known in the art. The immobilized antibody then is contacted with a sample containing the polypeptides to be purified, and thereafter the support is washed with a suitable solvent that will remove substantially all the material in the sample except for the desired polypeptides, which are bound to the immobilized antibody. Finally, the support is washed with another suitable solvent that will release the desired polypeptide from the antibody.

### Immunophenotyping

The antibodies of the invention may be utilized for immunophenotyping of cell lines and biological samples. The translation product of the gene of the present invention may be useful as a cell specific marker, or more specifically as a cellular marker that is differentially expressed at various stages of differentiation and/or maturation of particular cell types. Monoclonal antibodies directed against a specific epitope, or combination of epitopes, will allow for the screening of cellular populations expressing the marker. Various techniques can be utilized using monoclonal antibodies to screen for cellular populations expressing the marker(s), and include magnetic separation using antibody-coated magnetic beads, “panning” with antibody attached to a solid matrix (i.e., plate), and flow cytometry (See, e.g., U.S. Pat. No. 5,985,660; and Morrison et al., Cell, 96:737-49 (1999)).

These techniques allow for the screening of particular populations of cells, such as might be found with hematological malignancies (i.e. minimal residual disease (MRD) in acute leukemic patients) and “non-self” cells in transplantations to prevent Graft-versus-Host Disease (GVHD). Alternatively, these techniques allow for the screening of hematopoietic stem and progenitor cells capable of undergoing proliferation and/or differentiation, as might be found in human umbilical cord blood.

### Assays for Antibody Binding

The antibodies of the invention may be assayed for immunospecific binding by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al, eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., 32P or 125I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1.

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al, eds, 1994, Current Protocols in Molecular Biology, Vol. 1, John Wiley & Sons, Inc., New York at 11.2.1.

The binding affinity of an antibody to an antigen and the off-rate of an antibody-antigen interaction can be determined by competitive binding assays. One example of a competitive binding assay is a radioimmunoassay comprising the incubation of labeled antigen (e.g., 3H or 125I) with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound (e.g., 3H or 125I) in the presence of increasing amounts of an unlabeled second antibody.

### Therapeutic Uses of Antibodies

The present invention is further directed to antibody-based therapies which involve administering antibodies of the invention to an animal, preferably a mammal, and most preferably a human, patient for treating one or more of the disclosed diseases, disorders, or conditions. Therapeutic compounds of the invention include, but are not limited to, antibodies of the invention (including fragments, analogs and derivatives thereof as described herein) and nucleic acids encoding antibodies of the invention (including fragments, analogs and derivatives thereof and anti-idiotypic antibodies as described herein). The antibodies of the invention can be used to treat, inhibit or prevent diseases, disorders or conditions associated with aberrant expression and/or activity of a polypeptide of the invention, including, but not limited to, any one or more of the diseases, disorders, or conditions described herein. The treatment and/or prevention of diseases, disorders, or conditions associated with aberrant expression and/or activity of a polypeptide of the invention includes, but is not limited to, alleviating symptoms associated with those diseases, disorders or conditions. Antibodies of the invention may be provided in pharmaceutically acceptable compositions as known in the art or as described herein.

A summary of the ways in which the antibodies of the present invention may be used therapeutically includes binding polynucleotides or polypeptides of the present invention locally or systemically in the body or by direct cytotoxicity of the antibody, e.g. as mediated by complement (CDC) or by effector cells (ADCC). Some of these approaches are described in more detail below. Armed with the teachings provided herein, one of ordinary skill in the art will know how to use the antibodies of the present invention for diagnostic, monitoring or therapeutic purposes without undue experimentation.

The antibodies of this invention may be advantageously utilized in combination with other monoclonal or chimeric antibodies, or with lymphokines or hematopoietic growth factors (such as, e.g., IL-2, IL-3 and IL-7), for example, which serve to increase the number or activity of effector cells which interact with the antibodies.

The antibodies of the invention may be administered alone or in combination with other types of treatments (e.g., radiation therapy, chemotherapy, hormonal therapy, immunotherapy and anti-tumor agents). Generally, administration of products of a species origin or species reactivity (in the case of antibodies) that is the same species as that of the patient is preferred. Thus, in a preferred embodiment, human antibodies, fragments derivatives, analogs, or nucleic acids, are administered to a human patient for therapy or prophylaxis.

It is preferred to use high affinity and/or potent in vivo inhibiting and/or neutralizing antibodies against polypeptides or polynucleotides of the present invention, fragments or regions thereof, for both immunoassays directed to and therapy of disorders related to polynucleotides or polypeptides, including fragments thereof, of the present invention. Such antibodies, fragments, or regions, will preferably have an affinity for polynucleotides or polypeptides of the invention, including fragments thereof. Preferred binding affinities include those with a dissociation constant or Kd less than 5×10-2 M, 10-2 M, 5×10-3 M, 10-3 M, 5×10-4 M, 10-4 M, 5×10-5 M, 10-5 M, 5×10-6 M, 10-6 M, 5×10-7 M, 10-7 M, 5×10-8 M, 10-8 M, 5×10-9 M, 10-9 M, 5×10-10 M, 10-10 M, 5×10-11 M, 10-11 M, 5×10-12 M, 10-12 M, 5×10-13 M, 10-13 M, 5×10-14 M, 10-14 M, 5×10-15 M, and 10-15 M.

Antibodies directed against polypeptides of the present invention are useful for inhibiting allergic reactions in animals. For example, by administering a therapeutically acceptable dose of an antibody, or antibodies, of the present invention, or a cocktail of the present antibodies, or in combination with other antibodies of varying sources, the animal may not elicit an allergic response to antigens.

Likewise, one could envision cloning the gene encoding an antibody directed against a polypeptide of the present invention, said polypeptide having the potential to elicit an allergic and/or immune response in an organism, and transforming the organism with said antibody gene such that it is expressed (e.g., constitutively, inducibly, etc.) in the organism. Thus, the organism would effectively become resistant to an allergic response resulting from the ingestion or presence of such an immune/allergic reactive polypeptide. Moreover, such a use of the antibodies of the present invention may have particular utility in preventing and/or ameliorating autoimmune diseases and/or disorders, as such conditions are typically a result of antibodies being directed against endogenous proteins. For example, in the instance where the polypeptide of the present invention is responsible for modulating the immune response to auto-antigens, transforming the organism and/or individual with a construct comprising any of the promoters disclosed herein or otherwise known in the art, in addition, to a polynucleotide encoding the antibody directed against the polypeptide of the present invention could effective inhibit the organisms immune system from eliciting an immune response to the auto-antigen(s). Detailed descriptions of therapeutic and/or gene therapy applications of the present invention are provided elsewhere herein.

Alternatively, antibodies of the present invention could be produced in a plant (e.g., cloning the gene of the antibody directed against a polypeptide of the present invention, and transforming a plant with a suitable vector comprising said gene for constitutive expression of the antibody within the plant), and the plant subsequently ingested by an animal, thereby conferring temporary immunity to the animal for the specific antigen the antibody is directed towards (See, for example, U.S. Pat. Nos. 5,914,123 and 6,034,298).

In another embodiment, antibodies of the present invention, preferably polyclonal antibodies, more preferably monoclonal antibodies, and most preferably single-chain antibodies, can be used as a means of inhibiting gene expression of a particular gene, or genes, in a human, mammal, and/or other organism. See, for example, International Publication Number WO 00/05391, published Feb. 2, 2000 to Dow Agrosciences LLC. The application of such methods for the antibodies of the present invention are known in the art, and are more particularly described elsewhere herein.

In yet another embodiment, antibodies of the present invention may be useful for multimerizing the polypeptides of the present invention. For example, certain proteins may confer enhanced biological activity when present in a multimeric state (i.e., such enhanced activity may be due to the increased effective concentration of such proteins whereby more protein is available in a localized location).

### Antibody-Based Gene Therapy

In a specific embodiment, nucleic acids comprising sequences encoding antibodies or functional derivatives thereof, are administered to treat, inhibit or prevent a disease or disorder associated with aberrant expression and/or activity of a polypeptide of the invention, by way of gene therapy. Gene therapy refers to therapy performed by the administration to a subject of an expressed or expressible nucleic acid. In this embodiment of the invention, the nucleic acids produce their encoded protein that mediates a therapeutic effect.

Any of the methods for gene therapy available in the art can be used according to the present invention. Exemplary methods are described below.

For general reviews of the methods of gene therapy, see Goldspiel et al., Clinical Pharmacy 12:488-505 (1993); Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 May, 1993; TIBTECH 11(5):155-215 (1993). Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, the compound comprises nucleic acid sequences encoding an antibody, said nucleic acid sequences being part of expression vectors that express the antibody or fragments or chimeric proteins or heavy or light chains thereof in a suitable host. In particular, such nucleic acid sequences have promoters operably linked to the antibody coding region, said promoter being inducible or constitutive, and, optionally, tissue-specific. In another particular embodiment, nucleic acid molecules are used in which the antibody coding sequences and any other desired sequences are flanked by regions that promote homologous recombination at a desired site in the genome, thus providing for intrachromosomal expression of the antibody encoding nucleic acids (Koller and Smithies, Proc. Natl. Acad. Sci. USA 86:8932-8935 (1989); Zijlstra et al., Nature 342:435-438 (1989). In specific embodiments, the expressed antibody molecule is a single chain antibody; alternatively, the nucleic acid sequences include sequences encoding both the heavy and light chains, or fragments thereof, of the antibody.

Delivery of the nucleic acids into a patient may be either direct, in which case the patient is directly exposed to the nucleic acid or nucleic acid-carrying vectors, or indirect, in which case, cells are first transformed with the nucleic acids in vitro, then transplanted into the patient. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, J. Biol. Chem. 262:4429-4432 (1987)) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; WO92/20316; WO93/14188, WO 93/20221). Alternatively, the nucleic acid can be introduced intracellularly and incorporated within host cell DNA for expression, by homologous recombination (Koller and Smithies, Proc. Natl. Acad. Sci. USA 86:8932-8935 (1989); Zijlstra et al., Nature 342:435-438 (1989)).

In a specific embodiment, viral vectors that contains nucleic acid sequences encoding an antibody of the invention are used. For example, a retroviral vector can be used (see Miller et al., Meth. Enzymol. 217:581-599 (1993)). These retroviral vectors contain the components necessary for the correct packaging of the viral genome and integration into the host cell DNA. The nucleic acid sequences encoding the antibody to be used in gene therapy are cloned into one or more vectors, which facilitates delivery of the gene into a patient. More detail about retroviral vectors can be found in Boesen et al., Biotherapy 6:291-302 (J994), which describes the use of a retroviral vector to deliver the mdr1 gene to hematopoietic stem cells in order to make the stem cells more resistant to chemotherapy. Other references illustrating the use of retroviral vectors in gene therapy are: Clowes et al., J. Clin. Invest. 93:644-651 (1994); Kiem et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993).

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993) present a review of adenovirus-based gene therapy. Bout et al., Human Gene Therapy 5:3-10 (1994) demonstrated the use of adenovirus vectors to transfer genes to the respiratory epithelia of rhesus monkeys. Other instances of the use of adenoviruses in gene therapy can be found in Rosenfeld et al., Science 252:431-434 (1991); Rosenfeld et al., Cell 68:143-155 (1992); Mastrangeli et al., J. Clin. Invest. 91:225-234 (1993); PCT Publication WO94/12649; and Wang, et al., Gene Therapy 2:775-783 (1995). In a preferred embodiment, adenovirus vectors are used.

Adeno-associated virus (AAV) has also been proposed for use in gene therapy (Walsh et al., Proc. Soc. Exp. Biol. Med. 204:289-300 (1993); U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, Meth. Enzymol. 217:599-618 (1993); Cohen et al., Meth. Enzymol. 217:618-644 (1993); Cline, Pharmac. Ther. 29:69-92m (1985) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as Tlymphocytes, Blymphocytes, monocytes, macrophages, neutrophils, cosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the patient.

In an embodiment in which recombinant cells are used in gene therapy, nucleic acid sequences encoding an antibody are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (see e.g. PCT Publication WO 94/08598; Stemple and Anderson, Cell 71:973-985 (1992); Rheinwald, Meth. Cell Bio. 21A:229 (1980); and Pittelkow and Scott, Mayo Clinic Proc. 61:771 (1986)).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription. Demonstration of Therapeutic or Prophylactic Activity The compounds or pharmaceutical compositions of the invention are preferably tested in vitro, and then in vivo for the desired therapeutic or prophylactic activity, prior to use in humans. For example, in vitro assays to demonstrate the therapeutic or prophylactic utility of a compound or pharmaceutical composition include, the effect of a compound on a cell line or a patient tissue sample. The effect of the compound or composition on the cell line and/or tissue sample can be determined utilizing techniques known to those of skill in the art including, but not limited to, rosette formation assays and cell lysis assays. In accordance with the invention, in vitro assays which can be used to determine whether administration of a specific compound is indicated, include in vitro cell culture assays in which a patient tissue sample is grown in culture, and exposed to or otherwise administered a compound, and the effect of such compound upon the tissue sample is observed.

### Therapeutic/Prophylactic Administration and Compositions

The invention provides methods of treatment, inhibition and prophylaxis by administration to a subject of an effective amount of a compound or pharmaceutical composition of the invention, preferably an antibody of the invention. In a preferred aspect, the compound is substantially purified (e.g., substantially free from substances that limit its effect or produce undesired side-effects). The subject is preferably an animal, including but not limited to animals such as cows, pigs, horses, chickens, cats, dogs, etc., and is preferably a mammal, and most preferably human.

Formulations and methods of administration that can be employed when the compound comprises a nucleic acid or an immunoglobulin are described above; additional appropriate formulations and routes of administration can be selected from among those described herein below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu, J. Biol. Chem. 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the invention, care must be taken to use materials to which the protein does not absorb.

In another embodiment, the compound or composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.) In yet another embodiment, the compound or composition can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)).

Other controlled release systems are discussed in the review by Langer (Science 249:1527-1533 (1990)).

In a specific embodiment where the compound of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA 88:1864-1868 (1991)), etc. Alternatively, a nucleic acid can be introduced intracellularly and incorporated within host cell DNA for expression, by homologous recombination.

The present invention also provides pharmaceutical compositions. Such compositions comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of the compound of the invention which will be effective in the treatment, inhibition and prevention of a disease or disorder associated with aberrant expression and/or activity of a polypeptide of the invention can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

For antibodies, the dosage administered to a patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention may be reduced by enhancing uptake and tissue penetration (e.g., into the brain) of the antibodies by modifications such as, for example, lipidation.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

### Diagnosis and Imaging with Antibodies

Labeled antibodies, and derivatives and analogs thereof, which specifically bind to a polypeptide of interest can be used for diagnostic purposes to detect, diagnose, or monitor diseases, disorders, and/or conditions associated with the aberrant expression and/or activity of a polypeptide of the invention. The invention provides for the detection of aberrant expression of a polypeptide of interest, comprising (a) assaying the expression of the polypeptide of interest in cells or body fluid of an individual using one or more antibodies specific to the polypeptide interest and (b) comparing the level of gene expression with a standard gene expression level, whereby an increase or decrease in the assayed polypeptide gene expression level compared to the standard expression level is indicative of aberrant expression.

The invention provides a diagnostic assay for diagnosing a disorder, comprising (a) assaying the expression of the polypeptide of interest in cells or body fluid of an individual using one or more antibodies specific to the polypeptide interest and (b) comparing the level of gene expression with a standard gene expression level, whereby an increase or decrease in the assayed polypeptide gene expression level compared to the standard expression level is indicative of a particular disorder. With respect to cancer, the presence of a relatively high amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Antibodies of the invention can be used to assay protein levels in a biological sample using classical immunohistological methods known to those of skill in the art (e.g., see Jalkanen, et al., J. Cell. Biol. 101:976-985 (1985); Jalkanen, et al., J. Cell. Biol. 105:3087-3096 (1987)). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (125I, 121I), carbon (14C), sulfur (35S), tritium (3H), indium (112In), and technetium (99Tc); luminescent labels, such as luminol; and fluorescent labels, such as fluorescein and rhodamine, and biotin.

One aspect of the invention is the detection and diagnosis of a disease or disorder associated with aberrant expression of a polypeptide of interest in an animal, preferably a mammal and most preferably a human. In one embodiment, diagnosis comprises: a) administering (for example, parenterally, subcutaneously, or intraperitoneally) to a subject an effective amount of a labeled molecule which specifically binds to the polypeptide of interest; b) waiting for a time interval following the administering for permitting the labeled molecule to preferentially concentrate at sites in the subject where the polypeptide is expressed (and for unbound labeled molecule to be cleared to background level); c) determining background level; and d) detecting the labeled molecule in the subject, such that detection of labeled molecule above the background level indicates that the subject has a particular disease or disorder associated with aberrant expression of the polypeptide of interest. Background level can be determined by various methods including, comparing the amount of labeled molecule detected to a standard value previously determined for a particular system.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of 99mTc. The labeled antibody or antibody fragment will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).

Depending on several variables, including the type of label used and the mode of administration, the time interval following the administration for permitting the labeled molecule to preferentially concentrate at sites in the subject and for unbound labeled molecule to be cleared to background level is 6 to 48 hours or 6 to 24 hours or 6 to 12 hours. In another embodiment the time interval following administration is 5 to 20 days or 5 to 10 days.

In an embodiment, monitoring of the disease or disorder is carried out by repeating the method for diagnosing the disease or disease, for example, one month after initial diagnosis, six months after initial diagnosis, one year after initial diagnosis, etc.

Presence of the labeled molecule can be detected in the patient using methods known in the art for in vivo scanning. These methods depend upon the type of label used. Skilled artisans will be able to determine the appropriate method for detecting a particular label. Methods and devices that may be used in the diagnostic methods of the invention include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patent using positron emission-tomography. In yet another embodiment, the molecule is labeled with a paramagnetic label and is detected in a patient using magnetic resonance imaging (MRI).

### Kits

The present invention provides kits that can be used in the above methods. In one embodiment, a kit comprises an antibody of the invention, preferably a purified antibody, in one or more containers. In a specific embodiment, the kits of the present invention contain a substantially isolated polypeptide comprising an epitope which is specifically immunoreactive with an antibody included in the kit. Preferably, the kits of the present invention further comprise a control antibody which does not react with the polypeptide of interest. In another specific embodiment, the kits of the present invention contain a means for detecting the binding of an antibody to a polypeptide of interest (e.g., the antibody may be conjugated to a detectable substrate such as a fluorescent compound, an enzymatic substrate, a radioactive compound or a luminescent compound, or a second antibody which recognizes the first antibody may be conjugated to a detectable substrate).

In another specific embodiment of the present invention, the kit is a diagnostic kit for use in screening serum containing antibodies specific against proliferative and/or cancerous polynucleotides and polypeptides. Such a kit may include a control antibody that does not react with the polypeptide of interest. Such a kit may include a substantially isolated polypeptide antigen comprising an epitope which is specifically immunoreactive with at least one anti-polypeptide antigen antibody. Further, such a kit includes means for detecting the binding of said antibody to the antigen (e.g., the antibody may be conjugated to a fluorescent compound such as fluorescein or rhodamine which can be detected by flow cytometry). In specific embodiments, the kit may include a recombinantly produced or chemically synthesized polypeptide antigen. The polypeptide antigen of the kit may also be attached to a solid support.

In a more specific embodiment the detecting means of the above-described kit includes a solid support to which said polypeptide antigen is attached. Such a kit may also include a non-attached reporter-labeled anti-human antibody. In this embodiment, binding of the antibody to the polypeptide antigen can be detected by binding of the said reporter-labeled antibody.

In an additional embodiment, the invention includes a diagnostic kit for use in screening serum containing antigens of the polypeptide of the invention. The diagnostic kit includes a substantially isolated antibody specifically immunoreactive with polypeptide or polynucleotide antigens, and means for detecting the binding of the polynucleotide or polypeptide antigen to the antibody. In one embodiment, the antibody is attached to a solid support. In a specific embodiment, the antibody may be a monoclonal antibody. The detecting means of the kit may include a second, labeled monoclonal antibody. Alternatively, or in addition, the detecting means may include a labeled, competing antigen.

In one diagnostic configuration, test serum is reacted with a solid phase reagent having a surface-bound antigen obtained by the methods of the present invention. After binding with specific antigen antibody to the reagent and removing unbound serum components by washing, the reagent is reacted with reporter-labeled anti-human antibody to bind reporter to the reagent in proportion to the amount of bound anti-antigen antibody on the solid support. The reagent is again washed to remove unbound labeled antibody, and the amount of reporter associated with the reagent is determined. Typically, the reporter is an enzyme which is detected by incubating the solid phase in the presence of a suitable fluorometric, luminescent or calorimetric substrate (Sigma, St. Louis, Mo.).

The solid surface reagent in the above assay is prepared by known techniques for attaching protein material to solid support material, such as polymeric beads, dip sticks, 96-well plate or filter material. These attachment methods generally include non-specific adsorption of the protein to the support or covalent attachment of the protein, typically through a free amine group, to a chemically reactive group on the solid support, such as an activated carboxyl, hydroxyl, or aldehyde group. Alternatively, streptavidin coated plates can be used in conjunction with biotinylated antigen(s).

Thus, the invention provides an assay system or kit for carrying out this diagnostic method. The kit generally includes a support with surface-bound recombinant antigens, and a reporter-labeled anti-human antibody for detecting surface-bound anti-antigen antibody.

### Fusion Proteins

Any polypeptide of the present invention can be used to generate fusion proteins. For example, the polypeptide of the present invention, when fused to a second protein, can be used as an antigenic tag. Antibodies raised against the polypeptide of the present invention can be used to indirectly detect the second protein by binding to the polypeptide. Moreover, because certain proteins target cellular locations based on trafficking signals, the polypeptides of the present invention can be used as targeting molecules once fused to other proteins.

Examples of domains that can be fused to polypeptides of the present invention include not only heterologous signal sequences, but also other heterologous functional regions. The fusion does not necessarily need to be direct, but may occur through linker sequences.

Moreover, fusion proteins may also be engineered to improve characteristics of the polypeptide of the present invention. For instance, a region of additional amino acids, particularly charged amino acids, may be added to the N-terminus of the polypeptide to improve stability and persistence during purification from the host cell or subsequent handling and storage. Peptide moieties may be added to the polypeptide to facilitate purification. Such regions may be removed prior to final preparation of the polypeptide. Similarly, peptide cleavage sites can be introduced in-between such peptide moieties, which could additionally be subjected to protease activity to remove said peptide(s) from the protein of the present invention. The addition of peptide moieties, including peptide cleavage sites, to facilitate handling of polypeptides are familiar and routine techniques in the art.

Moreover, polypeptides of the present invention, including fragments, and specifically epitopes, can be combined with parts of the constant domain of immunoglobulins (IgA, IgE, IgG, IgM) or portions thereof (CH1, CH2, CH3, and any combination thereof, including both entire domains and portions thereof), resulting in chimeric polypeptides. These fusion proteins facilitate purification and show an increased half-life in vivo. One reported example describes chimeric proteins consisting of the first two domains of the human CD4-polypeptide and various domains of the constant regions of the heavy or light chains of mammalian immunoglobulins. (EP A 394,827; Traunecker et al., Nature 331:84-86 (1988).) Fusion proteins having disulfide-linked dimeric structures (due to the IgG) can also be more efficient in binding and neutralizing other molecules, than the monomeric secreted protein or protein fragment alone. (Fountoulakis et al., J. Biochem. 270:3958-3964 (1995).)

Similarly, EP-A-O 464 533 (Canadian counterpart 2045869) discloses fusion proteins comprising various portions of the constant region of immunoglobulin molecules together with another human protein or part thereof. In many cases, the Fc part in a fusion protein is beneficial in therapy and diagnosis, and thus can result in, for example, improved pharmacokinetic properties. (EP-A 0232 262.) Alternatively, deleting the Fc part after the fusion protein has been expressed, detected, and purified, would be desired. For example, the Fc portion may hinder therapy and diagnosis if the fusion protein is used as an antigen for immunizations. In drug discovery, for example, human proteins, such as hIL-5, have been fused with Fc portions for the purpose of high-throughput screening assays to identify antagonists of hIL-5. (See, D. Bennett et al., J. Molecular Recognition 8:52-58 (1995); K. Johanson et al., J. Biol. Chem. 270:9459-9471 (1995).)

Moreover, the polypeptides of the present invention can be fused to marker sequences (also referred to as “tags”). Due to the availability of antibodies specific to such “tags”, purification of the fused polypeptide of the invention, and/or its identification is significantly facilitated since antibodies specific to the polypeptides of the invention are not required. Such purification may be in the form of an affinity purification whereby an anti-tag antibody or another type of affinity matrix (e.g., anti-tag antibody attached to the matrix of a flow-thru column) that binds to the epitope tag is present. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Another peptide tag useful for purification, the “HA” tag, corresponds to an epitope derived from the influenza hemagglutinin protein. (Wilson et al., Cell 37:767 (1984)).

The skilled artisan would acknowledge the existence of other “tags” which could be readily substituted for the tags referred to supra for purification and/or identification of polypeptides of the present invention (Jones C., et al., J Chromatogr A. 707(1):3-22 (1995)). For example, the c-myc tag and the 8F9, 3C7, 6E10, G4m B7 and 9E10 antibodies thereto (Evan et al., Molecular and Cellular Biology 5:3610-3616 (1985)); the Herpes Simplex virus glycoprotein D (gD) tag and its antibody (Paborsky et al., Protein Engineering, 3(6):547-553 (1990), the Flag-peptide—i.e., the octapeptide sequence DYKDDDDK (SEQ ID NO:605), (Hopp et al., Biotech. 6:1204-1210 (1988); the KT3 epitope peptide (Martin et al., Science, 255:192-194 (1992)); a-tubulin epitope peptide (Skinner et al., J. Biol. Chem., 266:15136-15166, (1991)); the T7 gene 10 protein peptide tag (Lutz-Freyermuth et al., Proc. Natl. Sci. USA, 87:6363-6397 (1990)), the FITC epitope (Zymed, Inc.), the GFP epitope (Zymed, Inc.), and the Rhodamine epitope (Zymed, Inc.).

The present invention also encompasses the attachment of up to nine codons encoding a repeating series of up to nine arginine amino acids to the coding region of a polynucleotide of the present invention. The invention also encompasses chemically derivitizing a polypeptide of the present invention with a repeating series of up to nine arginine amino acids. Such a tag, when attached to a polypeptide, has recently been shown to serve as a universal pass, allowing compounds access to the interior of cells without additional derivitization or manipulation (Wender, P., et al., unpublished data).

Protein fusions involving polypeptides of the present invention, including fragments and/or variants thereof, can be used for the following, non-limiting examples, subcellular localization of proteins, determination of protein-protein interactions via immunoprecipitation, purification of proteins via affinity chromatography, functional and/or structural characterization of protein. The present invention also encompasses the application of hapten specific antibodies for any of the uses referenced above for epitope fusion proteins. For example, the polypeptides of the present invention could be chemically derivatized to attach hapten molecules (e.g., DNP, (Zymed, Inc.)). Due to the availability of monoclonal antibodies specific to such haptens, the protein could be readily purified using immunoprecipation, for example.

Polypeptides of the present invention, including fragments and/or variants thereof, in addition to, antibodies directed against such polypeptides, fragments, and/or variants, may be fused to any of a number of known, and yet to be determined, toxins, such as ricin, saporin (Mashiba H, et al., Ann. N. Y. Acad. Sci. 1999;886:233-5), or HC toxin (Tonukari N J, et al., Plant Cell. 2000 February;12(2):237-248), for example. Such fusions could be used to deliver the toxins to desired tissues for which a ligand or a protein capable of binding to the polypeptides of the invention exists.

The invention encompasses the fusion of antibodies directed against polypeptides of the present invention, including variants and fragments thereof, to said toxins for delivering the toxin to specific locations in a cell, to specific tissues, and/or to specific species. Such bifunctional antibodies are known in the art, though a review describing additional advantageous fusions, including citations for methods of production, can be found in P. J. Hudson, Curr. Opp. In. Imm. 11:548-557, (1999); this publication, in addition to the references cited therein, are hereby incorporated by reference in their entirety herein. In this context, the term “toxin” may be expanded to include any heterologous protein, a small molecule, radionucleotides, cytotoxic drugs, liposomes, adhesion molecules, glycoproteins, ligands, cell or tissue-specific ligands, enzymes, of bioactive agents, biological response modifiers, anti-fungal agents, hormones, steroids, vitamins, peptides, peptide analogs, anti-allergenic agents, anti-tubercular agents, anti-viral agents, antibiotics, anti-protozoan agents, chelates, radioactive particles, radioactive ions, X-ray contrast agents, monoclonal antibodies, polyclonal antibodies and genetic material. In view of the present disclosure, one skilled in the art could determine whether any particular “toxin” could be used in the compounds of the present invention. Examples of suitable “toxins” listed above are exemplary only and are not intended to limit the “toxins” that may be used in the present invention.

Thus, any of these above fusions can be engineered using the polynucleotides or the polypeptides of the present invention.

### Vectors, Host Cells, and Protein Production

The present invention also relates to vectors containing the polynucleotide of the present invention, host cells, and the production of polypeptides by recombinant techniques. The vector may be, for example, a phage, plasmid, viral, or retroviral vector. Retroviral vectors may be replication competent or replication defective. In the latter case, viral propagation generally will occur only in complementing host cells.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The polynucleotide insert should be operatively linked to an appropriate promoter, such as the phage lambda PL promoter, the E. coli lac, trp, phoA and tac promoters, the SV40 early and late promoters and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, 293, and Bowes melanoma cells; and plant cells. Appropriate culture mediums and conditions for the above-described host cells are known in the art.

Among vectors preferred for use in bacteria include pQE70, pQE60 and pQE-9, available from QIAGEN, Inc.; pBluescript vectors, Phagescript vectors, pNH8A, pNH16a, pNH18A, pNH46A, available from Stratagene Cloning Systems, Inc.; and ptrc99a, pKK223-3, pKK233-3, pDR540, pRIT5 available from Pharmacia Biotech, Inc. Among preferred eukaryotic vectors are pWLNEO, pSV2CAT, pOG44, pXT1 and pSG available from Stratagene; and pSVK3, pBPV, pMSG and pSVL available from Pharmacia. Preferred expression vectors for use in yeast systems include, but are not limited to pYES2, pYD1, pTEF1/Zeo, pYES2/GS, pPICZ, pGAPZ, pGAPZalph, pPIC9, pPIC3.5, pHIL-D2, pHIL-S1, pPIC3.5K, pPIC9K, and PAO815 (all available from Invitrogen, Carlsbad, Calif.). Other suitable vectors will be readily apparent to the skilled artisan.

Introduction of the construct into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology (1986). It is specifically contemplated that the polypeptides of the present invention may in fact be expressed by a host cell lacking a recombinant vector.

A polypeptide of this invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification.

Polypeptides of the present invention, and preferably the secreted form, can also be recovered from: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect, and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

In one embodiment, the yeast Pichia pastoris is used to express the polypeptide of the present invention in a eukaryotic system. Pichia pastoris is a methylotrophic yeast which can metabolize methanol as its sole carbon source. A main step in the methanol metabolization pathway is the oxidation of methanol to formaldehyde using O2. This reaction is catalyzed by the enzyme alcohol oxidase. In order to metabolize methanol as its sole carbon source, Pichia pastoris must generate high levels of alcohol oxidase due, in part, to the relatively low affinity of alcohol oxidase for O2. Consequently, in a growth medium depending on methanol as a main carbon source, the promoter region of one of the two alcohol oxidase genes (AOX1) is highly active. In the presence of methanol, alcohol oxidase produced from the AOX1 gene comprises up to approximately 30% of the total soluble protein in Pichia pastoris. See, Ellis, S. B., et al., Mol. Cell. Biol. 5:1111-21 (1985); Koutz, P. J, et al., Yeast 5:167-77 (1989); Tschopp, J. F., et al., Nucd. Acids Res. 15:3859-76 (1987). Thus, a heterologous coding sequence, such as, for example, a polynucleotide of the present invention, under the transcriptional regulation of all or part of the AOX1 regulatory sequence is expressed at exceptionally high levels in Pichia yeast grown in the presence of methanol.

In one example, the plasmid vector pPIC9K is used to express DNA encoding a polypeptide of the invention, as set forth herein, in a Pichea yeast system essentially as described in “Pichia Protocols: Methods in Molecular Biology,” D. R. Higgins and J. Cregg, eds. The Humana Press, Totowa, N. J., 1998. This expression vector allows expression and secretion of a protein of the invention by virtue of the strong AOX1 promoter linked to the Pichia pastoris alkaline phosphatase (PHO) secretory signal peptide (i.e., leader) located upstream of a multiple cloning site.

Many other yeast vectors could be used in place of pPIC9K, such as, pYES2, pYD1, pTEF1/Zeo, pYES2/GS, pPICZ, pGAPZ, pGAPZalpha, pPIC9, pPIC3.5, pHIL-D2, pHIL-S1, pPIC3.5K, and PAO815, as one skilled in the art would readily appreciate, as long as the proposed expression construct provides appropriately located signals for transcription, translation, secretion (if desired), and the like, including an in-frame AUG, as required.

In another embodiment, high-level expression of a heterologous coding sequence, such as, for example, a polynucleotide of the present invention, may be achieved by cloning the heterologous polynucleotide of the invention into an expression vector such as, for example, pGAPZ or pGAPZalpha, and growing the yeast culture in the absence of methanol.

In addition to encompassing host cells containing the vector constructs discussed herein, the invention also encompasses primary, secondary, and immortalized host cells of vertebrate origin, particularly manunalian origin, that have been engineered to delete or replace endogenous genetic material (e.g., coding sequence), and/or to include genetic material (e.g., heterologous polynucleotide sequences) that is operably associated with the polynucleotides of the invention, and which activates, alters, and/or amplifies endogenous polynucleotides. For example, techniques known in the art may be used to operably associate heterologous control regions (e.g., promoter and/or enhancer) and endogenous polynucleotide sequences via homologous recombination, resulting in the formation of a new transcription unit (see, e.g., U.S. Pat. No. 5,641,670, issued Jun. 24, 1997; U.S. Pat. No. 5,733,761, issued Mar. 31, 1998; International Publication No. WO 96/29411, published Sep. 26, 1996; International Publication No. WO 94/12650, published Aug. 4, 1994; Koller et al., Proc. Natl. Acad. Sci. USA 86:8932-8935 (1989); and Zijlstra et al., Nature 342:435-438 (1989), the disclosures of each of which are incorporated by reference in their entireties).

In addition, polypeptides of the invention can be chemically synthesized using techniques known in the art (e.g., see Creighton, 1983, Proteins: Structures and Molecular Principles, W. H. Freeman & Co., N.Y., and Hunkapiller et al., Nature, 310:105-111 (1984)). For example, a polypeptide corresponding to a fragment of a polypeptide sequence of the invention can be synthesized by use of a peptide synthesizer. Furthermore, if desired, nonclassical amino acids or chemical amino acid analogs can be introduced as a substitution or addition into the polypeptide sequence. Non-classical amino acids include, but are not limited to, to the D-isomers of the common amino acids, 2,4-diaminobutyric acid, a-amino isobutyric acid, 4-aminobutyric acid, Abu, 2-amino butyric acid, g-Abu, e-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, b-alanine, fluoro-amino acids, designer amino acids such as b-methyl amino acids, Ca-methyl amino acids, Na-methyl amino acids, and amino acid analogs in general. Furthermore, the amino acid can be D (dextrorotary) or L (levorotary).

The invention encompasses polypeptides which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH4; acetylation, formylation, oxidation, reduction; metabolic synthesis in the presence of tunicamycin; etc.

Additional post-translational modifications encompassed by the invention include, for example, e.g., N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of prokaryotic host cell expression. The polypeptides may also be modified with a detectable label, such as an enzymatic, fluorescent, isotopic or affinity label to allow for detection and isolation of the protein, the addition of epitope tagged peptide fragments (e.g., FLAG, HA, GST, thioredoxin, maltose binding protein, etc.), attachment of affinity tags such as biotin and/or streptavidin, the covalent attachment of chemical moieties to the amino acid backbone, N- or C-terminal processing of the polypeptides ends (e.g., proteolytic processing), deletion of the N-terminal methionine residue, etc.

Also provided by the invention are chemically modified derivatives of the polypeptides of the invention which may provide additional advantages such as increased solubility, stability and circulating time of the polypeptide, or decreased immunogenicity (see U.S. Pat. No: 4,179,337). The chemical moieties for derivitization may be selected from water soluble polymers such as polyethylene glycol, ethylene glycol/propylene glycol copolymers, carboxymethylcellulose, dextran, polyvinyl alcohol and the like. The polypeptides may be modified at random positions within the molecule, or at predetermined positions within the molecule and may include one, two, three or more attached chemical moieties.

The invention further encompasses chemical derivitization of the polypeptides of the present invention, preferably where the chemical is a hydrophilic polymer residue. Exemplary hydrophilic polymers, including derivatives, may be those that include polymers in which the repeating units contain one or more hydroxy groups (polyhydroxy polymers), including, for example, poly(vinyl alcohol); polymers in which the repeating units contain one or more amino groups (polyamine polymers), including, for example, peptides, polypeptides, proteins and lipoproteins, such as albumin and natural lipoproteins; polymers in which the repeating units contain one or more carboxy groups (polycarboxy polymers), including, for example, carboxymethylcellulose, alginic acid and salts thereof, such as sodium and calcium alginate, glycosaminoglycans and salts thereof, including salts of hyaluronic acid, phosphorylated and sulfonated derivatives of carbohydrates, genetic material, such as interleukin-2 and interferon, and phosphorothioate oligomers; and polymers in which the repeating units contain one or more saccharide moieties (polysaccharide polymers), including, for example, carbohydrates.

The molecular weight of the hydrophilic polymers may vary, and is generally about 50 to about 5,000,000, with polymers having a molecular weight of about 100 to about 50,000 being preferred. The polymers may be branched or unbranched. More preferred polymers have a molecular weight of about 150 to about 10,000, with molecular weights of 200 to about 8,000 being even more preferred.

For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog).

Additional preferred polymers which may be used to derivatize polypeptides of the invention, include, for example, poly(ethylene glycol) (PEG), poly(vinylpyrrolidine), polyoxomers, polysorbate and poly(vinyl alcohol), with PEG polymers being particularly preferred. Preferred among the PEG polymers are PEG polymers having a molecular weight of from about 100 to about 10,000. More preferably, the PEG polymers have a molecular weight of from about 200 to about 8,000, with PEG 2,000, PEG 5,000 and PEG 8,000, which have molecular weights of 2,000, 5,000 and 8,000, respectively, being even more preferred. Other suitable hydrophilic polymers, in addition to those exemplified above, will be readily apparent to one skilled in the art based on the present disclosure. Generally, the polymers used may include polymers that can be attached to the polypeptides of the invention via alkylation or acylation reactions.

The polyethylene glycol molecules (or other chemical moieties) should be attached to the protein with consideration of effects on functional or antigenic domains of the protein. There are a number of attachment methods available to those skilled in the art, e.g., EP 0 401 384, herein incorporated by reference (coupling PEG to G-CSF), see also Malik et al., Exp. Hematol. 20:1028-1035 (1992) (reporting pegylation of GM-CSF using tresyl chloride). For example, polyethylene glycol may be covalently bound through amino acid residues via a reactive group, such as, a free amino or carboxyl-group. Reactive groups are those to which an activated polyethylene glycol molecule may be bound. The amino acid residues having a free amino group may include lysine residues and the N-terminal amino acid residues; those having a free carboxyl group may include aspartic acid residues glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group.

One may specifically desire proteins chemically modified at the N-terminus. Using polyethylene glycol as an illustration of the present composition, one may select from a variety of polyethylene glycol molecules (by molecular weight, branching, etc.), the proportion of polyethylene glycol molecules to protein (polypeptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective proteins chemically modified at the N-terminus modification may be accomplished by reductive alkylation which exploits differential reactivity of different types of primary amino groups (lysine versus the N-terminus) available for derivatization in a particular protein. Under the appropriate reaction conditions, substantially selective derivatization of the protein at the N-terminus with a carbonyl group containing polymer is achieved.

As with the various polymers exemplified above, it is contemplated that the polymeric residues may contain functional groups in addition, for example, to those typically involved in linking the polymeric residues to the polypeptides of the present invention. Such functionalities include, for example, carboxyl, amine, hydroxy and thiol groups. These functional groups on the polymeric residues can be further reacted, if desired, with materials that are generally reactive with such functional groups and which can assist in targeting specific tissues in the body including, for example, diseased tissue. Exemplary materials which can be reacted with the additional functional groups include, for example, proteins, including antibodies, carbohydrates, peptides, glycopeptides, glycolipids, lectins, and nucleosides.

In addition to residues of hydrophilic polymers, the chemical used to derivatize the polypeptides of the present invention can be a saccharide residue. Exemplary saccharides which can be derived include, for example, monosaccharides or sugar alcohols, such as erythrose, threose, ribose, arabinose, xylose, lyxose, fructose, sorbitol, mannitol and sedoheptulose, with preferred monosaccharides being fructose, mannose, xylose, arabinose, mannitol and sorbitol; and disaccharides, such as lactose, sucrose, maltose and cellobiose. Other saccharides include, for example, inositol and ganglioside head groups. Other suitable saccharides, in addition to those exemplified above, will be readily apparent to one skilled in the art based on the present disclosure. Generally, saccharides which may be used for derivitization include saccharides that can be attached to the polypeptides of the invention via alkylation or acylation reactions.

Moreover, the invention also encompasses derivitization of the polypeptides of the present invention, for example, with lipids (including cationic, anionic, polymerized, charged, synthetic, saturated, unsaturated, and any combination of the above, etc.). stabilizing agents.

The invention encompasses derivitization of the polypeptides of the present invention, for example, with compounds that may serve a stabilizing function (e.g., to increase the polypeptides half-life in solution, to make the polypeptides more water soluble, to increase the polypeptides hydrophilic or hydrophobic character, etc.). Polymers useful as stabilizing materials may be of natural, semi-synthetic (modified natural) or synthetic origin. Exemplary natural polymers include naturally occurring polysaccharides, such as, for example, arabinans, fructans, fucans, galactans, galacturonans, glucans, mannans, xylans (such as, for example, inulin), levan, fucoidan, carrageenan, galatocarolose, pectic acid, pectins, including amylose, pullulan, glycogen, amylopectin, cellulose, dextran, dextrin, dextrose, glucose, polyglucose, polydextrose, pustulan, chitin, agarose, keratin, chondroitin, dermatan, hyaluronic acid, alginic acid, xanthin gum, starch and various other natural homopolymer or heteropolymers, such as those containing one or more of the following aldoses, ketoses, acids or amines: erythose, threose, ribose, arabinose, xylose, lyxose, allose, altrose, glucose, dextrose, mannose, gulose, idose, galactose, talose, erythrulose, ribulose, xylulose, psicose, fructose, sorbose, tagatose, mannitol, sorbitol, lactose, sucrose, trehalose, maltose, cellobiose, glycine, serine, threonine, cysteine, tyrosine, asparagine, glutamine, aspartic acid, glutamic acid, lysine, arginine, histidine, glucuronic acid, gluconic acid, glucaric acid, galacturonic acid, mannuronic acid, glucosamine, galactosamine, and neuraminic acid, and naturally occurring derivatives thereof Accordingly, suitable polymers include, for example, proteins, such as albumin, polyalginates, and polylactide-coglycolide polymers. Exemplary semi-synthetic polymers include carboxymethylcellulose, hydroxymethylcellulose, hydroxypropylmethylcellulose, methylcellulose, and methoxycellulose. Exemplary synthetic polymers include polyphosphazenes, hydroxyapatites, fluoroapatite polymers, polyethylenes (such as, for example, polyethylene glycol (including for example, the class of compounds referred to as Pluronics.RTM., commercially available from BASF, Parsippany, N.J.), polyoxyethylene, and polyethylene terephthlate), polypropylenes (such as, for example, polypropylene glycol), polyurethanes (such as, for example, polyvinyl alcohol (PVA), polyvinyl chloride and polyvinylpyrrolidone), polyamides including nylon, polystyrene, polylactic acids, fluorinated hydrocarbon polymers, fluorinated carbon polymers (such as, for example, polytetrafluoroethylene), acrylate, methacrylate, and polymethylmethacrylate, and derivatives thereof. Methods for the preparation of derivatized polypeptides of the invention which employ polymers as stabilizing compounds will be readily apparent to one skilled in the art, in view of the present disclosure, when coupled with information known in the art, such as that described and referred to in Unger, U.S. Pat. No. 5,205,290, the disclosure of which is hereby incorporated by reference herein in its entirety.

Moreover, the invention encompasses additional modifications of the polypeptides of the present invention. Such additional modifications are known in the art, and are specifically provided, in addition to methods of derivitization, etc., in U.S. Pat. No. 6,028,066, which is hereby incorporated in its entirety herein.

The polypeptides of the invention may be in monomers or multimers (i.e., dimers, trimers, tetramers and higher multimers). Accordingly, the present invention relates to monomers and multimers of the polypeptides of the invention, their preparation, and compositions (preferably, Therapeutics) containing them. In specific embodiments, the polypeptides of the invention are monomers, dimers, trimers or tetramers. In additional embodiments, the multimers of the invention are at least dimers, at least trimers, or at least tetramers.

Multimers encompassed by the invention may be homomers or heteromers. As used herein, the term homomer, refers to a multimer containing only polypeptides corresponding to the amino acid sequence of SEQ ID NO:6, 8, 10, 12, 14, 16, 18, 20, 22, 24, 26, 28, 30, 32, 34, 36, 38, 40, 42, 44, 46, 48, 50, and/or 604 (including fragments, variants, splice variants, and fusion proteins, corresponding to these polypeptides as described herein). These homomers may contain polypeptides having identical or different amino acid sequences. In a specific embodiment, a homomer of the invention is a multimer containing only polypeptides having an identical amino acid sequence. In another specific embodiment, a homomer of the invention is a multimer containing polypeptides having different amino acid sequences. In specific embodiments, the multimer of the invention is a homodimer (e.g., containing polypeptides having identical or different amino acid sequences) or a homotrimer (e.g., containing polypeptides having identical and/or different amino acid sequences). In additional embodiments, the homomeric multimer of the invention is at least a homodimer, at least a homotrimer, or at least a homotetramer.

As used herein, the term heteromer refers to a multimer containing one or more heterologous polypeptides (i.e., polypeptides of different proteins) in addition to the polypeptides of the invention. In a specific embodiment, the multimer of the invention is a heterodimer, a heterotrimer, or a heterotetramer. In additional embodiments, the heteromeric multimer of the invention is at least a heterodimer, at least a heterotrimer, or at least a heterotetramer.

Multimers of the invention may be the result of hydrophobic, hydrophilic, ionic and/or covalent associations and/or may be indirectly linked, by for example, liposome formation. Thus, in one embodiment, multimers of the invention, such as, for example, homodimers or homotrimers, are formed when polypeptides of the invention contact one another in solution. In another embodiment, heteromultimers of the invention, such as, for example, heterotrimers or heterotetramers, are formed when polypeptides of the invention contact antibodies to the polypeptides of the invention (including antibodies to the heterologous polypeptide sequence in a fusion protein of the invention) in solution. In other embodiments, multimers of the invention are formed by covalent associations with and/or between the polypeptides of the invention. Such covalent associations may involve one or more amino acid residues contained in the polypeptide sequence (e.g., that recited in the sequence listing). In one instance, the covalent associations are cross-linking between cysteine residues located within the polypeptide sequences which interact in the native (i.e., naturally occurring) polypeptide. In another instance, the covalent associations are the consequence of chemical or recombinant manipulation. Alternatively, such covalent associations may involve one or more amino acid residues contained in the heterologous polypeptide sequence in a fusion protein of the invention.

In one example, covalent associations are between the heterologous sequence contained in a fusion protein of the invention (see, e.g., U.S. Pat. No. 5,478,925). In a specific example, the covalent associations are between the heterologous sequence contained in an Fc fusion protein of the invention (as described herein). In another specific example, covalent associations of fusion proteins of the invention are between heterologous polypeptide sequence from another protein that is capable of forming covalently associated multimers, such as for example, osteoprotegerin (see, e.g., International Publication NO: WO 98/49305, the contents of which are herein incorporated by reference in its entirety). In another embodiment, two or more polypeptides of the invention are joined through peptide linkers. Examples include those peptide linkers described in U.S. Pat. No. 5,073,627 (hereby incorporated by reference). Proteins comprising multiple polypeptides of the invention separated by peptide linkers may be produced using conventional recombinant DNA technology.

Another method for preparing multimer polypeptides of the invention involves use of polypeptides of the invention fused to a leucine zipper or isoleucine zipper polypeptide sequence. Leucine zipper and isoleucine zipper domains are polypeptides that promote multimerization of the proteins in which they are found. Leucine zippers were originally identified in several DNA-binding proteins (Landschulz et al., Science 240:1759, (1988)), and have since been found in a variety of different proteins. Among the known leucine zippers are naturally occurring peptides and derivatives thereof that dimerize or trimerize. Examples of leucine zipper domains suitable for producing soluble multimeric proteins of the invention are those described in PCT application WO 94/10308, hereby incorporated by reference. Recombinant fusion proteins comprising a polypeptide of the invention fused to a polypeptide sequence that dimerizes or trimerizes in solution are expressed in suitable host cells, and the resulting soluble multimeric fusion protein is recovered from the culture supernatant using techniques known in the art.

Trimeric polypeptides of the invention may offer the advantage of enhanced biological activity. Preferred leucine zipper moieties and isoleucine moieties are those that preferentially form trimers. One example is a leucine zipper derived from lung surfactant protein D (SPD), as described in Hoppe et al. (FEBS Letters 344:191, (1994)) and in U.S. patent application Ser. No. 08/446,922, hereby incorporated by reference. Other peptides derived from naturally occurring trimeric proteins may be employed in preparing trimeric polypeptides of the invention.

In another example, proteins of the invention are associated by interactions between Flag® polypeptide sequence contained in fusion proteins of the invention containing Flag® polypeptide sequence. In a further embodiment, associations proteins of the invention are associated by interactions between heterologous polypeptide sequence contained in Flag® fusion proteins of the invention and anti-Flag® antibody.

The multimers of the invention may be generated using chemical techniques known in the art. For example, polypeptides desired to be contained in the multimers of the invention may be chemically cross-linked using linker molecules and linker molecule length optimization techniques known in the art (see, e.g., U.S. Pat. No. 5,478,925, which is herein incorporated by reference in its entirety). Additionally, multimers of the invention may be generated using techniques known in the art to form one or more inter-molecule cross-links between the cysteine residues located within the sequence of the polypeptides desired to be contained in the multimer (see, e.g., U.S. Pat. No. 5,478,925, which is herein incorporated by reference in its entirety). Further, polypeptides of the invention may be routinely modified by the addition of cysteine or biotin to the C terminus or N-terminus of the polypeptide and techniques known in the art may be applied to generate multimers containing one or more of these modified polypeptides (see, e.g., U.S. Pat. No. 5,478,925, which is herein incorporated by reference in its entirety). Additionally, techniques known in the art may be applied to generate liposomes containing the polypeptide components desired to be contained in the multimer of the invention (see, e.g., U.S. Pat. No. 5,478,925, which is herein incorporated by reference in its entirety).

Alternatively, multimers of the invention may be generated using genetic engineering techniques known in the art. In one embodiment, polypeptides contained in multimers of the invention are produced recombinantly using fusion protein technology described herein or otherwise known in the art (see, e.g., U.S. Pat. No. 5,478,925, which is herein incorporated by reference in its entirety). In a specific embodiment, polynucleotides coding for a homodimer of the invention are generated by ligating a polynucleotide sequence encoding a polypeptide of the invention to a sequence encoding a linker polypeptide and then further to a synthetic polynucleotide encoding the translated product of the polypeptide in the reverse orientation from the original C-terminus to the N-terminus (lacking the leader sequence) (see, e.g., U.S. Pat. No. 5,478,925, which is herein incorporated by reference in its entirety). In another embodiment, recombinant techniques described herein or otherwise known in the art are applied to generate recombinant polypeptides of the invention which contain a transmembrane domain (or hydrophobic or signal peptide) and which can be incorporated by membrane reconstitution techniques into liposomes (see, e.g., U.S. Pat. No. 5,478,925, which is herein incorporated by reference in its entirety).

In addition, the polynucleotide insert of the present invention could be operatively linked to “artificial” or chimeric promoters and transcription factors. Specifically, the artificial promoter could comprise, or alternatively consist, of any combination of cis-acting DNA sequence elements that are recognized by trans-acting transcription factors. Preferably, the cis acting DNA sequence elements and trans-acting transcription factors are operable in mammals. Further, the trans-acting transcription factors of such “artificial” promoters could also be “artificial” or chimeric in design themselves and could act as activators or repressors to said “artificial” promoter.

### Methods of Use of The Allelic Polynucleotides and Polypeptides of the Present Invention

The determination of the polymorphic form(s) present in an individual at one or more polymorphic sites defined herein can be used in a number of methods.

In preferred embodiments, the polynucleotides and polypeptides of the present invention, including allelic and variant forms thereof, have uses which include, but are not limited to diagnosing individuals to identify whether a given individual has increased susceptibility or risk for liver disease, high cholesterol levels, myocardial infarction, resistance to statin drugs, particularly pravastatin, etc.

In another embodiment, the polynucleotides and polypeptides of the present invention, including allelic and variant forms thereof, either alone,or in combination with other polymorphic polynucleotides (haplotypes) are useful as genetic markers.

In preferred embodiments, the polynucleotides and polypeptides of the present invention, including allelic and variant forms thereof, have uses which include, but are not limited to diagnosing individuals to identify whether a given individual has increased susceptibility or risk for other conditions such as high cholesterol levels, and myocardial infarction using the genotype assays of the present invention, and diagnosing individuals to identify whether a given individual, upon administration of a statin, preferably pravastatin, and/or any other statin known in the art or described herein, has increased susceptibility or risk for high cholesterol levels, or myocardial infarction using the genotype assays of the present invention.

The polynucleotides and polypeptides of the present invention, including allelic and/or variant forms thereof, are useful for creating recombinant vectors and hosts cells for the expression of variant forms of the polypeptides of the present invention.

The polynucleotides and polypeptides of the present invention, including allelic and/or variant forms thereof, are useful for creating antagonists directed against these polynucleotides and polypeptides, particularly antibody antagonists, for diagnostic, and/or therapeutic applications.

Additionally, the polynucleotides and polypeptides of the present invention, including allelic and/or variant forms thereof, are useful for creating additional antagonists directed against these polynucleotides and polypeptides, which include, but are not limited to the design of antisense RNA, ribozymes, PNAs, recombinant zinc finger proteins (Wolfe, S A., Ramm, E I., Pabo, C O, Structure, Fold, Des., 8(7):739-50, (2000); Kang, J S., Kim, J S, J. Biol, Chem., 275(12):8742-8, (2000); Wang, B S., Pabo, C O, Proc, Natl, Acad, Sci, U.S.A., 96(17):9568-73, (1999); McColl, D J., Honchell, C D., Frankel, A D, Proc, Natl, Acad, Sci, U.S.A., 96(17):9521-6, (1999); Segal, D J., Dreier, B., Beerli, R R., Barbas, CF-3rd, Proc, Natl, Acad, Sci, U.S.A., 96(6):2758-63, (1999); Wolfe, S A., Greisman, H A., Ramm, E I., Pabo, C O, J. Mol, Biol., 285(5):1917-34, (1999); Pomerantz, J L., Wolfe, S A., Pabo, C O, Biochemistry., 37(4):965-70, (1998); Leon, O., Roth, M., Biol. Res. 33(1):21-30 (2000); Berg, J M., Godwin, H A, Ann. Rev. Biophys. Biomol. Struct., 26:357-71 (1997)), in addition to other types of antagonists which are either described elsewhere herein, or known in the art.

The polynucleotides and polypeptides of the present invention, including allelic and/or variant forms thereof, are useful for creating small molecule antagonists directed against the variant forms of these polynucleotides and polypeptides, preferably wherein such small molecules are useful as therapeutic and/or pharmaceutical compounds for the treatment, detection, prognosis, and/or prevention of the following, nonlimiting diseases and/or disorders, metabolic dieases, cardiovascular diseases, inflammatory diseases, high cholesterol, hypertension, and congestive heart failure.

The polynucleotides and polypeptides of the present invention, including allelic and/or variant forms thereof, are useful for the treatment of high cholesterol, myocardial infarction, hypertension, congestive heart failure, in addition to other diseases and/or conditions referenced elsewhere herein, through the application of gene therapy based regimens.

Agonists and/or antagonists of the polynucleotides and polypeptides of the present invention, including allelic and/or variant forms thereof, are useful for increasing or decreasing the flow of compounds transported by an organic anion transporter.

Agonists and/or antagonists of the polynucleotides and polypeptides of the present invention, including allelic and/or variant forms thereof, are useful for increasing or decreasing the flow of compounds transported by an organic anion transporter in the liver.

Agonists and/or antagonists of the polynucleotides and polypeptides of the present invention, including allelic and/or variant forms thereof, are useful for treating liver disease.

The polynucleotides and polypeptides of the present invention, including allelic and/or variant forms thereof, are useful in assays for identification of organic anion transport positive and negative modulators (i.e., agonists and/or antagonists) and organic anion transport carriers. The term “positive modulator” as used herein refers to an agent or compound that increases the rate or amount of transport of a compound into an organ, e.g., the liver, or an agent or compound that decreases the rate or amount of transport of a compound into an organ. The term “negative modulator” refers to a compound that is joined to a second compound to prevent the second compounds transport into or out of cells. The term “carrier” as used herein refers to an agent or compound that is transported by an OATP of the present invention and that is capable of being joined to or associated with another compound to chaperone that other compound into an organ, e.g., the liver. A carrier includes an agent that is used to transport a compound into an organ that is otherwise not transported into said organ, and includes an agent that increases the transport of a compound into an organ that is capable of being transported by an OATP.

The polynucleotides and polypeptides of the present invention, including allelic and/or variant forms thereof, are useful for treating coronary heart disease (CHD), identifying individuals at risk of CHD, and modulating low-density lipoprotein cholesterol (LDL-C).

Antagonists of the polynucleotides and polypeptides of the present invention are useful for patients that have been administered an anti-cancer, or anti-tumor, regimen, as such antagonists may increase the efficacy of such a regimen by diminishing the level of anti-cancer, or anti-tumor drug that is transported out of the cancer or tumor cells.

The polynucleotides and polypeptides of the present invention, including allelic and/or variant forms thereof, are useful detecting and/or predicting an individual's response to pravastatin treatment for LDL-cholesterol reduction, the an individual's response to pravastatin treatment for coronary artery disease prevention, an individual's response to HMG-CoA reductase inhibitors for LDL-cholesterol reduction, an individual's response to HMG-CoA reductase inhibitors for coronary artery disease prevention, an individual's response to drugs that use OATP2 for hepatic or cellular uptake, the prediction of the toxic effect of chemical compounds that use OATP2 for hepatic or cellular uptake, and/or an individual's response to xenobiotics that use OATP2 for hepatic or cellular uptake.

Prediction of pathogenesis of conditions mediated through endogenous compounds that use OATP2 for hepatic or cellular uptake, including but not limited to taurocholate, estrone sulfate, estradiol 17-D-glucuronide, leukotriene C4, prostaglandin E2, and thyroid hormone.

The polynucleotides and polypeptides of the present invention, including allelic and/or variant forms thereof, are useful for detecting and/or predicting the extent of hepatic statin uptake, preferably pravastatin, in dyslipidemic patients.

The polynucleotides and polypeptides of the present invention, including allelic and/or variant forms thereof, are useful for detecting and/or predicting LDL (low-density lipoprotein) and TG (tri-glyceride) lowering, and/or HDL (high-density lipoprotein) elevation in response to statin treatment, preferably pravastatin, in dyslipidemic patients.

The polynucleotides and polypeptides of the present invention, including allelic and/or variant forms thereof, are useful for detecting and/or predicting drug-drug interactions between two drugs that utilize OATP2 for uptake into hepatocytes. For example, polynucleotides and polypeptides of the present invention, including allelic and/or variant forms thereof, are useful for detecting and/or predicting drug-drug interactions between pravastatin, lovastatin, cerivastatin, simvastatin, pitivastatin, atorvastatin or rousuvastatin, and any other drug transported by OATP2.

The polynucleotides and polypeptides of the present invention, including allelic and/or variant forms thereof, are useful for detecting and/or predicting drug-endogenous substrate interactions between a drug and endogenous substance both of whom utilize OATP2 for uptake into hepatocytes. For example, polynucleotides and polypeptides of the present invention, including allelic and/or variant forms thereof, are useful for detecting and/or predicting drug-endogenous substrate interactions statins, which include, but are not limited to, pravastatin, lovastatin, cerivastatin, simvastatin, pitivastatin, atorvastatin or rousuvastatin, and any other endogenous substrate of OATP2 including cholate, taurocholate, thyroid hormones T3 and T4, DHEAS, estradiol-17beta-glucuronide, estrone-3-sulfate, prostaglandin E2, thromboxane B2, leukotriene C4, leukotriene E4, and bilirubin and its conjugates.

The polynucleotides and polypeptides of the present invention, including allelic and/or variant forms thereof, are useful for detecting and/or predicting the extent of hepatic statin clearance in dyslipidemic patients.

The polynucleotides and polypeptides of the present invention, including allelic and/or variant forms thereof, are useful for detecting and/or predicting drug-drug interactions between two drugs that utilize cMOAT for clearance out of the liver into the bile. For example, the polynucleotides and polypeptides of the present invention, including allelic and/or variant forms thereof, are useful for detecting and/or predicting drug interactions between pravastatin, lovastatin, cerivastatin, simvastatin, pitivastatin, atorvastatin or rousuvastatin, and any other drug transported by cMOAT.

The polynucleotides and polypeptides of the present invention, including allelic and/or variant forms thereof, are useful for detecting and/or predicting drug-endogenous substrate interactions between a drug and endogenous substance both of whom utilize cMOAT for clearance out of the liver into the bile. For example, the polynucleotides and polypeptides of the present invention, including allelic and/or variant forms thereof, are useful for detecting and/or predicting drug interactions between statins, which include, but are not limited to, pravastatin, lovastatin, cerivastatin, simvastatin, pitivastatin, atorvastatin or rousuvastatin, and any other endogenous substrate of OATP2 including cholate, taurocholate, thyroid hormones T3 and T4, DHEAS, estradiol-17beta-glucuronide, estrone-3-sulfate, prostaglandin E2, thromboxane B2, leukotriene C4, leukotriene E4, and bilirubin and its conjugates.

The polynucleotides and polypeptides of the present invention, including allelic and/or variant forms thereof, are useful for detecting and/or predicting the extent of removal of anti-tumor cytotoxic drugs from tumor cells (i.e. drug resistance) that express cMOAT in cancer patients during chemotherapy. For example, the polynucleotides and polypeptides of the present invention, including allelic and/or variant forms thereof, are useful for detecting and/or predicting the extent of removal of anti-tumor cytotoxic drugs from tumor cells, which include, for example, the following, non-limiting cytotoxic drugs including methotrexate, doxirubicin, cisplatin, CPT-11, SN-38, vincristine, and etoposide, in addition to any other cytotoxic drugs that may be transported by cMOAT and/or OATP2.

The polynucleotides and polypeptides of the present invention, including allelic and/or variant forms thereof, are useful for detecting and/or predicting response of cancer patients to anti-tumor therapy with drugs that are removed from tumor cells by cMOAT. For example, the polynucleotides and polypeptides of the present invention, including allelic and/or variant forms thereof, are useful for detecting and/or predicting the response of cancer patients to anti-tumor therapy with drugs that are removed from tumor cells by cMOAT, which include, for example, the following, non-limiting anti-tumor drugs including methotrexate, doxirubicin, cisplatin, CPT-11, SN-38, vincristine, and etoposide, in addition to the response by a patient to any other cytotoxic drugs that may be transported by cMOAT and/or OATP2.

Additional uses of the polynucleotides and polypeptides of the present invention are provided herein.

### A. Forensics

Determination of which polymorphic forms occupy a set of polymorphic sites in an individual identifies a set of polymorphic forms that distinguishes the individual. See generally National Research Council, The Evaluation of Forensic DNA Evidence (Eds. Pollard et al., National Academy Press, DC, 1996). The more sites that are analyzed, the lower the probability that the set of polymorphic forms in one individual is the same as that in an unrelated individual. Preferably, if multiple sites are analyzed, the sites are unlinked. Thus, polymorphisms of the invention are often used in conjunction with polymorphisms in distal genes. Preferred polymorphisms for use in forensics are biallelic because the population frequencies of two polymorphic forms can usually be determined with greater accuracy than those of multiple polymorphic forms at multi-allelic loci.

The capacity to identify a distinguishing or unique set of forensic markers in an individual is useful for forensic analysis. For example, one can determine whether a blood sample from a suspect matches a blood or other tissue sample from a crime scene by determining whether the set of polymorphic forms occupying selected polymorphic sites is the same in the suspect and the sample. If the set of polymorphic markers does not match between a suspect and a sample, it can be concluded (barring experimental eITor) that the suspect was not the source of the sample. If the set of markers does match, one can conclude that the DNA from the suspect is consistent with that found at the crime scene. If frequencies of the polymorphic forms at the loci tested have been determined (e.g., by analysis of a suitable population of individuals), one can perform a statistical analysis to determine the probability that a match of suspect and crime scene sample would occur by chance.

p(ID) is the probability that two random individuals have the same polymorphic or allelic form at a given polymorphic site. In biallelic loci, four genotypes are possible: AA, AB, BA, and BB. If alleles A and B occur in a haploid genome of the organism with frequencies x and y, the probability of each genotype in a diploid organism is (see WO 95/12607):

Homozygote: p(AA)=x²

Homozygote: p(BB)=y²=(1−x)²

Single Heterozygote: p(AB)=p(BA)=xy=x(1−x)

Both Heterozygotes: p(AB+BA)=2xy=2x(1−x)

The probability of identity at one locus (i.e., the probability that two individuals, picked at random from a population will have identical polymorphic forms at a given locus) is given by the equation:

p(ID)=(x²)²+(2xy)²+(y²)².

These calculations can be extended for any number of polymorphic forms at a given locus. For example, the probability of identity p(m) for a 3-allele system where the alleles have the frequencies in the population of x, y and z, respectively, is equal to the sum of the squares of the genotype frequencies:

p(ID)=x⁴+(2xy)²+(2yz)²+(2xz)²+z⁴+y⁴

In a locus of n alleles, the appropriate binomial expansion is used to calculate p(ID) and p(exc).

The cumulative probability of identity (cum p(ID)) for each of multiple unlinked loci is determined by multiplying the probabilities provided by each locus.

cum p(ID)=p(ID1)p(ID2)p(ID3) . . . p(IDn)

The cumulative probability of non-identity for n loci (i.e. the probability that two random individuals will be different at lor more loci) is given by the equation:

cum p(non1D)=1−cum p(ID).

If several polymorphic loci are tested, the cumulative probability of non-identity for random individuals becomes very high (e.g., one billion to one). Such probabilities can be taken into account together with other evidence in determining the guilt or innocence of the suspect.

### B. Paternity Testing

The object of paternity testing is usually to determine whether a male is the father of a child. In most cases, the mother of the child is known and thus, the mother's contribution to the child's genotype can be traced. Paternity testing investigates whether the part of the child's genotype not attributable to the mother is consistent with that of the putative father. Paternity testing can be performed by analyzing sets of polymorphisms in the putative father and the child.

If the set of polymorphisms in the child attributable to the father does not match the set of polymorphisms of the putative father, it can be concluded, barring experimental error, that the putative father is not the real father.

If the set of polymorphisms in the child attributable to the father does match the set of polymorphisms of the putative father, a statistical calculation can be performed to determine the probability of coincidental match.

The probability of parentage exclusion (representing the probability that a random male will have a polymorphic form at a given polymorphic site that makes him incompatible as the father) is given by the equation (see WQ 95/12607):

p(exc)=xy(1−xy)

where x and y are the population frequencies of alleles A and B of a biallelic polymorphic site.

(At a triallelic site p(exc)=xy(1−xy)+yz(1−yz)+xz(1−xz)+3xyz(1−xyz))),

where x, y and z and the respective population frequencies of alleles A, B and C).

The probability of non-exclusion is

p(non−exc)=1−p(exc)

The cumulative probability ofnon-exclusion (representing the value obtained when n loci are used) is thus:

cum p(non−exc)=p(non−exc1)p(non−exc2)p(non−exc3) . . . p(non−excn)

The cumulative probability of exclusion for n loci (representing the probability that a random male will be excluded)

cum p(exc)=1−cum p(non−exc).

If several polymorphic loci are included in the analysis, the cumulative probability of exclusion of a random male is very high. This probability can be taken into account in assessing the liability of a putative father whose polymorphic marker set matches the child's polymorphic marker set attributable to his/her father.

### C. Correlation of Polymorphisms with Phenotypic Traits

The polymorphisms of the invention may contribute to the phenotype of an organism in different ways. Some polymorphisms occur within a protein coding sequence and contribute to phenotype by affecting protein structure. The effect may be neutral, beneficial or detrimental, or both beneficial and detrimental, depending on the circumstances. For example, a heterozygous sickle cell mutation confers resistance to malaria, but a homozygous sickle cell mutation is usually lethal. Other polymorphisms occur in noncoding regions but may exert phenotypic effects indirectly via influence on replication, transcription, and translation. A single polymorphism may affect more than one phenotypic trait. Likewise, a single phenotypic trait may be affected by polymorphisms in different genes. Further, some polymorphisms predispose an individual to a distinct mutation that is causally related to a certain phenotype.

Phenotypic traits include diseases that have known but hitherto unmapped genetic components (e.g., agammaglobulimenia, diabetes insipidus, Lesch-Nyhan syndrome, muscular dystrophy, Wiskott-Aldrich syndrome, Fabry's disease, familial hypercholesterolemia, polycystic kidney disease, hereditary spherocytosis, von Willebrand's disease, tuberous sclerosis, hereditary hemorrhagic telangiectasia, familial colonic polyposis, Ehlers-Danlos syndrome, osteogenesis imperfecta, and acute intermittent porphyria). Phenotypic traits also include symptoms of, or susceptibility to, multifactorial diseases of which a component is or may be genetic, such as autoimmune diseases, inflammation, cancer, diseases of the nervous system, and infection by pathogenic microorganisms. Some examples of autoimmune diseases include rheumatoid arthritis, multiple sclerosis, diabetes (insulin-dependent and non-independent), systemic lupus erythematosus and Graves disease. Some examples of cancers include cancers of the bladder, brain, breast, colon, esophagus, kidney, leukemia, liver, lung, oral cavity, ovary, pancreas, prostate, skin, stomach and uterus. Phenotypic traits also include characteristics such as longevity, appearance (e.g., baldness, obesity), strength, speed, endurance, fertility, and susceptibility or receptivity to particular drugs or therapeutic treatments.

The correlation of one or more polymorphisms with phenotypic traits can be facilitated by knowledge of the gene product of the wild type (reference) gene. The genes in which SNPs of the present invention have been identified are genes which have been previously sequenced and characterized in one of their allelic forms. Thus, the SNPs of the invention can be used to identify correlations between one or another allelic form of the gene with a disorder with which the gene is associated, thereby identifying causative or predictive allelic forms of the gene.

Correlation is performed for a population of individuals who have been tested for the presence or absence of a phenotypic trait of interest and for polymorphic markers sets. T o perform such analysis, the presence or absence of a set of polymorphisms (i.e. a polymorphic set) is detennined for a set of the individuals, some ofwhom exhibit a particular trait, and some ofwhich exhibit lack of the trait. The alleles of each polymorphism of the set are then reviewed to determine whether the presence or absence of a particular allele is associated with the trait of interest. Correlation can be performed by standard statistical methods such as a 1C-squared test and statistically significant correlations between polymorphic form(s) and phenotypic characteristics are noted. For example, it might be found that the presence of allele A1 at polymorphism A correlates with heart disease. As a further example, it might be found that the combined presence of allele A1 at polymorphism A and allele B 1 at polymorphism B correlates with increased milk production of a farm animal.

Such correlations can be exploited in several ways. In the case of a strong correlation between a set of one or more polymorphic forms and a disease for which treatment is available, detection of the polymorphic form set in a human or animal patient may justify immediate administration of treatment, or at least the institution of regular monitoring of the patient. Detection of a polymorphic form correlated with serious disease in a couple contemplating a family may also be valuable to the couple in their reproductive decisions. For example, the female partner might elect to undergo in vitro fertilization to avoid the possibility of transmitting such a polymorphism from her husband to her offspring. In the case of a weaker, but still statistically significant correlation between a polymorphic set and human disease, immediate therapeutic intervention or monitoring may not be justified. Nevertheless, the patient can be motivated to begin simple life-style changes (e.g., diet, exercise) that can be accomplished at little cost to the patient but confer potential benefits in reducing the risk of conditions to which the patient may have increased susceptibility by virtue of variant alleles. Identification of a polymorphic set in a patient correlated with enhanced receptiveness to one of several treatment regimes for a disease indicates that this treatment regime should be followed.

For animals and plants, correlations between characteristics and phenotype are useful for breeding for desired characteristics. For example, Beitz et al, U.S. Pat. No. 5,292,639 discuss use of bovine mitochondrial polymorphisms in a breeding program to improve milk production in cows. To evaluate the effect of mtDNA D-loop sequence polymorphism on milk production, each cow was assigned a value of 1 ifvariant or 0 if wildtype with respect to a prototypical mitochondrial DNA sequence at each of 10 locations considered. Each production trait was analyzed individually with the following animal model:

Yᵢⱼₖₚₙ=υ+YSᵢ+Pⱼ+Xₖ+β₁+ . . . β₁₇+PEₙ+aₙ+eₚ

where Yᵢⱼₖₚₙ is the milk, fat, fat percentage, SNF , SNF percentage, energy concentration, or lactation energy record; υ is an overall mean; YSᵢ is the effect common to all cows calving in year-season; Xₖ is the effect common to cows in either the high or average selection line; β₁ to β₁₇ are the binomial regressions of production record on mtDNA D- loop sequence polymorphisms; PEₙ is permanent environmental effect common to all records of cow n; aₙ is effect of animal n and is composed of the additive genetic contribution of sire and dam breeding values and a Mendelian sampling effect; and eₚ is a random residual. It was found that eleven of seventeen polymorphisms tested influenced at least one production trait. Bovines having the best polymorphic forms for milk production at these eleven loci are used as parents for breeding the next generation of the herd.

### D. Genetic Mapping of Phenotypic Traits

The previous section concerns identifying correlations between phenotypic traits and polymorphisms that directly or indirectly contribute to those traits. The present section describes identification of a physical linkage between a genetic locus associated with a trait of interest and polymorphic markers that are not associated with the trait, but are in physical proximity with the genetic locus responsible for the trait and cosegregate with it. Such analysis is useful for mapping a genetic locus associated with a phenotypic trait to a chromosomal position, and thereby cloning gene(s) responsible for the trait. See Lander et al., Proc. Natl. Acad. Sci. (USA) 83:7353-7357 (1986); Lander et al., Proc. Natl. Acad. Sci. (USA)84:2363-2367 (1987); Donis-Keller et al., Cell 51:319-337 (1987); Lander et al., Genetics 121:185-199 (1989)). Genese localized by linkage can be cloned by a process known as directional cloning. See Winwright, Med. J. Australia 159:170-174 (1993); Collins, Nature Genetics 1:3-6 (1992).

Linkage studies are typically performed on members of a family. Available mmbers of the family are characterized for the presence or absence of a phenotypic trait and for a set of polymorhic markers. The distribution of polymorphic markers in an informative meiosis is then analyzed to determine which polymorphic markers cosegregate with a phenotypic trait. See, e.g., Kerem et al., Science 245:1073-1080 (1989); Monaco et al., Nature 316:842 (1985); Yamoka et al., Neurology 40:222-226 (1990); Rossiter et al., FASEB Journal, 5:21-27 (1991).

Linkage is analyzed by calculation of LOD (log of the odds) values. A LOS value is the realtive likelihood of obtaining oberved segregation data for a marker and a genetic locus when the ewo are located at a recombination fraction θ, versus the situtation in which the two are not linked, and thus segregating independetly (Thompson & Thompson, Genetics in Medicine (5th ed, W.B. Saunders Company, Philadelphia, 1991); Strachan, “Mapping the human genome” in The Human Gneome (BIOS Scientic Publishers Ltd, Oxford), Chapter 4). A series of likelihoos ratios are calculated at various recombination fractions (θ), ranging from θ=0.0 (coincident loci) to θ=0.50 (unlinked). Thus, the likelihoos ata given value of θ is: probability of data if loci linked at θ to probability of data if loci are unlinked. The computed likelihoods are usually expressed as the log10 of this ratio (i.e., a LOD score). For example, a LOD score of 3 indicates 1000:1 odds against an apparent obsered linkage being a coincidence. The use of logarithms allos data collected from different familites to be combined by simple algorithm. Computer programs are available for the calculation of LOD scores for differing values of θ (e.g., LIPED, MLINK (Lathrop, Proc. Nat. Acad. Sci. (USA)81, 3443-3446 (1984)). For any particular lod score, a recombination fraction may be determined from mathematical tables. See Smith et al., lvlathematical tables for research workers in human genetics (Churchill, London, 1961); Smith, Ann. Hum. Genet. 32,127-150 (1968). The value of θ at which the lod score is the highest is considered to be the best estimate of the recombination fraction. Positive lod score values suggest that the two loci are linked, whereas negative values suggest that linkage is less likely (at that value of θ) than the possibility that the two loci are unlinked. By convention, a combined lod score of +3 or greater ( equivalent to greater than 1000: 1 odds in favor of linkage) is considered definitive evidence that two loci are linked. Similarly, by convention, a negative lod score of −2 or less is taken as definitive evidence against linkage of the two loci being compared. Negative linkage data are useful in excluding a chromosome or a segment thereof from consideration. The search focuses on the remaining non-excluded chromosomal locations.

### IV. Modified Polypeptides and Gene Sequences

The invention further provides variant forms of nucleic acids and corresponding proteins. The nucleic acids comprise one of the sequences described in Table I, IV, V, or the polynucleotides encoding the polypeptides described in Table VI, in which the polymorphic position is occupied by one of the alternative bases for that position. Some nucleic acids encode full-length variant forms of proteins. Variant genes can be expressed in an expression vector in which a variant gene is operably linked to a native or other promoter. Usually, the promoter is a eukaryotic promoter for expression in a mammalian cell. The transcription regulation sequences typically include a heterologous promoter and optionally an enhancer which is recognized by the host. The selection of an appropriate promoter, for example trp, lac, phage promoters, glycolytic enzyme promoters and tRNA promoters, depends on the host selected. Commercially available expression vectors can be used. Vectors can include host-recognized replication systems, amplifiable genes, selectable markers, host sequences useful for insertion into the host genome, and the like.

The means of introducing the expression construct into a host cell varies depending upon the particular construction and the target host. Suitable means include fusion, conjugation, transfection, transduction, electroporation or injection, as described in Sambrook, supra. A wide variety of host cells can be employed for expression of the variant gene, both prokaryotic and eukaryotic. Suitable host cells include bacteria such as E. coli, yeast, filamentous fungi, insect cells, mammalian cells, typically immortalized, e.g. , mouse, CHO, human and monkey cell lines and derivatives thereof. Preferred host cells are able to process the variant gene product to produce an appropriate mature polypeptide. Processing includes glycosylation, ubiquitination, disulfide bond formation, general post-translational modification, and the like. As used herein, “gene product” includes mRNA, peptide and protein products.

The protein may be isolated by conventional means of protein biochemistry and purification to obtain a substantially pure product, i.e., 80,95 or 99% free of cell component contaminants, as described in Jacoby, Methods in Enzymology Volume 104, Academic Press, New York (1984); Scopes, Protein Purification, Principles and Practice, 2nd Edition, Springer-Verlag, New York (1987); and Deutscher (ed), Guide to Protein Purification, Methods in Enzymology, Vol. 182 (1990). If the protein is secreted, it can be isolated from the supernatant in which the host cell is grown. If not secreted, the protein can be isolated from a lysate of the host cells.

The invention further provides transgenic nonhuman animals capable of expressing an exogenous variant gene and/or having one or both alleles of an endogenous variant gene inactivated. Expression of an exogenous variant gene is usually achieved by operably linking the gene to a promoter and optionally an enhancer, and microinjecting the construct into a zygote. See Hogan et al., “Manipulating the Mouse Embryo, A Laboratory Manual,” Cold Spring Harbor Laboratory Inactivation of endogenous variant genes can be achieved by forming a trans gene in which a cloned variant gene is inactivated by insertion ofa positive selection marker. See Capecchi, Science 244, 1288-1292 (1989). The trans gene is then introduced into an embryonic stem cell, where it undergoes homologous recombination with an endogenous variant gene. Mice and other rodents are preferred animals. Such animals provide useful drug screening systems.

In addition to substantially full-length polypeptides expressed by variant genes, the present invention includes biologically active fragments of the polypeptides, or analogs thereof, including organic molecules which simulate the interactions of the peptides. Biologically active fragments include any portion of the full-length polypeptide which confers a biological function on the variant gene product, including ligand binding, and antibody binding. Ligand binding includes binding by nucleic acids, proteins or polypeptides, small biologically active molecules, or large cellular structures.

Polyclonal and/or monoclonal antibodies that specifically bind to variant gene products but not to corresponding prototypical gene products are also provided. Antibodies can be made by injecting mice or other animals with the variant gene product or synthetic peptide fragments thereof. Monoclonal antibodies are screened as are described, for example, in Harlow & Lane, Antibodies, A Laboratory Manual, Cold Spring Harbor Press, New York (1988); Goding, Monoclonal antibodies, Principles and Practice (2d ed.) Academic Press, New York (1986). Monoclonal antibodies are tested for specific immunoreactivity with a variant gene product and lack of immunoreactivity to the corresponding prototypical gene product. These antibodies are useful in diagnostic assays for detection of the variant form, or as an active ingredient in a pharmaceutical composition.

### V. Haplotype Based Genetic Analysis

The invention further provides methods of applying the polynucleotides and polypeptides of the present invention to the elucidation of haplotypes. Such haplotypes may be associated with any one or more of the disease conditions referenced elsewhere herein. A “haplotype” is defined as the pattern of a set of alleles of single nucleotide polymorphisms along a chromosome. For example, consider the case of three single nucleotide polymorphisms (SNP1, SNP2, and SNP3) in one chromosome region, of which SNP1 is an A/G polymorphism, SNP2 is a G/C polymorphism, and SNP3 is an A/C polymorphism. A and G are the alleles for the first, G and C for the second and A and C for the third SNP. Given two alleles for each SNP, there are three possible genotypes for individuals at each SNP. For example, for the first SNP, A/A, A/G and G/G are the possible genotypes for individuals. When an individual has a genotype for a SNP in which the alleles are not the same, for example A/G for the first SNP, then the individual is a heterozygote. When an individual has an A/G genotype at SNP1, G/C genotype at SNP2, and A/C genotype at SNP3 (FIG. 39), there are four possible combinations of haplotypes (A, B, C, and D) for this individual. The set of SNP genotypes of this individual alone would not provide sufficient information to resolve which combination of haplotypes this individual possesses. However, when this individual's parents' genotypes are available, haplotypes could then be assigned unambiguously. For example, if one parent had an A/A genotype at SNP1, a G/C genotype at SNP2, and an A/A genotype at SNP3, and the other parent had an A/G genotype at SNP1, C/C genotype at SNP2, and C/C genotype at SNP3, while the child was a heterozygote at all three SNPs (FIG. 40), there is only one possible haplotype combination, assuming there was no crossing over in this region during meiosis.

When the genotype information of relatives is not available, haplotype assignment can be done using the long range-PCR method (Clark, A. G. Mol Biol Evol 7(2): 111-22 (1990); Clark, A. G., K. M. Weiss, et al. Am J Hum Genet 63(2): 595-612 (1998); Fullerton, S. M., A. G. Clark, et al., Am J Hum. Genet 67(4): 881-900 (2000); Templeton, A. R., A. G. Clark, et al., Am J Hum Genet 66(1): 69-83 (2000)). When the genotyping result of the SNPs of interest are available from general population samples, the most likely haplotypes can also be assigned using statistical methods (Excoffier, L. and M. Slatkin. Mol Biol Evol 12(5): 921-7 (1995); Fallin, D. and N. J. Schork, Am J Hum Genet 67(4): 947-59 (2000); Long, J. C., R. C. Williams, et al., Am J Hum Genet 56(3): 799-810 (1995)).

Once an individual's haplotype in a certain chromosome region (i.e., locus) has been determined, it can be used as a tool for genetic association studies using different methods, which include, for example, haplotype relative risk analysis (Knapp, M., S. A. Seuchter, et al., Am J Hum Genet 52(6): 1085-93 (1993); Li, T., M. Arranz, et al., Schizophr Res 32(2): 87-92 (1998); Matise, T. C., Genet Epidemiol 12(6): 641-5 (1995); Ott, J., Genet Epidemiol 6(1): 127-30 (1989); Terwilliger, J. D. and J. Ott, Hum Hered 42(6): 337-46 (1992)). Haplotype based genetic analysis, using a combination of SNPs, provides increased detection sensitivity, and hence statistical significance, for genetic associations of diseases, as compared to analyses using individual SNPs as markers. Multiple SNPs present in a single gene or a continuous chromosomal region are useful for such haplotype-based analyses.

### VI. Kits

The invention further provides kits comprising at least one agent for identifying which alleleic form of the SNPs identified herein is present in a sample. For example, suitable kits can comprise at least one antibody specific for a particular protein or peptide encoded by one alleleic form of the gene, or allele-specific oligonucleotide as described herein. Often, the kits contain one or more pairs of allele-specific oligonucleotides hybridizing to different forms of a polymorphism. In some kits, the allele-specific oligonucleotides are provided immobilized to a substrate. For example, the same substrate can comprise allele-specific oligonucleotide probes for detecting at least 1, 10, 100 or all of the polymorphisms shown in Tables I, IV, V, or VI. Optional additional components of the kit include, for example, restriction enzymes, reverse-transcriptase or polymerase, the substrate nucleoside triphosphates, means used to label (for example, an avidin-enzyme conjugate and enzyme substrate and chromogen if the label is biotin), and the appropriate buffers for reverse transcription, PCR, or hybridization reactions. Usually, the kit also contains instructions for carrying out the methods.

### Uses of the Polynucleotides

Each of the polynucleotides identified herein can be used in numerous ways as reagents. The following description should be considered exemplary and utilizes known techniques.

The polynucleotides of the present invention are useful for chromosome identification. There exists an ongoing need to identify new chromosome markers, since few chromosome marking reagents, based on actual sequence data (repeat polymorphisms), are presently available. Each polynucleotide of the present invention can be used as a chromosome marker.

Briefly, sequences can be mapped to chromosomes by preparing PCR primers (preferably 15-25 bp) from the sequences shown in SEQ ID NO:5, 7, 9, 11, 13, 15, 17, 19, 21, 23, 25, 27, 29, 31, 33, 35, 37, 39, 41, 43, 45, 47, 49, and/or 603. Primers can be selected using computer analysis so that primers do not span more than one predicted exon in the genomic DNA. These primers are then used for PCR screening of somatic cell hybrids containing individual human chromosomes. Only those hybrids containing the human gene corresponding to the SEQ ID NO:5, 7, 9, 11, 13, 15, 17, 19, 21, 23, 25, 27, 29, 31, 33, 35, 37, 39, 41, 43, 45, 47, 49, and/or 603 will yield an amplified fragment.

Similarly, somatic hybrids provide a rapid method of PCR mapping the polynucleotides to particular chromosomes. Three or more clones can be assigned per day using a single thermal cycler. Moreover, sublocalization of the polynucleotides can be achieved with panels of specific chromosome fragments. Other gene mapping strategies that can be used include in situ hybridization, prescreening with labeled flow-sorted chromosomes, and preselection by hybridization to construct chromosome specific-cDNA libraries.

Precise chromosomal location of the polynucleotides can also be achieved using fluorescence in situ hybridization (FISH) of a metaphase chromosomal spread. This technique uses polynucleotides as short as 500 or 600 bases; however, polynucleotides 2,000-4,000 bp are preferred. For a review of this technique, see Verma et al., “Human Chromosomes: a Manual of Basic Techniques,” Pergamon Press, New York (1988).

For chromosome mapping, the polynucleotides can be used individually (to mark a single chromosome or a single site on that chromosome) or in panels (for marking multiple sites and/or multiple chromosomes). Preferred polynucleotides correspond to the noncoding regions of the cDNAs because the coding sequences are more likely conserved within gene families, thus increasing the chance of cross hybridization during chromosomal mapping.

Once a polynucleotide has been mapped to a precise chromosomal location, the physical position of the polynucleotide can be used in linkage analysis. Linkage analysis establishes coinheritance between a chromosomal location and presentation of a particular disease. Disease mapping data are known in the art. Assuming 1 megabase mapping resolution and one gene per 20 kb, a cDNA precisely localized to a chromosomal region associated with the disease could be one of 50-500 potential causative genes.

Thus, once coinheritance is established, differences in the polynucleotide and the corresponding gene between affected and unaffected organisms can be examined. First, visible structural alterations in the chromosomes, such as deletions or translocations, are examined in chromosome spreads or by PCR. If no structural alterations exist, the presence of point mutations are ascertained. Mutations observed in some or all affected organisms, but not in normal organisms, indicates that the mutation may cause the disease. However, complete sequencing of the polypeptide and the corresponding gene from several normal organisms is required to distinguish the mutation from a polymorphism. If a new polymorphism is identified, this polymorphic polypeptide can be used for further linkage analysis.

Furthermore, increased or decreased expression of the gene in affected organisms as compared to unaffected organisms can be assessed using polynucleotides of the present invention. Any of these alterations (altered expression, chromosomal rearrangement, or mutation) can be used as a diagnostic or prognostic marker.

Thus, the invention also provides a diagnostic method useful during diagnosis of a disorder, involving measuring the expression level of polynucleotides of the present invention in cells or body fluid from an organism and comparing the measured gene expression level with a standard level of polynucleotide expression level, whereby an increase or decrease in the gene expression level compared to the standard is indicative of a disorder.

By “measuring the expression level of a polynucleotide of the present invention” is intended qualitatively or quantitatively measuring or estimating the level of the polypeptide of the present invention or the level of the mRNA encoding the polypeptide in a first biological sample either directly (e.g., by determining or estimating absolute protein level or mRNA level) or relatively (e.g., by comparing to the polypeptide level or mRNA level in a second biological sample). Preferably, the polypeptide level or mRNA level in the first biological sample is measured or estimated and compared to a standard polypeptide level or mRNA level, the standard being taken from a second biological sample obtained from an individual not having the disorder or being determined by averaging levels from a population of organisms not having a disorder. As will be appreciated in the art, once a standard polypeptide level or mRNA level is known, it can be used repeatedly as a standard for comparison.

By “biological sample” is intended any biological sample obtained from an organism, body fluids, cell line, tissue culture, or other source which contains the polypeptide of the present invention or mRNA. As indicated, biological samples include body fluids (such as the following non-limiting examples, sputum, amniotic fluid, urine, saliva, breast milk, secretions, interstitial fluid, blood, serum, spinal fluid, etc.) which contain the polypeptide of the present invention, and other tissue sources found to express the polypeptide of the present invention. Methods for obtaining tissue biopsies and body fluids from organisms are well known in the art. Where the biological sample is to include mRNA, a tissue biopsy is the preferred source.

The method(s) provided above may Preferably be applied in a diagnostic method and/or kits in which polynucleotides and/or polypeptides are attached to a solid support. In one exemplary method, the support may be a “gene chip” or a “biological chip” as described in U.S. Pat. Nos. 5,837,832, 5,874,219, and 5,856,174. Further, such a gene chip with polynucleotides of the present invention attached may be used to identify polymorphisms between the polynucleotide sequences, with polynucleotides isolated from a test subject. The knowledge of such polymorphisms (i.e. their location, as well as, their existence) would be beneficial in identifying disease loci for many disorders, including proliferative diseases and conditions. Such a method is described in U.S. Pat. Nos. 5,858,659 and 5,856,104. The U.S. Patents referenced supra are hereby incorporated by reference in their entirety herein.

The present invention encompasses polynucleotides of the present invention that are chemically synthesized, or reproduced as peptide nucleic acids (PNA), or according to other methods known in the art. The use of PNAs would serve as the preferred form if the polynucleotides are incorporated onto a solid support, or gene chip. For the purposes of the present invention, a peptide nucleic acid (PNA) is a polyamide type of DNA analog and the monomeric units for adenine, guanine, thymine and cytosine are available commercially (Perceptive Biosystems). Certain components of DNA, such as phosphorus, phosphorus oxides, or deoxyribose derivatives, are not present in PNAs. As disclosed by P. E. Nielsen, M. Egholm, R. H. Berg and O. Buchardt, Science 254, 1497 (1991); and M. Egholm, O. Buchardt, L. Christensen, C. Behrens, S. M. Freier, D. A. Driver, R. H. Berg, S. K. Kim, B. Norden, and P. E. Nielsen, Nature 365, 666 (1993), PNAs bind specifically and tightly to complementary DNA strands and are not degraded by nucleases. In fact, PNA binds more strongly to DNA than DNA itself does. This is probably because there is no electrostatic repulsion between the two strands, and also the polyamide backbone is more flexible. Because of this, PNA/DNA duplexes bind under a wider range of stringency conditions than DNA/DNA duplexes, making it easier to perform multiplex hybridization. Smaller probes can be used than with DNA due to the stronger binding characteristics of PNA:DNA hybrids. In addition, it is more likely that single base mismatches can be determined with PNA/DNA hybridization because a single mismatch in a PNA/DNA 15-mer lowers the melting point (T.sub.m) by 8°-20° C., vs. 4°-16° C. for the DNA/DNA 15-mer duplex. Also, the absence of charge groups in PNA means that hybridization can be done at low ionic strengths and reduce possible interference by salt during the analysis.

In addition to the foregoing, a polynucleotide can be used to control gene expression through triple helix formation or antisense DNA or RNA. Antisense techniques are discussed, for example, in Okano, J. Neurochem. 56: 560 (1991); “Oligodeoxynucleotides as Antisense Inhibitors of Gene Expression, CRC Press, Boca Raton, FL (1988). Triple helix formation is discussed in, for instance Lee et al., Nucleic Acids Research 6: 3073 (1979); Cooney et al., Science 241: 456 (1988); and Dervan et al., Science 251: 1360 (1991). Both methods rely on binding of the polynucleotide to a complementary DNA or RNA. For these techniques, preferred polynucleotides are usually oligonucleotides 20 to 40 bases in length and complementary to either the region of the gene involved in transcription (triple helix—see Lee et al., Nucl. Acids Res. 6:3073 (1979); Cooney et al., Science 241:456 (1988); and Dervan et al., Science 251:1360 (1991) ) or to the mRNA itself (antisense—Okano, J. Neurochem. 56:560 (1991); Oligodeoxy-nucleotides as Antisense Inhibitors of Gene Expression, CRC Press, Boca Raton, FL (1988).) Triple helix formation optimally results in a shut-off of RNA transcription from DNA, while antisense RNA hybridization blocks translation of an mRNA molecule into polypeptide. Both techniques are effective in model systems, and the information disclosed herein can be used to design antisense or triple helix polynucleotides in an effort to treat or prevent disease.

The present invention encompasses the addition of a nuclear localization signal, operably linked to the 5′ end, 3′ end, or any location therein, to any of the oligonucleotides, antisense oligonucleotides, triple helix oligonucleotides, ribozymes, PNA oligonucleotides, and/or polynucleotides, of the present invention. See, for example, G. Cutrona, et al., Nat. Biotech., 18:300-303, (2000); which is hereby incorporated herein by reference.

Polynucleotides of the present invention are also useful in gene therapy. One goal of gene therapy is to insert a normal gene into an organism having a defective gene, in an effort to correct the genetic defect. The polynucleotides disclosed in the present invention offer a means of targeting such genetic defects in a highly accurate manner. Another goal is to insert a new gene that was not present in the host genome, thereby producing a new trait in the host cell. In one example, polynucleotide sequences of the present invention may be used to construct chimeric RNA/DNA oligonucleotides corresponding to said sequences, specifically designed to induce host cell mismatch repair mechanisms in an organism upon systemic injection, for example (Bartlett, R. J., et al., Nat. Biotech, 18:615-622 (2000), which is hereby incorporated by reference herein in its entirety). Such RNA/DNA oligonucleotides could be designed to correct genetic defects in certain host strains, and/or to introduce desired phenotypes in the host (e.g., introduction of a specific polymorphism within an endogenous gene corresponding to a polynucleotide of the present invention that may ameliorate and/or prevent a disease symptom and/or disorder, etc.). Alternatively, the polynucleotide sequence of the present invention may be used to construct duplex oligonucleotides corresponding to said sequence, specifically designed to correct genetic defects in certain host strains, and/or to introduce desired phenotypes into the host (e.g., introduction of a specific polymorphism within an endogenous gene corresponding to a polynucleotide of the present invention that may ameliorate and/or prevent a disease symptom and/or disorder, etc). Such methods of using duplex oligonucleotides are known in the art and are encompassed by the present invention (see EP1007712, which is hereby incorporated by reference herein in its entirety).

The polynucleotides are also useful for identifying organisms from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identifying personnel. This method does not suffer from the current limitations of “Dog Tags” which can be lost, switched, or stolen, making positive identification difficult. The polynucleotides of the present invention can be used as additional DNA markers for RFLP.

The polynucleotides of the present invention can also be used as an alternative to RFLP, by determining the actual base-by-base DNA sequence of selected portions of an organisms genome. These sequences can be used to prepare PCR primers for amplifying and isolating such selected DNA, which can then be sequenced. Using this technique, organisms can be identified because each organism will have a unique set of DNA sequences. Once an unique ID database is established for an organism, positive identification of that organism, living or dead, can be made from extremely small tissue samples. Similarly, polynucleotides of the present invention can be used as polymorphic markers, in addition to, the identification of transformed or non-transformed cells and/or tissues.

There is also a need for reagents capable of identifying the source of a particular tissue. Such need arises, for example, when presented with tissue of unknown origin. Appropriate reagents can comprise, for example, DNA probes or primers specific to particular tissue prepared from the sequences of the present invention. Panels of such reagents can identify tissue by species and/or by organ type. In a similar fashion, these reagents can be used to screen tissue cultures for contamination. Moreover, as mentioned above, such reagents can be used to screen and/or identify transformed and non-transformed cells and/or tissues.

In the very least, the polynucleotides of the present invention can be used as molecular weight markers on Southern gels, as diagnostic probes for the presence of a specific mRNA in a particular cell type, as a probe to “subtract-out” known sequences in the process of discovering novel polynucleotides, for selecting and making oligomers for attachment to a “gene chip” or other support, to raise anti-DNA antibodies using DNA immunization techniques, and as an antigen to elicit an immune response.

### Uses of the Polypeptides

Each of the polypeptides identified herein can be used in numerous ways. The following description should be considered exemplary and utilizes known techniques.

A polypeptide of the present invention can be used to assay protein levels in a biological sample using antibody-based techniques. For example, protein expression in tissues can be studied with classical immunohistological methods. (Jalkanen, M., et al., J. Cell. Biol. 101:976-985 (1985); Jalkanen, M., et al., J. Cell . Biol. 105:3087-3096 (1987).) Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes, such as iodine (125I, 121I), carbon (14C), sulfur (35S), tritium (3H), indium (112In), and technetium (99mTc), and fluorescent labels, such as fluorescein and rhodamine, and biotin.

In addition to assaying protein levels in a biological sample, proteins can also be detected in vivo by imaging. Antibody labels or markers for in vivo imaging of protein include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the antibody by labeling of nutrients for the relevant hybridoma.

A protein-specific antibody or antibody fragment which has been labeled with an appropriate detectable imaging moiety, such as a radioisotope (for example, 131I, 112In, 99mTc), a radio-opaque substance, or a material detectable by nuclear magnetic resonance, is introduced (for example, parenterally, subcutaneously, or intraperitoneally) into the mammal. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of 99 mTc. The labeled antibody or antibody fragment will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).) Thus, the invention provides a diagnostic method of a disorder, which involves (a) assaying the expression of a polypeptide of the present invention in cells or body fluid of an individual; (b) comparing the level of gene expression with a standard gene expression level, whereby an increase or decrease in the assayed polypeptide gene expression level compared to the standard expression level is indicative of a disorder. With respect to cancer, the presence of a relatively high amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Moreover, polypeptides of the present invention can be used to treat, prevent, and/or diagnose disease. For example, patients can be administered a polypeptide of the present invention in an effort to replace absent or decreased levels of the polypeptide (e.g., insulin), to supplement absent or decreased levels of a different polypeptide (e.g., hemoglobin S for hemoglobin B, SOD, catalase, DNA repair proteins), to inhibit the activity of a polypeptide (e.g., an oncogene or tumor suppressor), to activate the activity of a polypeptide (e.g., by binding to a receptor), to reduce the activity of a membrane bound receptor by competing with it for free ligand (e.g., soluble TNF receptors used in reducing inflammation), or to bring about a desired response (e.g., blood vessel growth inhibition, enhancement of the immune response to proliferative cells or tissues).

Similarly, antibodies directed to a polypeptide of the present invention can also be used to treat, prevent, and/or diagnose disease. For example, administration of an antibody directed to a polypeptide of the present invention can bind and reduce overproduction of the polypeptide. Similarly, administration of an antibody can activate the polypeptide, such as by binding to a polypeptide bound to a membrane (receptor).

At the very least, the polypeptides of the present invention can be used as molecular weight markers on SDS-PAGE gels or on molecular sieve gel filtration columns using methods well known to those of skill in the art. Polypeptides can also be used to raise antibodies, which in turn are used to measure protein expression from a recombinant cell, as a way of assessing transformation of the host cell. Moreover, the polypeptides of the present invention can be used to test the following biological activities.

### Gene Therapy Methods

Another aspect of the present invention is to gene therapy methods for treating or preventing disorders, diseases and conditions. The gene therapy methods relate to the introduction of nucleic acid (DNA, RNA and antisense DNA or RNA) sequences into an animal to achieve expression of a polypeptide of the present invention. This method requires a polynucleotide which codes for a polypeptide of the invention that operatively linked to a promoter and any other genetic elements necessary for the expression of the polypeptide by the target tissue. Such gene therapy and delivery techniques are known in the art, see, for example, WO90/11092, which is herein incorporated by reference.

Thus, for example, cells from a patient may be engineered with a polynucleotide (DNA or RNA) comprising a promoter operably linked to a polynucleotide of the invention ex vivo, with the engineered cells then being provided to a patient to be treated with the polypeptide. Such methods are well-known in the art. For example, see Belldegrun et al., J. Natl. Cancer Inst., 85:207-216 (1993); Ferrantini et al., Cancer Research, 53:107-1112 (1993); Ferrantini et al., J. Immunology 153: 4604-4615 (1994); Kaido, T., et al., Int. J. Cancer 60: 221-229 (1995); Ogura et al., Cancer Research 50: 5102-5106 (1990); Santodonato, et al., Human Gene Therapy 7:1-10 (1996); Santodonato, et al., Gene Therapy 4:1246-1255 (1997); and Zhang, et al., Cancer Gene Therapy 3: 31-38 (1996)), which are herein incorporated by reference. In one embodiment, the cells which are engineered are arterial cells. The arterial cells may be reintroduced into the patient through direct injection to the artery, the tissues surrounding the artery, or through catheter injection.

As discussed in more detail below, the polynucleotide constructs can be delivered by any method that delivers injectable materials to the cells of an animal, such as, injection into the interstitial space of tissues (heart, muscle, skin, lung, liver, and the like). The polynucleotide constructs may be delivered in a pharmaceutically acceptable liquid or aqueous carrier.

In one embodiment, the polynucleotide of the invention is delivered as a naked polynucleotide. The term “naked” polynucleotide, DNA or RNA refers to sequences that are free from any delivery vehicle that acts to assist, promote or facilitate entry into the cell, including viral sequences, viral particles, liposome formulations, lipofectin or precipitating agents and the like. However, the polynucleotides of the invention can also be delivered in liposome formulations and lipofectin formulations and the like can be prepared by methods well known to those skilled in the art. Such methods are described, for example, in U.S. Pat. Nos. 5,593,972, 5,589,466, and 5,580,859, which are herein incorporated by reference.

The polynucleotide vector constructs of the invention used in the gene therapy rnethod are preferably constructs that will not integrate into the host genome nor will they contain sequences that allow for replication. Appropriate vectors include pWLNEO, pSV2CAT, pOG44, pXT1 and pSG available from Stratagene; pSVK3, pBPV, pMSG and pSVL available from Pharmacia; and pEF1/V5, pcDNA3.1, and pRc/CMV2 available from Invitrogen. Other suitable vectors will be readily apparent to the skilled artisan.

Any strong promoter known to those skilled in the art can be used for driving the expression of polynucleotide sequence of the invention. Suitable promoters include adenoviral promoters, such as the adenoviral major late promoter; or heterologous promoters, such as the cytomegalovirus (CMV) promoter; the respiratory syncytial virus (RSV) promoter; inducible promoters, such as the MMT promoter, the metallothionein promoter; heat shock promoters; the albumin promoter; the ApoAI promoter; human globin promoters; viral thymidine kinase promoters, such as the Herpes Simplex thymidine kinase promoter; retroviral LTRs; the b-actin promoter; and human growth hormone promoters. The promoter also may be the native promoter for the polynucleotides of the invention.

Unlike other gene therapy techniques, one major advantage of introducing naked nucleic acid sequences into target cells is the transitory nature of the polynucleotide synthesis in the cells. Studies have shown that non-replicating DNA sequences can be introduced into cells to provide production of the desired polypeptide for periods of up to six months.

The polynucleotide construct of the invention can be delivered to the interstitial space of tissues within the an animal, including of muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue. Interstitial space of the tissues comprises the intercellular, fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels. Delivery to the interstitial space of muscle tissue is preferred for the reasons discussed below. They may be conveniently delivered by injection into the tissues comprising these cells. They are preferably delivered to and expressed in persistent, non-dividing cells which are differentiated, although delivery and expression may be achieved in non-differentiated or less completely differentiated cells, such as, for example, stem cells of blood or skin fibroblasts. In vivo muscle cells are particularly competent in their ability to take up and express polynucleotides.

For the naked nucleic acid sequence injection, an effective dosage amount of DNA or RNA will be in the range of from about 0.05 mg/kg body weight to about 50 mg/kg body weight. Preferably the dosage will be from about 0.005 mg/kg to about 20 mg/kg and more preferably from about 0.05 mg/kg to about 5 mg/kg. Of course, as the artisan of ordinary skill will appreciate, this dosage will vary according to the tissue site of injection. The appropriate and effective dosage of nucleic acid sequence can readily be determined by those of ordinary skill in the art and may depend on the condition being treated and the route of administration.

The preferred route of administration is by the parenteral route of injection into the interstitial space of tissues. However, other parenteral routes may also be used, such as, inhalation of an aerosol formulation particularly for delivery to lungs or bronchial tissues, throat or mucous membranes of the nose. In addition, naked DNA constructs can be delivered to arteries during angioplasty by the catheter used in the procedure.

The naked polynucleotides are delivered by any method known in the art, including, but not limited to, direct needle injection at the delivery site, intravenous injection, topical administration, catheter infusion, and so-called “gene guns”. These delivery methods are known in the art.

The constructs may also be delivered with delivery vehicles such as viral sequences, viral particles, liposome formulations, lipofectin, precipitating agents, etc. Such methods of delivery are known in the art.

In certain embodiments, the polynucleotide constructs of the invention are complexed in a liposome preparation. Liposomal preparations for use in the instant invention include cationic (positively charged), anionic (negatively charged) and neutral preparations. However, cationic liposomes are particularly preferred because a tight charge complex can be formed between the cationic liposome and the polyanionic nucleic acid. Cationic liposomes have been shown to mediate intracellular delivery of plasmid DNA (Felgner et al., Proc. Natl. Acad. Sci. USA 84:7413-7416 (1987), which is herein incorporated by reference); mRNA (Malone et al., Proc. Natl. Acad. Sci. USA , 86:6077-6081 (1989), which is herein incorporated by reference); and purified transcription factors (Debs et al., J. Biol. Chem., 265:10189-10192 (1990), which is herein incorporated by reference), in functional form.

Cationic liposomes are readily available. For example, N[1-2,3-dioleyloxy)propyl]-N,N,N-triethylammonium (DOTMA) liposomes are particularly useful and are available under the trademark Lipofectin, from GIBCO BRL, Grand Island, N.Y. (See, also, Felgner et al., Proc. Natl. Acad. Sci. USA , 84:7413-7416 (1987), which is herein incorporated by reference). Other commercially available liposomes include transfectace (DDAB/DOPE) and DOTAP/DOPE (Boehringer).

Other cationic liposomes can be prepared from readily available materials using techniques well known in the art. See, e.g. PCT Publication NO: WO 90/11092 (which is herein incorporated by reference) for a description of the synthesis of DOTAP (1,2-bis(oleoyloxy)-3-(trimethylammonio)propane) liposomes. Preparation of DOTMA liposomes is explained in the literature, see, e.g., Felgner et al., Proc. Natl. Acad. Sci. USA, 84:7413-7417, which is herein incorporated by reference. Similar methods can be used to prepare liposomes from other cationic lipid materials.

Similarly, anionic and neutral liposomes are readily available, such as from Avanti Polar Lipids (Birmingham, Ala.), or can be easily prepared using readily available materials. Such materials include phosphatidyl, choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

For example, commercially dioleoylphosphatidyl choline (DOPC), dioleoylphosphatidyl glycerol (DOPG), and dioleoylphosphatidyl ethanolamine (DOPE) can be used in various combinations to make conventional liposomes, with or without the addition of cholesterol. Thus, for example, DOPG/DOPC vesicles can be prepared by drying 50 mg each of DOPG and DOPC under a stream of nitrogen gas into a sonication vial. The sample is placed under a vacuum pump overnight and is hydrated the following day with deionized water. The sample is then sonicated for 2 hours in a capped vial, using a Heat Systems model 350 sonicator equipped with an inverted cup (bath type) probe at the maximum setting while the bath is circulated at 15EC. Alternatively, negatively charged vesicles can be prepared without sonication to produce multilamellar vesicles or by extrusion through nucleopore membranes to produce unilamellar vesicles of discrete size. Other methods are known and available to those of skill in the art.

The liposomes can comprise multilamellar vesicles (MLVs), small unilamellar vesicles (SUVs), or large unilamellar vesicles (LUVs), with SUVs being preferred. The various liposome-nucleic acid complexes are prepared using methods well known in the art. See, e.g., Straubinger et al., Methods of Immunology, 101:512-527 (1983), which is herein incorporated by reference. For example, MLVs containing nucleic acid can be prepared by depositing a thin film of phospholipid on the walls of a glass tube and subsequently hydrating with a solution of the material to be encapsulated. SUVs are prepared by extended sonication of MLVs to produce a homogeneous population of unilamellar liposomes. The material to be entrapped is added to a suspension of preformed MLVs and then sonicated. When using liposomes containing cationic lipids, the dried lipid film is resuspended in an appropriate solution such as sterile water or an isotonic buffer solution such as 10 mM Tris/NaCl, sonicated, and then the preformed liposomes are mixed directly with the DNA. The liposome and DNA form a very stable complex due to binding of the positively charged liposomes to the cationic DNA. SUVs find use with small nucleic acid fragments. LUVs are prepared by a number of methods, well known in the art. Commonly used methods include Ca2+-EDTA chelation (Papahadjopoulos et al., Biochim. Biophys. Acta, 394:483 (1975); Wilson et al., Cell , 17:77 (1979)); ether injection (Deamer et al., Biochim. Biophys. Acta, 443:629 (1976); Ostro et al., Biochem. Biophys. Res. Commun., 76:836 (1977); Fraley et al., Proc. Natl. Acad. Sci. USA, 76:3348 (1979)); detergent dialysis (Enoch et al., Proc. Natl. Acad. Sci. USA , 76:145 (1979)); and reverse-phase evaporation (REV) (Fraley et al., J. Biol. Chem., 255:10431 (1980); Szoka et al., Proc. Natl. Acad. Sci. USA , 75:145 (1978); Schaefer-Ridder et al., Science, 215:166 (1982)), which are herein incorporated by reference.

Generally, the ratio of DNA to liposomes will be from about 10:1 to about 1:10. Preferably, the ration will be from about 5:1 to about 1:5. More preferably, the ration will be about 3:1 to about 1:3. Still more preferably, the ratio will be about 1:1.

U.S. Pat. No.: 5,676,954 (which is herein incorporated by reference) reports on the injection of genetic material, complexed with cationic liposomes carriers, into mice. U.S. Pat. Nos. 4,897,355, 4,946,787, 5,049,386, 5,459,127, 5,589,466, 5,693,622, 5,580,859, 5,703,055, and international publication NO: WO 94/9469 (which are herein incorporated by reference) provide cationic lipids for use in transfecting DNA into cells and mammals. U.S. Pat. Nos. 5,589,466, 5,693,622, 5,580,859, 5,703,055, and international publication NO: WO 94/9469 (which are herein incorporated by reference) provide methods for delivering DNA-cationic lipid complexes to mammals.

In certain embodiments, cells are engineered, ex vivo or in vivo, using a retroviral particle containing RNA which comprises a sequence encoding polypeptides of the invention. Retroviruses from which the retroviral plasmid vectors may be derived include, but are not limited to, Moloney Murine Leukemia Virus, spleen necrosis virus, Rous sarcoma Virus, Harvey Sarcoma Virus, avian leukosis virus, gibbon ape leukemia virus, human immunodeficiency virus, Myeloproliferative Sarcoma Virus, and mammary tumor virus.

The retroviral plasmid vector is employed to transduce packaging cell lines to form producer cell lines. Examples of packaging cells which may be transfected include, but are not limited to, the PE501, PA317, R-2, R-AM, PA12, T19-14X, VT-19-17-H2, RCRE, RCRIP, GP+E-86, GP+envAm12, and DAN cell lines as described in Miller, Human Gene Therapy , 1:5-14 (1990), which is incorporated herein by reference in its entirety. The vector may transduce the packaging cells through any means known in the art. Such means include, but are not limited to, electroporation, the use of liposomes, and CaPO4 precipitation. In one alternative, the retroviral plasmid vector may be encapsulated into a liposome, or coupled to a lipid, and then administered to a host.

The producer cell line generates infectious retroviral vector particles which include polynucleotide encoding polypeptides of the invention. Such retroviral vector particles then may be employed, to transduce eukaryotic cells, either in vitro or in vivo. The transduced eukaryotic cells will express polypeptides of the invention.

In certain other embodiments, cells are engineered, ex vivo or in vivo, with polynucleotides of the invention contained in an adenovirus vector. Adenovirus can be manipulated such that it encodes and expresses polypeptides of the invention, and at the same time is inactivated in terms of its ability to replicate in a normal lytic viral life cycle. Adenovirus expression is achieved without integration of the viral DNA into the host cell chromosome, thereby alleviating concerns about insertional mutagenesis. Furthermore, adenoviruses have been used as live enteric vaccines for many years with an excellent safety profile (Schwartzet al., Am. Rev. Respir. Dis., 109:233-238 (1974)). Finally, adenovirus mediated gene transfer has been demonstrated in a number of instances including transfer of alpha-1-antitrypsin and CFTR to the lungs of cotton rats (Rosenfeld et al., Science, 252:431-434 (1991); Rosenfeld et al., Cell, 68:143-155 (1992)). Furthermore, extensive studies to attempt to establish adenovirus as a causative agent in human cancer were uniformly negative (Green et al. Proc. Natl. Acad. Sci. USA, 76:6606 (1979)).

Suitable adenoviral vectors useful in the present invention are described, for example, in Kozarsky and Wilson, Curr. Opin. Genet. Devel., 3:499-503 (1993); Rosenfeld et al., Cell, 68:143-155 (1992); Engelhardt et al., Human Genet. Ther., 4:759-769 (1993); Yang et al., Nature Genet., 7:362-369 (1994); Wilson et al., Nature, 365:691-692 (1993); and U.S. Pat. No.: 5,652,224, which are herein incorporated by reference. For example, the adenovirus vector Ad2 is useful and can be grown in human 293 cells. These cells contain the E1 region of adenovirus and constitutively express E1a and E1b, which complement the defective adenoviruses by providing the products of the genes deleted from the vector. In addition to Ad2, other varieties of adenovirus (e.g., Ad3, Ad5, and Ad7) are also useful in the present invention.

Preferably, the adenoviruses used in the present invention are replication deficient. Replication deficient adenoviruses require the aid of a helper virus and/or packaging cell line to form infectious particles. The resulting virus is capable of infecting cells and can express a polynucleotide of interest which is operably linked to a promoter, but cannot replicate in most cells. Replication deficient adenoviruses may be deleted in one or more of all or a portion of the following genes: E1a, E1b, E3, E4, E2a, or L1 through L5.

In certain other embodiments, the cells are engineered, ex vivo or in vivo, using an adeno-associated virus (AAV). AAVs are naturally occurring defective viruses that require helper viruses to produce infectious particles (Muzyczka, Curr. Topics in Microbiol. Immunol., 158:97 (1992)). It is also one of the few viruses that may integrate its DNA into non-dividing cells. Vectors containing as little as 300 base pairs of AAV can be packaged and can integrate, but space for exogenous DNA is limited to about 4.5 kb. Methods for producing and using such AAVs are known in the art. See, for example, U.S. Pat. Nos. 5,139,941, 5,173,414, 5,354,678, 5,436,146, 5,474,935, 5,478,745, and 5,589,377.

For example, an appropriate AAV vector for use in the present invention will include all the sequences necessary for DNA replication, encapsidation, and host-cell integration. The polynucleotide construct containing polynucleotides of the invention is inserted into the AAV vector using standard cloning methods, such as those found in Sambrook et al., Molecular Cloning: A Laboratory Manual, Cold Spring Harbor Press (1989). The recombinant AAV vector is then transfected into packaging cells which are infected with a helper virus, using any standard technique, including lipofection, electroporation, calcium phosphate precipitation, etc. Appropriate helper viruses include adenoviruses, cytomegaloviruses, vaccinia viruses, or herpes viruses. Once the packaging cells are transfected and infected, they will produce infectious AAV viral particles which contain the polynucleotide construct of the invention. These viral particles are then used to transduce eukaryotic cells, either ex vivo or in vivo. The transduced cells will contain the polynucleotide construct integrated into its genome, and will express the desired gene product.

Another method of gene therapy involves operably associating heterologous control regions and endogenous polynucleotide sequences (e.g. encoding the polypeptide sequence of interest) via homologous recombination (see, e.g., U.S. Pat. No.: 5,641,670, issued Jun. 24, 1997; International Publication NO: WO 96/29411, published Sep. 26, 1996; International Publication NO: WO 94/12650, published Aug. 4, 1994; Koller et al., Proc. Natl. Acad. Sci. USA, 86:8932-8935 (1989); and Zijlstra et al., Nature, 342:435-438 (1989). This method involves the activation of a gene which is present in the target cells, but which is not normally expressed in the cells, or is expressed at a lower level than desired.

Polynucleotide constructs are made, using standard techniques known in the art, which contain the promoter with targeting sequences flanking the promoter. Suitable promoters are described herein. The targeting sequence is sufficiently complementary to an endogenous sequence to permit homologous recombination of the promoter-targeting sequence with the endogenous sequence. The targeting sequence will be sufficiently near the 5′ end of the desired endogenous polynucleotide sequence so the promoter will be operably linked to the endogenous sequence upon homologous recombination.

The promoter and the targeting sequences can be amplified using PCR. Preferably, the amplified promoter contains distinct restriction enzyme sites on the 5′ and 3′ ends. Preferably, the 3′ end of the first targeting sequence contains the same restriction enzyme site as the 5′ end of the amplified promoter and the 5′ end of the second targeting sequence contains the same restriction site as the 3′ end of the amplified promoter. The amplified promoter and targeting sequences are digested and ligated together.

The promoter-targeting sequence construct is delivered to the cells, either as naked polynucleotide, or in conjunction with transfection-facilitating agents, such as liposomes, viral sequences, viral particles, whole viruses, lipofection, precipitating agents, etc., described in more detail above. The P promoter-targeting sequence can be delivered by any method, included direct needle injection, intravenous injection, topical administration, catheter infusion, particle accelerators, etc. The methods are described in more detail below.

The promoter-targeting sequence construct is taken up by cells. Homologous recombination between the construct and the endogenous sequence takes place, such that an endogenous sequence is placed under the control of the promoter. The promoter then drives the expression of the endogenous sequence.

The polynucleotides encoding polypeptides of the present invention may be administered along with other polynucleotides encoding angiogenic proteins. Angiogenic proteins include, but are not limited to, acidic and basic fibroblast growth factors, VEGF-1, VEGF-2 (VEGF-C), VEGF-3 (VEGF-B), epidermal growth factor alpha and beta, platelet-derived endothelial cell growth factor, platelet-derived growth factor, tumor necrosis factor alpha, hepatocyte growth factor, insulin like growth factor, colony stimulating factor, macrophage colony stimulating factor, granulocyte/macrophage colony stimulating factor, and nitric oxide synthase.

Preferably, the polynucleotide encoding a polypeptide of the invention contains a secretory signal sequence that facilitates secretion of the protein. Typically, the signal sequence is positioned in the coding region of the polynucleotide to be expressed towards or at the 5′ end of the coding region. The signal sequence may be homologous or heterologous to the polynucleotide of interest and may be homologous or heterologous to the cells to be transfected. Additionally, the signal sequence may be chemically synthesized using methods known in the art.

Any mode of administration of any of the above-described polynucleotides constructs can be used so long as the mode results in the expression of one or more molecules in an amount sufficient to provide a therapeutic effect. This includes direct needle injection, systemic injection, catheter infusion, biolistic injectors, particle accelerators (i.e., “gene guns”), gelfoam sponge depots, other commercially available depot materials, osmotic pumps (e.g., Alza minipumps), oral or suppositorial solid (tablet or pill) pharmaceutical formulations, and decanting or topical applications during surgery. For example, direct injection of naked calcium phosphate-precipitated plasmid into rat liver and rat spleen or a protein-coated plasmid into the portal vein has resulted in gene expression of the foreign gene in the rat livers. (Kaneda et al., Science, 243:375 (1989)).

A preferred method of local administration is by direct injection. Preferably, a recombinant molecule of the present invention complexed with a delivery vehicle is administered by direct injection into or locally within the area of arteries. Administration of a composition locally within the area of arteries refers to injecting the composition centimeters and preferably, millimeters within arteries.

Another method of local administration is to contact a polynucleotide construct of the present invention in or around a surgical wound. For example, a patient can undergo surgery and the polynucleotide construct can be coated on the surface of tissue inside the wound or the construct can be injected into areas of tissue inside the wound.

Therapeutic compositions useful in systemic administration, include recombinant molecules of the present invention complexed to a targeted delivery vehicle of the present invention. Suitable delivery vehicles for use with systemic administration comprise liposomes comprising ligands for targeting the vehicle to a particular site.

Preferred methods of systemic administration, include intravenous injection, aerosol, oral and percutaneous (topical) delivery. Intravenous injections can be performed using methods standard in the art. Aerosol delivery can also be performed using methods standard in the art (see, for example, Stribling et al., Proc. Natl. Acad. Sci. USA, 189:11277-11281 (1992), which is incorporated herein by reference). Oral delivery can be performed by complexing a polynucleotide construct of the present invention to a carrier capable of withstanding degradation by digestive enzymes in the gut of an animal. Examples of such carriers, include plastic capsules or tablets, such as those known in the art. Topical delivery can be performed by mixing a polynucleotide construct of the present invention with a lipophilic reagent (e.g., DMSO) that is capable of passing into the skin.

Determining an effective amount of substance to be delivered can depend upon a number of factors including, for example, the chemical structure and biological activity of the substance, the age and weight of the animal, the precise condition requiring treatment and its severity, and the route of administration. The frequency of treatments depends upon a number of factors, such as the amount of polynucleotide constructs administered per dose, as well as the health and history of the subject. The precise amount, number of doses, and timing of doses will be determined by the attending physician or veterinarian. Therapeutic compositions of the present invention can be administered to any animal, preferably to mammals and birds. Preferred mammals include humans, dogs, cats, mice, rats, rabbits sheep, cattle, horses and pigs, with humans being particularly preferred.

### Biological Activities

The polynucleotides or polypeptides, or agonists or antagonists of the present invention can be used in assays to test for one or more biological activities. If these polynucleotides and polypeptides do exhibit activity in a particular assay, it is likely that these molecules may be involved in the diseases associated with the biological activity. Thus, the polynucleotides or polypeptides, or agonists or antagonists could be used to treat the associated disease.

### Hyperproliferative Disorders

A polynucleotides or polypeptides, or agonists or antagonists of the invention can be used to treat, prevent, and/or diagnose hyperproliferative diseases, disorders, and/or conditions, including neoplasms. A polynucleotides or polypeptides, or agonists or antagonists of the present invention may inhibit the proliferation of the disorder through direct or indirect interactions. Alternatively, a polynucleotides or polypeptides, or agonists or antagonists of the present invention may proliferate other cells which can inhibit the hyperproliferative disorder.

For example, by increasing an immune response, particularly increasing antigenic qualities of the hyperproliferative disorder or by proliferating, differentiating, or mobilizing T-cells, hyperproliferative diseases, disorders, and/or conditions can be treated, prevented, and/or diagnosed. This immune response may be increased by either enhancing an existing immune response, or by initiating a new immune response. Alternatively, decreasing an immune response may also be a method of treating, preventing, and/or diagnosing hyperproliferative diseases, disorders, and/or conditions, such as a chemotherapeutic agent.

Examples of hyperproliferative diseases, disorders, and/or conditions that can be treated, prevented, and/or diagnosed by polynucleotides or polypeptides, or agonists or antagonists of the present invention include, but are not limited to neoplasms located in the: colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital.

Similarly, other hyperproliferative diseases, disorders, and/or conditions can also be treated, prevented, and/or diagnosed by a polynucleotides or polypeptides, or agonists or antagonists of the present invention. Examples of such hyperproliferative diseases, disorders, and/or conditions include, but are not limited to: hypergammaglobulinemia, lymphoproliferative diseases, disorders, and/or conditions, paraproteinemias, purpura, sarcoidosis, Sezary Syndrome, Waldenstron's Macroglobulinemia, Gaucher's Disease, histiocytosis, and any other hyperproliferative disease, besides neoplasia, located in an organ system listed above.

One preferred embodiment utilizes polynucleotides of the present invention to inhibit aberrant cellular division, by gene therapy using the present invention, and/or protein fusions or fragments thereof.

Thus, the present invention provides a method for treating or preventing cell proliferative diseases, disorders, and/or conditions by inserting into an abnormally proliferating cell a polynucleotide of the present invention, wherein said polynucleotide represses said expression.

Another embodiment of the present invention provides a method of treating or preventing cell-proliferative diseases, disorders, and/or conditions in individuals comprising administration of one or more active gene copies of the present invention to an abnormally proliferating cell or cells. In a preferred embodiment, polynucleotides of the present invention is a DNA construct comprising a recombinant expression vector effective in expressing a DNA sequence encoding said polynucleotides. In another preferred embodiment of the present invention, the DNA construct encoding the polynucleotides of the present invention is inserted into cells to be treated utilizing a retrovirus, or more Preferably an adenoviral vector (See G J. Nabel, et. al., PNAS 1999 96: 324-326, which is hereby incorporated by reference). In a most preferred embodiment, the viral vector is defective and will not transform non-proliferating cells, only proliferating cells. Moreover, in a preferred embodiment, the polynucleotides of the present invention inserted into proliferating cells either alone, or in combination with or fused to other polynucleotides, can then be modulated via an external stimulus (i.e. magnetic, specific small molecule, chemical, or drug administration, etc.), which acts upon the promoter upstream of said polynucleotides to induce expression of the encoded protein product. As such the beneficial therapeutic affect of the present invention may be expressly modulated (i.e. to increase, decrease, or inhibit expression of the present invention) based upon said external stimulus.

For local administration to abnormally proliferating cells, polynucleotides of the present invention may be administered by any method known to those of skill in the art including, but not limited to transfection, electroporation, microinjection of cells, or in vehicles such as liposomes, lipofectin, or as naked polynucleotides, or any other method described throughout the specification. The polynucleotide of the present invention may be delivered by known gene delivery systems such as, but not limited to, retroviral vectors (Gilboa, J. Virology 44:845 (1982); Hocke, Nature 320:275 (1986); Wilson, et al., Proc. Natl. Acad. Sci. U.S.A. 85:3014), vaccinia virus system (Chakrabarty et al., Mol. Cell Biol. 5:3403 (1985) or other efficient DNA delivery systems (Yates et al., Nature 313:812 (1985)) known to those skilled in the art. These references are exemplary only and are hereby incorporated by reference. In order to specifically deliver or transfect cells which are abnormally proliferating and spare non-dividing cells, it is preferable to utilize a retrovirus, or adenoviral (as described in the art and elsewhere herein) delivery system known to those of skill in the art. Since host DNA replication is required for retroviral DNA to integrate and the retrovirus will be unable to self replicate due to the lack of the retrovirus genes needed for its life cycle. Utilizing such a retroviral delivery system for polynucleotides of the present invention will target said gene and constructs to abnormally proliferating cells and will spare the non-dividing normal cells.

The polynucleotides of the present invention may be delivered directly to cell proliferative disorder/disease sites in internal organs, body cavities and the like by use of imaging devices used to guide an injecting needle directly to the disease site. The polynucleotides of the present invention may also be administered to disease sites at the time of surgical intervention.

By “cell proliferative disease” is meant any human or animal disease or disorder, affecting any one or any combination of organs, cavities, or body parts, which is characterized by single or multiple local abnormal proliferations of cells, groups of cells, or tissues, whether benign or malignant.

Any amount of the polynucleotides of the present invention may be administered as long as it has a biologically inhibiting effect on the proliferation of the treated cells. Moreover, it is possible to administer more than one of the polynucleotide of the present invention simultaneously to the same site. By “biologically inhibiting” is meant partial or total growth inhibition as well as decreases in the rate of proliferation or growth of the cells. The biologically inhibitory dose may be determined by assessing the effects of the polynucleotides of the present invention on target malignant or abnormally proliferating cell growth in tissue culture, tumor growth in animals and cell cultures, or any other method known to one of ordinary skill in the art.

The present invention is further directed to antibody-based therapies which involve administering of anti-polypeptides and anti-polynucleotide antibodies to a mammalian, preferably human, patient for treating, preventing, and/or diagnosing one or more of the described diseases, disorders, and/or conditions. Methods for producing anti-polypeptides and anti-polynucleotide antibodies polyclonal and monoclonal antibodies are described in detail elsewhere herein. Such antibodies may be provided in pharmaceutically acceptable compositions as known in the art or as described herein.

A summary of the ways in which the antibodies of the present invention may be used therapeutically includes binding polynucleotides or polypeptides of the present invention locally or systemically in the body or by direct cytotoxicity of the antibody, e.g. as mediated by complement (CDC) or by effector cells (ADCC). Some of these approaches are described in more detail below. Armed with the teachings provided herein, one of ordinary skill in the art will know how to use the antibodies of the present invention for diagnostic, monitoring or therapeutic purposes without undue experimentation.

In particular, the antibodies, fragments and derivatives of the present invention are useful for treating, preventing, and/or diagnosing a subject having or developing cell proliferative and/or differentiation diseases, disorders, and/or conditions as described herein. Such treatment comprises administering a single or multiple doses of the antibody, or a fragment, derivative, or a conjugate thereof.

The antibodies of this invention may be advantageously utilized in combination with other monoclonal or chimeric antibodies, or with lymphokines or hematopoietic growth factors, for example, which serve to increase the number or activity of effector cells which interact with the antibodies.

It is preferred to use high affinity and/or potent in vivo inhibiting and/or neutralizing antibodies against polypeptides or polynucleotides of the present invention, fragments or regions thereof, for both immunoassays directed to and therapy of diseases, disorders, and/or conditions related to polynucleotides or polypeptides, including fragments thereof, of the present invention. Such antibodies, fragments, or regions, will preferably have an affinity for polynucleotides or polypeptides, including fragments thereof. Preferred binding affinities include those with a dissociation constant or Kd less than 5×10-6M, 10-6M, 5×10-7M, 10-7M, 5×10-8M, 10-8M, 5×10-9M, 10-9M, 5×10-10M, 10-10M, 5×10-11M, 10-11M, 5×10-12M, 10-12M, 5×10-13M, 10-13M, 5×10-14M, 10-14M, 5×10-15M, and 10-15M.

Moreover, polypeptides of the present invention may be useful in inhibiting the angiogenesis of proliferative cells or tissues, either alone, as a protein fusion, or in combination with other polypeptides directly or indirectly, as described elsewhere herein. In a most preferred embodiment, said anti-angiogenesis effect may be achieved indirectly, for example, through the inhibition of hematopoietic, tumor-specific cells, such as tumor-associated macrophages (See Joseph 1IB, et al. J Natl Cancer Inst, 90(21):1648-53 (1998), which is hereby incorporated by reference). Antibodies directed to polypeptides or polynucleotides of the present invention may also result in inhibition of angiogenesis directly, or indirectly (See Witte L, et al., Cancer Metastasis Rev. 17(2):155-61 (1998), which is hereby incorporated by reference)).

### Cardiovascular Disorders

Polynucleotides or polypeptides, or agonists or antagonists of the invention may be used to treat, prevent, and/or diagnose cardiovascular diseases, disorders, and/or conditions, including peripheral artery disease, such as limb ischemia.

Cardiovascular diseases, disorders, and/or conditions include cardiovascular abnormalities, such as arterio-arterial fistula, arteriovenous fistula, cerebral arteriovenous malformations, congenital heart defects, pulmonary atresia, and Scimitar Syndrome. Congenital heart defects include aortic coarctation, cor triatriatum, coronary vessel anomalies, crisscross heart, dextrocardia, patent ductus arteriosus, Ebstein's anomaly, Eisenmenger complex, hypoplastic left heart syndrome, levocardia, tetralogy of fallot, transposition of great vessels, double outlet right ventricle, tricuspid atresia, persistent truncus arteriosus, and heart septal defects, such as aortopulmonary septal defect, endocardial cushion defects, Lutembacher's Syndrome, trilogy of Fallot, ventricular heart septal defects.

Cardiovascular diseases, disorders, and/or conditions also include heart disease, such as arrhythmias, carcinoid heart disease, high cardiac output, low cardiac output, cardiac tamponade, endocarditis (including bacterial), heart aneurysm, cardiac arrest, congestive heart failure, congestive cardiomyopathy, paroxysmal dyspnea, cardiac edema, heart hypertrophy, congestive cardiomyopathy, left ventricular hypertrophy, right ventricular hypertrophy, post-infarction heart rupture, ventricular septal rupture, heart valve diseases, myocardial diseases, myocardial ischemia, pericardial effusion, pericarditis (including constrictive and tuberculous), pneumopericardium, postpericardiotomy syndrome, pulmonary heart disease, rheumatic heart disease, ventricular dysfunction, hyperemia, cardiovascular pregnancy complications, Scimitar Syndrome, cardiovascular syphilis, and cardiovascular tuberculosis.

Arrhythmias include sinus arrhythmia, atrial fibrillation, atrial flutter, bradycardia, extrasystole, Adams-Stokes Syndrome, bundle-branch block, sinoatrial block, long QT syndrome, parasystole, Lown-Ganong-Levine Syndrome, Mahaim-type pre-excitation syndrome, Wolff-Parkinson-White syndrome, sick sinus syndrome, tachycardias, and ventricular fibrillation. Tachycardias include paroxysmal tachycardia, supraventricular tachycardia, accelerated idioventricular rhythm, atrioventricular nodal reentry tachycardia, ectopic atrial tachycardia, ectopic junctional tachycardia, sinoatrial nodal reentry tachycardia, sinus tachycardia, Torsades de Pointes, and ventricular tachycardia.

Heart valve disease include aortic valve insufficiency, aortic valve stenosis, hear murmurs, aortic valve prolapse, mitral valve prolapse, tricuspid valve prolapse, mitral valve insufficiency, mitral valve stenosis, pulmonary atresia, pulmonary valve insufficiency, pulmonary valve stenosis, tricuspid atresia, tricuspid valve insufficiency, and tricuspid valve stenosis.

Myocardial diseases include alcoholic cardiomyopathy, congestive cardiomyopathy, hypertrophic cardiomyopathy, aortic subvalvular stenosis, pulmonary subvalvular stenosis, restrictive cardiomyopathy, Chagas cardiomyopathy, endocardial fibroelastosis, endomyocardial fibrosis, Kearns Syndrome, myocardial reperfusion injury, and myocarditis.

Myocardial ischemias include coronary disease, such as angina pectoris, coronary aneurysm, coronary arteriosclerosis, coronary thrombosis, coronary vasospasm, myocardial infarction and myocardial stunning.

Cardiovascular diseases also include vascular diseases such as aneurysms, angiodysplasia, angiomatosis, bacillary angiomatosis, Hippel-Lindau Disease, Klippel-Trenaunay-Weber Syndrome, Sturge-Weber Syndrome, angioneurotic edema, aortic diseases, Takayasu's Arteritis, aortitis, Leriche's Syndrome, arterial occlusive diseases, arteritis, enarteritis, polyarteritis nodosa, cerebrovascular diseases, disorders, and/or conditions, diabetic angiopathies, diabetic retinopathy, embolisms, thrombosis, erythromelalgia, hemorrhoids, hepatic veno-occlusive disease, hypertension, hypotension, ischemia, peripheral vascular diseases, phlebitis, pulmonary veno-occlusive disease, Raynaud's disease, CREST syndrome, retinal vein occlusion, Scimitar syndrome, superior vena cava syndrome, telangiectasia, atacia telangiectasia, hereditary hemorrhagic telangiectasia, varicocele, varicose veins, varicose ulcer, vasculitis, and venous insufficiency.

Aneurysms include dissecting aneurysms, false aneurysms, infected aneurysms, ruptured aneurysms, aortic aneurysms, cerebral aneurysms, coronary aneurysms, heart aneurysms, and iliac aneurysms.

Arterial occlusive diseases include arteriosclerosis, intermittent claudication, carotid stenosis, fibromuscular dysplasias, mesenteric vascular occlusion, Moyamoya disease, renal artery obstruction, retinal artery occlusion, and thromboangiitis obliterans.

Cerebrovascular diseases, disorders, and/or conditions include carotid artery diseases, cerebral amyloid angiopathy, cerebral aneurysm, cerebral anoxia, cerebral arteriosclerosis, cerebral arteriovenous malformation, cerebral artery diseases, cerebral embolism and thrombosis, carotid artery thrombosis, sinus thrombosis, Wallenberg's syndrome, cerebral hemorrhage, epidural hematoma, subdural hematoma, subaraxhnoid hemorrhage, cerebral infarction, cerebral ischemia (including transient), subclavian steal syndrome, periventricular leukomalacia, vascular headache, cluster headache, migraine, and vertebrobasilar insufficiency.

Embolisms include air embolisms, amniotic fluid embolisms, cholesterol embolisms, blue toe syndrome, fat embolisms, pulmonary embolisms, and thromoboembolisms. Thrombosis include coronary thrombosis, hepatic vein thrombosis, retinal vein occlusion, carotid artery thrombosis, sinus thrombosis, Wallenberg's syndrome, and thrombophlebitis.

Ischemia includes cerebral ischemia, ischemic colitis, compartment syndromes, anterior compartment syndrome, myocardial ischemia, reperfusion injuries, and peripheral limb ischemia. Vasculitis includes aortitis, arteritis, Behcet's Syndrome, Churg-Strauss Syndrome, mucocutaneous lymph node syndrome, thromboangiitis obliterans, hypersensitivity vasculitis, Schoenlein-Henoch purpura, allergic cutaneous vasculitis, and Wegener's granulomatosis.

Polynucleotides or polypeptides, or agonists or antagonists of the invention, are especially effective for the treatment of critical limb ischemia and coronary disease.

Polypeptides may be administered using any method known in the art, including, but not limited to, direct needle injection at the delivery site, intravenous injection, topical administration, catheter infusion, biolistic injectors, particle accelerators, gelfoam sponge depots, other commercially available depot materials, osmotic pumps, oral or suppositorial solid pharmaceutical formulations, decanting or topical applications during surgery, aerosol delivery. Such methods are known in the art. Polypeptides of the invention may be administered as part of a Therapeutic, described in more detail below. Methods of delivering polynucleotides of the invention are described in more detail herein.

### Anti-Angiogenesis Activity

The naturally occurring balance between endogenous stimulators and inhibitors of angiogenesis is one in which inhibitory influences predominate. Rastinejad et al., Cell 56:345-355 (1989). In those rare instances in which neovascularization occurs under normal physiological conditions, such as wound healing, organ regeneration, embryonic development, and female reproductive processes, angiogenesis is stringently regulated and spatially and temporally delimited. Under conditions of pathological angiogenesis such as that characterizing solid tumor growth, these regulatory controls fail. Unregulated angiogenesis becomes pathologic and sustains progression of many neoplastic and non-neoplastic diseases. A number of serious diseases are dominated by abnormal neovascularization including solid tumor growth and metastases, arthritis, some types of eye diseases, disorders, and/or conditions, and psoriasis. See, e.g., reviews by Moses et al., Biotech. 9:630-634 (1991); Folkman et al., N. Engl. J. Med., 333:1757-1763 (1995); Auerbach et al., J. Microvasc. Res. 29:401-411 (1985); Folkman, Advances in Cancer Research, eds. Klein and Weinhouse, Academic Press, New York, pp. 175-203 (1985); Patz, Am. J. Opthalmol. 94:715-743 (1982); and Folkman et al., Science 221:719-725 (1983). In a number of pathological conditions, the process of angiogenesis contributes to the disease state. For example, significant data have accumulated which suggest that the growth of solid tumors is dependent on angiogenesis. Folkman and Klagsbrun, Science 235:442-447 (1987).

The present invention provides for treatment of diseases, disorders, and/or conditions associated with neovascularization by administration of the polynucleotides and/or polypeptides of the invention, as well as agonists or antagonists of the present invention. Malignant and metastatic conditions which can be treated with the polynucleotides and polypeptides, or agonists or antagonists of the invention include, but are not limited to, malignancies, solid tumors, and cancers described herein and otherwise known in the art (for a review of such disorders, see Fishman et al., Medicine, 2d Ed., J. B. Lippincott Co., Philadelphia (1985)).Thus, the present invention provides a method of treating, preventing, and/or diagnosing an angiogenesis-related disease and/or disorder, comprising administering to an individual in need thereof a therapeutically effective amount of a polynucleotide, polypeptide, antagonist and/or agonist of the invention. For example, polynucleotides, polypeptides, antagonists and/or agonists may be utilized in a variety of additional methods in order to therapeutically treat or prevent a cancer or tumor. Cancers which may be treated, prevented, and/or diagnosed with polynucleotides, polypeptides, antagonists and/or agonists include, but are not limited to solid tumors, including prostate, lung, breast, ovarian, stomach, pancreas, larynx, esophagus, testes, liver, parotid, biliary tract, colon, rectum, cervix, uterus, endometrium, kidney, bladder, thyroid cancer; primary tumors and metastases; melanomas; glioblastoma; Kaposi's sarcoma; leiomyosarcoma; non- small cell lung cancer; colorectal cancer; advanced malignancies; and blood born tumors such as leukemias. For example, polynucleotides, polypeptides, antagonists and/or agonists may be delivered topically, in order to treat or prevent cancers such as skin cancer, head and neck tumors, breast tumors, and Kaposi's sarcoma.

Within yet other aspects, polynucleotides, polypeptides, antagonists and/or agonists may be utilized to treat superficial forms of bladder cancer by, for example, intravesical administration. Polynucleotides, polypeptides, antagonists and/or agonists may be delivered directly into the tumor, or near the tumor site, via injection or a catheter. Of course, as the artisan of ordinary skill will appreciate, the appropriate mode of administration will vary according to the cancer to be treated. Other modes of delivery are discussed herein.

Polynucleotides, polypeptides, antagonists and/or agonists may be useful in treating, preventing, and/or diagnosing other diseases, disorders, and/or conditions, besides cancers, which involve angiogenesis. These diseases, disorders, and/or conditions include, but are not limited to: benign tumors, for example hemangiomas, acoustic neuromas, neurofibromas, trachomas, and pyogenic granulomas; artheroscleric plaques; ocular angiogenic diseases, for example, diabetic retinopathy, retinopathy of prematurity, macular degeneration, corneal graft rejection, neovascular glaucoma, retrolental fibroplasia, rubeosis, retinoblastoma, uvietis and Pterygia (abnormal blood vessel growth) of the eye; rheumatoid arthritis; psoriasis; delayed wound healing; endometriosis; vasculogenesis; granulations; hypertrophic scars (keloids); nonunion fractures; scleroderma; trachoma; vascular adhesions; myocardial angiogenesis; coronary collaterals; cerebral collaterals; arteriovenous malformations; ischemic limb angiogenesis; Osler-Webber Syndrome; plaque neovascularization; telangiectasia; hemophiliac joints; angiofibroma; fibromuscular dysplasia; wound granulation; Crohn's disease; and atherosclerosis.

For example, within one aspect of the present invention methods are provided for treating, preventing, and/or diagnosing hypertrophic scars and keloids, comprising the step of administering a polynucleotide, polypeptide, antagonist and/or agonist of the invention to a hypertrophic scar or keloid.

Within one embodiment of the present invention polynucleotides, polypeptides, antagonists and/or agonists are directly injected into a hypertrophic scar or keloid, in order to prevent the progression of these lesions. This therapy is of particular value in the prophylactic treatment of conditions which are known to result in the development of hypertrophic scars and keloids (e.g., burns), and is preferably initiated after the proliferative phase has had time to progress (approximately 14 days after the initial injury), but before hypertrophic scar or keloid development. As noted above, the present invention also provides methods for treating, preventing, and/or diagnosing neovascular diseases of the eye, including for example, corneal neovascularization, neovascular glaucoma, proliferative diabetic retinopathy, retrolental fibroplasia and macular degeneration.

Moreover, Ocular diseases, disorders, and/or conditions associated with neovascularization which can be treated, prevented, and/or diagnosed with the polynucleotides and polypeptides of the present invention (including agonists and/or antagonists) include, but are not limited to: neovascular glaucoma, diabetic retinopathy, retinoblastoma, retrolental fibroplasia, uveitis, retinopathy of prematurity macular degeneration, corneal graft neovascularization, as well as other eye inflammatory diseases, ocular tumors and diseases associated with choroidal or iris neovascularization. See, e.g., reviews by Waltman et al., Am. J. Ophthal. 85:704-710 (1978) and Gartner et al., Surv. Ophthal. 22:291-312 (1978).

Thus, within one aspect of the present invention methods are provided for treating or preventing neovascular diseases of the eye such as corneal neovascularization (including corneal graft neovascularization), comprising the step of administering to a patient a therapeutically effective amount of a compound (as described above) to the cornea, such that the formation of blood vessels is inhibited. Briefly, the cornea is a tissue which normally lacks blood vessels. In certain pathological conditions however, capillaries may extend into the cornea from the pericorneal vascular plexus of the limbus. When the cornea becomes vascularized, it also becomes clouded, resulting in a decline in the patient's visual acuity. Visual loss may become complete if the cornea completely opacitates. A wide variety of diseases, disorders, and/or conditions can result in corneal neovascularization, including for example, corneal infections (e.g., trachoma, herpes simplex keratitis, leishmaniasis and onchocerciasis), immunological processes (e.g., graft rejection and Stevens-Johnson's syndrome), alkali burns, trauma, inflammation (of any cause), toxic and nutritional deficiency states, and as a complication of wearing contact lenses.

Within particularly preferred embodiments of the invention, may be prepared for topical administration in saline (combined with any of the preservatives and antimicrobial agents commonly used in ocular preparations), and administered in eyedrop form. The solution or suspension may be prepared in its pure form and administered several times daily. Alternatively, anti-angiogenic compositions, prepared as described above, may also be administered directly to the cornea. Within preferred embodiments, the anti-angiogenic composition is prepared with a muco-adhesive polymer which binds to cornea. Within further embodiments, the anti-angiogenic factors or anti-angiogenic compositions may be utilized as an adjunct to conventional steroid therapy. Topical therapy may also be useful prophylactically in corneal lesions which are known to have a high probability of inducing an angiogenic response (such as chemical burns). In these instances the treatment, likely in combination with steroids, may be instituted immediately to help prevent subsequent complications.

Within other embodiments, the compounds described above may be injected directly into the corneal stroma by an ophthalmologist under microscopic guidance. The preferred site of injection may vary with the morphology of the individual lesion, but the goal of the administration would be to place the composition at the advancing front of the vasculature (i.e., interspersed between the blood vessels and the normal cornea). In most cases this would involve perilimbic corneal injection to “protect” the cornea from the advancing blood vessels. This method may also be utilized shortly after a corneal insult in order to prophylactically prevent corneal neovascularization. In this situation the material could be injected in the perilimbic cornea interspersed between the corneal lesion and its undesired potential limbic blood supply. Such methods may also be utilized in a similar fashion to prevent capillary invasion of transplanted corneas. In a sustained-release form injections might only be required 2-3 times per year. A steroid could also be added to the injection solution to reduce inflammation resulting from the injection itself.

Within another aspect of the present invention, methods are provided for treating or preventing neovascular glaucoma, comprising the step of administering to a patient a therapeutically effective amount of a polynucleotide, polypeptide, antagonist and/or agonist to the eye, such that the formation of blood vessels is inhibited. In one embodiment, the compound may be administered topically to the eye in order to treat or prevent early forms of neovascular glaucoma. Within other embodiments, the compound may be implanted by injection into the region of the anterior chamber angle. Within other embodiments, the compound may also be placed in any location such that the compound is continuously released into the aqueous humor. Within another aspect of the present invention, methods are provided for treating or preventing proliferative diabetic retinopathy, comprising the step of administering to a patient a therapeutically effective amount of a polynucleotide, polypeptide, antagonist and/or agonist to the eyes, such that the formation of blood vessels is inhibited.

Within particularly preferred embodiments of the invention, proliferative diabetic retinopathy may be treated by injection into the aqueous humor or the vitreous, in order to increase the local concentration of the polynucleotide, polypeptide, antagonist and/or agonist in the retina. Preferably, this treatment should be initiated prior to the acquisition of severe disease requiring photocoagulation.

Within another aspect of the present invention, methods are provided for treating or preventing retrolental fibroplasia, comprising the step of administering to a patient a therapeutically effective amount of a polynucleotide, polypeptide, antagonist and/or agonist to the eye, such that the formation of blood vessels is inhibited. The compound may be administered topically, via intravitreous injection and/or via intraocular implants.

Additionally, diseases, disorders, and/or conditions which can be treated, prevented, and/or diagnosed with the polynucleotides, polypeptides, agonists and/or agonists include, but are not limited to, hemangioma, arthritis, psoriasis, angiofibroma, atherosclerotic plaques, delayed wound healing, granulations, hemophilic joints, hypertrophic scars, nonunion fractures, Osler-Weber syndrome, pyogenic granuloma, scleroderma, trachoma, and vascular adhesions.

Moreover, diseases, disorders, and/or conditions and/or states, which can be treated, prevented, and/or diagnosed with the polynucleotides, polypeptides, agonists and/or agonists include, but are not limited to, solid tumors, blood born tumors such as leukemias, tumor metastasis, Kaposi's sarcoma, benign tumors, for example hemangiomas, acoustic neuromas, neurofibromas, trachomas, and pyogenic granulomas, rheumatoid arthritis, psoriasis, ocular angiogenic diseases, for example, diabetic retinopathy, retinopathy of prematurity, macular degeneration, corneal graft rejection, neovascular glaucoma, retrolental fibroplasia, rubeosis, retinoblastoma, and uvietis, delayed wound healing, endometriosis, vascluogenesis, granulations, hypertrophic scars (keloids), nonunion fractures, scleroderma, trachoma, vascular adhesions, myocardial angiogenesis, coronary collaterals, cerebral collaterals, arteriovenous malformations, ischemic limb angiogenesis, Osler-Webber Syndrome, plaque neovascularization, telangiectasia, hemophiliac joints, angiofibroma fibromuscular dysplasia, wound granulation, Crohn's disease, atherosclerosis, birth control agent by preventing vascularization required for embryo implantation controlling menstruation, diseases that have angiogenesis as a pathologic consequence such as cat scratch disease (Rochele minalia quintosa), ulcers (Helicobacter pylori), Bartonellosis and bacillary angiomatosis.

In one aspect of the birth control method, an amount of the compound sufficient to block embryo implantation is administered before or after intercourse and fertilization have occurred, thus providing an effective method of birth control, possibly a “morning after” method. Polynucleotides, polypeptides, agonists and/or agonists may also be used in controlling menstruation or administered as either a peritoneal lavage fluid or for peritoneal implantation in the treatment of endometriosis.

Polynucleotides, polypeptides, agonists and/or agonists of the present invention may be incorporated into surgical sutures in order to prevent stitch granulomas.

Polynucleotides, polypeptides, agonists and/or agonists may be utilized in a wide variety of surgical procedures. For example, within one aspect of the present invention a compositions (in the form of, for example, a spray or film) may be utilized to coat or spray an area prior to removal of a tumor, in order to isolate normal surrounding tissues from malignant tissue, and/or to prevent the spread of disease to surrounding tissues. Within other aspects of the present invention, compositions (e.g., in the form of a spray) may be delivered via endoscopic procedures in order to coat tumors, or inhibit angiogenesis in a desired locale. Within yet other aspects of the present invention, surgical meshes which have been coated with anti- angiogenic compositions of the present invention may be utilized in any procedure wherein a surgical mesh might be utilized. For example, within one embodiment of the invention a surgical mesh laden with an anti-angiogenic composition may be utilized during abdominal cancer resection surgery (e.g., subsequent to colon resection) in order to provide support to the structure, and to release an amount of the anti-angiogenic factor.

Within further aspects of the present invention, methods are provided for treating tumor excision sites, comprising administering a polynucleotide, polypeptide, agonist and/or agonist to the resection margins of a tumor subsequent to excision, such that the local recurrence of cancer and the formation of new blood vessels at the site is inhibited. Within one embodiment of the invention, the anti-angiogenic compound is administered directly to the tumor excision site (e.g., applied by swabbing, brushing or otherwise coating the resection margins of the tumor with the anti-angiogenic compound). Alternatively, the anti-angiogenic compounds may be incorporated into known surgical pastes prior to administration. Within particularly preferred embodiments of the invention, the anti-angiogenic compounds are applied after hepatic resections for malignancy, and after neurosurgical operations.

Within one aspect of the present invention, polynucleotides, polypeptides, agonists and/or agonists may be administered to the resection margin of a wide variety of tumors, including for example, breast, colon, brain and hepatic tumors. For example, within one embodiment of the invention, anti-angiogenic compounds may be administered to the site of a neurological tumor subsequent to excision, such that the formation of new blood vessels at the site are inhibited.

The polynucleotides, polypeptides, agonists and/or agonists of the present invention may also be administered along with other anti-angiogenic factors. Representative examples of other anti-angiogenic factors include: Anti-Invasive Factor, retinoic acid and derivatives thereof, paclitaxel, Suramin, Tissue Inhibitor of Metalloproteinase-1, Tissue Inhibitor of Metalloproteinase-2, Plasminogen Activator Inhibitor-1, Plasminogen Activator Inhibitor-2, and various forms of the lighter “d group” transition metals.

Lighter “d group” transition metals include, for example, vanadium, molybdenum, tungsten, titanium, niobium, and tantalum species. Such transition metal species may form transition metal complexes. Suitable complexes of the above-mentioned transition metal species include oxo transition metal complexes.

Representative examples of vanadium complexes include oxo vanadium complexes such as vanadate and vanadyl complexes. Suitable vanadate complexes include metavanadate and orthovanadate complexes such as, for example, ammonium metavanadate, sodium metavanadate, and sodium orthovanadate. Suitable vanadyl complexes include, for example, vanadyl acetylacetonate and vanadyl sulfate including vanadyl sulfate hydrates such as vanadyl sulfate mono- and trihydrates.

Representative examples of tungsten and molybdenum complexes also include oxo complexes. Suitable oxo tungsten complexes include tungstate and tungsten oxide complexes. Suitable tungstate complexes include ammonium tungstate, calcium tungstate, sodium tungstate dihydrate, and tungstic acid. Suitable tungsten oxides include tungsten (IV) oxide and tungsten (VI) oxide. Suitable oxo molybdenum complexes include molybdate, molybdenum oxide, and molybdenyl complexes. Suitable molybdate complexes include ammonium molybdate and its hydrates, sodium molybdate and its hydrates, and potassium molybdate and its hydrates. Suitable molybdenum oxides include molybdenum (VI) oxide, molybdenum (VI) oxide, and molybdic acid. Suitable molybdenyl complexes include, for example, molybdenyl acetylacetonate. Other suitable tungsten and molybdenum complexes include hydroxo derivatives derived from, for example, glycerol, tartaric acid, and sugars.

A wide variety of other anti-angiogenic factors may also be utilized within the context of the present invention. Representative examples include platelet factor 4; protamine sulphate; sulphated chitin derivatives (prepared from queen crab shells), (Murata et al., Cancer Res. 51:22-26, 1991); Sulphated Polysaccharide Peptidoglycan Complex (SP- PG) (the function of this compound may be enhanced by the presence of steroids such as estrogen, and tamoxifen citrate); Staurosporine; modulators of matrix metabolism, including for example, proline analogs, cishydroxyproline, d,L-3,4-dehydroproline, Thiaproline, alpha,alpha-dipyridyl, aminopropionitrile fumarate; ⁴-propyl-5-(4-pyridinyl)-2(3H)-oxazolone; Methotrexate; Mitoxantrone; Heparin; Interferons; 2 Macroglobulin-serum; ChIMP-3 (Pavloff et al., J. Bio. Chem. 267:17321-17326, 1992); Chymostatin (Tomkinson et al., Biochem J. 286:475-480, 1992); Cyclodextrin Tetradecasulfate; Eponemycin; Camptothecin; Fumagillin (Ingber et al., Nature 348:555-557, 1990); Gold Sodium Thiomalate (“GST”; Matsubara and Ziff, J. Clin. Invest. 79:1440-1446, 1987); anticollagenase-serum; alpha2-antiplasmin (Holmes et al., J. Biol. Chem. 262(4):1659-1664, 1987); Bisantrene (National Cancer Institute); Lobenzarit disodium (N-(2)-carboxyphenyl-4-chloroanthronilic acid disodium or “CCA”; Takeuchi et al., Agents Actions 36:312-316, 1992); Thalidomide; Angostatic steroid; AGM-1470; carboxynaminolmidazole; and metalloproteinase inhibitors such as BB94.

### Diseases at the Cellular Level

Diseases associated with increased cell survival or the inhibition of apoptosis that could be treated, prevented, and/or diagnosed by the polynucleotides or polypeptides and/or antagonists or agonists of the invention, include cancers (such as follicular lymphomas, carcinomas with p53 mutations, and hormone-dependent tumors, including, but not limited to colon cancer, cardiac tumors, pancreatic cancer, melanoma, retinoblastoma, glioblastoma, lung cancer, intestinal cancer, testicular cancer, stomach cancer, neuroblastoma, myxoma, myoma, lymphoma, endothelioma, osteoblastoma, osteoclastoma, osteosarcoma, chondrosarcoma, adenoma, breast cancer, prostate cancer, Kaposi's sarcoma and ovarian cancer); autoimmune diseases, disorders, and/or conditions (such as, multiple sclerosis, Sjogren's syndrome, Hashimoto's thyroiditis, biliary cirrhosis, Behcet's disease, Crohn's disease, polymyositis, systemic lupus erythematosus and immune-related glomerulonephritis and rheumatoid arthritis) and viral infections (such as herpes viruses, pox viruses and adenoviruses), inflammation, graft v. host disease, acute graft rejection, and chronic graft rejection. In preferred embodiments, the polynucleotides or polypeptides, and/or agonists or antagonists of the invention are used to inhibit growth, progression, and/or metastasis of cancers, in particular those listed above.

Additional diseases or conditions associated with increased cell survival that could be treated, prevented or diagnosed by the polynucleotides or polypeptides, or agonists or antagonists of the invention, include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

Diseases associated with increased apoptosis that could be treated, prevented, and/or diagnosed by the polynucleotides or polypeptides, and/or agonists or antagonists of the invention, include AIDS; neurodegenerative diseases, disorders, and/or conditions (such as Alzheimer's disease, Parkinson's disease, Amyotrophic lateral sclerosis, Retinitis pigmentosa, Cerebellar degeneration and brain tumor or prior associated disease); autoimmune diseases, disorders, and/or conditions (such as, multiple sclerosis, Sjogren's syndrome, Hashimoto's thyroiditis, biliary cirrhosis, Behcet's disease, Crohn's disease, polymyositis, systemic lupus erythematosus and immune-related glomerulonephritis and rheumatoid arthritis) myelodysplastic syndromes (such as aplastic anemia), graft v. host disease, ischemic injury (such as that caused by myocardial infarction, stroke and reperfusion injury), liver injury (e.g., hepatitis related liver injury, ischemia/reperfusion injury, cholestosis (bile duct injury) and liver cancer); toxin-induced liver disease (such as that caused by alcohol), septic shock, cachexia and anorexia.

### Wound Healing and Epithelial Cell Proliferation

In accordance with yet a further aspect of the present invention, there is provided a process for utilizing the polynucleotides or polypeptides, and/or agonists or antagonists of the invention, for therapeutic purposes, for example, to stimulate epithelial cell proliferation and basal keratinocytes for the purpose of wound healing, and to stimulate hair follicle production and healing of dermal wounds. Polynucleotides or polypeptides, as well as agonists or antagonists of the invention, may be clinically useful in stimulating wound healing including surgical wounds, excisional wounds, deep wounds involving damage of the dermis and epidermis, eye tissue wounds, dental tissue wounds, oral cavity wounds, diabetic ulcers, dermal ulcers, cubitus ulcers, arterial ulcers, venous stasis ulcers, burns resulting from heat exposure or chemicals, and other abnormal wound healing conditions such as uremia, malnutrition, vitamin deficiencies and complications associated with systemic treatment with steroids, radiation therapy and antineoplastic drugs and antimetabolites. Polynucleotides or polypeptides, and/or agonists or antagonists of the invention, could be used to promote dermal reestablishment subsequent to dermal loss

The polynucleotides or polypeptides, and/or agonists or antagonists of the invention, could be used to increase the adherence of skin grafts to a wound bed and to stimulate re-epithelialization from the wound bed. The following are a non-exhaustive list of grafts that polynucleotides or polypeptides, agonists or antagonists of the invention, could be used to increase adherence to a wound bed: autografts, artificial skin, allografts, autodermic graft, autoepidermic grafts, avacular grafts, Blair-Brown grafts, bone graft, brephoplastic grafts, cutis graft, delayed graft, dermic graft, epidermic graft, fascia graft, full thickness graft, heterologous graft, xenograft, homologous graft, hyperplastic graft, lamellar graft, mesh graft, mucosal graft, Ollier-Thiersch graft, omenpal graft, patch graft, pedicle graft, penetrating graft, split skin graft, thick split graft. The polynucleotides or polypeptides, and/or agonists or antagonists of the invention, can be used to promote skin strength and to improve the appearance of aged skin.

It is believed that the polynucleotides or polypeptides, and/or agonists or antagonists of the invention, will also produce changes in hepatocyte proliferation, and epithelial cell proliferation in the lung, breast, pancreas, stomach, small intestine, and large intestine. The polynucleotides or polypeptides, and/or agonists or antagonists of the invention, could promote proliferation of epithelial cells such as sebocytes, hair follicles, hepatocytes, type II pneumocytes, mucin-producing goblet cells, and other epithelial cells and their progenitors contained within the skin, lung, liver, and gastrointestinal tract. The polynucleotides or polypeptides, and/or agonists or antagonists of the invention, may promote proliferation of endothelial cells, keratinocytes, and basal keratinocytes.

The polynucleotides or polypeptides, and/or agonists or antagonists of the invention, could also be used to reduce the side effects of gut toxicity that result from radiation, chemotherapy treatments or viral infections. The polynucleotides or polypeptides, and/or agonists or antagonists of the invention, may have a cytoprotective effect on the small intestine mucosa. The polynucleotides or polypeptides, and/or agonists or antagonists of the invention, may also stimulate healing of mucositis (mouth ulcers) that result from chemotherapy and viral infections.

The polynucleotides or polypeptides, and/or agonists or antagonists of the invention, could further be used in full regeneration of skin in full and partial thickness skin defects, including burns, (i.e., repopulation of hair follicles, sweat glands, and sebaceous glands), treatment of other skin defects such as psoriasis. The polynucleotides or polypeptides, and/or agonists or antagonists of the invention, could be used to treat epidermolysis bullosa, a defect in adherence of the epidermis to the underlying dermis which results in frequent, open and painful blisters by accelerating reepithelialization of these lesions. The polynucleotides or polypeptides, and/or agonists or antagonists of the invention, could also be used to treat gastric and doudenal ulcers and help heal by scar formation of the mucosal lining and regeneration of glandular mucosa and duodenal mucosal lining more rapidly. Inflamamatory bowel diseases, such as Crohn's disease and ulcerative colitis, are diseases which result in destruction of the mucosal surface of the small or large intestine, respectively. Thus, the polynucleotides or polypeptides, and/or agonists or antagonists of the invention, could be used to promote the resurfacing of the mucosal surface to aid more rapid healing and to prevent progression of inflammatory bowel disease. Treatment with the polynucleotides or polypeptides, and/or agonists or antagonists of the invention, is expected to have a significant effect on the production of mucus throughout the gastrointestinal tract and could be used to protect the intestinal mucosa from injurious substances that are ingested or following surgery. The polynucleotides or polypeptides, and/or agonists or antagonists of the invention, could be used to treat diseases associate with the under expression of the polynucleotides of the invention.

Moreover, the polynucleotides or polypeptides, and/or agonists or antagonists of the invention, could be used to prevent and heal damage to the lungs due to various pathological states. A growth factor such as the polynucleotides or polypeptides, and/or agonists or antagonists of the invention, which could stimulate proliferation and differentiation and promote the repair of alveoli and brochiolar epithelium to prevent or treat acute or chronic lung damage. For example, emphysema, which results in the progressive loss of aveoli, and inhalation injuries, i.e., resulting from smoke inhalation and burns, that cause necrosis of the bronchiolar epithelium and alveoli could be effectively treated, prevented, and/or diagnosed using the polynucleotides or polypeptides, and/or agonists or antagonists of the invention. Also, the polynucleotides or polypeptides, and/or agonists or antagonists of the invention, could be used to stimulate the proliferation of and differentiation of type II pneumocytes, which may help treat or prevent disease such as hyaline membrane diseases, such as infant respiratory distress syndrome and bronchopulmonary displasia, in premature infants.

The polynucleotides or polypeptides, and/or agonists or antagonists of the invention, could stimulate the proliferation and differentiation of hepatocytes and, thus, could be used to alleviate or treat liver diseases and pathologies such as fulminant liver failure caused by cirrhosis, liver damage caused by viral hepatitis and toxic substances (i.e., acetaminophen, carbon tetraholoride and other hepatotoxins known in the art).

In addition, the polynucleotides or polypeptides, and/or agonists or antagonists of the invention, could be used treat or prevent the onset of diabetes mellitus. In patients with newly diagnosed Types I and II diabetes, where some islet cell function remains, the polynucleotides or polypeptides, and/or agonists or antagonists of the invention, could be used to maintain the islet function so as to alleviate, delay or prevent permanent manifestation of the disease. Also, the polynucleotides or polypeptides, and/or agonists or antagonists of the invention, could be used as an auxiliary in islet cell transplantation to improve or promote islet cell function.

### Neurological Diseases

Nervous system diseases, disorders, and/or conditions, which can be treated, prevented, and/or diagnosed with the compositions of the invention (e.g., polypeptides, polynucleotides, and/or agonists or antagonists), include, but are not limited to, nervous system injuries, and diseases, disorders, and/or conditions which result in either a disconnection of axons, a diminution or degeneration of neurons, or demyelination. Nervous system lesions which may be treated, prevented, and/or diagnosed in a patient (including human and non-human mammalian patients) according to the invention, include but are not limited to, the following lesions of either the central (including spinal cord, brain) or peripheral nervous systems: (1) ischemic lesions, in which a lack of oxygen in a portion of the nervous system results in neuronal injury or death, including cerebral infarction or ischemia, or spinal cord infarction or ischemia; (2) traumatic lesions, including lesions caused by physical injury or associated with surgery, for example, lesions which sever a portion of the nervous system, or compression injuries; (3) malignant lesions, in which a portion of the nervous system is destroyed or injured by malignant tissue which is either a nervous system associated malignancy or a malignancy derived from non-nervous system tissue; (4) infectious lesions, in which a portion of the nervous system is destroyed or injured as a result of infection, for example, by an abscess or associated with infection by human immunodeficiency virus, herpes zoster, or herpes simplex virus or with Lyme disease, tuberculosis, syphilis; (5) degenerative lesions, in which a portion of the nervous system is destroyed or injured as a result of a degenerative process including but not limited to degeneration associated with Parkinson's disease, Alzheimer's disease, Huntington's chorea, or amyotrophic lateral sclerosis (ALS); (6) lesions associated with nutritional diseases, disorders, and/or conditions, in which a portion of the nervous system is destroyed or injured by a nutritional disorder or disorder of metabolism including but not limited to, vitamin B12 deficiency, folic acid deficiency, Wernicke disease, tobacco-alcohol amblyopia, Marchiafava-Bignami disease (primary degeneration of the corpus callosum), and alcoholic cerebellar degeneration; (7) neurological lesions associated with systemic diseases including, but not limited to, diabetes (diabetic neuropathy, Bell's palsy), systemic lupus erythematosus, carcinoma, or sarcoidosis; (8) lesions caused by toxic substances including alcohol, lead, or particular neurotoxins; and (9) demyelinated lesions in which a portion of the nervous system is destroyed or injured by a demyelinating disease including, but not limited to, multiple sclerosis, human immunodeficiency virus-associated myelopathy, transverse myelopathy or various etiologies, progressive multifocal leukoencephalopathy, and central pontine myelinolysis.

In a preferred embodiment, the polypeptides, polynucleotides, or agonists or antagonists of the invention are used to protect neural cells from the damaging effects of cerebral hypoxia. According to this embodiment, the′ compositions of the invention are used to treat, prevent, and/or diagnose neural cell injury associated with cerebral hypoxia. In one aspect of this embodiment, the polypeptides, polynucleotides, or agonists or antagonists of the invention are used to treat, prevent, and/or diagnose neural cell injury associated with cerebral ischemia. In another aspect of this embodiment, the polypeptides, polynucleotides, or agonists or antagonists of the invention are used to treat, prevent, and/or diagnose neural cell injury associated with cerebral infarction. In another aspect of this embodiment, the polypeptides, polynucleotides, or agonists or antagonists of the invention are used to treat, prevent, and/or diagnose or prevent neural cell injury associated with a stroke. In a further aspect of this embodiment, the polypeptides, polynucleotides, or agonists or antagonists of the invention are used to treat, prevent, and/or diagnose neural cell injury associated with a heart attack.

The compositions of the invention which are useful for treating or preventing a nervous system disorder may be selected by testing for biological activity in promoting the survival or differentiation of neurons. For example, and not by way of limitation, compositions of the invention which elicit any of the following effects may be useful according to the invention: (1) increased survival time of neurons in culture; (2) increased sprouting of neurons in culture or in vivo; (3) increased production of a neuron-associated molecule in culture or in vivo, e.g., choline acetyltransferase or acetylcholinesterase with respect to motor neurons; or (4) decreased symptoms of neuron dysfunction in vivo. Such effects may be measured by any method known in the art. In preferred, non-limiting embodiments, increased survival of neurons may routinely be measured using a method set forth herein or otherwise known in the art, such as, for example, the method set forth in Arakawa et al. (J. Neurosci. 10:3507-3515 (1990)); increased sprouting of neurons may be detected by methods known in the art, such as, for example, the methods set forth in Pestronk et al. (Exp. Neurol. 70:65-82 (1980)) or Brown et al. (Ann. Rev. Neurosci. 4:17-42 (1981)); increased production of neuron-associated molecules may be measured by bioassay, enzymatic assay, antibody binding, Northern blot assay, etc., using techniques known in the art and depending on the molecule to be measured; and motor neuron dysfunction may be measured by assessing the physical manifestation of motor neuron disorder, e.g., weakness, motor neuron conduction velocity, or functional disability.

In specific embodiments, motor neuron diseases, disorders, and/or conditions that may be treated, prevented, and/or diagnosed according to the invention include, but are not limited to, diseases, disorders, and/or conditions such as infarction, infection, exposure to toxin, trauma, surgical damage, degenerative disease or malignancy that may affect motor neurons as well as other components of the nervous system, as well as diseases, disorders, and/or conditions that selectively affect neurons such as amyotrophic lateral sclerosis, and including, but not limited to, progressive spinal muscular atrophy, progressive bulbar palsy, primary lateral sclerosis, infantile and juvenile muscular atrophy, progressive bulbar paralysis of childhood (Fazio-Londe syndrome), poliomyelitis and the post polio syndrome, and Hereditary Motorsensory Neuropathy (Charcot-Marie-Tooth Disease).

### Infectious Disease

A polypeptide or polynucleotide and/or agonist or antagonist of the present invention can be used to treat, prevent, and/or diagnose infectious agents. For example, by increasing the immune response, particularly increasing the proliferation and differentiation of B and/or T cells, infectious diseases may be treated, prevented, and/or diagnosed. The immune response may be increased by either enhancing an existing immune response, or by initiating a new immune response. Alternatively, polypeptide or polynucleotide and/or agonist or antagonist of the present invention may also directly inhibit the infectious agent, without necessarily eliciting an immune response.

Viruses are one example of an infectious agent that can cause disease or symptoms that can be treated, prevented, and/or diagnosed by a polynucleotide or polypeptide and/or agonist or antagonist of the present invention. Examples of viruses, include, but are not limited to Examples of viruses, include, but are not limited to the following DNA and RNA viruses and viral families: Arbovirus, Adenoviridae, Arenaviridae, Arterivirus, Birnaviridae, Bunyaviridae, Caliciviridae, Circoviridae, Coronaviridae, Dengue, EBV, HIV, Flaviviridae, Hepadnaviridae (Hepatitis), Herpesviridae (such as, Cytomegalovirus, Herpes Simplex, Herpes Zoster), Mononegavirus (e.g., Paramyxoviridae, Morbillivirus, Rhabdoviridae), Orthomyxoviridae (e.g., Influenza A, Influenza B, and parainfluenza), Papiloma virus, Papovaviridae, Parvoviridae, Picornaviridae, Poxviridae (such as Smallpox or Vaccinia), Reoviridae (e.g., Rotavirus), Retroviridae (HTLV-I, HTLV-II, Lentivirus), and Togaviridae (e.g., Rubivirus). Viruses falling within these families can cause a variety of diseases or symptoms, including, but not limited to: arthritis, bronchiollitis, respiratory syncytial virus, encephalitis, eye infections (e.g., conjunctivitis, keratitis), chronic fatigue syndrome, hepatitis (A, B, C, E, Chronic Active, Delta), Japanese B encephalitis, Junin, Chikungunya, Rift Valley fever, yellow fever, meningitis, opportunistic infections (e.g., AIDS), pneumonia, Burkitt's Lymphoma, chickenpox, hemorrhagic fever, Measles, Mumps, Parainfluenza, Rabies, the common cold, Polio, l0 leukemia, Rubella, sexually transmitted diseases, skin diseases (e.g., Kaposi's, warts), and viremia. polynucleotides or polypeptides, or agonists or antagonists of the invention, can be used to treat, prevent, and/or diagnose any of these symptoms or diseases. In specific embodiments, polynucleotides, polypeptides, or agonists or antagonists of the invention are used to treat, prevent, and/or diagnose: meningitis, Dengue, EBV, and/or hepatitis (e.g., hepatitis B). In an additional specific embodiment polynucleotides, polypeptides, or agonists or antagonists of the invention are used to treat patients nonresponsive to one or more other commercially available hepatitis vaccines. In a further specific embodiment polynucleotides, polypeptides, or agonists or antagonists of the invention are used to treat, prevent, and/or diagnose AIDS.

Similarly, bacterial or fungal agents that can cause disease or symptoms and that can be treated, prevented, and/or diagnosed by a polynucleotide or polypeptide and/or agonist or antagonist of the present invention include, but not limited to, include, but not limited to, the following Gram-Negative and Gram-positive bacteria and bacterial families and fungi: Actinomycetales (e.g., Corynebacterium, Mycobacterium, Norcardia), Cryptococcus neoformans, Aspergillosis, Bacillaceae (e.g., Anthrax, Clostridium), Bacteroidaceae, Blastomycosis, Bordetella, Borrelia (e.g., Borrelia burgdorferi), Brucellosis, Candidiasis, Campylobacter, Coccidioidomycosis, Cryptococcosis, Dermatocycoses, E. coli (e.g., Enterotoxigenic E. coli and Enterohemorrhagic E. coli), Enterobacteriaceae (Klebsiella, Salmonella (e.g., Salmonella typhi, and Salmonella paratyphi), Serratia, Yersinia), Erysipelothrix, Helicobacter, Legionellosis, Leptospirosis, Listeria, Mycoplasmatales, Mycobacterium leprae, Vibrio cholerae, Neisseriaceae (e.g., Acinetobacter, Gonorrhea, Menigococcal), Meisseria meningitidis, Pasteurellacea Infections (e.g., Actinobacillus, Heamophilus (e.g., Heamophilus influenza type B), Pasteurella), Pseudomonas, Rickettsiaceae, Chlamydiaceae, Syphilis, Shigella spp., Staphylococcal, Meningiococcal, Pneumococcal and Streptococcal (e.g., Streptococcus pneumoniae and Group B Streptococcus). These bacterial or fungal families can cause the following diseases or symptoms, including, but not limited to: bacteremia, endocarditis, eye infections (conjunctivitis, tuberculosis, uveitis), gingivitis, opportunistic infections (e.g., AIDS related infections), paronychia, prosthesis-related infections, Reiter's Disease, respiratory tract infections, such as Whooping Cough or Empyema, sepsis, Lyme Disease, Cat-Scratch Disease, Dysentery, Paratyphoid Fever, food poisoning, Typhoid, pneumonia, Gonorrhea, meningitis (e.g., mengitis types A and B), Chiamydia, Syphilis, Diphtheria, Leprosy, Paratuberculosis, Tuberculosis, Lupus, Botulism, gangrene, tetanus, impetigo, Rheumatic Fever, Scarlet Fever, sexually transmitted diseases, skin diseases (e.g., cellulitis, dermatocycoses), toxemia, urinary tract infections, wound infections. Polynucleotides or polypeptides, agonists or antagonists of the invention, can be used to treat, prevent, and/or diagnose any of these symptoms or diseases. In specific embodiments, polynucleotides, polypeptides, agonists or antagonists of the invention are used to treat, prevent, and/or diagnose: tetanus, Diptheria, botulism, and/or meningitis type B.

Moreover, parasitic agents causing disease or symptoms that can be treated, prevented, and/or diagnosed by a polynucleotide or polypeptide and/or agonist or antagonist of the present invention include, but not limited to, the following families or class: Amebiasis, Babesiosis, Coccidiosis, Cryptosporidiosis, Dientamoebiasis, Dourine, Ectoparasitic, Giardiasis, Helminthiasis, Leishmaniasis, Theileriasis, Toxoplasmosis, Trypanosomiasis, and Trichomonas and Sporozoans (e.g., Plasmodium virax, Plasmodium falciparium, Plasmodium malariae and Plasmodium ovale). These parasites can cause a variety of diseases or symptoms, including, but not limited to: Scabies, Trombiculiasis, eye infections, intestinal disease (e.g., dysentery, giardiasis), liver disease, lung disease, opportunistic infections (e.g., AIDS related), malaria, pregnancy complications, and toxoplasmosis. polynucleotides or polypeptides, or agonists or antagonists of the invention, can be used totreat, prevent, and/or diagnose any of these symptoms or diseases. In specific embodiments, polynucleotides, polypeptides, or agonists or antagonists of the invention are used to treat, prevent, and/or diagnose malaria.

Preferably, treatment or prevention using a polypeptide or polynucleotide and/or agonist or antagonist of the present invention could either be by administering an effective amount of a polypeptide to the patient, or by removing cells from the patient, supplying the cells with a polynucleotide of the present invention, and returning the engineered cells to the patient (ex vivo therapy). Moreover, the polypeptide or polynucleotide of the present invention can be used as an antigen in a vaccine to raise an immune response against infectious disease.

### Regeneration

A polynucleotide or polypeptide and/or agonist or antagonist of the present invention can be used to differentiate, proliferate, and attract cells, leading to the regeneration of tissues. (See, Science 276:59-87 (1997).) The regeneration of tissues could be used to repair, replace, or protect tissue damaged by congenital defects, trauma (wounds, burns, incisions, or ulcers), age, disease (e.g. osteoporosis, osteocarthritis, periodontal disease, liver failure), surgery, including cosmetic plastic surgery, fibrosis, reperfusion injury, or systemic cytokine damage.

Tissues that could be regenerated using the present invention include organs (e.g., pancreas, liver, intestine, kidney, skin, endothelium), muscle (smooth, skeletal or cardiac), vasculature (including vascular and lymphatics), nervous, hematopoietic, and skeletal (bone, cartilage, tendon, and ligament) tissue. Preferably, regeneration occurs without or decreased scarring. Regeneration also may include angiogenesis.

Moreover, a polynucleotide or polypeptide and/or agonist or antagonist of the present invention may increase regeneration of tissues difficult to heal. For example, increased tendon/ligament regeneration would quicken recovery time after damage. A polynucleotide or polypeptide and/or agonist or antagonist of the present invention could also be used prophylactically in an effort to avoid damage. Specific diseases that could be treated, prevented, and/or diagnosed include of tendinitis, carpal tunnel syndrome, and other tendon or ligament defects. A further example of tissue regeneration of non-healing wounds includes pressure ulcers, ulcers associated with vascular insufficiency, surgical, and traumatic wounds.

Similarly, nerve and brain tissue could also be regenerated by using a polynucleotide or polypeptide and/or agonist or antagonist of the present invention to proliferate and differentiate nerve cells. Diseases that could be treated, prevented, and/or diagnosed using this method include central and peripheral nervous system diseases, neuropathies, or mechanical and traumatic diseases, disorders, and/or conditions (e.g., spinal cord disorders, head trauma, cerebrovascular disease, and stoke). Specifically, diseases associated with peripheral nerve injuries, peripheral neuropathy (e.g., resulting from chemotherapy or other medical therapies), localized neuropathies, and central nervous system diseases (e.g., Alzheimer's disease, Parkinson's disease, Huntington's disease, amyotrophic lateral sclerosis, and Shy-Drager syndrome), could all be treated, prevented, and/or diagnosed using the polynucleotide or polypeptide and/or agonist or antagonist of the present invention.

### Chemotaxis

A polynucleotide or polypeptide and/or agonist or antagonist of the present invention may have chemotaxis activity. A chemotaxic molecule attracts or mobilizes cells (e.g., monocytes, fibroblasts, neutrophils, T-cells, mast cells, eosinophils, epithelial and/or endothelial cells) to a particular site in the body, such as inflammation, infection, or site of hyperproliferation. The mobilized cells can then fight off and/or heal the particular trauma or abnormality.

A polynucleotide or polypeptide and/or agonist or antagonist of the present invention may increase chemotaxic activity of particular cells. These chemotactic molecules can then be used to treat, prevent, and/or diagnose inflammation, infection, hyperproliferative diseases, disorders, and/or conditions, or any immune system disorder by increasing the number of cells targeted to a particular location in the body. For example, chemotaxic molecules can be used to treat, prevent, and/or diagnose wounds and other trauma to tissues by attracting immune cells to the injured location. Chemotactic molecules of the present invention can also attract fibroblasts, which can be used to treat, prevent, and/or diagnose wounds.

It is also contemplated that a polynucleotide or polypeptide and/or agonist or antagonist of the present invention may inhibit chemotactic activity. These molecules could also be used to treat, prevent, and/or diagnose diseases, disorders, and/or conditions. Thus, a polynucleotide or polypeptide and/or agonist or antagonist of the present invention could be used as an inhibitor of chemotaxis.

### Binding Activity

A polypeptide of the present invention may be used to screen for molecules that bind to the polypeptide or for molecules to which the polypeptide binds. The binding of the polypeptide and the molecule may activate (agonist), increase, inhibit (antagonist), or decrease activity of the polypeptide or the molecule bound. Examples of such molecules include antibodies, oligonucleotides, proteins (e.g., receptors),or small molecules.

Preferably, the molecule is closely related to the natural ligand of the polypeptide, e.g., a fragment of the ligand, or a natural substrate, a ligand, a structural or functional mimetic. (See, Coligan et al., Current Protocols in Immunology 1(2):Chapter 5 (1991).) Similarly, the molecule can be closely related to the natural receptor to which the polypeptide binds, or at least, a fragment of the receptor capable of being bound by the polypeptide (e.g., active site). In either case, the molecule can be rationally designed using known techniques.

Preferably, the screening for these molecules involves producing appropriate cells which express the polypeptide, either as a secreted protein or on the cell membrane. Preferred cells include cells from mammals, yeast, Drosophila, or E. coli. Cells expressing the polypeptide (or cell membrane containing the expressed polypeptide) are then preferably contacted with a test compound potentially containing the molecule to observe binding, stimulation, or inhibition of activity of either the polypeptide or the molecule.

The assay may simply test binding of a candidate compound to the polypeptide, wherein binding is detected by a label, or in an assay involving competition with a labeled competitor. Further, the assay may test whether the candidate compound results in a signal generated by binding to the polypeptide.

Alternatively, the assay can be carried out using cell-free preparations, polypeptide/molecule affixed to a solid support, chemical libraries, or natural product mixtures. The assay may also simply comprise the steps of mixing a candidate compound with a solution containing a polypeptide, measuring polypeptide/molecule activity or binding, and comparing the polypeptide/molecule activity or binding to a standard.

Preferably, an ELISA assay can measure polypeptide level or activity in a sample (e.g., biological sample) using a monoclonal or polyclonal antibody. The antibody can measure polypeptide level or activity by either binding, directly or indirectly, to the polypeptide or by competing with the polypeptide for a substrate.

Additionally, the receptor to which a polypeptide of the invention binds can be identified by numerous methods known to those of skill in the art, for example, ligand panning and FACS sorting (Coligan, et al., Current Protocols in Immun., 1(2), Chapter 5, (1991)). For example, expression cloning is employed wherein polyadenylated RNA is prepared from a cell responsive to the polypeptides, for example, NIH3T3 cells which are known to contain multiple receptors for the FGF family proteins, and SC-3 cells, and a cDNA library created from this RNA is divided into pools and used to transfect COS cells or other cells that are not responsive to the polypeptides. Transfected cells which are grown on glass slides are exposed to the polypeptide of the present invention, after they have been labeled. The polypeptides can be labeled by a variety of means including iodination or inclusion of a recognition site for a site-specific protein kinase.

Following fixation and incubation, the slides are subjected to auto-radiographic analysis. Positive pools are identified and sub-pools are prepared and re-transfected using an iterative sub-pooling and re-screening process, eventually yielding a single clones that encodes the putative receptor.

As an alternative approach for receptor identification, the labeled polypeptides can be photoaffinity linked with cell membrane or extract preparations that express the receptor molecule. Cross-linked material is resolved by PAGE analysis and exposed to X-ray film. The labeled complex containing the receptors of the polypeptides can be excised, resolved into peptide fragments, and subjected to protein microsequencing. The amino acid sequence obtained from microsequencing would be used to design a set of degenerate oligonucleotide probes to screen a cDNA library to identify the genes encoding the putative receptors.

Moreover, the techniques of gene-shuffling, motif-shuffling, exon-shuffling, and/or codon-shuffling (collectively referred to as “DNA shuffling”) may be employed to modulate the activities of polypeptides of the invention thereby effectively generating agonists and antagonists of polypeptides of the invention. See generally, U.S. Pat. Nos. 5,605,793, 5,811,238, 5,830,721, 5,834,252, and 5,837,458, and Patten, P. A., et al., Curr. Opinion Biotechnol. 8:724-33 (1997); Harayama, S. Trends Biotechnol. 16(2):76-82 (1998); Hansson, L. O., et al., J. Mol. Biol. 287:265-76 (1999); and Lorenzo, M. M. and Blasco, R. Biotechniques 24(2):308-13 (1998) (each of these patents and publications are hereby incorporated by reference). In one embodiment, alteration of polynucleotides and corresponding polypeptides of the invention may be achieved by DNA shuffling. DNA shuffling involves the assembly of two or more DNA segments into a desired polynucleotide sequence of the invention molecule by homologous, or site-specific, recombination. In another embodiment, polynucleotides and corresponding polypeptides of the invention may be altered by being subjected to random mutagenesis by error-prone PCR, random nucleotide insertion or other methods prior to recombination. In another embodiment, one or more components, motifs, sections, parts, domains, fragments, etc., of the polypeptides of the invention may be recombined with one or more components, motifs, sections, parts, domains, fragments, etc. of one or more heterologous molecules. In preferred embodiments, the heterologous molecules are family members. In further preferred embodiments, the heterologous molecule is a growth factor such as, for example, platelet-derived growth factor (PDGF), insulin-like growth factor (IGF-I), transforming growth factor (TGF)-alpha, epidermal growth factor (EGF), fibroblast growth factor (FGF), TGF-beta, bone morphogenetic protein (BMP)-2, BMP-4, BMP-5, BMP-6, BMP-7, activins A and B, decapentaplegic(dpp), 60A, OP-2, dorsalin, growth differentiation factors (GDFs), nodal, MIS, inhibin-alpha, TGF-betal, TGF-beta2, TGF-beta3, TGF-beta5, and glial-derived neurotrophic factor (GDNF).

Other preferred fragments are biologically active fragments of the polypeptides of the invention. Biologically active fragments are those exhibiting activity similar, but not necessarily identical, to an activity of the polypeptide. The biological activity of the fragments may include an improved desired activity, or a decreased undesirable activity.

Additionally, this invention provides a method of screening compounds to identify those which modulate the action of the polypeptide of the present invention. An example of such an assay comprises combining a mammalian fibroblast cell, a the polypeptide of the present invention, the compound to be screened and 3[H] thymidine under cell culture conditions where the fibroblast cell would normally proliferate. A control assay may be performed in the absence of the compound to be screened and compared to the amount of fibroblast proliferation in the presence of the compound to determine if the compound stimulates proliferation by determining the uptake of 3[H] thymidine in each case. The amount of fibroblast cell proliferation is measured by liquid scintillation chromatography which measures the incorporation of 3[H] thymidine. Both agonist and antagonist compounds may be identified by this procedure.

In another method, a mammalian cell or membrane preparation expressing a receptor for a polypeptide of the present invention is incubated with a labeled polypeptide of the present invention in the presence of the compound. The ability of the compound to enhance or block this interaction could then be measured. Alternatively, the response of a known second messenger system following interaction of a compound to be screened and the receptor is measured and the ability of the compound to bind to the receptor and elicit a second messenger response is measured to determine if the compound is a potential agonist or antagonist. Such second messenger systems include but are not limited to, cAMP guanylate cyclase, ion channels or phosphoinositide hydrolysis.

All of these above assays can be used as diagnostic or prognostic markers. The molecules discovered using these assays can be used to treat, prevent, and/or diagnose disease or to bring about a particular result in a patient (e.g., blood vessel growth) by activating or inhibiting the polypeptide/molecule. Moreover, the assays can discover agents which may inhibit or enhance the production of the polypeptides of the invention from suitably manipulated cells or tissues. Therefore, the invention includes a method of identifying compounds which bind to the polypeptides of the invention comprising the steps of: (a) incubating a candidate binding compound with the polypeptide; and (b) determining if binding has occurred. Moreover, the invention includes a method of identifying agonists/antagonists comprising the steps of: (a) incubating a candidate compound with the polypeptide, (b) assaying a biological activity, and (b) determining if a biological activity of the polypeptide has been altered.

Also, one could identify molecules bind a polypeptide of the invention experimentally by using the beta-pleated sheet regions contained in the polypeptide sequence of the protein. Accordingly, specific embodiments of the invention are directed to polynucleotides encoding polypeptides which comprise, or alternatively consist of, the amino acid sequence of each beta pleated sheet regions in a disclosed polypeptide sequence. Additional embodiments of the invention are directed to polynucleotides encoding polypeptides which comprise, or alternatively consist of, any combination or all of contained in the polypeptide sequences of the invention. Additional preferred embodiments of the invention are directed to polypeptides which comprise, or alternatively consist of, the amino acid sequence of each of the beta pleated sheet regions in one of the polypeptide sequences of the invention. Additional embodiments of the invention are directed to polypeptides which comprise, or alternatively consist of, any combination or all of the beta pleated sheet regions in one of the polypeptide sequences of the invention.

### Targeted Delivery

In another embodiment, the invention provides a method of delivering compositions to targeted cells expressing a receptor for a polypeptide of the invention, or cells expressing a cell bound form of a polypeptide of the invention.

As discussed herein, polypeptides or antibodies of the invention may be associated with heterologous polypeptides, heterologous nucleic acids, toxins, or prodrugs via hydrophobic, hydrophilic, ionic and/or covalent interactions. In one embodiment, the invention provides a method for the specific delivery of compositions of the invention to cells by administering polypeptides of the invention (including antibodies) that are associated with heterologous polypeptides or nucleic acids. In one example, the invention provides a method for delivering a therapeutic protein into the targeted cell. In another example, the invention provides a method for delivering a single stranded nucleic acid (e.g., antisense or ribozymes) or double stranded nucleic acid (e.g., DNA that can integrate into the cell's genome or replicate episomally and that can be transcribed) into the targeted cell.

In another embodiment, the invention provides a method for the specific destruction of cells (e.g., the destruction of tumor cells) by administering polypeptides of the invention (e.g., polypeptides of the invention or antibodies of the invention) in association with toxins or cytotoxic prodrugs.

By “toxin” is meant compounds that bind and activate endogenous cytotoxic effector systems, radioisotopes, holotoxins, modified toxins, catalytic subunits of toxins, or any molecules or enzymes not normally present in or on the surface of a cell that under defined conditions cause the cell's death. Toxins that may be used according to the methods of the invention include, but are not limited to, radioisotopes known in the art, compounds such as, for example, antibodies (or complement fixing containing portions thereof) that bind an inherent or induced endogenous cytotoxic effector system, thymidine kinase, endonuclease, RNAse, alpha toxin, ricin, abrin, Pseudomonas exotoxin A, diphtheria toxin, saporin, momordin, gelonin, pokeweed antiviral protein, alpha-sarcin and cholera toxin. By “cytotoxic prodrug” is meant a non-toxic compound that is converted by an enzyme, normally present in the cell, into a cytotoxic compound. Cytotoxic prodrugs that may be used according to the methods of the invention include, but are not limited to, glutamyl derivatives of benzoic acid mustard alkylating agent, phosphate derivatives of etoposide or mitomycin C, cytosine arabinoside, daunorubisin, and phenoxyacetamide derivatives of doxorubicin.

### Drug Screening

Further contemplated is the use of the polypeptides of the present invention, or the polynucleotides encoding these polypeptides, to screen for molecules which modify the activities of the polypeptides of the present invention. Such a method would include contacting the polypeptide of the present invention with a selected compound(s) suspected of having antagonist or agonist activity, and assaying the activity of these polypeptides following binding.

This invention is particularly useful for screening therapeutic compounds by using the polypeptides of the present invention, or binding fragments thereof, in any of a variety of drug screening techniques. The polypeptide or fragment employed in such a test may be affixed to a solid support, expressed on a cell surface, free in solution, or located intracellularly. One method of drug screening utilizes eukaryotic or prokaryotic host cells which are stably transformed with recombinant nucleic acids expressing the polypeptide or fragment. Drugs are screened against such transformed cells in competitive binding assays. One may measure, for example, the formulation of complexes between the agent being tested and a polypeptide of the present invention.

Thus, the present invention provides methods of screening for drugs or any other agents which affect activities mediated by the polypeptides of the present invention. These methods comprise contacting such an agent with a polypeptide of the present invention or a fragment thereof and assaying for the presence of a complex between the agent and the polypeptide or a fragment thereof, by methods well known in the art. In such a competitive binding assay, the agents to screen are typically labeled. Following incubation, free agent is separated from that present in bound form, and the amount of free or uncomplexed label is a measure of the ability of a particular agent to bind to the polypeptides of the present invention.

Another technique for drug screening provides high throughput screening for compounds having suitable binding affinity to the polypeptides of the present invention, and is described in great detail in European Patent Application 84/03564, published on Sep. 13, 1984, which is incorporated herein by reference herein. Briefly stated, large numbers of different small peptide test compounds are synthesized on a solid substrate, such as plastic pins or some other surface. The peptide test compounds are reacted with polypeptides of the present invention and washed. Bound polypeptides are then detected by methods well known in the art. Purified polypeptides are coated directly onto plates for use in the aforementioned drug screening techniques. In addition, non-neutralizing antibodies may be used to capture the peptide and immobilize it on the solid support.

This invention also contemplates the use of competitive drug screening assays in which neutralizing antibodies capable of binding polypeptides of the present invention specifically compete with a test compound for binding to the polypeptides or fragments thereof. In this manner, the antibodies are used to detect the presence of any peptide which shares one or more antigenic epitopes with a polypeptide of the invention.

### Antisense And Ribozyme (Antagonists)

In specific embodiments, antagonists according to the present invention are nucleic acids corresponding to the sequences contained in SEQ ID NO:5, 7, 9, 11, 13, 15, 17, 19, 21, 23, 25, 27, 29, 31, 33, 35, 37, 39, 41, 43, 45, 47, 49, and/or 603, or the complementary strand thereof. In one embodiment, antisense sequence is generated internally by the organism, in another embodiment, the antisense sequence is separately administered (see, for example, O° Connor, Neurochem., 56:560 (1991). Oligodeoxynucleotides as Antisense Inhibitors of Gene Expression, CRC Press, Boca Raton, Fla. (1988). Antisense technology can be used to control gene expression through antisense DNA or RNA, or through triple-helix formation. Antisense techniques are discussed for example, in Okano, Neurochem., 56:560 (1991); Oligodeoxynucleotides as Antisense Inhibitors of Gene Expression, CRC Press, Boca Raton, Fla. (1988). Triple helix formation is discussed in, for instance, Lee et al., Nucleic Acids Research, 6:3073 (1979); Cooney et al., Science, 241:456 (1988); and Dervan et ,al., Science, 251:1300 (1991). The methods are based on binding of a polynucleotide to a complementary DNA or RNA.

For example, the use of c-myc and c-myb antisense RNA constructs to inhibit the growth of the non-lymphocytic leukemia cell line HL-60 and other cell lines was previously described. (Wickstrom et al. (1988); Anfossi et al. (1989)). These experiments were performed in vitro by incubating cells with the oligoribonucleotide. A similar procedure for in vivo use is described in WO 91/15580. Briefly, a pair of oligonucleotides for a given antisense RNA is produced as follows: A sequence complimentary to the first 15 bases of the open reading frame is flanked by an EcoR1 site on the 5 end and a HindIII site on the 3 end. Next, the pair of oligonucleotides is heated at 90° C. for one minute and then annealed in 2× ligation buffer (20 mM TRIS HCl pH 7.5, 10 mM MgCT2, 10MM dithiothreitol (DTT) and 0.2 mM ATP) and then ligated to the EcoR1/Hind III site of the retroviral vector PMV7 (WO 91/15580).

For example, the 5′ coding portion of a polynucleotide that encodes the mature polypeptide of the present invention may be used to design an antisense RNA oligonucleotide of from about 10 to 40 base pairs in length. A DNA oligonucleotide is designed to be complementary to a region of the gene involved in transcription thereby preventing transcription and the production of the receptor. The antisense RNA oligonucleotide hybridizes to the mRNA in vivo and blocks translation of the mRNA molecule into receptor polypeptide.

In one embodiment, the antisense nucleic acid of the invention is produced intracellularly by transcription from an exogenous sequence. For example, a vector or a portion thereof, is transcribed, producing an antisense nucleic acid (RNA) of the invention. Such a vector would contain a sequence encoding the antisense nucleic acid of the invention. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in vertebrate cells. Expression of the sequence encoding a polypeptide of the invention, or fragments thereof, can be by any promoter known in the art to act in vertebrate, preferably human cells. Such promoters can be inducible or constitutive. Such promoters include, but are not limited to, the SV40 early promoter region (Bernoist and Chambon, Nature, 29:304-310 (1981), the promoter contained in the 3′ long terminal repeat of Rous sarcoma virus (Yamamoto et al., Cell, 22:787-797 (1980), the herpes thymidine promoter (Wagner et al., Proc. Natl. Acad. Sci. U.S.A., 78:1441-1445 (1981), the regulatory sequences of the metallothionein gene (Brinster et al., Nature, 296:39-42 (1982)), etc.

The antisense nucleic acids of the invention comprise a sequence complementary to at least a portion of an RNA transcript of a gene of interest. However, absolute complementarity, although preferred, is not required. A sequence “complementary to at least a portion of an RNA,” referred to herein, means a sequence having sufficient complementarity to be able to hybridize with the RNA, forming a stable duplex; in the case of double stranded antisense nucleic acids of the invention, a single strand of the duplex DNA may thus be tested, or triplex formation may be assayed. The ability to hybridize will depend on both the degree of complementarity and the length of the antisense nucleic acid Generally, the larger the hybridizing nucleic acid, the more base mismatches with a RNA sequence of the invention it may contain and still form a stable duplex (or triplex as the case may be). One skilled in the art can ascertain a tolerable degree of mismatch by use of standard procedures to determine the melting point of the hybridized complex.

Oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. See generally, Wagner, R., Nature, 372:333-335 (1994). Thus, oligonucleotides complementary to either the 5′- or 3′- non-translated, non-coding regions of a polynucleotide sequence of the invention could be used in an antisense approach to inhibit translation of endogenous mRNA. Oligonucleotides complementary to the 5′ untranslated region of the mRNA should include the complement of the AUG start codon. Antisense oligonucleotides complementary to mRNA coding regions are less efficient inhibitors of translation but could be used in accordance with the invention. Whether designed to hybridize to the 5′-, 3′- or coding region of mRNA, antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

The polynucleotides of the invention can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger et al., Proc. Natl. Acad. Sci. U.S.A. 86:6553-6556 (1989); Lemaitre et al., Proc. Natl. Acad. Sci., 84:648-652 (1987); PCT Publication NO: W088/09810, published Dec. 15, 1988) or the blood-brain barrier (see, e.g., PCT Publication NO: W089/10134, published Apr. 25, 1988), hybridization-triggered cleavage agents. (See, e.g., Krol et al., BioTechniques, 6:958-976 (1988)) or intercalating agents. (See, e.g., Zon, Pharm. Res., 5:539-549 (1988)). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1 -methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including, but not limited to, arabinose, 2-fluoroarabinose, xylulose, and hexose.

In yet another embodiment, the antisense oligonucleotide comprises at least one modified phosphate backbone selected from the group including, but not limited to, a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an a-anomeric oligonucleotide. An a-anomeric oligonucleotide forms specific double-stranded hybrids with complementary RNA in which, contrary to the usual b-units, the strands run parallel to each other (Gautier et al., Nucl. Acids Res., 15:6625-6641 (1987)). The oligonucleotide is a 2-0-methylribonucleotide (Inoue et al., Nucl. Acids Res., 15:6131-6148 (1987)), or a chimeric RNA-DNA analogue (Inoue et al., FEBS Lett. 215:327-330 (1987)).

Polynucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. (Nucl. Acids Res., 16:3209 (1988)), methylphosphonate oligonucleotides can be prepared by use of controlled pore glass polymer supports (Sarin et al., Proc. Natl. Acad. Sci. U.S.A., 85:7448-7451 (1988)), etc.

While antisense nucleotides complementary to the coding region sequence of the invention could be used, those complementary to the transcribed untranslated region are most preferred.

Potential antagonists according to the invention also include catalytic RNA, or a ribozyme (See, e.g., PCT International Publication WO 90/11364, published Oct. 4, 1990; Sarver et al, Science, 247:1222-1225 (1990). While ribozymes that cleave mRNA at site specific recognition sequences can be used to destroy mRNAs corresponding to the polynucleotides of the invention, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, Nature, 334:585-591 (1988). There are numerous potential hammerhead ribozyme cleavage sites within each nucleotide sequence disclosed in the sequence listing. Preferably, the ribozyme is engineered so that the cleavage recognition site is located near the 5′ end of the mRNA corresponding to the polynucleotides of the invention; i.e., to increase efficiency and minimize the intracellular accumulation of non-functional mRNA transcripts.

As in the antisense approach, the ribozymes of the invention can be composed of modified oligonucleotides (e.g. for improved stability, targeting, etc.) and should be delivered to cells which express the polynucleotides of the invention in vivo. DNA constructs encoding the ribozyme may be introduced into the cell in the same manner as described above for the introduction of antisense encoding DNA. A preferred method of delivery involves using a DNA construct “encoding” the ribozyme under the control of a strong constitutive promoter, such as, for example, pol III or pol II promoter, so that transfected cells will produce sufficient quantities of the ribozyme to destroy endogenous messages and inhibit translation. Since ribozymes unlike antisense molecules, are catalytic, a lower intracellular concentration is required for efficiency.

Antagonist/agonist compounds may be employed to inhibit the cell growth and proliferation effects of the polypeptides of the present invention on neoplastic cells and tissues, i.e. stimulation of angiogenesis of tumors, and, therefore, retard or prevent abnormal cellular growth and proliferation, for example, in tumor formation or growth.

The antagonist/agonist may also be employed to prevent hyper-vascular diseases, and prevent the proliferation of epithelial lens cells after extracapsular cataract surgery. Prevention of the mitogenic activity of the polypeptides of the present invention may also be desirous in cases such as restenosis after balloon angioplasty.

The antagonist/agonist may also be employed to prevent the growth of scar tissue during wound healing.

The antagonist/agonist may also be employed to treat, prevent, and/or diagnose the diseases described herein.

Thus, the invention provides a method of treating or preventing diseases, disorders, and/or conditions, including but not limited to the diseases, disorders, and/or conditions listed throughout this application, associated with overexpression of a polynucleotide of the present invention by administering to a patient (a) an antisense molecule directed to the polynucleotide of the present invention, and/or (b) a ribozyme directed to the polynucleotide of the present invention. invention, and/or (b) a ribozyme directed to the polynucleotide of the present invention.

### Biotic Associations

A polynucleotide or polypeptide and/or agonist or antagonist of the present invention may increase the organisms ability, either directly or indirectly, to initiate and/or maintain biotic associations with other organisms. Such associations may be symbiotic, nonsymbiotic, endosymbiotic, macrosymbiotic, and/or microsymbiotic in nature. In general, a polynucleotide or polypeptide and/or agonist or antagonist of the present invention may increase the organisms ability to form biotic associations with any member of the fungal, bacterial, lichen, mycorrhizal, cyanobacterial, dinoflaggellate, and/or algal, kingdom, phylums, families, classes, genuses, and/or species.

The mechanism by which a polynucleotide or polypeptide and/or agonist or antagonist of the present invention may increase the host organisms ability, either directly or indirectly, to initiate and/or maintain biotic associations is variable, though may include, modulating osmolarity to desirable levels for the symbiont, modulating pH to desirable levels for the symbiont, modulating secretions of organic acids, modulating the secretion of specific proteins, phenolic compounds, nutrients, or the increased expression of a protein required for host-biotic organisms interactions (e.g., a receptor, ligand, etc.). Additional mechanisms are known in the art and are encompassed by the invention (see, for example, “Microbial Signalling and Communication”, eds., R. England, G. Hobbs, N. Bainton, and D. McL. Roberts, Cambridge University Press, Cambridge, (1999); which is hereby incorporated herein by reference).

In an alternative embodiment, a polynucleotide or polypeptide and/or agonist or antagonist of the present invention may decrease the host organisms ability to form biotic associations with another organism, either directly or indirectly. The mechanism by which a polynucleotide or polypeptide and/or agonist or antagonist of the present invention may decrease the host organisms ability, either directly or indirectly, to initiate and/or maintain biotic associations with another organism is variable, though may include, modulating osmolarity to undesirable levels, modulating pH to undesirable levels, modulating secretions of organic acids, modulating the secretion of specific proteins, phenolic compounds, nutrients, or the decreased expression of a protein required for host-biotic organisms interactions (e.g., a receptor, ligand, etc.). Additional mechanisms are known in the art and are encompassed by the invention (see, for example, “Microbial Signalling and Communication”, eds., R. England, G. Hobbs, N. Bainton, and D. McL. Roberts, Cambridge University Press, Cambridge, (1999); which is hereby incorporated herein by reference).

The hosts ability to maintain biotic associations with a particular pathogen has significant implications for the overall health and fitness of the host. For example, human hosts have symbiosis with enteric bacteria in their gastrointestinal tracts, particularly in the small and large intestine. In fact, bacteria counts in feces of the distal colon often approach 10¹² per milliliter of feces. Examples of bowel flora in the gastrointestinal tract are members of the Enterobacteriaceae, Bacteriodes, in addition to a-hemolytic streptococci, E. coli, Bifobacteria, Anaerobic cocci, Eubacteria, Costridia, lactobacilli, and yeasts. Such bacteria, among other things, assist the host in the assimilation of nutrients by breaking down food stuffs not typically broken down by the hosts digestive system, particularly in the hosts bowel. Therefore, increasing the hosts ability to maintain such a biotic association would help assure proper nutrition for the host.

Aberrations in the enteric bacterial population of mammals, particularly humans, has been associated with the following disorders: diarrhea, ileus, chronic inflammatory disease, bowel obstruction, duodenal diverticula, biliary calculous disease, and malnutrition. A polynucleotide or polypeptide and/or agonist or antagonist of the present invention are useful for treating, detecting, diagnosing, prognosing, and/or ameliorating, either directly or indirectly, and of the above mentioned diseases and/or disorders associated with aberrant enteric flora population.

The composition of the intestinal flora, for example, is based upon a variety of factors, which include, but are not limited to, the age, race, diet, malnutrition, gastric acidity, bile salt excretion, gut motility, and immune mechanisms. As a result, the polynucleotides and polypeptides, including agonists, antagonists, and fragments thereof, may modulate the ability of a host to form biotic associations by affecting, directly or indirectly, at least one or more of these factors.

Although the predominate intestinal flora comprises anaerobic organisms, an underlying percentage represents aerobes (e.g., E. coli). This is significant as such aerobes rapidly become the predominate organisms in intraabdominal infections—effectively becoming opportunistic early in infection pathogenesis. As a result, there is an intrinsic need to control aerobe populations, particularly for immune compromised individuals.

In a preferred embodiment, a polynucleotides and polypeptides, including agonists, antagonists, and fragments thereof, are useful for inhibiting biotic associations with specific enteric symbiont organisms in an effort to control the population of such organisms.

Biotic associations occur not only in the gastrointestinal tract, but also on an in the integument. As opposed to the gastrointestinal flora, the cutaneous flora is comprised almost equally with aerobic and anaerobic organisms. Examples of cutaneous flora are members of the gram-positive cocci (e.g., S. aureus, coagulase-negative staphylococci, micrococcus, M.sedentarius), gram-positive bacilli (e.g., Corynebacterium species, C. minutissimum, Brevibacterium species, Propoionibacterium species, P.acnes), gram-negative bacilli (e.g., Acinebacter species), and fungi (Pityrosporum orbiculare). The relatively low number of flora associated with the integument is based upon the inability of many organisms to adhere to the skin. The organisms referenced above have acquired this unique ability. Therefore, the polynucleotides and polypeptides of the present invention may have uses which include modulating the population of the cutaneous flora, either directly or indirectly.

Aberrations in the cutaneous flora are associated with a number of significant diseases and/or disorders, which include, but are not limited to the following: impetigo, ecthyma, blistering distal dactulitis, pustules, folliculitis, cutaneous abscesses, pitted keratolysis, trichomycosis axcillaris, dermatophytosis complex, axillary odor, erthyrasma, cheesy foot odor, acne, tinea versicolor, seborrheic dermititis, and Pityrosporum folliculitis, to name a few. A polynucleotide or polypeptide and/or agonist or antagonist of the present invention are useful for treating, detecting, diagnosing, prognosing, and/or ameliorating, either directly or indirectly, and of the above mentioned diseases and/or disorders associated with aberrant cutaneous flora population.

Additional biotic associations, including diseases and disorders associated with the aberrant growth of such associations, are known in the art and are encompassed by the invention. See, for example, “Infectious Disease”, Second Edition, Eds., S. L., Gorbach, J. G., Bartlett, and N. R., Blacklow, W.B. Saunders Company, Philadelphia, (1998); which is hereby incorporated herein by reference).

### Pheromones

In another embodiment, a polynucleotide or polypeptide and/or agonist or antagonist of the present invention may increase the organisms ability to synthesize and/or release a pheromone. Such a pheromone may, for example, alter the organisms behavior and/or metabolism.

A polynucleotide or polypeptide and/or agonist or antagonist of the present invention may modulate the biosynthesis and/or release of pheromones, the organisms ability to respond to pheromones (e.g., behaviorally, and/or metabolically), and/or the organisms ability to detect pheromones. Preferably, any of the pheromones, and/or volatiles released from the organism, or induced, by a polynucleotide or polypeptide and/or agonist or antagonist of the invention have behavioral effects the organism.

### Other Activities

The polypeptide of the present invention, as a result of the ability to stimulate vascular endothelial cell growth, may be employed in treatment for stimulating re-vascularization of ischemic tissues due to various disease conditions such as thrombosis, arteriosclerosis, and other cardiovascular conditions. These polypeptide may also be employed to stimulate angiogenesis and limb regeneration, as discussed above.

The polypeptide may also be employed for treating wounds due to injuries, burns, post-operative tissue repair, and ulcers since they are mitogenic to various cells of different origins, such as fibroblast cells and skeletal muscle cells, and therefore, facilitate the repair or replacement of damaged or diseased tissue.

The polypeptide of the present invention may also be employed stimulate neuronal growth and to treat, prevent, and/or diagnose neuronal damage which occurs in certain neuronal disorders or neuro-degenerative conditions such as Alzheimer's disease, Parkinson's disease, and AIDS-related complex. The polypeptide of the invention may have the ability to stimulate chondrocyte growth, therefore, they may be employed to enhance bone and periodontal regeneration and aid in tissue transplants or bone grafts.

The polypeptide of the present invention may be also be employed to prevent skin aging due to sunburn by stimulating keratinocyte growth.

The polypeptide of the invention may also be employed for preventing hair loss, since FGF family members activate hair-forming cells and promotes melanocyte growth. Along the same lines, the polypeptides of the present invention may be employed to stimulate growth and differentiation of hematopoietic cells and bone marrow cells when used in combination with other cytokines.

The polypeptide of the invention may also be employed to maintain organs before transplantation or for supporting cell culture of primary tissues.

The polypeptide of the present invention may also be employed for inducing tissue of mesodermal origin to differentiate in early embryos.

The polypeptide or polynucleotides and/or agonist or antagonists of the present invention may also increase or decrease the differentiation or proliferation of embryonic stem cells, besides, as discussed above, hematopoietic lineage.

The polypeptide or polynucleotides and/or agonist or antagonists of the present invention may also be used to modulate mammalian characteristics, such as body height, weight, hair color, eye color, skin, percentage of adipose tissue, pigmentation, size, and shape (e.g., cosmetic surgery). Similarly, polypeptides or polynucleotides and/or agonist or antagonists of the present invention may be used to modulate mammalian metabolism affecting catabolism, anabolism, processing, utilization, and storage of energy.

Polypeptide or polynucleotides and/or agonist or antagonists of the present invention may be used to change a mammal's mental state or physical state by influencing biorhythms, caricadic rhythms, depression (including depressive diseases, disorders, and/or conditions), tendency for violence, tolerance for pain, reproductive capabilities (preferably by Activin or Inhibin-like activity), hormonal or endocrine levels, appetite, libido, memory, stress, or other cognitive qualities.

Polypeptide or polynucleotides and/or agonist or antagonists of the present invention may also be used to prepare individuals for extraterrestrial travel, low gravity environments, prolonged exposure to extraterrestrial radiation levels, low oxygen levels, reduction of metabolic activity, exposure to extraterrestrial pathogens, etc. Such a use may be administered either prior to an extraterrestrial event, during an extraterrestrial event, or both. Moreover, such a use may result in a number of beneficial changes in the recipient, such as, for example, any one of the following, non-limiting, effects: an increased level of hematopojetic cells, particularly red blood cells which would aid the recipient in coping with low oxygen levels; an increased level of B-cells, T-cells, antigen presenting cells, and/or macrophages, which would aid the recipient in coping with exposure to extraterrestrial pathogens, for example; a temporary (i.e., reversible) inhibition of hematopoictic cell production which would aid the recipient in coping with exposure to extraterrestrial radiation levels; increase and/or stability of bone mass which would aid the recipient in coping with low gravity environments; and/or decreased metabolism which would effectively facilitate the recipients ability to prolong their extraterrestrial travel by any one of the following, non-limiting means: (i) aid, the recipient by decreasing their basal daily energy requirements; (ii) effectively lower the level of oxidative and/or metabolic stress in recipient (i.e., to enable recipient to cope with increased extraterrestial radiation levels by decreasing the level of internal oxidative/metabolic damage acquired during normal basal energy requirements; and/or (iii) enabling recipient to subsist at a lower metabolic temperature (i.e., cryogenic, and/or sub-cryogenic environment).

Polypeptide or polynucleotides and/or agonist or antagonists of the present invention may also be used to increase the efficacy of a pharmaceutical composition, either directly or indirectly. Such a use may be administered in simultaneous conjunction with said pharmaceutical, or separately through either the same or different route of administration (e.g., intravenous for the polynucleotide or polypeptide of the present invention, and orally for the pharmaceutical, among others described herein.).

Polypeptide or polynucleotides and/or agonist or antagonists of the present invention may also be used as a food additive or preservative, such as to increase or decrease storage capabilities, fat content, lipid, protein, carbohydrate, vitamins, minerals, cofactors or other nutritional components.

### EXAMPLES

### Example 1

### Method Of Discovering The Single Nucleotide Polymorphisms (SNPs) Of The Present Invention

Candidate genes for SNP discovery were chosen based upon the knowledge that they transport, either directly, or indirectly, statins. Specifically, the following genes were analyzed for the presence of potential SNPs: OATP2, solute carrier family 21 member 6 protein (HGNC ID: SLC21A6), and cMOAT, ATP-binding cassette sub-family C member 2 protein (HGNC_ID: ABCC2).

SNP discovery was based on comparative DNA sequencing of PCR products derived from genomic DNA from multiple individuals. All the genomic DNA samples were purchased from Coriell Institute (Collingswood, N.J.) unless stated otherwise (see Table VII). 24 samples were obtained from Coriell with the samples representing eight members of each ethnic group: Caucasians, African, American, and Asian (mainly Chinese and Japanese). PCR amplicons were designed to cover the entire coding region of the exons using the Primer3 program (Rozen S 2000). Exon-intron structure of candidate genes and intron sequences were obtained by blastn search of Genbank cDNA sequences against the human genome draft sequences. The sizes of these PCR amplicons varied according to the exon-intron structure. All the samples amplified from genomic DNA (20 ng) in reactions (50 ul) containing 10 mM Tris-Cl pH 8.3, 50 mM KCl, 2.5 mM MgCl₂, 150 uM dNTPs, 3 uM PCR primers, and 3.75 U TaqGold DNA polymerase (PE Biosystems).

PCR was performed in MJ Research Tetrad machines under a cycling condition of 94 degrees 10 min, 30 cycles of 94 degrees 30 sec, 60 degrees 30 sec, and 72 degrees 30 sec, followed by 72 degrees 7 min. PCR products were purified using QIAquick PCR purification kit (Qiagen), and were sequenced by the dye-terminator method using PRISM 3700 automated DNA sequencer (Applied Biosystems, Foster City, Calif.) following the manufacturer's instruction outlined in the Owner's Manual (which is hereby incorporated herein by reference in its entirety). Sequencing results were analyzed for the presence of polymorphisms using PolyPhred software(Nickerson DA 1997; Rieder MJ 1999). All the sequence traces of potential polymorphisms were visually inspected to confirm the presence of SNPs.

DNA sequences of PCR primers and sequencing primers used for SNP discovery are provided in Tables VIII and IX, respectfully.

Alternative methods for identifying SNPs of the present invention are known in the art. One such method involves resequencing of target sequences from individuals of diverse ethnic and geographic backgrounds by hybridization to probes immobilized to microfabricated arrays. The strategy and principles for the design and use of such arrays are generally described in WO 95/11995.

A typical probe array used in such as analysis would have two groups of four sets of probes that respectively tile both strands of a reference sequence. A first probe set comprises a plurality of probes exhibiting perfect complementarily with one of the reference sequences. Each probe in the first probe set has an interrogation position that corresponds to a nucleotide in the reference sequence. That is, the interrogation position is aligned with the corresponding nucleotide in the reference sequence, when the probe and reference sequence are aligned to maximize complementarily between the two. For each probe in the first set, there are three corresponding probes from three additional probe sets. Thus, there are four probes corresponding to each nucleotide in the reference sequence. The probes from the three additional probe sets would be identical to the corresponding probe from the first probe set except at the interrogation position, which occurs in the same position in each of the four corresponding probes from the four probe sets, and is occupied by a different nucleotide in the four probe sets. In the present analysis, probes were nucleotides long. Arrays tiled for multiple different references sequences were included on the same substrate.

Publicly available sequences for a given gene can be assembled into Gap4 (http://www.biozentrum.unibas.ch/-biocomp/staden/Overview.html). PCR primers covering each exon, could be designed, for example, using Primer 3 (httP://www-genome.wi.mit.edu/cgi-bin/primer/primer3.cgi). Primers would not be designed in regions where there are sequence discrepancies between reads. Genomic DNA could be amplified from at least two individuals using 2.5 pmol each primer, 1.5 mM MgCl2, 100 ˜M dNTPs, 0.75 ˜M AmpliTaq GOLD polymerase, and about 19 ng DNA in a 15 ul reaction. Reactions could be assembled using a PACKARD MultiPROBE robotic pipetting station and then put in MJ 96-well tetrad thermocyclers (96° C. for minutes, followed by cycles of 96° C. for seconds, 59° C. for 2 minutes, and 72° C. for 2 minutes). A subset of the PCR assays for each individual could then be run on 3% NuSieve gels in 0.5×TBE to confirm that the reaction worked.

For a given DNA, 5 ul (about 50 ng) of each PCR or RT-PCR product could be pooled (Final volume=150-200 ul). The products can be purified using QiaQuick PCR purification from Qiagen. The samples would then be eluted once in 35 ul sterile water and 4 ul lOX One-Phor-All buffer (Pharmacia). The pooled samples are then digested with 0.2 u DNaseI (Promega) for 10 minutes at 37° C. and then labeled with 0.5 nmols biotin-N6-ddATP and 15 u Terminal Transferase (GibcoBRL Life Technology) for 60 minutes at 37° C. Both fragmentation and labeling reactions could be terminated by incubating the pooled sample for 15 minutes at 100° C.

Low-density DNA chips {Affymetrix, Calif.) may be hybridized following the manufacturer's instructions. Briefly, the hybridization cocktail consisted of 3M TMACI, mM Tris pH 7.8, 0.01% Triton X-100, 100 mg/ml herring sperm DNA {Gibco BRL), 200 pM control biotin-labeled oligo. The processed PCR products are then denatured for 7 minutes at 100° C. and then added to prewarmed { 37° C.) hybridization solution. The chips are hybridized overnight at 44° C. Chips are washed in 1×SSPET and 6×SSPET followed by staining with 2 ug/ml SARPE and 0.5 mg/ml acetylated BSA in 200 ul of 6×SSPET for 8 minutes at room temperature. Chips are scanned using a Molecular Dynamics scanner.

Chip image files may be analyzed using Ulysses {Affymetrix, Calif.) which uses four algorithms to identify potential polymorphisms. Candidate polymorphisms may be visually inspected and assigned a confidence value: where high confidence candidates display all three genotypes, while likely candidates show only two genotypes {homozygous for reference sequence and heterozygous for reference and variant). Some of the candidate polymorphisms may be confirmed by ABI sequencing. Identified polymorphisms could then be compared to several databases to determine if they are novel.

### Example 2

### Method of Determining the Allele Frequency for Each SNP of the Present Invention

Allele frequencies of these polymorphisms may be determined by genotyping the Coriell DNA samples (Table VII; Coriell Institute, Collingswood, N.J.) using FP-TDI assay (Chen X 1999). The ethnicity and Coriell Sample IDs for each of the DNA samples utilized for the present invention are provided in Table VII. Automated genotyping calls may be made with an allele calling software developed by Joel Hirschorn (Whitehead Institute/MIT Center for Genome Research, personal communication).

Briefly, the no template controls (NTCs) are labeled accordingly in column C. The appropriate cells may be completed in column L indicating whether REF (homozygous ROX) or VAR (homozygous TAMRA) are expected to be rare genotypes (<10% of all samples)—the latter is important in helping the program to identify rare homozygotes. The number of 96 well plates genotyped in cell P2 are noted (generally between 0.5 and 4)—the program works best if this is accurate. No more than 384 samples can be analyzed at a time. The pairs of mP values from the LJL may be pasted into columns E and F; making sure there may be no residual data was left at the bottom fewer than 384 data points are provided. The DNA names may be provided in columns A, B or C; column I will be a concatenation of columns A, B and C. In addition, the well numbers for each sample may be also provided in column D.

With the above information provided, the program should automatically cluster the points and identify genotypes. The program works by converting the mP values into polar coordinates (distance from origin and angle from origin) with the angle being on a scale from 0 to 2; heterozygotes are placed as close to 1 as possible.

The cutoff values in columns L and M may be adjusted as desired.

Expert parameters: The most important parameters are the maximum angle for REF and minimum angle for VAR. These parameters may need to be changed in a particularly skewed assay which may be observed when an REF or VAR cluster is close to an angle of 1 and has called as a failed or HETs.

Other parameters are low and high cutoffs that are used to determine which points are considered for the determination of edges of the clusters. With small numbers of data points, the high cutoff may need to be increased (to 500 or so). This may be the right thing to do for every assay, but certainly when the program fails to identify a small cluster with high signal.

NTC TAMRA and ROX indicate the position of the no template control or failed samples as estimated by the computer algorithm.

No signal=mP< is the threshold below which points are automatically considered failures. “Throw out points with signal above” is the TAMRA or ROX mP value above which points are considered failures. The latter may occasionally need to be adjusted from 250 to 300, but caveat emptor for assays with signals >250. ‘Lump’ or ‘split’ describes a subtle difference in the way points are grouped into clusters. Lump generally is better. ‘HETs expected’ in the rare case where only homozygotes of either class are expected (e.g. a study of X chromosome SNPs in males), change this to “N”.

Notes on method of clustering: The origin is defined by the NTCs or other low signal points (the position of the origin is shown as “NTC TAMRA” and “NTC ROX”); the points with very low or high signal are not considered initially. The program finds the point farthest from the origin and calls that a HET; the ROX/TAMRA ratio is calculated from this point, placing the heterozygotes at 45 degrees from the origin (an angle of “1”). The angles from the origin are calculated (the scale ranges from 0 to 2) and used to define clusters. A histogram of angles is generated. The cluster boundaries are defined by an algorithm that takes into account the shape of the histogram. The homozygote clusters are defined as the leftmost and rightmost big clusters (unless the allele is specified as being rare, in which case the cluster need not be big). The heterozygote is the biggest cluster in between the REF and VAR. If there are two equal clusters, the one best-separated from REF and VAR is called HET. All other clusters are failed. Some fine tuning is applied to lump in scattered points on the edges of the clusters (if “Lump” is selected). The boundaries of the clusters are “Angles” in column L.

Once the clusters are defined, the interquartile distance of signal intensity is defined for each cluster. Points falling more than 3 or 4 interquartiles from the mean are excluded. (These are the “Signal cutoffs” in column M)

The invention encompasses additional methods of determining the allelic frequency of the SNPs of the present invention. Such methods may be known in the art, some of which are described elsewhere herein.

### Example 3

### Method of Genotyping Each SNP of the Present Invention

### a.) Genomic DNA Preparation

Genomic DNA samples for genotyping may be prepared using the Purigene™ DNA extraction kit from Gentra Systems (http://www.gentra.com). After preparation, DNA samples may be diluted to a 2 ng/ul working concentration with TE buffer (10 mM Tris-Cl, pH 8.0, 0.1 mM EDTA, pH 8.0) and stored in 1 ml 96 deep well plates (VWR) at −20 degrees until use.

Samples for genomic DNA preparation may be obtained from the Coriell tissues sources described herein, or from other sources known in the art or otherwise described herein.

### b) Genotyping

The SNP genotyping reaction may be performed using the SNPStream™ system (Orchid Biosience, Princeton, N.J.) based on genetic bit analysis (Nikiforov, T. et al, Nucleic Acids Res 22, 4167-4175 (1994)).

The regions including polymorphic sites are amplified by the polymerase chain reaction (PCR) using a pair of primers (OPERON Technologies), one of which was phosphorothioated. 6 ul PCR cocktail containing 1.0 ng/ul genomic DNA, 200 uM dNTPs, 0.5 uM forward PCR primer, 0.5 uM reverse PCR primer (phosphorothioated), 0.05 u/ul Platinum Taq DNA polymerase (LifeTechnologies), and 1.5 mM MgCl₂ The PCR reaction may be set up in 384-well plates (MJ Research) using a MiniTrak liquid handling station (Packard Bioscience). The PCR primer sequences may be selected from those provided in Table IX herein, or any other primer as may otherwise be required. PCR thermocycling may be performed under the following conditions in a MJ Research Tetrad machine: step 1, 95 degrees for 2 min; step 2, 94 degrees for 30 min; step 3, 55 degrees for 2 min; step 4, 72 degrees for 30 sec; step 5, go back to step 2 for an additional 39 cycles; step 6, 72 degrees for 1 min; and step 7, 12 degrees indefinitely)

After thermocycling, the amplified samples are placed in the SNPStream™ (Orchid Bioscience) machine, and automated genetic bit analysis (GBA) (Nikiforov, T. et al,supra) reaction is performed. The first step of this reaction is degradation of one of the strands of the PCR products by T7 gene 6 exonuclease to make them single-stranded. The strand containing phosphorothioated primer is resistant to T7 gene 6 nuclease, and is not degraded by this enzyme. After digestion, the single-stranded PCR products are subjected to an annealing step to the GBA primer on a solid phase, and then subjected to the GBA reaction (single base extension) using dideoxy-NTPs labeled with biotin or fluorescein. Incorporation of these dideoxynucleotides into a GBA primer is detected by two color ELISA assay using anti-fluorescein alkaline phosphatase conjugate and anti-biotin horseradish peroxidase. Automated genotype calls are made by GenoPak software (Orchid Bioscience), before manual correction of automated calls are done upon inspection of the resulting allelogram of each SNP.

### Example 4

### Alternative Method of Genotyping Each SNP of the Present Invention

In addition to the method of genotyping described in Example 3, the skilled artisan could determine the genotype of the polymorphisms of the present invention using the below described alternative method. This method is referred to as the “GBS method” herein and may be performed as described in conjunction with the teaches described elsewhere herein.

Briefly, the direct analysis of the sequence of the polymorphisms of the present invention can be accomplished by DNA sequencing of PCR products corresponding to the same. PCR amplicons are designed to be in close proximity to the polymorphisms of the present invention using the Primer3 program. The M13_SEQUENCE1 “TGTAAAACGACGGCCAGT (SEQ ID NO:611)” is prepended to each forward PCR primer (see Table VIII). The M13_SEQUENCE2 “CAGGAAACAGCTATGACC (SEQ ID NO: 612)” is prepended to each reverse PCR primer (see Table VIII). Preferred primers for this genotyping reaction are provided in Table X herein.

PCR amplification and purification are performed essentially the same as described in Example 1 herein.

PCR products are sequenced by the dye-terminator method using the M13_SEQUENCE1 and M13_SEQUENCE2 primers above. The genotype can be determined by analysis of the sequencing results at the polymorphic position.

### Example 5

### Statistical Analysis of the Association Between the Low Statin Response Phenotype and the SNPs of the Present Invention

The association between low statin efficacy responses and the single nucleotide polymorphisms of the present invention may be investigated by applying statistical analysis to the results of the genotyping assays described elsewhere herein. The central hypothesis of this analysis would be that a predisposition to develop such a phenotype may be conferred by specific genomic factors. The analysis would attempt to identify one or more of these factors in DNA samples from index cases and matched control subjects exposed to one or more statins, preferably, not not limited to, pravastatin (disclosed in U.S. Pat. No. 4,346,227), in addition to, or alternatively substituted with one or more of the following: lovastatin, simvastatin, fluvastatin, atorvastatin or cerivastatin.

### Methods

Measures. Single nucleotide polymorphisms (SNPs) in OATP2 and cMOAT gene regions are genotyped on all subjects essentially as described in Example 3 herein.

Statistical Analyses. Conditional logistic regression (HOSMER and LEMESHOW 2000) may be used to examine the associations between genotypes of the SNPs of the present invention and the development of low statin efficacy responses. All SNPs may be bi-allelic with three possible genotypes. For each SNP, in the overall sample and each subgroup, allele frequencies may be estimated. For consistency in SNP genotype parameter coding in the logistic regression models, the less frequent allele of each SNP may be designated as the rare allele and the number of copies of that allele that each subject carried, either 0, 1, or 2, may then be determined. Three possible genotypes for each SNP leaves two degrees of freedom for parameters in the conditional logistic regression model representing the information contained in these three genotype categories. Two dummy variables may be therefore created based on the copies of the rare allele for each subject for use in the conditional logistic regression model,

x₁=1 if copies of rare allele=1, 0 otherwise and

x₂=1 if copies of rare allele=2, 0 otherwise.

The full conditional logistic regression model used was

$${π}_{k} ( x ) = \frac{{ⅇ}^{{α}_{k} + {β}_{1}^{′} {x}_{1} + {β}_{2}^{′} {x}_{2}}}{1 + {ⅇ}^{{α}_{k} + {β}_{1}^{′} {x}_{1} + {β}_{2}^{′} {x}_{2}}} ,$$

where x in πₖ(x) is the vector of dummy variables representing the SNP genotypes described above, k is the matching stratum index specific to each matched case-control set of subjects, πₖ(x) is the matching stratum-specific expected probability that a subject is a case given x, αₖ is the matching stratum-specific contribution to πₖ(x) of all the matching variables constant within the kth stratum and each β′ represents the contribution of the respective dummy variable to πₖ(x).

For each SNP, the null hypothesis is that the vector of β′ are all equal to 0 and are tested using the scores test (HOSMER and LEMESHOW 2000). The degrees of freedom for the scores test statistic may be equal to one less than the number of genotypes. Exponentiation of each slope coefficient, β′, may be used to provide an estimate of the ratio of the odds of an adverse event (low statin efficacy response) in subjects carrying the specified copies of the rare allele represented in the definition of the coefficient, relative to controls matched for nationality, race, gender and starting dose, over the odds of such an adverse event for similarly matched subjects not carrying any copies of the rare allele. 95% confidence interval limits may be estimated for each odds ratio based on the standard error estimate of the respective slope coefficient.

### References

HOSMER, D. W., and S. LEMESHOW, 2000 Applied logistic regression. John Wiley & Sons, New York.

### Example 6

### Method of Isolating the Native Forms of the Organic Anion Transport and Multi-Drug Resistant Genes of the Present Invention

A number of methods have been described in the art that may be utilized in isolating the native forms of the organic anion transport and multi-drug resistant genes. Specific methods for each gene are referenced below and which are hereby incorporated by reference herein in their entireties. The artisan, skilled in the molecular biology arts, would be able to isolate these native forms based upon the methods and information contained, and/or referenced, therein.

### OATP2, Solute Carrier Family 21 Member 6 (HGNC_ID—SLC21A6; Genbank Accession No. —gi|6636521)

1.) Hsiang, B. et al., J. Biol. Chem. 274 (52), 37161-37168 (1999) (hereby incorporated herein by reference in its entirety).

2.) PCT International Publication No: WO0071566 (hereby incorporated herein by reference in its entirety).

### cMOAT, ATP-Binding Cassette Sub-Family C Member 2 (HGNC_ID—ABCC2; Genbank Accession No. —gi|6636521)

1.) Taniguchi, K., Wada, M., Kohno, K., Nakamura, T., Kawabe, T., Kawakami, M., Kagotani, K., Okumura, K., Akiyama, S. and Kuwano, M. Cancer Res. 56 (18), 4124-4129 (1996)

2.) Japanese Patent Application No.: JP10099079 (hereby incorporated herein by reference in its entirety).

### Example 7

### Method of Isolating the Novel Polymorphic Forms of the Organic Anion Transport and Multi-Drug Resistant Genes of the Present Invention

Since the novel allelic genes of the present invention represent genes present within at least a subset of the human population, these genes may be isolated using the methods provided in Example 3 above. For example, the source DNA used to isolate the novel allelic gene may be obtained through a random sampling of the human population and repeated until the allelic form of the gene is obtained. Preferably, random samples of source DNA from the human population are screened using the SNPs and methods of the present invention to identify those sources that comprise the allelic form of the gene. Once identified, such a source may be used to isolate the allelic form of the gene(s). The invention encompasses the isolation of such allelic genes from both genomic and/or cDNA libraries created from such source(s).

In reference to the specific methods provided in Example 3 above, it is expected that isolating the organic anion transport genes would be within the skill of an artisan trained in the molecular biology arts. Nonetheless, a detailed exemplary method of isolating at least one of the organic anion transporter genes, in this case the variant form (P155T) of OATP2, solute carrier family 21 member 6 cDNA (SNP_ID=PS100s2) is provided. Briefly,

First, the individuals with the P155T variation are identified by genotyping the genomic DNA samples using the FP-SBE (Chen X 1999) method, described in Example 1 and 2 above. DNA samples publicly available from the Coriell Institute (Collingswood, N.J.) are used (e.g., the Coriell Sample IDs provided in Table VII herein). Oligonucleotide primers that were used for this genotyping assay are as follows.

| | | | |
|---|---|---|---|
| OATP2.L: | 5′-1TCAGATGGACAAAAThFGCA-3′ | (PCR forward primer) | (SEQ ID NO:613) |
| OATP2.R: | 5′-AAAACACATGCTGGGAAATTG-3′ | (PCR reverse primer) | (SEQ ID NO:614) |
| OATP2: | 5′-cagtaatttatgtctttgtgggcc-3′ | (SBE primer) | (SEQ ID NO:615) |

By analyzing the genomic DNA samples (Coriell), individuals may be identified that harbor the P155T form of the OATP2receptor. Next, Lymphoblastoid cell lines from these individuals may be obtained from the Coriell Institute. These cells can be grown in RPMI-1640 medium with L-glutamine plus 10% FCS at 37 degrees. PolyA+RNA are then isolated from these cells using Oligotex Direct Kit (Life Technologies).

First strand cDNA (complementary DNA) is produced using Superscript Preamplification System for First Strand cDNA Synthesis (Life Technologies, Cat No 18089-011) using these polyA+RNA as templates, as specified in the users manual which is hereby incorporated herein by reference in its entirety. Specific cDNA encoding OATP2receptor is amplified by polymerase chain reaction (PCR) using a forward primer which hybridizes to the 5′-UTR region, a reverse primer which hybridizes to the 3′-UTR region, and these first strand cDNA as templates (Sambrook, Fritsch et al. 1989). For example, the primers specified in Tables VIII and IX may be used. Alternatively, these primers may be designed using Primer3 program (Rozen S 2000). Restriction enzyme sites (example: SalI for the forward primer, and NotI for reverse primer) are added to the 5′-end of these primer sequences to facilitate cloning into expression vectors after PCR amplification. PCR amplification may be performed essentially as described in the owner's manual of the Expand Long Template PCR System (Roche Molecular Biochemicals) following manufacturer's standard protocol, which is hereby incorporated herein by reference in its entirety.

PCR amplification products are digested with restriction enzymes (such as SalI and NotI, for example) and ligated with expression vector DNA cut with the same set of restriction enzymes. pSPORT (Invitrogen) is one example of such an expression vector. After ligated DNA is introduced into E. coli cells (Sambrook, Fritsch et al. 1989), plasmid DNA is isolated from these bacterial cells. This plasmid DNA is sequenced to confirm the presence an intact (full-length) coding region of the human OATP2 receptor with P155T variation using methods well known in the art and described elsewhere herein.

The skilled artisan would appreciate that the above metod may be applied to isolating the other novel polymorphic bradykinin associated genes of the present invention through the simple subsitution of applicable PCR and sequencing primers. Such primers may be selected from any one of the applicable primers provided in Tables VIII and/or IX, or may be designed using the Primer3 program (Rozen S 2000) as described. Such primers may preferably comprise at least a portion of any one of the polynucleotide sequences of the present invention.

### Example 8

### Method of Engineering the Novel Forms of the Organic Anion Transport and Multi-Drug Resistant Genes of the Present Invention

Aside from isolating the novel allelic genes of the present invention from DNA samples obtained from the human population and/or the Coriell Institute, as described in Example 4 above, the invention also encompasses methods of engineering the novel allelic genes of the present invention through the application of site-directed mutagenesis to the isolated native forms of the genes. Such methodology could be applied to synthesize allelic forms of the genes comprising at least one, or more, of the encoding SNPs of the present invention (e.g., silent, missense)—preferably at least 1, 2, 3, or 4 encoding SNPs for each gene.

In reference to the specific methods provided in Example 4 above, it is expected that isolating the novel polymorphic organic anion transport genes of the present invention would be within the skill of an artisan trained in the molecular biology arts. Nonetheless, a detailed exemplary method of engineering at least one of the organic anion transport genes to comprise the encoding and/or non-coding polymorphic nucleic acid sequence, in this case the variant form (P155T) of OATP2 cDNA (SNP_ID=PS100s2) is provided. Briefly,

cDNA clones encoding the human OATP2 may be identified by homology searches with the BLASTN program (Altschul S F 1990) against the Genbank non-redundant nucleotide sequence database using the published human OATP2 cDNA sequence (GenBank Accession No.:gi|6636521). Examples of publicly available human OATP2 cDNA clones discovered in this search may then be identified and obtained. After obtaining these clones, they are sequenced to confirm the validity of the DNA sequences. Alternatively, the OATP2 cDNA may be obtained by refering to the methods taught in the references for OATP2 provided herein.

Once these clones are confirmed to contain the intact wild type cDNA sequence of OATP2 coding region, the P155T polymorphism (mutation) may be introduced into the native sequence using PCR directed in vitro mutagenesis (Cormack 2000). In this method, synthetic oligonucleotides are designed to incorporate a point mutation at one end of an amplified fragment. Following PCR, the amplified fragments are made blunt-ended by treatment with Klenow Fragment.

These fragments are then ligated and subcloned into a vector to facilitate sequence analysis. This method consists of the following steps.

1. Subcloning of cDNA insert into a high copy plasmid vector containing multiple cloning sites and M13 flanking sequences, such as pUC19 (Sambrook, Fritsch et al. 1989), in the forward orientation. The skilled artisan would appreciate that other plasmids could be equally substituted, and may be desirable in certain circumstances.

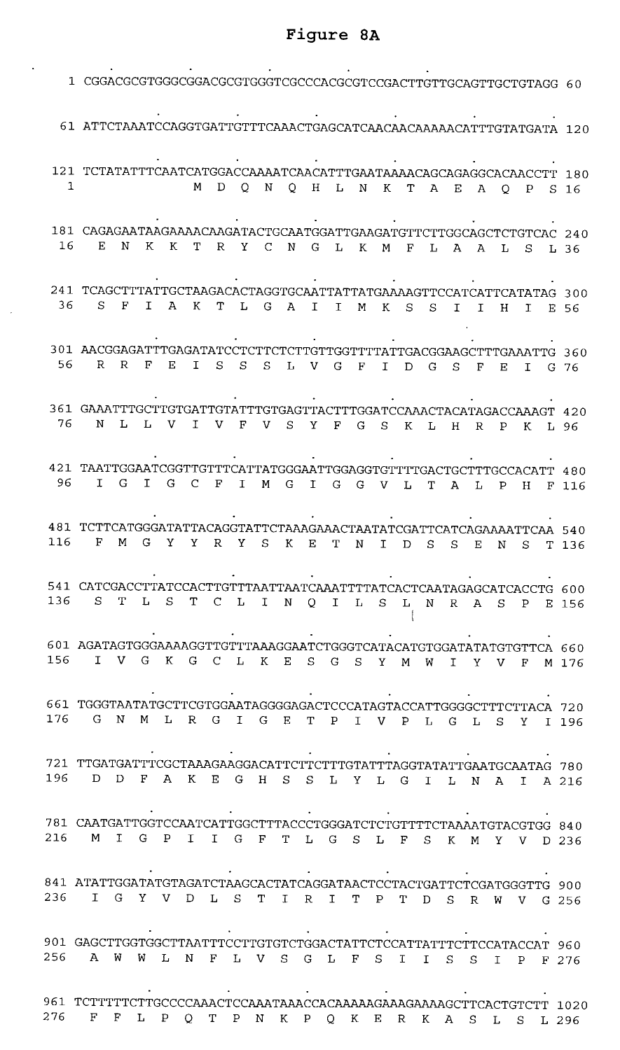

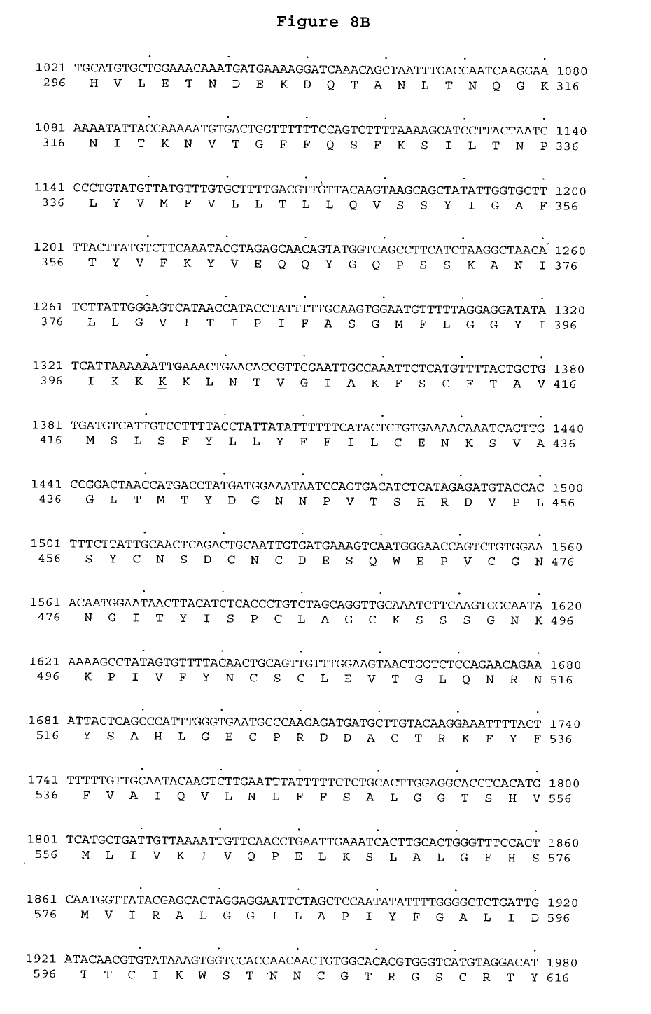

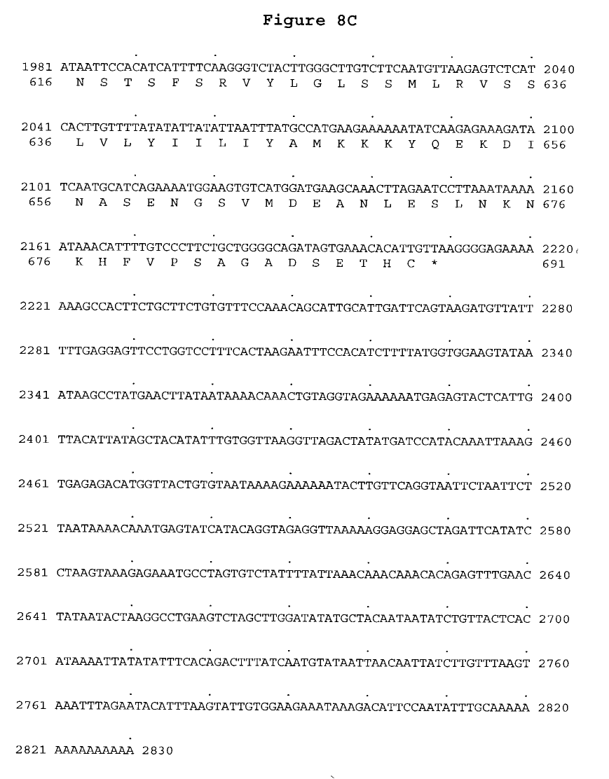

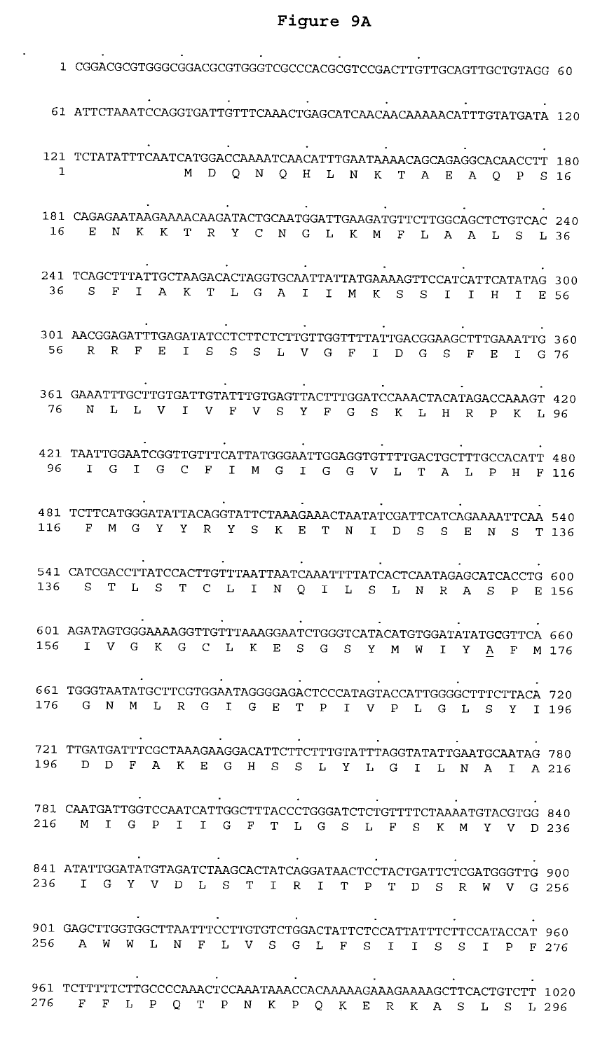

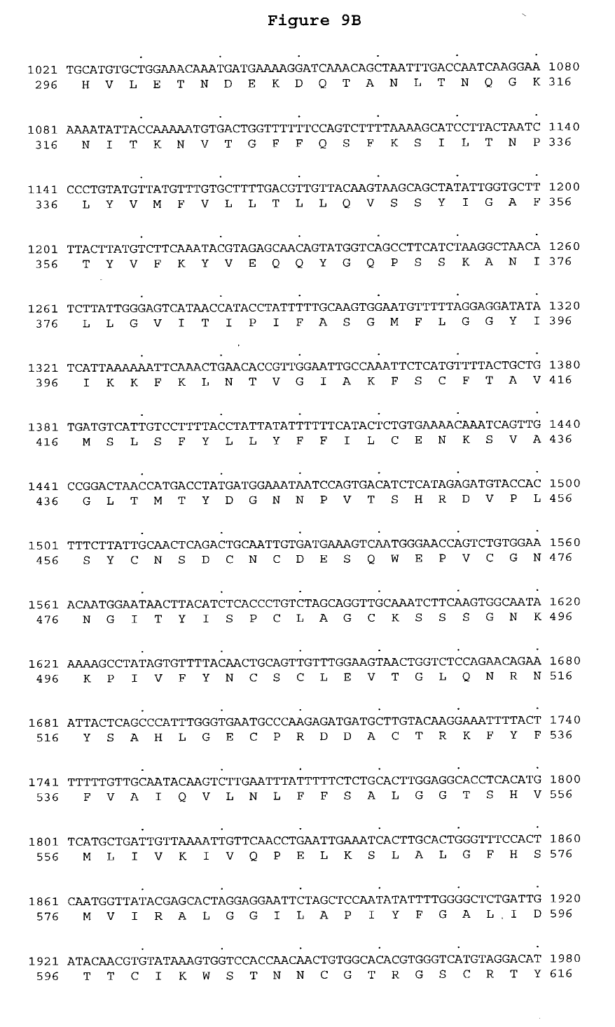

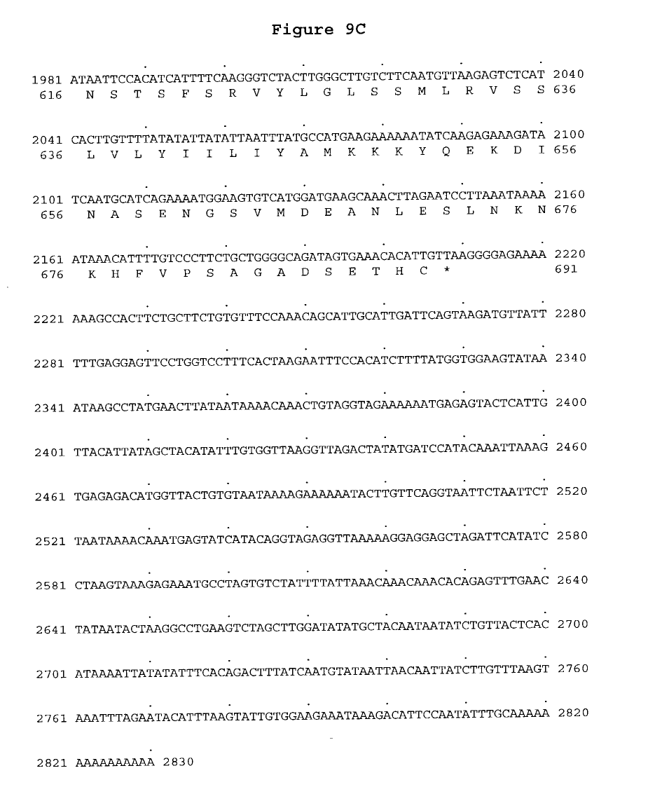

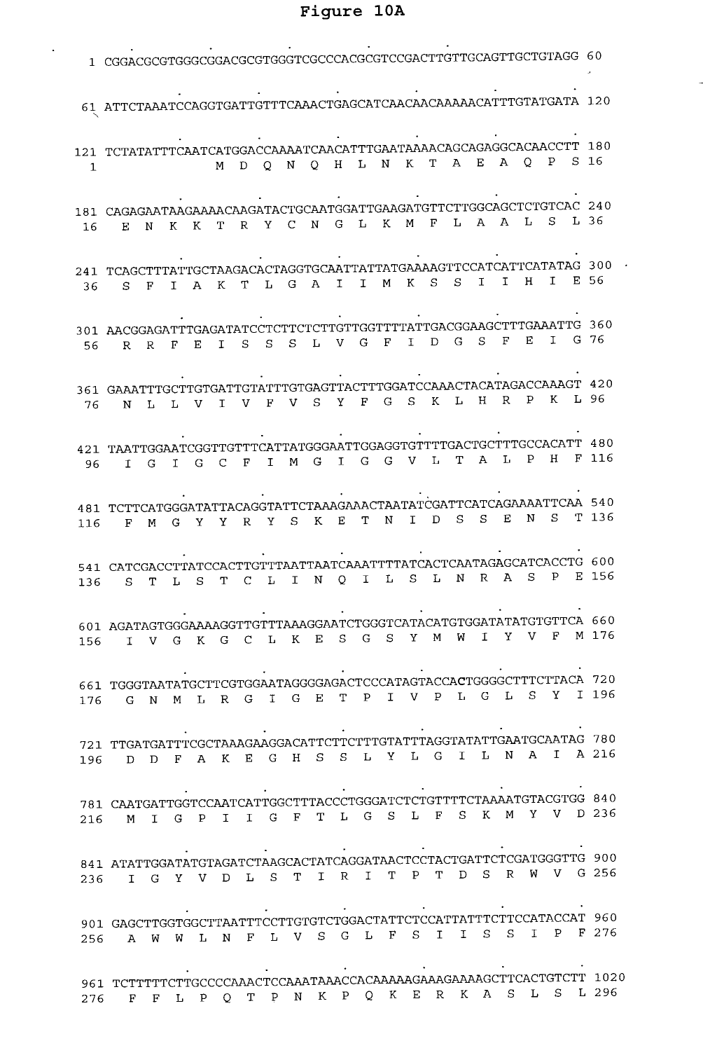

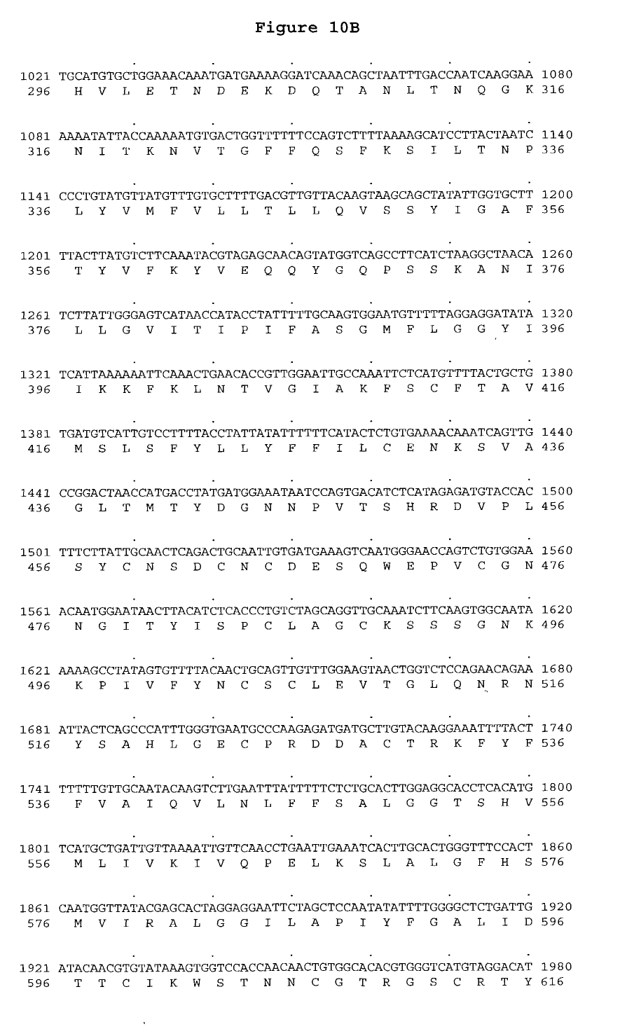

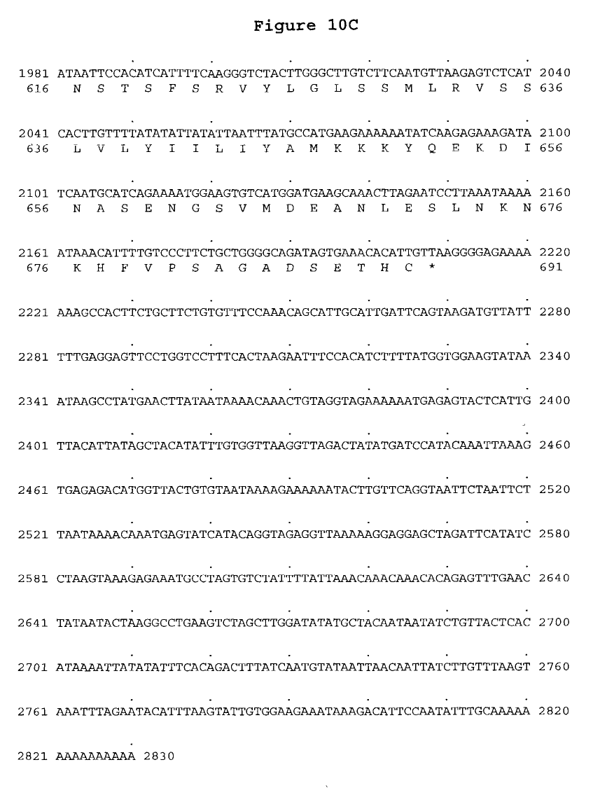

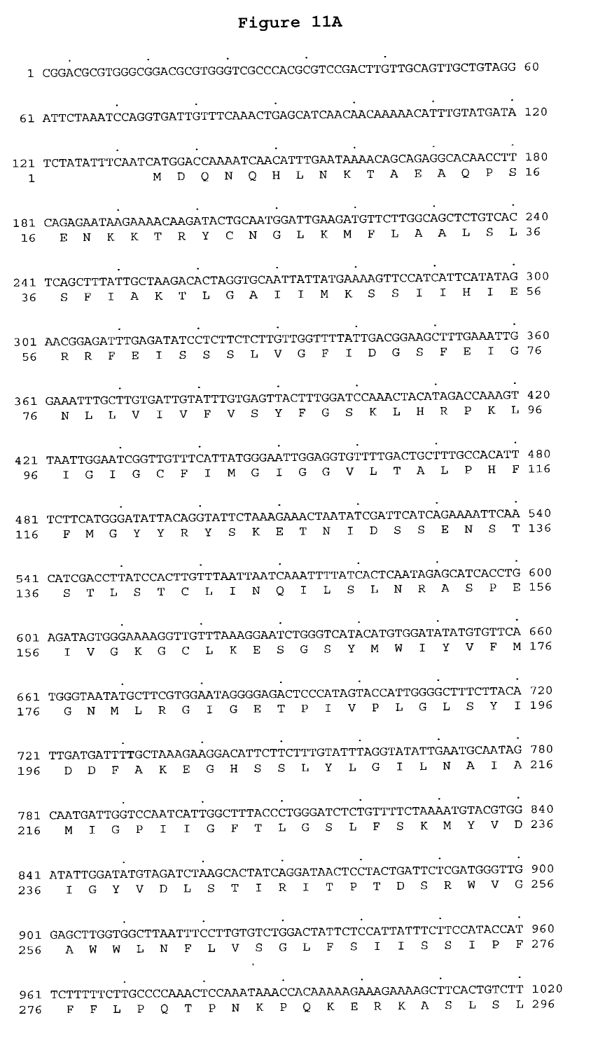

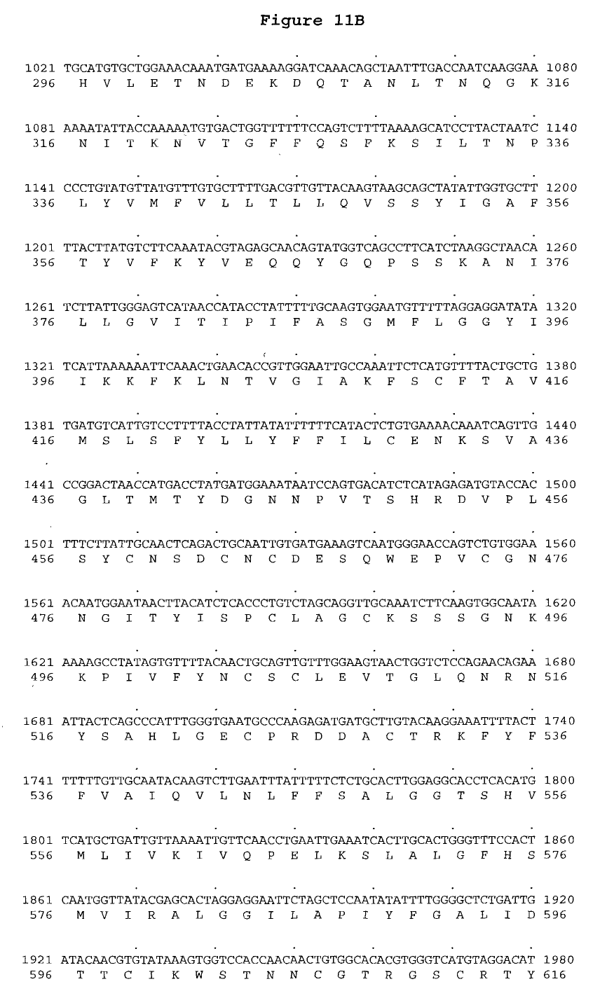

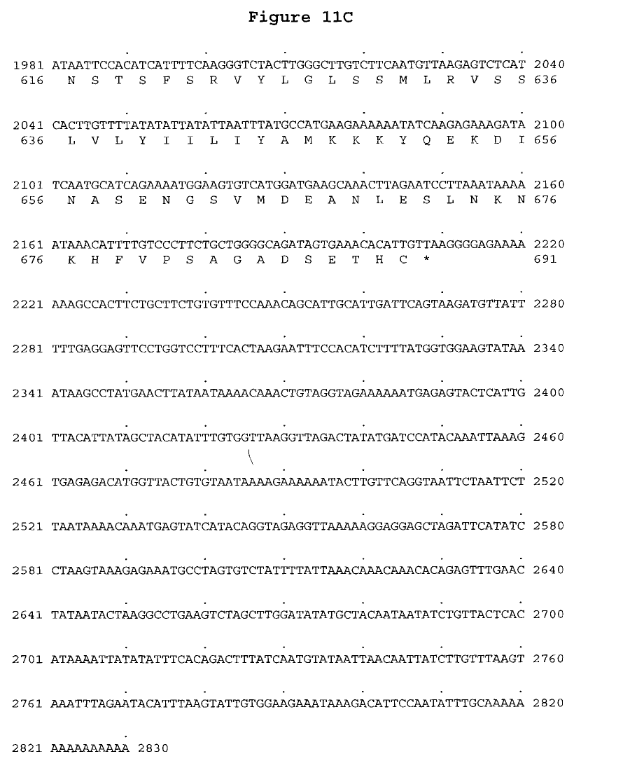

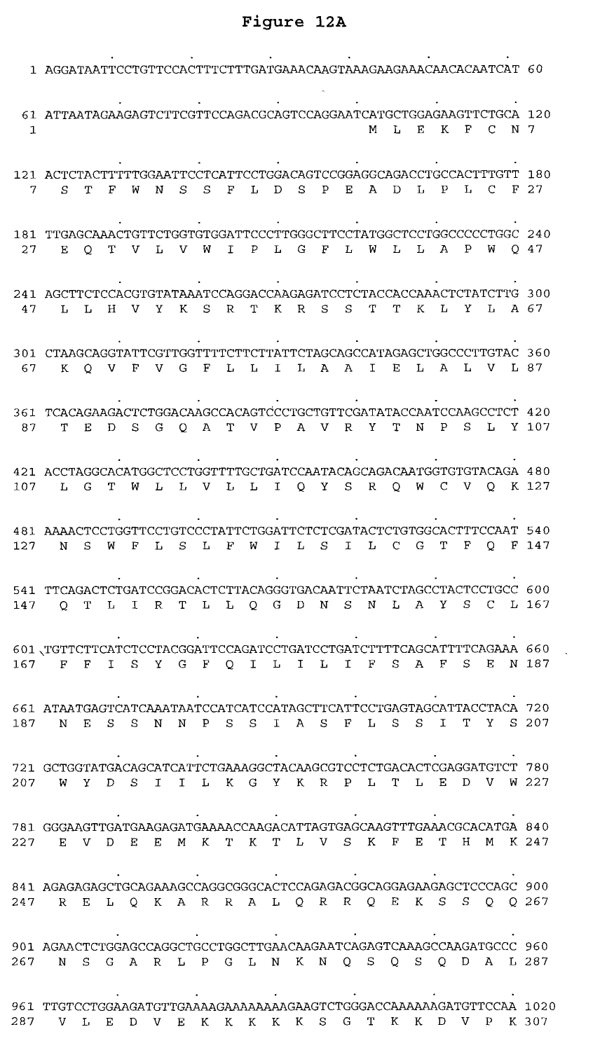

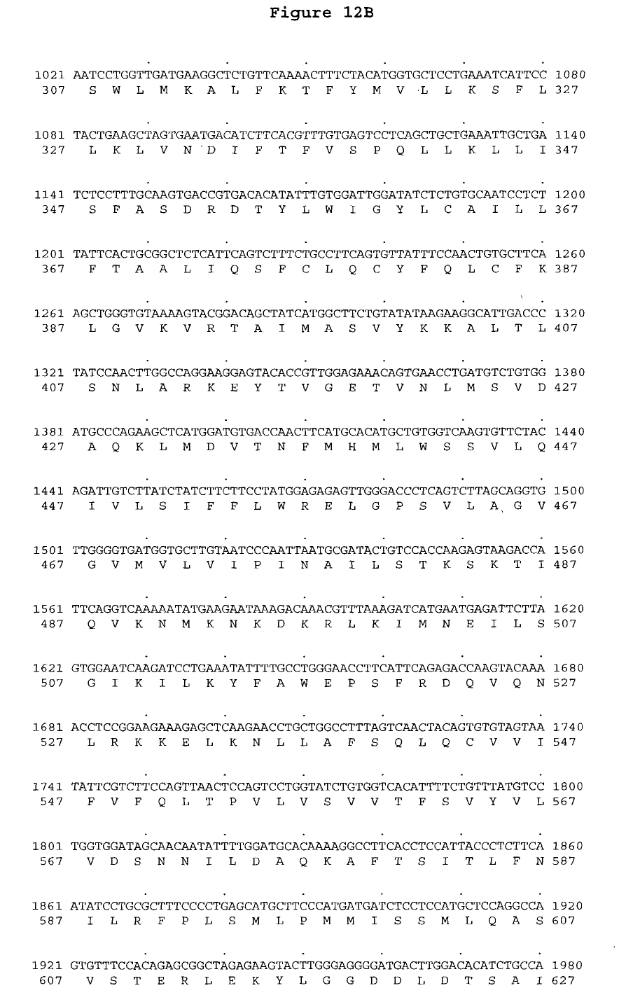

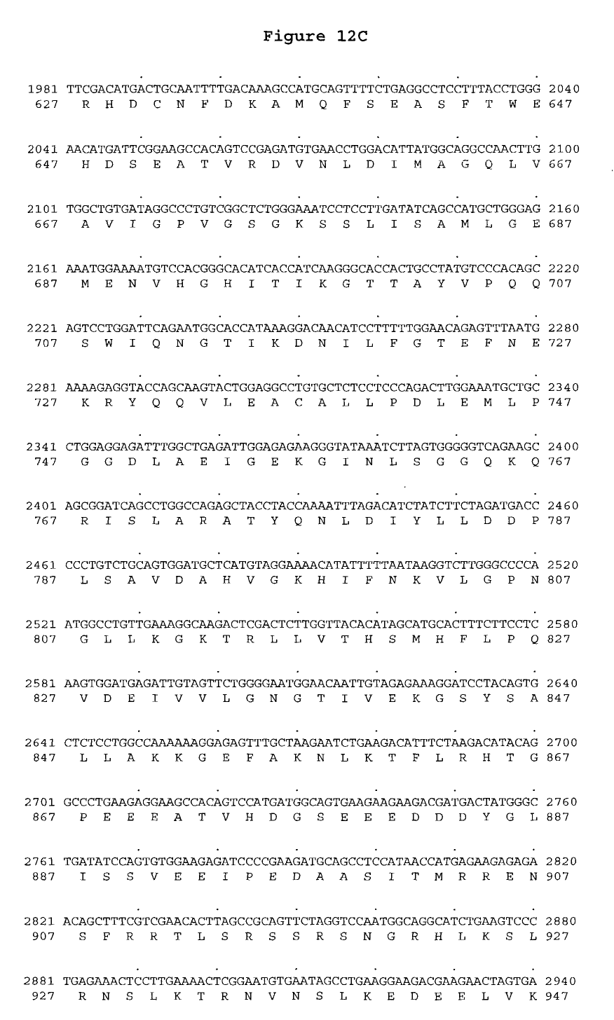

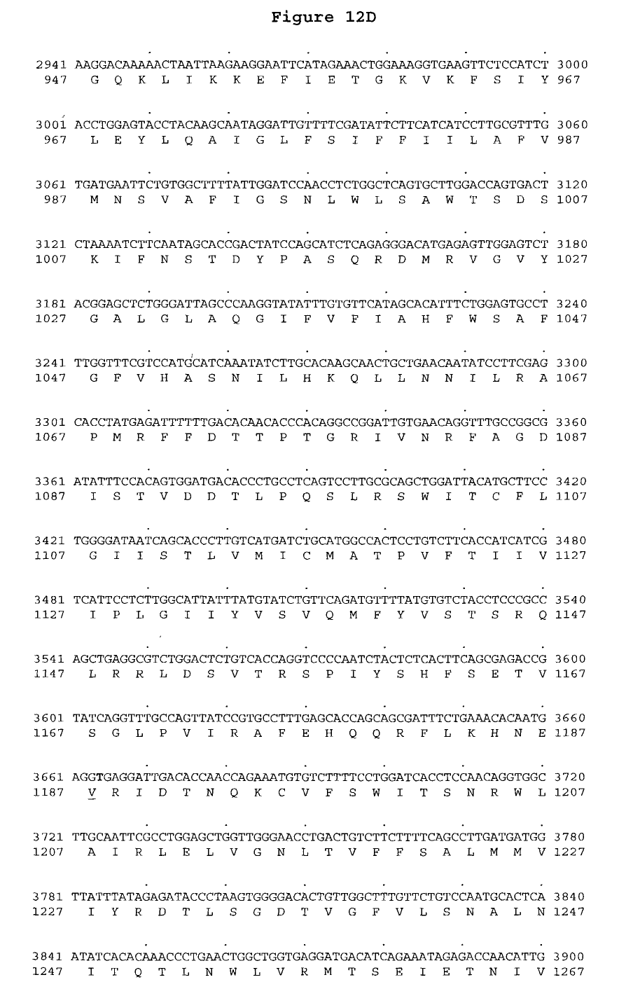

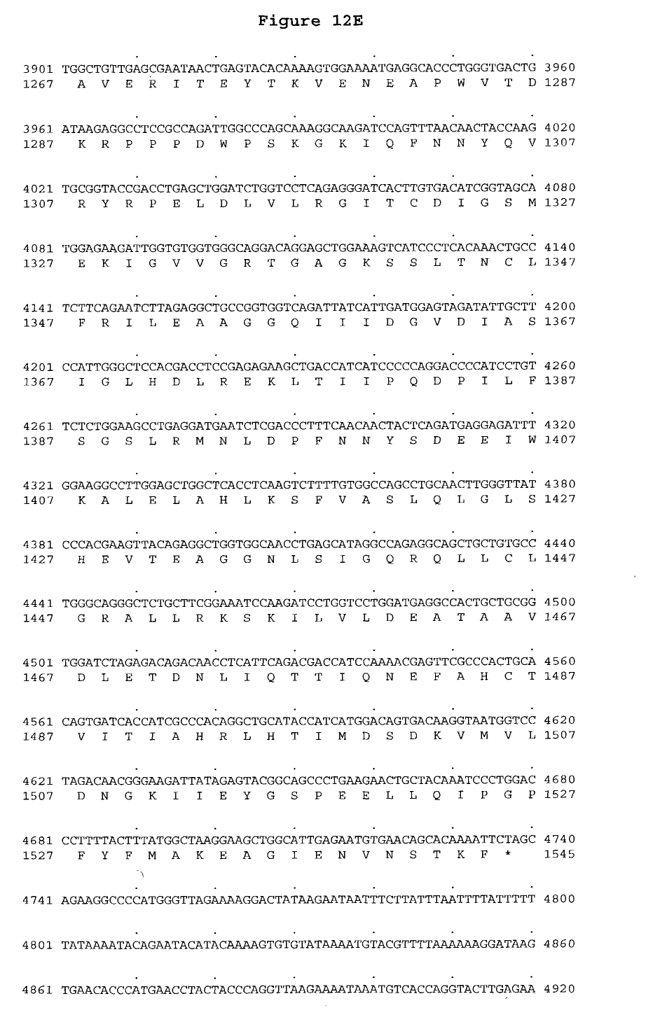

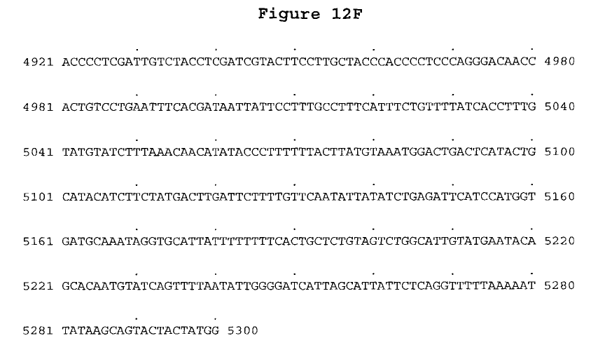

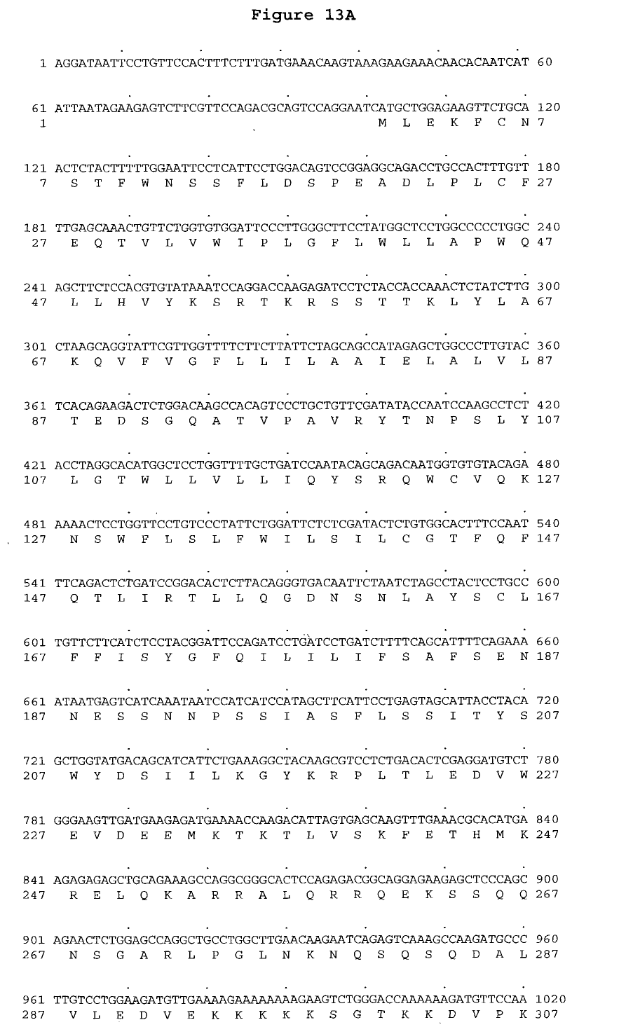

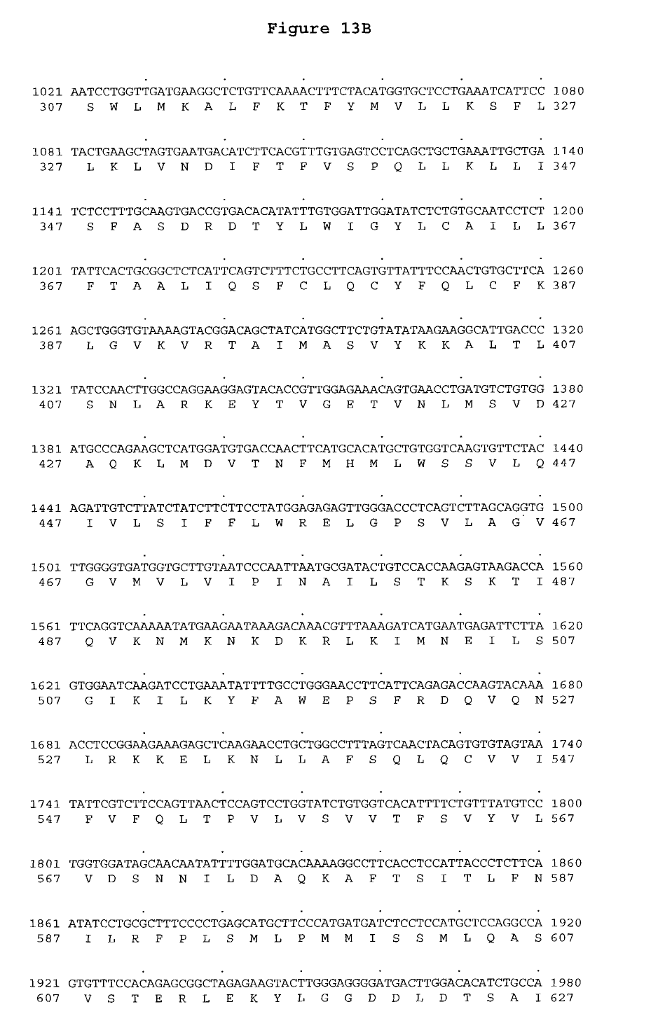

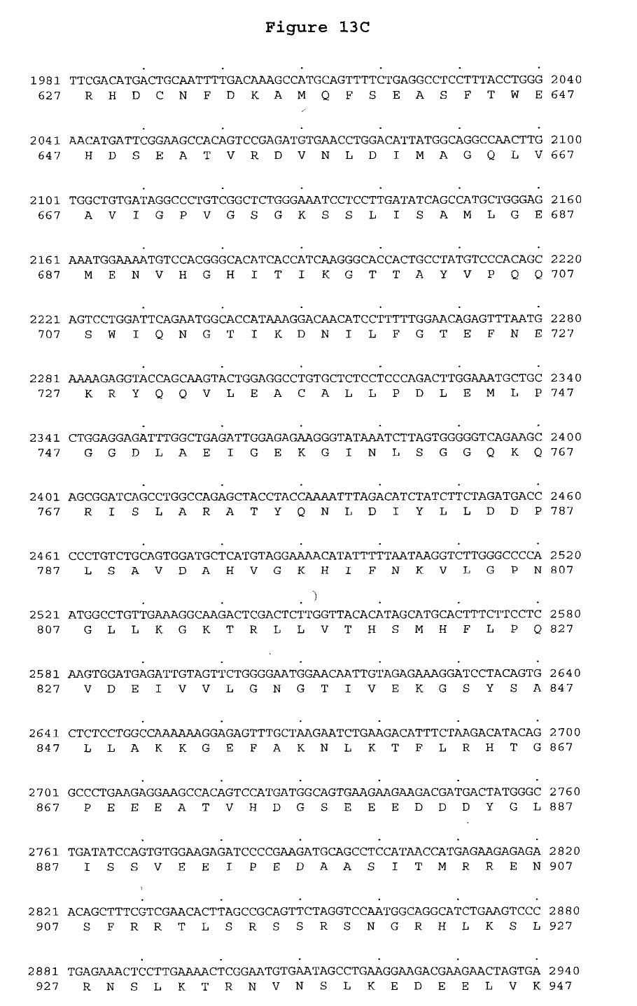

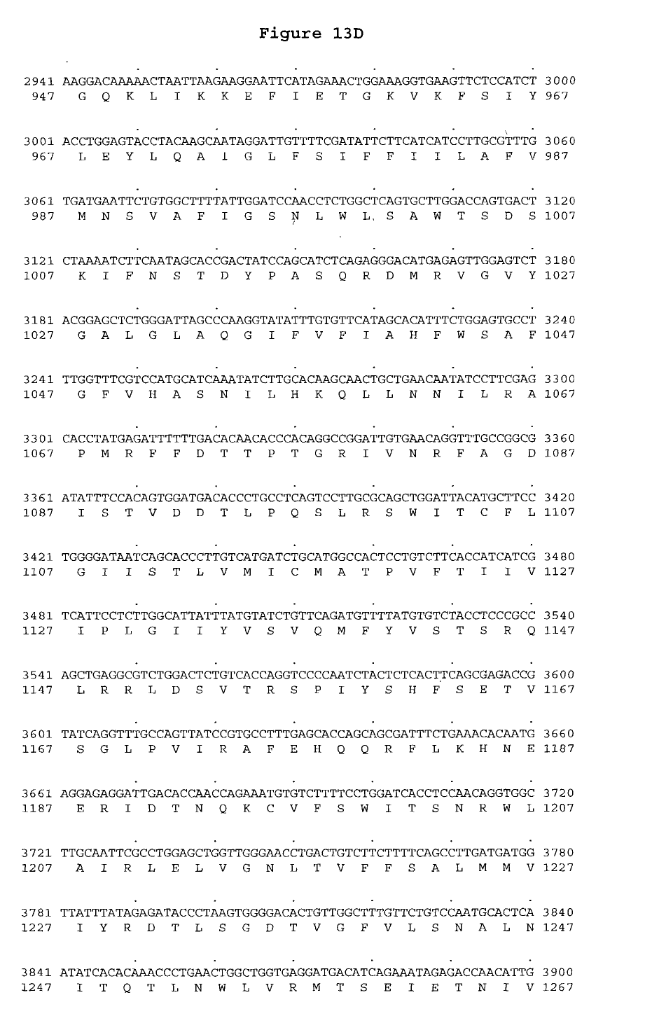

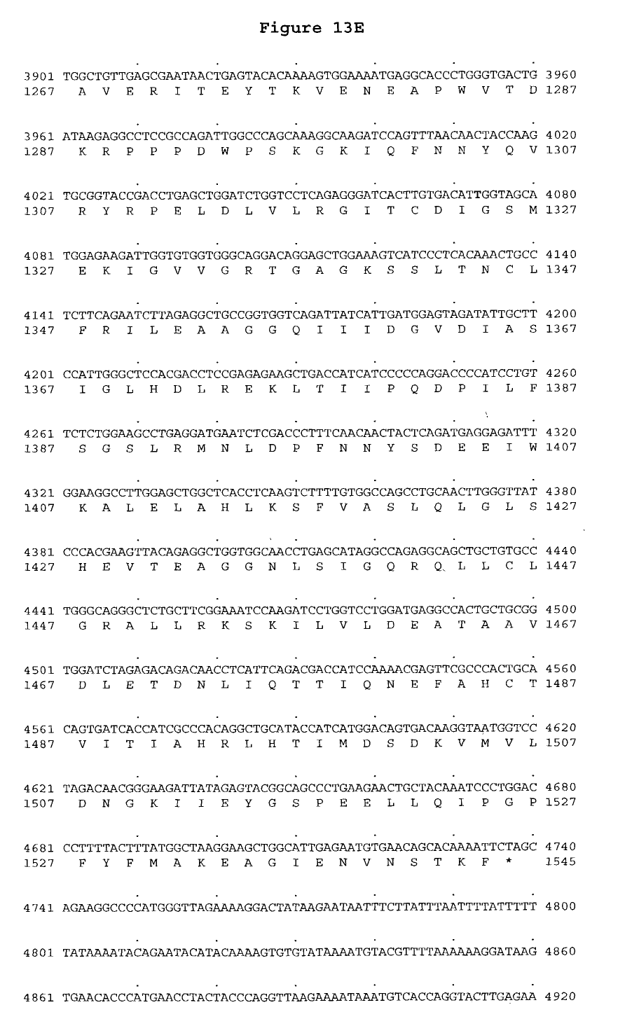

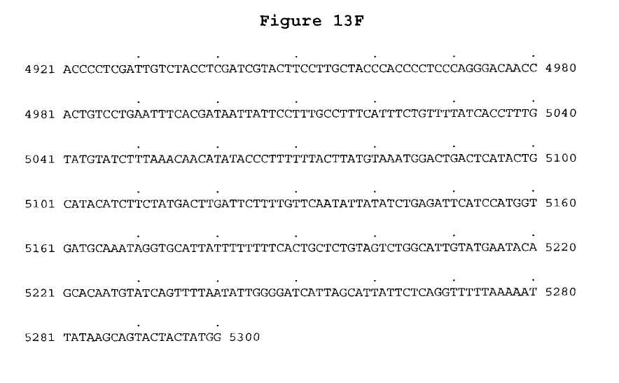

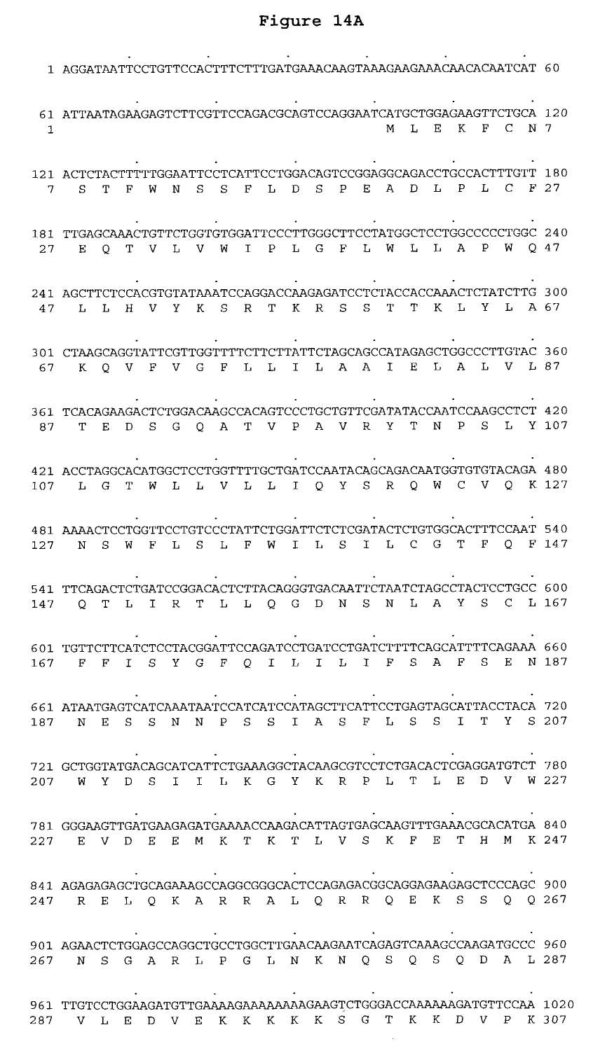

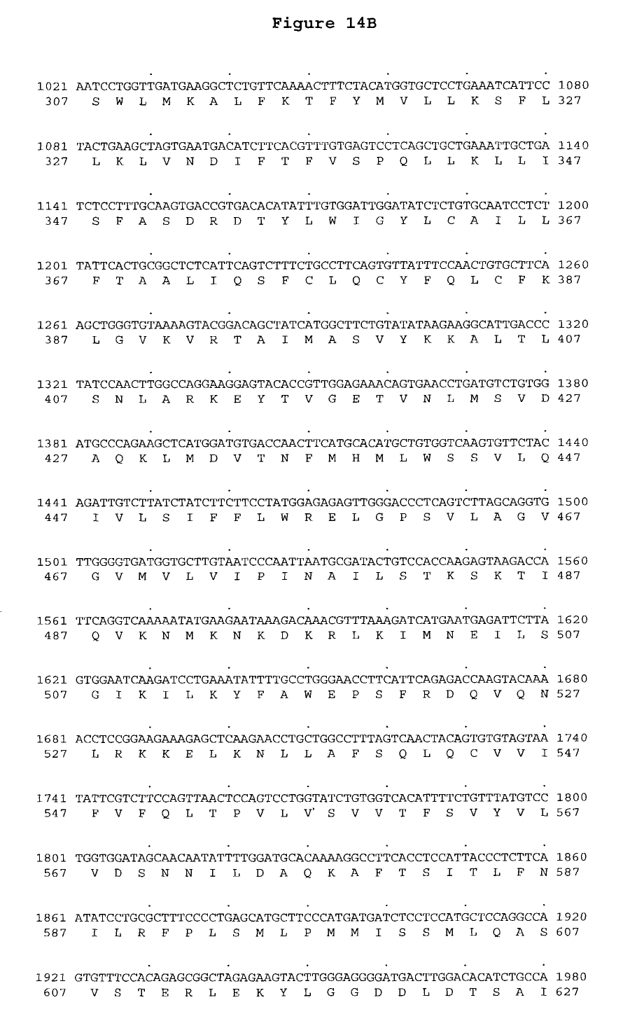

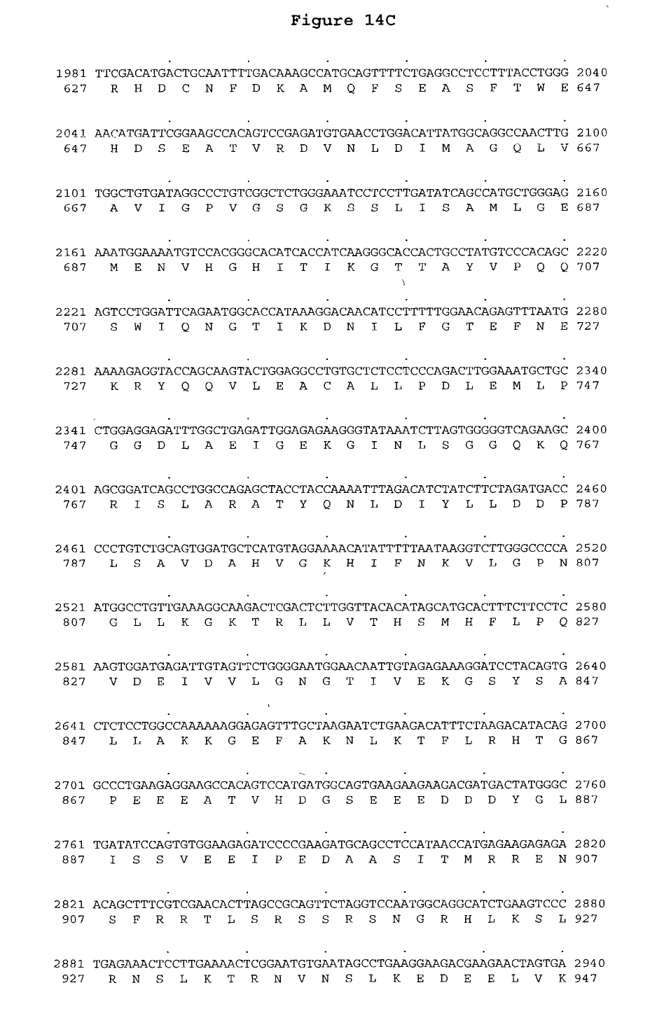

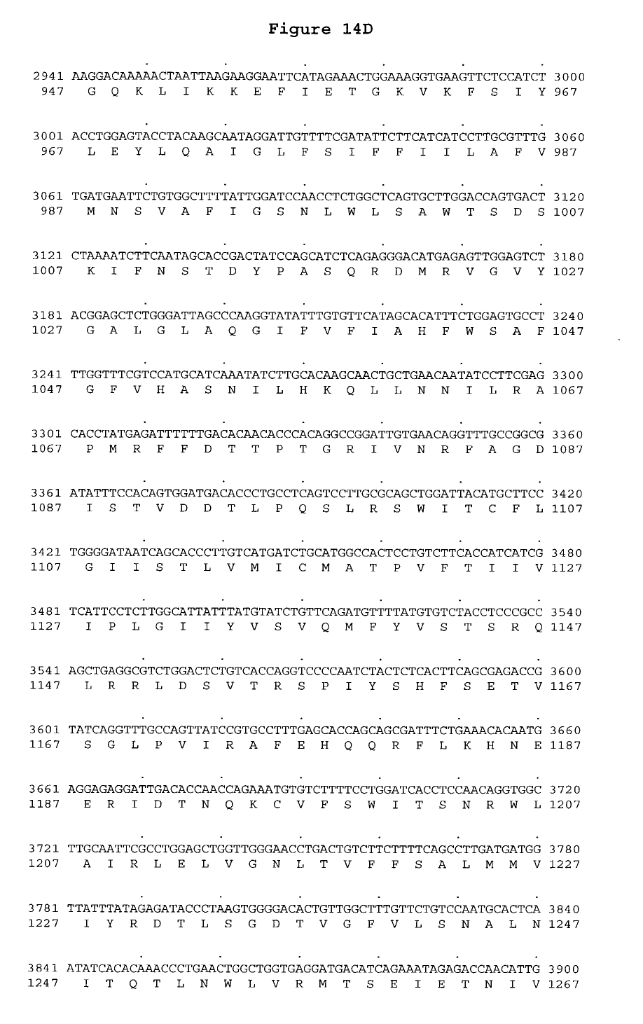

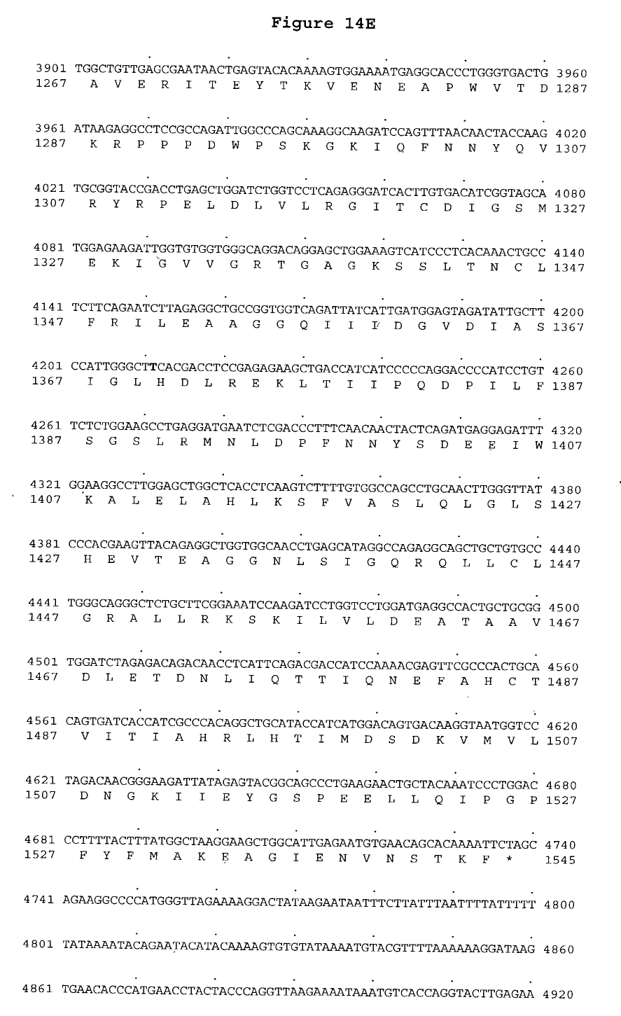

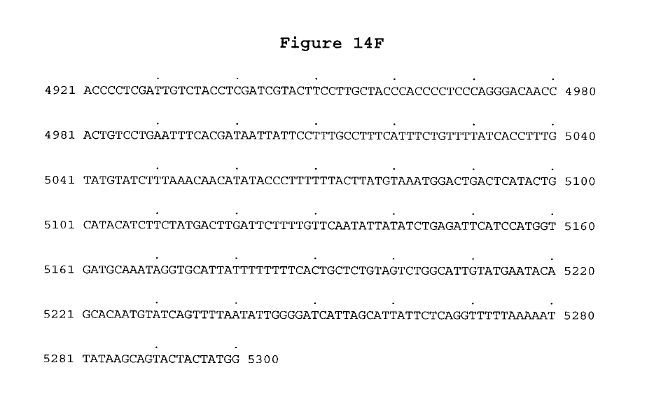

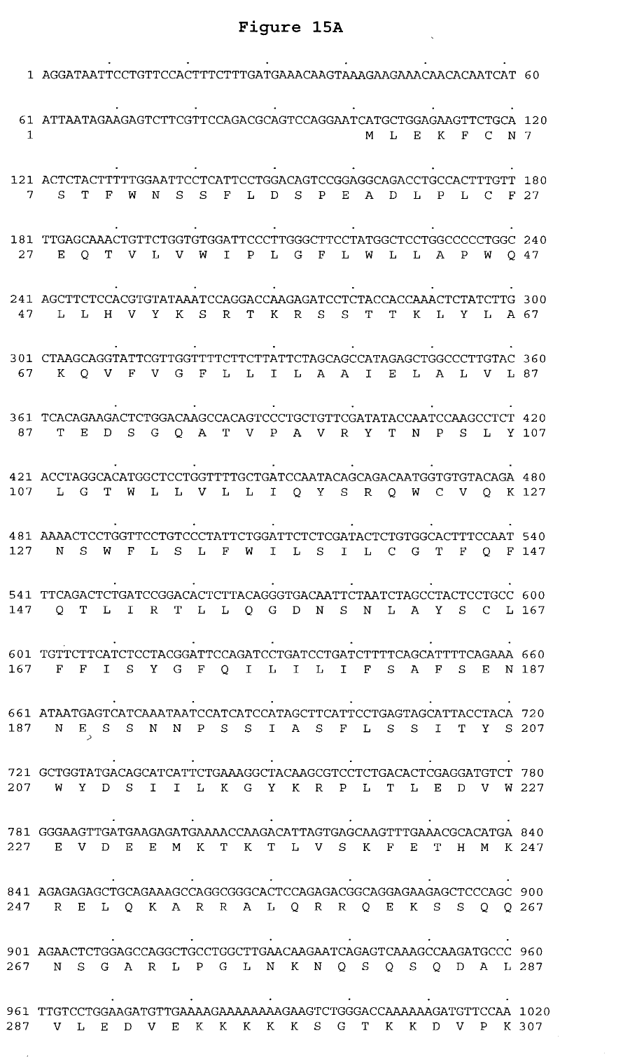

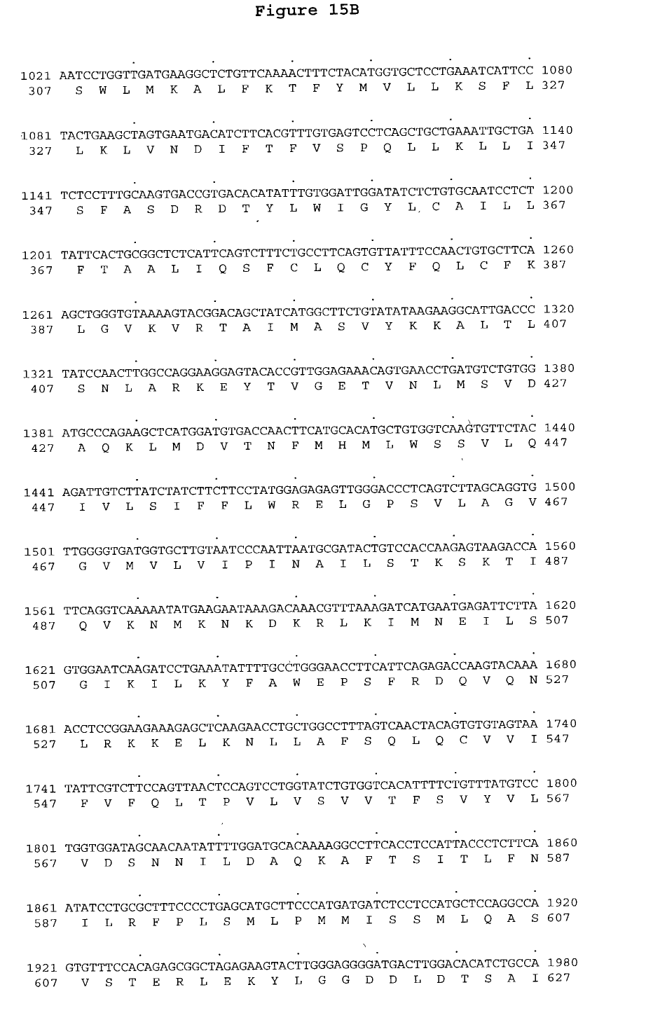

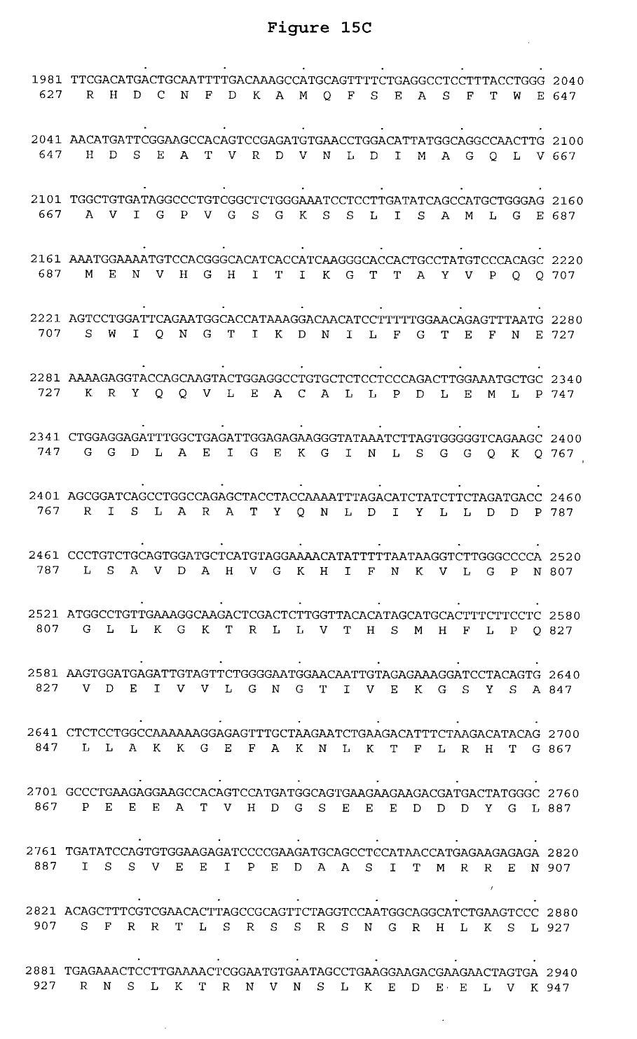

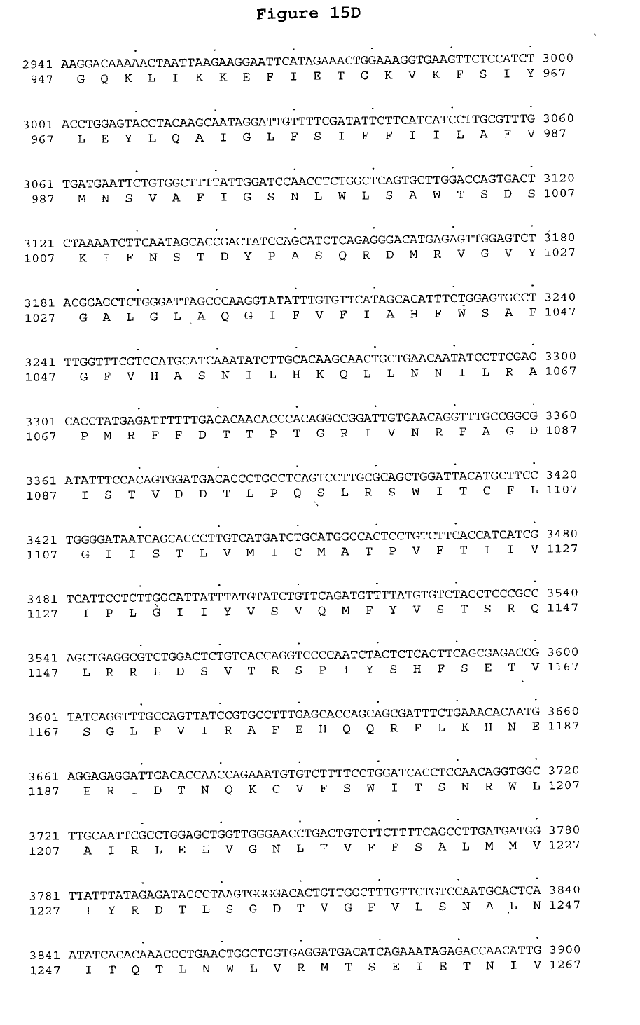

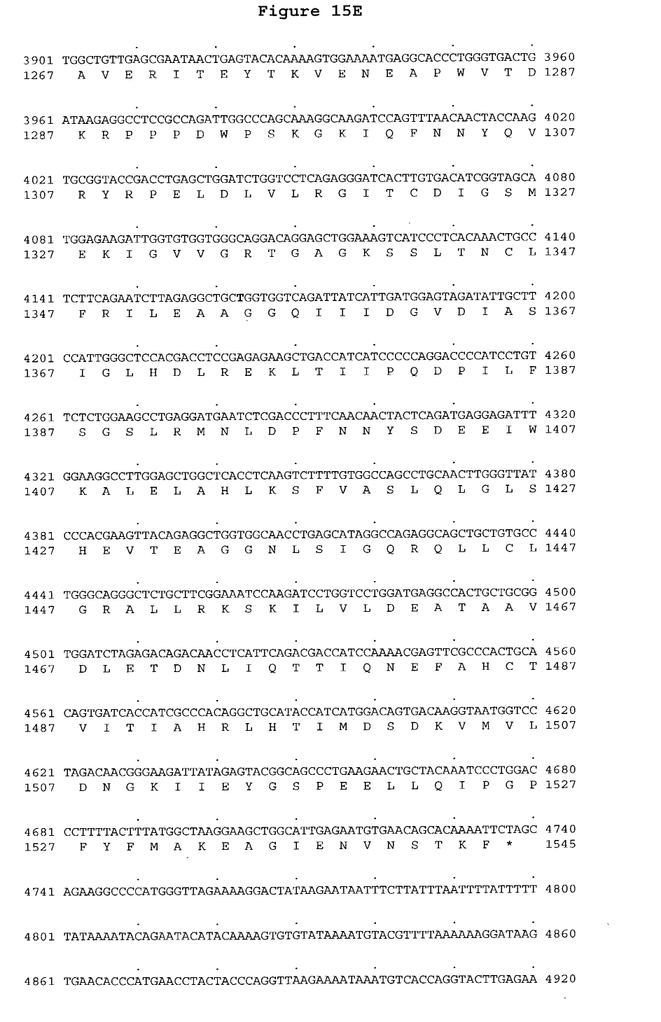

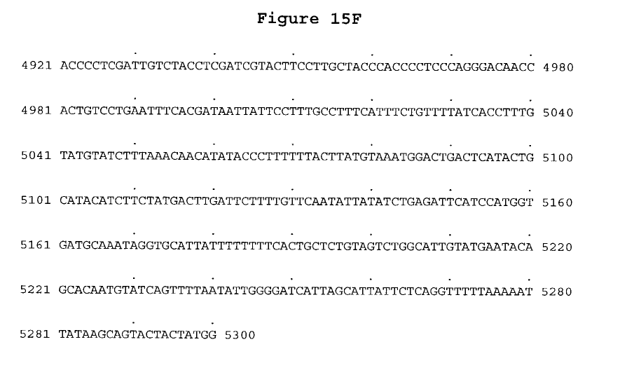

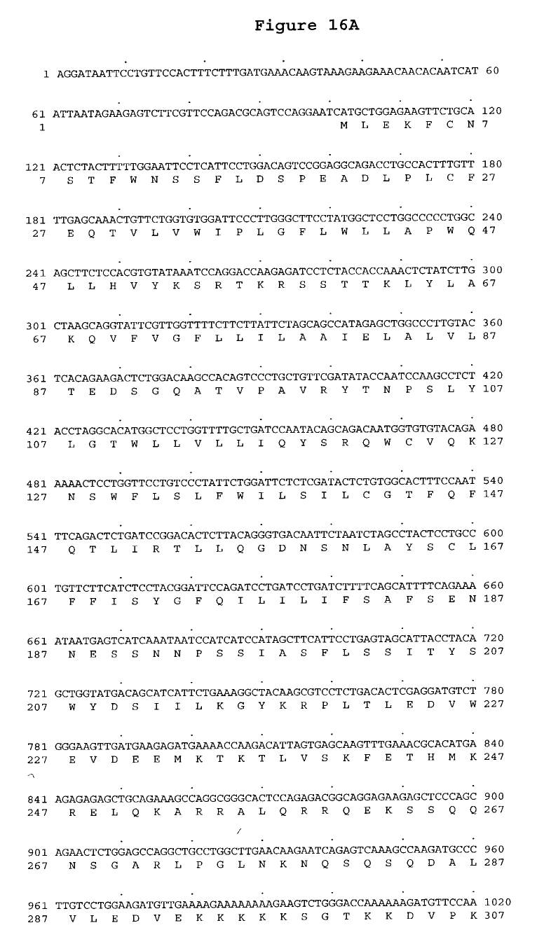

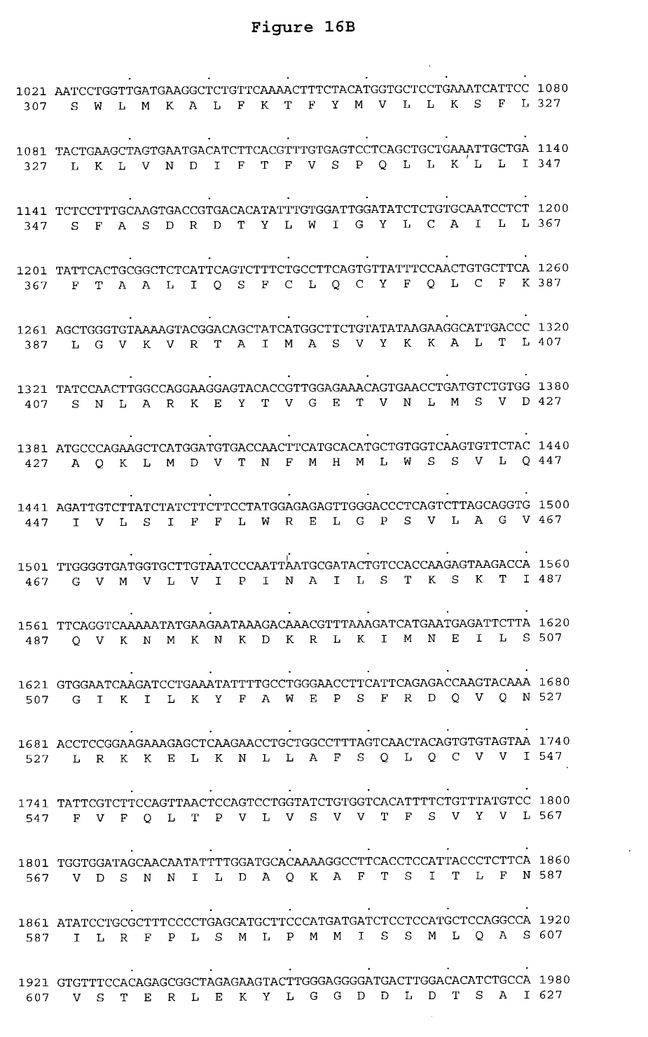

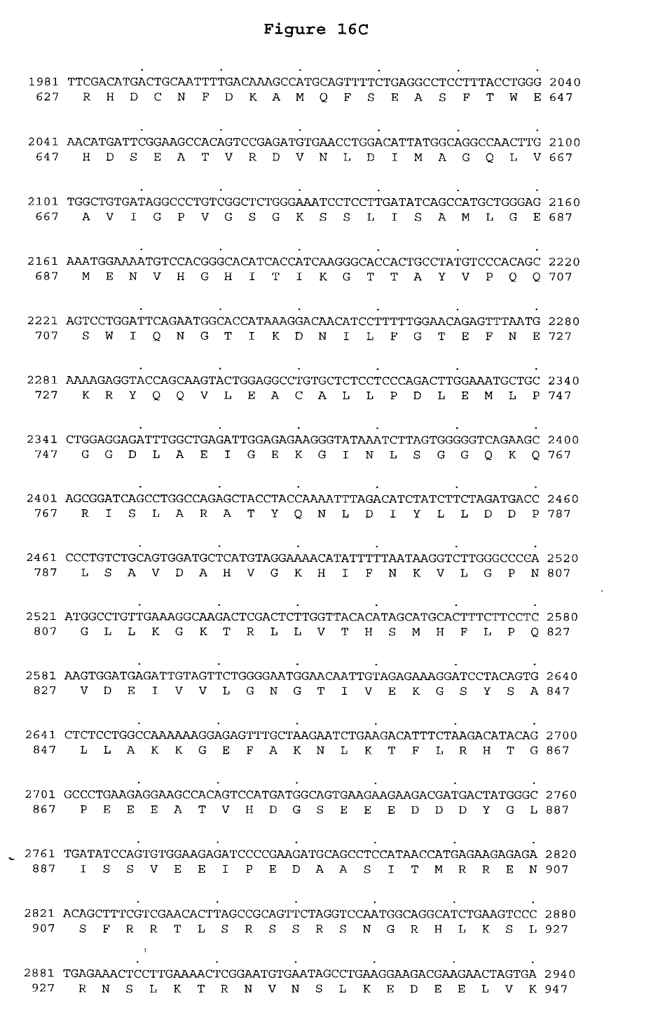

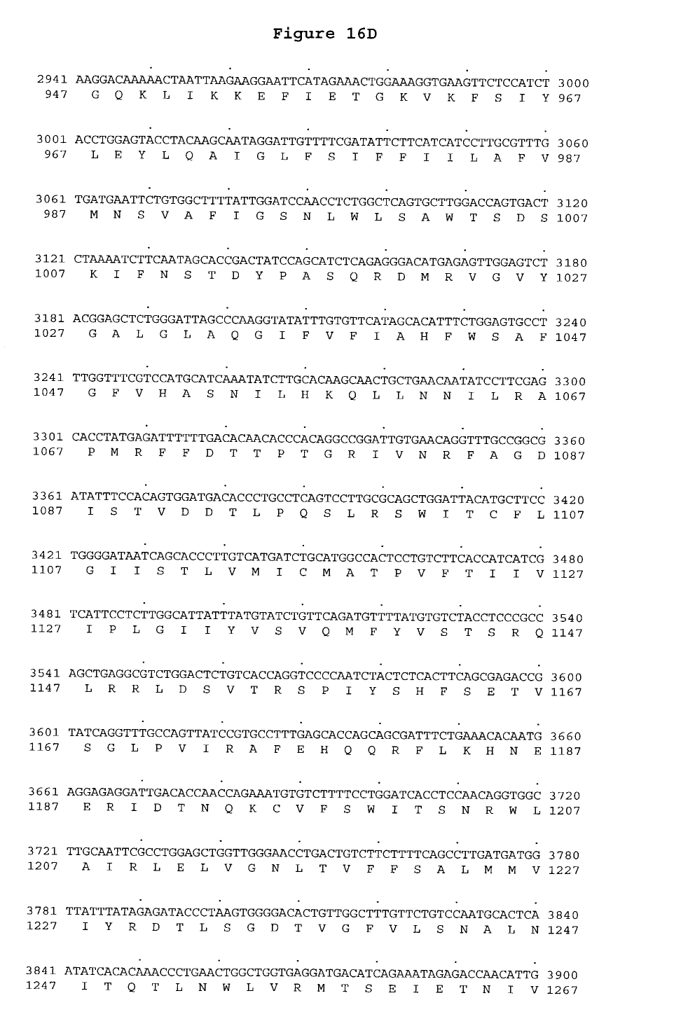

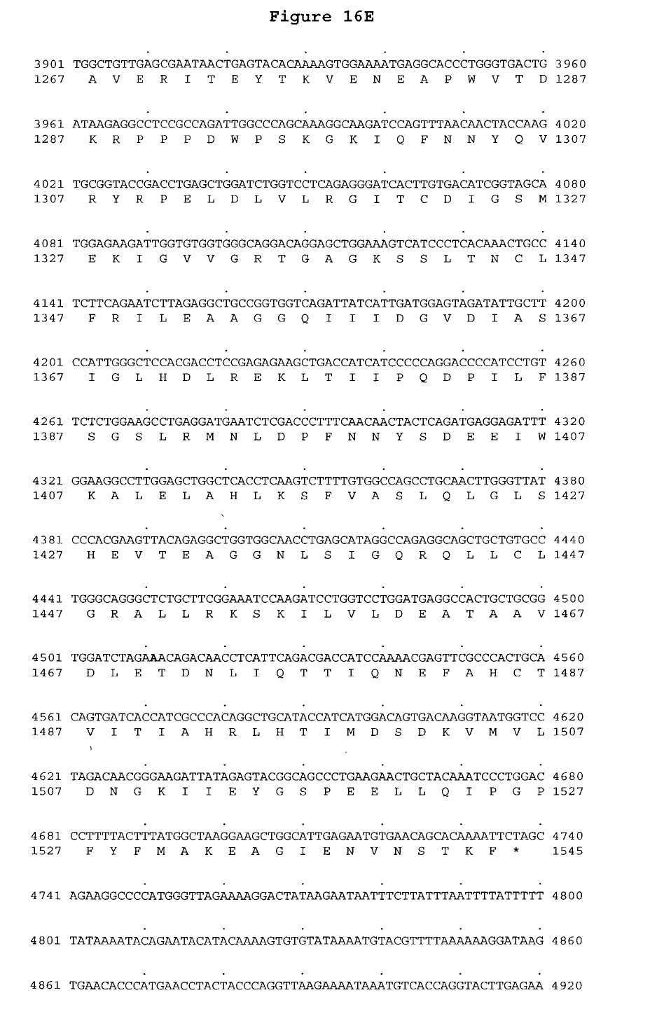

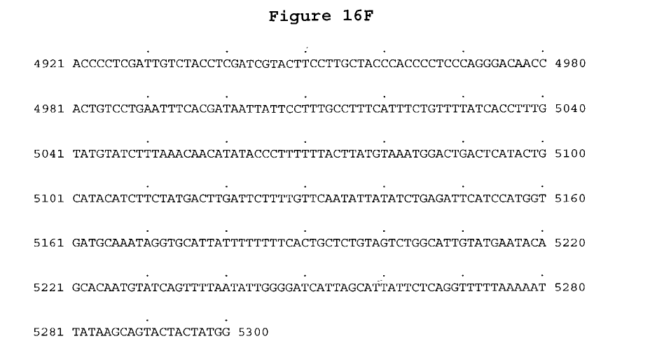

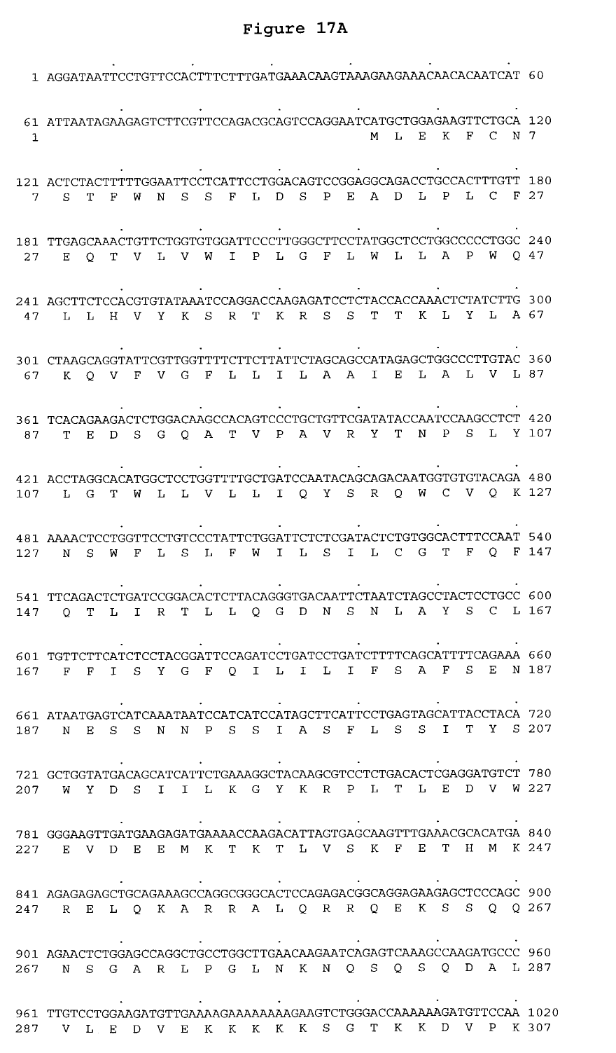

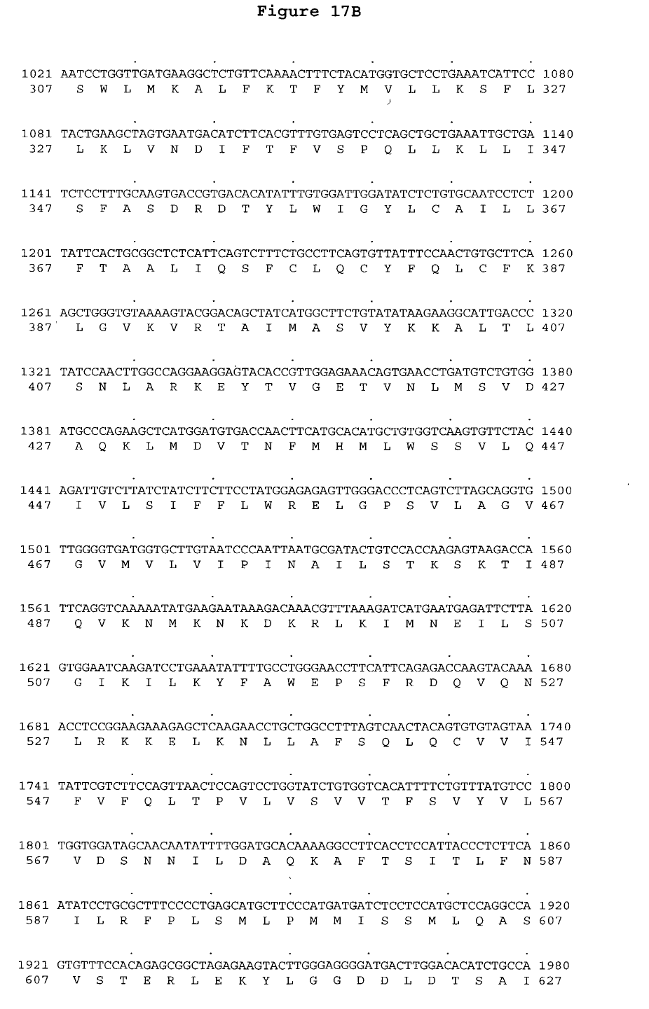

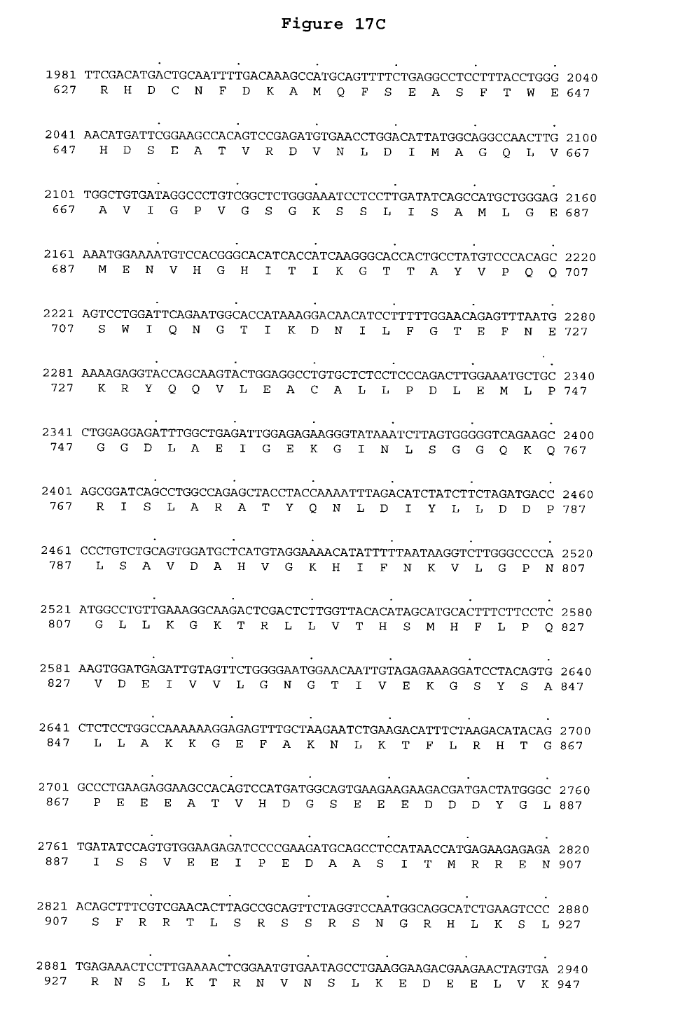

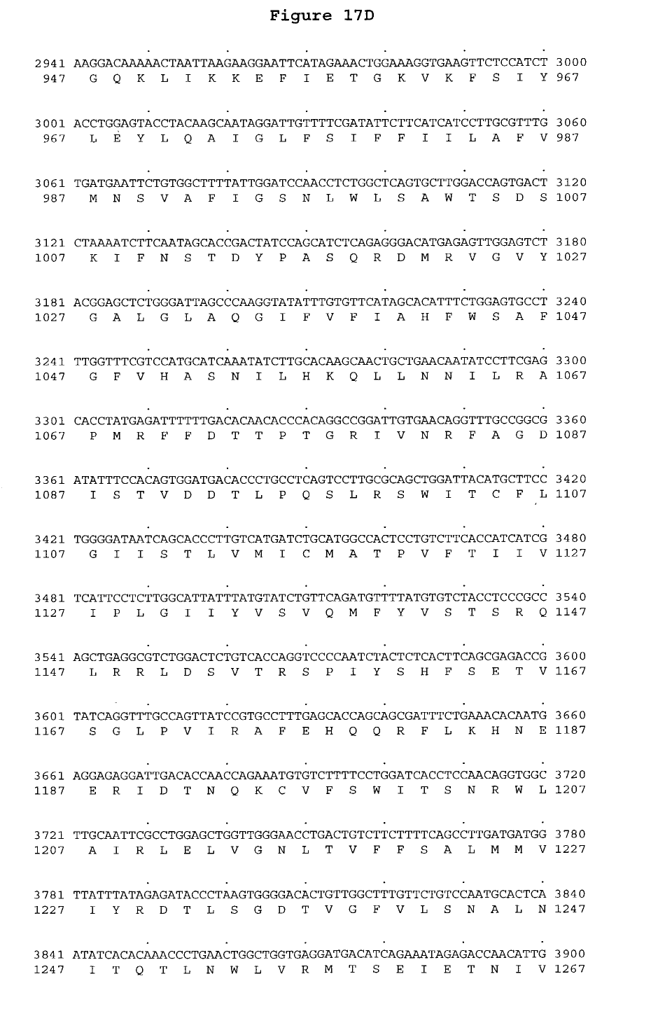

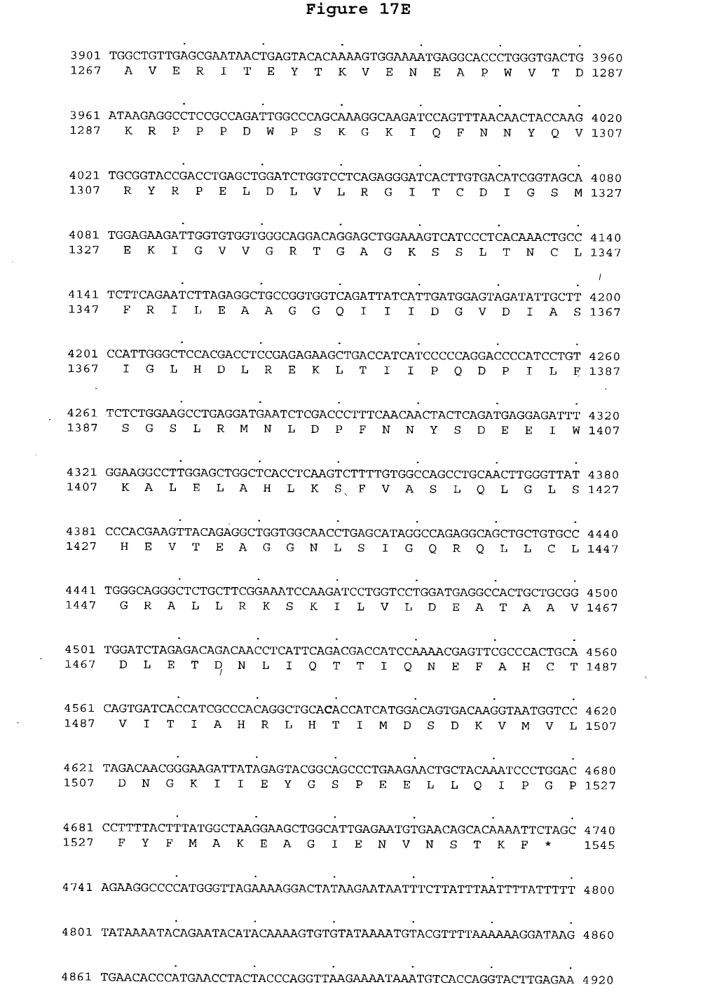

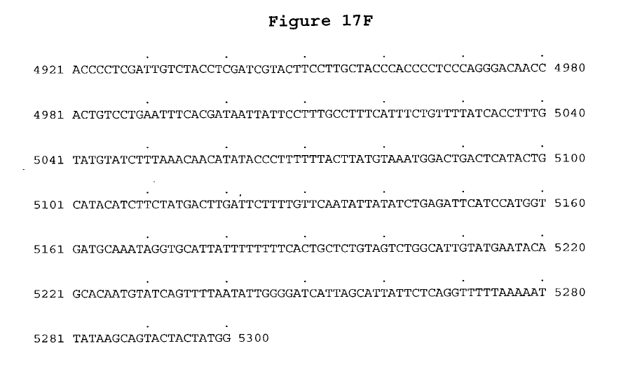

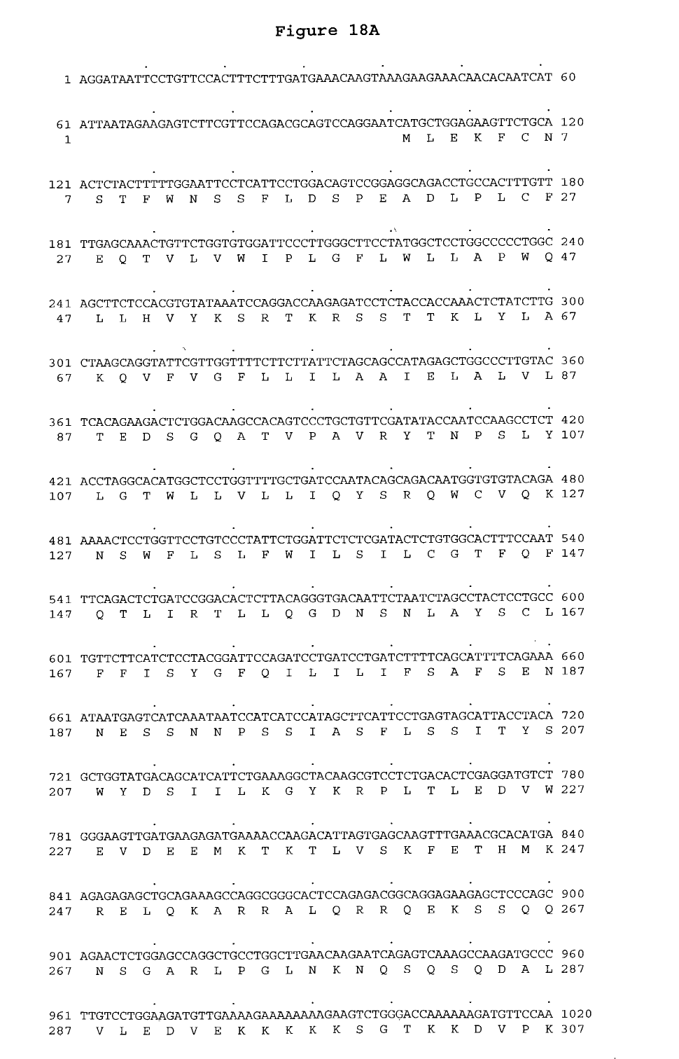

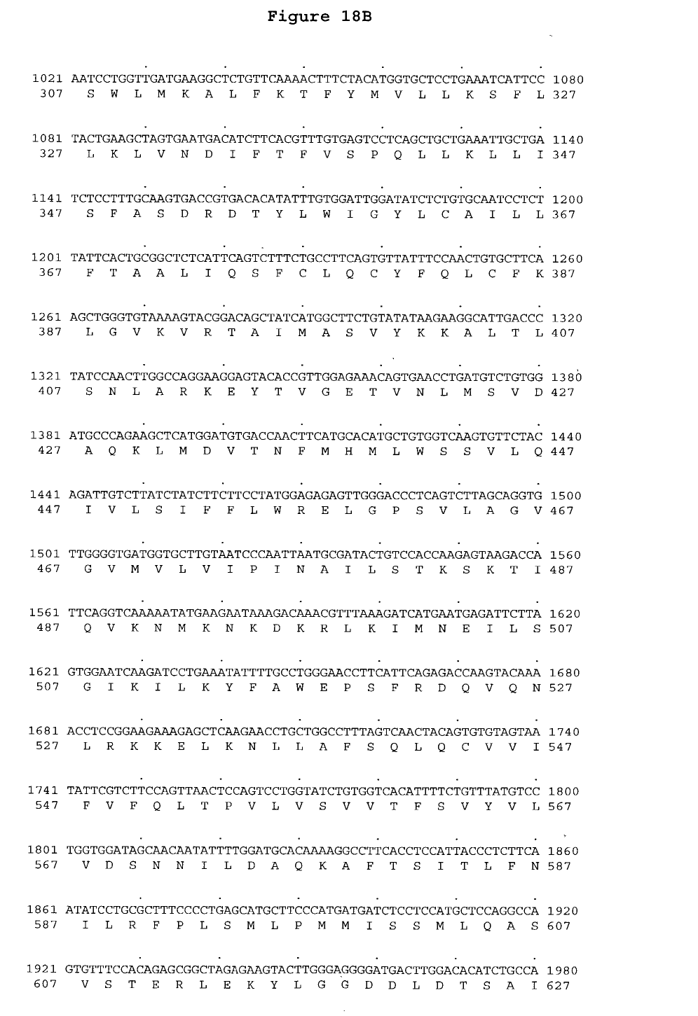

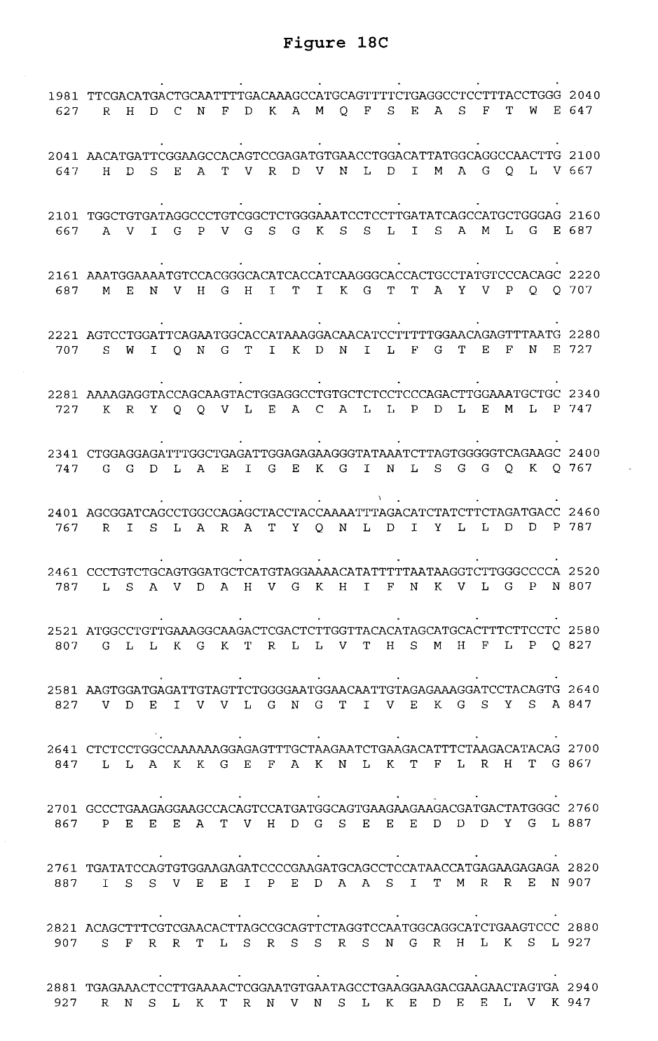

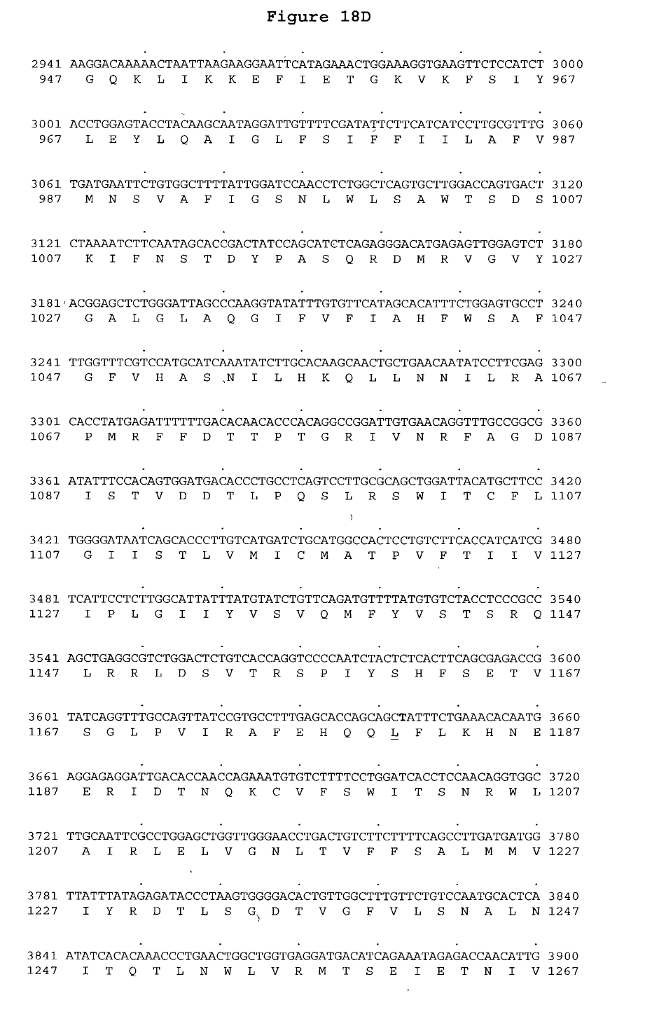

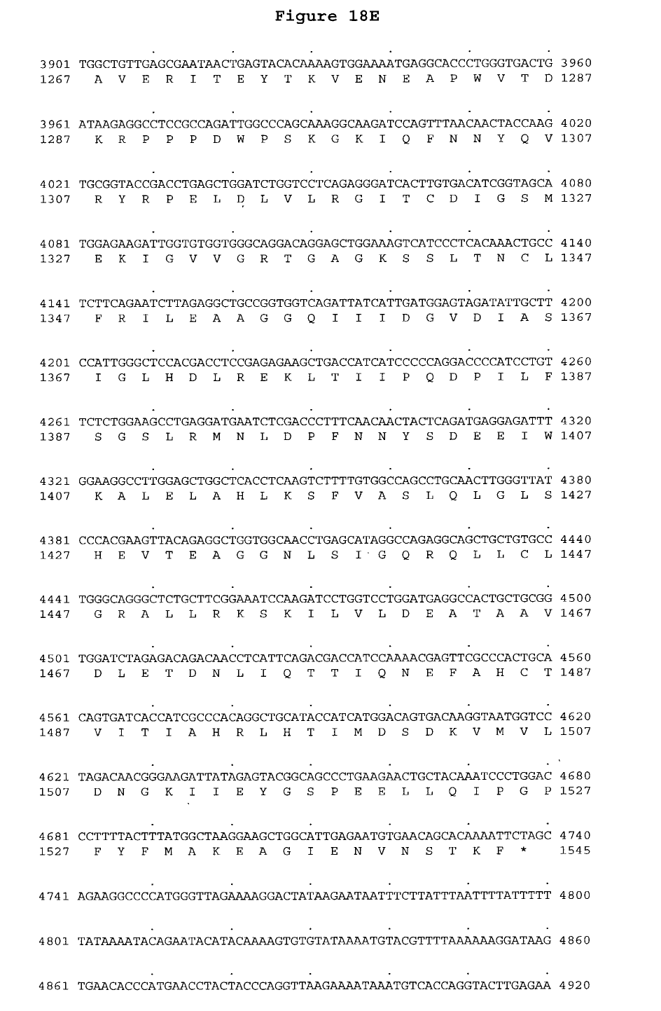

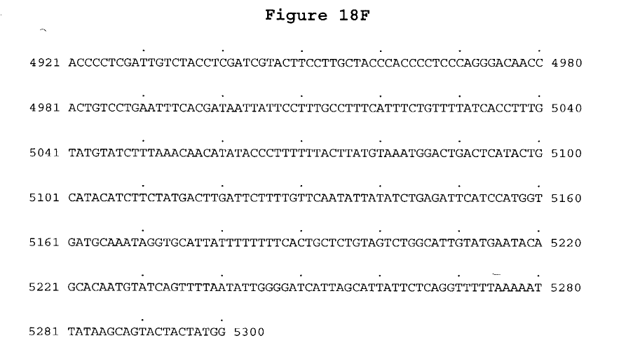

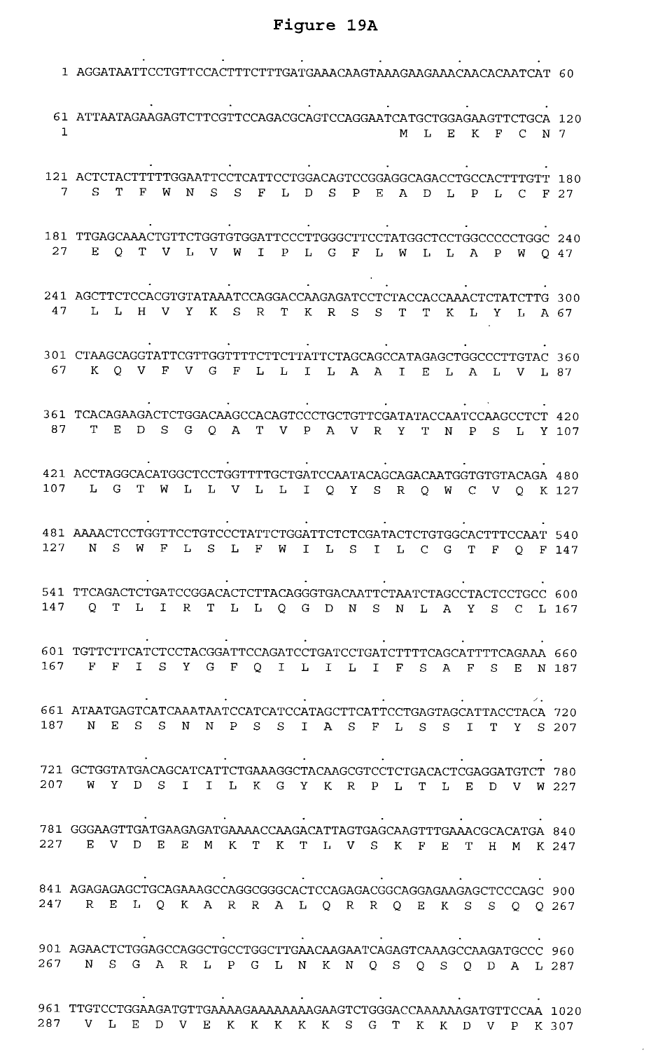

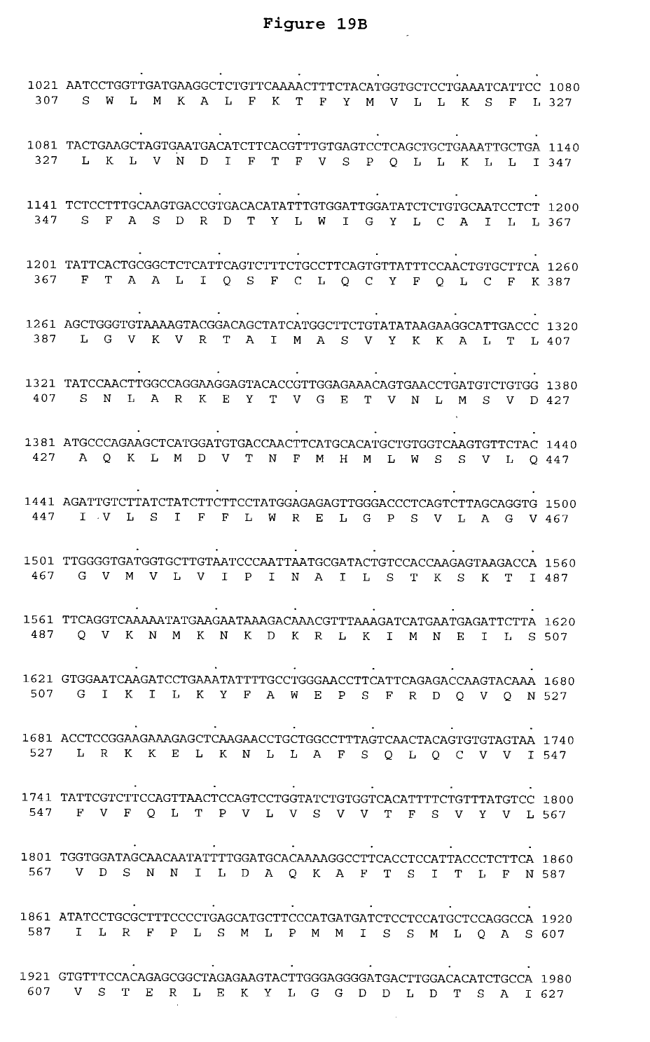

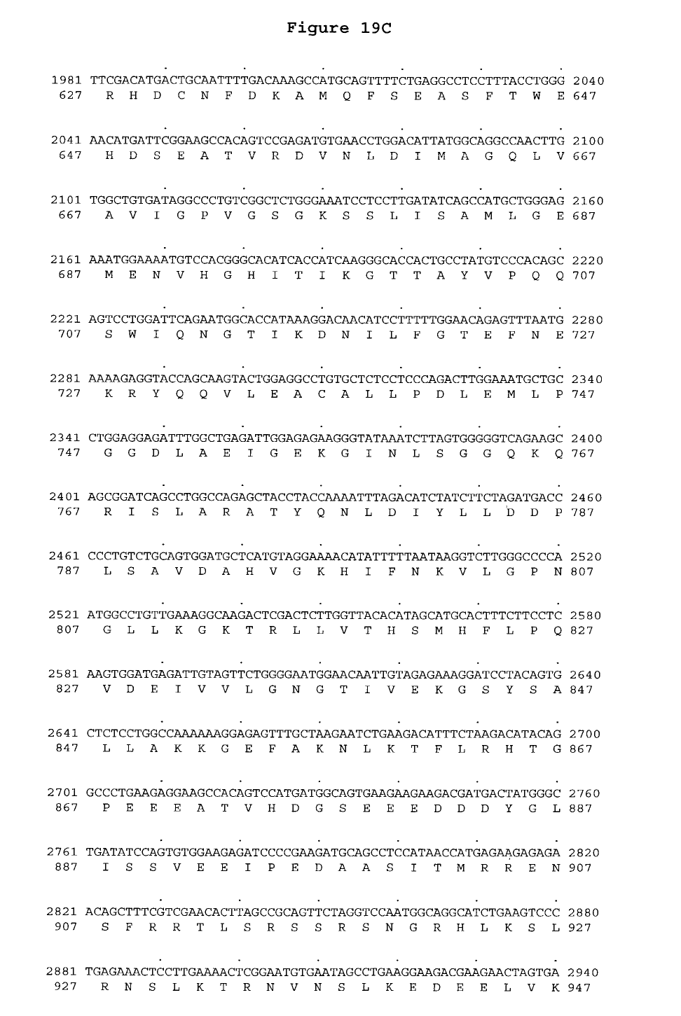

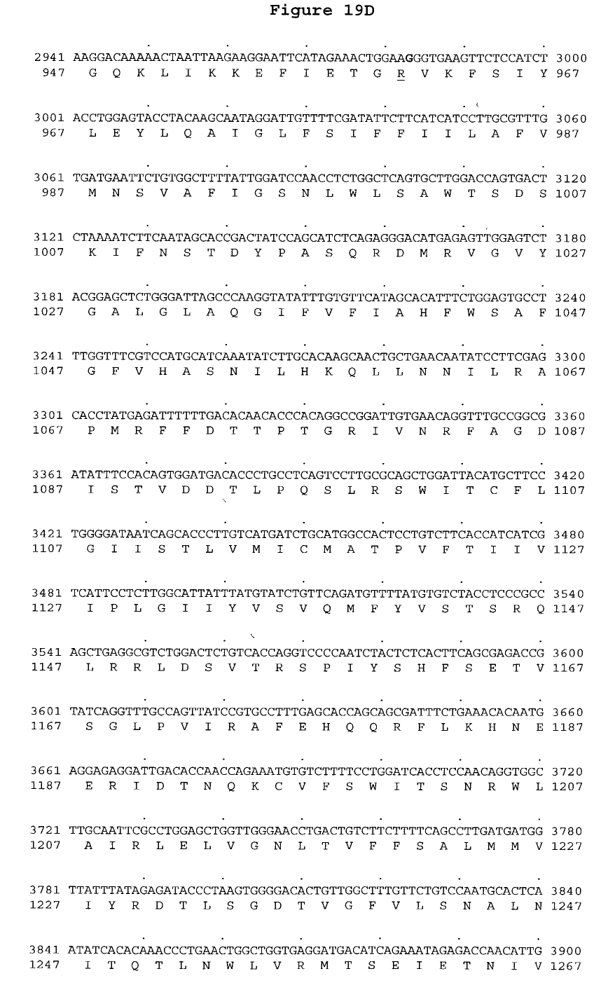

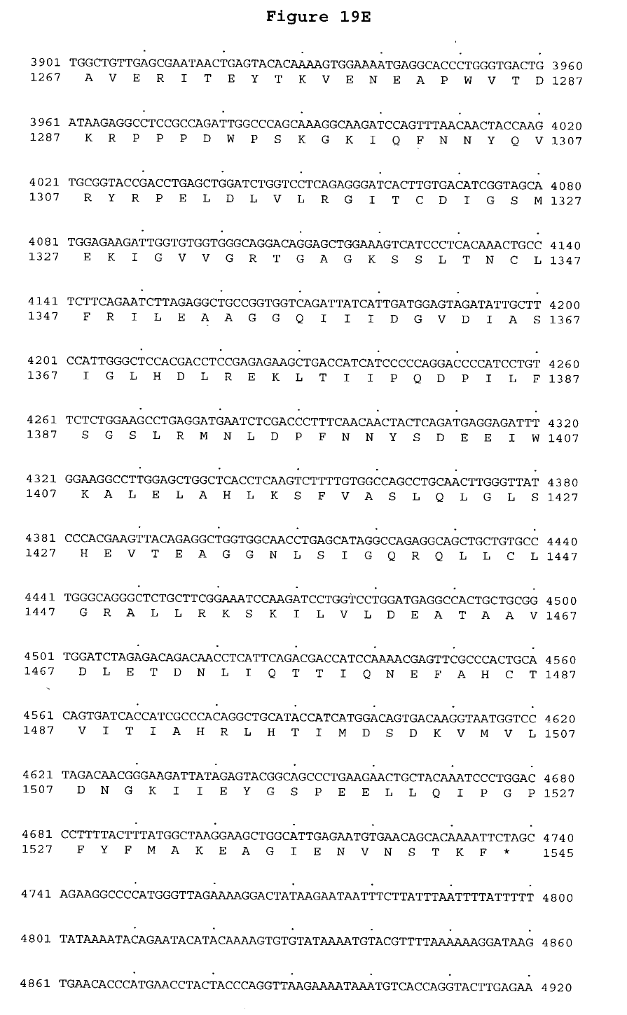

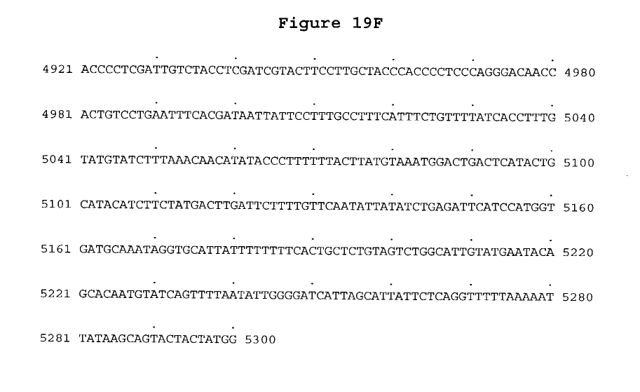

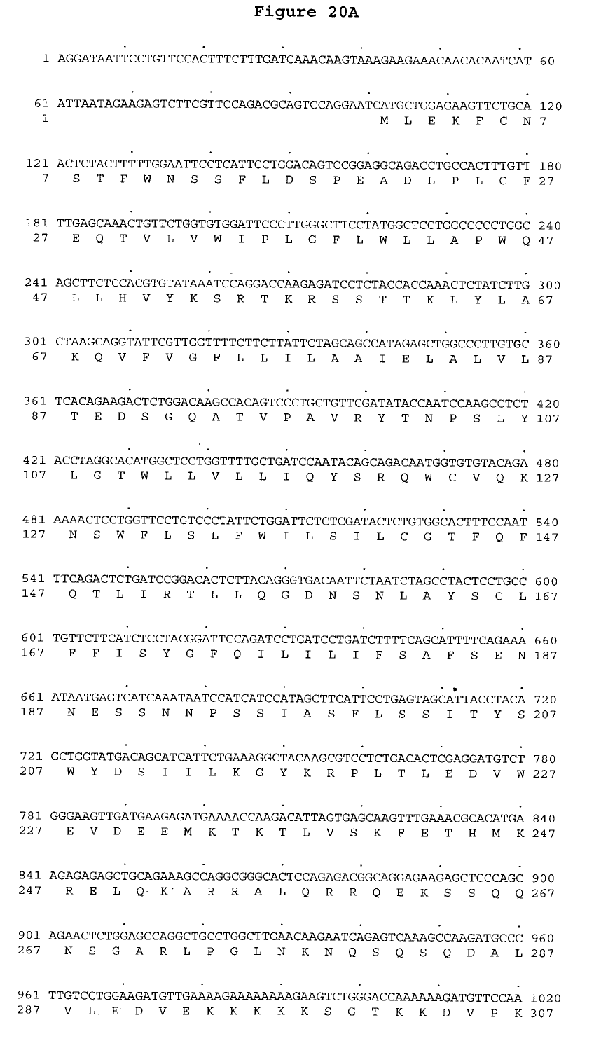

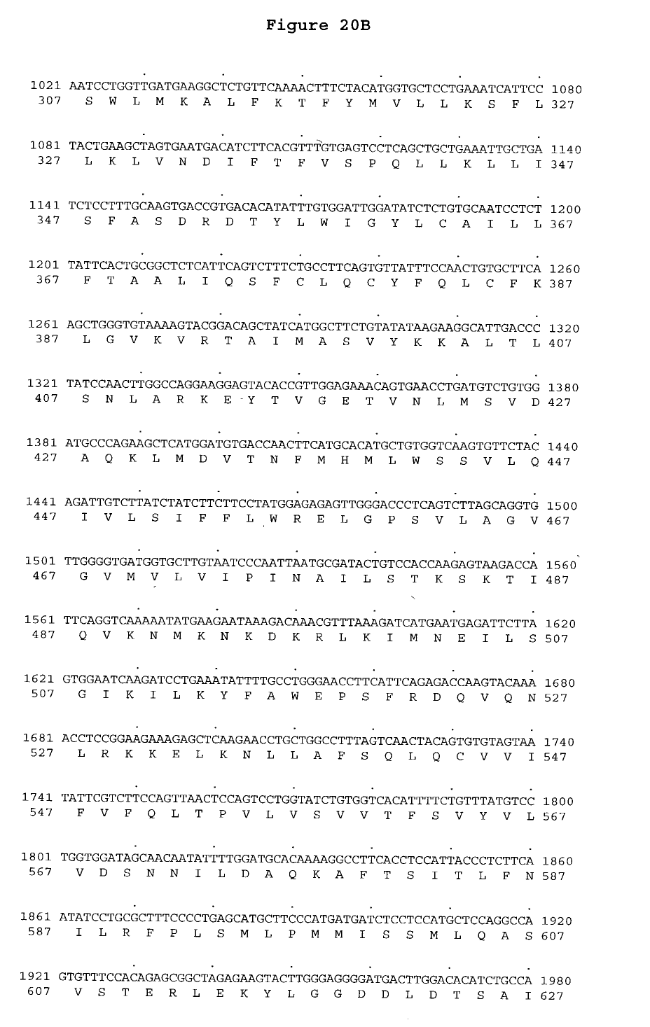

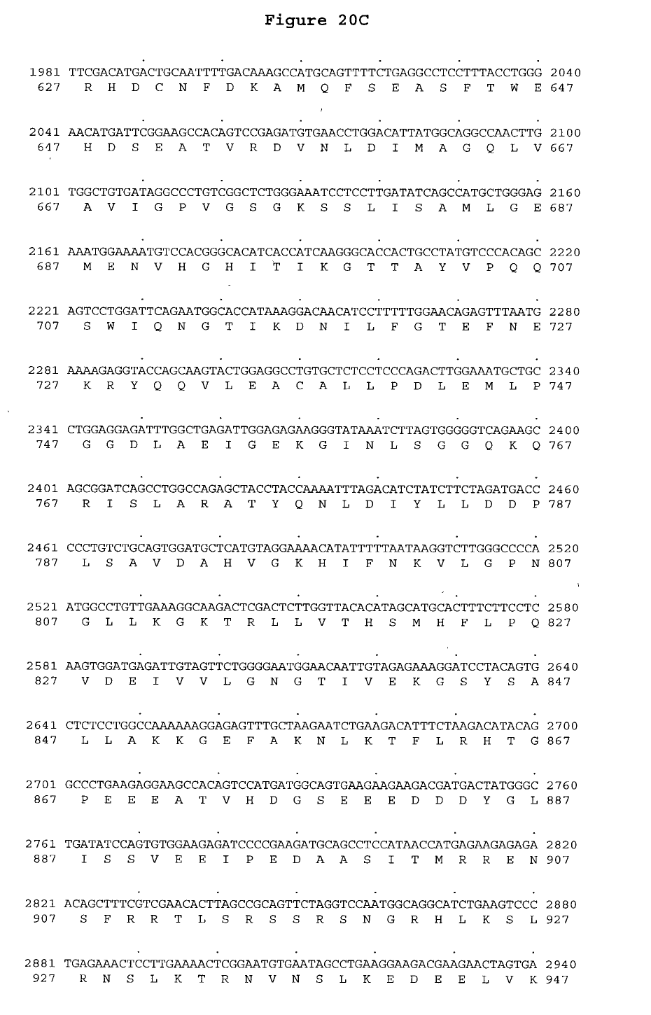

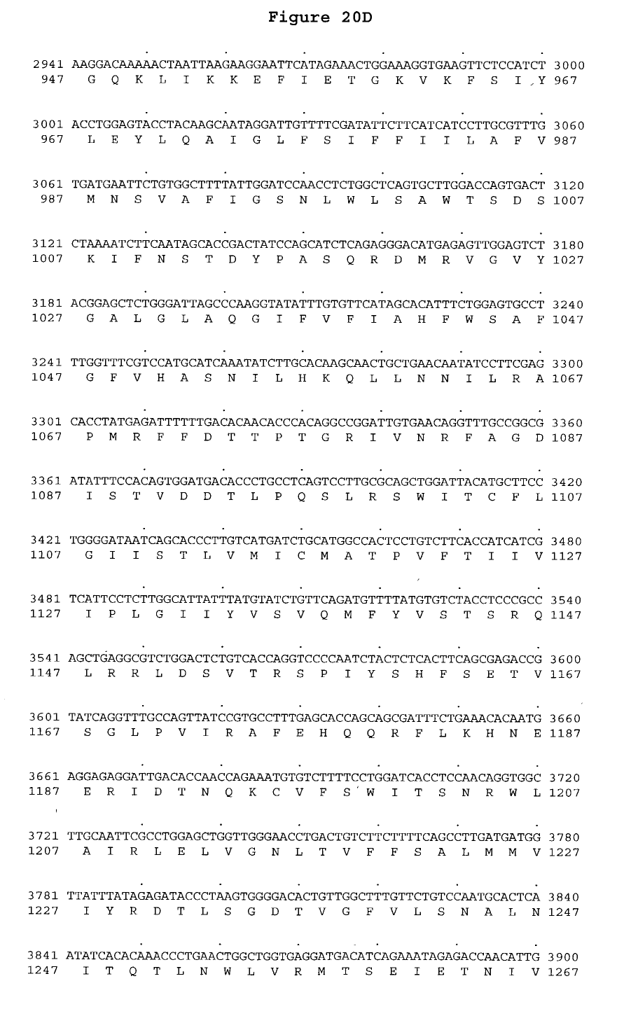

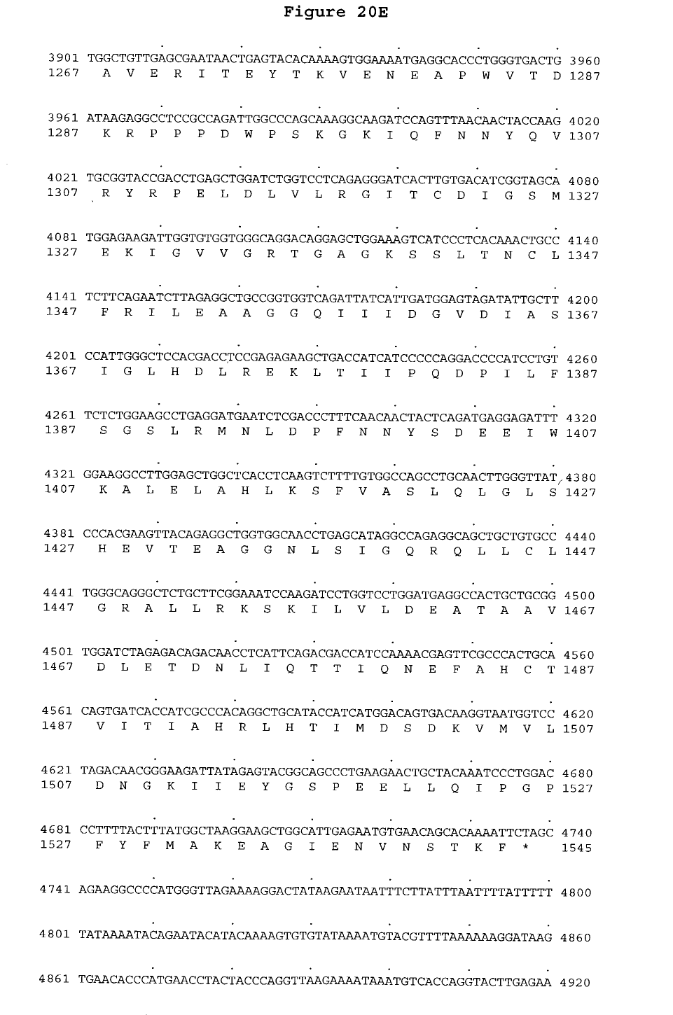

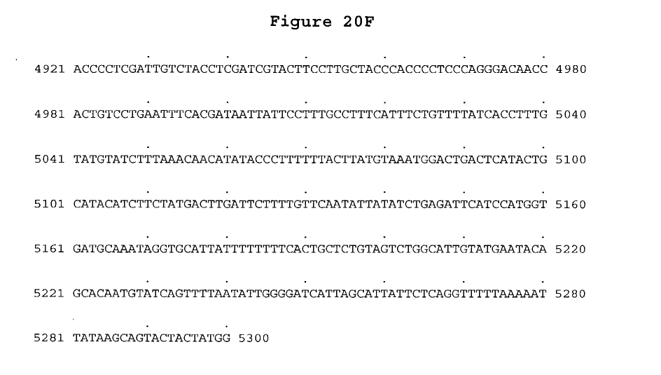

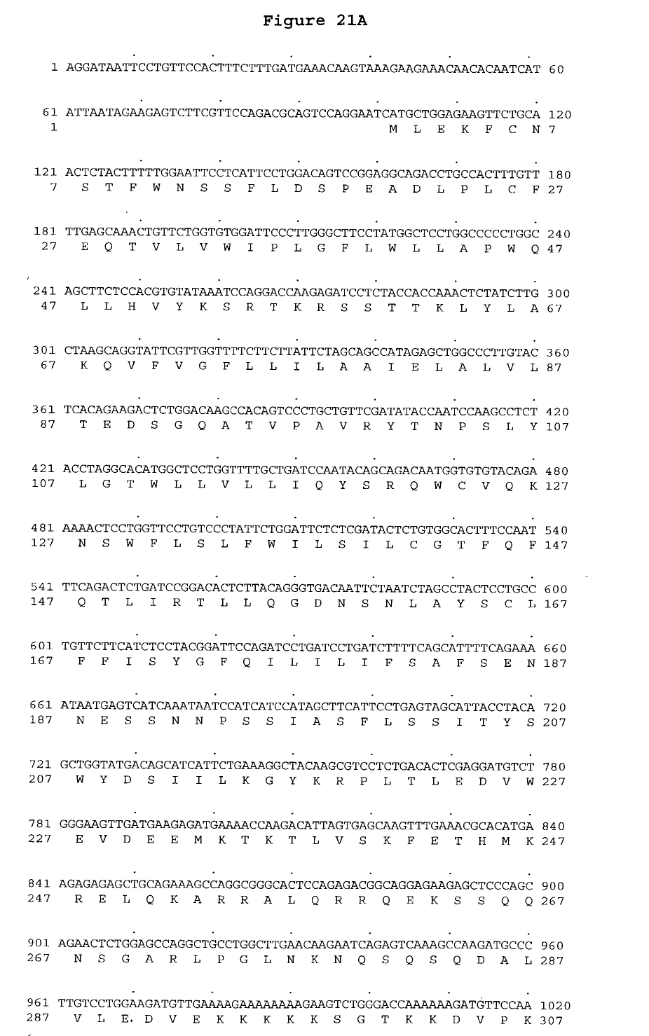

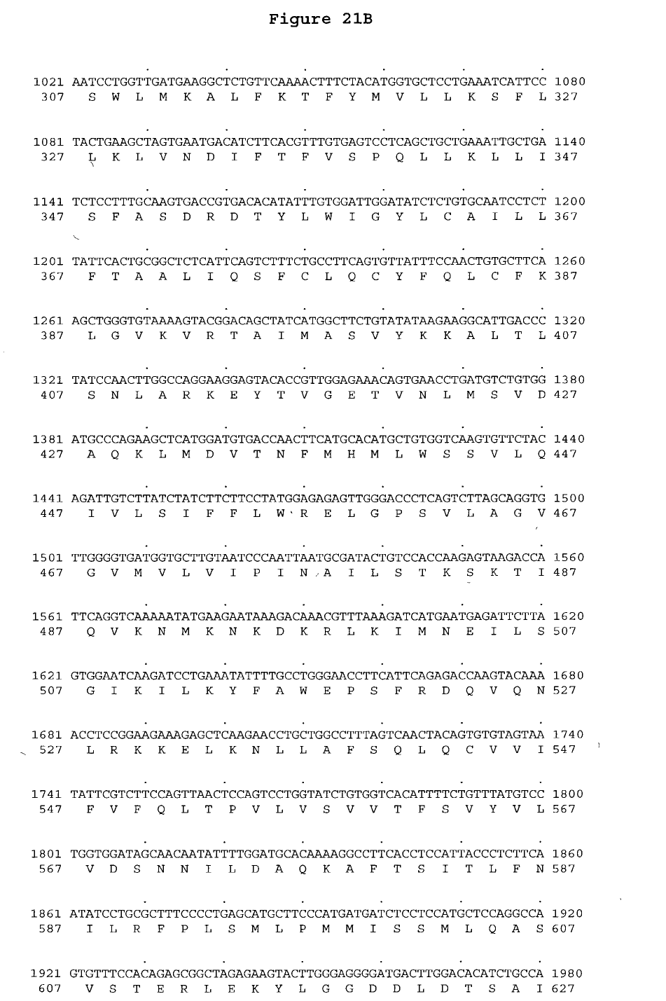

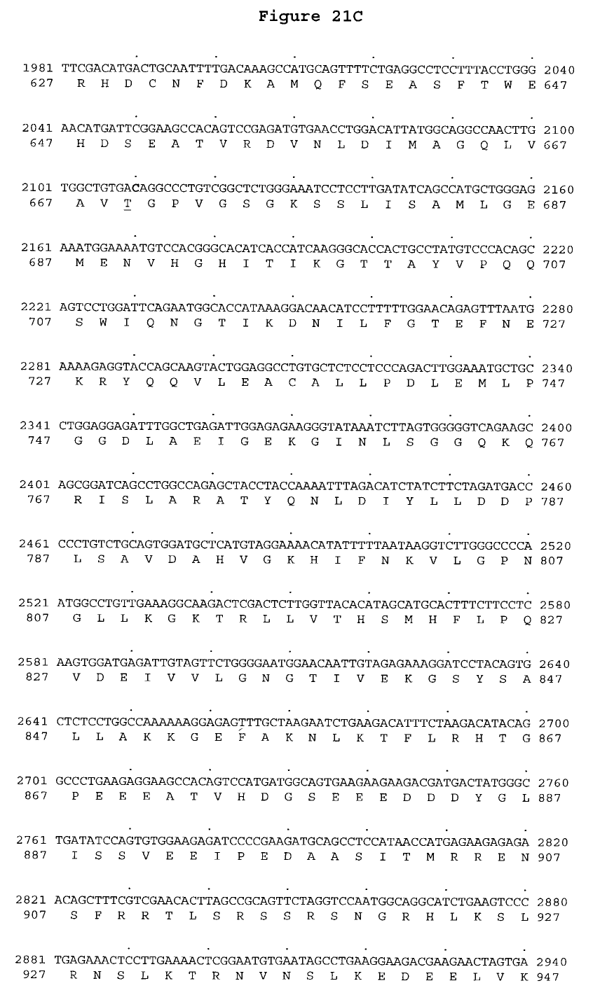

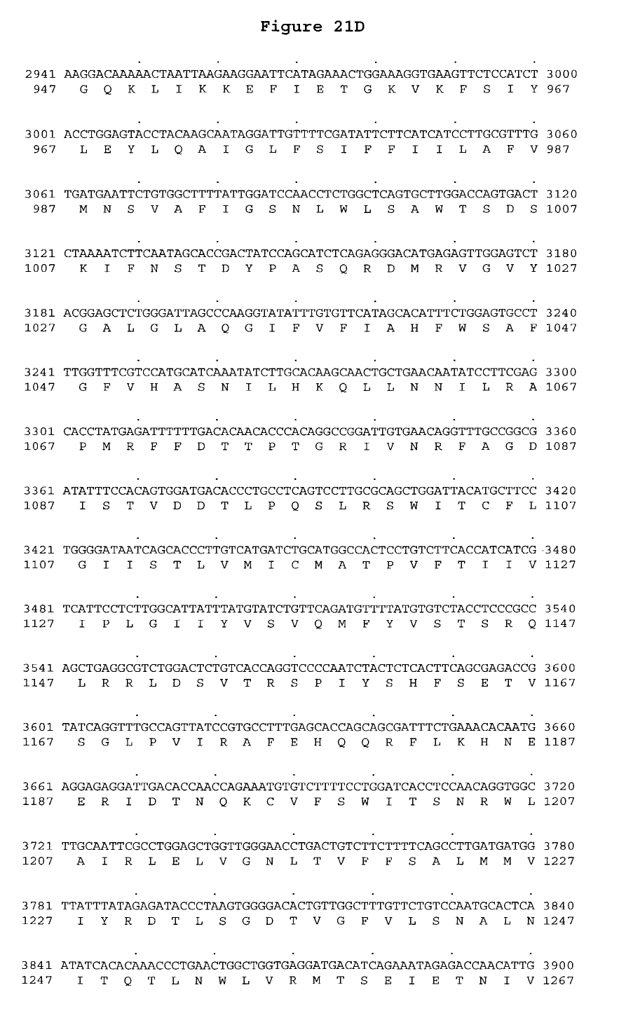

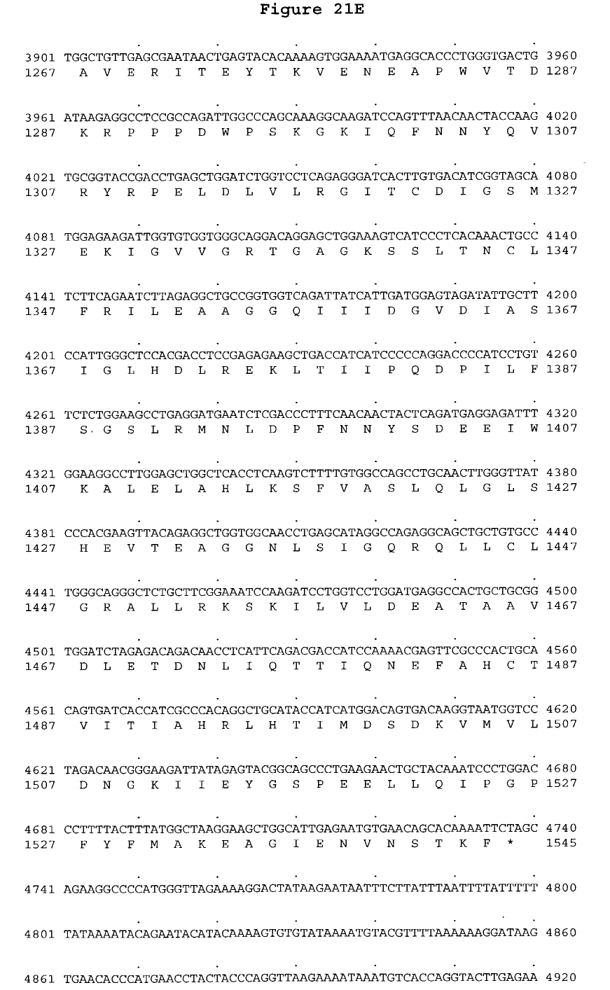

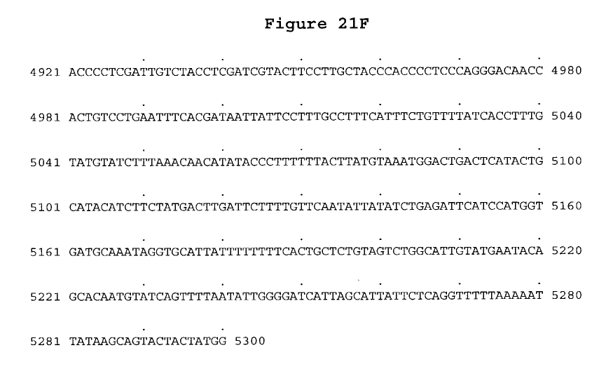

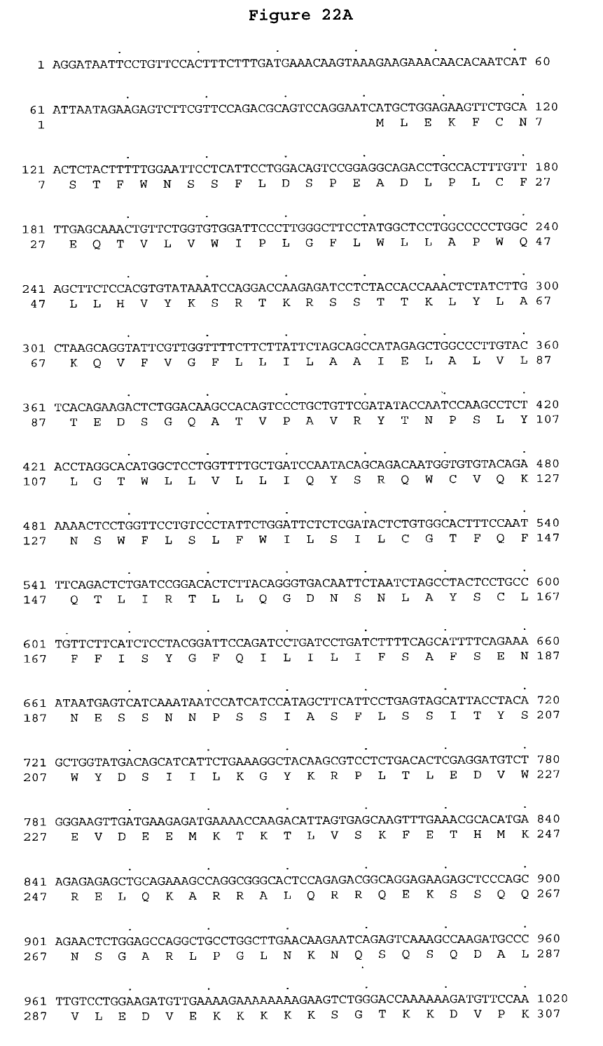

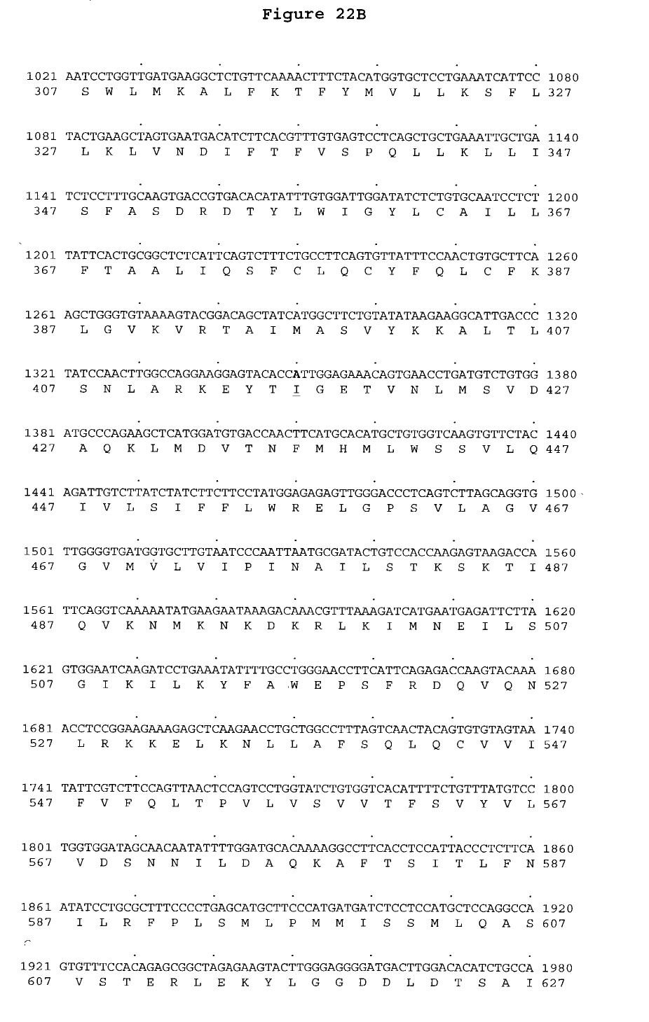

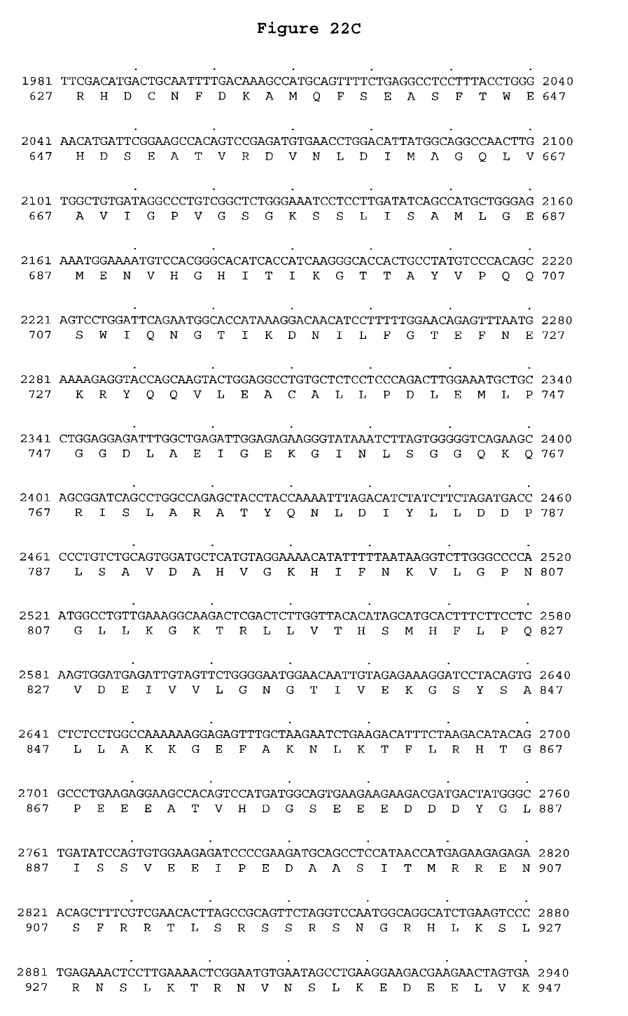

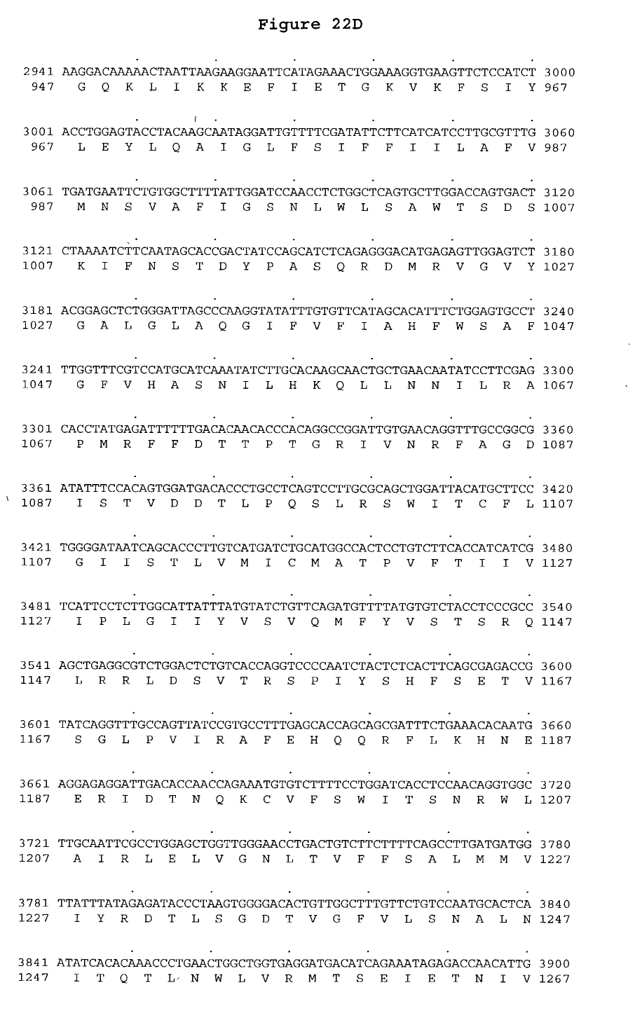

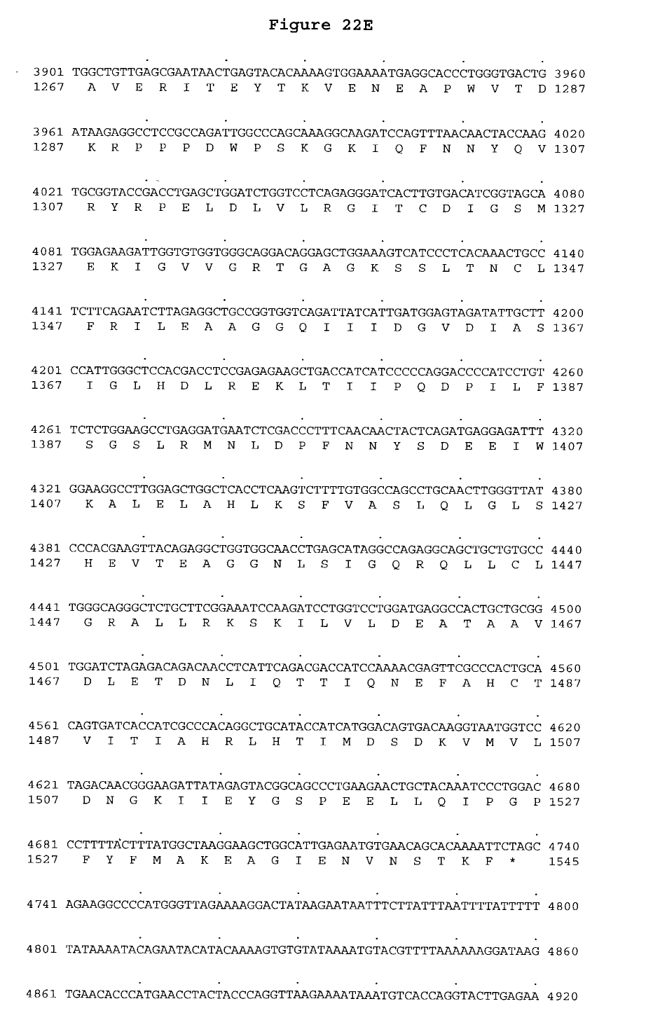

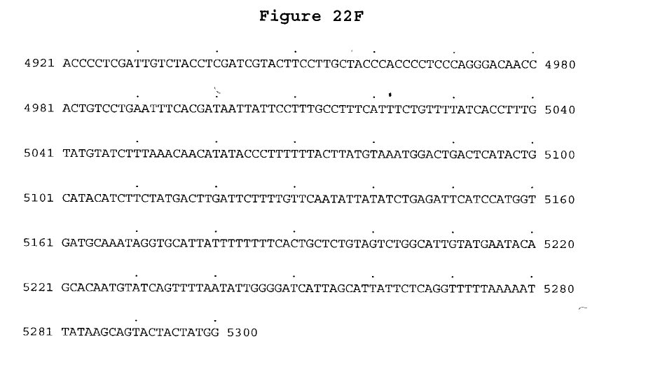

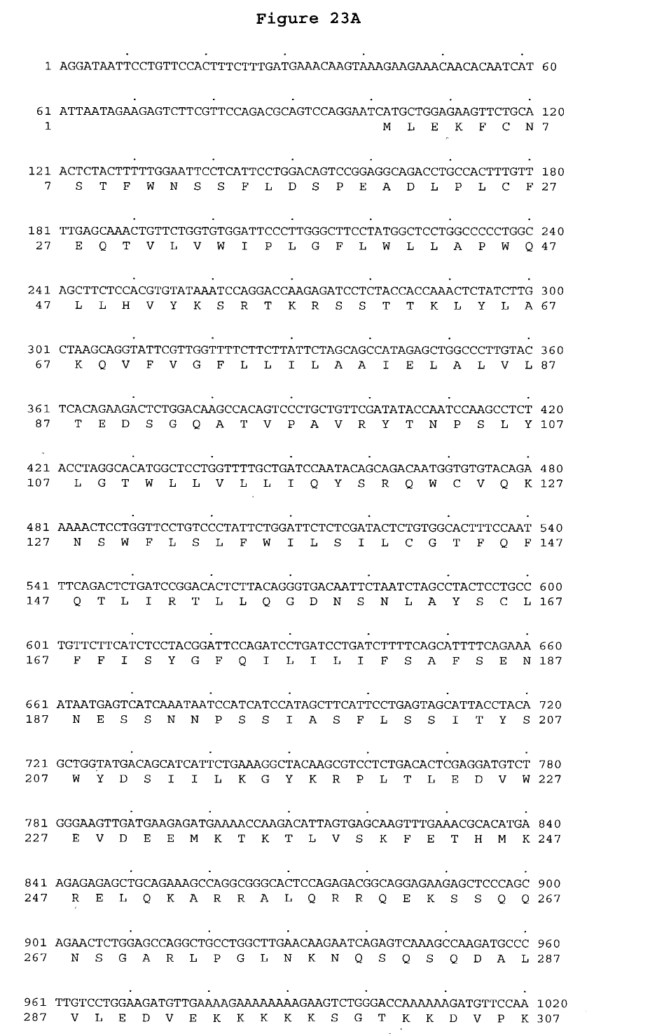

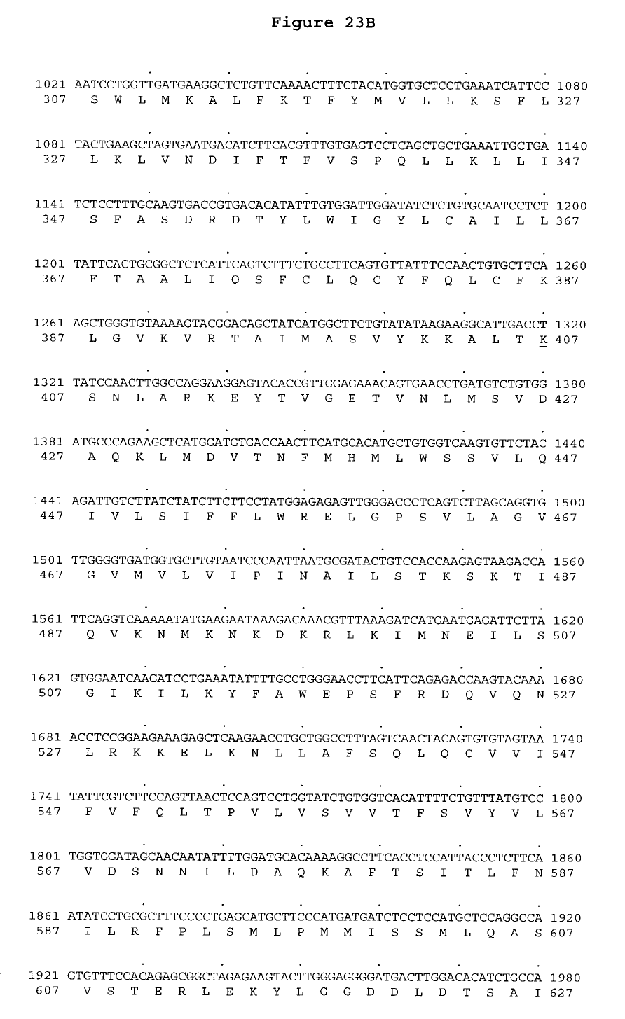

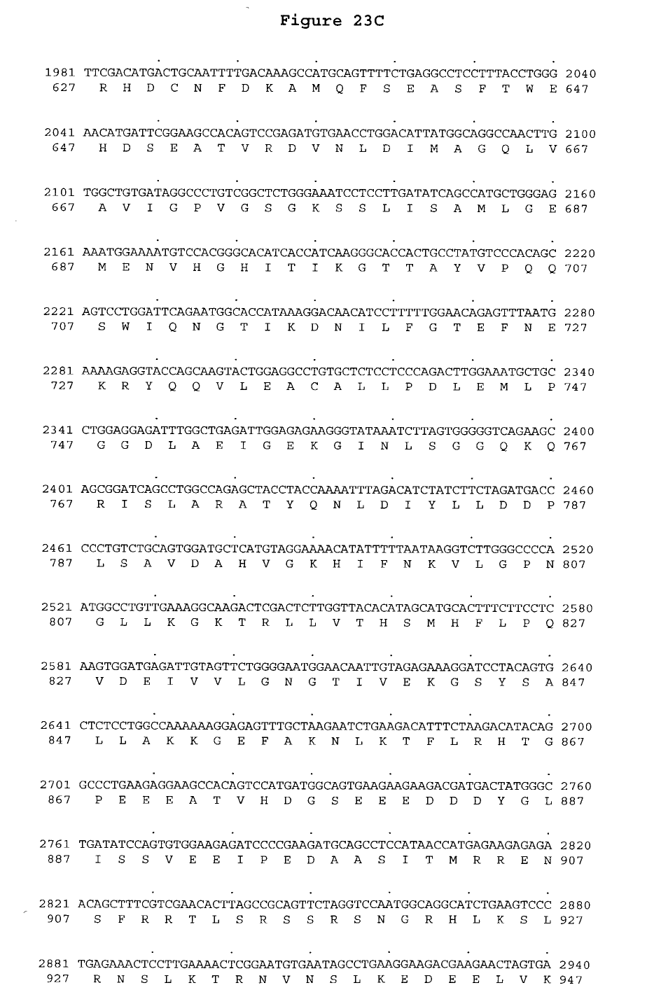

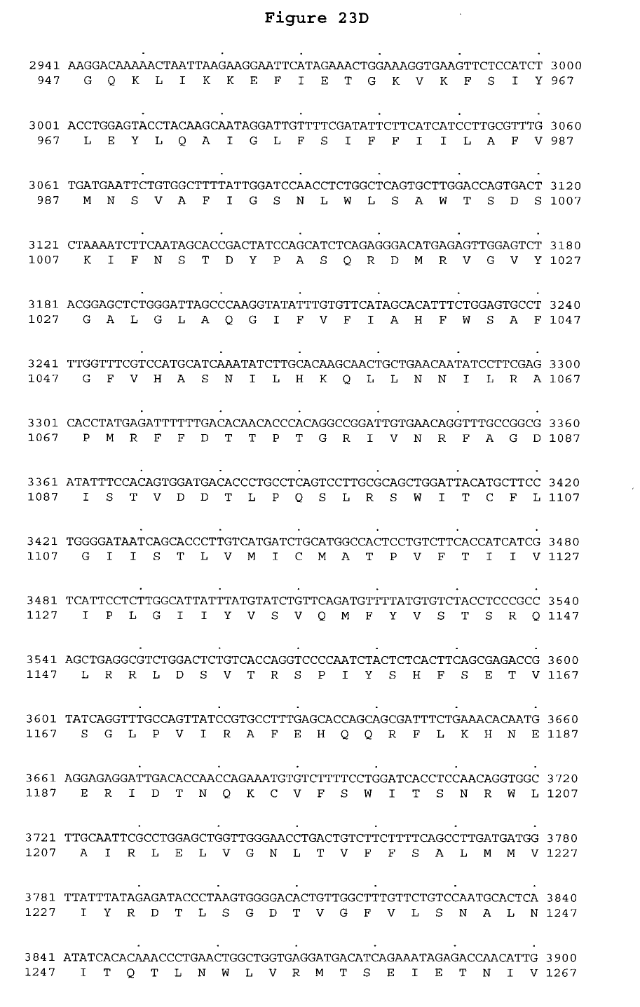

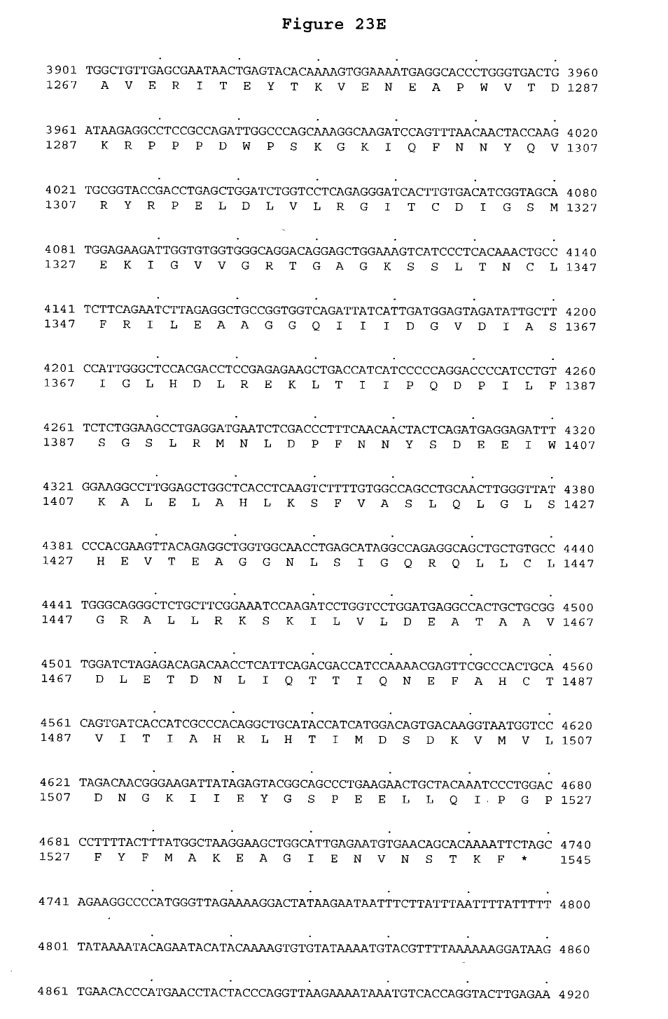

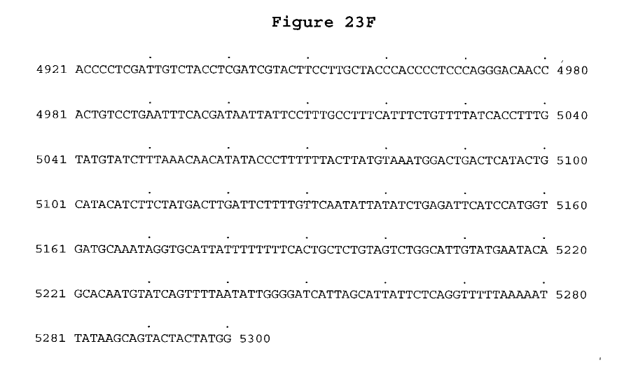

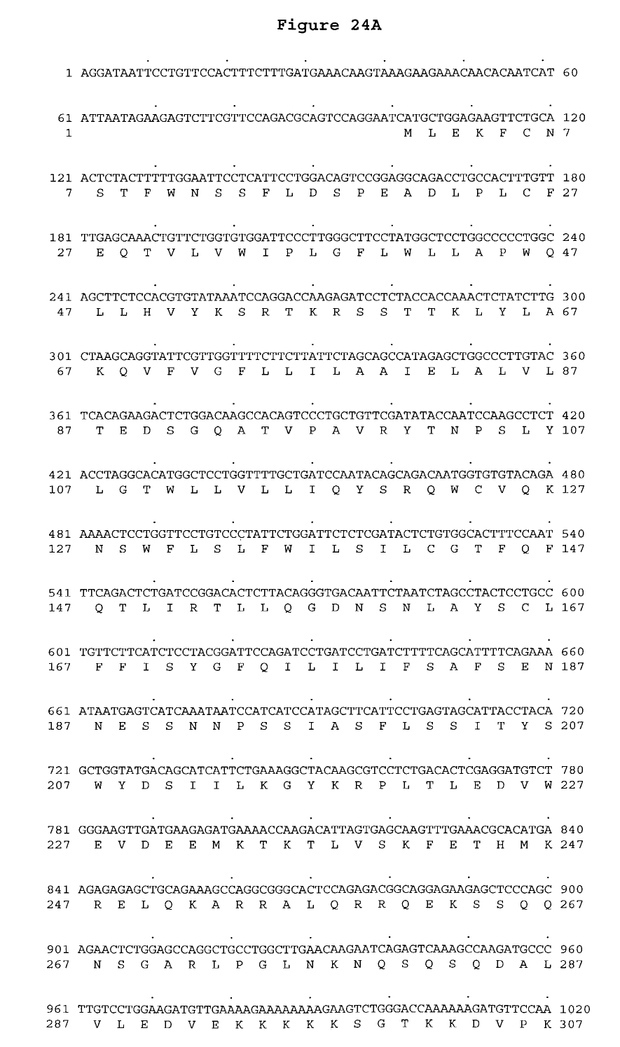

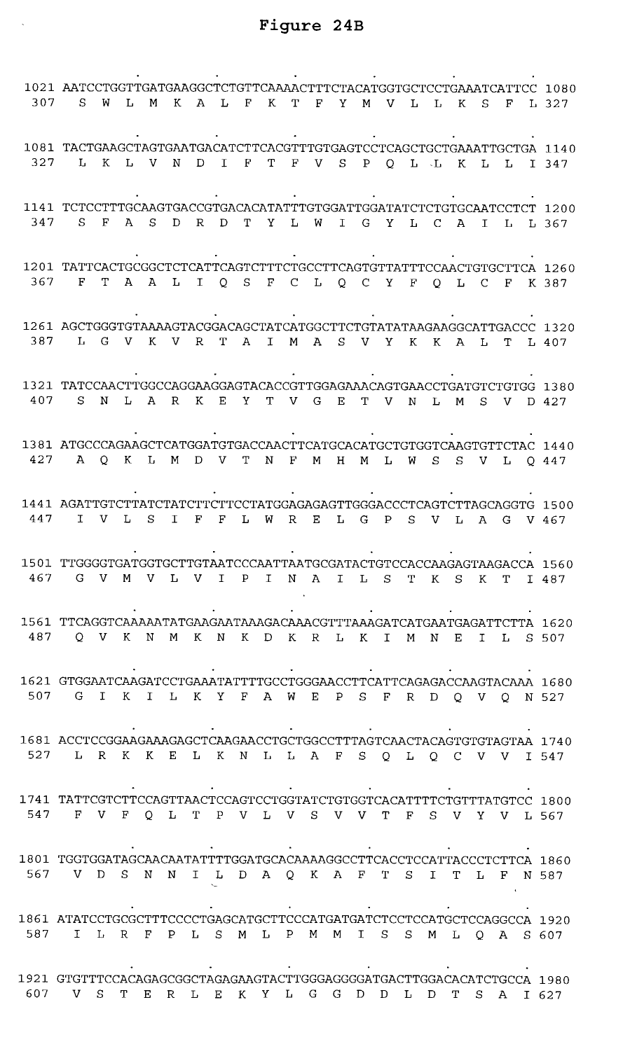

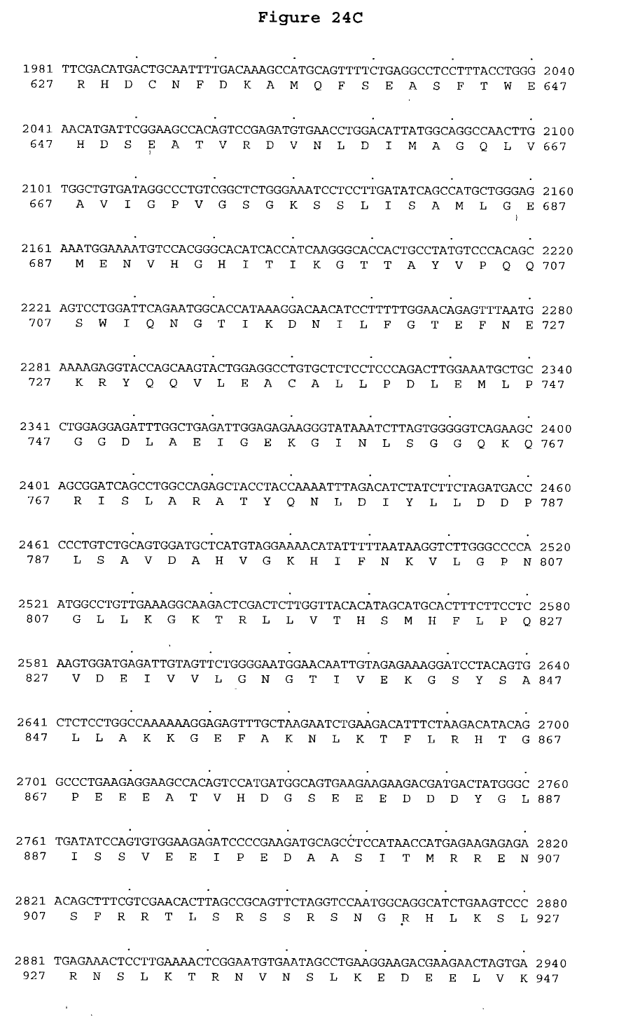

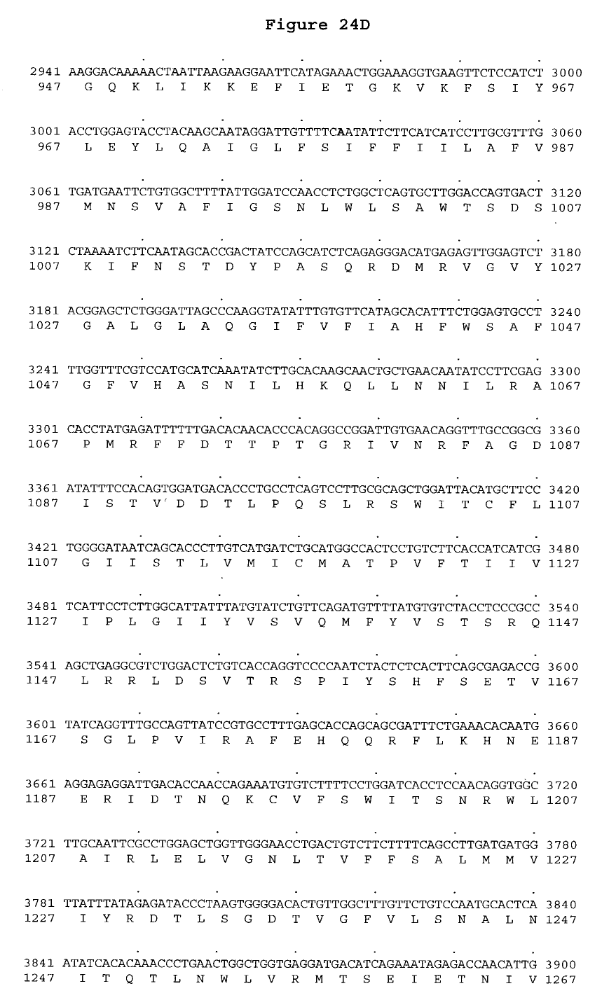

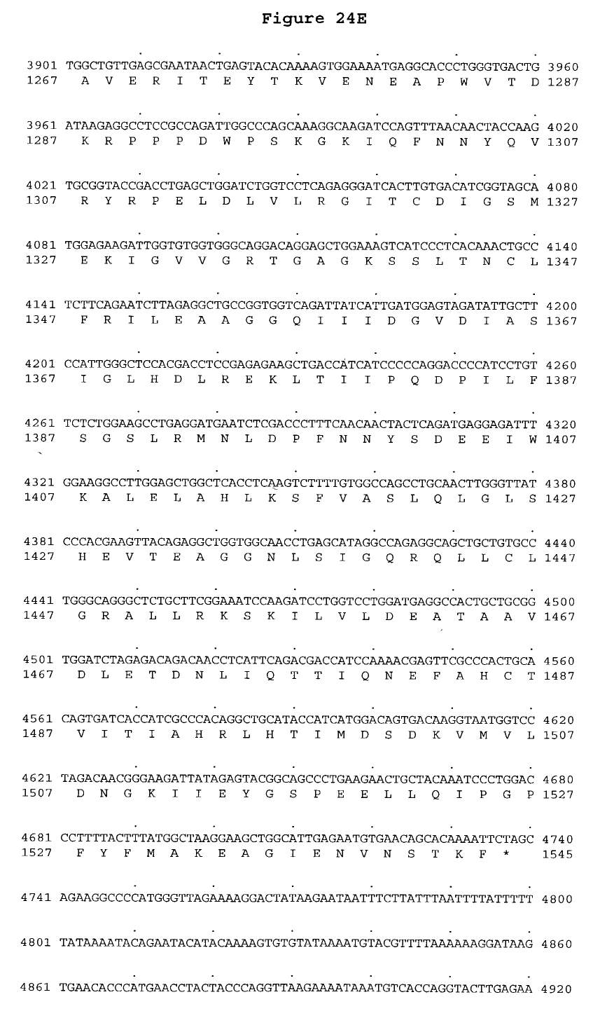

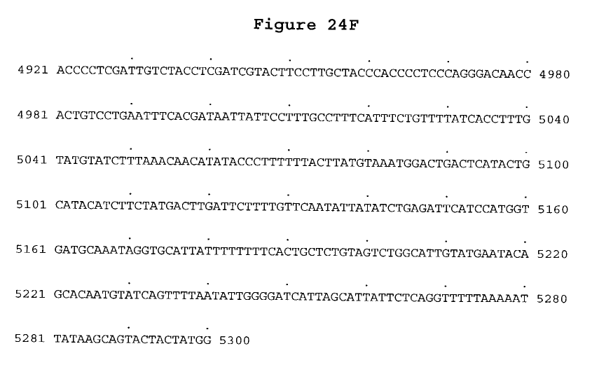

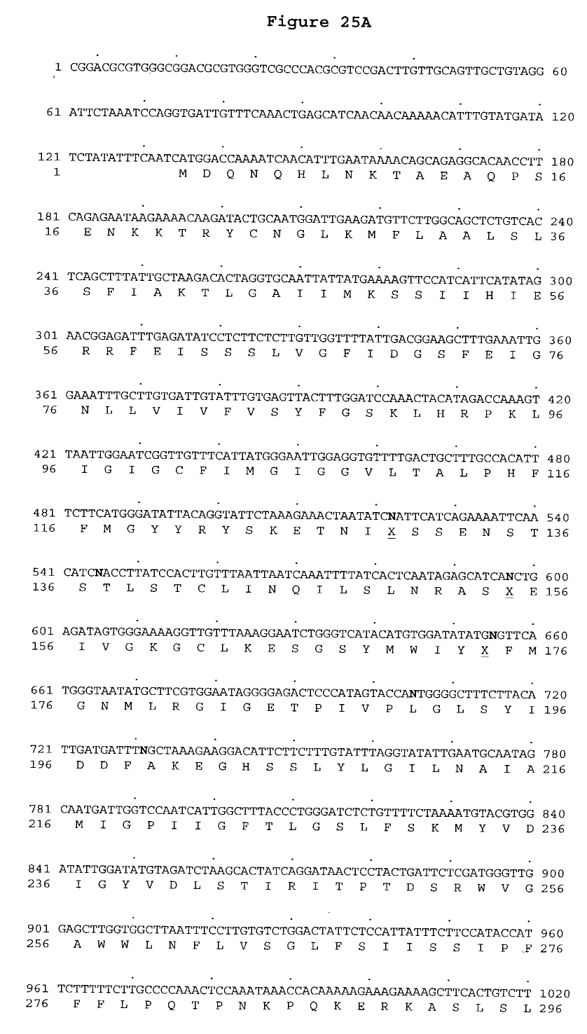

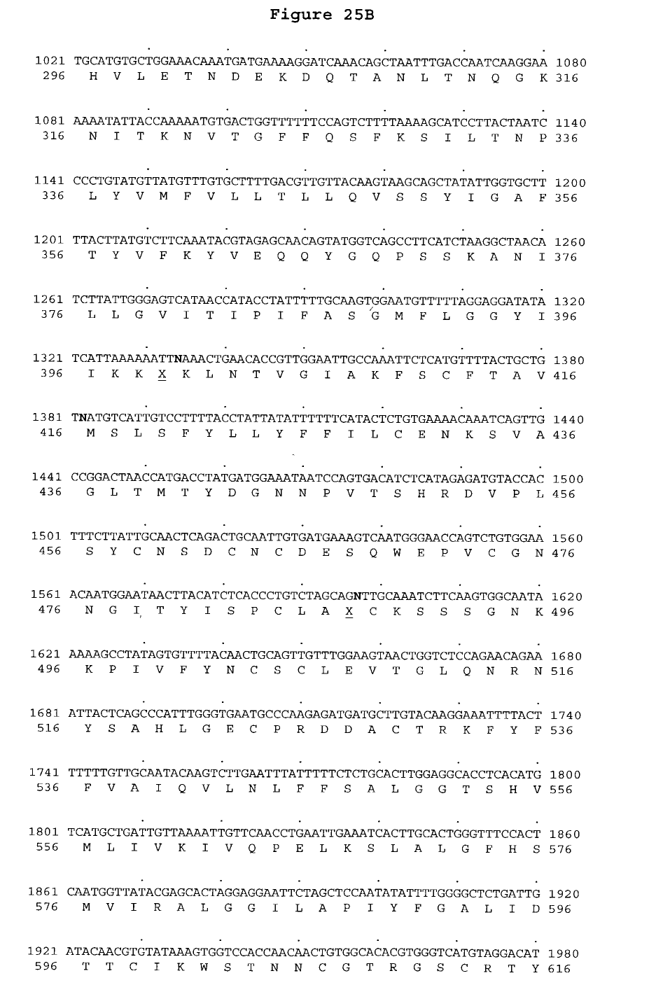

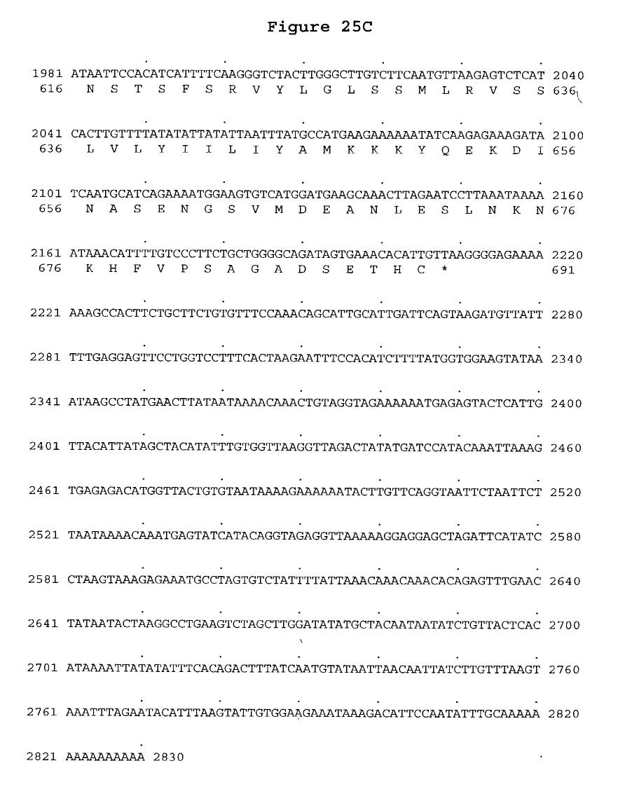

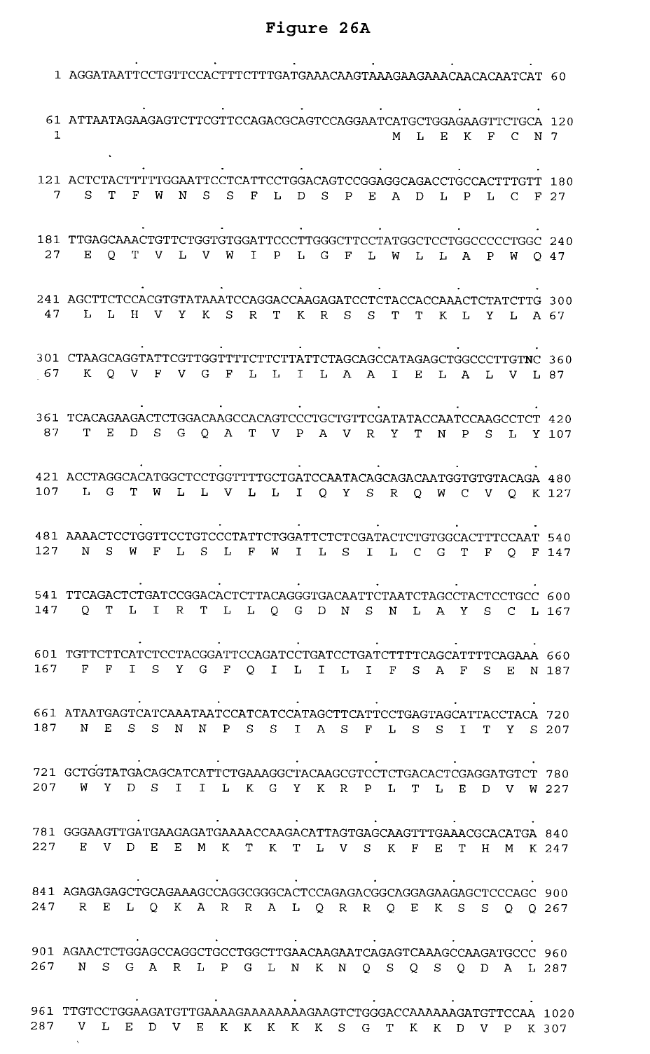

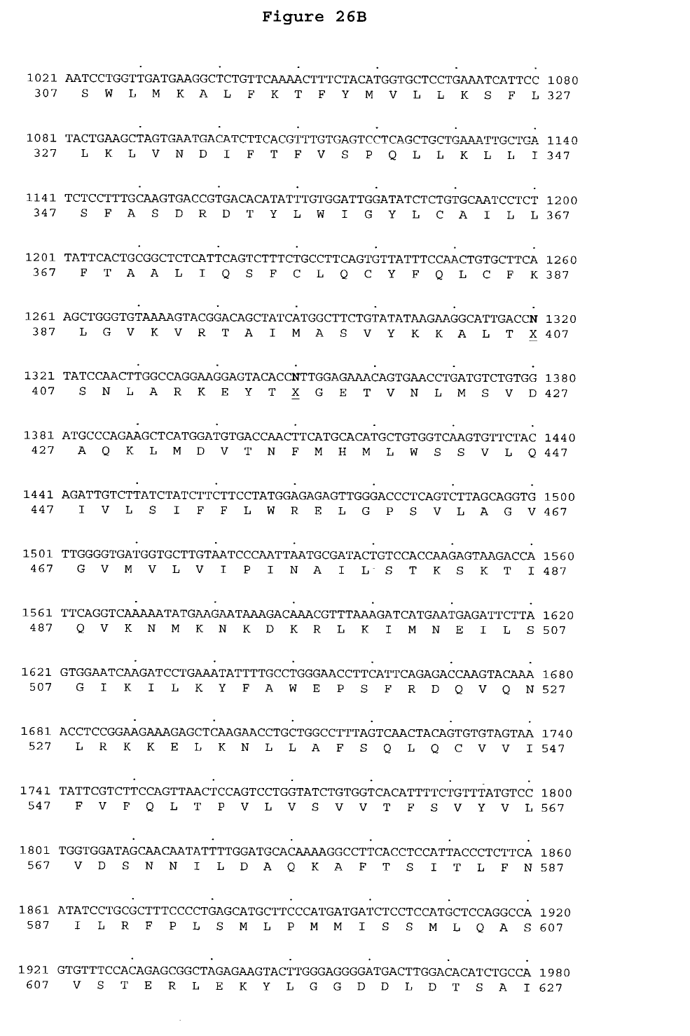

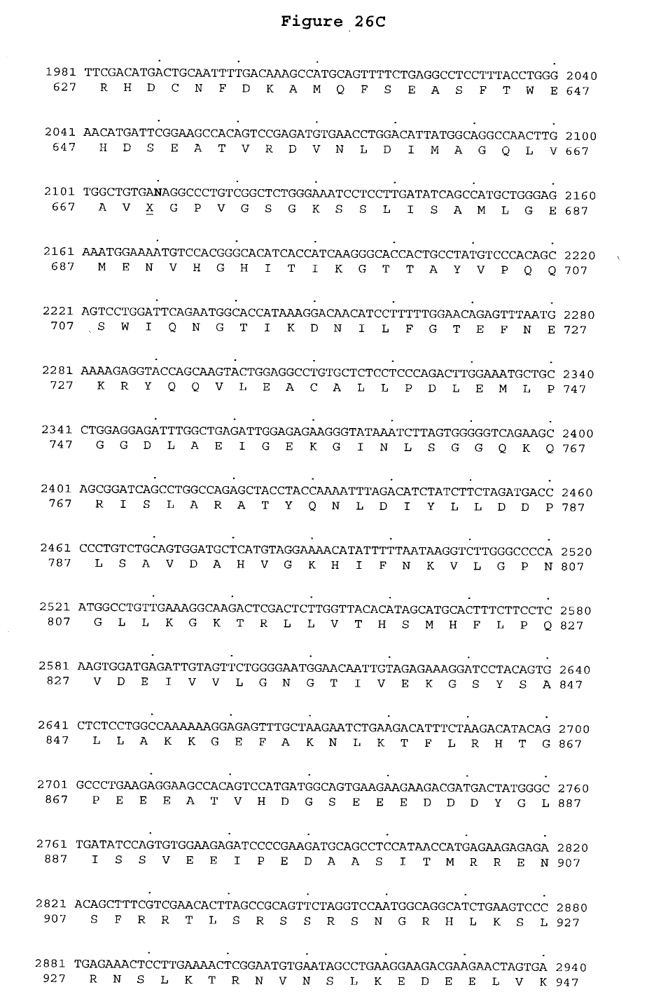

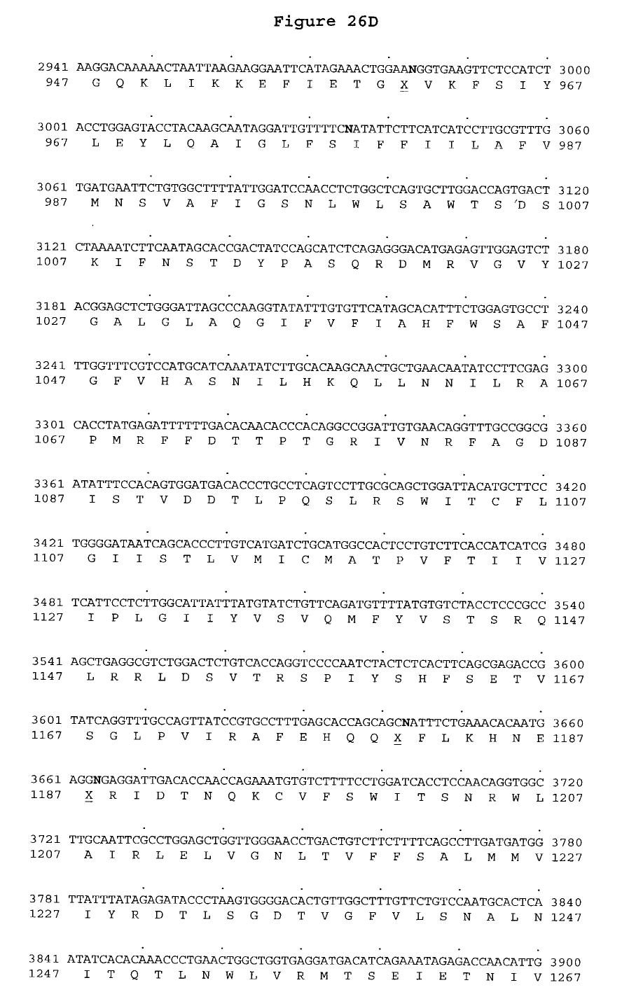

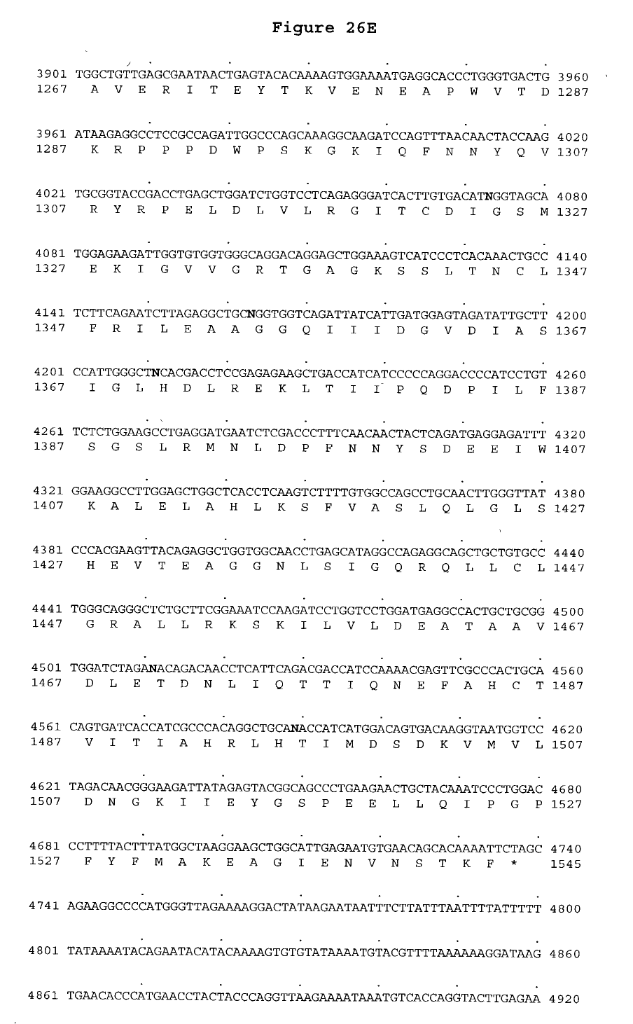

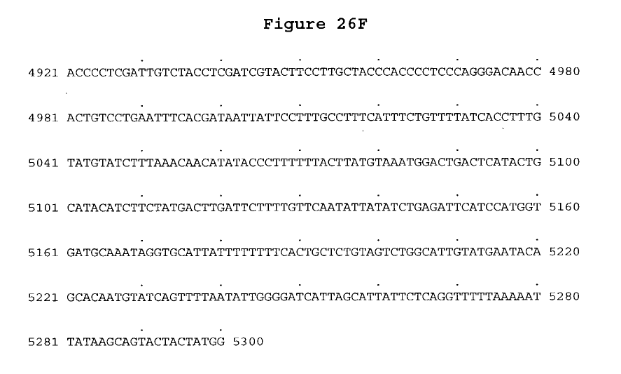

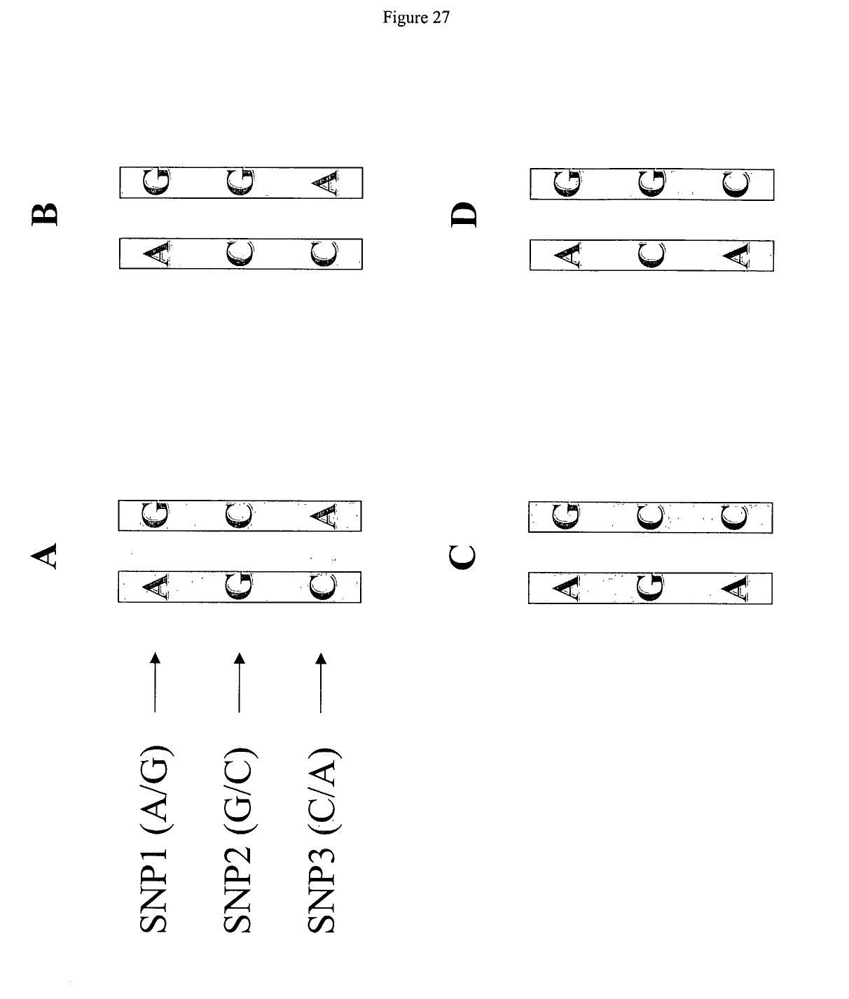

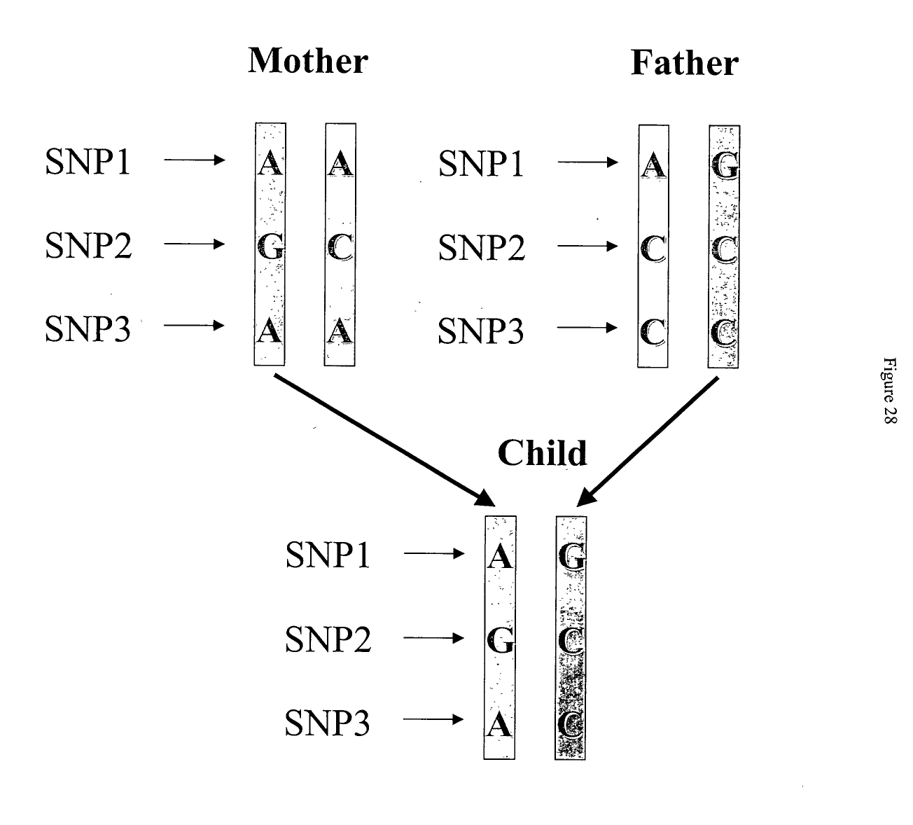

2. Introduction of a mutation by PCR amplification of the cDNA region downstream of the mutation site using a primer including the mutation. (FIG. 8.5.2 in (Cormack 2000)). In the case of introducing the P155T mutation into the human OATP2 protein, the following two primers may be used.

M13 reverse sequencing primer:

(SEQ ID NO:616)

5′-AGCGGATAACAATTTCACACAGGA-3′.
Mutation primer:

(SEQ ID NO:617)

5′-pCAGAAAATTCAACATCAACCTTATCCAC-3′.

Mutation primer contains the mutation (P155T) at the 5′ end and its downstream flanking sequence. M13, reverse sequencing primer hybridizes to the pUC19 vector. Subcloned cDNA comprising the human OATP2 protein is used as a template (described in Step 1). A 100 ul PCR reaction mixture is prepared using 10 ng of the template DNA, 200 uM 4 dNTPs, 1 uM primers, 0.25 U Taq DNA polymerase (PE), and standard Taq DNA polymerase buffer. Typical PCR cycling condition are as follows:

| | |
|---|---|
| 20-25 cycles: | 45 sec, 93 degrees |
| | 2 min, 50 degrees |
| | 2 min, 72 degrees |
| 1 cycle: | 10 min, 72 degrees |

After the final extension step of PCR, 5 U Klenow Fragment is added and incubated for 15 min at 30 degrees. The PCR product is then digested with the restriction enzyme, EcoRI.

3. PCR amplification of the upstream region is then performed, using subcloned cDNA as a template (the product of Step 1). This PCR is done using the following two primers:

M13 forward sequencing primer:

(SEQ ID NO:618)

5′-CGCCAGGGTTTTCCCAGTCACGAC-3′.
Flanking primer:

(SEQ ID NO:619)

5′-pGTCTTTTAAGTTGTAGTTGGAATAGGTG-3′.

Flanking primer is complimentary to the upstream flanking sequence of the P155T mutation. M13 forward sequencing primer hybridizes to the pUC19 vector. PCR conditions and Klenow treatments follow the same procedures as provided in Step 2, above. The PCR product is then digested with the restriction enzyme, HindIII.

4. Prepare the pUC19 vector for cloning the cDNA comprising the polymorphic site. Digest pUC19 plasmid DNA with EcoRI and HindII. The resulting digested vector fragment may then be purified using techniques well known in the art, such as gel purification, for example.

5. Combine the products from Step 2 (PCR product containing mutation), Step 3 (PCR product containing the upstream region), and Step 4 (digested vector), and ligate them together using standard blunt-end ligation conditions (Sambrook, Fritsch et al. 1989).

6. Transform the resulting recombinant plasmid from Step 5 into E.coli competent cells using methods known in the art, such as, for example, the transformation methods described in Sambrook, Fritsch et al. 1989.

7. Analyze the amplified fragment portion of the plasmid DNA by DNA sequencing to confirm the point mutation, and absence of any other mutations introduced during PCR. The method of sequencing the insert DNA, including the primers utilized, are described herein or are otherwise known in the art.

The skilled artisan would appreciate that the above method may be applied to engineering the other novel polymorphic bradykinin associated genes of the present invention through the simple subsitution of applicable mutation, flanking, PCR, and sequencing primers for each specific gene and/or polymorphism. Some of these primers may be selected from any one of the applicable primers provided in Tables VIII and/or IX, may be designed using the Primer3 program (Rozen S 2000), or designed manually, as described. Such primers may preferably comprise at least a portion of any one of the polynucleotide sequences of the present invention.

Moreover, the skilled artisan would appreciate that the above method may be applied to engineering more than one polymorphic nucleic acid sequence of the present invention into the novel polymorphic organic anion transport genes of the present invention. For example, the OATP2 cDNA could be engineered to comprise the D130Y encoding polymorphism (SNP_ID:AE103s1), or the T129C encoding polymorphism (SNP_ID: PS100s9), or engineered to comprise both the D130Y and P155T polymorphisms. Such an engineered gene could be created through succesive rounds of site-directed mutagenesis, as described in Steps 1 thru 7 above, or consolidated into a single round of mutagenesis. For example, Step 2 above could be performed for each mutation, then the products of both mutation amplifications could be combined with the product of Step 3 and 4, and the procedure followed as described.

### Example 9

### Alternative Methods of Detecting Polymorphisms Encompassed by the Present Invention

### A. Preparation of Samples

Polymorphisms are detected in a target nucleic acid from an individual being analyzed. For assay of genomic DNA, virtually any biological sample (other than pure red blood cells) is suitable. For example, convenient tissue samples include whole blood, semen, saliva, tears, urine, fecal material, sweat, buccal, skin and hair. For assay of cDNA or mRNA, the tissue sample must be obtained from an organ in which the target nucleic acid is expressed. For example, if the target nucleic acid is a cytochrome P450, the liver is a suitable source.

Many of the methods described below require amplification of DNA from target samples. This can be accomplished by e.g., PCR. See generally PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, (1991); PCR (eds. McPherson et al., IRL Press, Oxford); and U.S. Pat. No. 4,683,202.

Other suitable amplification methods include the ligase chain reaction (LCR) (see Wu and Wallace, Genomics 4:560 (1989), Landegren et al., Science 241:1077 (1988), transcription amplification (Kwoh et al., Proc. Natl. Acad. Sci. USA 86, 1173 (1989), and self-sustained sequence replication (Guatelli et al., Proc. Nat. Acad. Sci. USA, 87:1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

Additional methods of amplification are known in the art or are described elsewhere herein.

### B. Detection of Polymorphisms in Target DNA

There are two distinct types of analysis of target DNA for detecting polymorphisms. The first type of analysis, sometimes referred to as de novo characterization, is carried out to identify polymorphic sites not previously characterized (i.e., to identify new polymorphisms). This analysis compares target sequences in different individuals to identify points of variation, i.e., polymorphic sites. By analyzing groups of individuals representing the greatest ethnic diversity among humans and greatest breed and species variety in plants and animals, patterns characteristic of the most common alleles/haplotypes of the locus can be identified, and the frequencies of such alleles/haplotypes in the population can be determined. Additional allelic frequencies can be determined for subpopulations characterized by criteria such as geography, race, or gender. The de novo identification of polymorphisms of the invention is described in the Examples section.

The second type of analysis determines which form(s) of a characterized (known) polymorphism are present in individuals under test. Additional methods of analysis are known in the art or are described elsewhere herein.

### 1. Allele-Specific Probes

The design and use of allele-specific probes for analyzing polymorphisms is described by e.g., Saiki et al., Nature 324,163-166 (1986); Dattagupta, EP 235,726, Saiki, WO 89/11548. Allele-specific probes can be designed that hybridize to a segment of target DNA from one individual but do not hybridize to the corresponding segment from another individual due to the presence of different polymorphic forms in the respective segments from the two individuals. Hybridization conditions should be sufficiently stringent that there is a significant difference in hybridization intensity between alleles, and preferably an essentially binary response, whereby a probe hybridizes to only one of the alleles. Some probes are designed to hybridize to a segment of target DNA such that the polymorphic site aligns with a central position (e.g., in a 15-mer at the 7 position; in a 16-mer, at either the 8 or 9 position) of the probe. This design of probe achieves good discrimination in hybridization between different allelic forms.

Allele-specific probes are often used in pairs, one member of a pair showing a perfect match to a reference form of a target sequence and the other member showing a perfect match to a variant form. Several pairs of probes can then be immobilized on the same support for simultaneous analysis of multiple polymorphisms within the same target sequence.

### 2. Tiling Arrays

The polymorphisms can also be identified by hybridization to nucleic acid arrays, some examples of which are described in WO 95/11995. The same arrays or different arrays can be used for analysis of characterized polymorphisms. WO 95/11995 also describes sub arrays that are optimized for detection of a variant form of a precharacterized polymorphism. Such a sub array contains probes designed to be complementary to a second reference sequence, which is an allelic variant of the first reference sequence. The second group of probes is designed by the same principles as described, except that the probes exhibit complementarity to the second reference sequence. The inclusion of a second group (or further groups) can be particularly useful for analyzing short subsequences of the primary reference sequence in which multiple mutations are expected to occur within a short distance commensurate with the length of the probes (e.g., two or more mutations within 9 to bases).

### 3. Allele-Specific Primers

An allele-specific primer hybridizes to a site on target DNA overlapping a polymorphism and only primes amplification of an allelic form to which the primer exhibits perfect complementarity. See Gibbs, Nucleic Acid Res. 17,2427-2448 (1989). This primer is used in conjunction with a second primer which hybridizes at a distal site. Amplification proceeds from the two primers, resulting in a detectable product which indicates the particular allelic form is present. A control is usually performed with a second pair of primers, one of which shows a single base mismatch at the polymorphic site and the other of which exhibits perfect complementarity to a distal site. The single-base mismatch prevents amplification and no detectable product is formed. The method works best when the mismatch is included in the 3′-most position of the oligonucleotide aligned with the polymorphism because this position is most destabilizing elongation from the primer (see, e.g., WO 93/22456).

### 4. Direct-Sequencing

The direct analysis of the sequence of polymorphisms of the present invention can be accomplished using either the dideoxy chain termination method or the Maxam-Gilbert method (see Sambrook et al., Molecular Cloning, A Laboratory Manual (2nd Ed., CSHP, New York 1989); Zyskind et al., Recombinant DNA Laboratory Manual, (Acad. Press, 1988)).

### 5. Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. Erlich, ed., PCR Technology. Principles and Applications for DNA Amplification, (W. H. Freeman and Co, New York, 1992), Chapter 7.

### 6. Single-Strand Conformation Polymorphism Analysis

Alleles of target sequences can be differentiated using single-strand conformation polymorphism analysis, which identifies base differences by alteration in electrophoretic migration of single stranded PCR products, as described in Orita et al., Proc. Nat. Acad. Sci. 86,2766-2770 (1989). Amplified PCR products can be generated as described above, and heated or otherwise denatured, to form single stranded amplification products. Single-stranded nucleic acids may refold or form secondary structures which are partially dependent on the base sequence. The different electrophoretic mobilities of single-stranded amplification products can be related to base-sequence differences between alleles of target sequences.

### 7. Single Base Extension

An alternative method for identifying and analyzing polymorphisms is based on single-base extension (SBE) of a fluorescently-labeled primer coupled with fluorescence resonance energy transfer (FRET) between the label of the added base and the label of the primer. Typically, the method, such as that described by Chen et al., (PNAS 94:10756-61 (1997), uses a locus-specific oligonucleotide primer labeled on the 5′ terminus with 5-carboxyfluorescein (FAM). This labeled primer is designed so that the 3′ end is immediately adjacent to the polymorphic site of interest. The labeled primer is hybridized to the locus, and single base extension of the labeled primer is performed with fluorescently-labeled dideoxyribonucleotides (ddNTPs) in dye-terminator sequencing fashion. An increase in fluorescence of the added ddNTP in response to excitation at the wavelength of the labeled primer is used to infer the identity of the added nucleotide.

### Example 10

### Method of Assessing the Ability of the Novel Organic Anion Transport and Multi-Drug Resistant Proteins of the Present Invention to Serve as Organic Anion Transportors

The activity of the the novel organic anion transport polypeptides of the present invention, specifically the OATP2 allelic variants of the present invention, may be measured using an assay using a variety of assays known in the art for organic anion transporters, but preferably the assay provided in PCT International Publication No. WO0071566. OF particular importance would be to test each variants ability to transport pravastatin, DHEAS, and taurocholate relative to the wild-type OATP2 protein. Briefly, 293c18 cells, an HEK293 derivative, are transiently transfected with the OATP2 expression vector pCEPOATP2, or the pCEP4 vector alone (MOCK) and the transport of [³H]-labeled substrates is determined 24 hours later. Specific uptake of [³H]-pravastatin and [³H]-DHEAS, and [³H]-taurocholate may then be observed in cells transfected with OATP2 but not in the mock transfected cells.

### Example 11

### Bacterial Expression of a Polypeptide

A polynucleotide encoding a polypeptide of the present invention is amplified using PCR oligonucleotide primers corresponding to the 5′ and 3′ ends of the DNA sequence, as outlined in the Examples above or otherwise known in the art, to synthesize insertion fragments. The primers used to amplify the cDNA insert should preferably contain restriction sites, such as BamHI and XbaI, at the 5′ end of the primers in order to clone the amplified product into the expression vector. For example, BamHI and XbaI correspond to the restriction enzyme sites on the bacterial expression vector pQE-9. (Qiagen, Inc., Chatsworth, Calif.). This plasmid vector encodes antibiotic resistance (Ampr), a bacterial origin of replication (ori), an IPTG-regulatable promoter/operator (P/O), a ribosome binding site (RBS), a 6-histidine tag (6-His), and restriction enzyme cloning sites.

The pQE-9 vector is digested with BamHI and XbaI and the amplified fragment is ligated into the pQE-9 vector maintaining the reading frame initiated at the bacterial RBS. The ligation mixture is then used to transform the E. coli strain M15/rep4 (Qiagen, Inc.) which contains multiple copies of the plasmid pREP4, that expresses the lacI repressor and also confers kanamycin resistance (Kanr). Transformants are identified by their ability to grow on LB plates and ampicillin/kanamycin resistant colonies are selected. Plasmid DNA is isolated and confirmed by restriction analysis.

Clones containing the desired constructs are grown overnight (O/N) in liquid culture in LB media supplemented with both Amp (100 ug/ml) and Kan (25 ug/ml). The O/N culture is used to inoculate a large culture at a ratio of 1:100 to 1:250. The cells are grown to an optical density 600 (O.D.600) of between 0.4 and 0.6. IPTG (Isopropyl-B-D-thiogalacto pyranoside) is then added to a final concentration of 1 mM. IPTG induces by inactivating the lacd repressor, clearing the P/O leading to increased gene expression.

Cells are grown for an extra 3 to 4 hours. Cells are then harvested by centrifugation (20 mins at 6000×g). The cell pellet is solubilized in the chaotropic agent 6 Molar Guanidine HCl by stirring for 3-4 hours at 4 degree C. The cell debris is removed by centrifugation, and the supernatant containing the polypeptide is loaded onto a nickel-nitrilo-tri-acetic acid (“Ni-NTA”) affinity resin column (available from QIAGEN, Inc., supra). Proteins with a 6×His tag bind to the Ni-NTA resin with high affinity and can be purified in a simple one-step procedure (for details see: The QIAexpressionist (1995) QIAGEN, Inc., supra).

Briefly, the supernatant is loaded onto the column in 6 M guanidine-HCl, pH 8, the column is first washed with 10 volumes of 6 M guanidine-HCl, pH 8, then washed with 10 volumes of 6 M guanidine-HCl pH 6, and finally the polypeptide is eluted with 6 M guanidine-HCl, pH 5.

The purified protein is then renatured by dialyzing it against phosphate-buffered saline (PBS) or 50 mM Na-acetate, pH 6 buffer plus 200 mM NaCl. Alternatively, the protein can be successfully refolded while immobilized on the Ni-NTA column. The recommended conditions are as follows: renature using a linear 6M-1M urea gradient in 500 mM NaCl, 20% glycerol, 20 mM Tris/HCl pH 7.4, containing protease inhibitors. The renaturation should be performed over a period of 1.5 hours or more. After renaturation the proteins are eluted by the addition of 250 mM imidazole. Imidazole is removed by a final dialyzing step against PBS or 50 mM sodium acetate pH 6 buffer plus 200 mM NaCl. The purified protein is stored at 4 degree C or frozen at −80 degree C.

### Example 12

### Purification of a Polypeptide from an Inclusion Body

The following alternative method can be used to purify a polypeptide expressed in E coli when it is present in the form of inclusion bodies. Unless otherwise specified, all of the following steps are conducted at 4-10 degree C.

Upon completion of the production phase of the E. coli fermentation, the cell culture is cooled to 4-10 degree C and the cells harvested by continuous centrifugation at 15,000 rpm (Heraeus Sepatech). On the basis of the expected yield of protein per unit weight of cell paste and the amount of purified protein required, an appropriate amount of cell paste, by weight, is suspended in a buffer solution containing 100 mM Tris, 50 mM EDTA, pH 7.4. The cells are dispersed to a homogeneous suspension using a high shear mixer.

The cells are then lysed by passing the solution through a microfluidizer (Microfluidics, Corp. or APV Gaulin, Inc.) twice at 4000-6000 psi. The homogenate is then mixed with NaCl solution to a final concentration of 0.5 M NaCl, followed by centrifugation at 7000×g for 15 min. The resultant pellet is washed again using 0.5M NaCl, 100 mM Tris, 50 mM EDTA, pH 7.4.

The resulting washed inclusion bodies are solubilized with 1.5 M guanidine hydrochloride (GuHCl) for 2-4 hours. After 7000×g centrifugation for 15 min., the pellet is discarded and the polypeptide containing supernatant is incubated at 4 degree C overnight to allow further GuHCl extraction.

Following high speed centrifugation (30,000×g) to remove insoluble particles, the GuHCl solubilized protein is refolded by quickly mixing the GuHCl extract with 20 volumes of buffer containing 50 mM sodium, pH 4.5, 150 mM NaCl, 2 mM EDTA by vigorous stirring. The refolded diluted protein solution is kept at 4 degree C without mixing for 12 hours prior to further purification steps.

To clarify the refolded polypeptide solution, a previously prepared tangential filtration unit equipped with 0.16 um membrane filter with appropriate surface area (e.g., Filtron), equilibrated with 40 mM sodium acetate, pH 6.0 is employed. The filtered sample is loaded onto a cation exchange resin (e.g., Poros HS-50, Perceptive Biosystems). The column is washed with 40 mM sodium acetate, pH 6.0 and eluted with 250 mM, 500 mM, 1000 mM, and 1500 mM NaCl in the same buffer, in a stepwise manner. The absorbance at 280 nm of the effluent is continuously monitored. Fractions are collected and further analyzed by SDS-PAGE.

Fractions containing the polypeptide are then pooled and mixed with 4 volumes of water. The diluted sample is then loaded onto a previously prepared set of tandem columns of strong anion (Poros HQ-50, Perceptive Biosystems) and weak anion (Poros CM-20, Perceptive Biosystems) exchange resins. The columns are equilibrated with 40 mM sodium acetate, pH 6.0. Both columns are washed with 40 mM sodium acetate, pH 6.0, 200 mM NaCl. The CM-20 column is then eluted using a 10 column volume linear gradient ranging from 0.2 M NaCl, 50 mM sodium acetate, pH 6.0 to 1.0 M NaCl, 50 mM sodium acetate, pH 6.5. Fractions are collected under constant A280 monitoring of the effluent. Fractions containing the polypeptide (determined, for instance, by 16% SDS-PAGE) are then pooled.

The resultant polypeptide should exhibit greater than 95% purity after the above refolding and purification steps. No major contaminant bands should be observed from Coomassie blue stained 16% SDS-PAGE gel when 5 ug of purified protein is loaded. The purified protein can also be tested for endotoxin/LPS contamination, and typically the LPS content is less than 0.1 ng/ml according to LAL assays.

### Example 13

### Cloning and Expression of a Polypeptide in a Baculovirus Expression System

In this example, the plasmid shuttle vector pAc373 is used to insert a polynucleotide into a baculovirus to express a polypeptide. A typical baculovirus expression vector contains the strong polyhedrin promoter of the Autographa californica nuclear polyhedrosis virus (AcMNPV) followed by convenient restriction sites, which may include, for example BamHI, Xba I and Asp718. The polyadenylation site of the simian virus 40 (“SV40”) is often used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate a viable virus that express the cloned polynucleotide.

Many other baculovirus vectors can be used in place of the vector above, such as pVL941 and pAcIM1, as one skilled in the art would readily appreciate, as long as the construct provides appropriately located signals for transcription, translation, secretion and the like, including a signal peptide and an in-frame AUG as required. Such vectors are described, for instance, in Luckow et al., Virology 170:31-39 (1989).

A polynucleotide encoding a polypeptide of the present invention is amplified using PCR oligonucleotide primers corresponding to the 5′ and 3′ ends of the DNA sequence, as outlined in the Examples above or otherwise known in the art, to synthesize insertion fragments. The primers used to amplify the cDNA insert should preferably contain restriction sites at the 5′ end of the primers in order to clone the amplified product into the expression vector. Specifically, the cDNA sequence contained in the deposited clone, including the AUG initiation codon and the naturally associated leader sequence identified elsewhere herein (if applicable), is amplified using the PCR protocol described herein. If the naturally occurring signal sequence is used to produce the protein, the vector used does not need a second signal peptide. Alternatively, the vector can be modified to include a baculovirus leader sequence, using the standard methods described in Summers et al., “A Manual of Methods for Baculovirus Vectors and Insect Cell Culture Procedures” Texas Agricultural Experimental Station Bulletin No. 1555 (1987).

The amplified fragment is isolated from a 1% agarose gel using a commercially available kit (“Geneclean” BIO 101 Inc., La Jolla, Calif.). The fragment then is digested with appropriate restriction enzymes and again purified on a 1% agarose gel.

The plasmid is digested with the corresponding restriction enzymes and optionally, can be dephosphorylated using calf intestinal phosphatase, using routine procedures known in the art. The DNA is then isolated from a 1% agarose gel using a commercially available kit (“Geneclean” BIO 101 Inc., La Jolla, Calif.).

The fragment and the dephosphorylated plasmid are ligated together with T4 DNA ligase. E. coli HB101 or other suitable E. coli hosts such as XL-1 Blue (Stratagene Cloning Systems, La Jolla, Calif.) cells are transformed with the ligation mixture and spread on culture plates. Bacteria containing the plasmid are identified by digesting DNA from individual colonies and analyzing the digestion product by gel electrophoresis. The sequence of the cloned fragment is confirmed by DNA sequencing.

Five ug of a plasmid containing the polynucleotide is co-transformed with 1.0 ug of a commercially available linearized baculovirus DNA (“BaculoGoldtm baculovirus DNA”, Pharmingen, San Diego, Calif.), using the lipofection method described by Felgner et al., Proc. Natl. Acad. Sci. USA 84:7413-7417 (1987). One ug of BaculoGoldtm virus DNA and 5 ug of the plasmid are mixed in a sterile well of a microtiter plate containing 50 ul of serum-free Grace's medium (Life Technologies Inc., Gaithersburg, Md.). Afterwards, 10 ul Lipofectin plus 90 ul Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Grace's medium without serum. The plate is then incubated for 5 hours at 27 degrees C. The transfection solution is then removed from the plate and 1 ml of Grace's insect medium supplemented with 10% fetal calf serum is added. Cultivation is then continued at 27 degrees C for four days.

After four days the supernatant is collected and a plaque assay is performed, as described by Summers and Smith, supra. An agarose gel with “Blue Gal” (Life Technologies Inc., Gaithersburg) is used to allow easy identification and isolation of gal-expressing clones, which produce blue-stained plaques. (A detailed description of a “plaque assay” of this type can also be found in the user's guide for insect cell culture and baculovirology distributed by Life Technologies Inc., Gaithersburg, page 9-10.) After appropriate incubation, blue stained plaques are picked with the tip of a micropipettor (e.g., Eppendorf). The agar containing the recombinant viruses is then resuspended in a microcentrifuge tube containing 200 ul of Grace's medium and the suspension containing the recombinant baculovirus is used to infect Sf9 cells seeded in 35 mm dishes. Four days later the supernatants of these culture dishes are harvested and then they are stored at 4 degree C.

To verify the expression of the polypeptide, Sf9 cells are grown in Grace's medium supplemented with 10% heat-inactivated FBS. The cells are infected with the recombinant baculovirus containing the polynucleotide at a multiplicity of infection (“MOI”) of about 2. If radiolabeled proteins are desired, 6 hours later the medium is removed and is replaced with SF900 II medium minus methionine and cysteine (available from Life Technologies Inc., Rockville, Md.). After 42 hours, 5 uCi of 35S-methionine and 5 uCi 35S-cysteine (available from Amersham) are added. The cells are further incubated for 16 hours and then are harvested by centrifugation. The proteins in the supernatant as well as the intracellular proteins are analyzed by SDS-PAGE followed by autoradiography (if radiolabeled).

Microsequencing of the amino acid sequence of the amino terminus of purified protein may be used to determine the amino terminal sequence of the produced protein.

### Example 14

### Expression of a Polypeptide in Mammalian Cells

The polypeptide of the present invention can be expressed in a mammalian cell. A typical mammalian expression vector contains a promoter element, which mediates the initiation of transcription of mRNA, a protein coding sequence, and signals required for the termination of transcription and polyadenylation of the transcript. Additional elements include enhancers, Kozak sequences and intervening sequences flanked by donor and acceptor sites for RNA splicing. Highly efficient transcription is achieved with the early and late promoters from SV40, the long terminal repeats (LTRs) from Retroviruses, e.g., RSV, HTLVI, HIVI and the early promoter of the cytomegalovirus (CMV). However, cellular elements can also be used (e.g., the human actin promoter).

Suitable expression vectors for use in practicing the present invention include, for example, vectors such as pSVL and pMSG (Pharmacia, Uppsala, Sweden), pRSVcat (ATCC 37152), pSV2dhfr (ATCC 37146), pBC12MI (ATCC 67109), pCMVSport 2.0, and pCMVSport 3.0. Mammalian host cells that could be used include, human Hela, 293, H9 and Jurkat cells, mouse NIH3T3 and C127 cells, Cos 1, Cos 7 and CV1, quail QC1-3 cells, mouse L cells and Chinese hamster ovary (CHO) cells.

Alternatively, the polypeptide can be expressed in stable cell lines containing the polynucleotide integrated into a chromosome. The co-transformation with a selectable marker such as dhfr, gpt, neomycin, hygromycin allows the identification and isolation of the transformed cells.

The transformed gene can also be amplified to express large amounts of the encoded protein. The DHFR (dihydrofolate reductase) marker is useful in developing cell lines that carry several hundred or even several thousand copies of the gene of interest. (See, e.g., Alt, F. W., et al., J. Biol. Chem. 253:1357-1370 (1978); Hamlin, J. L. and Ma, C., Biochem. et Biophys. Acta, 1097:107-143 (1990); Page, M. J. and Sydenham, M. A., Biotechnology 9:64-68 (1991).) Another useful selection marker is the enzyme glutamine synthase (GS) (Murphy et al., Biochem J. 227:277-279 (1991); Bebbington et al., Bio/Technology 10:169-175 (1992). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of proteins.

A polynucleotide of the present invention is amplified according to the protocol outlined in herein. If the naturally occurring signal sequence is used to produce the protein, the vector does not need a second signal peptide. Alternatively, if the naturally occurring signal sequence is not used, the vector can be modified to include a heterologous signal sequence. (See, e.g., WO 96/34891.) The amplified fragment is isolated from a 1% agarose gel using a commercially available kit (“Geneclean” BIO 101 Inc., La Jolla, Calif.). The fragment then is digested with appropriate restriction enzymes and again purified on a 1% agarose gel.

The amplified fragment is then digested with the same restriction enzyme and purified on a 1% agarose gel. The isolated fragment and the dephosphorylated vector are then ligated with T4 DNA ligase. E. coli HB101 or XL-1 Blue cells are then transformed and bacteria are identified that contain the fragment inserted into plasmid pC6 using, for instance, restriction enzyme analysis.

Chinese hamster ovary cells lacking an active DHFR gene is used for transformation. Five μg of an expression plasmid is cotransformed with 0.5 ug of the plasmid pSVneo using lipofectin (Felgner et al., supra). The plasmid pSV2-neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of methotrexate plus 1 mg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 uM, 2 uM, 5 uM, 10 mM, 20 mM). The same procedure is repeated until clones are obtained which grow at a concentration of 100 -200 uM. Expression of the desired gene product is analyzed, for instance, by SDS-PAGE and Western blot or by reversed phase HPLC analysis.

### Example 15

### Protein Fusions

The polypeptides of the present invention are preferably fused to other proteins. These fusion proteins can be used for a variety of applications. For example, fusion of the present polypeptides to His-tag, HA-tag, protein A, IgG domains, and maltose binding protein facilitates purification. (See Example described herein; see also EP A 394,827; Traunecker, et al., Nature 331:84-86 (1988).) Similarly, fusion to IgG-1, IgG-3, and albumin increases the half-life time in vivo. Nuclear localization signals fused to the polypeptides of the present invention can target the protein to a specific subcellular localization, while covalent heterodimer or homodimers can increase or decrease the activity of a fusion protein. Fusion proteins can also create chimeric molecules having more than one function. Finally, fusion proteins can increase solubility and/or stability of the fused protein compared to the non-fused protein. All of the types of fusion proteins described above can be made by modifying the following protocol, which outlines the fusion of a polypeptide to an IgG molecule.

Briefly, the human Fc portion of the IgG molecule can be PCR amplified, using primers that span the 5′ and 3′ ends of the sequence described below. These primers also should have convenient restriction enzyme sites that will facilitate cloning into an expression vector, preferably a mammalian expression vector. Note that the polynucleotide is cloned without a stop codon, otherwise a fusion protein will not be produced.

The naturally occurring signal sequence may be used to produce the protein (if applicable). Alternatively, if the naturally occurring signal sequence is not used, the vector can be modified to include a heterologous signal sequence. (See, e.g., WO 96/34891 and/or U.S. Pat. No. 6,066,781, supra.)

Human IgG Fc region:

(SEQ ID NO:606)

GGGATCCGGAGCCCAAATCTTCTGACAAAACTCACACATGCCCACCGTGC
CCAGCACCTGAATTCGAGGGTGCACCGTCAGTCTTCCTCTTCCCCCCAAA
ACCCAAGGACACCCTCATGATCTCCCGGACTCCTGAGGTCACATGCGTGG
TGGTGGACGTAAGCCACGAAGACCCTGAGGTCAAGTTCAACTGGTACGTG
GACGGCGTGGAGGTGCATAATGCCAAGACAAAGCCGCGGGAGGAGCAGTA
CAACAGCACGTACCGTGTGGTCAGCGTCCTCACCGTCCTGCACCAGGACT
GGCTGAATGGCAAGGAGTACAAGTGCAAGGTCTCCAACAAAGCCCTCCCA
ACCCCCATCGAGAAAACCATCTCCAAAGCCAAAGGGCAGCCCCGAGAACC
ACAGGTGTACACCCTGCCCCCATCCCGGGATGAGCTGACCAAGAACCAGG
TCAGCCTGACCTGCCTGGTCAAAGGCTTCTATCCAAGCGACATCGCCGTG
GAGTGGGAGAGCAATGGGCAGCCGGAGAACAACTACAAGACCACGCCTCC
CGTGCTGGACTCCGACGGCTCCTTCTTCCTCTACAGCAAGCTCACCGTGG
ACAAGAGCAGGTGGCAGCAGGGGAACGTCTTCTCATGCTCCGTGATGCAT
GAGGCTCTGCACAACCACTACACGCAGAAGAGCCTCTCCCTGTCTCCGGG
TAAATGAGTCCGACGGCCGCGACTCTAGAGGAT

### Example 16

### Production of an Antibody from a Polypeptide

The antibodies of the present invention can be prepared by a variety of methods. (See, Current Protocols, Chapter 2.) As one example of such methods, cells expressing a polypeptide of the present invention are administered to an animal to induce the production of sera containing polyclonal antibodies. In a preferred method, a preparation of the protein is prepared and purified to render it substantially free of natural contaminants. Such a preparation is then introduced into an animal in order to produce polyclonal antisera of greater specific activity.

In the most preferred method, the antibodies of the present invention are monoclonal antibodies (or protein binding fragments thereof). Such monoclonal antibodies can be prepared using hybridoma technology. (Kohler et al., Nature 256:495 (1975); Kohler et al., Eur. J. Immunol. 6:511 (1976); Kohler et al., Eur. J. Immunol. 6:292 (1976); Hammerling et al., in: Monoclonal Antibodies and T-Cell Hybridomas, Elsevier, N.Y., pp. 563-681 (1981).) In general, such procedures involve immunizing an animal (preferably a mouse) with polypeptide or, more preferably, with a polypeptide-expressing cell. Such cells may be cultured in any suitable tissue culture medium; however, it is preferable to culture cells in Earle's modified Eagle's medium supplemented with 10% fetal bovine serum (inactivated at about 56 degrees C), and supplemented with about 10 g/l of nonessential amino acids, about 1,000 U/ml of penicillin, and about 100 ug/ml of streptomycin.

The splenocytes of such mice are extracted and fused with a suitable myeloma cell line. Any suitable myeloma cell line may be employed in accordance with the present invention; however, it is preferable to employ the parent myeloma cell line (SP20), available from the ATCC. After fusion, the resulting hybridoma cells are selectively maintained in HAT medium, and then cloned by limiting dilution as described by Wands et al. (Gastroenterology 80:225-232 (1981).) The hybridoma cells obtained through such a selection are then assayed to identify clones which secrete antibodies capable of binding the polypeptide.

Alternatively, additional antibodies capable of binding to the polypeptide can be produced in a two-step procedure using anti-idiotypic antibodies. Such a method makes use of the fact that antibodies are themselves antigens, and therefore, it is possible to obtain an antibody that binds to a second antibody. In accordance with this method, protein specific antibodies are used to immunize an animal, preferably a mouse. The splenocytes of such an animal are then used to produce hybridoma cells, and the hybridoma cells are screened to identify clones that produce an antibody whose ability to bind to the protein-specific antibody can be blocked by the polypeptide. Such antibodies comprise anti-idiotypic antibodies to the protein-specific antibody and can be used to immunize an animal to induce formation of further protein-specific antibodies.

It will be appreciated that Fab and F(ab′)2 and other fragments of the antibodies of the present invention may be used according to the methods disclosed herein. Such fragments are typically produced by proteolytic cleavage, using enzymes such as papain (to produce Fab fragments) or pepsin (to produce F(ab′)2 fragments). Alternatively, protein-binding fragments can be produced through the application of recombinant DNA technology or through synthetic chemistry.

For in vivo use of antibodies in humans, it may be preferable to use “humanized” chimeric monoclonal antibodies. Such antibodies can be produced using genetic constructs derived from hybridoma cells producing the monoclonal antibodies described above. Methods for producing chimeric antibodies are known in the art. (See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).)

Moreover, in another preferred method, the antibodies directed against the polypeptides of the present invention may be produced in plants. Specific methods are disclosed in U.S. Pat. Nos. 5,959,177, and 6,080,560, which are hereby incorporated in their entirety herein. The methods not only describe methods of expressing antibodies, but also the means of assembling foreign multimeric proteins in plants (i.e., antibodies, etc,), and the subsequent secretion of such antibodies from the plant.

### Example 17

### Alteration of Protein Glycosylation Sites to Enhance Characteristics of Polypeptides of the Invention

Many eukaryotic cell surface and proteins are post-translationally processed to incorporate N-linked and O-linked carbohydrates (Kornfeld and Kornfeld (1985) Annu. Rev. Biochem. 54:631-64; Rademacher et al., (1988) Annu. Rev. Biochem. 57:785-838). Protein glycosylation is thought to serve a variety of functions including: augmentation of protein folding, inhibition of protein aggregation, regulation of intracellular trafficking to organelles, increasing resistance to proteolysis, modulation of protein antigenicity, and mediation of intercellular adhesion (Fieldler and Simons (1995) Cell, 81:309-312; Helenius (1994) Mol. Biol. Of the Cell 5:253-265; Olden et al., (1978) Cell, 13:461-473; Caton et al., (1982) Cell, 37:417-427; Alexamnder and Elder (1984), Science, 226:1328-1330; and Flack et al., (1994), J. Biol. Chem., 269:14015-14020). In higher organisms, the nature and extent of glycosylation can markedly affect the circulating half-life and bio-availability of proteins by mechanisms involving receptor mediated uptake and clearance (Ashwell and Morrell, (1974), Adv. Enzymol., 41:99-128; Ashwell and Harford (1982), Ann. Rev. Biochem., 51:531-54). Receptor systems have been identified that are thought to play a major role in the clearance of serum proteins through recognition of various carbohydrate structures on the glycoproteins (Stockert (1995), Physiol. Rev., 75:591-609; Kery et al., (1992), Arch. Biochem. Biophys., 298:49-55). Thus, production strategies resulting in incomplete attachment of terminal sialic acid residues might provide a means of shortening the bioavailability and half-life of glycoproteins. Conversely, expression strategies resulting in saturation of terminal sialic acid attachment sites might lengthen protein bioavailability and half-life.

In the development of recombinant glycoproteins for use as pharmaceutical products, for example, it has been speculated that the pharmacodynamics of recombinant proteins can be modulated by the addition or deletion of glycosylation sites from a glycoproteins primary structure (Berman and Lasky (1985a) Trends in Biotechnol., 3:51-53). However, studies have reported that the deletion of N-linked glycosylation sites often impairs intracellular transport and results in the intracellular accumulation of glycosylation site variants (Machamer and Rose (1988), J. Biol Chem., 263:5955-5960; Gallagher et al., (1992), J. Virology., 66:7136-7145; Collier et al., (1993), Biochem., 32:7818-7823; Claffey et al., (1995) Biochemica et Biophysica Acta, 1246:1-9; Dube et al., (1988), J. Biol. Chem. 263:17516-17521). While glycosylation site variants of proteins can be expressed intracellularly, it has proved difficult to recover useful quantities from growth conditioned cell culture medium.

Moreover, it is unclear to what extent a glycosylation site in one species will be recognized by another species glycosylation machinery. Due to the importance of glycosylation in protein metabolism, particularly the secretion and/or expression of the protein, whether a glycosylation signal is recognized may profoundly determine a proteins ability to be expressed, either endogenously or recombinately, in another organism (i.e., expressing a human protein in E.coli, yeast, or viral organisms; or an E.coli, yeast, or viral protein in human, etc.). Thus, it may be desirable to add, delete, or modify a glycosylation site, and possibly add a glycosylation site of one species to a protein of another species to improve the proteins functional, bioprocess purification, and/or structural characteristics (e.g., a polypeptide of the present invention).

A number of methods may be employed to identify the location of glycosylation sites within a protein. One preferred method is to run the translated protein sequence through the PROSITE computer program (Swiss Institute of Bioinformatics). Once identified, the sites could be systematically deleted, or impaired, at the level of the DNA using mutagenesis methodology known in the art and available to the skilled artisan, Preferably using PCR-directed mutagenesis (See Maniatis, Molecular Cloning: A Laboratory Manual, Cold Spring Harbor Press, Cold Spring, N.Y. (1982)). Similarly, glycosylation sites could be added, or modified at the level of the DNA using similar methods, preferably PCR methods (See, Maniatis, supra). The results of modifying the glycosylation sites for a particular protein (e.g., solubility, secretion potential, activity, aggregation, proteolytic resistance, etc.) could then be analyzed using methods know in the art.

The skilled artisan would acknowledge the existence of other computer algorithms capable of predicting the location of glycosylation sites within a protein. For example, the Motif computer program (Genetics Computer Group suite of programs) provides this function, as well.

### Example 18

### Method of Enhancing the Biological Activity/Functional Characteristics of Invention Through Molecular Evolution

Although many of the most biologically active proteins known are highly effective for their specified function in an organism, they often possess characteristics that make them undesirable for transgenic, therapeutic, and/or industrial applications. Among these traits, a short physiological half-life is the most prominent problem, and is present either at the level of the protein, or the level of the proteins mRNA. The ability to extend the half-life, for example, would be particularly important for a proteins use in gene therapy, transgenic animal production, the bioprocess production and purification of the protein, and use of the protein as a chemical modulator among others. Therefore, there is a need to identify novel variants of isolated proteins possessing characteristics which enhance their application as a therapeutic for treating diseases of animal origin, in addition to the proteins applicability to common industrial and pharmaceutical applications.

Thus, one aspect of the present invention relates to the ability to enhance specific characteristics of invention through directed molecular evolution. Such an enhancement may, in a non-limiting example, benefit the inventions utility as an essential component in a kit, the inventions physical attributes such as its solubility, structure, or codon optimization, the inventions specific biological activity, including any associated enzymatic activity, the proteins enzyme kinetics, the proteins Ki, Kcat, Km, Vmax, Kd, protein-protein activity, protein-DNA binding activity, antagonist/inhibitory activity (including direct or indirect interaction), agonist activity (including direct or indirect interaction), the proteins antigenicity (e.g., where it would be desirable to either increase or decrease the antigenic potential of the protein), the immunogenicity of the protein, the ability of the protein to form dimers, trimers, or multimers with either itself or other proteins, the antigenic efficacy of the invention, including its subsequent use a preventative treatment for disease or disease states, or as an effector for targeting diseased genes. Moreover, the ability to enhance specific characteristics of a protein may also be applicable to changing the characterized activity of an enzyme to an activity completely unrelated to its initially characterized activity. Other desirable enhancements of the invention would be specific to each individual protein, and would thus be well known in the art and contemplated by the present invention.

Directed evolution is comprised of several steps. The first step is to establish a library of variants for the gene or protein of interest. The most important step is to then select for those variants that entail the activity you wish to identify. The design of the screen is essential since your screen should be selective enough to eliminate non-useful variants, but not so stringent as to eliminate all variants. The last step is then to repeat the above steps using the best variant from the previous screen. Each successive cycle, can then be tailored as necessary, such as increasing the stringency of the screen, for example.

Over the years, there have been a number of methods developed to introduce mutations into macromolecules. Some of these methods include, random mutagenesis, “error-prone” PCR, chemical mutagenesis, site-directed mutagenesis, and other methods well known in the art (for a comprehensive listing of current mutagenesis methods, see Maniatis, Molecular Cloning: A Laboratory Manual, Cold Spring Harbor Press, Cold Spring, N.Y. (1982)). Typically, such methods have been used, for example, as tools for identifying the core functional region(s) of a protein or the function of specific domains of a protein (if a multi-domain protein). However, such methods have more recently been applied to the identification of macromolecule variants with specific or enhanced characteristics.

Random mutagenesis has been the most widely recognized method to date. Typically, this has been carried out either through the use of “error-prone” PCR (as described in Moore, J., et al, Nature Biotechnology 14:458, (1996), or through the application of randomized synthetic oligonucleotides corresponding to specific regions of interest (as described by Derbyshire, K. M. et al, Gene, 46:145-152, (1986), and Hill, D E, et al, Methods Enzymol., 55:559-568, (1987). Both approaches have limits to the level of mutagenesis that can be obtained. However, either approach enables the investigator to effectively control the rate of mutagenesis. This is particularly important considering the fact that mutations beneficial to the activity of the enzyme are fairly rare. In fact, using too high a level of mutagenesis may counter or inhibit the desired benefit of a useful mutation.

While both of the aforementioned methods are effective for creating randomized pools of macromolecule variants, a third method, termed “DNA Shuffling”, or “sexual PCR” (W P C, Stemmer, PNAS, 91:10747, (1994)) has recently been elucidated. DNA shuffling has also been referred to as “directed molecular evolution”, “exon-shuffling”, “directed enzyme evolution”, “in vitro evolution”, and “artificial evolution”. Such reference terms are known in the art and are encompassed by the invention. This new, preferred, method apparently overcomes the limitations of the previous methods in that it not only propagates positive traits, but simultaneously eliminates negative traits in the resulting progeny.

DNA shuffling accomplishes this task by combining the principal of in vitro recombination, along with the method of “error-prone” PCR. In effect, you begin with a randomly digested pool of small fragments of your gene, created by Dnase I digestion, and then introduce said random fragments into an “error-prone” PCR assembly reaction. During the PCR reaction, the randomly sized DNA fragments not only hybridize to their cognate strand, but also may hybridize to other DNA fragments corresponding to different regions of the polynucleotide of interest—regions not typically accessible via hybridization of the entire polynucleotide. Moreover, since the PCR assembly reaction utilizes “error-prone” PCR reaction conditions, random mutations are introduced during the DNA synthesis step of the PCR reaction for all of the fragments—further diversifying the potential hybridization sites during the annealing step of the reaction.

A variety of reaction conditions could be utilized to carry-out the DNA shuffling reaction. However, specific reaction conditions for DNA shuffling are provided, for example, in PNAS, 91:10747, (1994). Briefly:

Prepare the DNA substrate to be subjected to the DNA shuffling reaction. Preparation may be in the form of simply purifying the DNA from contaminating cellular material, chemicals, buffers, oligonucleotide primers, deoxynucleotides, RNAs, etc., and may entail the use of DNA purification kits as those provided by Qiagen, Inc., or by the Promega, Corp., for example.

Once the DNA substrate has been purified, it would be subjected to Dnase I digestion. About 2-4 ug of the DNA substrate(s) would be digested with 0.0015 units of Dnase I (Sigma) per ul in 100 ul of 50 mM Tris-HCL, pH 7.4/1 mM MgCl2 for 10-20 min. at room temperature. The resulting fragments of 10-50bp could then be purified by running them through a 2% low-melting point agarose gel by electrophoresis onto DE81 ion-exchange paper (Whatmann) or could be purified using Microcon concentrators (Amicon) of the appropriate molecular weight cutoff, or could use oligonucleotide purification columns (Qiagen), in addition to other methods known in the art. If using DE81 ion-exchange paper, the 10-50 bp fragments could be eluted from said paper using 1M NaCl, followed by ethanol precipitation.

The resulting purified fragments would then be subjected to a PCR assembly reaction by re-suspension in a PCR mixture containing: 2 mM of each dNTP, 2.2 mM MgCl2, 50 mM KCl, 10 mM Tris.HCL, pH 9.0, and 0.1% Triton X-100, at a final fragment concentration of 10-30 ng/ul. No primers are added at this point. Taq DNA polymerase (Promega) would be used at 2.5 units per 100 ul of reaction mixture. A PCR program of 94 C for 60 s; 94 C for 30 s, 50-55 C for 30 s, and 72 C for 30 s using 30-45 cycles, followed by 72 C for 5 min using an MJ Research (Cambridge, Mass.) PTC-150 thermocycler. After the assembly reaction is completed, a 1:40 dilution of the resulting primerless product would then be introduced into a PCR mixture (using the same buffer mixture used for the assembly reaction) containing 0.8 um of each primer and subjecting this mixture to 15 cycles of PCR (using 94 C for 30 s, 50 C for 30 s, and 72 C for 30 s). The referred primers would be primers corresponding to the nucleic acid sequences of the polynucleotide(s) utilized in the shuffling reaction. Said primers could consist of modified nucleic acid base pairs using methods known in the art and referred to else where herein, or could contain additional sequences (i.e., for adding restriction sites, mutating specific base-pairs, etc.).

The resulting shuffled, assembled, and amplified product can be purified using methods well known in the art (e.g., Qiagen PCR purification kits) and then subsequently cloned using appropriate restriction enzymes.

Although a number of variations of DNA shuffling have been published to date, such variations would be obvious to the skilled artisan and are encompassed by the invention. The DNA shuffling method can also be tailored to the desired level of mutagenesis using the methods described by Zhao, et a]. (Nucl Acid Res., 25(6): 1307-1308, (1997).

As described above, once the randomized pool has been created, it can then be subjected to a specific screen to identify the variant possessing the desired characteristic(s). Once the variant has been identified, DNA corresponding to the variant could then be used as the DNA substrate for initiating another round of DNA shuffling. This cycle of shuffling, selecting the optimized variant of interest, and then re-shuffling, can be repeated until the ultimate variant is obtained. Examples of model screens applied to identify variants created using DNA shuffling technology may be found in the following publications: J. C., Moore, et al., J. Mol. Biol., 272:336-347, (1997), F. R., Cross, et al., Mol. Cell. Biol., 18:2923-2931, (1998), and A. Crameri., et al., Nat. Biotech., 15:436-438, (1997).

DNA shuffling has several advantages. First, it makes use of beneficial mutations. When combined with screening, DNA shuffling allows the discovery of the best mutational combinations and does not assume that the best combination contains all the mutations in a population. Secondly, recombination occurs simultaneously with point mutagenesis. An effect of forcing DNA polymerase to synthesize full-length genes from the small fragment DNA pool is a background mutagenesis rate. In combination with a stringent selection method, enzymatic activity has been evolved up to 16000 fold increase over the wild-type form of the enzyme. In essence, the background mutagenesis yielded the genetic variability on which recombination acted to enhance the activity.

A third feature of recombination is that it can be used to remove deleterious mutations. As discussed above, during the process of the randomization, for every one beneficial mutation, there may be at least one or more neutral or inhibitory mutations. Such mutations can be removed by including in the assembly reaction an excess of the wild-type random-size fragments, in addition to the random-size fragments of the selected mutant from the previous selection. During the next selection, some of the most active variants of the polynucleotide/polypeptide/enzyme, should have lost the inhibitory mutations.

Finally, recombination enables parallel processing. This represents a significant advantage since there are likely multiple characteristics that would make a protein more desirable (e.g. solubility, activity, etc.). Since it is increasingly difficult to screen for more than one desirable trait at a time, other methods of molecular evolution tend to be inhibitory. However, using recombination, it would be possible to combine the randomized fragments of the best representative variants for the various traits, and then select for multiple properties at once.

DNA shuffling can also be applied to the polynucleotides and polypeptides of the present invention to decrease their immunogenicity in a specified host. For example, a particular variant of the present invention may be created and isolated using DNA shuffling technology. Such a variant may have all of the desired characteristics, though may be highly immunogenic in a host due to its novel intrinsic structure. Specifically, the desired characteristic may cause the polypeptide to have a non-native structure which could no longer be recognized as a “self” molecule, but rather as a “foreign”, and thus activate a host immune response directed against the novel variant. Such a limitation can be overcome, for example, by including a copy of the gene sequence for a xenobiotic ortholog of the native protein in with the gene sequence of the novel variant gene in one or more cycles of DNA shuffling. The molar ratio of the ortholog and novel variant DNAs could be varied accordingly. Ideally, the resulting hybrid variant identified would contain at least some of the coding sequence which enabled the xenobiotic protein to evade the host immune system, and additionally, the coding sequence of the original novel variant that provided the desired characteristics.

Likewise, the invention encompasses the application of DNA shuffling technology to the evolution of polynucleotides and polypeptides of the invention, wherein one or more cycles of DNA shuffling include, in addition to the gene template DNA, oligonucleotides coding for known allelic sequences, optimized codon sequences, known variant sequences, known polynucleotide polymorphism sequences, known ortholog sequences, known homologue sequences, additional homologous sequences, additional non-homologous sequences, sequences from another species, and any number and combination of the above.

In addition to the described methods above, there are a number of related methods that may also be applicable, or desirable in certain cases. Representative among these are the methods discussed in PCT applications WO 98/31700, and WO 98/32845, which are hereby incorporated by reference. Furthermore, related methods can also be applied to the polynucleotide sequences of the present invention in order to evolve invention for creating ideal variants for use in gene therapy, protein engineering, evolution of whole cells containing the variant, or in the evolution of entire enzyme pathways containing polynucleotides of the invention as described in PCT applications WO 98/13485, WO 98/13487, WO 98/27230, WO 98/31837, and Crameri, A., et al., Nat. Biotech., 15:436-438, (1997), respectively.

Additional methods of applying “DNA Shuffling” technology to the polynucleotides and polypeptides of the present invention, including their proposed applications, may be found in U.S. Pat. No. 5,605,793; PCT Application No. WO 95/22625; PCT Application No. WO 97/20078; PCT Application No. WO 97/35966; and PCT Application No. WO 98/42832; PCT Application No. WO 00/09727 specifically provides methods for applying DNA shuffling to the identification of herbicide selective crops which could be applied to the polynucleotides and polypeptides of the present invention; additionally, PCT Application No. WO 00/12680 provides methods and compositions for generating, modifying, adapting, and optimizing polynucleotide sequences that confer detectable phenotypic properties on plant species; each of the above are hereby incorporated in their entirety herein for all purposes.

### Example 19

### Method of Determining Alterations in a Gene Corresponding to a Polynucleotide

RNA isolated from entire families or individual patients presenting with a phenotype of interest (such as a disease), is isolated. cDNA is then generated from these RNA samples using protocols known in the art. (See, Sambrook.) The cDNA is then used as a template for PCR, employing primers surrounding regions of interest, such as those sequences listed in the Sequence Listing and/or the Tables of the present invention. Suggested PCR conditions consist of 35 cycles at 95 degrees C for 30 seconds; 60-120 seconds at 52-58 degrees C; and 60-120 seconds at 70 degrees C, using buffer solutions described in Sidransky et al., Science 252:706 (1991).

PCR products are then sequenced using primers labeled at their 5′ end with T4 polynucleotide kinase, employing SequiTherm Polymerase. (Epicentre Technologies). The intron-exon borders of selected exons is also determined and genomic PCR products analyzed to confirm the results. PCR products harboring suspected mutations is then cloned and sequenced to validate the results of the direct sequencing.

PCR products are cloned into T-tailed vectors as described in Holton et al., Nucleic Acids Research, 19:1156 (1991) and sequenced with T7 polymerase (United States Biochemical). Affected individuals are identified by mutations not present in unaffected individuals.

Genomic rearrangements are also observed as a method of determining alterations in a gene corresponding to a polynucleotide. Genomic clones isolated according to the Examples provided herein or otherwise known in the art are nick-translated with digoxigenindeoxy-uridine 5′-triphosphate (Boehringer Manheim), and FISH performed as described in Johnson et al., Methods Cell Biol. 35:73-99 (1991). Hybridization with the labeled probe is carried out using a vast excess of human cot-1 DNA for specific hybridization to the corresponding genomic locus.

Chromosomes are counterstained with 4,6-diamino-2-phenylidole and propidium iodide, producing a combination of C- and R-bands. Aligned images for precise mapping are obtained using a triple-band filter set (Chroma Technology, Brattleboro, Vt.) in combination with a cooled charge-coupled device camera (Photometrics, Tucson, Ariz.) and variable excitation wavelength filters. (Johnson et al., Genet. Anal. Tech. Appl., 8:75 (1991).) Image collection, analysis and chromosomal fractional length measurements are performed using the ISee Graphical Program System. (Inovision Corporation, Durham, N.C.) Chromosome alterations of the genomic region hybridized by the probe are identified as insertions, deletions, and translocations. These alterations are used as a diagnostic marker for an associated disease.

### Example 20

### Method of Detecting Abnormal Levels of a Polypeptide in a Biological Sample

A polypeptide of the present invention can be detected in a biological sample, and if an increased or decreased level of the polypeptide is detected, this polypeptide is a marker for a particular phenotype. Methods of detection are numerous, and thus, it is understood that one skilled in the art can modify the following assay to fit their particular needs.

For example, antibody-sandwich ELISAs are used to detect polypeptides in a sample, preferably a biological sample. Wells of a microtiter plate are coated with specific antibodies, at a final concentration of 0.2 to 10 ug/ml. The antibodies are either monoclonal or polyclonal and are produced by the method described elsewhere herein. The wells are blocked so that non-specific binding of the polypeptide to the well is reduced.

The coated wells are then incubated for >2 hours at RT with a sample containing the polypeptide. Preferably, serial dilutions of the sample should be used to validate results. The plates are then washed three times with deionized or distilled water to remove unbounded polypeptide.

Next, 50 ul of specific antibody-alkaline phosphatase conjugate, at a concentration of 25-400 ng, is added and incubated for 2 hours at room temperature. The plates are again washed three times with deionized or distilled water to remove unbounded conjugate.

Add 75 ul of 4-methylumbelliferyl phosphate (MUP) or p-nitrophenyl phosphate (NPP) substrate solution to each well and incubate 1 hour at room temperature. Measure the reaction by a microtiter plate reader. Prepare a standard curve, using serial dilutions of a control sample, and plot polypeptide concentration on the X-axis (log scale) and fluorescence or absorbance of the Y-axis (linear scale). Interpolate the concentration of the polypeptide in the sample using the standard curve.

### Example 21

### Formulation

The invention also provides methods of treatment and/or prevention diseases, disorders, and/or conditions (such as, for example, any one or more of the diseases or disorders disclosed herein) by administration to a subject of an effective amount of a Therapeutic. By therapeutic is meant a polynucleotides or polypeptides of the invention (including fragments and variants), agonists or antagonists thereof, and/or antibodies thereto, in combination with a pharmaceutically acceptable carrier type (e.g., a sterile carrier).

The Therapeutic will be formulated and dosed in a fashion consistent with good medical practice, taking into account the clinical condition of the individual patient (especially the side effects of treatment with the Therapeutic alone), the site of delivery, the method of administration, the scheduling of administration, and other factors known to practitioners. The “effective amount” for purposes herein is thus determined by such considerations.

As a general proposition, the total pharmaceutically effective amount of the Therapeutic administered parenterally per dose will be in the range of about 1 ug/kg/day to 10 mg/kg/day of patient body weight, although, as noted above, this will be subject to therapeutic discretion. More preferably, this dose is at least 0.01 mg/kg/day, and most preferably for humans between about 0.01 and 1 mg/kg/day for the hormone. If given continuously, the Therapeutic is typically administered at a dose rate of about 1 ug/kg/hour to about 50 ug/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect.

Therapeutics can be administered orally, rectally, parenterally, intracisternally, intravaginally, intraperitoneally, topically (as by powders, ointments, gels, drops or transdermal patch), bucally, or as an oral or nasal spray. “Pharmaceutically acceptable carrier” refers to a non-toxic solid, semisolid or liquid filler, diluent, encapsulating material or formulation auxiliary of any. The term “parenteral” as used herein refers to modes of administration which include intravenous, intramuscular, intraperitoneal, intrasternal, subcutaneous and intraarticular injection and infusion.

Therapeutics of the invention are also suitably administered by sustained-release systems. Suitable examples of sustained-release Therapeutics are administered orally, rectally, parenterally, intracisternally, intravaginally, intraperitoneally, topically (as by powders, ointments, gels, drops or transdermal patch), bucally, or as an oral or nasal spray. “Pharmaceutically acceptable carrier” refers to a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion.

Therapeutics of the invention may also be suitably administered by sustained-release systems. Suitable examples of sustained-release Therapeutics include suitable polymeric materials (such as, for example, semi-permeable polymer matrices in the form of shaped articles, e.g., films, or microcapsules), suitable hydrophobic materials (for example as an emulsion in an acceptable oil) or ion exchange resins, and sparingly soluble derivatives (such as, for example, a sparingly soluble salt).

Sustained-release matrices include polylactides (U.S. Pat. No. 3,773,919, EP 58,481), copolymers of L-glutamic acid and gamma-ethyl-L-glutamate (Sidman et al., Biopolymers 22:547-556 (1983)), poly (2-hydroxyethyl methacrylate) (Langer et al., J. Biomed. Mater. Res. 15:167-277 (1981), and Langer, Chem. Tech. 12:98-105 (1982)), ethylene vinyl acetate (Langer et al., Id.) or poly-D-(−)-3-hydroxybutyric acid (EP 133,988).

Sustained-release Therapeutics also include liposomally entrapped Therapeutics of the invention (see, generally, Langer, Science 249:1527-1533 (1990); Treat et al., in Liposomes in the Therapy of Infectious Disease and Cancer, Lopez-Berestein and Fidler (eds.), Liss, N.Y., pp. 317 -327 and 353-365 (1989)). Liposomes containing the Therapeutic are prepared by methods known per se: DE 3,218,121, Epstein et al., Proc. Natl. Acad. Sci. (USA) 82:3688-3692 (1985); Hwang et al., Proc. Natl. Acad. Sci.(USA) 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal Therapeutic.

In yet an additional embodiment, the Therapeutics of the invention are delivered by way of a pump (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)).

Other controlled release systems are discussed in the review by Langer (Science 249:1527-1533 (1990)).

For parenteral administration, in one embodiment, the Therapeutic is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to the Therapeutic.

Generally, the formulations are prepared by contacting the Therapeutic uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium; and/or nonionic surfactants such as polysorbates, poloxamers, or PEG.

The Therapeutic will typically be formulated in such vehicles at a concentration of about 0.1 mg/ml to 100 mg/ml, preferably 1-10 mg/ml, at a pH of about 3 to 8. It will be understood that the use of certain of the foregoing excipients, carriers, or stabilizers will result in the formation of polypeptide salts.

Any pharmaceutical used for therapeutic administration can be sterile. Sterility is readily accomplished by filtration through sterile filtration membranes (e.g., 0.2 micron membranes). Therapeutics generally are placed into a container having a sterile access port, for example, an intravenous solution bag or vial having a stopper pierceable by a hypodermic injection needle.

Therapeutics ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous Therapeutic solution, and the resulting mixture is lyophilized. The infusion solution is prepared by reconstituting the lyophilized Therapeutic using bacteriostatic Water-for-Injection.

The invention also provides a pharmaceutical pack or kit comprising one or more containers filled with one or more of the ingredients of the Therapeutics of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, the Therapeutics may be employed in conjunction with other therapeutic compounds.

The Therapeutics of the invention may be administered alone or in combination with adjuvants. Adjuvants that may be administered with the Therapeutics of the invention include, but are not limited to, alum, alum plus deoxycholate (ImmunoAg), MTP-PE (Biocine Corp.), QS21 (Genentech, Inc.), BCG, and MPL. In a specific embodiment, Therapeutics of the invention are administered in combination with alum. In another specific embodiment, Therapeutics of the invention are administered in combination with QS-21. Further adjuvants that may be administered with the Therapeutics of the invention include, but are not limited to, Monophosphoryl lipid immunomodulator, AdjuVax 100a, QS-21, QS-18, CRL1005, Aluminum salts, MF-59, and Virosomal adjuvant technology. Vaccines that may be administered with the Therapeutics of the invention include, but are not limited to, vaccines directed toward protection against MMR (measles, mumps, rubella), polio, varicella, tetanus/diptheria, hepatitis A, hepatitis B, haemophilus influenzae B, whooping cough, pneumonia, influenza, Lyme's Disease, rotavirus, cholera, yellow fever, Japanese encephalitis, poliomyelitis, rabies, typhoid fever, and pertussis. Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

The Therapeutics of the invention may be administered alone or in combination with other therapeutic agents. Therapeutic agents that may be administered in combination with the Therapeutics of the invention, include but not limited to, other members of the TNF family, chemotherapeutic agents, antibiotics, steroidal and non-steroidal anti-inflammatories, conventional immunotherapeutic agents, cytokines and/or growth factors. Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

In one embodiment, the Therapeutics of the invention are administered in combination with members of the TNF family. TNF, TNF-related or TNF-like molecules that may be administered with the Therapeutics of the invention include, but are not limited to, soluble forms of TNF-alpha, lymphotoxin-alpha (LT-alpha, also known as TNF-beta), LT-beta (found in complex heterotrimer LT-alpha2-beta), OPGL, FasL, CD27L, CD30L, CD40L, 4-1BBL, DcR3, OX40L, TNF-gamma (International Publication No. WO 96/14328), AIM-I (International Publication No. WO 97/33899), endokine-alpha (International Publication No. WO 98/07880), TR6 (International Publication No. WO 98/30694), OPG, and neutrokine-alpha (International Publication No. WO 98/18921, OX40, and nerve growth factor (NGF), and soluble forms of Fas, CD30, CD27, CD40 and 4-IBB, TR2 (International Publication No. WO 96/34095), DR3 (International Publication No. WO 97/33904), DR4 (International Publication No. WO 98/32856), TR5 (International Publication No. WO 98/30693), TR6 (International Publication No. WO 98/30694), TR7 (International Publication No. WO 98/41629), TRANK, TR9 (International Publication No. WO 98/56892),TRIO (International Publication No. WO 98/54202), 312C2 (International Publication No. WO 98/06842), and TR12, and soluble forms CD154, CD70, and CD153.

In certain embodiments, Therapeutics of the invention are administered in combination with antiretroviral agents, nucleoside reverse transcriptase inhibitors, non-nucleoside reverse transcriptase inhibitors, and/or protease inhibitors. Nucleoside reverse transcriptase inhibitors that may be administered in combination with the Therapeutics of the invention, include, but are not limited to, RETROVIR (zidovudine/AZT), VIDEX (didanosine/ddl), HIVID (zalcitabine/ddC), ZERIT (stavudine/d4T), EPIVIR (lamivudine/3TC), and COMBIVIR (zidovudine/lamivudine). Non-nucleoside reverse transcriptase inhibitors that may be administered in combination with the Therapeutics of the invention, include, but are not limited to, VIRAMUNE (nevirapine), RESCRIPTOR (delavirdine), and SUSTIVA (efavirenz). Protease inhibitors that may be administered in combination with the Therapeutics of the invention, include, but are not limited to, CRIXIVAN (indinavir), NORVIR (ritonavir), INVIRASE (saquinavir), and VIRACEPT (nelfinavir). In a specific embodiment, antiretroviral agents, nucleoside reverse transcriptase inhibitors, non-nucleoside reverse transcriptase inhibitors, and/or protease inhibitors may be used in any combination with Therapeutics of the invention to treat AIDS and/or to prevent or treat HIV infection.

In other embodiments, Therapeutics of the invention may be administered in combination with anti-opportunistic infection agents. Anti-opportunistic agents that may be administered in combination with the Therapeutics of the invention, include, but are not limited to, TRIMETHOPRIM-SULFAMETHOXAZOLE, DAPSONE, PENTAMIDINE, ATOVAQUONE, ISONIAZID, RIFAMPIN, PYRAZINAMIDE, ETHAMBUTOL, RIFABUTIN, CLARITHROMYCIN, AZITHROMYCIN, GANCICLOVIR, FOSCARNET, CIDOFOVIR, FLUCONAZOLE, ITRACONAZOLE, KETOCONAZOLE, ACYCLOVIR, FAMCICOLVIR, PYRIMETHAMINE, LEUCOVORIN, NEUPOGEN (filgrastim/G-CSF), and LEUKINE (sargramostim/GM-CSF). In a specific embodiment, Therapeutics of the invention are used in any combination with TRIMETHOPRIM-SULFAMETHOXAZOLE, DAPSONE, PENTAMIDINE, and/or ATOVAQUONE to prophylactically treat or prevent an opportunistic Pneumocystis carinii pneumonia infection. In another specific embodiment, Therapeutics of the invention are used in any combination with ISONIAZID, RIFAMPIN, PYRAZINAMIDE, and/or ETHAMBUTOL to prophylactically treat or prevent an opportunistic Mycobacterium avium complex infection. In another specific embodiment, Therapeutics of the invention are used in any combination with RIFABUTIN, CLARITHROMYCIN, and/or AZITHROMYCIN to prophylactically treat or prevent an opportunistic Mycobacterium tuberculosis infection. In another specific embodiment, Therapeutics of the invention are used in any combination with GANCICLOVIR, FOSCARNET, and/or CIDOFOVIR to prophylactically treat or prevent an opportunistic cytomegalovirus infection. In another specific embodiment, Therapeutics of the invention are used in any combination with FLUCONAZOLE, ITRACONAZOLE, and/or KETOCONAZOLE to prophylactically treat or prevent an opportunistic fungal infection. In another specific embodiment, Therapeutics of the invention are used in any combination with ACYCLOVIR and/or FAMCICOLVIR to prophylactically treat or prevent an opportunistic herpes simplex virus type I and/or type II infection. In another specific embodiment, Therapeutics of the invention are used in any combination with PYRIMETHAMINE and/or LEUCOVORIN to prophylactically treat or prevent an opportunistic Toxoplasma gondii infection. In another specific embodiment, Therapeutics of the invention are used in any combination with LEUCOVORIN and/or NEUPOGEN to prophylactically treat or prevent an opportunistic bacterial infection.

In a further embodiment, the Therapeutics of the invention are administered in combination with an antiviral agent. Antiviral agents that may be administered with the Therapeutics of the invention include, but are not limited to, acyclovir, ribavirin, amantadine, and remantidine.

In a further embodiment, the Therapeutics of the invention are administered in combination with an antibiotic agent. Antibiotic agents that may be administered with the Therapeutics of the invention include, but are not limited to, amoxicillin, beta-lactamases, aminoglycosides, beta-lactam (glycopeptide), beta-lactamases, Clindamycin, chloramphenicol, cephalosporins, ciprofloxacin, ciprofloxacin, erythromycin, fluoroquinolones, macrolides, metronidazole, penicillins, quinolones, rifampin, streptomycin, sulfonamide, tetracyclines, trimethoprim, trimethoprim-sulfamthoxazole, and vancomycin.

Conventional nonspecific immunosuppressive agents, that may be administered in combination with the Therapeutics of the invention include, but are not limited to, steroids, cyclosporine, cyclosporine analogs, cyclophosphamide methylprednisone, prednisone, azathioprine, FK-506, 15-deoxyspergualin, and other immunosuppressive agents that act by suppressing the function of responding T cells.

In specific embodiments, Therapeutics of the invention are administered in combination with immunosuppressants. Immunosuppressants preparations that may be administered with the Therapeutics of the invention include, but are not limited to, ORTHOCLONE (OKT3), SANDIMMUNE/NEORAL/SANGDYA (cyclosporin), PROGRAF (tacrolimus), CELLCEPT (mycophenolate), Azathioprine, glucorticosteroids, and RAPAMUNE (sirolimus). In a specific embodiment, immunosuppressants may be used to prevent rejection of organ or bone marrow transplantation.

In an additional embodiment, Therapeutics of the invention are administered alone or in combination with one or more intravenous immune globulin preparations. Intravenous immune globulin preparations that may be administered with the Therapeutics of the invention include, but not limited to, GAMMAR, IVEEGAM, SANDOGLOBULIN, GAMMAGARD S/D, and GAMIMUNE. In a specific embodiment, Therapeutics of the invention are administered in combination with intravenous immune globulin preparations in transplantation therapy (e.g., bone marrow transplant).

In an additional embodiment, the Therapeutics of the invention are administered alone or in combination with an anti-inflammatory agent. Anti-inflammatory agents that may be administered with the Therapeutics of the invention include, but are not limited to, glucocorticoids and the nonsteroidal anti-inflammatories, aminoarylcarboxylic acid derivatives, arylacetic acid derivatives, arylbutyric acid derivatives, arylcarboxylic acids, arylpropionic acid derivatives, pyrazoles, pyrazolones, salicylic acid derivatives, thiazinecarboxamides, e-acetamidocaproic acid, S-adenosylmethionine, 3-amino-4-hydroxybutyric acid, amixetrine, bendazac, benzydamine, bucolome, difenpiramide, ditazol, emorfazone, guaiazulene, nabumetone, nimesulide, orgotein, oxaceprol, paranyline, perisoxal, pifoxime, proquazone, proxazole, and tenidap.

In another embodiment, compositions of the invention are administered in combination with a chemotherapeutic agent. Chemotherapeutic agents that may be administered with the Therapeutics of the invention include, but are not limited to, antibiotic derivatives (e.g., doxorubicin, bleomycin, daunorubicin, and dactinomycin); antiestrogens (e.g., tamoxifen); antimetabolites (e.g., fluorouracil, 5-FU, methotrexate, floxuridine, interferon alpha-2b, glutamic acid, plicamycin, mercaptopurine, and 6-thioguanine); cytotoxic agents (e.g., carmustine, BCNU, lomustine, CCNU, cytosine arabinoside, cyclophosphamide, estramustine, hydroxyurea, procarbazine, mitomycin, busulfan, cis-platin, and vincristine sulfate); hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone); nitrogen mustard derivatives (e.g., mephalen, chorambucil, mechlorethamine (nitrogen mustard) and thiotepa); steroids and combinations (e.g., bethamethasone sodium phosphate); and others (e.g., dicarbazine, asparaginase, mitotane, vincristine sulfate, vinblastine sulfate, and etoposide).

In a specific embodiment, Therapeutics of the invention are administered in combination with CHOP (cyclophosphamide, doxorubicin, vincristine, and prednisone) or any combination of the components of CHOP. In another embodiment, Therapeutics of the invention are administered in combination with Rituximab. In a further embodiment, Therapeutics of the invention are administered with Rituxmab and CHOP, or Rituxmab and any combination of the components of CHOP.

In an additional embodiment, the Therapeutics of the invention are administered in combination with cytokines. Cytokines that may be administered with the Therapeutics of the invention include, but are not limited to, IL2, IL3, IL4, IL5, IL6, IL7, IL10, IL-12, IL-13, IL-15, anti-CD40, CD40L, IFN-gamma and TNF-alpha. In another embodiment, Therapeutics of the invention may be administered with any interleukin, including, but not limited to, IL-1alpha, IL-1beta, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, and IL-21.

In an additional embodiment, the Therapeutics of the invention are administered in combination with angiogenic proteins. Angiogenic proteins that may be administered with the Therapeutics of the invention include, but are not limited to, Glioma Derived Growth Factor (GDGF), as disclosed in European Patent Number EP-399816; Platelet Derived Growth Factor-A (PDGF-A), as disclosed in European Patent Number EP-6821 10; Platelet Derived Growth Factor-B (PDGF-B), as disclosed in European Patent Number EP-282317; Placental Growth Factor (PlGF), as disclosed in International Publication Number WO 92/06194; Placental Growth Factor-2 (PIGF-2), as disclosed in Hauser et al., Gorwth Factors, 4:259-268 (1993); Vascular Endothelial Growth Factor (VEGF), as disclosed in International Publication Number WO 90/13649; Vascular Endothelial Growth Factor-A (VEGF-A), as disclosed in European Patent Number EP-506477; Vascular Endothelial Growth Factor-2 (VEGF-2), as disclosed in International Publication Number WO 96/39515; Vascular Endothelial Growth Factor B (VEGF-3); Vascular Endothelial Growth Factor B-186 (VEGF-B186), as disclosed in International Publication Number WO 96/26736; Vascular Endothelial Growth Factor-D (VEGF-D), as disclosed in International Publication Number WO 98/02543; Vascular Endothelial Growth Factor-D (VEGF-D), as disclosed in International Publication Number WO 98/07832; and Vascular Endothelial Growth Factor-E (VEGF-E), as disclosed in German Patent Number DE19639601. The above mentioned references are incorporated herein by reference herein.

In an additional embodiment, the Therapeutics of the invention are administered in combination with hematopoietic growth factors. Hematopoietic growth factors that may be administered with the Therapeutics of the invention include, but are not limited to, LEUKINE (SARGRAMOSTIM) and NEUPOGEN (FILGRASTIM).

In an additional embodiment, the Therapeutics of the invention are administered in combination with Fibroblast Growth Factors. Fibroblast Growth Factors that may be administered with the Therapeutics of the invention include, but are not limited to, FGF-1, FGF-2, FGF-3, FGF-4, FGF-5, FGF-6, FGF-7, FGF-8, FGF-9, FGF-10, FGF-11, FGF-12, FGF-13, FGF-14, and FGF-15.

In a specific embodiment, formulations of the present invention may further comprise antagonists of P-glycoprotein (also referred to as the multiresistance protein, or PGP), including antagonists of its encoding polynucleotides (e.g., antisense oligonucleotides, ribozymes, zinc-finger proteins, etc.). P-glycoprotein is well known for decreasing the efficacy of various drug administrations due to its ability to export intracellular levels of absorbed drug to the cell exterior. While this activity has been particularly pronounced in cancer cells in response to the administration of chemotherapy regimens, a variety of other cell types and the administration of other drug classes have been noted (e.g., T-cells and anti-HIV drugs). In fact, certain mutations in the PGP gene significantly reduces PGP function, making it less able to force drugs out of cells. People who have two versions of the mutated gene—one inherited from each parent—have more than four times less PGP than those with two normal versions of the gene. People may also have one normal gene and one mutated one. Certain ethnic populations have increased incidence of such PGP mutations. Among individuals from Ghana, Kenya, the Sudan, as well as African Americans, frequency of the normal gene ranged from 73% to 84%. In contrast, the frequency was 34% to 59% among British whites, Portuguese, Southwest Asian, Chinese, Filipino and Saudi populations. As a result, certain ethnic populations may require increased administration of PGP antagonist in the formulation of the present invention to arrive at the an efficacious dose of the therapeutic (e.g., those from African descent). Conversely, certain ethnic populations, particularly those having increased frequency of the mutated PGP (e.g., of Caucasian descent, or non-African descent) may require less pharmaceutical compositions in the formulation due to an effective increase in efficacy of such compositions as a result of the increased effective absorption (e.g., less PGP activity) of said composition.

Moreover, in another specific embodiment, formulations of the present invention may further comprise antagonists of OATP2 (also referred to as the multiresistance protein, or MRP2), including antagonists of its encoding polynucleotides (e.g., antisense oligonucleotides, ribozymes, zinc-finger proteins, etc.). The invention also further comprises any additional antagonists known to inhibit proteins thought to be attributable to a multidrug resistant phenotype in proliferating cells.

Preferred antagonists that formulations of the present may comprise include the potent P-glycoprotein inhibitor elacridar, and/or LY-335979. Other P-glycoprotein inhibitors known in the art are also encompassed by the present invention.

In additional embodiments, the Therapeutics of the invention are administered in combination with other therapeutic or prophylactic regimens, such as, for example, radiation therapy.

### Example 22

### Method of Treating Decreased Levels of the Polypeptide

The present invention relates to a method for treating an individual in need of an increased level of a polypeptide of the invention in the body comprising administering to such an individual a composition comprising a therapeutically effective amount of an agonist of the invention (including polypeptides of the invention). Moreover, it will be appreciated that conditions caused by a decrease in the standard or normal expression level of a secreted protein in an individual can be treated by administering the polypeptide of the present invention, preferably in the secreted form. Thus, the invention also provides a method of treatment of an individual in need of an increased level of the polypeptide comprising administering to such an individual a Therapeutic comprising an amount of the polypeptide to increase the activity level of the polypeptide in such an individual.

For example, a patient with decreased levels of a polypeptide receives a daily dose 0.1-100 ug/kg of the polypeptide for six consecutive days. Preferably, the polypeptide is in the secreted form. The exact details of the dosing scheme, based on administration and formulation, are provided herein.

### Example 23

### Method of Treating Increased Levels of the Polypeptide

The present invention also relates to a method of treating an individual in need of a decreased level of a polypeptide of the invention in the body comprising administering to such an individual a composition comprising a therapeutically effective amount of an antagonist of the invention (including polypeptides and antibodies of the invention).

In one example, antisense technology is used to inhibit production of a polypeptide of the present invention. This technology is one example of a method of decreasing levels of a polypeptide, preferably a secreted form, due to a variety of etiologies, such as cancer. For example, a patient diagnosed with abnormally increased levels of a polypeptide is administered intravenously antisense polynucleotides at 0.5, 1.0, 1.5, 2.0 and 3.0 mg/kg day for 21 days. This treatment is repeated after a 7-day rest period if the treatment was well tolerated. The formulation of the antisense polynucleotide is provided herein.

### Example 24

### Method of Treatment Using Gene Therapy-Ex Vivo

One method of gene therapy transplants fibroblasts, which are capable of expressing a polypeptide, onto a patient. Generally, fibroblasts are obtained from a subject by skin biopsy. The resulting tissue is placed in tissue-culture medium and separated into small pieces. Small chunks of the tissue are placed on a wet surface of a tissue culture flask, approximately ten pieces are placed in each flask. The flask is turned upside down, closed tight and left at room temperature over night. After 24 hours at room temperature, the flask is inverted and the chunks of tissue remain fixed to the bottom of the flask and fresh media (e.g., Ham's F12 media, with 10% FBS, penicillin and streptomycin) is added. The flasks are then incubated at 37 degree C for approximately one week.

At this time, fresh media is added and subsequently changed every several days. After an additional two weeks in culture, a monolayer of fibroblasts emerge. The monolayer is trypsinized and scaled into larger flasks.

pMV-7 (Kirschmeier, P. T. et al., DNA, 7:219-25 (1988)), flanked by the long terminal repeats of the Moloney murine sarcoma virus, is digested with EcoRI and HindIII and subsequently treated with calf intestinal phosphatase. The linear vector is fractionated on agarose gel and purified, using glass beads.

The cDNA encoding a polypeptide of the present invention can be amplified using PCR primers which correspond to the 5′ and 3′ end sequences respectively as set forth in the Examples herein or otherwise known in the art, using primers and having appropriate restriction sites and initiation/stop codons, if necessary. Preferably, the 5′ primer contains an EcoRI site and the 3′ primer includes a HindIII site. Equal quantities of the Moloney murine sarcoma virus linear backbone and the amplified EcoRI and HindIII fragment are added together, in the presence of T4 DNA ligase. The resulting mixture is maintained under conditions appropriate for ligation of the two fragments. The ligation mixture is then used to transform bacteria HB101, which are then plated onto agar containing kanamycin for the purpose of confirming that the vector has the gene of interest properly inserted.

The amphotropic pA317 or GP+am12 packaging cells are grown in tissue culture to confluent density in Dulbecco's Modified Eagles Medium (DMEM) with 10% calf serum (CS), penicillin and streptomycin. The MSV vector containing the gene is then added to the media and the packaging cells transduced with the vector. The packaging cells now produce infectious viral particles containing the gene (the packaging cells are now referred to as producer cells).

Fresh media is added to the transduced producer cells, and subsequently, the media is harvested from a 10 cm plate of confluent producer cells. The spent media, containing the infectious viral particles, is filtered through a millipore filter to remove detached producer cells and this media is then used to infect fibroblast cells. Media is removed from a sub-confluent plate of fibroblasts and quickly replaced with the media from the producer cells. This media is removed and replaced with fresh media. If the titer of virus is high, then virtually all fibroblasts will be infected and no selection is required. If the titer is very low, then it is necessary to use a retroviral vector that has a selectable marker, such as neo or his. Once the fibroblasts have been efficiently infected, the fibroblasts are analyzed to determine whether protein is produced.

The engineered fibroblasts are then transplanted onto the host, either alone or after having been grown to confluence on cytodex 3 microcarrier beads.

### Example 25

### Gene Therapy Using Endogenous Genes Corresponding to Polynucleotides of the Invention

Another method of gene therapy according to the present invention involves operably associating the endogenous polynucleotide sequence of the invention with a promoter via homologous recombination as described, for example, in U.S. Pat. No. 5,641,670, issued Jun. 24, 1997; International Publication NO: WO 96/29411, published Sep. 26, 1996; International Publication NO: WO 94/12650, published Aug. 4, 1994; Koller et al., Proc. Natl. Acad. Sci. USA, 86:8932-8935 (1989); and Zijlstra et al., Nature, 342:435-438 (1989). This method involves the activation of a gene which is present in the target cells, but which is not expressed in the cells, or is expressed at a lower level than desired.

Polynucleotide constructs are made which contain a promoter and targeting sequences, which are homologous to the 5′ non-coding sequence of endogenous polynucleotide sequence, flanking the promoter. The targeting sequence will be sufficiently near the 5′ end of the polynucleotide sequence so the promoter will be operably linked to the endogenous sequence upon homologous recombination. The promoter and the targeting sequences can be amplified using PCR. Preferably, the amplified promoter contains distinct restriction enzyme sites on the 5′ and 3′ ends. Preferably, the 3′ end of the first targeting sequence contains the same restriction enzyme site as the 5′ end of the amplified promoter and the 5′ end of the second targeting sequence contains the same restriction site as the 3′ end of the amplified promoter.

The amplified promoter and the amplified targeting sequences are digested with the appropriate restriction enzymes and subsequently treated with calf intestinal phosphatase. The digested promoter and digested targeting sequences are added together in the presence of T4 DNA ligase. The resulting mixture is maintained under conditions appropriate for ligation of the two fragments. The construct is size fractionated on an agarose gel then purified by phenol extraction and ethanol precipitation.

In this Example, the polynucleotide constructs are administered as naked polynucleotides via electroporation. However, the polynucleotide constructs may also be administered with transfection-facilitating agents, such as liposomes, viral sequences, viral particles, precipitating agents, etc. Such methods of delivery are known in the art.

Once the cells are transfected, homologous recombination will take place which results in the promoter being operably linked to the endogenous polynucleotide sequence. This results in the expression of polynucleotide corresponding to the polynucleotide in the cell. Expression may be detected by immunological staining, or any other method known in the art.

Fibroblasts are obtained from a subject by skin biopsy. The resulting tissue is placed in DMEM+10% fetal calf serum. Exponentially growing or early stationary phase fibroblasts are trypsinized and rinsed from the plastic surface with nutrient medium. An aliquot of the cell suspension is removed for counting, and the remaining cells are subjected to centrifugation. The supernatant is aspirated and the pellet is resuspended in 5 ml of electroporation buffer (20 mM HEPES pH 7.3, 137 mM NaCl, 5 mM KCl, 0.7 mM Na2 HPO4, 6 mM dextrose). The cells are recentrifuged, the supernatant aspirated, and the cells resuspended in electroporation buffer containing 1 mg/ml acetylated bovine serum albumin. The final cell suspension contains approximately 3×106 cells/ml. Electroporation should be performed immediately following resuspension.

Plasmid DNA is prepared according to standard techniques. For example, to construct a plasmid for targeting to the locus corresponding to the polynucleotide of the invention, plasmid pUC18 (MBI Fermentas, Amherst, N.Y.) is digested with HindIII. The CMV promoter is amplified by PCR with an XbaI site on the 5′ end and a BamHI site on the 3′end. Two non-coding sequences are amplified via PCR: one non-coding sequence (fragment 1) is amplified with a HindIII site at the 5′ end and an Xba site at the 3′end; the other non-coding sequence (fragment 2) is amplified with a BamHI site at the 5′end and a HindIII site at the 3′end. The CMV promoter and the fragments (1 and 2) are digested with the appropriate enzymes (CMV promoter—XbaI and BamHI; fragment 1-XbaI; fragment 2-BamHI) and ligated together. The resulting ligation product is digested with HindIII, and ligated with the HindIII-digested pUC18 plasmid.

Plasmid DNA is added to a sterile cuvette with a 0.4 cm electrode gap (Bio-Rad). The final DNA concentration is generally at least 120 μg/ml. 0.5 ml of the cell suspension (containing approximately 1.5.×106 cells) is then added to the cuvette, and the cell suspension and DNA solutions are gently mixed. Electroporation is performed with a Gene-Pulser apparatus (Bio-Rad). Capacitance and voltage are set at 960 μF and 250-300 V, respectively. As voltage increases, cell survival decreases, but the percentage of surviving cells that stably incorporate the introduced DNA into their genome increases dramatically. Given these parameters, a pulse time of approximately 14-20 mSec should be observed.

Electroporated cells are maintained at room temperature for approximately 5 min, and the contents of the cuvette are then gently removed with a sterile transfer pipette. The cells are added directly to 10 ml of prewarmed nutrient media (DMEM with 15% calf serum) in a 10 cm dish and incubated at 37 degree C. The following day, the media is aspirated and replaced with 10 ml of fresh media and incubated for a further 16-24 hours.

The engineered fibroblasts are then injected into the host, either alone or after having been grown to confluence on cytodex 3 microcarrier beads. The fibroblasts now produce the protein product. The fibroblasts can then be introduced into a patient as described above.

### Example 26

### Method of Treatment Using Gene Therapy—In Vivo

Another aspect of the present invention is using in vivo gene therapy methods to treat disorders, diseases and-conditions. The gene therapy method relates to the introduction of naked nucleic acid (DNA, RNA, and antisense DNA or RNA) sequences into an animal to increase or decrease the expression of the polypeptide. The polynucleotide of the present invention may be operatively linked to a promoter or any other genetic elements necessary for the expression of the polypeptide by the target tissue. Such gene therapy and delivery techniques and methods are known in the art, see, for example, WO90/11092, WO98/11779; U.S. Pat. Nos. 5,693,622, 5,705,151, 5,580,859; Tabata et al., Cardiovasc. Res. 35(3):470-479 (1997); Chao et al., Pharmacol. Res. 35(6):517-522 (1997); Wolff, Neuromuscul. Disord. 7(5):314-318 (1997); Schwartz et al., Gene Ther. 3(5):405-411 (1996); Tsurumi et al., Circulation 94(12):3281-3290 (1996) (incorporated herein by reference).

The polynucleotide constructs may be delivered by any method that delivers injectable materials to the cells of an animal, such as, injection into the interstitial space of tissues (heart, muscle, skin, lung, liver, intestine and the like). The polynucleotide constructs can be delivered in a pharmaceutically acceptable liquid or aqueous carrier.

The term “naked” polynucleotide, DNA or RNA, refers to sequences that are free from any delivery vehicle that acts to assist, promote, or facilitate entry into the cell, including viral sequences, viral particles, liposome formulations, lipofectin or precipitating agents and the like. However, the polynucleotides of the present invention may also be delivered in liposome formulations (such as those taught in Felgner P. L. et al. (1995) Ann. NY Acad. Sci. 772:126-139 and Abdallah B. et al. (1995) Biol. Cell 85(1):1-7) which can be prepared by methods well known to those skilled in the art.

The polynucleotide vector constructs used in the gene therapy method are preferably constructs that will not integrate into the host genome nor will they contain sequences that allow for replication. Any strong promoter known to those skilled in the art can be used for driving the expression of DNA. Unlike other gene therapies techniques, one major advantage of introducing naked nucleic acid sequences into target cells is the transitory nature of the polynucleotide synthesis in the cells. Studies have shown that non-replicating DNA sequences can be introduced into cells to provide production of the desired polypeptide for periods of up to six months.

The polynucleotide construct can be delivered to the interstitial space of tissues within the an animal, including of muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue. Interstitial space of the tissues comprises the intercellular fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels. Delivery to the interstitial space of muscle tissue is preferred for the reasons discussed below. They may be conveniently delivered by injection into the tissues comprising these cells. They are preferably delivered to and expressed in persistent, non-dividing cells which are differentiated, although delivery and expression may be achieved in non-differentiated or less completely differentiated cells, such as, for example, stem cells of blood or skin fibroblasts. In vivo muscle cells are particularly competent in their ability to take up and express polynucleotides.

For the naked polynucleotide injection, an effective dosage amount of DNA or RNA will be in the range of from about 0.05 g/kg body weight to about 50 mg/kg body weight. Preferably the dosage will be from about 0.005 mg/kg to about 20 mg/kg and more preferably from about 0.05 mg/kg to about 5 mg/kg. Of course, as the artisan of ordinary skill will appreciate, this dosage will vary according to the tissue site of injection. The appropriate and effective dosage of nucleic acid sequence can readily be determined by those of ordinary skill in the art and may depend on the condition being treated and the route of administration. The preferred route of administration is by the parenteral route of injection into the interstitial space of tissues. However, other parenteral routes may also be used, such as, inhalation of an aerosol formulation particularly for delivery to lungs or bronchial tissues, throat or mucous membranes of the nose. In addition, naked polynucleotide constructs can be delivered to arteries during angioplasty by the catheter used in the procedure.

The dose response effects of injected polynucleotide in muscle in vivo is determined as follows. Suitable template DNA for production of mRNA coding for polypeptide of the present invention is prepared in accordance with a standard recombinant DNA methodology. The template DNA, which may be either circular or linear, is either used as naked DNA or complexed with liposomes. The quadriceps muscles of mice are then injected with various amounts of the template DNA.

Five to six week old female and male Balb/C mice are anesthetized by intraperitoneal injection with 0.3 ml of 2.5% Avertin. A 1.5 cm incision is made on the anterior thigh, and the quadriceps muscle is directly visualized. The template DNA is injected in 0.1 ml of carrier in a 1 cc syringe through a 27 gauge needle over one minute, approximately 0.5 cm from the distal insertion site of the muscle into the knee and about 0.2 cm deep. A suture is placed over the injection site for future localization, and the skin is closed with stainless steel clips.

After an appropriate incubation time (e.g., 7 days) muscle extracts are prepared by excising the entire quadriceps. Every fifth 15 um cross-section of the individual quadriceps muscles is histochemically stained for protein expression. A time course for protein expression may be done in a similar fashion except that quadriceps from different mice are harvested at different times. Persistence of DNA in muscle following injection may be determined by Southern blot analysis after preparing total cellular DNA and HIRT supernatants from injected and control mice. The results of the above experimentation in mice can be use to extrapolate proper dosages and other treatment parameters in humans and other animals using naked DNA.

### Example 27

### Transgenic Animals

The polypeptides of the invention can also be expressed in transgenic animals. Animals of any species, including, but not limited to, mice, rats, rabbits, hamsters, guinea pigs, pigs, micro-pigs, goats, sheep, cows and non-human primates, e.g., baboons, monkeys, and chimpanzees may be used to generate transgenic animals. In a specific embodiment, techniques described herein or otherwise known in the art, are used to express polypeptides of the invention in humans, as part of a gene therapy protocol.

Any technique known in the art may be used to introduce the transgene (i.e., polynucleotides of the invention) into animals to produce the founder lines of transgenic animals. Such techniques include, but are not limited to, pronuclear microinjection (Paterson et al., Appl. Microbiol. Biotechnol. 40:691-698 (1994); Carver et al., Biotechnology (NY) 11:1263-1270 (1993); Wright et al., Biotechnology (NY) 9:830-834 (1991); and Hoppe et al., U.S. Pat. No. 4,873,191 (1989)); retrovirus mediated gene transfer into germ lines (Van der Putten et al., Proc. Natl. Acad. Sci., USA 82:6148-6152 (1985)), blastocysts or embryos; gene targeting in embryonic stem cells (Thompson et al., Cell 56:313-321 (1989)); electroporation of cells or embryos (Lo, 1983, Mol Cell. Biol. 3:1803-1814 (1983)); introduction of the polynucleotides of the invention using a gene gun (see, e.g., Ulmer et al., Science 259:1745 (1993); introducing nucleic acid constructs into embryonic pleuripotent stem cells and transferring the stem cells back into the blastocyst; and sperm-mediated gene transfer (Lavitrano et al., Cell 57:717-723 (1989); etc. For a review of such techniques, see Gordon, “Transgenic Animals” Intl. Rev. Cytol. 115:171-229 (1989), which is incorporated by reference herein in its entirety.

Any technique known in the art may be used to produce transgenic clones containing polynucleotides of the invention, for example, nuclear transfer into enucleated oocytes of nuclei from cultured embryonic, fetal, or adult cells induced to quiescence (Campell et al., Nature 380:64-66 (1996); Wilmut et al., Nature 385:810-813 (1997)).

The present invention provides for transgenic animals that carry the transgene in all their cells, as well as animals which carry the transgene in some, but not all their cells, i.e., mosaic animals or chimeric. The transgene may be integrated as a single transgene or as multiple copies such as in concatamers, e.g., head-to-head tandems or head-to-tail tandems. The transgene may also be selectively introduced into and activated in a particular cell type by following, for example, the teaching of Lasko et al. (Lasko et al., Proc. Natl. Acad. Sci. USA 89:6232-6236 (1992)). The regulatory sequences required for such a cell-type specific activation will depend upon the particular cell type of interest, and will be apparent to those of skill in the art. When it is desired that the polynucleotide transgene be integrated into the chromosomal site of the endogenous gene, gene targeting is preferred. Briefly, when such a technique is to be utilized, vectors containing some nucleotide sequences homologous to the endogenous gene are designed for the purpose of integrating, via homologous recombination with chromosomal sequences, into and disrupting the function of the nucleotide sequence of the endogenous gene. The transgene may also be selectively introduced into a particular cell type, thus inactivating the endogenous gene in only that cell type, by following, for example, the teaching of Gu et al. (Gu et al., Science 265:103-106 (1994)). The regulatory sequences required for such a cell-type specific inactivation will depend upon the particular cell type of interest, and will be apparent to those of skill in the art.

Once transgenic animals have been generated, the expression of the recombinant gene may be assayed utilizing standard techniques. Initial screening may be accomplished by Southern blot analysis or PCR techniques to analyze animal tissues to verify that integration of the transgene has taken place. The level of mRNA expression of the transgene in the tissues of the transgenic animals may also be assessed using techniques which include, but are not limited to, Northern blot analysis of tissue samples obtained from the animal, in situ hybridization analysis, and reverse transcriptase-PCR(RT-PCR). Samples of transgenic gene-expressing tissue may also be evaluated immunocytochemically or immunohistochemically using antibodies specific for the transgene product.

Once the founder animals are produced, they may be bred, inbred, outbred, or crossbred to produce colonies of the particular animal. Examples of such breeding strategies include, but are not limited to: outbreeding of founder animals with more than one integration site in order to establish separate lines; inbreeding of separate lines in order to produce compound transgenics that express the transgene at higher levels because of the effects of additive expression of each transgene; crossing of heterozygous transgenic animals to produce animals homozygous for a given integration site in order to both augment expression and eliminate the need for screening of animals by DNA analysis; crossing of separate homozygous lines to produce compound heterozygous or homozygous lines; and breeding to place the transgene on a distinct background that is appropriate for an experimental model of interest.

Transgenic animals of the invention have uses which include, but are not limited to, animal model systems useful in elaborating the biological function of polypeptides of the present invention, studying diseases, disorders, and/or conditions associated with aberrant expression, and in screening for compounds effective in ameliorating such diseases, disorders, and/or conditions.

### Example 28

### Knock-Out Animals

Endogenous gene expression can also be reduced by inactivating or “knocking out” the gene and/or its promoter using targeted homologous recombination. (E.g., see Smithies et al., Nature 317:230-234 (1985); Thomas & Capecchi, Cell 51:503-512 (1987); Thompson et al., Cell 5:313-321 (1989); each of which is incorporated by reference herein in its entirety). For example, a mutant, non-functional polynucleotide of the invention (or a completely unrelated DNA sequence) flanked by DNA homologous to the endogenous polynucleotide sequence (either the coding regions or regulatory regions of the gene) can be used, with or without a selectable marker and/or a negative selectable marker, to transfect cells that express polypeptides of the invention in vivo. In another embodiment, techniques known in the art are used to generate knockouts in cells that contain, but do not express the gene of interest. Insertion of the DNA construct, via targeted homologous recombination, results in inactivation of the targeted gene. Such approaches are particularly suited in research and agricultural fields where modifications to embryonic stem cells can be used to generate animal offspring with an inactive targeted gene (e.g., see Thomas & Capecchi 1987 and Thompson 1989, supra). However this approach can be routinely adapted for use in humans provided the recombinant DNA constructs are directly administered or targeted to the required site in vivo using appropriate viral vectors that will be apparent to those of skill in the art.

In further embodiments of the invention, cells that are genetically engineered to express the polypeptides of the invention, or alternatively, that are genetically engineered not to express the polypeptides of the invention (e.g., knockouts) are administered to a patient in vivo. Such cells may be obtained from the patient (i.e., animal, including human) or an MHC compatible donor and can include, but are not limited to fibroblasts, bone marrow cells, blood cells (e.g., lymphocytes), adipocytes, muscle cells, endothelial cells etc. The cells are genetically engineered in vitro using recombinant DNA techniques to introduce the coding sequence of polypeptides of the invention into the cells, or alternatively, to disrupt the coding sequence and/or endogenous regulatory sequence associated with the polypeptides of the invention, e.g., by transduction (using viral vectors, and preferably vectors that integrate the transgene into the cell genome) or transfection procedures, including, but not limited to, the use of plasmids, cosmids, YACs, naked DNA, electroporation, liposomes, etc. The coding sequence of the polypeptides of the invention can be placed under the control of a strong constitutive or inducible promoter or promoter/enhancer to achieve expression, and preferably secretion, of the polypeptides of the invention. The engineered cells which express and preferably secrete the polypeptides of the invention can be introduced into the patient systemically, e.g., in the circulation, or intraperitoneally.

Alternatively, the cells can be incorporated into a matrix and implanted in the body, e.g., genetically engineered fibroblasts can be implanted as part of a skin graft; genetically engineered endothelial cells can be implanted as part of a lymphatic or vascular graft. (See, for example, Anderson et al. U.S. Pat. No. 5,399,349; and Mulligan & Wilson, U.S. Pat. No. 5,460,959 each of which is incorporated by reference herein in its entirety).

When the cells to be administered are non-autologous or non-MHC compatible cells, they can be administered using well known techniques which prevent the development of a host immune response against the introduced cells. For example, the cells may be introduced in an encapsulated form which, while allowing for an exchange of components with the immediate extracellular environment, does not allow the introduced cells to be recognized by the host immune system.

Transgenic and “knock-out” animals of the invention have uses which include, but are not limited to, animal model systems useful in elaborating the biological function of polypeptides of the present invention, studying diseases, disorders, and/or conditions associated with aberrant expression, and in screening for compounds effective in ameliorating such diseases, disorders, and/or conditions.

### Example 29

### Method of Creating N- and C-terminal Deletion Mutants Corresponding to the OATP2 and cMOAT Polypeptides of the Present Invention

As described elsewhere herein, the present invention encompasses the creation of N- and C-terminal deletion mutants, in addition to any combination of N- and C-terminal deletions thereof, corresponding to the OATP2 and cMOAT polypeptides of the present invention. A number of methods are available to one skilled in the art for creating such mutants. Such methods may include a combination of PCR amplification and gene cloning methodology. Although one of skill in the art of molecular biology, through the use of the teachings provided or referenced herein, and/or otherwise known in the art as standard methods, could readily create each deletion mutant of the present invention, exemplary methods are described below.

Briefly, using the isolated cDNA clone encoding the full-length OATP2 (SNP ID:PS100s2) polypeptide sequence (as described in Example 4 and 5, for example), appropriate primers of about 15-25 nucleotides derived from the desired 5′ and 3′ positions of SEQ ID NO:7 may be designed to PCR amplify, and subsequently clone, the intended N- and/or C-terminal deletion mutant. Such primers could comprise, for example, an inititation and stop codon for the 5′ and 3′ primer, respectively. Such primers may also comprise restriction sites to facilitate cloning of the deletion mutant post amplification. Moreover, the primers may comprise additional sequences, such as, for example, flag-tag sequences, kozac sequences, or other sequences discussed and/or referenced herein.

For example, in the case of the P155 to c691 N-terminal deletion mutant, the following primers could be used to amplify a cDNA fragment corresponding to this deletion mutant:

| | | |
|---|---|---|
| 5′ Primer | 5′-GCAGCA GCGGCCGC ACTGAGATAGTGGGAAAAGG-3′ | (SEQ ID NO:607) |
| | [symbol] | |
| 3′ Primer | 5′-GCAGCA GTCGAC TTAACAATGTGTTTCACTATCTGCC-3′ | (SEQ ID NO:608) |
| | [symbol] | |

For example, in the case of the M1 to K600 C-terminal deletion mutant, the following primers could be used to amplify a cDNA fragment corresponding to this deletion mutant:

| | | |
|---|---|---|
| 5′ Primer | 5′-GCAGCA GCGGCCGC ATGGACCAAAATCAACATTTG-3′ | (SEQ ID NO:609) |
| | [symbol] | |
| 3′ Primer | 5′-GCAGCA GTCGAC TATACACGTTGTATCAATCAGAGCC-3′ | (SEQ ID NO:610) |
| | [symbol] | |

Representative PCR amplification conditions are provided below, although the skilled artisan would appreciate that other conditions may be required for efficient amplification. A 100 ul PCR reaction mixture may be prepared using 10 ng of the template DNA (cDNA clone of an OATP2 or cMOAT polypeptide described herein), 200 uM 4 dNTPs, 1 uM primers, 0.25 U Taq DNA polymerase (PE), and standard Taq DNA polymerase buffer. Typical PCR cycling condition are as follows:

| | |
|---|---|
| 20-25 cycles: | 45 sec, 93 degrees |
| | 2 min, 50 degrees |
| | 2 min, 72 degrees |
| 1 cycle: | 10 min, 72 degrees |

After the final extension step of PCR, 5 U Klenow Fragment may be added and incubated for 15 min at 30 degrees.

Upon digestion of the fragment with the NotI and SalI restriction enzymes, the fragment could be cloned into an appropriate expression and/or cloning vector which has been similarly digested (e.g., pSport1, among others). The skilled artisan would appreciate that other plasmids could be equally substituted, and may be desirable in certain circumstances. The digested fragment and vector are then ligated using a DNA ligase, and then used to transform competent E.coli cells using methods provided herein and/or otherwise known in the art.

The 5′ primer sequence for amplifying any additional N-terminal deletion mutants may be determined by reference to the following formula:

(S+(X*3)) to ((S+(X*3))+25), wherein ‘S’ is equal to the nucleotide position of the initiating start codon of the OATP2 (SNP ID:PS100s2) gene (SEQ ID NO:7), and ‘X’ is equal to the most N-terminal amino acid of the intended N-terminal deletion mutant. The first term will provide the start 5′ nucleotide position of the 5′ primer, while the second term will provide the end 3′ nucleotide position of the 5′ primer corresponding to sense strand of SEQ ID NO:7. Once the corresponding nucleotide positions of the primer are determined, the final nucleotide sequence may be created by the addition of applicable restriction site sequences to the 5′ end of the sequence, for example. As referenced herein, the addition of other sequences to the 5′ primer may be desired in certain circumstances (e.g., kozac sequences, etc.).

The 3′ primer sequence for amplifying any additional N-terminal deletion mutants may be determined by reference to the following formula:

(S+(X*3)) to ((S+(X*3))−25), wherein ‘S’ is equal to the nucleotide position of the initiating start codon of the OATP2 (SNP ID:PS100s2) gene (SEQ ID NO:7), and ‘X’ is equal to the most C-terminal amino acid of the intended N-terminal deletion mutant. The first term will provide the start 5′ nucleotide position of the 3′ primer, while the second term will provide the end 3′ nucleotide position of the 3′ primer corresponding to the anti-sense strand of SEQ ID NO:7. Once the corresponding nucleotide positions of the primer are determined, the final nucleotide sequence may be created by the addition of applicable restriction site sequences to the 5′ end of the sequence, for example. As referenced herein, the addition of other sequences to the 3′ primer may be desired in certain circumstances (e.g., stop codon sequences, etc.). The skilled artisan would appreciate that modifications of the above nucleotide positions may be necessary for optimizing PCR amplification.

The same general formulas provided above may be used in identifying the 5′ and 3′ primer sequences for amplifying any C-terminal deletion mutant of the present invention. Moreover, the same general formulas provided above may be used in identifying the 5′ and 3′ primer sequences for amplifying any combination of N-terminal and C-terminal deletion mutant of the present invention. The skilled artisan would appreciate that modifications of the above nucleotide positions may be necessary for optimizing PCR amplification.

### Example 30

### Additional Methods of Genotyping the SNPs of the Present Invention

The skilled artisan would acknowledge that there are a number of methods that may be employed for genotyping a SNP of the present invention, aside from the preferred methods described herein. The present invention encompasses the following non-limiting types of genotype assays: PCR-free genotyping methods, Single-step homogeneous methods, Homogeneous detection with fluorescence polarization, Pyrosequencing, “Tag” based DNA chip system, Bead-based methods, fluorescent dye chemistry, Mass spectrometry based genotyping assays, TaqMan genotype assays, Invader genotype assays, and microfluidic genotype assays, among others.

Specifically encompassed by the present invention are the following, non-limiting genotyping methods: Landegren, U., Nilsson, M. & Kwok, P. Genome Res 8, 769-776 (1998); Kwok, P., Pharmacogenomics 1, 95-100 (2000); Gut, I., Hum Mutat 17, 475-492 (2001); Whitcombe, D., Newton, C. & Little, S., Curr Opin Biotechnol 9, 602-608 (1998); Tillib, S. & Mirzabekov, A., Curr Opin Biotechnol 12, 53-58 (2001); Winzeler, E. et al., Science 281, 1194-1197 (1998); Lyamichev, V. et al., Nat Biotechnol 17, 292-296 (1999); Hall, J. et al., Proc Natl Acad Sci USA 97, 8272-8277 (2000); Mein, C. et al., Genome Res 10, 333-343 (2000); Ohnishi, Y. et al., J Hum Genet 46, 471-477 (2001); Nilsson, M. et al., Science 265, 2085-2088 (1994); Baner, J., Nilsson, M., Mendel-Hartvig, M. & Landegren, U., Nucleic Acids Res 26, 5073-5078 (1998); Baner, J. et al., Curr Opin Biotechnol 12, 11-15 (2001); Hatch, A., Sano, T., Misasi, J. & Smith, C., Genet Anal 15, 35-40 (1999); Lizardi, P. et al., Nat Genet 19, 225-232 (1998); Zhong, X., Lizardi, P., Huang, X., Bray-Ward, P. & Ward, D., Proc Natl Acad Sci USA 98, 3940-3945 (2001); Faruqi, F. et al. BMC Genomics 2, 4 (2001); Livak, K., Gnet Anal 14, 143-149 (1999); Marras, S., Kramer, F. & Tyagi, S., Genet Anal 14, 151-156 (1999); Ranade, K. et al., Genome Res 11, 1262-1268 (2001); Myakishev, M., Khripin, Y., Hu, S. & Hamer, D., Genome Re 11, 163-169 (2001); Beaudet, L., Bedard, J., Breton, B., Mercuri, R. & Budarf, M., Genome Res 11, 600r608 (2001); Chen, X., Levine, L. & P Y, K., Genome Res 9, 492-498 (1999); Gibson, N. et al., Clin Chem 43, 1336-1341 (1997); Latif, S., Bauer-Sardina, I., Ranade, K., Livak, K. & P Y, K., Genome Res 11, 436-440 (2001); Hsu, T., Law, S., Duan, S., Neri, B. & Kwok, P., Clin Chem 47, 1373-1377 (2001); Alderborn, A., Kristofferson, A. & Hammerling, U., Genome Res 10, 1249-1258 (2000); Ronaghi, M., Ublen, M. & Nyren, P., Science 281, 363, 365 (1998); Ronaghi, M., Genome Res 11, 3-11 (2001); Pease, A. et al., Proc Natl Acad Sci. USA 91, 5022-5026 (1994); Southern, E., Maskos, U. & Elder, J., Genomics 13, 1008-1017 (1993); Wang, D. et al., Science 280, 1077-1082 (1998); Brown, P. & Botstein, D., Nat Genet 21, 33-37 (1999); Cargill, M. et al. Nat Genet 22, 231-238 (1999); Dong, S. et al., Genome Res 11, 1418-1424 (2001); Halushka, M. et al., Nat Genet 22, 239-247 (1999); Hacia, J., Nat Genet 21, 42-47 (1999); Lipshutz, R., Fodor, S., Gingeras, T. & Lockhart, D., Nat Genet 21, 20-24 (1999); Sapolsky, R. et al., Genet Anal 14, 187-192 (1999); Tsuchihashi, Z. & Brown, P., J Virol 68, 5863 (1994); Herschlag, D., J Biol Chem 270, 20871-20874 (1995); Head, S. et al., Nucleic Acids Res 25, 5065-5071 (1997); Nikiforov, T. et al., Nucleic Acids Res 22, 4167-4175 (1994); Syvanen, A. et al., Genormics 12, 590-595 (1992); Shumaker, J., Metspalu, A. & Caskey, C., Hum Mutat 7, 346-354 (1996); Lindroos, K., Liljedahl, U., Raitio, M. & Syvanen, A., Nucleic Acids Res 29, E69-9 (2001); Lindblad-Toh, K. et al., Nat Genet 24, 381-386 (2000); Pastinen, T. et al., Genome Res 10, 1031-1042 (2000); Fan, J. et al., Genome Res 10, 853-860 (2000); Hirschhorn, J. et al., Proc Natl Acad Sci USA 97, 12164-12169 (2000); Bouchie, A., Nat Biotechnol 19, 704 (2001); Hensel, M. et al., Science 269, 400-403 (1995); Shoemaker, D., Lashkari, D., Morris, D., Mittmann, M. & Davis, R. Nat Genet 14, 450-456 (1996); Gerry, N. et al., J Mol Biol 292, 251-262 (1999); Ladner, D. et al., Lab Invest 81, 1079-1086 (2001); lannone, M. et al. Cytometry 39, 131-140 (2000); Fulton, R., McDade, R., Smith, P., Kienker, L. & Kettman, J. J., Clin Chem 43, 1749-1756 (1997); Armstrong, B., Stewart, M. & Mazumder, A., Cytometry 40, 102-108 (2000); Cai, H. et al., Genomics 69, 395 (2000); Chen, J. et al., Genome Res 10, 549-557 (2000); Ye, F. et al. Hum Mutat 17, 305-316 (2001); Michael, K., Taylor, L., Schultz, S. & Walt, D., Anal Chem 70, 1242-1248 (1998); Steemers, F., Ferguson, J. & Walt, D., Nat Biotechnol 18, 91-94 (2000); Chan, W. & Nie, S., Science 281, 2016-2018 (1998); Han, M., Gao, X., Su, J. & Nie, S., Nat Biotechnol 19, 631-635 (2001); Griffin, T. & Smith, L., Trends Biotechnol 18, 77-84 (2000); Jackson, P., Scholl, P. & Groopman, J., Mol Med Today 6, 271-276 (2000); Haff, L. & Smirnov, I., Genome Res 7, 378-388 (1997); Ross, P., Hall, L., Smirnov, I. & Haff, L., Nat Biotechnol 16, 1347-1351 (1998); Bray, M., Boerwinkle, E. & Doris, P. Hum Mutat 17, 296-304 (2001); Sauer, S. et al., Nucleic Acids Res 28, E13 (2000); Sauer, S. et al., Nucleic Acids Res 28, E100 (2000); Sun, X., Ding, H., Hung, K. & Guo, B., Nucleic Acids Res 28, E68 (2000); Tang, K. et al., Proc Natl Acad Sci USA 91, 10016-10020 (1999); Li, J. et al., Electrophoresis 20, 1258-1265 (1999); Little, D., Braun, A., O'Donnell, M. & Koster, H., Nat Med 3, 1413-1416 (1997); Little, D. et al. Anal Chem 69, 4540-4546 (1997); Griffin, T., Tang, W. & Smith, L., Nat Biotechnol 15, 1368-1372 (1997); Ross, P., Lee, K. & Belgrader, P., Anal Chem 69, 4197-4202 (1997); Jiang-Baucom, P., Girard, J., Butler, J. & Belgrader, P., Anal Chem 69, 4894-4898 (1997); Griffin, T., Hall, J., Prudent, J. & Smith, L., Proc Natl Acad Sci USA 96, 6301-6306 (1999); Kokoris, M. et al., Mol Diagn 5, 329-340 (2000); Jurinke, C., van den Boom, D., Cantor, C. & Koster, H. (2001); and/or Taranenko, N. et al., Genet Anal 13, 87-94 (1996).

It will be clear that the invention may be practiced otherwise than as particularly described in the foregoing description and examples. Numerous modifications and variations of the present invention are possible in light of the above teachings and, therefore, are within the scope of the appended claims.

The entire disclosure of each document cited (including patents, patent applications, journal articles, abstracts, laboratory manuals, books, or other disclosures) in the Background of the Invention, Detailed Description, and Examples is hereby incorporated herein by reference. Further, the hard copy of the sequence listing submitted herewith and the corresponding computer readable form are both incorporated herein by reference in their entireties.

While this invention has been particularly shown and described with references to preferred embodiments thereof, it will be understood by those skilled in the art that various changes in form and details may be made therein without departing from the scope of the invention encompassed by the appended claims.

TABLE III

| | Coriell | Total | | | | |
|---|---|---|---|---|---|---|
| Gene name | DNA Panel | SNPs | Missense | Silent | UTR | non-CDS |
| OATP2 | 24 | 36 | 5 | 4 | 0 | 27 |
| cMOAT | 24 | 33 | 6 | 7 | 0 | 20 |
| Total: | | 69 | 11 | 11 | 0 | 47 |

TABLE IV

| | | | CONTIG_{—} | CONTIG_{—} | |
|---|---|---|---|---|---|
| GENE_DESCRIPTION | HGNC_ID | SNP_ID | NUM | POS | FLANK SEQ (REF/ALT) |
| OATP2, solute carrier family 21 member 6 | SLC21A6 | PS100s1 | 4 | 208 | TTCAACATC(A/G)ACCTTATCC |
| OATP2, solute carrier family 21 member 6 | SLC21A6 | PS100s2 | 4 | 260 | AGAGCATCA(C/A)CTGAGATAG |
| OATP2, solute carrier family 21 member 6 | SLC21A6 | PS100s3 | 4 | 438 | TACACAGTT(C/T)GCCCATTAA |
| OATP2, solute carrier family 21 member 6 | SLC21A6 | PS100s4 | 4 | 443 | AGTTCGCCC(T/A)TTAACAACA |
| OATP2, solute carrier family 21 member 6 | SLC21A6 | PS100s5 | 4 | 467 | TTAAACTAC(A/G)CGTTTTCAC |
| OATP2, solute carrier family 21 member 6 | SLC21A6 | PS100s6 | 4 | 469 | AAACTACGC(G/A)TTTTCACTT |
| OATP2, solute carrier family 21 member 6 | SLC21A6 | PS100s7 | 8 | 197 | AACTGTAGG(C/T)AGAAAAAA |
| OATP2, solute carrier family 21 member 6 | SLC21A6 | PS100s8 | 9 | 214 | TAATCTTA(C/A)CTTTTCCCA |
| OATP2, solute carrier family 21 member 6 | SLC21A6 | PS100s9 | 9 | 308 | TGATGAAT(C/T)GATATTAGT |
| OATP2, solute carrier family 21 member 6 | SLC21A6 | PS100s10 | 11 | 255 | TAAGAAA(T/A)TTTCACGG |
| OATP2, solute carrier family 21 member 6 | SLC21A6 | PS100s11 | 11 | 264 | TTTCACGG(G/A)AGAAGATT |
| OATP2, solute carrier family 21 member 6 | SLC21A6 | PS100s12 | 12 | 223 | ATTATTTTT(C/T)CTTTGACT |
| OATP2, solute carrier family 21 member 6 | SLC21A6 | PS100s13 | 13 | 345 | TGAAGCAT(A/G)TATTGAAAT |
| OATP2, solute carrier family 21 member 6 | SLC21A6 | PS100s14 | 13 | 420 | TCTTTGGC(A/G)AAATTTTTG |
| OATP2, solute carrier family 21 member 6 | SLC21A6 | PS100s15 | 13 | 430 | AAATTTTTG(A/T)TGCTTAATA |
| OATP2, solute carrier family 21 member 6 | SLC21A6 | PS100s16 | 13 | 434 | TTTTGATGC(C/T)TAATAGTTT |
| OATP2, solute carrier family 21 member 6 | SLC21A6 | PS100s17 | 13 | 435 | TTTGATGCT(A/T)AATAGTTTA |
| OATP2, solute carrier family 21 member 6 | SLC21A6 | PS100s18 | 13 | 475 | TATTTTGAT(G/A)GCTTCTCTT |
| OATP2, solute carrier family 21 member 6 | SLC21A6 | PS100s19 | 14 | 255 | TCAACTGGG(G/A)TAAATGTAT |
| OATP2, solute carrier family 21 member 6 | SLC21A6 | PS100s20 | 14 | 261 | GGGGTAAAT(G/T)TATCTCTCA |
| OATP2, solute carrier family 21 member 6 | SLC21A6 | PS100s21 | 14 | 396 | CTATAATGC(A/G)CAAAGAATG |
| OATP2, solute carrier family 21 member 6 | SLC21A6 | PS100s22 | 14 | 425 | AAATGTTGA(C/T)AGTGAGGAT |
| OATP2, solute carrier family 21 member 6 | SLC21A6 | PS100s23 | 16 | 244 | GATTTGCAA(C/G)CTGCTAGAC |
| OATP2, solute carrier family 21 member 6 | SLC21A6 | PS100s24 | 16 | 394 | GATAGA(C/G)ATATATCA |
| OATP2, solute carrier family 21 member 6 | SLC21A6 | PS100s25 | 17 | 133 | CAATGACAT(T/C)ACAGCAGT |
| OATP2, solute carrier family 21 member 6 | SLC21A6 | PS100s26 | 17 | 181 | GTTCAGTTT(C/G)AATTTTTTA |
| OATP2, solute carrier family 21 member 6 | SLC21A6 | PS100s27 | 17 | 380 | AGTTCTGGG(A/G)TACATGTG |
| OATP2, solute carrier family 21 member 6 | SLC21A6 | PS100s28 | 18 | 251 | ATGCATGA(C/G)ACTATAA |
| OATP2, solute carrier family 21 member 6 | SLC21A6 | PS100s29 | 19 | 75 | GGATATATG(T/C)GTTCATGGG |
| OATP2, solute carrier family 21 member 6 | SLC21A6 | PS100s30 | 19 | 125 | ATAGTACCA(T/C)TGGGGCTTT |
| OATP2, solute carrier family 21 member 6 | SLC21A6 | PS100s31 | 19 | 151 | TGATGATTT(C/T)GCTAAAGAA |
| OATP2, solute carrier family 21 member 6 | SLC21A6 | PS100s32 | 19 | 513 | CCATGATAA(C/T)GTCTTTCTA |
| OATP2, solute carrier family 21 member 6 | SLC21A6 | PS100s33 | 20 | 156 | TGTAGCATA(C/T)ATCCAAGCT |
| OATP2, solute carrier family 21 member 6 | SLC21A6 | PS100s34 | 20 | 170 | AAGCTAGAC(G/T)TCAGGGCCTT |
| OATP2, solute carrier family 21 member 6 | SLC21A6 | PS100s35 | 20 | 180 | TCAGGCCTT(A/C)GTATTATA |
| OATP2, solute carrier family 21 member 6 | SLC21A6 | PS100s36 | 20 | 291 | CCTGTATGA(C/T)ACTCATTTG |
| cMOAT, ATP-binding cassette sub-family C member 2 | ABCC2 | PS101s1 | 4 | 284 | ACAATGAGG(A/T)GAGGATTGA |
| cMOAT, ATP-binding cassette sub-family C member 2 | ABCC2 | PS101s2 | 6 | 213 | TTGTGACAT(T/C)GGTAGCATG |
| cMOAT, ATP-binding cassette sub-family C member 2 | ABCC2 | PS101s3 | 7 | 53 | AGTAAGTT(C/G)TAGAGCTCA |
| cMOAT, ATP-binding cassette sub-family C member 2 | ABCC2 | PS101s4 | 7 | 100 | AGGTCGTG(G/A)AGCCCAATG |
| cMOAT, ATP-binding cassette sub-family C member 2 | ABCC2 | PS101s5 | 7 | 148 | TGACCACC(A/G)GCAGCCTC |
| cMOAT, ATP-binding cassette sub-family C member 2 | ABCC2 | PS101s6 | 9 | 112 | GATCTAGA(G/A)ACAGACAA |
| cMOAT, ATP-binding cassette sub-family C member 2 | ABCC2 | PS101s7 | 9 | 190 | CAGGCTGCA(C/T)ACCATCATG |
| cMOAT, ATP-binding cassette sub-family C member 2 | ABCC2 | PS101s8 | 9 | 222 | GAGTGTAGG(A/G)GGACAGGGC |
| cMOAT, ATP-binding cassette sub-family C member 2 | ABCC2 | PS101s9 | 9 | 284 | GCCGGGAAA(C/T)ACCTGATGG |
| cMOAT, ATP-binding cassette sub-family C member 2 | ABCC2 | PS101s10 | 13 | 264 | CCAGCAGC(T/G)ATTTTCTGA |
| cMOAT, ATP-binding cassette sub-family C member 2 | ABCC2 | PS101s11 | 15 | 192 | ACTGGAA(A/G)GGTGAAC |
| cMOAT, ATP-binding cassette sub-family C member 2 | ABCC2 | PS101s12 | 15 | 204 | AACACCA(T/C)ACAGAA |
| cMOAT, ATP-binding cassette sub-family C member 2 | ABCC2 | PS101s13 | 16 | 859 | TCTGTGAG(C/T)ACAAGGGC |
| cMOAT, ATP-binding cassette sub-family C member 2 | ABCC2 | PS101s14 | 17 | 387 | AAGCAGCAT(A/T)GTGCTAAGT |
| cMOAT, ATP-binding cassette sub-family C member 2 | ABCC2 | PS101s15 | 18 | 86 | AGGGGCTGA(G/A)AACCTTTGC |
| cMOAT, ATP-binding cassette sub-family C member 2 | ABCC2 | PS101s16 | 18 | 172 | CCCCATG(C/A)ATTCTATTA |
| cMOAT, ATP-binding cassette sub-family C member 2 | ABCC2 | PS101s17 | 19 | 345 | TAGATC(C/T)CTGTCAG |
| cMOAT, ATP-binding cassette sub-family C member 2 | ABCC2 | PS101s18 | 19 | 367 | TGAACAAGG(A/G)GAGAGAAGC |
| cMOAT, ATP-binding cassette sub-family C member 2 | ABCC2 | PS101s19 | 20 | 281 | AGAGTCTT(T/C)GTTCCAGAC |
| cMOAT, ATP-binding cassette sub-family C member 2 | ABCC2 | PS101s20 | 21 | 243 | TTCTGTGCC(C/T)ATGATGATT |
| cMOAT, ATP-binding cassette sub-family C member 2 | ABCC2 | PS101s21 | 25 | 285 | CCCCATGCC(A/G)CTTTTCCTC |
| cMOAT, ATP-binding cassette sub-family C member 2 | ABCC2 | PS101s22 | 29 | 136 | ACAGGGCCT(G/A)TCACAGCCA |
| cMOAT, ATP-binding cassette sub-family C member 2 | ABCC2 | PS101s23 | 30 | 545 | TTCTCCAA(C/T)GGTGTACTC |
| cMOAT, ATP-binding cassette sub-family C member 2 | ABCC2 | PS101s24 | 30 | 575 | AGTTGGATA(A/G)GGTCAATGC |
| cMOAT, ATP-binding cassette sub-family C member 2 | ABCC2 | PS101s25 | 31 | 132 | AGCCTGGGG(G/T)TCTCAGCCT |
| cMOAT, ATP-binding cassette sub-family C member 2 | ABCC2 | PS101s26 | 33 | 27 | CTCCTGGGT(G/A)TGTCAACAT |
| cMOAT, ATP-binding cassette sub-family C member 2 | ABCC2 | PS101s27 | 33 | 54 | ATGGCAAAG(A/C)TGCTGAGGG |
| cMOAT, ATP-binding cassette sub-family C member 2 | ABCC2 | PS101s28 | 33 | 63 | CTGCTGAGG(A/G)GAGGAGTGA |
| cMOAT, ATP-binding cassette sub-family C member 2 | ABCC2 | PS101s29 | 34 | 615 | ACTAACCGA(A/G)GACTGAGGG |
| cMOAT, ATP-binding cassette sub-family C member 2 | ABCC2 | PS101s30 | 35 | 650 | TGGTAAA(C/T)CAATATCTA |
| cMOAT, ATP-binding cassette sub-family C member 2 | ABCC2 | PS101s31 | 35 | 742 | GGGTTCTGT(G/C)TCTCTTTGA |
| cMOAT, ATP-binding cassette sub-family C member 2 | ABCC2 | PS101s32 | 36 | 164 | ATTGTTTTC(G/A)ATATTCTTC |
| cMOAT, ATP-binding cassette sub-family C member 2 | ABCC2 | PS101s33 | 36 | 690 | TCTTTCTGA(C/T)AGGGAGGA |

| | FLANK_{—} | FLANK_{—} | | REF_{—} | | | |
|---|---|---|---|---|---|---|---|
| | SEQ REF | SEQ ALT | | SEQ_{—} | | ALT_{—} | |
| GENE_DESCRIPTION | (SEQ ID NO) | (SEQ ID NO) | REF_SEQ_ID | POS | REF_NT | NT | EXON |
| OATP2, solute carrier family 21 member 6 | 51 | 120 | AC022335 8 | 82621 | C | T | Exon5 |
| OATP2, solute carrier family 21 member 6 | 52 | 121 | AC022335 8 | 82569 | G | T | Exon5 |
| OATP2, solute carrier family 21 member 6 | 53 | 122 | AC022335 8 | 82391 | G | A | Intron5 |
| OATP2, solute carrier family 21 member 6 | 54 | 123 | AC022335 8 | 82386 | T | A | Intron5 |
| OATP2, solute carrier family 21 member 6 | 55 | 124 | AC022335 8 | 82362 | C | T | Intron5 |
| OATP2, solute carrier family 21 member 6 | 56 | 125 | AC022335 8 | 82360 | C | T | Intron5 |
| OATP2, solute carrier family 21 member 6 | 57 | 126 | AC022335 8 | 20092 | A | G | Exon15 |
| OATP2, solute carrier family 21 member 6 | 58 | 127 | AC022335 8 | 82550 | C | A | Intron5 |
| OATP2, solute carrier family 21 member 6 | 59 | 128 | AC022335 8 | 82644 | T | C | Exon5 |
| OATP2, solute carrier family 21 member 6 | 60 | 129 | AC022335 8 | 128161 | A | T | Intron1 |
| OATP2, solute carrier family 21 member 6 | 61 | 130 | AC022335 8 | 128152 | T | C | Intron1 |
| OATP2, solute carrier family 21 member 6 | 62 | 131 | AC022335 8 | 34576 | A | G | Intron14 |
| OATP2, solute carrier family 21 member 6 | 63 | 132 | AC022335 8 | 117672 | T | C | Intron2 |
| OATP2, solute carrier family 21 member 6 | 64 | 133 | AC022335 8 | 117597 | C | T | Intron2 |
| OATP2, solute carrier family 21 member 6 | 65 | 134 | AC022335 8 | 117587 | T | A | Intron2 |
| OATP2, solute carrier family 21 member 6 | 66 | 135 | AC022335 8 | 117583 | A | G | Intron2 |
| OATP2, solute carrier family 21 member 6 | 67 | 136 | AC022335 8 | 117582 | A | T | Intron2 |
| OATP2, solute carrier family 21 member 6 | 68 | 137 | AC022335 8 | 117542 | T | C | Intron2 |
| OATP2, solute carrier family 21 member 6 | 69 | 138 | AC022335 8 | 86574 | C | T | Intron3 |
| OATP2, solute carrier family 21 member 6 | 70 | 139 | AC022335 8 | 86588 | A | C | Intron3 |
| OATP2, solute carrier family 21 member 6 | 71 | 140 | AC022335 8 | 86433 | T | C | Intron3 |
| OATP2, solute carrier family 21 member 6 | 72 | 141 | AC022335 8 | 86404 | A | G | Intron3 |
| OATP2, solute carrier family 21 member 6 | 73 | 142 | AC022335 8 | 53449 | C | G | Exon11 |
| OATP2, solute carrier family 21 member 6 | 74 | 143 | AC022335 8 | 53599 | C | G | Intron10 |
| OATP2, solute carrier family 21 member 6 | 75 | 144 | AC022335 8 | 56845 | C | T | Exon10 |
| OATP2, solute carrier family 21 member 6 | 76 | 145 | AC022335 8 | 56893 | G | C | Exon10 |
| OATP2, solute carrier family 21 member 6 | 77 | 146 | AC022335 8 | 57092 | A | G | Intron9 |
| OATP2, solute carrier family 21 member 6 | 78 | 147 | AC022335 8 | 42107 | C | G | Intron12 |
| OATP2, solute carrier family 21 member 6 | 79 | 148 | AC022335 8 | 80833 | A | G | Exon6 |
| OATP2, solute carrier family 21 member 6 | 80 | 149 | AC022335 8 | 80783 | A | G | Exon6 |
| OATP2, solute carrier family 21 member 6 | 81 | 150 | AC022335 8 | 80757 | G | A | Exon6 |
| OATP2, solute carrier family 21 member 6 | 82 | 151 | AC022335 8 | 80395 | G | A | Intron7 |
| OATP2, solute carrier family 21 member 6 | 83 | 152 | AC022335 8 | 19796 | T | C | Exon15 |
| OATP2, solute carrier family 21 member 6 | 84 | 153 | AC022335 8 | 19810 | T | G | Exon15 |
| OATP2, solute carrier family 21 member 6 | 85 | 154 | AC022335 8 | 19820 | A | C | Exon15 |
| OATP2, solute carrier family 21 member 6 | 86 | 155 | AC022335 8 | 19931 | T | C | Exon15 |
| cMOAT, ATP-binding cassette sub-family C member 2 | 87 | 156 | AL392107 4 | 66274 | A | T | Exon25 |
| cMOAT, ATP-binding cassette sub-family C member 2 | 88 | 157 | AL392107 4 | 58303 | G | A | Exon28 |
| cMOAT, ATP-binding cassette sub-family C member 2 | 89 | 158 | AL392107 4 | 56960 | C | G | Intron29 |
| cMOAT, ATP-binding cassette sub-family C member 2 | 90 | 159 | AL392107 4 | 57007 | G | A | Exon29 |
| cMOAT, ATP-binding cassette sub-family C member 2 | 91 | 160 | AL392107 4 | 57055 | G | A | Exon29 |
| cMOAT, ATP-binding cassette sub-family C member 2 | 92 | 161 | AL392107 4 | 52055 | C | T | Exon31 |
| cMOAT, ATP-binding cassette sub-family C member 2 | 93 | 162 | AL392107 4 | 51977 | G | A | Exon31 |
| cMOAT, ATP-binding cassette sub-family C member 2 | 94 | 163 | AL392107 4 | 51945 | C | T | Intron31 |
| cMOAT, ATP-binding cassette sub-family C member 2 | 95 | 164 | AL392107 4 | 51883 | G | A | Intron31 |
| cMOAT, ATP-binding cassette sub-family C member 2 | 96 | 165 | AL392107 4 | 66295 | C | A | Exon25 |
| cMOAT, ATP-binding cassette sub-family C member 2 | 97 | 166 | AL392107 4 | 71663 | T | C | Exon21 |
| cMOAT, ATP-binding cassette sub-family C member 2 | 98 | 167 | AL392107 4 | 71651 | G | A | Intron21 |
| cMOAT, ATP-binding cassette sub-family C member 2 | 99 | 168 | AL392107 4 | 110259 | T | C | Exon3 |
| cMOAT, ATP-binding cassette sub-family C member 2 | 100 | 169 | AL392107 4 | 94484 | T | A | Intron12 |
| cMOAT, ATP-binding cassette sub-family C member 2 | 101 | 170 | AL392107 4 | 90742 | G | A | Intron15 |
| cMOAT, ATP-binding cassette sub-family C member 2 | 102 | 171 | AL392107 4 | 90828 | C | A | Intron15 |
| cMOAT, ATP-binding cassette sub-family C member 2 | 103 | 172 | AL392107 4 | 72250 | C | T | Intron19 |
| cMOAT, ATP-binding cassette sub-family C member 2 | 104 | 173 | AL392107 4 | 72272 | A | G | Intron19 |
| cMOAT, ATP-binding cassette sub-family C member 2 | 105 | 174 | AL392107 4 | 119722 | G | A | Exon1 |
| cMOAT, ATP-binding cassette sub-family C member 2 | 106 | 175 | AL392107 4 | 58988 | A | G | Intron26 |
| cMOAT, ATP-binding cassette sub-family C member 2 | 107 | 176 | AL392107 4 | 94844 | C | T | Intron12 |
| cMOAT, ATP-binding cassette sub-family C member 2 | 108 | 177 | AL392107 4 | 89454 | A | G | Exon16 |
| cMOAT, ATP-binding cassette sub-family C member 2 | 109 | 178 | AL392107 4 | 98465 | C | T | Exon10 |
| cMOAT, ATP-binding cassette sub-family C member 2 | 110 | 179 | AL392107 4 | 98495 | G | A | Exon10 |
| cMOAT, ATP-binding cassette sub-family C member 2 | 111 | 180 | AL392107 4 | 105455 | C | A | Intron6 |
| cMOAT, ATP-binding cassette sub-family C member 2 | 112 | 181 | AL392107 4 | 67834 | A | G | Intron24 |
| cMOAT, ATP-binding cassette sub-family C member 2 | 113 | 182 | AL392107 4 | 67861 | C | A | Intron24 |
| cMOAT, ATP-binding cassette sub-family C member 2 | 114 | 183 | AL392107 4 | 67870 | G | A | Intron24 |
| cMOAT, ATP-binding cassette sub-family C member 2 | 115 | 184 | AL392107 4 | 109039 | A | G | Intron3 |
| cMOAT, ATP-binding cassette sub-family C member 2 | 116 | 185 | AL392107 4 | 83188 | A | G | Intron19 |
| cMOAT, ATP-binding cassette sub-family C member 2 | 117 | 186 | AL392107 4 | 83096 | C | G | Intron19 |
| cMOAT, ATP-binding cassette sub-family C member 2 | 118 | 187 | AL392107 4 | 70852 | C | T | Exon22 |
| cMOAT, ATP-binding cassette sub-family C member 2 | 119 | 188 | AL392107 4 | 70326 | A | G | Intron23 |

| | | REV_{—} | REF_{—} | ALT_{—} | CDNA_SEQ_{—} | |
|---|---|---|---|---|---|---|
| GENE_DESCRIPTION | MUTATION TYPE | COMP | CODON | CODON | ID | CDNA_SEQ_POS |
| OATP2, solute carrier family 21 member 6 | Silent | 1 | TCG | TCA | AF205071 1 | 545 |
| OATP2, solute carrier family 21 member 6 | Missense | 1 | CCT | ACT | AF205071 1 | 597 |
| OATP2, solute carrier family 21 member 6 | Non-CDS | 1 | | | AF205071 1 | |
| OATP2, solute carrier family 21 member 6 | Non-CDS | 1 | | | AF205071 1 | |
| OATP2, solute carrier family 21 member 6 | Non-CDS | 1 | | | AF205071 1 | |
| OATP2, solute carrier family 21 member 6 | Non-CDS | 1 | | | AF205071 1 | |
| OATP2, solute carrier family 21 member 6 | Non-CDS | 1 | | | AF205071 1 | 2377 |
| OATP2, solute carrier family 21 member 6 | Non-CDS | 1 | | | AF205071 1 | |
| OATP2, solute carrier family 21 member 6 | Missense | 1 | GAT | TAT | AF205071 1 | 522 |
| OATP2, solute carrier family 21 member 6 | Non-CDS | 1 | | | AF205071 1 | |
| OATP2, solute carrier family 21 member 6 | Non-CDS | 1 | | | AF205071 1 | |
| OATP2, solute carrier family 21 member 6 | Non-CDS | 1 | | | AF205071 1 | |
| OATP2, solute carrier family 21 member 6 | Non-CDS | 1 | | | AF205071 1 | |
| OATP2, solute carrier family 21 member 6 | Non-CDS | 1 | | | AF205071 1 | |
| OATP2, solute carrier family 21 member 6 | Non-CDS | 1 | | | AF205071 1 | |
| OATP2, solute carrier family 21 member 6 | Non-CDS | 1 | | | AF205071 1 | |
| OATP2, solute carrier family 21 member 6 | Non-CDS | 1 | | | AF205071 1 | |
| OATP2, solute carrier family 21 member 6 | Non-CDS | 1 | | | AF205071 1 | |
| OATP2, solute carrier family 21 member 6 | Non-CDS | 1 | | | AF205071 1 | |
| OATP2, solute carrier family 21 member 6 | Non-CDS | 1 | | | AF205071 1 | |
| OATP2, solute carrier family 21 member 6 | Non-CDS | 1 | | | AF205071 1 | |
| OATP2, solute carrier family 21 member 6 | Non-CDS | 1 | | | AF205071 1 | |
| OATP2, solute carrier family 21 member 6 | Missense | 1 | GGT | GCT | AF205071 1 | 1597 |
| OATP2, solute carrier family 21 member 6 | Non-CDS | 1 | | | AF205071 1 | |
| OATP2, solute carrier family 21 member 6 | Silent | 1 | GTG | GTA | AF205071 1 | 1382 |
| OATP2, solute carrier family 21 member 6 | Missense | 1 | TTC | TTG | AF205071 1 | 1334 |
| OATP2, solute carrier family 21 member 6 | Non-CDS | 1 | | | AF205071 1 | |
| OATP2, solute carrier family 21 member 6 | Non-CDS | 1 | | | AF205071 1 | |
| OATP2, solute carrier family 21 member 6 | Missense | 1 | GTG | GCG | AF205071 1 | 655 |
| OATP2, solute carrier family 21 member 6 | Silent | 1 | TTG | CTG | AF205071 1 | 705 |
| OATP2, solute carrier family 21 member 6 | Silent | 1 | TTC | TTT | AF205071 1 | 731 |
| OATP2, solute carrier family 21 member 6 | Non-CDS | 1 | | | AF205071 1 | |
| OATP2, solute carrier family 21 member 6 | Non-CDS | 1 | | | AF205071 1 | 2673 |
| OATP2, solute carrier family 21 member 6 | Non-CDS | 1 | | | AF205071 1 | 2659 |
| OATP2, solute carrier family 21 member 6 | Non-CDS | 1 | | | AF205071 1 | 2649 |
| OATP2, solute carrier family 21 member 6 | Non-CDS | 1 | | | AF205071 1 | 2538 |
| cMOAT, ATP-binding cassette sub-family C member 2 | Missense | 1 | GAG | GTG | U49248 1 | 3664 |
| cMOAT, ATP-binding cassette sub-family C member 2 | Silent | 1 | ATC | ATT | U49248 1 | 4073 |
| cMOAT, ATP-binding cassette sub-family C member 2 | Non-CDS | 1 | | | U49248 1 | |
| cMOAT, ATP-binding cassette sub-family C member 2 | Silent | 1 | CTC | CTT | U49248 1 | 4211 |
| cMOAT, ATP-binding cassette sub-family C member 2 | Silent | 1 | GCC | GCT | U49248 1 | 4163 |
| cMOAT, ATP-binding cassette sub-family C member 2 | Silent | 1 | GAG | GAA | U49248 1 | 4511 |
| cMOAT, ATP-binding cassette sub-family C member 2 | Silent | 1 | CAT | CAC | U49248 1 | 4589 |
| cMOAT, ATP-binding cassette sub-family C member 2 | Non-CDS | 1 | | | U49248 1 | |
| cMOAT, ATP-binding cassette sub-family C member 2 | Non-CDS | 1 | | | U49248 1 | |
| cMOAT, ATP-binding cassette sub-family C member 2 | Missense | 1 | CGA | CTA | U49248 1 | 3643 |
| cMOAT, ATP-binding cassette sub-family C member 2 | Missense | 1 | AAG | AGG | U49248 1 | 2983 |
| cMOAT, ATP-binding cassette sub-family C member 2 | Non-CDS | 1 | | | U49248 1 | |
| cMOAT, ATP-binding cassette sub-family C member 2 | Silent | 1 | GTA | GTG | U49248 1 | 359 |
| cMOAT, ATP-binding cassette sub-family C member 2 | Non-CDS | 1 | | | U49248 1 | |
| cMOAT, ATP-binding cassette sub-family C member 2 | Non-CDS | 1 | | | U49248 1 | |
| cMOAT, ATP-binding cassette sub-family C member 2 | Non-CDS | 1 | | | U49248 1 | |
| cMOAT, ATP-binding cassette sub-family C member 2 | Non-CDS | 1 | | | U49248 1 | |
| cMOAT, ATP-binding cassette sub-family C member 2 | Non-CDS | 1 | | | U49248 1 | |
| cMOAT, ATP-binding cassette sub-family C member 2 | Non-CDS | 1 | | | U49248 1 | 78 |
| cMOAT, ATP-binding cassette sub-family C member 2 | Non-CDS | 1 | | | U49248 1 | |
| cMOAT, ATP-binding cassette sub-family C member 2 | Non-CDS | 1 | | | U49248 1 | |
| cMOAT, ATP-binding cassette sub-family C member 2 | Missense | 1 | ATA | ACA | U49248 1 | 2110 |
| cMOAT, ATP-binding cassette sub-family C member 2 | Missense | 1 | GTT | ATT | U49248 1 | 1350 |
| cMOAT, ATP-binding cassette sub-family C member 2 | Missense | 1 | CTA | TTA | U49248 1 | 1320 |
| cMOAT, ATP-binding cassette sub-family C member 2 | Non-CDS | 1 | | | U49248 1 | |
| cMOAT, ATP-binding cassette sub-family C member 2 | Non-CDS | 1 | | | U49248 1 | |
| cMOAT, ATP-binding cassette sub-family C member 2 | Non-CDS | 1 | | | U49248 1 | |
| cMOAT, ATP-binding cassette sub-family C member 2 | Non-CDS | 1 | | | U49248 1 | |
| cMOAT, ATP-binding cassette sub-family C member 2 | Non-CDS | 1 | | | U49248 1 | |
| cMOAT, ATP-binding cassette sub-family C member 2 | Non-CDS | 1 | | | U49248 1 | |
| cMOAT, ATP-binding cassette sub-family C member 2 | Non-CDS | 1 | | | U49248 1 | |
| cMOAT, ATP-binding cassette sub-family C member 2 | Silent | 1 | TCG | TCA | U49248 1 | 3035 |
| cMOAT, ATP-binding cassette sub-family C member 2 | Non-CDS | 1 | | | U49248 1 | |

TABLE V

| | | | | | | | REFSEQ_{—} |
|---|---|---|---|---|---|---|---|
| | | | | | | | FLANK |
| | | | | | REFSEQ_{—} | | REF |
| | | | CONTIG_{—} | CONTIG_{—} | FLANK_{—} | | (SEQ |
| GENE_DESCRIPTION | HGNC_ID | SNP_ID | NUM | POS | ORIENT | REFSEC FLANK | ID NO) |
| OATP2, solute carrier family 21 member 6 | SLC21A6 | PS100s1 | 4 | 208 | 1 | ATTAAACAAGTGGATAAGGT(C/T)GATGTTGAATTTTCTGATGA | 189 |
| OATP2, solute carrier family 21 member 6 | SLC21A6 | PS100s2 | 4 | 260 | 1 | ACCTTTTCCCACTATCTCAG(G/T)TGATGCTCTATTGAGTGATA | 190 |
| OATP2, solute carrier family 21 member 6 | SLC21A6 | PS100s3 | 4 | 438 | 1 | AACCTGTGTTGTTAATGGGC(G/A)AACTGTGTATATTAACACTA | 191 |
| OATP2, solute carrier family 21 member 6 | SLC21A6 | PS100s4 | 4 | 443 | 1 | GTTTAAACCTGTGTTGTTAA(T/A)GGGCGAACTGTGTATATTAA | 192 |
| OATP2, solute carrier family 21 member 6 | SLC21A6 | PS100s5 | 4 | 467 | 1 | TTTGCATAGAAGTGAAAACG(C/T)GTAGTTTAAACCTGTGTTGT | 193 |
| OATP2, solute carrier family 21 member 6 | SLC21A6 | PS100s6 | 4 | 469 | 1 | AATTTGCATAGAAGTGAAAA(C/T)GCGTAGTTTAAACCTGTGTT | 194 |
| OATP2, solute carrier family 21 member 6 | SLC21A6 | PS100s7 | 8 | 197 | 1 | TGAGTACTCTCATTTTTTCT(A/G)CCTACAGTTTGTTTTATTAT | 195 |
| OATP2, solute carrier family 21 member 6 | SLC21A6 | PS100s8 | 9 | 214 | 0 | ACTGTCAATATTAATTCTTA(C/A)CTTTTCCCACTATCTCAGGT | 196 |
| OATP2, solute carrier family 21 member 6 | SLC21A6 | PS100s9 | 9 | 308 | 0 | TGTTGAATTTTCTGATGAAT(T/C)GATATTAGTTTCTTTAGAAT | 197 |
| OATP2, solute carrier family 21 member 6 | SLC21A6 | PS100s10 | 11 | 255 | 1 | CAAAATCTTCTTCCGTGAAA(A/T)TTTCTTACATAAAATATAG | 198 |
| OATP2, solute carrier family 21 member 6 | SLC21A6 | PS100s11 | 11 | 264 | 1 | CAAGACCATCAAAATCTT(T/C)CCGTGAAAATTTCTTACATA | 199 |
| OATP2, solute carrier family 21 member 6 | SLC21A6 | PS100s12 | 12 | 223 | 1 | AGGAATTAATATAGTCAAAG(A/G)AAAAATAATGAAATATATTT | 200 |
| OATP2, solute carrier family 21 member 6 | SLC21A6 | PS100s13 | 13 | 345 | 1 | ATTATGTTAATATTTCAATA(T/C)ATGCTTCAATTGAAAATTAT | 201 |
| OATP2, solute carrier family 21 member 6 | SLC21A6 | PS100s14 | 13 | 420 | 1 | CTATTAAGCATCAAAAATTT(C/T)GCCAAAGAAATACTTAATCA | 202 |
| OATP2, solute carrier family 21 member 6 | SLC21A6 | PS100s15 | 13 | 430 | 1 | CATTGATAAACTATTAAGCA(T/A)CAAAAATTTCGCCAAAGAAA | 203 |
| OATP2, solute carrier family 21 member 6 | SLC21A6 | PS100s16 | 13 | 434 | 1 | TCTACATTGATAAACTATTA(A/G)GCATCAAAAATTTCGCCAAA | 204 |
| OATP2, solute carrier family 21 member 6 | SLC21A6 | PS100s17 | 13 | 435 | 1 | TTCTACATTGATAAACTATT(A/T)AGCATCAAAAATTTCGCCAA | 205 |
| OATP2, solute carrier family 21 member 6 | SLC21A6 | PS100s18 | 13 | 475 | 1 | ATCCAAAACCAAAGAGAAGC(T/C)ATCAAATATTTCTAAATTT | 206 |
| OATP2, solute carrier family 21 member 6 | SLC21A6 | PS100s19 | 14 | 255 | 1 | GCCTGTGAGAGATAAATTTA(C/T)CCCAGTTGATAACCAGTGGT | 207 |
| OATP2, solute carrier family 21 member 6 | SLC21A6 | PS100s20 | 14 | 261 | 1 | CAAATTGCCTGTGAGAGATA(A/C)ATTTACCCCAGTTGTAACC | 208 |
| OATP2, solute carrier family 21 member 6 | SLC21A6 | PS100s21 | 14 | 396 | 1 | TTCAGTTACATCATTCTTTG(T/C)GCATTATAGAATATTTAGCA | 209 |
| OATP2, solute carrier family 21 member 6 | SLC21A6 | PS100s22 | 14 | 425 | 1 | GGTTTCTGAACATCCTCACT(A/G)TCAACATTTTCAGTTACATC | 210 |
| OATP2, solute carrier family 21 member 6 | SLC21A6 | PS100s23 | 16 | 244 | 0 | TGCCACTTGAAGATTTCCAA(C/G)CTGCTAGACAGGGTGAGATG | 211 |
| OATP2, solute carrier family 21 member 6 | SLC21A6 | PS100s24 | 16 | 384 | 0 | TTCTGGAAATATATGATAGA(C/G)ATATATCAAAAAGAGAGAGA | 212 |
| OATP2, solute carrier family 21 member 6 | SLC21A6 | PS100s25 | 17 | 133 | 0 | AGGTAAAAGGACAATGACAT(C/T)ACAGCAGTAAAACATGAGAA | 213 |
| OATP2, solute carrier family 21 member 6 | SLC21A6 | PS100s26 | 17 | 81 | 0 | ATTCCAACGGTGTTCAGTTT(G/C)AATTTTTTAATGATATATCC | 214 |
| OATP2, solute carrier family 21 member 6 | SLC21A6 | PS100s27 | 17 | 380 | 0 | ATTTTACTTTAAGTTCTGGG(A/G)TACATGTGCAGAATGTGCAT | 215 |
| OATP2, solute carrier family 21 member 6 | SLC21A6 | PS100s28 | 18 | 251 | 1 | TCATTAGGTGTGTTTATAGT(C/G)TCATGCATATGAAAATGTTT | 216 |
| OATP2, solute carrier family 21 member 6 | SLC21A6 | PS100s29 | 19 | 75 | 1 | GAAGCATATTACCCATGAAC(A/G)CATATATCCACATGTATGAC | 217 |
| OATP2, solute carrier family 21 member 6 | SLC21A6 | PS100s30 | 19 | 125 | 1 | ATCAATGTAAGAAAGCCCCA(A/G)TGGTACTATGGGAGTCTCCC | 218 |
| OATP2, solute carrier family 21 member 6 | SLC21A6 | PS100s31 | 19 | 151 | 1 | GAAGAATGTCCTTCTTTAGC(G/A)AAATCATCAATGTAAGAAAG | 219 |
| OATP2, solute carrier family 21 member 6 | SLC21A6 | PS100s32 | 18 | 513 | 1 | GCATGTGTGCTTAGAAAGAC(G/A)TTATCATGGTACCTTGTTCT | 220 |
| OATP2, solute carrier family 21 member 6 | SLC21A6 | PS100s33 | 20 | 156 | 0 | ACAGATATTATTGTAGCATA(T/C)TCCAAAGCTAGACTTCAGGC | 221 |
| OATP2, solute carrier family 21 member 6 | SLC21A6 | PS100s34 | 20 | 170 | 0 | AGCATATATCCAAGCTAGAC(T/G)TCAGGCCTTAGTATTATAGT | 222 |
| OATP2, solute carrier family 21 member 6 | SLC21A6 | PS100s35 | 20 | 180 | 0 | CAAGCTAGACTTCAGGCCTT(A/C)GTATTATAGTTCAAACTCTG | 223 |
| OATP2, solute carrier family 21 member 6 | SLC21A6 | PS100s36 | 20 | 291 | 0 | TTTAACCTCTAACCTGTATGA(T/C)ACTCATTTGTTTTATTAAGA | 224 |
| cMOAT, ATP-binding cassette sub-family C member 2 | ABCC2 | PS101s1 | 4 | 284 | 1 | TCTGGTTGGTGTCAATCCTC(A/T)CCTCATTGTGTTTCAGAAAT | 225 |
| cMOAT, ATP-binding cassette sub-family C member 2 | ABCC2 | PS101s2 | 6 | 213 | 1 | CCTACCTTCTCCATGCTACC(G/A)ATGTCACAAGTGATCCCTCT | 226 |
| cMOAT, ATP-binding cassette sub-family C member 2 | ABCC2 | PS101s3 | 7 | 53 | 0 | GGCATGTGCCCGAGTAAGTT(C/G)TAGAGCTCACCTGGGGGATG | 227 |
| cMOAT, ATP-binding cassette sub-family C member 2 | ABCC2 | PS101s4 | 7 | 100 | 0 | AGCTTCTCTCGGAGGTCGTG(G/A)GCASCCTCTAAGATTCTGAA | 228 |
| cMOAT, ATP-binding cassette sub-family C member 2 | ABCC2 | PS101s5 | 7 | 148 | 0 | TCAATGATAATCTGACCACC(G/A)GCASCCTCTAAGATTCTGAA | 229 |
| cMOAT, ATP-binding cassette sub-family C member 2 | ABCC2 | PS101s6 | 9 | 112 | 1 | GTCTGAATGAGGTTGTCTGT(C/T)CTAGATCCACCGCAGCAGT | 230 |
| cMOAT, ATP-binding cassette sub-family C member 2 | ABCC2 | PS101s7 | 9 | 190 | 1 | TTGTCACTGTCCATGATGGT(G/A)TGCAGCCTGTGGGCGATGGT | 231 |
| cMOAT, ATP-binding cassette sub-family C member 2 | ABCC2 | PS101s8 | 9 | 222 | 1 | ATCCGTGTCAAGCCCCTGTCC(C/T)CCTACACTCACTTGTCACTG | 232 |
| cMOAT, ATP-binding cassette sub-family C member 2 | ABCC2 | PS101s9 | 9 | 284 | 1 | TAACCTTCTGCCATCAGGT(G/A)TTTCCCGGCTGACACTGTTA | 233 |
| cMOAT, ATP-binding cassette sub-family C member 2 | ABCC2 | PS101s10 | 13 | 254 | 1 | CCTCATTGTGTTTCAGAAAT(C/A)GCTGCTGGTGCTCAAAGGCA | 234 |
| cMOAT, ATP-binding cassette sub-family C member 2 | ABCC2 | PS101s11 | 15 | 192 | 1 | TTTTCTGTGTGGTGTTCACC(T/C)TTCCAGTTTCTATGAATTCC | 235 |
| cMOAT, ATP-binding cassette sub-family C member 2 | ABCC2 | PS101s12 | 15 | 264 | 1 | CTCAAATGCTACTTTTCTGT(G/A)TGGTGTTCACCTTTCCAGTT | 236 |
| cMOAT, ATP-binding cassette sub-family C member 2 | ABCC2 | PS101s13 | 16 | 859 | 0 | TGTCCAGAGTCTTCTGTGAG(T/C)ACAAGGGCCAGCTCTATGGC | 237 |
| cMOAT, ATP-binding cassette sub-family C member 2 | ABCC2 | PS101s14 | 17 | 367 | 0 | TAAAAGGGACCAAGCAT(T/A)GTGCTAAGTTTCAGGAACAC | 238 |
| cMOAT, ATP-binding cassette sub-family C member 2 | ABCC2 | PS101s15 | 18 | 86 | 0 | TTATCAGGGAAHHHHCATGA(G/A)AACCTTTGCTTTCAGCTCAT | 239 |
| cMOAT, ATP-binding cassette sub-family C member 2 | ABCC2 | PS101s16 | 18 | 172 | 0 | ACATAGCTCATTTCCCCATG(C/A)ATTCTATTACATGGAATTTT | 240 |
| cMOAT, ATP-binding cassette sub-family C member 2 | ABCC2 | PS101s17 | 19 | 345 | 0 | GGGAAAGAGCTGCATAGATC(C/T)CTGTCAGTCCTATGAACAAG | 241 |
| cMOAT, ATP-binding cassette sub-family C member 2 | ABCC2 | PS101s18 | 19 | 367 | 0 | TGTCAGTCCTATGAACAAGG(A/G)GAGAGAASCCTCCTCTGATC | 242 |
| cMOAT, ATP-binding cassette sub-family C member 2 | ABCC2 | PS101s19 | 20 | 281 | 1 | TCCTGGACTGCGTCTGGAG(G/A)AAGACTCTTCTATTAATATG | 243 |
| cMOAT, ATP-binding cassette sub-family C member 2 | ABCC2 | PS101s20 | 21 | 243 | 1 | AGAAGACTGAAAATCATCA(A/G)GCACAGAAACCAACTCAAC | 244 |
| cMOAT, ATP-binding cassette sub-family C member 2 | ABCC2 | PS101s21 | 25 | 285 | 1 | ATGGTACAAAGGAGGAAAAG(C/T)GGCATGGGGCGGTGAGAGGG | 245 |
| cMOAT, ATP-binding cassette sub-family C member 2 | ABCC2 | PS101s22 | 29 | 136 | 0 | TCCAGAGCCGACAGGGCCT(A/G)TCACAGCCACAAGTTGGCCT | 246 |
| cMOAT, ATP-binding cassette sub-family C member 2 | ABCC2 | PS101s23 | 30 | 545 | 0 | CAGGTTCACTGTTTCTCCAA(C/T)GGTGTACTCCTTCCTGGCCA | 247 |
| cMOAT, ATP-binding cassette sub-family C member 2 | ABCC2 | PS101s24 | 30 | 575 | 0 | CTTCCTGGCCAAGTA(G/A)GGTCAATGCCTAAAGATAAG | 248 |
| cMOAT, ATP-binding cassette sub-family C member 2 | ABCC2 | PS101s25 | 31 | 132 | 1 | GAGCGAACCACAGGCTGAGA(C/A)CCCCAGGCTGGGTCAGAGGC | 249 |
| cMOAT, ATP-binding cassette sub-family C member 2 | ABCC2 | PS101s26 | 33 | 27 | 0 | TGGCCTGTGGGCTCCTGGGT(A/G)TGTCAACATATGACTAAATG | 250 |
| cMOAT, ATP-binding cassette sub-family C member 2 | ABCC2 | PS101s27 | 33 | 54 | 0 | CATATGACTAAATGGCAAAG(C/A)TGCTGAGGGGAGGAGTGAGT | 251 |
| cMOAT, ATP-binding cassette sub-family C member 2 | ABCC2 | PS101s28 | 33 | 63 | 0 | AAATGGCAAAGCTGCTGAGG(G/A)GAGGAGTGAGTGACTAGCAA | 252 |
| cMOAT, ATP-binding cassette sub-family C member 2 | ABCC2 | PS101s29 | 34 | 815 | 0 | AGAATACTGCCACTAACCGA(A/G)GACTGAGGGGAGAAGGATGT | 253 |
| cMOAT, ATP-binding cassette sub-family C member 2 | ABCC2 | PS101s30 | 35 | 650 | 1 | AGTTCAGGAATTAGATATTG(A/G)TTTACCACCACCCATGGC | 254 |
| cMOAT, ATP-binding cassette sub-family C member 2 | ABCC2 | PS101s31 | 35 | 742 | 1 | TCTAGTTTCCTCAAAGAGA(C/G)ACAGAACCCAGAAAGCAGAG | 255 |
| cMOAT, ATP-binding cassette sub-family C member 2 | ABCC2 | PS101s32 | 36 | 154 | 1 | GCAAGATGATGAAGAATAT(C/T)GAAAACAATCCTCTTGCTTG | 256 |
| cMOAT, ATP-binding cassette sub-family C member 2 | ABCC2 | PS101s33 | 36 | 680 | 1 | GGACATAATAATTCCTCCCT(A/G)TCAGAAAGATCCTCAGTATA | 257 |

| | REFSEQ | | | | | | | | | | | |
|---|---|---|---|---|---|---|---|---|---|---|---|---|
| | ALT (SEQ | | REF_SEQ_{—} | | | | MUTATION_{—} | REV_{—} | REF_{—} | ALT_{—} | CDNA_{—} | CDNA_{—} |
| GENE_DESCRIPTION | ID NO) | REF_SEQ_ID | POS | REF_NT | ALT_NT | EXON | TYPE | COMP | CODON | CODON | SEQ_ID | SEQ_POS |
| OATP2, solute carrier family 21 member 6 | 258 | AC022335 8 | 82621 | C | T | Exon5 | Silent | 1 | TCG | TCA | AF205071 1 | 545 |
| OATP2, solute carrier family 21 member 6 | 259 | AC022335 8 | 82589 | G | T | Exon5 | Missense | 1 | CCT | ACT | AF205071 1 | 597 |
| OATP2, solute carrier family 21 member 6 | 260 | AC022335 8 | 82391 | G | A | Intron5 | Non-CDS | 1 | | | AF205071 1 | |
| OATP2, solute carrier family 21 member 6 | 261 | AC022335 8 | 82386 | T | A | Intron5 | Non-CDS | 1 | | | AF205071 1 | |
| OATP2, solute carrier family 21 member 6 | 262 | AC022335 8 | 82362 | C | T | Intron5 | Non-CDS | 1 | | | AF205071 1 | |
| OATP2, solute carrier family 21 member 6 | 263 | AC022335 8 | 82360 | C | T | Intron5 | Non-CDS | 1 | | | AF205071 1 | |
| OATP2, solute carrier family 21 member 6 | 264 | AC022335 8 | 20092 | A | G | Exon5 | Non-CDS | 1 | | | AF205071 1 | 2377 |
| OATP2, solute carrier family 21 member 6 | 265 | AC022335 8 | 82550 | C | A | Intron5 | Non-CDS | 1 | | | AF205071 1 | |
| OATP2, solute carrier family 21 member 6 | 266 | AC022335 8 | 82644 | T | C | Exon5 | Missense | 1 | GAT | TAT | AF205071 1 | 522 |
| OATP2, solute carrier family 21 member 6 | 267 | AC022335 8 | 128151 | A | T | Intron1 | Non-CDS | 1 | | | AF205071 1 | |
| OATP2, solute carrier family 21 member 6 | 268 | AC022335 8 | 128152 | T | C | Intron1 | Non-CDS | 1 | | | AF205071 1 | |
| OATP2, solute carrier family 21 member 6 | 269 | AC022335 8 | 34576 | A | G | Intron14 | Non-CDS | 1 | | | AF205071 1 | |
| OATP2, solute carrier family 21 member 6 | 270 | AC022335 8 | 117572 | T | C | Intron2 | Non-CDS | 1 | | | AF205071 1 | |
| OATP2, solute carrier family 21 member 6 | 271 | AC022335 8 | 117597 | C | T | Intron2 | Non-CDS | 1 | | | AF205071 1 | |
| OATP2, solute carrier family 21 member 6 | 272 | AC022335 8 | 117587 | T | A | Intron2 | Non-CDS | 1 | | | AF205071 1 | |
| OATP2, solute carrier family 21 member 6 | 273 | AC022335 8 | 117583 | A | G | Intron2 | Non-CDS | 1 | | | AF205071 1 | |
| OATP2, solute carrier family 21 member 6 | 274 | AC022335 8 | 117582 | A | T | Intron2 | Non-CDS | 1 | | | AF205071 1 | |
| OATP2, solute carrier family 21 member 6 | 275 | AC022335 8 | 117542 | T | C | Intron2 | Non-CDS | 1 | | | AF205071 1 | |
| OATP2, solute carrier family 21 member 6 | 276 | AC022335 8 | 88574 | C | T | Intron3 | Non-CDS | 1 | | | AF205071 1 | |
| OATP2, solute carrier family 21 member 6 | 277 | AC022335 8 | 86568 | A | C | Intron3 | Non-CDS | 1 | | | AF205071 1 | |
| OATP2, solute carrier family 21 member 6 | 278 | AC022335 8 | 86433 | T | C | Intron3 | Non-CDS | 1 | | | AF205071 1 | |
| OATP2, solute carrier family 21 member 6 | 279 | AC022335 8 | 86404 | A | G | Intron3 | Non-CDS | 1 | | | AF205071 1 | |
| OATP2, solute carrier family 21 member 6 | 280 | AC022335 8 | 53449 | C | G | Exon11 | Missense | 1 | GGT | GCT | AF205071 1 | 1597 |
| OATP2, solute carrier family 21 member 6 | 281 | AC022335 8 | 53599 | C | G | Intron10 | Non-CDS | 1 | | | AF205071 1 | |
| OATP2, solute carrier family 21 member 6 | 282 | AC022335 8 | 56645 | C | T | Exon10 | Silent | 1 | GTG | GTA | AF205071 1 | 1382 |
| OATP2, solute carrier family 21 member 6 | 283 | AC022335 8 | 58893 | G | C | Exon10 | Missense | 1 | TTC | TTG | AF205071 1 | 1334 |
| OATP2, solute carrier family 21 member 6 | 284 | AC022335 8 | 57092 | A | G | Intron9 | Non-CDS | 1 | | | AF205071 1 | |
| OATP2, solute carrier family 21 member 6 | 285 | AC022335 8 | 42107 | C | G | Intron12 | Non-CDS | 1 | | | AF205071 1 | |
| OATP2, solute carrier family 21 member 6 | 286 | AC022335 8 | 60633 | A | G | Exon6 | Missense | 1 | GTG | GCG | AF205071 1 | 655 |
| OATP2, solute carrier family 21 member 6 | 287 | AC022335 8 | 60783 | A | G | Exon6 | Silent | 1 | TTG | CTG | AF205071 1 | 705 |
| OATP2, solute carrier family 21 member 6 | 288 | AC022335 8 | 60757 | G | A | Exon6 | Silent | 1 | TTC | TTT | AF205071 1 | 731 |
| OATP2, solute carrier family 21 member 6 | 289 | AC022335 8 | 80385 | G | A | Intron7 | Non-CDS | 1 | | | AF205071 1 | |
| OATP2, solute carrier family 21 member 6 | 290 | AC022335 8 | 19796 | T | C | Exon15 | Non-CDS | 1 | | | AF205071 1 | 2673 |
| OATP2, solute carrier family 21 member 6 | 291 | AC022335 8 | 19810 | T | G | Exon15 | Non-CDS | 1 | | | AF205071 1 | 2658 |
| OATP2, solute carrier family 21 member 6 | 292 | AC022335 8 | 19820 | A | C | Exon15 | Non-CDS | 1 | | | AF205071 1 | 2649 |
| OATP2, solute carrier family 21 member 6 | 293 | AC022335 8 | 19831 | T | C | Exon15 | Non-CDS | 1 | | | AF205071 1 | 2538 |
| cMOAT, ATP-binding cassette sub-family C member 2 | 294 | AL392107 4 | 56274 | A | T | Exon25 | Missense | 1 | GAG | GTG | U49248 1 | 3664 |
| cMOAT, ATP-binding cassette sub-family C member 2 | 295 | AL392107 4 | 58303 | G | A | Exon28 | Silent | 1 | ATC | ATT | U49248 1 | 4073 |
| cMOAT, ATP-binding cassette sub-family C member 2 | 296 | AL392107 4 | 56960 | C | G | Intron29 | Non-CDS | 1 | | | U49248 1 | |
| cMOAT, ATP-binding cassette sub-family C member 2 | 297 | AL392107 4 | 57007 | G | A | Exon29 | Silent | 1 | CTC | CTT | U49248 1 | 4211 |
| cMOAT, ATP-binding cassette sub-family C member 2 | 298 | AL392107 4 | 57055 | G | A | Exon29 | Silent | 1 | GCC | GCT | U49248 1 | 4163 |
| cMOAT, ATP-binding cassette sub-family C member 2 | 299 | AL392107 4 | 52055 | C | T | Exon31 | Silent | 1 | GAG | GAA | U49248 1 | 4511 |
| cMOAT, ATP-binding cassette sub-family C member 2 | 300 | AL392107 4 | 51977 | G | A | Exon31 | Silent | 1 | CAT | CAC | U49248 1 | 4589 |
| cMOAT, ATP-binding cassette sub-family C member 2 | 301 | AL392107 4 | 51945 | C | T | Intron31 | Non-CDS | 1 | | | U49248 1 | |
| cMOAT, ATP-binding cassette sub-family C member 2 | 302 | AL392107 4 | 51883 | G | A | Intron31 | Non-CDS | 1 | | | U49248 1 | |
| cMOAT, ATP-binding cassette sub-family C member 2 | 303 | AL392107 4 | 66295 | C | A | Exon25 | Missense | 1 | CGA | CTA | U49248 1 | 3643 |
| cMOAT, ATP-binding cassette sub-family C member 2 | 304 | AL392107 4 | 71683 | T | C | Exon21 | Missense | 1 | AAG | AGG | U49248 1 | 2963 |
| cMOAT, ATP-binding cassette sub-family C member 2 | 305 | AL392107 4 | 71651 | G | A | Intron21 | Non-CDS | 1 | | | U49248 1 | |
| cMOAT, ATP-binding cassette sub-family C member 2 | 306 | AL392107 4 | 110259 | 1 | C | Exon3 | Silent | 1 | GTA | GTG | U49248 1 | 359 |
| cMOAT, ATP-binding cassette sub-family C member 2 | 307 | AL392107 4 | 94484 | T | A | Intron12 | Non-CDS | 1 | | | U49248 1 | |
| cMOAT, ATP-binding cassette sub-family C member 2 | 308 | AL392107 4 | 90742 | G | A | Intron15 | Non-CDS | 1 | | | U49248 1 | |
| cMOAT, ATP-binding cassette sub-family C member 2 | 309 | AL392107 4 | 90828 | C | A | Intron15 | Non-CDS | 1 | | | U49248 1 | |
| cMOAT, ATP-binding cassette sub-family C member 2 | 310 | AL392107 4 | 72250 | C | T | Intron19 | Non-CDS | 1 | | | U49248 1 | |
| cMOAT, ATP-binding cassette sub-family C member 2 | 311 | AL392107 4 | 72272 | A | G | Intron19 | Non-CDS | 1 | | | U49248 1 | |
| cMOAT, ATP-binding cassette sub-family C member 2 | 312 | AL392107 4 | 119722 | G | A | Exon1 | Non-CDS | 1 | | | U49248 1 | 78 |
| cMOAT, ATP-binding cassette sub-family C member 2 | 313 | AL392107 4 | 58988 | A | G | Intron25 | Non-CDS | 1 | | | U49248 1 | |
| cMOAT, ATP-binding cassette sub-family C member 2 | 314 | AL392107 4 | 94844 | C | T | Intron12 | Non-CDS | 1 | | | U49248 1 | |
| cMOAT, ATP-binding cassette sub-family C member 2 | 315 | AL392107 4 | 89454 | A | G | Exon16 | Missense | 1 | ATA | ACA | U49248 1 | 2110 |
| cMOAT, ATP-binding cassette sub-family C member 2 | 316 | AL392107 4 | 98465 | C | T | Exon10 | Missense | 1 | GTT | ATT | U49248 1 | 1350 |
| cMOAT, ATP-binding cassette sub-family C member 2 | 317 | AL392107 4 | 98496 | G | A | Exon10 | Missense | 1 | CTA | TTA | U49248 1 | 1320 |
| cMOAT, ATP-binding cassette sub-family C member 2 | 318 | AL392107 4 | 105455 | C | A | Intron6 | Non-CDS | 1 | | | U49248 1 | |
| cMOAT, ATP-binding cassette sub-family C member 2 | 319 | AL392107 4 | 57834 | A | G | Intron24 | Non-CDS | 1 | | | U49248 1 | |
| cMOAT, ATP-binding cassette sub-family C member 2 | 320 | AL392107 4 | 57861 | C | A | Intron24 | Non-CDS | 1 | | | U49248 1 | |
| cMOAT, ATP-binding cassette sub-family C member 2 | 321 | AL392107 4 | 57870 | G | A | Intron24 | Non-CDS | 1 | | | U49248 1 | |
| cMOAT, ATP-binding cassette sub-family C member 2 | 322 | AL392107 4 | 109039 | A | G | Intron3 | Non-CDS | 1 | | | U49248 1 | |
| cMOAT, ATP-binding cassette sub-family C member 2 | 323 | AL392107 4 | 83188 | A | G | Intron19 | Non-CDS | 1 | | | U49248 1 | |
| cMOAT, ATP-binding cassette sub-family C member 2 | 324 | AL392107 4 | 83096 | C | G | Intron19 | Non-CDS | 1 | | | U49248 1 | |
| cMOAT, ATP-binding cassette sub-family C member 2 | 325 | AL392107 4 | 70852 | C | T | Exon22 | Silent | 1 | TCG | TCA | U49248 1 | 3025 |
| cMOAT, ATP-binding cassette sub-family C member 2 | 326 | AL392107 4 | 70326 | A | G | Intron23 | Non-CDS | 1 | | | U49248 1 | |

TABLE VI

| | | | | CONTIG_{—} | REF_{—} | ALT_{—} | | MUTATION_{—} |
|---|---|---|---|---|---|---|---|---|
| GENE_DESCRIPTION | HGNC_ID | SNP_ID | CONTIG_NUM | POS | AA | AA | EXON | TYPE |
| OATP2, solute carrier family 21 member 6 | SLC21A6 | PS100s1 | 4 | 208 | S | S | Exon5 | Silent |
| OATP2, solute carrier family 21 member 6 | SLC21A6 | PS100s2 | 4 | 260 | P | T | Exon5 | Missense |
| OATP2, solute carrier family 21 member 6 | SLC21A6 | PS100s9 | 9 | 308 | D | Y | Exon5 | Missense |
| OATP2, solute carrier family 21 member 6 | SLC21A6 | PS100s23 | 16 | 244 | G | A | Exon11 | Missense |
| OATP2, solute carrier family 21 member 6 | SLC21A6 | PS100s25 | 17 | 133 | V | V | Exon10 | Silent |
| OATP2, solute carrier family 21 member 6 | SLC21A6 | PS100s26 | 17 | 181 | F | K | Exon10 | Missense |
| OATP2, solute carrier family 21 member 6 | SLC21A6 | PS100s29 | 19 | 75 | V | A | Exon6 | Missense |
| OATP2, solute carrier family 21 member 6 | SLC21A6 | PS100s30 | 19 | 125 | L | L | Exon6 | Silent |
| OATP2, solute carrier family 21 member 6 | SLC21A6 | PS100s31 | 19 | 151 | F | F | Exon6 | Silent |
| cMOAT, ATP-binding cassette sub-family C | ABCC2 | PS101s1 | 4 | 284 | E | V | Exon25 | Missense |
| member 2 | | | | | | | | |
| cMOAT, ATP-binding cassette sub-family C | ABCC2 | PS101s2 | 6 | 213 | I | I | Exon28 | Silent |
| member 2 | | | | | | | | |
| cMOAT, ATP-binding cassette sub-family C | ABCC2 | PS101s4 | 7 | 100 | L | L | Exon29 | Silent |
| member 2 | | | | | | | | |
| cMOAT, ATP-binding cassette sub-family C | ABCC2 | PS101s5 | 7 | 148 | A | A | Exon29 | Silent |
| member 2 | | | | | | | | |
| cMOAT, ATP-binding cassette sub-family C | ABCC2 | PS101s6 | 9 | 112 | E | E | Exon31 | Silent |
| member 2 | | | | | | | | |
| cMOAT, ATP-binding cassette sub-family C | ABCC2 | PS101s7 | 9 | 190 | H | H | Exon31 | Silent |
| member 2 | | | | | | | | |
| cMOAT, ATP-binding cassette sub-family C | ABCC2 | PS101s10 | 13 | 264 | R | L | Exon25 | Missense |
| member 2 | | | | | | | | |
| cMOAT, ATP-binding cassette sub-family C | ABCC2 | PS101s11 | 15 | 192 | K | R | Exon21 | Missense |
| member 2 | | | | | | | | |
| cMOAT, ATP-binding cassette sub-family C | ABCC2 | PS101s13 | 16 | 859 | V | V | Exon3 | Slient |
| member 2 | | | | | | | | |
| cMOAT, ATP-binding cassette sub-family C | ABCC2 | PS101s22 | 29 | 136 | I | T | Exon16 | Missense |
| member 2 | | | | | | | | |
| cMOAT, ATP-binding cassette sub-family C | ABCC2 | PS101s23 | 30 | 545 | V | I | Exon10 | Missense |
| member 2 | | | | | | | | |
| cMOAT, ATP-binding cassette sub-family C | ABCC2 | PS101s24 | 30 | 575 | L | K | Exon10 | Missense |
| member 2 | | | | | | | | |
| cMOAT, ATP-binding cassette sub-family C | ABCC2 | PS101s32 | 36 | 164 | S | S | Exon22 | Silent |
| member 2 | | | | | | | | |

| | | | | | | | REFSEQ | REFSEQ |
|---|---|---|---|---|---|---|---|---|
| | | | | | | | REF | ALT |
| | REV_{—} | REF_{—} | ALT_{—} | | PROTEIN_{—} | PROTEIN | (SEQ ID | (SEQ ID |
| GENE_DESCRIPTION | COMP | CODON | CODON | PROTEIN_ID | POS | (SEQ ID NO:) | NO:) | NO:) |
| OATP2, solute carrier family 21 member 6 | 1 | TCG | TCA | AAF20212_1 | 137 | 6 | 189 | 258 |
| OATP2, solute carrier family 21 member 6 | 1 | CCT | ACT | AAF20212_1 | 155 | 8 | 190 | 259 |
| OATP2, solute carrier family 21 member 6 | 1 | GAT | TAT | AAF20212_1 | 130 | 10 | 197 | 266 |
| OATP2, solute carrier family 21 member 6 | 1 | GGT | GCT | AAF20212_1 | 488 | 12 | 270 | 270 |
| OATP2, solute carrier family 21 member 6 | 1 | GTG | GTA | AAF20212_1 | 416 | 14 | 213 | 282 |
| OATP2, solute carrier family 21 member 6 | 1 | TTC | TTG | AAF20212_1 | 400 | 16 | 214 | 283 |
| OATP2, solute carrier family 21 member 6 | 1 | GTG | GCG | AAF20212_1 | 174 | 18 | 217 | 286 |
| OATP2, solute carrier family 21 member 6 | 1 | TTG | CTG | AAF20212_1 | 191 | 20 | 218 | 287 |
| OATP2, solute carrier family 21 member 6 | 1 | TTC | TTT | AAF20212_1 | 199 | 22 | 219 | 288 |
| cMOAT, ATP-binding cassette sub-family C | 1 | GAG | GTG | AAB09422_1 | 1,188 | 24 | 225 | 294 |
| member 2 | | | | | | | | |
| cMOAT, ATP-binding cassette sub-family C | 1 | ATC | ATT | AAB09422_1 | 1,324 | 26 | 226 | 295 |
| member 2 | | | | | | | | |
| cMOAT, ATP-binding cassette sub-family C | 1 | CTC | CTT | AAB09422_1 | 1,370 | 28 | 228 | 297 |
| member 2 | | | | | | | | |
| cMOAT, ATP-binding cassette sub-family C | 1 | GCC | GCT | AAB09422_1 | 1,354 | 30 | 229 | 298 |
| member 2 | | | | | | | | |
| cMOAT, ATP-binding cassette sub-family C | 1 | GAG | GAA | AAB09422_1 | 1,470 | 32 | 230 | 299 |
| member 2 | | | | | | | | |
| cMOAT, ATP-binding cassette sub-family C | 1 | CAT | CAC | AAB09422_1 | 1,496 | 34 | 231 | 300 |
| member 2 | | | | | | | | |
| cMOAT, ATP-binding cassette sub-family C | 1 | CGA | CTA | AAB09422_1 | 1,181 | 36 | 234 | 303 |
| member 2 | | | | | | | | |
| cMOAT, ATP-binding cassette sub-family C | 1 | AAG | AGG | AAB09422_1 | 961 | 38 | 235 | 304 |
| member 2 | | | | | | | | |
| cMOAT, ATP-binding cassette sub-family C | 1 | GTA | GTG | AAB09422_1 | 86 | 40 | 237 | 306 |
| member 2 | | | | | | | | |
| cMOAT, ATP-binding cassette sub-family C | 1 | ATA | ACA | AAB09422_1 | 670 | 42 | 246 | 315 |
| member 2 | | | | | | | | |
| cMOAT, ATP-binding cassette sub-family C | 1 | GTT | ATT | AAB09422_1 | 417 | 44 | 247 | 316 |
| member 2 | | | | | | | | |
| cMOAT, ATP-binding cassette sub-family C | 1 | CTA | TTA | AAB06422_1 | 407 | 46 | 248 | 317 |
| member 2 | | | | | | | | |
| cMOAT, ATP-binding cassette sub-family C | 1 | TCG | TCA | AAB09422_1 | 978 | 48 | 256 | 325 |
| member 2 | | | | | | | | |

TABLE VII

| Ethnicity | Coriell DNA Sample ID | Plate No. |
|---|---|---|
| African American | NA14905 | 1 |
| African American | NA14922 | 2 |
| African American | NA14923 | 3 |
| African American | NA14924 | 4 |
| African American | NA14925 | 5 |
| African American | NA14932 | 6 |
| African American | NA14933 | 7 |
| African American | NA14934 | 8 |
| Caucasian | NA 17201 | 9 |
| Caucasian | NA17202 | 10 |
| Caucasian | NA17203 | 11 |
| Caucasian | NA17204 | 12 |
| Caucasian | NA17205 | 13 |
| Caucasian | NA17206 | 14 |
| Caucasian | NA17207 | 15 |
| Caucasian | NA17208 | 16 |
| Chinese | NA00576 | 17 |
| Chinese | NA03433 | 18 |
| Chinese | NA06090 | 19 |
| Chinese | NA07426 | 20 |
| Japaness | NA02345b | 21 |
| Japaness | NA11589 | 22 |
| Japaness | NA14819 | 23 |
| Japaness | NA04535 | 24 |

TABLE VIII

| | | | | | PCR |
|---|---|---|---|---|---|
| | | | | | Amplicon_{—} |
| GENE_DESCRIPTION | HGNC_ID | SNP_ID | EXON | REV_COMP | Name |
| OATP2, solute carrier family 21 member 6 | SLC21A6 | PS100s1 | Exon5 | 1 | PS100p49p50 |
| OATP2, solute carrier family 21 member 6 | SLC21A6 | PS100s2 | Exon5 | 1 | PS100p49p50 |
| OATP2, solute carrier family 21 member 6 | SLC21A6 | PS100s3 | Intron5 | 1 | PS100p49p50 |
| OATP2, solute carrier family 21 member 6 | SLC21A6 | PS100s4 | Intron5 | 1 | PS100p49p50 |
| OATP2, solute carrier family 21 member 6 | SLC21A6 | PS100s5 | Intron5 | 1 | PS100p49p50 |
| OATP2, solute carrier family 21 member 6 | SLC21A6 | PS100s6 | Intron5 | 1 | PS100p49p50 |
| OATP2, solute carrier family 21 member 6 | SLC21A6 | PS100s7 | Exon15 | 1 | PS100p5p6 |
| OATP2, solute carrier family 21 member 6 | SLC21A6 | PS100s8 | Intron15 | 1 | PS100p49p50 |
| OATP2, solute carrier family 21 member 6 | SLC21A6 | PS100s9 | Exon5 | 1 | PS100p49p50 |
| OATP2, solute carrier family 21 member 6 | SLC21A6 | PS100s10 | Intron1 | 1 | PS100p65p66 |
| OATP2, solute carrier family 21 member 6 | SLC21A6 | PS100s11 | Intron1 | 1 | PS100p65p66 |
| OATP2, solute carrier family 21 member 6 | SLC21A6 | PS100s12 | Intron14 | 1 | PS100p13p14 |
| OATP2, solute carrier family 21 member 6 | SLC21A6 | PS100s13 | Intron2 | 1 | PS100p61p62 |
| OATP2, solute carrier family 21 member 6 | SLC21A6 | PS100s14 | Intron2 | 1 | PS100p61p62 |
| OATP2, solute carrier family 21 member 6 | SLC21A6 | PS100s15 | Intron2 | 1 | PS100p61p62 |
| OATP2, solute carrier family 21 member 6 | SLC21A6 | PS100s16 | Intron2 | 1 | PS100p61p62 |
| OATP2, solute carrier family 21 member 6 | SLC21A6 | PS100s17 | Intron2 | 1 | PS100p61p62 |
| OATP2, solute carrier family 21 member 6 | SLC21A6 | PS100s18 | Intron2 | 1 | PS100p61p62 |
| OATP2, solute carrier family 21 member 6 | SLC21A6 | PS100s19 | Intron3 | 1 | PS100p57p58 |
| OATP2, solute carrier family 21 member 6 | SLC21A6 | PS100s20 | Intron3 | 1 | PS100p57p58 |
| OATP2, solute carrier family 21 member 6 | SLC21A6 | PS100s21 | Intron3 | 1 | PS100p57p58 |
| OATP2, solute carrier family 21 member 6 | SLC21A6 | PS100s22 | Intron3 | 1 | PS100p57p58 |
| OATP2, solute carrier family 21 member 6 | SLC21A6 | PS100s23 | Exon11 | 1 | PS100p25p26 |
| OATP2, solute carrier family 21 member 6 | SLC21A6 | PS100s24 | Intron10 | 1 | PS100p25p26 |
| OATP2, solute carrier family 21 member 6 | SLC21A6 | PS100s25 | Exon10 | 1 | PS100p31p32 |
| OATP2, solute carrier family 21 member 6 | SLC21A6 | PS100s26 | Exon10 | 1 | PS100p31p32 |
| OATP2, solute carrier family 21 member 6 | SLC21A6 | PS100s27 | Intron9 | 1 | PS100p31p32 |
| OATP2, solute carrier family 21 member 6 | SLC21A6 | PS100s28 | Intron12 | 1 | PS100p21p22 |
| OATP2, solute carrier family 21 member 6 | SLC21A6 | PS100s29 | Exon6 | 1 | PS100p45p46 |
| OATP2, solute carrier family 21 member 6 | SLC21A6 | PS100s30 | Exon6 | 1 | PS100p45p46 |
| OATP2, solute carrier family 21 member 6 | SLC21A6 | PS100s31 | Exon6 | 1 | PS100p45p46 |
| OATP2, solute carrier family 21 member 6 | SLC21A6 | PS100s32 | Intron7 | 1 | PS100p41p42 |
| OATP2, solute carrier family 21 member 6 | SLC21A6 | PS100s34 | Exon15 | 1 | PS100p1p2 |
| OATP2, solute carrier family 21 member 6 | SLC21A6 | PS100s35 | Exon15 | 1 | PS100p1p2 |
| OATP2, solute carrier family 21 member 6 | SLC21A6 | PS100s36 | Exon15 | 1 | PS100p5p6 |
| cMOAT, ATP-binding cassette sub-family C member 2 | ABCC2 | PS101s1 | Exon25 | 1 | PS101p33p34 |
| cMOAT, ATP-binding cassette sub-family C member 2 | ABCC2 | PS101s2 | Exon28 | 1 | PS101p21p22 |
| cMOAT, ATP-binding cassette sub-family C member 2 | ABCC2 | PS101s3 | Intron29 | 1 | PS101p17p18 |
| cMOAT, ATP-binding cassette sub-family C member 2 | ABCC2 | PS101s4 | Exon29 | 1 | PS101p17p18 |
| cMOAT, ATP-binding cassette sub-family C member 2 | ABCC2 | PS101s5 | Exon29 | 1 | PS101p17p18 |
| cMOAT, ATP-binding cassette sub-family C member 2 | ABCC2 | PS101s6 | Exon31 | 1 | PS101p9p10 |
| cMOAT, ATP-binding cassette sub-family C member 2 | ABCC2 | PS101s7 | Exon31 | 1 | PS101p9p10 |
| cMOAT, ATP-binding cassette sub-family C member 2 | ABCC2 | PS101s8 | Intron31 | 1 | PS101p9p10 |
| cMOAT, ATP-binding cassette sub-family C member 2 | ABCC2 | PS101s9 | Intron31 | 1 | PS101p9p10 |
| cMOAT, ATP-binding cassette sub-family C member 2 | ABCC2 | PS101s10 | Exon25 | 1 | PS101p33p34 |
| cMOAT, ATP-binding cassette sub-family C member 2 | ABCC2 | PS101s11 | Exon21 | 1 | PS101p49p50 |
| cMOAT, ATP-binding cassette sub-family C member 2 | ABCC2 | PS101s12 | Intron21 | 1 | PS101p49p50 |
| cMOAT, ATP-binding cassette sub-family C member 2 | ABCC2 | PS101s13 | Exon3 | 1 | PS101p121p122 |
| cMOAT, ATP-binding cassette sub-family C member 2 | ABCC2 | PS101s14 | Intron12 | 1 | PS101p81p82 |
| cMOAT, ATP-binding cassette sub-family C member 2 | ABCC2 | PS101s15 | Intron15 | 1 | PS101p73p74 |
| cMOAT, ATP-binding cassette sub-family C member 2 | ABCC2 | PS101s16 | Intron15 | 1 | PS101p73p74 |
| cMOAT, ATP-binding cassette sub-family C member 2 | ABCC2 | PS101s17 | Intron19 | 1 | PS101p53p54 |
| cMOAT, ATP-binding cassette sub-family C member 2 | ABCC2 | PS101s18 | Intron19 | 1 | PS101p53p54 |
| cMOAT, ATP-binding cassette sub-family C member 2 | ABCC2 | PS101s19 | Exon1 | 1 | PS101p129p130 |
| cMOAT, ATP-binding cassette sub-family C member 2 | ABCC2 | PS101s20 | Intron26 | 1 | PS101p25p26 |
| cMOAT, ATP-binding cassette sub-family C member 2 | ABCC2 | PS101s21 | Intron12 | 1 | PS101p85p86 |
| cMOAT, ATP-binding cassette sub-family C member 2 | ABCC2 | PS101s22 | Exon16 | 1 | PS101p69p70 |
| cMOAT, ATP-binding cassette sub-family C member 2 | ABCC2 | PS101s23 | Exon10 | 1 | PS101p93p94 |
| cMOAT, ATP-binding cassette sub-family C member 2 | ABCC2 | PS101s24 | Exon10 | 1 | PS101p93p94 |
| cMOAT, ATP-binding cassette sub-family C member 2 | ABCC2 | PS101s25 | Intron6 | 1 | PS101p105p106 |
| cMOAT, ATP-binding cassette sub-family C member 2 | ABCC2 | PS101s26 | Intron24 | 1 | PS101p37p38 |
| cMOAT, ATP-binding cassette sub-family C member 2 | ABCC2 | PS101s27 | Intron24 | 1 | PS101p37p38 |
| cMOAT, ATP-binding cassette sub-family C member 2 | ABCC2 | PS101s28 | Intron24 | 1 | PS101p37p38 |
| cMOAT, ATP-binding cassette sub-family C member 2 | ABCC2 | PS101s29 | Intron3 | 1 | PS101p117p118 |
| cMOAT, ATP-binding cassette sub-family C member 2 | ABCC2 | PS101s30 | Intron19 | 1 | PS101p57p58 |
| cMOAT, ATP-binding cassette sub-family C member 2 | ABCC2 | PS101s31 | Intron19 | 1 | PS101p57p58 |
| cMOAT, ATP-binding cassette sub-family C member 2 | ABCC2 | PS101s32 | Exon22 | 1 | PS101p45p46 |
| cMOAT, ATP-binding cassette sub-family C member 2 | ABCC2 | PS101s33 | Intron23 | 1 | PS101p41p42 |

| GENE_DESCRIPTION | Target_Name | PCR Left primer |
|---|---|---|
| OATP2, solute carrier family 21 member 6 | SLC21A6_X5a | TTCAGATGGACAAAATTTGCA |
| OATP2, solute carrier family 21 member 6 | SLC21A6_X5a | TTCAGATGGACAAAATTTGCA |
| OATP2, solute carrier family 21 member 6 | SLC21A6_X5a | TTCAGATGGACAAAATTTGCA |
| OATP2, solute carrier family 21 member 6 | SLC21A6_X5a | TTCAGATGGACAAAATTTGCA |
| OATP2, solute carrier family 21 member 6 | SLC21A6_X5a | TTCAGATGGACAAAATTTGCA |
| OATP2, solute carrier family 21 member 6 | SLC21A6_X5a | TTCAGATGGACAAAATTTGCA |
| OATP2, solute carrier family 21 member 6 | SLC21A6_X15_f2a | CCAAGCTAGACTTCAGGCCTT |
| OATP2, solute carrier family 21 member 6 | SLC21A6_X5a | TTCAGATGGACAAAATTTGCA |
| OATP2, solute carrier family 21 member 6 | SLC21A6_X5a | TTCAGATGGACAAAATTTGCA |
| OATP2, solute carrier family 21 member 6 | SLC21A6_X1a | GGCTCTTCTACTCCCAGAAGG |
| OATP2, solute carrier family 21 member 6 | SLC21A6_X1a | GGCTCTTCTACTCCCAGAAGG |
| OATP2, solute carrier family 21 member 6 | SLC21A6_X14a | AAAATGAGATACGAGATTGCTTGA |
| OATP2, solute carrier family 21 member 6 | SLC21A6_X2a | CGTGATCAATCCAAAACCAAA |
| OATP2, solute carrier family 21 member 6 | SLC21A6_X2a | CGTGATCAATCCAAAACCAAA |
| OATP2, solute carrier family 21 member 6 | SLC21A6_X2a | CGTGATCAATCCAAAACCAAA |
| OATP2, solute carrier family 21 member 6 | SLC21A6_X2a | CGTGATCAATCCAAAACCAAA |
| OATP2, solute carrier family 21 member 6 | SLC21A6_X2a | CGTGATCAATCCAAAACCAAA |
| OATP2, solute carrier family 21 member 6 | SLC21A6_X2a | CGTGATCAATCCAAAACCAAA |
| OATP2, solute carrier family 21 member 6 | SLC21A6_X3a | CAGGGTTTCTGAACATCCTCA |
| OATP2, solute carrier family 21 member 6 | SLC21A6_X3a | CAGGGTTTCTGAACATCCTCA |
| OATP2, solute carrier family 21 member 6 | SLC21A6_X3a | CAGGGTTTCTGAACATCCTCA |
| OATP2, solute carrier family 21 member 6 | SLC21A6_X3a | CAGGGTTTCTGAACATCCTCA |
| OATP2, solute carrier family 21 member 6 | SLC21A6_X11a | AGAAAAACCTGATTGTGCCCT |
| OATP2, solute carrier family 21 member 6 | SLC21A6_X11a | AGAAAAACCTGATTGTGCCCT |
| OATP2, solute carrier family 21 member 6 | SLC21A6_X10 | TTTGATGGTTAACATATTATGCAATT |
| OATP2, solute carrier family 21 member 6 | SLC21A6_X10 | TTTGATGGTTAACATATTATGCAATT |
| OATP2, solute carrier family 21 member 6 | SLC21A6_X10 | TTTGATGGTTAACATATTATGCAATT |
| OATP2, solute carrier family 21 member 6 | SLC21A6_X12a | CAGCCTTGAGAGTTCATAGTAATTTT |
| OATP2, solute carrier family 21 member 6 | SLC21A6_X6a | TTTTAGAAAACAGAGATCCCAGG |
| OATP2, solute carrier family 21 member 6 | SLC21A6_X6a | TTTTAGAAAACAGAGATCCCAGG |
| OATP2, solute carrier family 21 member 6 | SLC21A6_X6a | TTTTAGAAAACAGAGATCCCAGG |
| OATP2, solute carrier family 21 member 6 | SLC21A6_X7a | GCAAAGGACTATTGAAAGAGTGAA |
| OATP2, solute carrier family 21 member 6 | SLC21A6_X15_f1a | TTTTTCCTAATACATTACCGTGG |
| OATP2, solute carrier family 21 member 6 | SLC21A6_X15_f1a | TTTTTCCTAATACATTACCGTGG |
| OATP2, solute carrier family 21 member 6 | SLC21A6_X15_f2a | CCAAGCTAGACTTCAGGCCTT |
| cMOAT, ATP-binding cassette sub-family C member 2 | ABCC2_X25a | TAGCATGACAGTGTGTGTGGC |
| cMOAT, ATP-binding cassette sub-family C member 2 | ABCC2_X28a | AGGGAGGAGCTGAGCAGAAG |
| cMOAT, ATP-binding cassette sub-family C member 2 | ABCC2_X29a | ACGTGTCCATCCTAGGGAACT |
| cMOAT, ATP-binding cassette sub-family C member 2 | ABCC2_X29a | ACGTGTCCATCCTAGGGAACT |
| cMOAT, ATP-binding cassette sub-family C member 2 | ABCC2_X29a | ACGTGTCCATCCTAGGGAACT |
| cMOAT, ATP-binding cassette sub-family C member 2 | ABCC2_X31a | TCATCAGTAATGTGGTGGTGG |
| cMOAT, ATP-binding cassette sub-family C member 2 | ABCC2_X31a | TCATCAGTAATGTGGTGGTGG |
| cMOAT, ATP-binding cassette sub-family C member 2 | ABCC2_X31a | TCATCAGTAATGTGGTGGTGG |
| cMOAT, ATP-binding cassette sub-family C member 2 | ABCC2_X31a | TCATCAGTAATGTGGTGGTGG |
| cMOAT, ATP-binding cassette sub-family C member 2 | ABCC2_X25a | TAGCATGACAGTGTGTGTGGC |
| cMOAT, ATP-binding cassette sub-family C member 2 | ABCC2_X21a | TTTCCTGTTCCTCCACACATC |
| cMOAT, ATP-binding cassette sub-family C member 2 | ABCC2_X21a | TTTCCTGTTCCTCCACACATC |
| cMOAT, ATP-binding cassette sub-family C member 2 | ABCC2_X3a | TGCTCAAATGAAGATGAAAAGC |
| cMOAT, ATP-binding cassette sub-family C member 2 | ABCC2_X13a | ACTGGGGCTCAGAAAACTCAT |
| cMOAT, ATP-binding cassette sub-family C member 2 | ABCC2_X15a | TGGGATCACAGCTGGATATTC |
| cMOAT, ATP-binding cassette sub-family C member 2 | ABCC2_X15a | TGGGATCACAGCTGGATATTC |
| cMOAT, ATP-binding cassette sub-family C member 2 | ABCC2_X20a | AGATGACCAGGGGAAACTGAC |
| cMOAT, ATP-binding cassette sub-family C member 2 | ABCC2_X20a | AGATGACCAGGGGAAACTGAC |
| cMOAT, ATP-binding cassette sub-family C member 2 | ABCC2_X1a | CCTGAGCTTTAGACCAATTGC |
| cMOAT, ATP-binding cassette sub-family C member 2 | ABCC2_X27a | GGGCCCATGTAAAATATGGAG |
| cMOAT, ATP-binding cassette sub-family C member 2 | ABCC2_X12a | GGGGTATGGTACAAAGGAGGA |
| cMOAT, ATP-binding cassette sub-family C member 2 | ABCC2_X16a | GGATGTCTCCAAGACCTCACC |
| cMOAT, ATP-binding cassette sub-family C member 2 | ABCC2_X10a | AAAGTCTTCCACCAGCTTTGC |
| cMOAT, ATP-binding cassette sub-family C member 2 | ABCC2_X10a | AAAGTCTTCCACCAGCTTTGC |
| cMOAT, ATP-binding cassette sub-family C member 2 | ABCC2_X7a | GTAAGGACAGAATCGCCATGA |
| cMOAT, ATP-binding cassette sub-family C member 2 | ABCC2_X24a | ACTTCAGCTTCAGACAGTGGC |
| cMOAT, ATP-binding cassette sub-family C member 2 | ABCC2_X24a | ACTTCAGCTTCAGACAGTGGC |
| cMOAT, ATP-binding cassette sub-family C member 2 | ABCC2_X24a | ACTTCAGCTTCAGACAGTGGC |
| cMOAT, ATP-binding cassette sub-family C member 2 | ABCC2_X4a | GAACAGGCAGGAGTAGGCTAGA |
| cMOAT, ATP-binding cassette sub-family C member 2 | ABCC2_X19a | GCCCAGGCATAGAGTTTCAAT |
| cMOAT, ATP-binding cassette sub-family C member 2 | ABCC2_X19a | GCCCAGGCATAGAGTTTCAAT |
| cMOAT, ATP-binding cassette sub-family C member 2 | ABCC2_X22a | GATGGAAGCAGTTTTGTCAGC |
| cMOAT, ATP-binding cassette sub-family C member 2 | ABCC2_X23a | GGAGCTCACAGCAGGTACTCA |

| | PCR Left | | PCR Right |
|---|---|---|---|
| | primer | | primer |
| | (SEQ | | (SEQ |
| GENE_DESCRIPTION | ID NO:) | PCR Right primer | ID NO:) |
| OATP2, solute carrier family 21 member 6 | 327 | AAAACACATGCTGGGAAATTG | 396 |
| OATP2, solute carrier family 21 member 6 | 328 | AAAACACATGCTGGGAAATTG | 397 |
| OATP2, solute carrier family 21 member 6 | 329 | AAAACACATGCTGGGAAATTG | 398 |
| OATP2, solute carrier family 21 member 6 | 330 | AAAACACATGCTGGGAAATTG | 399 |
| OATP2, solute carrier family 21 member 6 | 331 | AAAACACATGCTGGGAAATTG | 400 |
| OATP2, solute carrier family 21 member 6 | 332 | AAAACACATGCTGGGAAATTG | 401 |
| OATP2, solute carrier family 21 member 6 | 333 | CTGGGGCAGATAGTGAAACAC | 402 |
| OATP2, solute carrier family 21 member 6 | 334 | AAAACACATGCTGGGAAATTG | 403 |
| OATP2, solute carrier family 21 member 6 | 335 | AAAACACATGCTGGGAAATTG | 404 |
| OATP2, solute carrier family 21 member 6 | 336 | TGTGAGAATTCTGAGAAATATAATCTTTAA | 405 |
| OATP2, solute carrier family 21 member 6 | 337 | TGTGAGAATTCTGAGAAATATAATCTTTAA | 406 |
| OATP2, solute carrier family 21 member 6 | 338 | TTGGGTAGATGCAGAACAAAA | 407 |
| OATP2, solute carrier family 21 member 6 | 339 | TGATGATCTTGTGGCTTTTCTT | 408 |
| OATP2, solute carrier family 21 member 6 | 340 | TGATGATCTTGTGGCTTTTCTT | 409 |
| OATP2, solute carrier family 21 member 6 | 341 | TGATGATCTTGTGGCTTTTCTT | 410 |
| OATP2, solute carrier family 21 member 6 | 342 | TGATGATCTTGTGGCTTTTCTT | 411 |
| OATP2, solute carrier family 21 member 6 | 343 | TGATGATCTTGTGGCTTTTCTT | 412 |
| OATP2, solute carrier family 21 member 6 | 344 | TGATGATCTTGTGGCTTTTCTT | 413 |
| OATP2, solute carrier family 21 member 6 | 345 | CCCCCTTTCCTTCTGATTTTT | 414 |
| OATP2, solute carrier family 21 member 6 | 346 | CCCCCTTTCCTTCTGATTTTT | 415 |
| OATP2, solute carrier family 21 member 6 | 347 | CCCCCTTTCCTTCTGATTTTT | 416 |
| OATP2, solute carrier family 21 member 6 | 348 | CCCCCTTTCCTTCTGATTTTT | 417 |
| OATP2, solute carrier family 21 member 6 | 349 | TTCCCTCTTTCTCTGCTTTCA | 418 |
| OATP2, solute carrier family 21 member 6 | 350 | TTCCCTCTTTCTCTGCTTTCA | 419 |
| OATP2, solute carrier family 21 member 6 | 351 | CATGCACATTCTGCACATGTA | 420 |
| OATP2, solute carrier family 21 member 6 | 352 | CATGCACATTCTGCACATGTA | 421 |
| OATP2, solute carrier family 21 member 6 | 353 | CATGCACATTCTGCACATGTA | 422 |
| OATP2, solute carrier family 21 member 6 | 354 | TTTGAACAAGTGAGACTTCACTAAA | 423 |
| OATP2, solute carrier family 21 member 6 | 355 | TGTTTAAAATGAAACACTCTCTTATCTACA | 424 |
| OATP2, solute carrier family 21 member 6 | 356 | TGTTTAAAATGAAACACTCTCTTATCTACA | 425 |
| OATP2, solute carrier family 21 member 6 | 357 | TGTTTAAAATGAAACACTCTCTTATCTACA | 426 |
| OATP2, solute carrier family 21 member 6 | 358 | CATATTTCTTTTAAAAACATGGTGAA | 427 |
| OATP2, solute carrier family 21 member 6 | 359 | AAAGTGAGAGACATGGTTACTGTG | 428 |
| OATP2, solute carrier family 21 member 6 | 360 | AAAGTGAGAGACATGGTTACTGTG | 429 |
| OATP2, solute carrier family 21 member 6 | 361 | AAAGTGAGAGACATGGTTACTGTG | 430 |
| OATP2, solute carrier family 21 member 6 | 362 | CTGGGGCAGATAGTGAAACAC | 431 |
| cMOAT, ATP-binding cassette sub-family C member 2 | 363 | GCAGGCTTTTGTCTTGTTCAG | 432 |
| cMOAT, ATP-binding cassette sub-family C member 2 | 364 | TTCTATGACACGAGTCCTGGG | 433 |
| cMOAT, ATP-binding cassette sub-family C member 2 | 365 | TCCTCACCAAAACCAAAATCA | 434 |
| cMOAT, ATP-binding cassette sub-family C member 2 | 366 | TCCTCACCAAAACCAAAATCA | 435 |
| cMOAT, ATP-binding cassette sub-family C member 2 | 367 | TCCTCACCAAAACCAAAATCA | 436 |
| cMOAT, ATP-binding cassette sub-family C member 2 | 368 | GGGGGTTTTGAAAGTCTGATC | 437 |
| cMOAT, ATP-binding cassette sub-family C member 2 | 369 | GGGGGTTTTGAAAGTCTGATC | 438 |
| cMOAT, ATP-binding cassette sub-family C member 2 | 370 | GGGGGTTTTGAAAGTCTGATC | 439 |
| cMOAT, ATP-binding cassette sub-family C member 2 | 371 | GGGGGTTTTGAAAGTCTGATC | 440 |
| cMOAT, ATP-binding cassette sub-family C member 2 | 372 | GCAGGCTTTTGTCTTGTTCAG | 441 |
| cMOAT, ATP-binding cassette sub-family C member 2 | 373 | CAAGAAGACCCTTGGGAATTG | 442 |
| cMOAT, ATP-binding cassette sub-family C member 2 | 374 | CAAGAAGACCCTTGGGAATTG | 443 |
| cMOAT, ATP-binding cassette sub-family C member 2 | 375 | TGTATGTATCCATTCTTTCCAGAA | 444 |
| cMOAT, ATP-binding cassette sub-family C member 2 | 376 | AGGCGCCTTCAACTCTGATAT | 445 |
| cMOAT, ATP-binding cassette sub-family C member 2 | 377 | ACCTCCTGTTAGCGTAGGAGC | 446 |
| cMOAT, ATP-binding cassette sub-family C member 2 | 378 | ACCTCCTGTTAGCGTAGGAGC | 447 |
| cMOAT, ATP-binding cassette sub-family C member 2 | 379 | CATGCTGTATGTACATCTGGGA | 448 |
| cMOAT, ATP-binding cassette sub-family C member 2 | 380 | CATGCTGTATGTACATCTGGGA | 449 |
| cMOAT, ATP-binding cassette sub-family C member 2 | 381 | GCCAGCTCTGTTGACATCTTT | 450 |
| cMOAT, ATP-binding cassette sub-family C member 2 | 382 | TGCCAGACTCTCAAATTCCAG | 451 |
| cMOAT, ATP-binding cassette sub-family C member 2 | 383 | AGATACACCTGGTGCCCTTTC | 452 |
| cMOAT, ATP-binding cassette sub-family C member 2 | 384 | AGGGGAAATCTCCTGATACCA | 453 |
| cMOAT, ATP-binding cassette sub-family C member 2 | 385 | TGTCCATGGGTCCTAATTTCA | 454 |
| cMOAT, ATP-binding cassette sub-family C member 2 | 386 | TGTCCATGGGTCCTAATTTCA | 455 |
| cMOAT, ATP-binding cassette sub-family C member 2 | 387 | CCCACTCCTCTGTCAAGTTCA | 456 |
| cMOAT, ATP-binding cassette sub-family C member 2 | 388 | ATGCAGCCTATTGCAAATGAA | 457 |
| cMOAT, ATP-binding cassette sub-family C member 2 | 389 | ATGCAGCCTATTGCAAATGAA | 458 |
| cMOAT, ATP-binding cassette sub-family C member 2 | 390 | ATGCAGCCTATTGCAAATGAA | 459 |
| cMOAT, ATP-binding cassette sub-family C member 2 | 391 | CCTCCTTTCTTCCCATGTTCT | 460 |
| cMOAT, ATP-binding cassette sub-family C member 2 | 392 | GATATAAAGGTGGGGTGGGAG | 461 |
| cMOAT, ATP-binding cassette sub-family C member 2 | 393 | GATATAAAGGTGGGGTGGGAG | 462 |
| cMOAT, ATP-binding cassette sub-family C member 2 | 394 | TTTTGCTACCTGCTACCCATG | 463 |
| cMOAT, ATP-binding cassette sub-family C member 2 | 395 | TATGTCCTGGGCACAAGTCTT | 464 |

TABLE IX

| GENE_DESCRIPTION | HGNC_ID | SNP_ID | EXON | REV_COMP | Target_Name |
|---|---|---|---|---|---|
| OATP2, solute carrier family 21 member 6 | SLC21A6 | PS100s1 | Exon5 | 1 | SLC21A6_X5a |
| OATP2, solute carrier family 21 member 6 | SLC21A6 | PS100s2 | Exon5 | 1 | SLC21A6_X5a |
| OATP2, solute carrier family 21 member 6 | SLC21A6 | PS100s3 | Intron5 | 1 | SLC21A6_X5a |
| OATP2, solute carrier family 21 member 6 | SLC21A6 | PS100s4 | Intron5 | 1 | SLC21A6_X5a |
| OATP2, solute carrier family 21 member 6 | SLC21A6 | PS100s5 | Intron5 | 1 | SLC21A6_X5a |
| OATP2, solute carrier family 21 member 6 | SLC21A6 | PS100s6 | Intron5 | 1 | SLC21A6_X5a |
| OATP2, solute carrier family 21 member 6 | SLC21A6 | PS100s7 | Exon15 | 1 | SLC21A6_X15_f2a |
| OATP2, solute carrier family 21 member 6 | SLC21A6 | PS100s8 | Intron5 | 1 | SLC21A6_X5a |
| OATP2, solute carrier family 21 member 6 | SLC21A6 | PS100s9 | Exon5 | 1 | SLC21A6_X5a |
| OATP2, solute carrier family 21 member 6 | SLC21A6 | PS100s10 | Intron1 | 1 | SLC21A6_X1a |
| OATP2, solute carrier family 21 member 6 | SLC21A6 | PS100s11 | Intron1 | 1 | SLC21A6_X1a |
| OATP2, solute carrier family 21 member 6 | SLC21A6 | PS100s12 | Intron1 | 1 | SLC21A6_X14a |
| OATP2, solute carrier family 21 member 6 | SLC21A6 | PS100s13 | Intron2 | 1 | SLC21A6_X2a |
| OATP2, solute carrier family 21 member 6 | SLC21A6 | PS100s14 | Intron2 | 1 | SLC21A6_X2a |
| OATP2, solute carrier family 21 member 6 | SLC21A6 | PS100s15 | Intron2 | 1 | SLC21A6_X2a |
| OATP2, solute carrier family 21 member 6 | SLC21A6 | PS100s16 | Intron2 | 1 | SLC21A6_X2a |
| OATP2, solute carrier family 21 member 6 | SLC21A6 | PS100s17 | Intron2 | 1 | SLC21A6_X2a |
| OATP2, solute carrier family 21 member 6 | SLC21A6 | PS100s18 | Intron2 | 1 | SLC21A6_X2a |
| OATP2, solute carrier family 21 member 6 | SLC21A6 | PS100s19 | Intron3 | 1 | SLC21A6_X3a |
| OATP2, solute carrier family 21 member 6 | SLC21A6 | PS100s20 | Intron3 | 1 | SLC21A6_X3a |
| OATP2, solute carrier family 21 member 6 | SLC21A6 | PS100s21 | Intron3 | 1 | SLC21A6_X3a |
| OATP2, solute carrier family 21 member 6 | SLC21A6 | PS100s22 | Intron3 | 1 | SLC21A6_X3a |
| OATP2, solute carrier family 21 member 6 | SLC21A6 | PS100s23 | Exon11 | 1 | SLC21A6_X11a |
| OATP2, solute carrier family 21 member 6 | SLC21A6 | PS100s24 | Intron11 | 1 | SLC21A6_X11a |
| OATP2, solute carrier family 21 member 6 | SLC21A6 | PS100s25 | Exon10 | 1 | SLC21A6_X10 |
| OATP2, solute carrier family 21 member 6 | SLC21A6 | PS100s26 | Exon10 | 1 | SLC21A6_X10 |
| OATP2, solute carrier family 21 member 6 | SLC21A6 | PS100s27 | Intron9 | 1 | SLC21A6_X10 |
| OATP2, solute carrier family 21 member 6 | SLC21A6 | PS100s28 | Intron11 | 1 | SLC21A6_X12a |
| OATP2, solute carrier family 21 member 6 | SLC21A6 | PS100s29 | Exon6 | 1 | SLC21A6_X6a |
| OATP2, solute carrier family 21 member 6 | SLC21A6 | PS100s30 | Exon6 | 1 | SLC21A6_X6a |
| OATP2, solute carrier family 21 member 6 | SLC21A6 | PS100s31 | Exon6 | 1 | SLC21A6_X6a |
| OATP2, solute carrier family 21 member 6 | SLC21A6 | PS100s32 | Intron7 | 1 | SLC21A6_X7a |
| OATP2, solute carrier family 21 member 6 | SLC21A6 | PS100s33 | Exon15 | 1 | SLC21A6_X15_f1a |
| OATP2, solute carrier family 21 member 6 | SLC21A6 | PS100s34 | Exon15 | 1 | SLC21A6_X15_f1a |
| OATP2, solute carrier family 21 member 6 | SLC21A6 | PS100s35 | Exon15 | 1 | SLC21A6_X15_f1a |
| OATP2, solute carrier family 21 member 6 | SLC21A6 | PS100s36 | Exon15 | 1 | SLC21A6_X15_f2a |
| cMOAT, ATP-binding cassette sub-family C member 2 | ABCC2 | PS101s1 | Exon25 | 1 | ABCC2_X25a |
| cMOAT, ATP-binding cassette sub-family C member 2 | ABCC2 | PS101s2 | Exon28 | 1 | ABCC2_X28a |
| cMOAT, ATP-binding cassette sub-family C member 2 | ABCC2 | PS101s3 | Intron29 | 1 | ABCC2_X29a |
| cMOAT, ATP-binding cassette sub-family C member 2 | ABCC2 | PS101s4 | Exon29 | 1 | ABCC2_X29a |
| cMOAT, ATP-binding cassette sub-family C member 2 | ABCC2 | PS101s5 | Exon29 | 1 | ABCC2_X29a |
| cMOAT, ATP-binding cassette sub-family C member 2 | ABCC2 | PS101s6 | Exon31 | 1 | ABCC2_X31a |
| cMOAT, ATP-binding cassette sub-family C member 2 | ABCC2 | PS101s7 | Exon31 | 1 | ABCC2_X31a |
| cMOAT, ATP-binding cassette sub-family C member 2 | ABCC2 | PS101s8 | Intron31 | 1 | ABCC2_X31a |
| cMOAT, ATP-binding cassette sub-family C member 2 | ABCC2 | PS101s9 | Intron31 | 1 | ABCC2_X31a |
| cMOAT, ATP-binding cassette sub-family C member 2 | ABCC2 | PS101s10 | Exon25 | 1 | ABCC2_X25a |
| cMOAT, ATP-binding cassette sub-family C member 2 | ABCC2 | PS101s11 | Exon21 | 1 | ABCC2_X21a |
| cMOAT, ATP-binding cassette sub-family C member 2 | ABCC2 | PS101s12 | Intron21 | 1 | ABCC2_X21a |
| cMOAT, ATP-binding cassette sub-family C member 2 | ABCC2 | PS101s13 | Exon3 | 1 | ABCC2_X3a |
| cMOAT, ATP-binding cassette sub-family C member 2 | ABCC2 | PS101s14 | Intron12 | 1 | ABCC2_X13a |
| cMOAT, ATP-binding cassette sub-family C member 2 | ABCC2 | PS101s15 | Intron15 | 1 | ABCC2_X15a |
| cMOAT, ATP-binding cassette sub-family C member 2 | ABCC2 | PS101s16 | Intron15 | 1 | ABCC2_X15a |
| cMOAT, ATP-binding cassette sub-family C member 2 | ABCC2 | PS101s17 | Intron15 | 1 | ABCC2_X20a |
| cMOAT, ATP-binding cassette sub-family C member 2 | ABCC2 | PS101s18 | Intron15 | 1 | ABCC2_X20a |
| cMOAT, ATP-binding cassette sub-family C member 2 | ABCC2 | PS101s19 | Exon1 | 1 | ABCC2_X1a |
| cMOAT, ATP-binding cassette sub-family C member 2 | ABCC2 | PS101s20 | Intron20 | 1 | ABCC2_X27a |
| cMOAT, ATP-binding cassette sub-family C member 2 | ABCC2 | PS101s21 | Intron12 | 1 | ABCC2_X12a |
| cMOAT, ATP-binding cassette sub-family C member 2 | ABCC2 | PS101s22 | Exon16 | 1 | ABCC2_X16a |
| cMOAT, ATP-binding cassette sub-family C member 2 | ABCC2 | PS101s23 | Exon10 | 1 | ABCC2_X10a |
| cMOAT, ATP-binding cassette sub-family C member 2 | ABCC2 | PS101s24 | Exon10 | 1 | ABCC2_X10a |
| cMOAT, ATP-binding cassette sub-family C member 2 | ABCC2 | PS101s25 | Intron6 | 1 | ABCC2_X7a |
| cMOAT, ATP-binding cassette sub-family C member 2 | ABCC2 | PS101s26 | Intron24 | 1 | ABCC2_X24a |
| cMOAT, ATP-binding cassette sub-family C member 2 | ABCC2 | PS101s27 | Intron24 | 1 | ABCC2_X24a |
| cMOAT, ATP-binding cassette sub-family C member 2 | ABCC2 | PS101s28 | Intron24 | 1 | ABCC2_X24a |
| cMOAT, ATP-binding cassette sub-family C member 2 | ABCC2 | PS101s29 | Intron3 | 1 | ABCC2_X4a |
| cMOAT, ATP-binding cassette sub-family C member 2 | ABCC2 | PS101s30 | Intron19 | 1 | ABCC2_X19a |
| cMOAT, ATP-binding cassette sub-family C member 2 | ABCC2 | PS101s31 | Intron19 | 1 | ABCC2_X19a |
| cMOAT, ATP-binding cassette sub-family C member 2 | ABCC2 | PS101s32 | Exon22 | 1 | ABCC2_X22a |
| cMOAT, ATP-binding cassette sub-family C member 2 | ABCC2 | PS101s33 | Intron23 | 1 | ABCC2_X23a |

| | | | Forward |
|---|---|---|---|
| | | | Sequencing |
| | | Forward_Seq_{—} | Primer |
| GENE_DESCRIPTION | Forward Sequencing Primer | Name | (SEQ ID NO:) |
| OATP2, solute carrier family 21 member 6 | CTGTGTTGTTAATGGGCGAAC | PS100p51 | 465 |
| OATP2, solute carrier family 21 member 6 | CTGTGTTGTTAATGGGCGAAC | PS100p51 | 466 |
| OATP2, solute carrier family 21 member 6 | CTGTGTTGTTAATGGGCGAAC | PS100p51 | 467 |
| OATP2, solute carrier family 21 member 6 | CTGTGTTGTTAATGGGCGAAC | PS100p51 | 468 |
| OATP2, solute carrier family 21 member 6 | CTGTGTTGTTAATGGGCGAAC | PS100p51 | 469 |
| OATP2, solute carrier family 21 member 6 | CTGTGTTGTTAATGGGCGAAC | PS100p51 | 470 |
| OATP2, solute carrier family 21 member 6 | TTCAAACTCTGTGTTTGTTTGTTT | PS100p7 | 471 |
| OATP2, solute carrier family 21 member 6 | CTGTGTTGTTAATGGGCGAAC | PS100p51 | 472 |
| OATP2, solute carrier family 21 member 6 | CTGTGTTGTTAATGGGCGAAC | PS100p51 | 473 |
| OATP2, solute carrier family 21 member 6 | AAGGGCTCAGAATGTAAGCGT | PS100p67 | 474 |
| OATP2, solute carrier family 21 member 6 | AAGGGCTCAGAATGTAAGCGT | PS100p67 | 475 |
| OATP2, solute carrier family 21 member 6 | CCATACTGCATGCAAAGTCAG | PS100p15 | 476 |
| OATP2, solute carrier family 21 member 6 | CATCAAAAATTTCGCCAAAGA | PS100p63 | 477 |
| OATP2, solute carrier family 21 member 6 | CATCAAAAATTTCGCCAAAGA | PS100p63 | 478 |
| OATP2, solute carrier family 21 member 6 | CATCAAAAATTTCGCCAAAGA | PS100p63 | 479 |
| OATP2, solute carrier family 21 member 6 | CATCAAAAATTTCGCCAAAGA | PS100p63 | 480 |
| OATP2, solute carrier family 21 member 6 | CATCAAAAATTTCGCCAAAGA | PS100p63 | 481 |
| OATP2, solute carrier family 21 member 6 | CATCAAAAATTTCGCCAAAGA | PS100p63 | 482 |
| OATP2, solute carrier family 21 member 6 | GCAGTACCCCTGACCTCTACC | PS100p59 | 483 |
| OATP2, solute carrier family 21 member 6 | GCAGTACCCCTGACCTCTACC | PS100p59 | 484 |
| OATP2, solute carrier family 21 member 6 | GCAGTACCCCTGACCTCTACC | PS100p59 | 485 |
| OATP2, solute carrier family 21 member 6 | GCAGTACCCCTGACCTCTACC | PS100p59 | 486 |
| OATP2, solute carrier family 21 member 6 | CATCACACCCATCACAATAACA | PS100p27 | 487 |
| OATP2, solute carrier family 21 member 6 | CATCACACCCATCACAATAACA | PS100p27 | 488 |
| OATP2, solute carrier family 21 member 6 | TTTGATGGTTAACATATTATGCAATT | PS100p31 | 489 |
| OATP2, solute carrier family 21 member 6 | TTTGATGGTTAACATATTATGCAATT | PS100p31 | 490 |
| OATP2, solute carrier family 21 member 6 | TTTGATGGTTAACATATTATGCAATT | PS100p31 | 491 |
| OATP2, solute carrier family 21 member 6 | TGAAATATGAATTTGAATTCCCAG | PS100p23 | 492 |
| OATP2, solute carrier family 21 member 6 | GTAAAGCCAATGATTGGACCA | PS100p47 | 493 |
| OATP2, solute carrier family 21 member 6 | GTAAAGCCAATGATTGGACCA | PS100p47 | 494 |
| OATP2, solute carrier family 21 member 6 | GTAAAGCCAATGATTGGACCA | PS100p47 | 495 |
| OATP2, solute carrier family 21 member 6 | TGTTTTTCGCATGTGTGCTTA | PS100p43 | 496 |
| OATP2, solute carrier family 21 member 6 | GCCCACTGGAAACTTAACACA | PS100p3 | 497 |
| OATP2, solute carrier family 21 member 6 | GCCCACTGGAAACTTAACACA | PS100p3 | 498 |
| OATP2, solute carrier family 21 member 6 | GCCCACTGGAAACTTAACACA | PS100p3 | 499 |
| OATP2, solute carrier family 21 member 6 | TTCAAACTCTGTGTTTGTTTGTTT | PS100p7 | 500 |
| cMOAT, ATP-binding cassette sub-family C member 2 | ATTTCACACCACTAGCCATGC | PS101p35 | 501 |
| cMOAT, ATP-binding cassette sub-family C member 2 | ACAGCTGCGGTAAGTCTGTGT | PS101p23 | 502 |
| cMOAT, ATP-binding cassette sub-family C member 2 | TGGAATTGACAGTGCTGACTG | PS101p19 | 503 |
| cMOAT, ATP-binding cassette sub-family C member 2 | TGGAATTGACAGTGCTGACTG | PS101p19 | 504 |
| cMOAT, ATP-binding cassette sub-family C member 2 | TGGAATTGACAGTGCTGACTG | PS101p19 | 505 |
| cMOAT, ATP-binding cassette sub-family C member 2 | TAAACCTTCTGCCATCAGGTG | PS101p11 | 506 |
| cMOAT, ATP-binding cassette sub-family C member 2 | TAAACCTTCTGCCATCAGGTG | PS101p11 | 507 |
| cMOAT, ATP-binding cassette sub-family C member 2 | TAAACCTTCTGCCATCAGGTG | PS101p11 | 508 |
| cMOAT, ATP-binding cassette sub-family C member 2 | TAAACCTTCTGCCATCAGGTG | PS101p11 | 509 |
| cMOAT, ATP-binding cassette sub-family C member 2 | ATTTCACACCACTAGCCATGC | PS101p35 | 510 |
| cMOAT, ATP-binding cassette sub-family C member 2 | GGATGAGGACATTCACAGGAA | PS101p51 | 511 |
| cMOAT, ATP-binding cassette sub-family C member 2 | GGATGAGGACATTCACAGGAA | PS101p51 | 512 |
| cMOAT, ATP-binding cassette sub-family C member 2 | AAATTTGTTTCACCCCATTCC | PS101p123 | 513 |
| cMOAT, ATP-binding cassette sub-family C member 2 | GTCAAACCATTGGTCTCCAGA | PS101p83 | 514 |
| cMOAT, ATP-binding cassette sub-family C member 2 | ATGGCAAATGCAGTTATCAGG | PS101p75 | 515 |
| cMOAT, ATP-binding cassette sub-family C member 2 | ATGGCAAATGCAGTTATCAGG | PS101p75 | 516 |
| cMOAT, ATP-binding cassette sub-family C member 2 | AAGGAAGAGCTTGGAGTGTCC | PS101p55 | 517 |
| cMOAT, ATP-binding cassette sub-family C member 2 | AAGGAAGAGCTTGGAGTGTCC | PS101p55 | 518 |
| cMOAT, ATP-binding cassette sub-family C member 2 | TTCTGGTTCTTGTTGGTGACC | PS101p131 | 519 |
| cMOAT, ATP-binding cassette sub-family C member 2 | GATGCACTCTCGAAGGAGTTG | PS101p27 | 520 |
| cMOAT, ATP-binding cassette sub-family C member 2 | GGCCTAGAGATGCCAGCTAGT | PS101p87 | 521 |
| cMOAT, ATP-binding cassette sub-family C member 2 | ACTAGCCCTCAGTGCCTTCTC | PS101p71 | 522 |
| cMOAT, ATP-binding cassette sub-family C member 2 | GCCCAAACTCCCATTAAGAAT | PS101p95 | 523 |
| cMOAT, ATP-binding cassette sub-family C member 2 | GCCCAAACTCCCATTAAGAAT | PS101p95 | 524 |
| cMOAT, ATP-binding cassette sub-family C member 2 | TGATGTACCCTTGCCTGAAAC | PS101p107 | 525 |
| cMOAT, ATP-binding cassette sub-family C member 2 | ACTAAATGGCAAAGCTGCTGA | PS101p39 | 526 |
| cMOAT, ATP-binding cassette sub-family C member 2 | ACTAAATGGCAAAGCTGCTGA | PS101p39 | 527 |
| cMOAT, ATP-binding cassette sub-family C member 2 | ACTAAATGGCAAAGCTGCTGA | PS101p39 | 528 |
| cMOAT, ATP-binding cassette sub-family C member 2 | GCCTTTTGTCCAAAGGAAGTC | PS101p119 | 529 |
| cMOAT, ATP-binding cassette sub-family C member 2 | CACAGAACCCAGAAAGCAGAG | PS101p59 | 530 |
| cMOAT, ATP-binding cassette sub-family C member 2 | CACAGAACCCAGAAAGCAGAG | PS101p59 | 531 |
| cMOAT, ATP-binding cassette sub-family C member 2 | ACTTGTGCCCAGGACATAATG | PS101p47 | 532 |
| cMOAT, ATP-binding cassette sub-family C member 2 | TGAACAGTGTTGTCTAGGGGG | PS101p43 | 533 |

| | | | Reverse |
|---|---|---|---|
| | | | Sequencing |
| | | Reverse_{—} | Primer (SEQ |
| | | Seq_{—} | ID |
| GENE_DESCRIPTION | Reverse Sequencing Primer | Name | NO:) |
| OATP2, solute carrier family 21 member 6 | ATGGTGCAAATAAAGGGGAAT | PS100p52 | 534 |
| OATP2, solute carrier family 21 member 6 | ATGGTGCAAATAAAGGGGAAT | PS100p52 | 535 |
| OATP2, solute carrier family 21 member 6 | ATGGTGCAAATAAAGGGGAAT | PS100p52 | 536 |
| OATP2, solute carrier family 21 member 6 | ATGGTGCAAATAAAGGGGAAT | PS100p52 | 537 |
| OATP2, solute carrier family 21 member 6 | ATGGTGCAAATAAAGGGGAAT | PS100p52 | 538 |
| OATP2, solute carrier family 21 member 6 | ATGGTGCAAATAAAGGGGAAT | PS100p52 | 539 |
| OATP2, solute carrier family 21 member 6 | GTTTCCAAACAGCATTGCATT | PS100p8 | 540 |
| OATP2, solute carrier family 21 member 6 | ATGGTGCAAATAAAGGGGAAT | PS100p52 | 541 |
| OATP2, solute carrier family 21 member 6 | ATGGTGCAAATAAAGGGGAAT | PS100p52 | 542 |
| OATP2, solute carrier family 21 member 6 | TGGCAACTGGAGTGAACTCTT | PS100p68 | 543 |
| OATP2, solute carrier family 21 member 6 | TGGCAACTGGAGTGAACTCTT | PS100p68 | 544 |
| OATP2, solute carrier family 21 member 6 | CGAATCCTCCAAATTTTTGAAC | PS100p16 | 545 |
| OATP2, solute carrier family 21 member 6 | AGGTGATTGTTTCAAACTGAGC | PS100p64 | 546 |
| OATP2, solute carrier family 21 member 6 | AGGTGATTGTTTCAAACTGAGC | PS100p64 | 547 |
| OATP2, solute carrier family 21 member 6 | AGGTGATTGTTTCAAACTGAGC | PS100p64 | 548 |
| OATP2, solute carrier family 21 member 6 | AGGTGATTGTTTCAAACTGAGC | PS100p64 | 549 |
| OATP2, solute carrier family 21 member 6 | AGGTGATTGTTTCAAACTGAGC | PS100p64 | 550 |
| OATP2, solute carrier family 21 member 6 | AGGTGATTGTTTCAAACTGAGC | PS100p64 | 551 |
| OATP2, solute carrier family 21 member 6 | GATGTTCTTGGCAGCTCTGTC | PS100p60 | 552 |
| OATP2, solute carrier family 21 member 6 | GATGTTCTTGGCAGCTCTGTC | PS100p60 | 553 |
| OATP2, solute carrier family 21 member 6 | GATGTTCTTGGCAGCTCTGTC | PS100p60 | 554 |
| OATP2, solute carrier family 21 member 6 | GATGTTCTTGGCAGCTCTGTC | PS100p60 | 555 |
| OATP2, solute carrier family 21 member 6 | CTTCTTCCTTCTCCTCCCCTT | PS100p28 | 556 |
| OATP2, solute carrier family 21 member 6 | CTTCTTCCTTCTCCTCCCCTT | PS100p28 | 557 |
| OATP2, solute carrier family 21 member 6 | CATGCACATTCTGCACATGTA | PS100p32 | 558 |
| OATP2, solute carrier family 21 member 6 | CATGCACATTCTGCACATGTA | PS100p32 | 559 |
| OATP2, solute carrier family 21 member 6 | CATGCACATTCTGCACATGTA | PS100p32 | 560 |
| OATP2, solute carrier family 21 member 6 | TGCAATGTATTTGCAGCACTG | PS100p24 | 561 |
| OATP2, solute carrier family 21 member 6 | GGTTGTTTAAAGGAATCTGGG | PS100p48 | 562 |
| OATP2, solute carrier family 21 member 6 | GGTTGTTTAAAGGAATCTGGG | PS100p48 | 563 |
| OATP2, solute carrier family 21 member 6 | GGTTGTTTAAAGGAATCTGGG | PS100p48 | 564 |
| OATP2, solute carrier family 21 member 6 | TAAGAACCATGCATTCTTGGC | PS100p44 | 565 |
| OATP2, solute carrier family 21 member 6 | AAACAAATGAGTATCATACAGGTAGAGG | PS100p4 | 566 |
| OATP2, solute carrier family 21 member 6 | AAACAAATGAGTATCATACAGGTAGAGG | PS100p4 | 567 |
| OATP2, solute carrier family 21 member 6 | AAACAAATGAGTATCATACAGGTAGAGG | PS100p4 | 568 |
| OATP2, solute carrier family 21 member 6 | GTTTCCAAACAGCATTGCATT | PS100p8 | 569 |
| cMOAT, ATP-binding cassette sub-family C member 2 | AGAAAGGAGGAAGATGGTGGA | PS101p3 | 570 |
| cMOAT, ATP-binding cassette sub-family C member 2 | CTCATCTTGTCTCCTTGCCAG | PS101p24 | 571 |
| cMOAT, ATP-binding cassette sub-family C member 2 | CACTGCCTCTTACCTCCTGTG | PS101p20 | 572 |
| cMOAT, ATP-binding cassette sub-family C member 2 | CACTGCCTCTTACCTCCTGTG | PS101p20 | 573 |
| cMOAT, ATP-binding cassette sub-family C member 2 | CACTGCCTCTTACCTCCTGTG | PS101p20 | 574 |
| cMOAT, ATP-binding cassette sub-family C member 2 | TGCGTCTTTCCTTGGTCTTTA | PS101p12 | 575 |
| cMOAT, ATP-binding cassette sub-family C member 2 | TGCGTCTTTCCTTGGTCTTTA | PS101p12 | 576 |
| cMOAT, ATP-binding cassette sub-family C member 2 | TGCGTCTTTCCTTGGTCTTTA | PS101p12 | 577 |
| cMOAT, ATP-binding cassette sub-family C member 2 | TGCGTCTTTCCTTGGTCTTTA | PS101p12 | 578 |
| cMOAT, ATP-binding cassette sub-family C member 2 | AGAAAGGAGGAAGATGGTGGA | PS101p36 | 579 |
| cMOAT, ATP-binding cassette sub-family C member 2 | CAGTTCTAGGTCCAATGGCAG | PS101p52 | 580 |
| cMOAT, ATP-binding cassette sub-family C member 2 | CAGTTCTAGGTCCAATGGCAG | PS101p52 | 581 |
| cMOAT, ATP-binding cassette sub-family C member 2 | TCACTGCATACCGTTTTTCCT | PS101p124 | 582 |
| cMOAT, ATP-binding cassette sub-family C member 2 | TGCTTGGTCCCTTTTAGGAAT | PS101p84 | 583 |
| cMOAT, ATP-binding cassette sub-family C member 2 | ATGGAGAAAGCGGAGAGAGAC | PS101p76 | 584 |
| cMOAT, ATP-binding cassette sub-family C member 2 | ATGGAGAAAGCGGAGAGAGAC | PS101p76 | 585 |
| cMOAT, ATP-binding cassette sub-family C member 2 | CTTGCTGAAACCAGCAAGATC | PS101p56 | 586 |
| cMOAT, ATP-binding cassette sub-family C member 2 | CTTGCTGAAACCAGCAAGATC | PS101p56 | 587 |
| cMOAT, ATP-binding cassette sub-family C member 2 | GGCCTCTTGTACTTTGGGAAC | PS101p132 | 588 |
| cMOAT, ATP-binding cassette sub-family C member 2 | CGCGTGAGCACCTAATTAGTC | PS101p28 | 589 |
| cMOAT, ATP-binding cassette sub-family C member 2 | GGGCAATCATGTGAGCTGTAT | PS101p88 | 590 |
| cMOAT, ATP-binding cassette sub-family C member 2 | ACCCCTGCTATCTCCTTCAAA | PS101p72 | 591 |
| cMOAT, ATP-binding cassette sub-family C member 2 | AGGCATTGACCCTATCCAACT | PS101p96 | 592 |
| cMOAT, ATP-binding cassette sub-family C member 2 | AGGCATTGACCCTATCCAACT | PS101p96 | 593 |
| cMOAT, ATP-binding cassette sub-family C member 2 | CTGTCCCTCTATCCCAGAACC | PS101p108 | 594 |
| cMOAT, ATP-binding cassette sub-family C member 2 | ACTGGGAACACACAGAATCCA | PS101p40 | 595 |
| cMOAT, ATP-binding cassette sub-family C member 2 | ACTGGGAACACACAGAATCCA | PS101p40 | 596 |
| cMOAT, ATP-binding cassette sub-family C member 2 | ACTGGGAACACACAGAATCCA | PS101p40 | 597 |
| cMOAT, ATP-binding cassette sub-family C member 2 | GACATCCTTCTCCCCTCAGTC | PS101p120 | 598 |
| cMOAT, ATP-binding cassette sub-family C member 2 | TGGCAAGTAAGACAGGGAAGA | PS101p60 | 599 |
| cMOAT, ATP-binding cassette sub-family C member 2 | TGGCAAGTAAGACAGGGAAGA | PS101p60 | 600 |
| cMOAT, ATP-binding cassette sub-family C member 2 | CTCCTTGTGGTTGGCATTCTA | PS101p48 | 601 |
| cMOAT, ATP-binding cassette sub-family C member 2 | GCTGACAAAACTGCTTCCATC | PS101p44 | 602 |

TABLE X

| | | ORCHID_{—} | |
|---|---|---|---|
| | | LEFT (SEQ | |
| SNP_ID | ORCHID_LEFT | ID NO:) | ORCHID_RIGHT |
| PS100s1 | N/A | N/A | N/A |
| PS100s2 | TGCTAATGAATATCACAACAATTTTTAGAG | 620 | TCGACCTTATCCACTTGTTTAATT |
| PS100s3 | N/A | N/A | N/A |
| PS100s4 | N/A | N/A | N/A |
| PS100s5 | N/A | N/A | N/A |
| PS100s6 | N/A | N/A | N/A |
| PS100s7 | N/A | N/A | N/A |
| PS100s8 | N/A | N/A | N/A |
| PS100s9 | GGTGATGCTCTATTGAGTGATAAAATTT | 621 | GTGATGTTCTTACAGTTACAGGTATTCTAAAG |
| PS100s10 | N/A | N/A | N/A |
| PS100s11 | N/A | N/A | N/A |
| PS100s12 | N/A | N/A | N/A |
| PS100s13 | N/A | N/A | N/A |
| PS100s14 | N/A | N/A | N/A |
| PS100s15 | N/A | N/A | N/A |
| PS100s16 | N/A | N/A | N/A |
| PS100s17 | N/A | N/A | N/A |
| PS100s18 | N/A | N/A | N/A |
| PS100s19 | N/A | N/A | N/A |
| PS100s20 | N/A | N/A | N/A |
| PS100s21 | N/A | N/A | N/A |
| PS100s22 | N/A | N/A | N/A |
| PS100s23 | TTTTGAATAAGGAGAGGAAAGTAAAA | 622 | GAATAACTTACATCTCACCCTGTCTAG |
| PS100s24 | N/A | N/A | N/A |
| PS100s25 | N/A | N/A | N/A |
| PS100s26 | GACATCACAGCAGTAAAACATGAGA | 623 | AGGAGTCATAACCATACCTATTTTTGC |
| PS100s27 | N/A | N/A | N/A |
| PS100s28 | N/A | N/A | N/A |
| PS100s29 | CCCAATGGTACTATGGGAGTCTC | 624 | CATAGGTTGTTTAAAGGAATCTGGG |
| PS100s30 | CATTACCTAAATACAAAGAAGAATGTCCTT | 625 | TGGGTAATATGCTTCGTGGAATAG |
| PS100s31 | N/A | N/A | N/A |
| PS100s32 | N/A | N/A | N/A |
| PS100s33 | N/A | N/A | N/A |
| PS100s34 | N/A | N/A | N/A |
| PS100s35 | N/A | N/A | N/A |
| PS100s36 | N/A | N/A | N/A |
| PS101s1 | CTCACCTGTTGGAGGTGATCC | 626 | GTATCAGGTTTGCCAGTTATCCG |
| PS101s2 | N/A | N/A | N/A |
| PS101s3 | N/A | N/A | N/A |
| PS101s4 | N/A | N/A | N/A |
| PS101s5 | N/A | N/A | N/A |
| PS101s6 | N/A | N/A | N/A |
| PS101s7 | N/A | N/A | N/A |
| PS101s8 | N/A | N/A | N/A |
| PS101s9 | N/A | N/A | N/A |
| PS101s10 | N/A | N/A | N/A |
| PS101s11 | N/A | N/A | N/A |
| PS101s12 | N/A | N/A | N/A |
| PS101s13 | N/A | N/A | N/A |
| PS101s14 | N/A | N/A | N/A |
| PS101s15 | N/A | N/A | N/A |
| PS101s16 | N/A | N/A | N/A |
| PS101s17 | N/A | N/A | N/A |
| PS101s18 | N/A | N/A | N/A |
| PS101s19 | N/A | N/A | N/A |
| PS101s20 | N/A | N/A | N/A |
| PS101s21 | N/A | N/A | N/A |
| PS101s22 | ATTTTCCATTTCTCCCAGCAT | 627 | CTTTCAGTGTGAACCTGGACATTAT |
| PS101s23 | TGCATGAAGTTGGTCACATCC | 628 | CCTAATTTCAATCCTTATCTTTAGGCA |
| PS101s24 | N/A | N/A | N/A |
| PS101s25 | N/A | N/A | N/A |
| PS101s26 | N/A | N/A | N/A |
| PS101s27 | N/A | N/A | N/A |
| PS101s28 | N/A | N/A | N/A |
| PS101s29 | N/A | N/A | N/A |
| PS101s30 | N/A | N/A | N/A |
| PS101s31 | N/A | N/A | N/A |
| PS101s32 | N/A | N/A | N/A |
| PS101s33 | N/A | N/A | N/A |

| SNP_ID | ORCHID_RIGHT (SEQ ID NO:) | ORCHID_SNPIT | ORCHID_SNPIT (SEQ ID NO:) |
|---|---|---|---|
| PS100s1 | N/A | N/A | N/A |
| PS100s2 | 629 | TTCTTACCTTTTCCCACTATCTCAG | 638 |
| PS100s3 | N/A | N/A | N/A |
| PS100s4 | N/A | N/A | N/A |
| PS100s5 | N/A | N/A | N/A |
| PS100s6 | N/A | N/A | N/A |
| PS100s7 | N/A | N/A | N/A |
| PS100s8 | N/A | N/A | N/A |
| PS100s9 | 630 | GTCGATGTTGAATTTTCTGATGAAT | 639 |
| PS100s10 | N/A | N/A | N/A |
| PS100s11 | N/A | N/A | N/A |
| PS100s12 | N/A | N/A | N/A |
| PS100s13 | N/A | N/A | N/A |
| PS100s14 | N/A | N/A | N/A |
| PS100s15 | N/A | N/A | N/A |
| PS100s16 | N/A | N/A | N/A |
| PS100s17 | N/A | N/A | N/A |
| PS100s18 | N/A | N/A | N/A |
| PS100s19 | N/A | N/A | N/A |
| PS100s20 | N/A | N/A | N/A |
| PS100s21 | N/A | N/A | N/A |
| PS100s22 | N/A | N/A | N/A |
| PS100s23 | 631 | TTTATTGCCACTTGAAGATTTGCAA | 640 |
| PS100s24 | N/A | N/A | N/A |
| PS100s25 | N/A | N/A | N/A |
| PS100s26 | 632 | TGGCAATTCCAACGGTGTTCAGTTT | 641 |
| PS100s27 | N/A | N/A | N/A |
| PS100s28 | N/A | N/A | N/A |
| PS100s29 | 633 | N/A | N/A |
| PS100s30 | 634 | AAATCATCAATGTAAGAAAGCCCCA | 642 |
| PS100s31 | N/A | N/A | N/A |
| PS100s32 | N/A | N/A | N/A |
| PS100s33 | N/A | N/A | N/A |
| PS100s34 | N/A | N/A | N/A |
| PS100s35 | N/A | N/A | N/A |
| PS100s36 | N/A | N/A | N/A |
| PS101s1 | 635 | ACATTTCTGGTTGGTGTCAATCCTC | 643 |
| PS101s2 | N/A | N/A | N/A |
| PS101s3 | N/A | N/A | N/A |
| PS101s4 | N/A | N/A | N/A |
| PS101s5 | N/A | N/A | N/A |
| PS101s6 | N/A | N/A | N/A |
| PS101s7 | N/A | N/A | N/A |
| PS101s8 | N/A | N/A | N/A |
| PS101s9 | N/A | N/A | N/A |
| PS101s10 | N/A | N/A | N/A |
| PS101s11 | N/A | N/A | N/A |
| PS101s12 | N/A | N/A | N/A |
| PS101s13 | N/A | N/A | N/A |
| PS101s14 | N/A | N/A | N/A |
| PS101s15 | N/A | N/A | N/A |
| PS101s16 | N/A | N/A | N/A |
| PS101s17 | N/A | N/A | N/A |
| PS101s18 | N/A | N/A | N/A |
| PS101s19 | N/A | N/A | N/A |
| PS101s20 | N/A | N/A | N/A |
| PS101s21 | N/A | N/A | N/A |
| PS101s22 | 636 | TTATGGCAGGNCAACTTGTGGCTGTGA | 644 |
| PS101s23 | 637 | GACATCAGGTTCACTGTTTCTCCAA | 645 |
| PS101s24 | N/A | N/A | N/A |
| PS101s25 | N/A | N/A | N/A |
| PS101s26 | N/A | N/A | N/A |
| PS101s27 | N/A | N/A | N/A |
| PS101s28 | N/A | N/A | N/A |
| PS101s29 | N/A | N/A | N/A |
| PS101s30 | N/A | N/A | N/A |
| PS101s31 | N/A | N/A | N/A |
| PS101s32 | N/A | N/A | N/A |
| PS101s33 | N/A | N/A | N/A |

TABLE XI

| | | GBS_LEFT | | GBS_RIGHT |
|---|---|---|---|---|
| SNP_ID | GBS_LEFT | (SEQ ID NO:) | GBS_RIGHT | (SEQ ID NO:) |
| PS100s1 | TGTAAAACGACGGCCAGTCTGTGTTGTTAATGGGCGAAAC | 646 | CAGGAAACAGCTATGACCATGGTGCAAATAAAGGGGAAT | 715 |
| PS100s2 | TGTAAAACGACGGCCAGTCTGTGTTGTTAATGGGCGAAAC | 647 | CAGGPAACAGCTATGACCATGGTGCAAATMAGGGGAAT | 716 |
| PS100s3 | TGTAAAACGACGGCCAGTGGCCCTGTTCAATCCAAATAT | 648 | CAGGWCAGCTATGACCGCATCACCTGAGATAGTGGGA | 717 |
| PS100s4 | TGTAAAACGACGGCCAGTGGCCCTGTTCAATCCAAATAT | 649 | CAGGAAACAGCTATGACCGCATCACCTGAGATAGTGGGA | 718 |
| PS100s5 | TGTAAAACGACGGCCAGTGAATTTGTTACAGGGCTGCCT | 650 | CAGGAAACAGCTATGACGGCATCACCTGAGATAGTGGGA | 719 |
| PS100s6 | TGTPAAACGACGGCCAGTGAATTTGTTACAGGGCTGCCT | 651 | CAGGAAACAGCTATGACCGCATCACCTGAGATAGTGGGA | 720 |
| PS100s7 | TGTAAAACGACGGCCAGTGCTCCTCCTTTTTAACCTCTACC | 652 | CAGGAAACAGCTATGACCGTTTCCAAAGAGCATTGCATT | 721 |
| PS100s8 | TGTAAAACGACGGCCAGTCTGTGTTGTTAATGGGCGAAC | 653 | CAGGAAACAGCTATGACCATGGTGCAAATAAAGGGGAAT | 722 |
| PS100s9 | TGTAAAACGACGGCCAGTCTGTGTTGTTAATGGGCGAAC | 654 | CAGGAAACAGCTATGACCATGGTGGAAATAAAGGGGAAT | 723 |
| PS100s10 | TGTAAAACGACGGCCAGTGGCTCTTCTACTCCCAGAAGG | 655 | CAGGAMCAGCTATGACCTGGCAACTGGAGTGMCTCTT | 724 |
| PS1G0s11 | TGTAAAACGACGGCCAGTACCTGAGGCTCTTCTACTCCC | 656 | CAGGAAACAGCTATGACCTGGCAACTGGAGTGAACTCTT | 725 |
| PS100s12 | TGTAAAACGACGGCCAGTTCCTTGAATCACAGTTTGTTCG | 657 | CAGGAAACAGCTATGACCTGGGGCTCGATTGATACAAC | 726 |
| PS1Q0s13 | TGTAAAACGACGGCCAGTCGTGATCAATCCAAAACCAAA | 658 | CAGGAAACAGCTATGACCTAAAACAGCAGAGGCACAACC | 727 |
| PS100s14 | TGTAAAACGACGGCCAGTTCAGTATCACAAAGTGAGTCTCAAG | 659 | CAGGAAACAGCTATGACCGAAAAGCAAGTTGTTAAAAAGAACA | 728 |
| PS1Q0s15 | TGTAAAACGACGGCCAGTTCAGTATCACAAAGTGAGTCTCAAG | 660 | CAGGAAACAGCTATGACCGAAAAGCAAGTTGTTAAAAAGAACA | 729 |
| PS100s16 | TGTAAAACGACGGCCAGTTCAGTATCACAAAGTGAGTCTCAAG | 661 | CAGGAAACAGCTATGACCGAAAAGCAAGTTGTTAAAAAGAACA | 730 |
| PS100s17 | TGTAAAACGACGGCCAGTTCAGTATCACAAAGTGAGTCTCAAG | 662 | CAGGAAACAGCTATGACCGAAAAGCAAGTTGTTAAAAAGAACA | 731 |
| PS100s18 | TGTAAAACGACGGCCAGTTCAGTATCACAAAGTGAGTCTCAAG | 663 | CAGGAAACAGCTATGACCGAAAAGCAAGTTGTTAAAAAGAACA | 732 |
| PS100s19 | TGTAAAACGACGGCCAGTCAGTACCCCTGACCTCTACCTT | 664 | CAGGAAACAGCTATGACCGCAGCTCTGTCACTCAGCTTT | 733 |
| PS100s20 | TGTAAAACGACGGCCAGTCAGGGTTTCTGMCATCCTCA | 665 | CAGGAAACAGCTATGACCTCTTGTTGGTTTTATTGACGGA | 734 |
| PS100s21 | TGTAAAACGACGGCCAGTTTCATCTTGAGAGAGGGGAGG | 666 | CAGGAAACAGCTATGACCTTGACGGAAGCTTTGAAATTG | 735 |
| PS100s22 | TGTAAAACGACGGCCAGTTCCCTGAGCTGTTATTGGAGA | 667 | CAGGAAACAGCTATGACCTTGTGGTTGTCATAACTGCACA | 736 |
| PS100s23 | TGTAAAACGACGGCCAGTCATCACACCCATCACMTMCAG | 668 | CAGGAAACAGCTATGACCTCTCCTCCCCTTCTTTGTCTT | 737 |
| PS100s24 | TGTAAAACGACGGCCAGTCCTGCTAGACAGGGTGAGATG | 669 | CAGGAAACAGCTATGACCCATTTCGTCATCATCAAAGCA | 738 |
| PS100s25 | TGTAAAACGACGGCCAGTGAAAGTTTAGTTAAAATTGTTTTCTGC | 670 | CAGGAAACAGCTATGACCTTGACATACATTGTGTTTCATCTATAAA | 739 |
| PS100s26 | TGTAAAACGACGGCCAGTCAAACCCATCATAGGTCATGG | 671 | CAGGAAACAGCTATGACCTTGACATACATTGTGTTTCATCTATAAA | 740 |
| PS100s27 | TGTAAAACGACGGCCAGTATTCCAACGGTGTTCAGTTTG | 672 | CAGGAAACAGCTATGACCGXAGCAAGGGGAGGAAGMC | 741 |
| PS100s28 | TGTAAAACGACGGCCAGTGGGCGATTCTTTTACAACTGA | 673 | CAGGAAACAGCTATGACCGCCCAAGAGATGATGCTTGTA | 742 |
| PS100s29 | TGTAAAACGACGGCCAGTGACAAAGGGAAAGTGATCATACAA | 674 | CAGGAAACAGCTATGACCTTGTCAAAGTTTGCPAAGTGA | 743 |
| PS100s30 | TGTAAAACGACGGCCAGTTAGATGCCAAGAATGCATGGT | 675 | CAGGAAACAGCTATGACCAAAATTAATGTTTAAAATGAAACACTCTC | 744 |
| PS100s31 | TGTAAAACGACGGCCAGTTAGATGCCPAGAATGCATGGT | 676 | CAGGAAACAGCTATGACCTGAAACACTCTCTTATCTACATAGGTTG | 745 |
| PS100s32 | TGTAAAACGACGGCCAGTTTGGACCAAGCTCATGAAAAT | 677 | CAGGAAACAGCTATGACCTAAGAACCATGCATTCTTGGC | 746 |
| PS100s33 | TGTAAAACGACGGCCAGTGCCCACTGGAAACTTAACACA | 678 | CAGGAPACAGCTATGACCGGTAGAGGTTAAAAAGGAGGAGC | 747 |
| PS100s34 | TGTAAAACGACGGCCAGTTTGACAATCACAGCTTGCAAA | 679 | CAGGAAACAGCTATGACCAPAGTGAGAGACATGGTTACTGTG | 748 |
| PS100s35 | TGTAAAACGACGGCCAGTTTGACAATCACAGCTTGCAAA | 680 | CAGGAAACAGCTATGACCAAAGTGAGAGACATGGTTACTGTG | 749 |
| PS100s36 | TGTAAAACGACGGCCAGTCCMGCTAGACTTCAGGCCTT | 681 | CAGGAMCAGCTATGACCTTTGAGGAGTTCCTGGTCCTT | 750 |
| PS101s1 | TGTAAAACGACGGCCAGTATTTCACACCACTAGCCATGC | 682 | CAGGAAACAGCTATGACCAGAAAGGAGGMGATGGTGGA | 751 |
| PS101s2 | TGTAAAACGACGGCCAGTACAGCTGCGGTAAGTCTGTGT | 683 | CAGGAAACAGCTATGACCCTCATCTTGTCTCCTTGCCAG | 752 |
| PS101s3 | TGTAAAACGACGGCCAGTGCAACGCTAAAGAATCGTCTG | 684 | CAGGAAACAGCTATGACCCCTCACAAACTGCCTCTTCAG | 753 |
| PS101s4 | TGTAAAACGACGGCCAGTTGGAATTGACAGTGCTGACTG | 685 | CAGGAAACAGGTATGACCCACTGCCTCTTACCTCCTGTG | 754 |
| PS101s5 | TGTAAAACGACGGCCAGTTGGAATTGACAGTGCTGACTG | 686 | CAGGAAACAGCTATGACCCACTGCCTCTTACCTCCTGTG | 755 |
| PS101s6 | TGTAAAACGACGGCCAGTTAAACCTTCTGCGATCAGGTG | 687 | CAGGAAACAGCTATGAGCGGGGGTTTTGAAAGTCTGATC | 756 |
| PS101s7 | TGTAAAACGACGGCCAGTGTTCGTTTTCATTTGCGTGAT | 688 | CAGGAAACAGCTATGACCAAATCCAAGATCCTGGTCCTG | 757 |
| PS101s8 | TGTAAAACGACGGCCAGTGTTCGTTTTCATTTGCGTGAT | 689 | CAGGAAACAGCTATGACCCTGCGGTGGATCTAGAGACAG | 758 |
| PS101s9 | TGTAAAACGACGGCCAGTTGAGGCTCAGGACAGTTAGGA | 690 | CAGGAAACAGCTATGACCCACTGCACAGTGATCACCATC | 759 |
| PS101s10 | TGTAAAACGACGGCCAGTATTTCACACCACTAGCCATGC | 691 | CAGGAAACAGCTATGACCAGAAAGGAGGAAGATGGTGGA | 760 |
| PS101s11 | TGTAAAACGACGGCCAGTATTTGCAAAGGACAGAGGACA | 692 | CAGGAAACAGCTATGACCATCTGCTTGCAAGMGACCCT | 761 |
| PS101s12 | TGTAAAACGACGGCCAGTCACAGCCTCTGCTAACAGGTT | 693 | CAGGMACAGCTATGACCATCTGCTTGCAAGAAGACGCT | 762 |
| PS101s13 | TGTAAAACGACGGCCAGTAAATTTGTTTCACCCCATACC | 694 | CAGGAXACAGCTATGACCTCACTGCATACCGTTTTTCCT | 763 |
| PS101s14 | TGTAAAACGACGGCCAGTTAATGGAGGTGAAGGCCTTTT | 695 | CAGGAAACAGCTATGAGCTGTGGTATCAGGAAGGATTGG | 764 |
| PS101s15 | TGTAAAACGACGGCCAGTAAGCCAGATTCCAATGTTCCT | 696 | CAGGAAACAGCTATGACCATTCTCACTGCCTGGCCTATT | 765 |
| PS101s16 | TGTAAAACGACGGCCAGTATCCACAATCTGCACACACAA | 697 | CAGGAAACAGCTATGACCCAGTTTTCTGAGGCCTCCTTT | 766 |
| PS101s17 | TGTAAAACGACGGCCAGTTTCGACGAAAGCTGTTCTCTC | 698 | CAGGAAACAGCTATGACCAAAGGAACCTGGGCATTTCTA | 767 |
| PS101s18 | TGTAAAACGACGGCCAGTTTCGACGAAAGCTGTTCTCTC | 699 | CAGGAAACAGCTATGAGCAAAGGAACGTGGGCATTTCTA | 768 |
| PS101s19 | TGTAAAACGACGGCCAGTTTCTGGTTCTTGTTGGTGACC | 700 | CAGGAAACAGCTATGACCGGCCTCTTGTACTTTGGGAAC | 769 |
| PS101s20 | TGTAAAACGACGGCCAGTGATGCACTCTCGAAGGAGTTG | 701 | CAGGAAACAGCTATGACCCGCGTGAGCACCTAATTAGTC | 770 |
| PS101s21 | TGTAAAACGACGGCCAGTCCAATCCTTCCTGATACCACA | 702 | CAGGAAACAGCTATGACCGGGAACCTTCATTCAGAGACC | 771 |
| PS101s22 | TGTAAAACGACGGCCAGTACTAGCCCTCAGTGCCTTCTC | 703 | CAGGAAACAGCTATGACCTCCAATCTTGAGGGGAAATCT | 772 |
| PS101s23 | TGTAAAACGACGGCCAGTCTTGGTGGAGAGTATCGCAA | 704 | CAGGAAACAGCTATGACCGGAGCACATCCTTCCATTGTA | 773 |
| PS101s24 | TGTAAAACGACGGCCAGTGGTCCCAACTCTCTCCATAGG | 705 | CAGGAAACAGCTATGACCTGGGTAGAGTTGGAGGAAGGT | 774 |
| PS101s25 | TGTAAAACGACGGCCAGTAGTTCTGCTGGGAGCTCTTCT | 706 | CAGGAAACAGCTATGACCAGCTGTCTTCCCCTTCTCTTG | 775 |
| PS101s26 | TGTAAAACGACGGCCAGTCTAATCGTCATGGGGGTTCTT | 707 | CAGGAAACAGCTATGACCCACCATCATCGTCAUCCTCT | 776 |
| PS101s27 | TGTAAAACGACGGCCAGTCTAATCGTCATGGGGGTTCTT | 708 | CAGGAAACAGCTATGACCCACCATCATCGTCATTCCTCT | 777 |
| PS101s28 | TGTAAAACGACGGCCAGTCTAATCGTCATGGGGGTTCTT | 709 | CAGGMACAGCTATGACCCACCATCATCGTCATTCCTCT | 778 |
| PS101s29 | TGTAAAACGACGGCCAGTGAAATTGGAAAGTGCCACAGA | 710 | CAGGAAACAGCTATGACCAGTGTGGTCCATGGAGATGAG | 779 |
| PS101s30 | TGTAAAACGACGGCCAGTGCCCAGGCATAGAGTTTCAAT | 711 | CAGGAAACAGCTATGACCAGCATGCACTTTCTTCCTCAA | 780 |
| PS101s31 | TGTAAAACGACGGCCAGTGTTGTGCCACTGGTGTTTCTT | 712 | CAGGMACAGCTATGACCGGACAAGATAGMCTTGGGCC | 781 |
| PS101s32 | TGTAAAACGACGGCCAGTACTTGTGCCCAGGACATAATG | 713 | CAGGAAACAGCTATGACCCTCCTTGTGGTTGGCATTCTA | 782 |
| PS101s33 | TGTAAAACGACGGCCAGTTCCCTCAATCTCCAGGATTCT | 714 | CAGGAAACAGCTATGACCATTTCTGGAGTGCCTTTGGTT | 783 |

What is claimed is:

1.) An isolated nucleic acid derived from a human gene encoding a protein selected from a member of the group consisting of the human OATP2 protein, and human cMOAT protein, wherein said nucleic acid comprises at least one polymorphic position.

2.) The isolated nucleic acid of claim 1 wherein said at least one polymorphic position for each said gene is a polymorphic position specified in Table IV, V, or complement thereof.

3.) The isolated nucleic acid of claim 2 wherein the sequence at said at least one polymorphic position is depicted in a nucleic acid sequence selected from the group consisting of SEQ ID NO: 5, 7, 9, 11, 13, 15, 17, 19, 21,23, 25, 27, 29, 31,33, 35,37, 39,41,43,45,47,49, 603, 51 to 326, or complement thereof.

4.) The isolated nucleic acid of claim 3 wherein said at least one polymorphic position resides in a member of the group consisting of: a non-coding position within the genomic sequence of said gene; a coding position within the genomic sequence of said gene; a coding position which results in a missense mutation of the translated product of said gene; a coding position that results in a silent mutation of the translated product of said gene; a non-coding position that resides within the untranslated region of said gene; and a non-coding position that resides within an intronic region of said gene.

5.) The isolated nucleic acid molecule according to claim 4, wherein said nucleic acid sequence is at least 15 nucleotides in length.

6.) The isolated nucleic acid molecule according to claim 4, wherein said nucleic acid sequence is at least 30 nucleotides in length.

7.) The isolated nucleic acid molecule according to claim 4, wherein said nucleic acid sequence is at least 40 nucleotides in length.

8.) A probe that hybridizes to a polymorphic position defined in claim 2.

9.) The probe of claim 8 wherein said probe is at least 15 nucleotides in length.

10.) The probe of claim 9 wherein a central position of the probe aligns with said polymorphic position.

11.) The probe of claim 9 wherein the 3′ end of the primer aligns with said polymorphic position.

12.) A method of analyzing at least one nucleic acid sample, comprising the steps of (1) obtaining a nucleic acid sample from one or more individuals; and (2) determining the nucleic acid sequence at one or more polymorphic positions in a gene encoding a protein selected from the group consisting of the human OATP2 protein, and human cMOAT protein, wherein the presence of a reference allele at said position(s) correlates to a phenotype.

13.) A method of identifying at least one nucleic acid sample that correlates to a phenotype, comprising the steps of (1) obtaining said nucleic acid sample from one or more individuals; and (2) determining the nucleic acid sequence at one or more polymorphic positions in a gene encoding a protein selected from the group consisting of the human OATP2 protein, and human cMOAT protein, wherein the presence of a alternate allele at said position(s) correlates to said phenotype.

14.) The method according to claim 12, wherein the phenotype is selected from the group consisting of: low hepatic statin uptake, decreased statin response; increased risk of developing drug interactions upon administration of at least one statin; increased susceptibility for developing a cardiovascular disorder; increased susceptibility for developing high cholesterol levels, metabolic dieases, inflammatory diseases, hypertension, and congestive heart failure; increased susceptibility for having low efficious response to pravastatin therapy; increased susceptibility for having decreased ability to transport compounds by organic anion transporters; increased susceptibility for having decreased ability to transport compounds by organic anion transporters in the liver; increased susceptibility for developing liver disease; increased susceptibility for developing a disease associated with low levels of low-density lipoprotein cholesterol; increased susceptibility to develop multidrug resistance; increased susceptibility to to have decreased response to HMG-CoA reductase inhibitor therapy; increased susceptibility for developing a disorder due to decreased hepatic or cellular uptake of taurocholate, estrone sulfate, estradiol 17-D-glucuronide, leukotriene C4, prostaglandin E2, or thyroid hormone.

15.) The method according to claim 13, wherein the phenotype is selected from the group consisting of: low hepatic statin uptake, decreased statin response; increased risk of developing drug interactions upon administration of at least one statin; increased susceptibility for developing a cardiovascular disorder; increased susceptibility for developing high cholesterol levels, metabolic dieases, inflammatory diseases, hypertension, and congestive heart failure; increased susceptibility for having low efficious response to pravastatin therapy; increased susceptibility for having decreased ability to transport compounds by organic anion transporters; increased susceptibility for having decreased ability to transport compounds by organic anion transporters in the liver; increased susceptibility for developing liver disease; increased susceptibility for developing a disease associated with low levels of low-density lipoprotein cholesterol; increased susceptibility to develop multidrug resistance; increased susceptibility to to have decreased response to HMG-CoA reductase inhibitor therapy; increased susceptibility for developing a disorder due to decreased hepatic or cellular uptake of taurocholate, estrone sulfate, estradiol 1 7-D-glucuronide, leukotriene C4, prostaglandin E2, or thyroid hormone.

16.) A kit for identifying an individual at risk of developing a phenotype, said kit comprising
i.) sequencing primers, and
ii.) sequencing reagents,
wherein said primers are primers that hybridize to at least one polymorphic position in a human gene selected from the group consisting of human OATP2 protein, and human cMOAT protein.

17.) The kit according to claim 16 wherein said polymorphic positions are selected from a group consisting of the polymorphic positions provided in Table IV, or V.

18.) The kit according to claim 17 wherein the sequence at said at least one polymorphic position is depicted in a nucleic acid sequence selected from the group consisting of SEQ ID NO: 5, 7, 9, 11, 13, 15, 17, 19, 21, 23, 25, 27, 29, 31, 33, 35, 37, 39, 41, 43, 45, 47, 49, 603, 51 to 326, or complement thereof.

19.) The kit according to claim 17 wherein the wherein the phenotype is selected from the group consisting of: low hepatic statin uptake, decreased statin response; increased risk of developing drug interactions upon administration of at least one statin; increased susceptibility for developing a cardiovascular disorder; increased susceptibility for developing high cholesterol levels, metabolic dieases, inflammatory diseases, hypertension, and congestive heart failure; increased susceptibility for having low efficious response to pravastatin therapy; increased susceptibility for having decreased ability to transport compounds by organic anion transporters; increased susceptibility for having decreased ability to transport compounds by organic anion transporters in the liver; increased susceptibility for developing liver disease; increased susceptibility for developing a disease associated with low levels of low-density lipoprotein cholesterol; increased susceptibility to develop multidrug resistance; increased susceptibility to to have decreased response to HMG-CoA reductase inhibitor therapy; increased susceptibility for developing a disorder due to decreased hepatic or cellular uptake of taurocholate, estrone sulfate, estradiol 17-D-glucuronide, leukotriene C4, prostaglandin E2, or thyroid hormone.
